(19) United States
(12) Patent Application Publication
(10) Pub. No.: US 2026/0047580 A1
(43) Pub. Date: Feb. 19, 2026

(54) HETEROCYCLIC COMPOUND AND HARMFUL ARTHROPOD PEST CONTROL COMPOSITION CONTAINING SAME

(71) Applicant: SUMITOMO CHEMICAL COMPANY, LIMITED, Chuo-ku (JP)

(72) Inventors: Takeshi TSURUDA, Takarazuka-shi (JP); Yoshihiko NOKURA, Takarazuka-shi (JP); Yasumasa SAITO, Takarazuka-shi (JP)

(73) Assignee: SUMITOMO CHEMICAL COMPANY, LIMITED, Chuo-ku (JP)

(21) Appl. No.: 19/359,867

(22) Filed: Oct. 16, 2025

Related U.S. Application Data
(63) Continuation of application No. 17/598,744, now Pat. No. 12,478,067.

(30) Foreign Application Priority Data
Mar. 29, 2019 (JP) 2019-066002
Dec. 26, 2019 (JP) 2019-235932

(51) Int. Cl. A01N 43/90; A01N 43/54; A01P 7/00; C07D 401/04; C07D 471/04; C07D 519/00

(52) U.S. Cl. CPC A01N 43/90; A01N 43/54; A01P 7/00; C07D 401/04; C07D 471/04; C07D 519/00

(57) ABSTRACT

A compound of formula (I) or an N-oxide thereof, a composition for controlling harmful arthropods including the compound, and a method for controlling harmful arthropods by applying the compound,

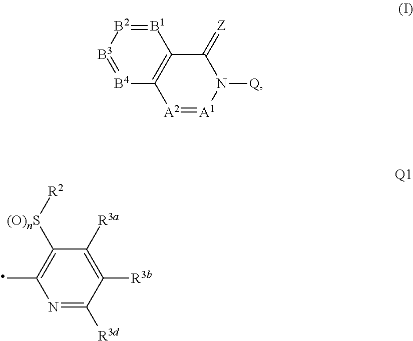

where Q represents a group represented by formula Q1 etc., Z represents an oxygen atom etc., A¹ represents CR^{4a} etc., A² represents a nitrogen atom etc., B¹ represents a nitrogen atom or CR^{6a} etc., B² represents CR¹ etc., B³ represents a nitrogen atom or CR^{6c} etc., B⁴ represents a nitrogen atom or CR^{6d} etc., R¹ represents a C1-C6 chain hydrocarbon group etc., R² represents a C1-C6 alkyl group etc., R^{3a}, R^{3b} and R^{3d} are identical to or different from each other and each represents a C1-C6 chain hydrocarbon group etc., R^{4a}, R^{6a}, R^{6c} and R^{6d} are identical to or different from each other and each represents a C1-C6 chain hydrocarbon group etc., and n is 0, 1 or 2.

### TECHNICAL FIELD

This application claims priority to and the benefit of Japanese Patent Application No. 2019-066002 filed Mar. 29, 2019, and Japanese Patent Application No. 2019-235932 filed Dec. 26, 2019, the entire contents of which are incorporated herein by reference.

The present invention is related to a certain class of heterocyclic compound and a composition for controlling harmful arthropods comprising the same.

### BACKGROUND ART

To date, in order to control harmful arthropods, some compounds have been studied. For example, a certain class of compound has been known to have an effect on controlling pests (see Patent Document 1).

### CITATION LIST

### Patent Document

Patent Document 1: WO 2016/129684

### SUMMARY OF THE INVENTION

### Problems to be Solved by Invention

An object of the present invention is to provide a compound having an excellent efficacy for controlling harmful arthropods.

### Means to Solve Problems

The present inventors have intensively studied to find compounds having an excellent efficacy for controlling harmful arthropods, and as a result, found that a compound represented by the below-mentioned formula (I) has an excellent efficacy for controlling harmful arthropods.

That is, the present invention includes the followings.

[1]A compound represented by formula (I):

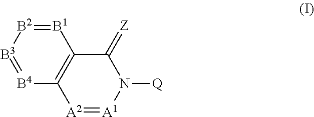

[wherein
Q represents a group represented by formula Q1, or a group represented by formula Q2,

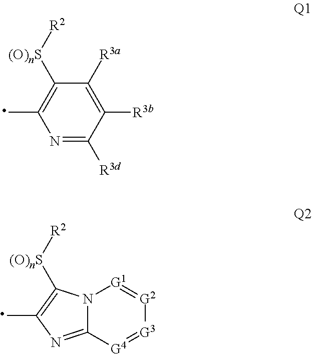

Z represents an oxygen atom or a sulfur atom,
a combination of A¹ and A² represents
   a combination in which A¹ represents CR^{4a}, and A² represents a nitrogen atom or CR^{4b}; or
   a combination in which A¹ represents a nitrogen atom, and A² represents a nitrogen atom or CR^{4b},
a combination of B¹, B², B³ and B⁴ represents,
   a combination in which B¹ represents a nitrogen atom or CR^{6a}, B² represents CR¹, B³ represents a nitrogen atom or CR^{6c}, and B⁴ represents a nitrogen atom or CR^{6d};
   a combination in which B¹ represents a nitrogen atom or CR^{6a}, B² represents a nitrogen atom or CR^{6b}, B³ represents CR¹, and B⁴ represents a nitrogen atom or CR^{6d};
   a combination in which B¹ represents a nitrogen atom or CR^{6a}, B² represents a nitrogen atom or CR^{6b}, B³ represents CR⁶C, and B⁴ represents CR¹;
   a combination in which B¹ represents a nitrogen atom or CR^{6a}, B² represents CR^{6b}, B³ represents a nitrogen atom, and B⁴ represents CR¹; or
   a combination in which B¹ represents CR^{6a}, B² represents a nitrogen atom, B³ represents a nitrogen atom, and B⁴ represents CR¹,
R¹ represents a C1-C6 chain hydrocarbon group having one or more substituents selected from the group consisting of cyano group and halogen atom, a C3-C4 cycloalkyl group optionally having one or more substituents selected from the group consisting of cyano group and halogen atom, S(O)ₘR⁸, OR⁸, halogen atom or OS(O)₂R⁸,
m represents 0, 1 or 2,
R⁸ represents a C1-C6 chain hydrocarbon group having one or more substituents selected from the group consisting of cyano group and halogen atom; or a C3-C4 cycloalkyl group optionally having one or more substituents selected from the group consisting of cyano group and halogen atom,
R^{4a}, R^{4b}, R^{6a}, R^{6b}, R^{6c} and R^{6d} are identical to or different from each other and each represents a C1-C6 chain hydrocarbon group optionally having one or more halogen atoms, a C3-C7 cycloalkyl group optionally having one or more halogen atoms, a C1-C6 alkoxy group optionally having one or more halogen atoms, NR⁹R¹⁰, C(O)R⁷, C(O)NR¹⁹R²⁰, NR⁹C(O)R¹⁸, NR⁹C(O)OR¹⁸, NR⁹C(O)NR¹⁹R²⁰, cyano group, halogen atom or a hydrogen atom,
R⁹ and R¹⁹ are identical to or different from each other and each represents a C1-C6 chain hydrocarbon group optionally having one or more halogen atoms, or a hydrogen atom,
R¹⁰ represents a C1-C6 chain hydrocarbon group optionally having one or more substituents selected from Group F, a C3-C7 cycloalkyl group optionally having one or more substituents selected from Group J, a C3-C7 cycloalkenyl group optionally having one or more substituents selected from Group J, a phenyl group optionally having one or more substituents selected from Group D, a six (6) membered aromatic heterocyclic group optionally having one or more substituents selected from Group D, a hydrogen atom, or S(O)₂R²¹,
R²¹ represents a C1-C6 chain hydrocarbon group optionally having one or more halogen atoms, a C3-C7 cycloalkyl group optionally having one or more halogen atoms, or a phenyl group optionally having one or more substituents selected from Group D,
R⁷, R¹⁸, and R²⁰ are identical to or different from each other and each represents a C1-C6 chain hydrocarbon group optionally having one or more substituents selected from Group F, a C3-C7 cycloalkyl group optionally having one or more substituents selected from Group J, or a hydrogen atom,
n represents 0, 1 or 2,
R² represents a C1-C6 chain hydrocarbon group optionally having one or more halogen atoms, a cyclopropyl group, or a cyclopropylmethyl group,
G¹ represents a nitrogen atom or CR^{3a},
G² represents a nitrogen atom or CR^{3b},
G³ represents a nitrogen atom or CR^{3c},
G⁴ represents a nitrogen atom or CR^{3d},
R^{3a}, R^{3b}, R^{3c} and R^{3d} are identical to or different from each other and each represents a C1-C6 chain hydrocarbon group optionally having one or more substituents selected from Group B, a C3-C7 cycloalkyl group optionally having one or more substituents selected from Group E, a phenyl group optionally having one or more substituents selected from Group H, a five (5) or six (6) membered aromatic heterocyclic group optionally having one or more substituents selected from Group H, OR¹², NR¹¹R¹², NR^{11a}R^{12a}, NR²⁴NR¹¹R¹², NR²⁴OR¹¹, NR¹¹C(O)R¹³, NR²⁴NR¹¹C(O)R¹³, NR¹¹C(O)OR¹⁴, NR²⁴NR¹¹C(O)OR¹⁴, NR¹¹C(O)NR³¹R³², NR²⁴NR¹¹C(O)NR³¹R³², N═CHNR³¹R³², N═S(O)ₚR¹⁵R¹⁶, C(O)R¹³, C(O)OR¹⁷, C(O)NR³¹R³², C(O)NR¹¹S(O)₂R²³, CR³⁰═NOR¹⁷, NR¹¹CR²⁴═NOR¹⁷, S(O)_{q}R²³, a cyano group, a nitro group, a hydrogen atom, or a halogen atom,
p represents 0 or 1,
q represents 0 or 1,
R^{3c} represents a C1-C6 chain hydrocarbon group optionally having one or more halogen atoms, a halogen atom OR³⁵, NR³⁶R³⁷, or a hydrogen atom,
R³⁵ represents a C1-C6 chain hydrocarbon group optionally having one or more halogen atoms,
R¹⁷ represents a C1-C6 chain hydrocarbon group optionally having one or more halogen atoms, a phenyl group optionally having one or more substituents selected from Group D, or a hydrogen atom,
R¹¹, R²⁴, R³⁶ and R³⁷ are identical to or different from each other and each represents a C1-C6 chain hydrocarbon group optionally having one or more halogen atoms, or a hydrogen atom,
R¹² represents a C1-C6 chain hydrocarbon group optionally having one or more substituents selected from Group F, a C3-C7 cycloalkyl group optionally having one or more substituents selected from Group J, a C3-C7 cycloalkenyl group optionally having one or more substituents selected from Group J, a phenyl group optionally having one or more substituents selected from Group D, a six (6) membered aromatic heterocyclic group optionally having one or more substituents selected from Group D, a hydrogen atom, or S(O)₂R²³,
R²³ represents a C1-C6 chain hydrocarbon group optionally having one or more halogen atoms, or a phenyl group optionally having one or more substituents selected from Group D,
R^{11a} and R^{12a} combined together with a nitrogen atom to which they are attached represent a three (3) to seven (7) membered nonaromatic heterocyclic group optionally having one or more substituents selected from Group E,
R¹³ represents a C1-C6 chain hydrocarbon group optionally having one or more halogen atoms, a C3-C7 cycloalkyl group optionally having one or more halogen atoms, a (C3-C6 cycloalkyl)C1-C3 alkyl group optionally having one or more halogen atoms, a phenyl group optionally having one or more substituents selected from Group D, a five (5) or six (6) membered aromatic heterocyclic group optionally having one or more substituents selected from Group D, or a hydrogen atom,
R¹⁴ represents a C1-C6 chain hydrocarbon group optionally having one or more halogen atoms, a C3-C7 cycloalkyl group optionally having one or more halogen atoms, a (C3-C6 cycloalkyl)C1-C3 alkyl group optionally having one or more halogen atoms, or a phenyl C1-C3 alkyl group {the phenyl moiety in the phenyl C1-C3 alkyl group may have optionally one or more substituents selected from Group D},
R¹⁵ and R¹⁶ are identical to or different from each other and each represents a C1-C6 alkyl group optionally having one or more halogen atoms,
R³¹ represents a C1-C6 alkyl group optionally having one or more halogen atoms, or a hydrogen atom,
R³² represents a C1-C6 chain hydrocarbon group optionally having one or more substituents selected from Group F, a C3-C7 cycloalkyl group optionally having one or more substituents selected from Group J, or a hydrogen atom,
when Q represents a group represented by formula Q1, R^{3b} and R^{3d} combined together with two carbon atoms to which they are attached represent a benzene ring, a pyrrole ring, a furan ring, a thiophene ring, a pyrazole ring, an imidazole ring, an oxazole ring, an isoxazole ring, a thiazole ring, an isothiazole ring, an oxadiazole ring, a thiadiazole ring, a pyridine ring, a pyridazine ring, a pyrimidine ring, a pyrazine ring {the benzene ring, the pyrrole ring, the furan ring, the thiophene ring, the pyrazole ring, the imidazole ring, the oxazole ring, the isoxazole ring, the thiazole ring, the isothiazole ring, the pyridine ring, the pyridazine ring, the pyrimidine ring, and the pyrazine ring each may have optionally one or more substituents selected from Group H}, or a triazole ring optionally having one or more substituents selected from Group I,
Group B: a group consisting of a C1-C6 alkoxy group optionally having one or more halogen atoms, a C3-C6 alkenyloxy group optionally having one or more halogen atoms, a C3-C6 alkynyloxy group optionally having one or more halogen atoms, a C1-C6 alkylsulfanyl group optionally having one or more halogen atoms, a C1-C6 alkylsulfinyl group optionally having one or more halogen atoms, a C1-C6 alkylsulfonyl group optionally having one or more halogen atoms, a C3-C6 cycloalkyl group optionally having one or more halogen atoms, a C1-C6 alkylamino group optionally having one or more halogen atoms, a di(C1-C4 alkyl)amino group optionally having one or more halogen atoms, a C2-C6 alkylcarbonyl group optionally having one or more halogen atoms, a C2-C6 alkoxycarbonyl group optionally having one or more halogen atoms, a C2-C6 alkoxycarbonyloxy group optionally having one or more halogen atoms, an aminocarbonyl group, a C1-C6 alkylaminocarbonyl group optionally having one or more halogen atoms, a di(C1-C4 alkyl)aminocarbonyl group optionally having one or more halogen atoms, a C2-C6 alkoxycarbonylamino group optionally having one or more halogen atoms, a (C2-C6 alkoxycarbonyl) (C1-C6 alkyl) amino group optionally having one or more halogen atoms, a cyano group, an amino group, a nitro group, a hydroxy group, and a halogen atom,
Group D: a group consisting of a C1-C6 chain hydrocarbon group optionally having one or more halogen atoms, a C1-C6 alkoxy group optionally having one or more halogen atoms, a C3-C6 alkenyloxy group optionally having one or more halogen atoms, a C3-C6 alkynyloxy group optionally having one or more halogen atoms, a C1-C6 alkylsulfanyl group optionally having one or more halogen atoms, a C1-C6 alkylsulfinyl group optionally having one or more halogen atoms, a C1-C6 alkylsulfonyl group optionally having one or more halogen atoms, a C3-C6 cycloalkyl group optionally having one or more halogen atoms, a C1-C6 alkylamino group optionally having one or more halogen atoms, a di(C1-C4 alkyl)amino group optionally having one or more halogen atoms, a C2-C6 alkylcarbonyl group optionally having one or more halogen atoms, a C2-C6 alkoxycarbonyl group optionally having one or more halogen atoms, a C2-C6 alkoxycarbonyloxy group optionally having one or more halogen atoms, an aminocarbonyl group, a C1-C6 alkylaminocarbonyl group optionally having one or more halogen atoms, a di(C1-C4 alkyl)aminocarbonyl group optionally having one or more halogen atoms, a C2-C6 alkoxycarbonylamino group optionally having one or more halogen atoms, a (C2-C6 alkoxycarbonyl) (C1-C6 alkyl) amino group optionally having one or more halogen atoms, a cyano group, an amino group, a nitro group, a hydroxy group, and a halogen atom,
Group E: a group consisting of a C1-C6 chain hydrocarbon group optionally having one or more halogen atoms, a C1-C6 alkoxy group optionally having one or more halogen atoms, a C3-C6 alkenyloxy group optionally having one or more halogen atoms, a C3-C6 alkynyloxy group optionally having one or more halogen atoms, a C1-C6 alkylamino group optionally having one or more halogen atoms, a di(C1-C4 alkyl)amino group optionally having one or more halogen atoms, a C2-C6 alkylcarbonyl group optionally having one or more halogen atoms, a C2-C6 alkoxycarbonyl group optionally having one or more halogen atoms, a C2-C6 alkoxycarbonyloxy group optionally having one or more halogen atoms, an aminocarbonyl group, a C1-C6 alkylaminocarbonyl group optionally having one or more halogen atoms, a di(C1-C4 alkyl)aminocarbonyl group optionally having one or more halogen atoms, a C2-C6 alkoxycarbonylamino group optionally having one or more halogen atoms, a (C2-C6 alkoxycarbonyl) (C1-C6 alkyl) amino group optionally having one or more halogen atoms, a cyano group, an amino group, a nitro group, a hydroxy group, an oxo group, and a halogen atom,
Group F: a group consisting of a C3-C7 cycloalkyl group optionally having one or more halogen atoms, a phenyl group optionally having one or more substituents selected from Group D, a five (5) or six (6) membered aromatic heterocyclic group optionally having one or more substituents selected from Group D, a C1-C6 alkoxy group optionally having one or more halogen atoms, a C1-C6 alkylamino group optionally having one or more halogen atoms, a di(C1-C4 alkyl)amino group optionally having one or more halogen atoms, a cyano group, an amino group, a nitro group, a hydroxy group, and a halogen atom,
Group H: a group consisting of a C1-C6 chain hydrocarbon group optionally having one or more halogen atoms, a C3-C6 cycloalkyl group optionally having one or more halogen atoms, a phenyl group optionally having one or more substituents selected from Group D, a five (5) or six (6) membered aromatic heterocyclic group optionally having one or more substituents selected from Group D, a C1-C6 alkoxy group optionally having one or more halogen atoms, a C1-C6 alkylamino group optionally having one or more halogen atoms, a di(C1-C4 alkyl)amino group optionally having one or more halogen atoms, a C2-C6 alkylcarbonyl group optionally having one or more halogen atoms, a C2-C6 alkoxycarbonyl group optionally having one or more halogen atoms, a C2-C6 alkoxycarbonyloxy group optionally having one or more halogen atoms, an aminocarbonyl group, a (C1-C6 alkyl)aminocarbonyl group optionally having one or more halogen atoms, a di(C1-C4 alkyl)aminocarbonyl group optionally having one or more halogen atoms, a C2-C6 alkoxycarbonylamino group optionally having one or more halogen atoms, a (C2-C6 alkoxycarbonyl) (C1-C6 alkyl) amino group optionally having one or more halogen atoms, a cyano group, an amino group, a nitro group, a hydroxy group, and a halogen atom,
Group I: a group consisting of a C2-C6 chain hydrocarbon group optionally having one or more halogen atoms, a C3-C6 cycloalkyl group optionally having one or more halogen atoms, a phenyl group optionally having one or more substituents selected from Group D, a five (5) or six (6) membered aromatic heterocyclic group optionally having one or more substituents selected from Group D, a C2-C6 alkylcarbonyl group optionally having one or more halogen atoms, a C2-C6 alkoxycarbonyl group optionally having one or more halogen atoms, an aminocarbonyl group, a (C1-C6 alkyl)aminocarbonyl group optionally having one or more halogen atoms, and a di(C1-C4 alkyl)aminocarbonyl group optionally having one or more halogen atoms,
Group J: a group consisting of a C1-C6 alkyl group optionally having one or more halogen atoms, a C1-C6 alkoxy group optionally having one or more halogen atoms, a C2-C6 alkoxycarbonyl group optionally having one or more halogen atoms, an amino group, a cyano group, and a halogen atom] (hereinafter, which is referred to as “present compound P” or “compound P of the present invention”) or N-oxide thereof (hereinafter, a compound represented by formula (I) or N-oxide thereof is collectively referred to as “present compound X” or “compound X of the present invention”).
   [2] The compound according to [1] wherein
a combination of A¹ and A² represents
   a combination in which A¹ represents CR^{4a}, and A² represents a nitrogen atom or CR^{4b}; or
   a combination in which A¹ represents a nitrogen atom, and A² represents CR^{4b},
R¹ represents a C1-C6 chain hydrocarbon group having one or more substituents selected from the group consisting of cyano group and halogen atom, a C3-C4 cycloalkyl group optionally having one or more substituents selected from the group consisting of cyano group and halogen atom, S(O)ₘR⁸, OR⁸, or OS(O)₂R⁸
   (hereinafter, which is referred to as “present compound N” or “compound N of the present invention”) or N-oxide thereof (hereinafter, a compound represented by formula (I) or N-oxide thereof is collectively referred to as “present compound” or “compound of the present invention”)
   [3] The compound according to [1] or [2] or N-oxide thereof, wherein
R^{3a} represents a hydrogen atom, R^{3b}, R^{3c} and R^{3d} are identical to or different from each other and each represents a C1-C6 alkyl group, a C2-C6 alkenyl group, a C3-C7 cycloalkyl group {the C1-C6 alkyl group, the C2-C6 alkenyl group, and the C3-C7 cycloalkyl group each may have optionally one or more substituents selected from the group consisting of halogen atom and cyano group}, a phenyl group, a triazolyl group, a pyridyl group, a pyrimidinyl group {the phenyl group, the triazolyl group, the pyridyl group, and the pyrimidinyl group each may have optionally one or more substituents selected from Group J}, OR¹², CR³═COR¹⁷, a hydrogen atom, or a halogen atom, and when Q represents a group represented by formula Q1, and R^{3b} and R^{3d} combined together with two carbon atoms to which they are attached may form a benzene ring {the benzene ring may have optionally one or more substituents selected from a group consisting of C1-C6 alkyl group optionally having one or more halogen atoms, and a halogen atom}.
   [4] The compound according to [1] or [2] or N-oxide thereof, wherein
R^{3a} and R^{3d} represent a hydrogen atom, R^{3b} and R^{3c} are identical to or different from each other and each represents a C1-C6 alkyl group optionally having one or more halogen atoms, a cyclopropyl group, a hydrogen atom, or a halogen atom, and when Q represents a group represented by formula Q1, R^{3b} and R^{3d} combined together with two carbon atoms to which they are attached may form a benzene ring {the benzene ring may have optionally one or more substituents selected from a group consisting of C1-C6 alkyl group optionally having one or more halogen atoms, and halogen atom}.
   [5] The compound according to any one of [1] to [4] or N-oxide thereof, wherein Q represents a group represented by formula Q1.
   [6] The compound according to [1] or [2] or N-oxide thereof, wherein Q represents a group represented by formula Q1, and
R^{3a} and R^{3d} represent a hydrogen atom.
   [7] The compound according to any one of [1] to [4] or N-oxide thereof, wherein Q represents a group represented by formula Q2.
   [8] The compound according to any one of [1] to [7] or N-oxide thereof, wherein B¹ represents CH, a combination of B², B³ and B⁴ represents a combination in which B² represents CR¹, B³ represents CR⁶C, and B⁴ represents CR^{6d}; a combination in which B² represents CR^{6b}, B³ represents CR¹, and B⁴ represents CR^{6d}; or a combination in which B² represents CR^{6b}, B³ represents CR⁶C, and B⁴ represents CR¹.
   [9] The compound according to any one of [1] to [4], [7] or [8] or N-oxide thereof, wherein G¹ represents a nitrogen atom or CH, G² represents CR^{3b}, G³ represents CR^{3c}, and G⁴ represents a nitrogen atom or CH.
   [10] The compound according to any one of [1] to [4], [6] or [7] or N-oxide thereof, wherein G¹ represents CH, G² represents CR^{3b}, G³ represents CR^{3c}, and G⁴ represents CH.
   [11] The compound according to any one of [1] to [10] or N-oxide thereof, wherein R¹ represents a C1-C6 alkyl group having one or more substituents selected from a group consisting of halogen atom and cyano group; a cyclopropyl group optionally having one or more substituents selected from the group consisting of cyano group and halogen atom; S(O)ₘR⁸; a halogen atom; or OR⁸.
   [12] The compound according to any one of [1] to [10] or N-oxide thereof, wherein R¹ represents a C1-C6 alkyl group having one or more substituents selected from a group consisting of halogen atom and cyano group; a cyclopropyl group optionally having one or more substituents selected from the group consisting of cyano group and halogen atom; S(O)ₘR⁸; or OR⁸.
   [13] The compound according to any one of [1] to [12] or N-oxide thereof, wherein R² represents an ethyl group.
   [14] The compound according to any one of [1] to [13] or N-oxide thereof, wherein Z represents an oxygen atom.
   [15]A composition for controlling harmful arthropod which comprises the compound according to any one of [1] to [14] or N-oxide thereof.
   [16]A composition comprising one or more ingredients selected from the group consisting of the following Group (a), Group (b), Group (c), and Group (d), and the compound according to any one of [1] to [14] or N-oxide thereof (hereinafter, which is referred to as “Present Composition” or “composition of the present invention”):
Group (a): a group consisting of insecticidal ingredients, miticidal ingredients, and nematicidal ingredients;
Group (b): fungicidal ingredients,
Group (c): plant growth modulating ingredients; and
Group (d): repellent ingredients.
   [17]A method for controlling harmful arthropod which comprises applying an effective amount of the compound according to any one of [1] to [14] or N-oxide thereof, or an effective amount of the composition according to [16] to a harmful arthropod or a habitat where a harmful arthropod lives.
   [18]A seed or vegetative reproductive organ carrying an effective amount of the compound according to any one of [1] to [14] or N-oxide thereof, or an effective amount of the composition according to [16].
   [19]A compound represented by formula (II):

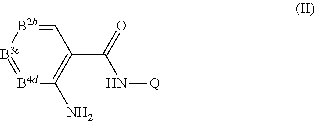

[wherein,
a combination of B^{2b}, B^{3c} and B^{4d} represents
   a combination in which B^{2b} represents CR¹, B^{3c} represents a nitrogen atom or CR^{6cc}, and B^{4d} represents a nitrogen atom or CR^{6dd};
   a combination in which B^{2b} represents a nitrogen atom or CR^{6bb}, B^{3c} represents CR¹, and B^{4d} represents a nitrogen atom or CR^{6dd}; or
   a combination in which B^{2b} represents a nitrogen atom or CR^{6bb}, B^{3c} represents a nitrogen atom or CR^{6cc}, and B^{4d} represents CR¹.

R^{6bb}, R^{6cc} and R^{6dd} are identical to or different from each other and each represents a C1-C6 chain hydrocarbon group optionally having one or more halogen atoms, a halogen atom or a hydrogen atom, and
the other symbols are the same as described in [1]] (hereinafter, which is referred to as “Intermediate compound C”), or salts thereof.
   [20] The compound according to [19] wherein
R¹ represents a C1-C6 chain hydrocarbon group having one or more substituents selected from the group consisting of cyano group and halogen atom, a C3-C4 cycloalkyl group optionally having one or more substituents selected from the group consisting of cyano group and halogen atom, S(O)ₘR⁸, OR⁸, or OS(O)₂R⁸
   (hereinafter, which is referred to as “Intermediate compound A”) or salts thereof.
   [21]A compound represented by formula (III):

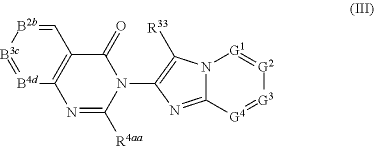

[wherein
R³³ represents a hydrogen atom or halogen atom,
a combination of B^{2b}, B^{3c} and B^{4d} represents
   a combination in which B^{2b} represents CR¹, B^{3c} represents a nitrogen atom or CR^{6cc}, and B^{4d} represents a nitrogen atom or CR^{6dd};
   a combination in which B^{2b} represents a nitrogen atom or CR^{6bb}, B^{3c} represents CR¹, and B^{4d} represents a nitrogen atom or CR^{6dd}; or
   a combination in which B^{2b} represents a nitrogen atom or CR^{6bb}, B^{3c} represents a nitrogen atom or CR^{6cc}, and B^{4d} represents CR¹;
R^{4aa}, R^{6bb}, R^{6cc} and R^{6dd} are identical to or different from each other and each represents a C1-C6 chain hydrocarbon group optionally having one or more halogen atoms, halogen atom or a hydrogen atom, and
the other symbols are the same as defined in [1].], (hereinafter, which is referred to as “Intermediate compound D”).
   [22] The compound according to [21], wherein R¹ represents a C1-C6 chain hydrocarbon group having one or more substituents selected from the group consisting of cyano group and halogen atom, a C3-C4 cycloalkyl group optionally having one or more substituents selected from the group consisting of cyano group and halogen atom, S(O)ₘR⁸, OR⁸, or OS(O)₂R⁸ (hereinafter, which is referred to as “Intermediate compound B”).

### Effect of Invention

The present invention can control harmful arthropod.

### MODE FOR CARRYING OUT THE INVENTION

The substituent(s) as described herein is/are explained.

The term “halogen atom” represents fluorine atom, chlorine atom, bromine atom, or iodine atom.

When the substituents have two or more halogen atoms, these halogen atoms may be identical to or different from each other.

The expression of “CX-CY” as used herein represents that the number of carbon atom is from X to Y. For example, the expression of “C1-C6” represents that the number of carbon atom is from 1 to 6.

The term of “chain hydrocarbon group” represents an alkyl group, an alkenyl group, or an alkynyl group.

Example of “alkyl group” include methyl group, ethyl group, propyl group, isopropyl group, 1,1-dimethylpropyl group, 1,2-dimethylpropyl group, 1-ethylpropyl group, butyl group, sec-butyl group, tert-butyl group, pentyl group, and hexyl group.

Example of “alkenyl group” include vinyl group, 1-propenyl group, 2-propenyl group, 1-methyl-1-propenyl group, 1-methyl-2-propenyl group, 1,2-dimethyl-1-propenyl group, 1-ethyl-1-propenyl group, 3-butenyl group, 4-pentenyl group, and 5-hexenyl group.

Example of “alkynyl group” includes ethynyl group, 1-propynyl group, 2-propynyl group, 1-methyl-2-propynyl group, 1,1-dimethyl-2-propynyl group, 1-ethyl-2-propynyl group, 2-butynyl group, 4-pentynyl group, and 5-hexynyl group.

Examples of “alkoxy group” includes methoxy group, ethoxy group, propoxy group, isopropoxy group, butoxy group, tert-butoxy group, pentoxy group, and hexyloxy group.

Examples of “alkenyloxy group” includes 2-propenyloxy group, 2-butenyloxy group, and 5-hexenyloxy group.

Examples of “alkynyloxy group” includes 2-propynyloxy group, 2-butynyloxy group, and 5-hexynyloxy group.

Examples of “fluoroalkyl group” include trifluoromethyl group, 2,2,2-trifluoroethyl group, pentafluoroethyl group, 2,2,3,3,3-pentafluoropropyl group, 1,1,1-trifluoropropan-2-yl group, and heptafluoropropyl group.

Examples of “fluoroalkoxy group” include fluoromethoxy group, difluoromethoxy group, trifluoromethoxy group, 2,2,2-trifluoroethoxy group, perfluoroethoxy group, and perfluoropropoxy group.

Examples of “cycloalkyl group” include cyclopropyl group, cyclobutyl group, cyclopentyl group, cyclohexyl group, and cycloheptyl group.

Examples of “cycloalkenyl group” include cyclopropenyl group, cyclobutenyl group, cyclopentenyl group, cyclohexenyl group, and cycloheptenyl group.

Examples of “three (3) to seven (7) membered nonaromatic heterocyclic group” include aziridine ring, azetidine ring, pyrrolidine ring, imidazoline ring, imidazolidine ring, piperidine ring, tetrahydropyrimidine ring, hexahydropyrimidine ring, piperazine ring, azepane ring, oxazolidine ring, isoxazolidine ring, 1,3-oxazinane ring, morpholine ring, 1,4-oxazepane ring, thiazolidine ring, isothiazolidine ring, 1,3-thiazinane ring, thiomorpholine ring, and 1,4-thiazepane ring.

Examples of the three (3) to seven (7) membered nonaromatic heterocyclic group optionally having one or more substituents selected from Group E include the followings:

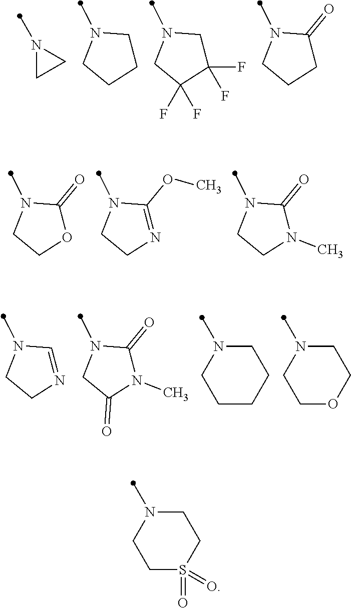

The term of “five (5) or six (6) membered aromatic heterocyclic group” represents five (5) membered aromatic heterocyclic group or six (6) membered aromatic heterocyclic group, and examples of the five (5) membered aromatic heterocyclic group include pyrrolyl group, furyl group, thienyl group, pyrazolyl group, imidazolyl group, triazolyl group, tetrazolyl group, oxazolyl group, isoxazolyl group, thiazolyl group, isothiazolyl group, oxadiazolyl group, and thiadiazolyl group. Examples of six (6) membered aromatic heterocyclic group include pyridyl group, pyridazinyl group, pyrimidinyl group, pyrazinyl group, triazinyl group, and tetrazinyl group.

Examples of “(C3-C6 cycloalkyl)C1-C3 alkyl group optionally having one or more halogen atoms” include cyclopropylmethyl group, (2-fluorocyclopropyl)methyl group, cyclopropyl(fluoro)methyl group, and (2-fluorocyclopropyl) (fluoro)methyl group.

Examples of “phenylC1-C3 alkyl group {the phenyl moiety in the phenyl C1-C3 alkyl group may have optionally one or more substituents selected from Group D}” include benzyl group, 2-fluorobenzyl group, 4-chlorobenzyl group, 4-(trifluromethyl)benzyl group, and 2-[4-(trifluoromethyl)phenyl]ethyl group.

The terms of “alkylsulfanyl group”, “alkylsulfinyl group” and “alkylsulfonyl group” represent an alkyl group containing a S(O)_{z} moiety, respectively.

For example, examples of the “alkylsulfanyl” when z is 0 include methylsulfanyl group, ethylsulfanyl group, propylsulfanyl group, and isopropylsulfanyl group.

For example, examples of the “alkylsulfinyl” when z is 1 include methylsulfinyl group, ethylsulfinyl group, propylsulfinyl group, and isopropylsulfinyl group.

For example, examples of the “alkylsulfonyl” when z is 2 include methylsulfonyl group, ethylsulfonyl group, propylsulfonyl group, and isopropylsulfonyl group.

Examples of “N-oxide of the compound represented by formula (I)” include compounds represented by the following formulae.

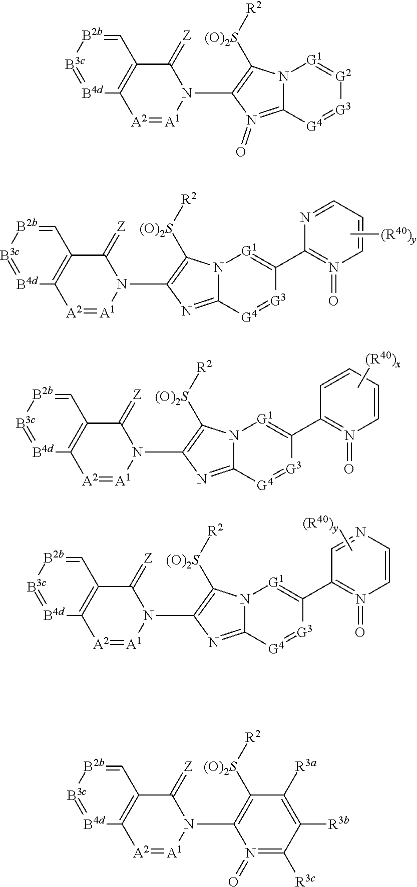

[wherein R⁴⁰ represents any substituents selected from Group H, x is 0, 1, 2, 3 or 4, y is 0, 1, 2 or 3, and the other symbols are the same as defined.]

The present compound X, the intermediate compound C, and the intermediate compound D may be existed as one or more stereoisomers. Examples of the stereoisomer include enantiomer, diastereoisomer, and geometric isomer. Each stereoisomer, and stereoisomer mixture(s) in an arbitrary ratio thereof are included in the present compound X, the intermediate compound C, and the intermediate compound D.

The present compound X, and the intermediate compound C may form acid addition salts. Examples of the acid to form the acid addition salt include inorganic acids such as hydrogen chloride, phosphoric acid, sulfuric acid; and organic acids such as acetic acid, trifluoroacetic acid, benzoic acid, p-toluenesulfonic acid. The acid addition salt may be obtained by mixing the present compound X or the intermediate compound C with an acid.

Examples of the Embodiment of the present compound N include the followings.

[Embodiment 1] The present compound N wherein R^{3a} represents a hydrogen atom, R^{3b}, R^{3c} and R^{3d} are identical to or different from each other and each represents a C1-C6 alkyl group, a C2-C6 alkenyl group, a C3-C7 cycloalkyl group {the C1-C6 alkyl group, the C2-C6 alkenyl group, and the C3-C7 cycloalkyl group each may have optionally one or more substituents selected from the group consisting of halogen atom and cyano group}, a phenyl group, a triazolyl group, a pyridyl group, a pyrimidinyl group {the phenyl group, the triazolyl group, the pyridyl group, and the pyrimidinyl group each may have optionally one or more substituents selected from Group J}, OR¹², CR^{3c}═NOR¹⁷, a hydrogen atom, or halogen atom, and when Q represents a group represented by formula Q1, and R^{3b} and R^{3d} combined together with two carbon atoms to which they are attached represent a benzene ring {the benzene ring may have optionally a C1-C6 alkyl group optionally having one or more halogen atoms and halogen atom}.
[Embodiment 2] The present compound N wherein R^{3a} and R^{3d} represent a hydrogen atom, R^{3b} and R^{3c} are identical to or different from each other and each represents a C1-C6 alkyl group optionally having one or more halogen atoms, a cyclopropyl group, a hydrogen atom, or a halogen atom, and when Q represents a group represented by formula Q1, R^{3b} and R^{3d} combined together with two carbon atoms to which they are attached represent a benzene ring {the benzene ring may have optionally one or more substituents selected from a group consisting of C1-C6 alkyl group optionally having halogen atoms, and halogen atom.
[Embodiment 3] The present compound N wherein Q represents a group represented by formula Q2.
[Embodiment 4] The compound according to the Embodiment 1 wherein Q represents a group represented by formula Q2.
[Embodiment 5] The compound according to the Embodiment 2 wherein Q represents a group represented by formula Q2.
[Embodiment 6] The present compound N wherein Q represents a group represented by formula Q1.
[Embodiment 7] The compound according to the Embodiment 1 wherein Q represents a group represented by formula Q1.
[Embodiment 8] The compound according to the Embodiment 2 wherein Q represents a group represented by formula Q1.
[Embodiment 9] The present compound N wherein Q represents a group represented by formula Q1, R^{3a} represents a hydrogen atom, R^{3b} and R^{3d} are identical to or different from each other and each represents a C1-C6 alkyl group, a C2-C6 alkenyl group, a C3-C7 cycloalkyl group {the C1-C6 alkyl group, the C2-C6 alkenyl group, and the C3-C7 cycloalkyl group each may have optionally one or more substituents selected from the group consisting of halogen atom and cyano group}, a phenyl group, a triazolyl group, a pyridyl group, a pyrimidinyl group {the phenyl group, the triazolyl group, the pyridyl group, and the pyrimidinyl group each may have optionally one or more substituents selected from Group J}, OR¹², CR³⁰═NOR¹⁷, a hydrogen atom, or a halogen atom.
[Embodiment 10] The present compound N wherein Q represents a group represented by formula Q1, R^{3a} and R^{3d} represent a hydrogen atom, R^{3b} represents a C1-C6 alkyl group optionally having one or more halogen atoms, a cyclopropyl group, a hydrogen atom, or a halogen atom.
[Embodiment 11] The present compound N wherein Q represents a group represented by formula Q1, R^{3a} represents a hydrogen atom, R^{3b} and R^{3d} combined together with two carbon atoms to which they are attached may form a benzene ring, a pyrrole ring, a furan ring, a thiophene ring, a pyrazole ring, an imidazole ring, an oxazole ring, an isoxazole ring, a thiazole ring, an isothiazole ring, an oxadiazole ring, a thiadiazole ring, a pyridine ring, a pyridazine ring, a pyrimidine ring, a pyrazine ring {the benzene ring, the pyrrole ring, the furan ring, the thiophene ring, the pyrazole ring, the imidazole ring, the oxazole ring, the isoxazole ring, the thiazole ring, the isothiazole ring, the pyridine ring, the pyridazine ring, the pyrimidine ring, and the pyrazine ring each may have optionally one or more substituents selected from Group H}, or a triazole ring optionally having one or more substituents selected from Group I.
[Embodiment 12] The present compound N wherein Q represents a group represented by formula Q1, R^{3a} represents a hydrogen atom, R^{3b} and R^{3d} combined together with two carbon atoms to which they are attached form a benzene ring {the benzene ring may have optionally one or more substituents selected from a group consisting of C1-C6 alkyl group optionally having one or more halogen atoms, and halogen atom}.
[Embodiment 13] The present compound N wherein G² represents CR^{3b}, G³ represents CR^{3c}, and a combination of G¹ and G⁴ represents a combination in which G¹ represents CR^{3a}, and G⁴ represents a nitrogen atom or CR^{3d}; or a combination in which G¹ represents a nitrogen atom, and G⁴ represents CR^{3d}.
[Embodiment 14] The present compound N wherein G¹ represents CH, G² represents CR^{3b}, G³ represents GR^{3c}, and G⁴ represents CH.
[Embodiment 15] The present compound N wherein G¹ represents a nitrogen atom, G² represents CR^{3b}, G³ represents GR^{3c}, and G⁴ represents CH.
[Embodiment 16] The present compound N wherein G¹ represents CH, G² represents CR^{3b}, G³ represents CR^{3c}, and G⁴ represents a nitrogen atom.
[Embodiment 17] The compound according to the Embodiment 1 wherein G² represents CR^{3b}, G³ represents CR^{3c}, and a combination of G¹ and G⁴ represents a combination in which G¹ represents CH, and G⁴ represents a nitrogen atom or CR^{3d}; or a combination in which G¹ represents a nitrogen atom, and G⁴ represents CR^{3d}.
[Embodiment 18] The compound according to the Embodiment 2 wherein G² represents CR^{3b}, G³ represents CR^{3c}, and a combination of G¹ and G⁴ represents a combination in which G¹ represents CH, and G⁴ represents a nitrogen atom or CH; or a combination in which G¹ represents a nitrogen atom, and G⁴ represents CH.
[Embodiment 19] The compound according to the Embodiment 3 wherein G² represents CR^{3b}, G³ represents CR^{3c}, and a combination of G¹ and G⁴ represents a combination in which G¹ represents CR^{3a}, and G⁴ represents a nitrogen atom or CR^{3d}; or a combination in which G¹ represents a nitrogen atom, and G⁴ represents CR^{3d}.
[Embodiment 20] The compound according to the Embodiment 4 wherein G² represents CR^{3b}, G³ represents CR^{3c}, and a combination of G¹ and G⁴ represents a combination in which G¹ represents CH, and G⁴ represents a nitrogen atom or CR^{3d}; or a combination in which G¹ represents a nitrogen atom, and G⁴ represents CR^{3d}.
[Embodiment 21] The compound according to the Embodiment 5 wherein G² represents CR^{3b}, G³ represents CR^{3c}, and a combination of G¹ and G⁴ represents a combination in which G¹ represents CH, and G⁴ represents a nitrogen atom or CH; or a combination in which G¹ represents a nitrogen atom, and G⁴ represents CR^{3d}.
[Embodiment 22] The compound according to the Embodiment 1 wherein G¹ represents CH, G² represents CR^{3b}, G³ represents CR^{3c}, and G⁴ represents CH.
[Embodiment 23] The compound according to the Embodiment 2 wherein G¹ represents CH, G² represents CR^{3b}, G³ represents CR^{3c}, and G⁴ represents CH.
[Embodiment 24] The compound according to the Embodiment 3 wherein G¹ represents CH, G² represents CR^{3b}, G³ represents CR^{3c}, and G⁴ represents CH.
[Embodiment 25] The compound according to the Embodiment 4 wherein G¹ represents CH, G² represents CR^{3b}, G³ represents CR^{3c}, and G⁴ represents CH.
[Embodiment 26] The compound according to the Embodiment 5 wherein G¹ represents CH, G² represents CR^{3b}, G³ represents CR^{3c}, and G⁴ represents CH.
[Embodiment 27] The compound according to the Embodiment 1 wherein G¹ represents a nitrogen atom, G² represents CR^{3b}, G³ represents CR^{3c}, and G⁴ represents CH.
[Embodiment 28] The compound according to the Embodiment 2 wherein G¹ represents a nitrogen atom, G² represents CR^{3b}, G³ represents CR^{3c}, and G⁴ represents CH.
[Embodiment 29] The compound according to the Embodiment 3 wherein G¹ represents a nitrogen atom, G² represents CR^{3b}, G³ represents CR^{3c}, and G⁴ represents CH.
[Embodiment 30] The compound according to the Embodiment 4 wherein G¹ represents a nitrogen atom, G² represents CR^{3b}, G³ represents CR^{3c}, and G⁴ represents CH.
[Embodiment 31] The compound according to the Embodiment 5 wherein G¹ represents a nitrogen atom, G² represents CR^{3b}, G³ represents CR^{3c}, and G⁴ represents CH.
[Embodiment 32] The compound according to the Embodiment 1 wherein G¹ represents a nitrogen atom, G² represents CR^{3b}, G³ represents CR^{3c}, and G⁴ represents CH.
[Embodiment 33] The compound according to the Embodiment 2 wherein G¹ represents CH, G² represents CR^{3b}, G³ represents CR^{3c}, and G⁴ represents a nitrogen atom.
[Embodiment 34] The compound according to the Embodiment 3 wherein G¹ represents CH, G² represents CR^{3b}, G³ represents CR^{3c}, and G⁴ represents a nitrogen atom.
[Embodiment 35] The compound according to the Embodiment 4 wherein G¹ represents CH, G² represents CR^{3b}, G³ represents CR^{3c}, and G⁴ represents a nitrogen atom.
[Embodiment 36] The compound according to the Embodiment 5 wherein G¹ represents CH, G² represents CR^{3b}, G³ represents CR^{3c}, and G⁴ represents a nitrogen atom.
[Embodiment 37] The present compound N wherein R² represents a C1-C6 alkyl group.
[Embodiment 38] The present compound N wherein R² represents an ethyl group.
[Embodiment 39] The compound according to the Embodiment 1 wherein R² represents a C1-C6 alkyl group.
[Embodiment 40] The compound according to the Embodiment 2 wherein R² represents a C1-C6 alkyl group.
[Embodiment 41] The compound according to the Embodiment 3 wherein R² represents a C1-C6 alkyl group.
[Embodiment 42] The compound according to the Embodiment 4 wherein R² represents a C1-C6 alkyl group.
[Embodiment 43] The compound according to the Embodiment 5 wherein R² represents a C1-C6 alkyl group.
[Embodiment 44] The compound according to the Embodiment 6 wherein R² represents a C1-C6 alkyl group.
[Embodiment 45] The compound according to the Embodiment 7 wherein R² represents a C1-C6 alkyl group.
[Embodiment 46] The compound according to the Embodiment 8 wherein R² represents a C1-C6 alkyl group.
[Embodiment 47] The compound according to the Embodiment 9 wherein R² represents a C1-C6 alkyl group.
[Embodiment 48] The compound according to the Embodiment 10 wherein R² represents a C1-C6 alkyl group.
[Embodiment 49] The compound according to the Embodiment 11 wherein R² represents a C1-C6 alkyl group.
[Embodiment 50] The compound according to the Embodiment 12 wherein R² represents a C1-C6 alkyl group.
[Embodiment 51] The compound according to the Embodiment 13 wherein R² represents a C1-C6 alkyl group.
[Embodiment 52] The compound according to the Embodiment 14 wherein R² represents a C1-C6 alkyl group.
[Embodiment 53] The compound according to the Embodiment 15 wherein R² represents a C1-C6 alkyl group.
[Embodiment 54] The compound according to the Embodiment 16 wherein R² represents a C1-C6 alkyl group.
[Embodiment 55] The compound according to the Embodiment 17 wherein R² represents a C1-C6 alkyl group.
[Embodiment 56] The compound according to the Embodiment 18 wherein R² represents a C1-C6 alkyl group.
[Embodiment 57] The compound according to the Embodiment 19 wherein R² represents a C1-C6 alkyl group.
[Embodiment 58] The compound according to the Embodiment 20 wherein R² represents a C1-C6 alkyl group.
[Embodiment 59] The compound according to the Embodiment 21 wherein R² represents a C1-C6 alkyl group.
[Embodiment 60] The compound according to the Embodiment 22 wherein R² represents a C1-C6 alkyl group.
[Embodiment 61] The compound according to the Embodiment 23 wherein R² represents a C1-C6 alkyl group.
[Embodiment 62] The compound according to the Embodiment 24 wherein R² represents a C1-C6 alkyl group.
[Embodiment 63] The compound according to the Embodiment 25 wherein R² represents a C1-C6 alkyl group.
[Embodiment 64] The compound according to the Embodiment 26 wherein R² represents a C1-C6 alkyl group.
[Embodiment 65] The compound according to the Embodiment 27 wherein R² represents a C1-C6 alkyl group.
[Embodiment 66] The compound according to the Embodiment 28 wherein R² represents a C1-C6 alkyl group.
[Embodiment 67] The compound according to the Embodiment 29 wherein R² represents a C1-C6 alkyl group.
[Embodiment 68] The compound according to the Embodiment 30 wherein R² represents a C1-C6 alkyl group.
[Embodiment 69] The compound according to the Embodiment 31 wherein R² represents a C1-C6 alkyl group.
[Embodiment 70] The compound according to the Embodiment 32 wherein R² represents a C1-C6 alkyl group.
[Embodiment 71] The compound according to the Embodiment 33 wherein R² represents a C1-C6 alkyl group.
[Embodiment 72] The compound according to the Embodiment 34 wherein R² represents a C1-C6 alkyl group.
[Embodiment 73] The compound according to the Embodiment 35 wherein R² represents a C1-C6 alkyl group.
[Embodiment 74] The compound according to the Embodiment 36 wherein R² represents a C1-C6 alkyl group.
[Embodiment 75] The compound according to the Embodiment 1 wherein R² represents an ethyl group.
[Embodiment 76] The compound according to the Embodiment 2 wherein R² represents an ethyl group.
[Embodiment 77] The compound according to the Embodiment 3 wherein R² represents an ethyl group.
[Embodiment 78] The compound according to the Embodiment 4 wherein R² represents an ethyl group.
[Embodiment 79] The compound according to the Embodiment 5 wherein R² represents an ethyl group.
[Embodiment 80] The compound according to the Embodiment 6 wherein R² represents an ethyl group.
[Embodiment 81] The compound according to the Embodiment 7 wherein R² represents an ethyl group.
[Embodiment 82] The compound according to the Embodiment 8 wherein R² represents an ethyl group.
[Embodiment 83] The compound according to the Embodiment 9 wherein R² represents an ethyl group.
[Embodiment 84] The compound according to the Embodiment 10 wherein R² represents an ethyl group.
[Embodiment 85] The compound according to the Embodiment 11 wherein R² represents an ethyl group.
[Embodiment 86] The compound according to the Embodiment 12 wherein R² represents an ethyl group.
[Embodiment 87] The compound according to the Embodiment 13 wherein R² represents an ethyl group.
[Embodiment 88] The compound according to the Embodiment 14 wherein R² represents an ethyl group.
[Embodiment 89] The compound according to the Embodiment 15 wherein R² represents an ethyl group.
[Embodiment 90] The compound according to the Embodiment 16 wherein R² represents an ethyl group.
[Embodiment 91] The compound according to the Embodiment 17 wherein R² represents an ethyl group.
[Embodiment 92] The compound according to the Embodiment 18 wherein R² represents an ethyl group.
[Embodiment 93] The compound according to the Embodiment 19 wherein R² represents an ethyl group.
[Embodiment 94] The compound according to the Embodiment 20 wherein R² represents an ethyl group.
[Embodiment 95] The compound according to the Embodiment 21 wherein R² represents an ethyl group.
[Embodiment 96] The compound according to the Embodiment 22 wherein R² represents an ethyl group.
[Embodiment 97] The compound according to the Embodiment 23 wherein R² represents an ethyl group.
[Embodiment 98] The compound according to the Embodiment 24 wherein R² represents an ethyl group.
[Embodiment 99] The compound according to the Embodiment 25 wherein R² represents an ethyl group.
[Embodiment 100] The compound according to the Embodiment 26 wherein R² represents an ethyl group.
[Embodiment 101] The compound according to the Embodiment 27 wherein R² represents an ethyl group.
[Embodiment 102] The compound according to the Embodiment 28 wherein R² represents an ethyl group.
[Embodiment 103] The compound according to the Embodiment 29 wherein R² represents an ethyl group.
[Embodiment 104] The compound according to the Embodiment 30 wherein R² represents an ethyl group.
[Embodiment 105] The compound according to the Embodiment 31 wherein R² represents an ethyl group.
[Embodiment 106] The compound according to the Embodiment 32 wherein R² represents an ethyl group.
[Embodiment 107] The compound according to the Embodiment 33 wherein R² represents an ethyl group.
[Embodiment 108] The compound according to the Embodiment 34 wherein R² represents an ethyl group.
[Embodiment 109] The compound according to the Embodiment 35 wherein R² represents an ethyl group.
[Embodiment 110] The compound according to the Embodiment 36 wherein R² represents an ethyl group.
[Embodiment 111] The compound according to any one of the Embodiment 1 to the Embodiment 110 or the present compound N, wherein R¹ represents a C1-C6 alkyl group having one or more substituents selected from a group consisting of halogen atom and cyano group; a cyclopropyl group optionally having one or more substituents selected from the group consisting of cyano group and halogen atom; S(O)ₘR⁸; or OR⁸, and R⁸ represents a C1-C6 alkyl group having one or more substituents selected from a group consisting of halogen atom and cyano group.
[Embodiment 112] The compound according to the Embodiment 111 wherein A¹ represents CR^{4a}, A² represents a nitrogen atom, R^{4a} represents a C1-C6 alkyl group optionally having one or more halogen atoms, a halogen atom or a hydrogen atom, and R^{6a}, R^{6b}, R^{6c} and R^{6d} are identical to or different from each other and each represents a halogen atom or a hydrogen atom.
[Embodiment 113] The compound according to the Embodiment 111 wherein R^{4a} represents a C1-C6 alkyl group optionally having one or more halogen atoms, a halogen atom or a hydrogen atom, B¹ represents CR^{6a}, and a combination of B², B³ and B⁴ represents a combination in which B² represents CR¹, B³ represents CR^{6c}, and B⁴ represents a nitrogen atom or CR^{6d}; a combination in which B² represents CR⁶b, B³ represents CR¹, and B⁴ represents a nitrogen atom or CR^{6d}; or a combination in which B² represents CR^{6b}, B³ represents CR^{6c}, and B⁴ represents CR¹, and R^{6a}, R^{6b}, R^{6c} and R^{6d} are identical to or different from each other and each represents a halogen atom or a hydrogen atom.
[Embodiment 114] The compound according to the Embodiment 113 wherein A¹ represents CR^{4a}, and A² represents a nitrogen atom.
[Embodiment 115] The compound according to the Embodiment 111 wherein A¹ represents CH, A² represents a nitrogen atom, B¹ represents CH, a combination of B², B³ and B⁴ represents a combination in which B² represents CR¹, B³ represents CH, and B⁴ represents a nitrogen atom or CH; a combination in which B² represents CH, B³ represents CR¹, and B⁴ represents a nitrogen atom or CH; or a combination in which B² represents CH, B³ represents CH, and B⁴ represents CR¹.
[Embodiment 116] The compound according to the Embodiment 111 wherein R^{4a} and R^{4b} represent a hydrogen atom, B¹ represents CH, and a combination of B², B³ and B⁴ represents a combination in which B² represents CR¹, B³ represents CH, and B⁴ represents CH; a combination in which B² represents CH, B³ represents CR¹, and B⁴ represents CH; or a combination in which B² represents CH, B³ represents CH, and B⁴ represents CR¹.
[Embodiment 117] The compound according to the Embodiment 111 wherein A¹ represents CH, A² represents a nitrogen atom, B¹ represents CH, and a combination of B², B³ and B⁴ represents a combination in which B² represents CR¹, B³ represents CH, and B⁴ represents CH; a combination in which B² represents CH, B³ represents CR¹, and B⁴ represents CH; or a combination in which B² represents CH, B³ represents CH, and B⁴ represents CR¹.
[Embodiment 118] The compound according to any one of the Embodiment 1 to the Embodiment 110 or the present compound N, wherein B¹ represents CH, a combination of B², B³ and B⁴ represents a combination in which B² represents CR¹, B³ represents CH, and B⁴ represents a nitrogen atom or CH; a combination in which B² represents CH, B³ represents CR¹, and B⁴ represents a nitrogen atom or CH; or a combination in which B² represents CH, B³ represents CH, and B⁴ represents CR¹, R¹ represents a C1-C3 fluoroalkyl group or S(O)ₘCF₃, and R^{4a} and R^{4b} represent a hydrogen atom.
[Embodiment 119] The compound according to any one of the Embodiment 1 to the Embodiment 110 or the present compound N, wherein B¹ represents CH, a combination of B², B³ and B⁴ represents a combination in which B² represents CR¹, B³ represents CH, and B⁴ represents CH; a combination in which B² represents CH, B³ represents CR¹, and B⁴ represents CH; or a combination in which B² represents CH, B³ represents CH, B⁴ represents CR¹, and R¹ represents a C1-C3 fluoroalkyl group or S(O)ₘCF₃, and R^{4a} and R^{4b} represent a hydrogen atom.
[Embodiment 120] The compound according to any one of the Embodiment 1 to the Embodiment 110 or the present compound N, wherein B¹ represents CH, a combination of B², B³ and B⁴ represents a combination in which B² represents CR¹, B³ represents CH, and B⁴ represents nitrogen atom or CH; a combination in which B² represents CH, B³ represents CH, and B⁴ represents CR¹, R¹ represents a C1-C3 fluoroalkyl group or S(O)ₘCF₃, A¹ represents CH, and A² represents a nitrogen atom.
[Embodiment 121] The compound according to any one of the Embodiment 1 to the Embodiment 110 or the present compound N, wherein B¹ represents CH, a combination of B², B³ and B⁴ represents a combination in which B² represents CR¹, B³ represents CH, and B⁴ represents CH; a combination in which B² represents CH, B³ represents CR¹, and B⁴ represents CH; or a combination in which B² represents CH, B³ represents CH, and B⁴ represents CR¹, R¹ represents a C1-C3 fluoroalkyl group or S(O)ₘCF₃, A¹ represents CH, and A² represents a nitrogen atom.
[Embodiment 122] The compound according to any one of the Embodiment 1 to the Embodiment 110 or the present compound N, wherein B¹ represents CH, B⁴ represents a nitrogen atom, a combination of B² and B³ represents a combination in which B² represents CR¹, and B³ represents CH; or a combination in which B² represents CH, and B³ represents CR¹, R¹ represents a C1-C3 fluoroalkyl group or S(O)ₘCF₃, A¹ represents CH, and A² represents a nitrogen atom.
[Embodiment 123] The compound according to any one of the Embodiment 1 to the Embodiment 110 or the present compound N, wherein R¹ represents a C1-C3 fluoroalkyl group, A¹ represents CH, A² represents a nitrogen atom, and R^{6a}, R^{6b}, R^{6c} and R^{6d} represent a hydrogen atom.
[Embodiment 124] The compound according to any one of the Embodiment 1 to the Embodiment 110 or the present compound N, wherein B¹ represents CH, a combination of B², B³ and B⁴ represents a combination in which B² represents CR¹, B³ represents CH, and B⁴ represents a nitrogen atom or CH; a combination in which B² represents CH, B³ represents CR¹, and B⁴ represents a nitrogen atom or CH; or a combination in which B² represents CH, B³ represents CH, and B⁴ represents CR¹, R¹ represents a C1-C3 fluoroalkyl group, and R^{4a} and R^{4b} represent a hydrogen atom.
[Embodiment 125] The compound according to any one of the Embodiment 1 to the Embodiment 110 or the present compound N, wherein B¹ represents CH, a combination of B², B³ and B⁴ represents a combination in which B² represents CR¹, B³ represents CH, and B⁴ represents a nitrogen atom or CH; a combination in which B² represents CH, B³ represents CR¹, and B⁴ represents a nitrogen atom or CH; or a combination in which B² represents CH, B³ represents CH, and B⁴ represents CR¹, R¹ represents a C1-C3 fluoroalkyl group, A¹ represents CH, and A² represents a nitrogen atom.
[Embodiment 126] The compound according to any one of the Embodiment 1 to the Embodiment 110 or the present compound N, wherein B¹ represents CH, a combination of B², B³ and B⁴ represents a combination in which B² represents CR¹, B³ represents CH, and B⁴ represents CH; a combination in which B² represents CH, B³ represents CR¹, and B⁴ represents CH; or a combination in which B² represents CH, B³ represents CH, and B⁴ represents CR¹, R¹ represents a C1-C3 fluoroalkyl group, and R^{4a} and R^{4b} represent a hydrogen atom.
[Embodiment 127] The compound according to any one of the Embodiment 1 to the Embodiment 110 or the present compound N, wherein B¹ represents CH, B⁴ represents a nitrogen atom, a combination of B² and B³ represents a combination in which B² represents CR¹, and B³ represents CH; or a combination in which B² represents CH, and B³ represents CR¹, R¹ represents a C1-C3 fluoroalkyl group, A¹ represents CH, and A² represents a nitrogen atom.
[Embodiment 128] The compound according to any one of the Embodiment 1 to the Embodiment 110 or the present compound N, wherein B¹ represents a CH, a combination of B², B³ and B⁴ represents a combination in which B² represents CR¹, B³ represents CH, and B⁴ represents CH; a combination of B² represents CH, B³ represents CR¹, and B⁴ represents CH; or a combination in which B² represents CH, B³ represents CH, and B⁴ represents CR¹, R¹ represents a C1-C3 fluoroalkyl group, A¹ represents CH, and A² represents a nitrogen atom.
[Embodiment 129] The compound according to any one of the Embodiment 1 to the Embodiment 110 or the present compound N, wherein Z represents an oxygen atom.
[Embodiment 130] The compound according to the Embodiment 111 wherein Z represents an oxygen atom.
[Embodiment 131] The compound according to the Embodiment 112 wherein Z represents an oxygen atom.
[Embodiment 132] The compound according to the Embodiment 113 wherein Z represents an oxygen atom.
[Embodiment 133] The compound according to the Embodiment 114 wherein Z represents an oxygen atom.
[Embodiment 134] The compound according to the Embodiment 115 wherein Z represents an oxygen atom.
[Embodiment 135] The compound according to the Embodiment 116 wherein Z represents an oxygen atom.
[Embodiment 136] The compound according to the Embodiment 117 wherein Z represents an oxygen atom.
[Embodiment 137] The compound according to the Embodiment 118 wherein Z represents an oxygen atom.
[Embodiment 138] The compound according to the Embodiment 119 wherein Z represents an oxygen atom.
[Embodiment 139] The compound according to the Embodiment 120 wherein Z represents an oxygen atom.
[Embodiment 140] The compound according to the Embodiment 121 wherein Z represents an oxygen atom.
[Embodiment 141] The compound according to the Embodiment 122 wherein Z represents an oxygen atom.
[Embodiment 142] The compound according to the Embodiment 123 wherein Z represents an oxygen atom.
[Embodiment 143] The compound according to the Embodiment 124 wherein Z represents an oxygen atom.
[Embodiment 144] The compound according to the Embodiment 125 wherein Z represents an oxygen atom.
[Embodiment 145] The compound according to the Embodiment 126 wherein Z represents an oxygen atom.
[Embodiment 146] The compound according to the Embodiment 127 wherein Z represents an oxygen atom.
[Embodiment 147] The compound according to the Embodiment 128 wherein Z represents an oxygen atom.
[Embodiment 148] The present compound N wherein Z represents an oxygen atom, A¹ represents CH, A² represents a nitrogen atom, B¹ represents CH, a combination of B², B³ and B⁴ represents a combination in which B² represents CR¹, B³ represents CH, and B⁴ represents a nitrogen atom or CH; a combination in which B² represents CH, B³ represents CR¹, and B⁴ represents a nitrogen atom or CH; or a combination in which B² represents CH, B³ represents CH, and B⁴ represents CR¹, R¹ represents a trifluoromethyl group, R² represents an ethyl group, G¹ represents CH, G² represents CR^{3b}, G³ represents CR^{3c}, G⁴ represents CH, R^{3b} and R^{3c} are identical to or different from each other and each represents a cyclopropyl group, a halogen atom, or a hydrogen atom, and R^{3d} represents a hydrogen atom.

Examples of the Embodiment of the present compound P include the followings.

[Embodiment P1] The present compound P wherein R^{3a} represents a hydrogen atom, R^{3b}, R^{3c} and R^{3d} are identical to or different from each other and each represents a C1-C6 alkyl group, a C2-C6 alkenyl group, a C3-C7 cycloalkyl group {the C1-C6 alkyl group, the C2-C6 alkenyl group, and the C3-C7 cycloalkyl group each may have optionally one or more substituents selected from the group consisting of halogen atom and cyano group}, a phenyl group, a triazolyl group, a pyridyl group, a pyrimidinyl group {the phenyl group, the triazolyl group, the pyridyl group, and the pyrimidinyl group each may have optionally one or more substituents selected from Group J}, OR¹², a hydrogen atom, or halogen atom, when Q represents a group represented by formula Q1, R^{3b} and R^{3d} combined together with two carbon atoms to which they are attached may form a benzene ring {the benzene ring may have optionally one or more substituents selected from a group consisting of C1-C6 alkyl group optionally having one or more halogen atoms, and halogen atom}.
[Embodiment P2] The present compound P wherein R^{3a} and R^{3d} represent a hydrogen atom, R^{3b} and R^{3c} are identical to or different from each other and each represents a C1-C6 alkyl group optionally having one or more halogen atoms, a cyclopropyl group, a hydrogen atom, or a halogen atom, when Q represents a group represented by formula Q1, R^{3b} and R^{3d} combined together with two carbon atoms to which they are attached represent a benzene ring {the benzene ring may have optionally one or more substituents selected from a group consisting of C1-C6 alkyl group optionally having one or more halogen atoms, and halogen atom}.
[Embodiment P3] The present compound P wherein Q represents a group represented by formula Q2.
[Embodiment P4] The compound according to the Embodiment P1 wherein Q represents a group represented by formula Q2.
[Embodiment P5] The compound according to the Embodiment P2 wherein Q represents a group represented by formula Q2.
[Embodiment P6] The present compound P wherein Q represents a group represented by formula Q1.
[Embodiment P7] The compound according to the Embodiment P1 wherein Q represents a group represented by formula Q1.
[Embodiment P8] The compound according to the Embodiment P2 wherein Q represents a group represented by formula Q1.
[Embodiment P9] The present compound P wherein Q represents a group represented by formula Q1, R^{3a} represents a hydrogen atom, R^{3b} and R^{3d} are identical to or different from each other and each represents a C1-C6 alkyl group, a C2-C6 alkenyl group, a C3-C7 cycloalkyl group {the C1-C6 alkyl group, the C2-C6 alkenyl group, and the C3-C7 cycloalkyl group each may have optionally one or more substituents selected from the group consisting of halogen atom and cyano group}, a phenyl group, a triazolyl group, a pyridyl group, a pyrimidinyl group {the phenyl group, the triazolyl group, the pyridyl group, and the pyrimidinyl group each may have optionally one or more substituents selected from Group J}, OR¹², CR³⁰═NOR¹⁷, a hydrogen atom, or a halogen atom.
[Embodiment P10] The present compound P wherein Q represents a group represented by formula Q1, R^{3a} and R^{3d} represent a hydrogen atom, R^{3b} represents a C1-C6 alkyl group optionally having one or more halogen atoms, a cyclopropyl group, a hydrogen atom, or a halogen atom.
[Embodiment P11] The present compound P wherein Q represents a group represented by formula Q1, R^{3a} represents a hydrogen atom, R^{3b} and R^{3d} combined together with two carbon atoms to which they are attached form a benzene ring, a pyrrole ring, a furan ring, a thiophene ring, a pyrazole ring, an imidazole ring, an oxazole ring, an isoxazole ring, a thiazole ring, an isothiazole ring, an oxadiazole ring, a thiadiazole ring, a pyridine ring, a pyridazine ring, a pyrimidine ring, a pyrazine ring {the benzene ring, the pyrrole ring, the furan ring, the thiophene ring, the pyrazole ring, the imidazole ring, the oxazole ring, the isoxazole ring, the thiazole ring, the isothiazole ring, the pyridine ring, the pyridazine ring, the pyrimidine ring, and the pyrazine ring each may have optionally one or more substituents selected from Group H}, or a triazole ring optionally having one or more substituents selected from Group I.
[Embodiment P12] The present compound P wherein Q represents a group represented by formula Q1, R^{3a} represents a hydrogen atom, R^{3b} and R^{3d} combined together with two carbon atoms to which they are attached form a benzene ring {the benzene ring may have optionally one or more substituents selected from a group consisting of C1-C6 alkyl group optionally having one or more halogen atoms, and halogen atom}.
[Embodiment P13] The present compound P wherein G² represents CR^{3b}, G³ represents CR^{3c}, a combination of G¹ and G⁴ represents a combination in which G¹ represents CR^{3a}, and G⁴ represents a nitrogen atom or CR^{3d}; or a combination in which G¹ represents a nitrogen atom, and G⁴ represents CR^{3d}.
[Embodiment P14] The present compound P wherein G¹ represents CH, G² represents CR^{3b}, G³ represents CR^{3c}, and G⁴ represents CH.
[Embodiment P15] The present compound P wherein G¹ represents a nitrogen atom, G² represents CR^{3b}, G³ represents CR^{3c}, and G⁴ represents CH.
[Embodiment P16] The present compound P wherein G¹ represents CH, G² represents CR^{3b}, G³ represents CR^{3c}, and G⁴ represents a nitrogen atom.
[Embodiment P17] The present compound P wherein G² represents CR^{3b}, G³ represents CR^{3c}, a combination of G¹ and G⁴ represents a combination in which G¹ represents CH, and G⁴ represents a nitrogen atom or CR^{3d}; or a combination in which G¹ represents a nitrogen atom, and G⁴ represents CR^{3d}.
[Embodiment P18] The compound according to the Embodiment P2 wherein G² represents CR^{3b}, G³ represents CR^{3c}, a combination of G¹ and G⁴ represents a combination in which G¹ represents CH, and G⁴ represents a nitrogen atom or H; or a combination in which G¹ represents a nitrogen atom, and G⁴ represents CH.
[Embodiment P19] The compound according to the Embodiment P3 wherein G² represents CR^{3b}, G³ represents CR^{3c}, a combination of G¹ and G⁴ represents a combination in which G¹ represents CR^{3a}, and G⁴ represents a nitrogen atom or CR^{3d}; or a combination in which G¹ represents a nitrogen atom, and G⁴ represents CR^{3d}.
[Embodiment P20] The compound according to the Embodiment P4 wherein G² represents CR^{3b}, G³ represents CR^{3c}, a combination of G¹ and G⁴ represents a combination in which G¹ represents CH, and G⁴ represents a nitrogen atom or CR^{3d}; or a combination in which G¹ represents a nitrogen atom, and G⁴ represents CR^{3d}.
[Embodiment P21] The compound according to the Embodiment P5 wherein G² represents CR^{3b}, G³ represents CR^{3c}, a combination of G¹ and G⁴ represents a combination in which G¹ represents CH, and G⁴ represents a nitrogen atom or CH; or a combination in which G¹ represents a nitrogen atom, and G⁴ represents CR^{3d}.
[Embodiment P22] The compound according to the Embodiment P1 wherein G¹ represents CH, G² represents CR^{3b}, G³ represents CR^{3c}, and G⁴ represents CH.
[Embodiment P23] The compound according to the Embodiment P2 wherein G¹ represents CH, G² represents CR^{3b}, G³ represents CR^{3c}, and G⁴ represents CH.
[Embodiment P24] The compound according to the Embodiment P3 wherein G¹ represents CH, G² represents CR^{3b}, G³ represents CR^{3c}, and G⁴ represents CH.
[Embodiment P25] The compound according to the Embodiment P4 wherein G¹ represents CH, G² represents CR^{3b}, G³ represents CR^{3c}, and G⁴ represents CH.
[Embodiment P26] The compound according to the Embodiment P5 wherein G¹ represents CH, G² represents CR^{3b}, G³ represents CR^{3c}, and G⁴ represents CH.
[Embodiment P27] The compound according to the Embodiment P1 wherein G¹ represents a nitrogen atom, G² represents CR^{3b}, G³ represents CR^{3c}, and G⁴ represents CH.
[Embodiment P28] The compound according to the Embodiment P2 wherein G¹ represents a nitrogen atom, G² represents CR^{3b}, G³ represents CR^{3c}, and G⁴ represents CH.
[Embodiment P29] The compound according to the Embodiment P3 wherein G¹ represents a nitrogen atom, G² represents CR^{3b}, G³ represents CR^{3c}, and G⁴ represents CH.
[Embodiment P30] The compound according to the Embodiment P4 wherein G¹ represents a nitrogen atom, G² represents CR^{3b}, G³ represents CR^{3c}, and G⁴ represents CH.
[Embodiment P31] The compound according to the Embodiment P5 wherein G¹ represents a nitrogen atom, G² represents CR^{3b}, G³ represents CR^{3c}, and G⁴ represents CH.
[Embodiment P32] The compound according to the Embodiment P1 wherein G¹ represents CH, G² represents CR^{3b}, G³ represents CR^{3c}, and G⁴ represents a nitrogen atom.
[Embodiment P33] The compound according to the Embodiment P2 wherein G¹ represents CH, G² represents CR^{3b}, G³ represents CR^{3c}, and G⁴ represents a nitrogen atom.
[Embodiment P34] The compound according to the Embodiment P3 wherein G¹ represents CH, G² represents CR^{3b}, G³ represents CR^{3c}, and G⁴ represents a nitrogen atom.
[Embodiment P35] The compound according to the Embodiment P4 wherein G¹ represents CH, G² represents CR^{3b}, G³ represents CR^{3c}, and G⁴ represents a nitrogen atom.
[Embodiment P36] The compound according to the Embodiment P5 wherein G¹ represents CH, G² represents CR^{3b}, G³ represents CR^{3c}, and G⁴ represents a nitrogen atom.
[Embodiment P37] The present compound P wherein R² represents a C1-C6 alkyl group.
[Embodiment P38] The present compound P wherein R² represents an ethyl group.
[Embodiment P39] The compound according to the Embodiment P1 wherein R² represents a C1-C6 alkyl group.
[Embodiment P40] The compound according to the Embodiment P2 wherein R² represents a C1-C6 alkyl group.
[Embodiment P41] The compound according to the Embodiment P3 wherein R² represents a C1-C6 alkyl group.
[Embodiment P42] The compound according to the Embodiment P4 wherein R² represents a C1-C6 alkyl group.
[Embodiment P43] The compound according to the Embodiment P5 wherein R² represents a C1-C6 alkyl group.
[Embodiment P44] The compound according to the Embodiment P6 wherein R² represents a C1-C6 alkyl group.
[Embodiment P45] The compound according to the Embodiment P7 wherein R² represents a C1-C6 alkyl group.
[Embodiment P46] The compound according to the Embodiment P8 wherein R² represents a C1-C6 alkyl group.
[Embodiment P47] The compound according to the Embodiment P9 wherein R² represents a C1-C6 alkyl group.
[Embodiment P48] The compound according to the Embodiment P10 wherein R² represents a C1-C6 alkyl group.
[Embodiment P49] The compound according to the Embodiment P11 wherein R² represents a C1-C6 alkyl group.
[Embodiment P50] The compound according to the Embodiment P12 wherein R² represents a C1-C6 alkyl group.
[Embodiment P51] The compound according to the Embodiment P13 wherein R² represents a C1-C6 alkyl group.
[Embodiment P52] The compound according to the Embodiment P14 wherein R² represents a C1-C6 alkyl group.
[Embodiment P53] The compound according to the Embodiment P15 wherein R² represents a C1-C6 alkyl group.
[Embodiment P54] The compound according to the Embodiment P16 wherein R² represents a C1-C6 alkyl group.
[Embodiment P55] The compound according to the Embodiment P17 wherein R² represents a C1-C6 alkyl group.
[Embodiment P56] The compound according to the Embodiment P18 wherein R² represents a C1-C6 alkyl group.
[Embodiment P57] The compound according to the Embodiment P19 wherein R² represents a C1-C6 alkyl group.
[Embodiment P58] The compound according to the Embodiment P20 wherein R² represents a C1-C6 alkyl group.
[Embodiment P59] The compound according to the Embodiment P21 wherein R² represents a C1-C6 alkyl group.
[Embodiment P60] The compound according to the Embodiment P22 wherein R² represents a C1-C6 alkyl group.
[Embodiment P61] The compound according to the Embodiment P23 wherein R² represents a C1-C6 alkyl group.
[Embodiment P62] The compound according to the Embodiment P24 wherein R² represents a C1-C6 alkyl group.
[Embodiment P63] The compound according to the Embodiment P25 wherein R² represents a C1-C6 alkyl group.
[Embodiment P64] The compound according to the Embodiment P26 wherein R² represents a C1-C6 alkyl group.
[Embodiment P65] The compound according to the Embodiment P27 wherein R² represents a C1-C6 alkyl group.
[Embodiment P66] The compound according to the Embodiment P28 wherein R² represents a C1-C6 alkyl group.
[Embodiment P67] The compound according to the Embodiment P29 wherein R² represents a C1-C6 alkyl group.
[Embodiment P68] The compound according to the Embodiment P30 wherein R² represents a C1-C6 alkyl group.
[Embodiment P69] The compound according to the Embodiment P31 wherein R² represents a C1-C6 alkyl group.
[Embodiment P70] The compound according to the Embodiment P32 wherein R² represents a C1-C6 alkyl group.
[Embodiment P71] The compound according to the Embodiment P33 wherein R² represents a C1-C6 alkyl group.
[Embodiment P72] The compound according to the Embodiment P34 wherein R² represents a C1-C6 alkyl group.
[Embodiment P73] The compound according to the Embodiment P35 wherein R² represents a C1-C6 alkyl group.
[Embodiment P74] The compound according to the Embodiment P36 wherein R² represents a C1-C6 alkyl group.
[Embodiment P75] The compound according to the Embodiment P1 wherein R² represents an ethyl group.
[Embodiment P76] The compound according to the Embodiment P2 wherein R² represents an ethyl group.
[Embodiment P77] The compound according to the Embodiment P3 wherein R² represents an ethyl group.
[Embodiment P78] The compound according to the Embodiment P4 wherein R² represents an ethyl group.
[Embodiment P79] The compound according to the Embodiment P5 wherein R² represents an ethyl group.
[Embodiment P80] The compound according to the Embodiment P6 wherein R² represents an ethyl group.
[Embodiment P81] The compound according to the Embodiment P7 wherein R² represents an ethyl group.
[Embodiment P82] The compound according to the Embodiment P8 wherein R² represents an ethyl group.
[Embodiment P83] The compound according to the Embodiment P9 wherein R² represents an ethyl group.
[Embodiment P84] The compound according to the Embodiment P10 wherein R² represents an ethyl group.
[Embodiment P85] The compound according to the Embodiment P11 wherein R² represents an ethyl group.
[Embodiment P86] The compound according to the Embodiment P12 wherein R² represents an ethyl group.
[Embodiment P87] The compound according to the Embodiment P13 wherein R² represents an ethyl group.
[Embodiment P88] The compound according to the Embodiment P14 wherein R² represents an ethyl group.
[Embodiment P89] The compound according to the Embodiment P15 wherein R² represents an ethyl group.
[Embodiment P90] The compound according to the Embodiment P16 wherein R² represents an ethyl group.
[Embodiment P91] The compound according to the Embodiment P17 wherein R² represents an ethyl group.
[Embodiment P92] The compound according to the Embodiment P18 wherein R² represents an ethyl group.
[Embodiment P93] The compound according to the Embodiment P19 wherein R² represents an ethyl group.
[Embodiment P94] The compound according to the Embodiment P20 wherein R² represents an ethyl group.
[Embodiment P95] The compound according to the Embodiment P21 wherein R² represents an ethyl group.
[Embodiment P96] The compound according to the Embodiment P22 wherein R² represents an ethyl group.
[Embodiment P97] The compound according to the Embodiment P23 wherein R² represents an ethyl group.
[Embodiment P98] The compound according to the Embodiment P24 wherein R² represents an ethyl group.
[Embodiment P99] The compound according to the Embodiment P25 wherein R² represents an ethyl group.
[Embodiment P100] The compound according to the Embodiment P26 wherein R² represents an ethyl group.
[Embodiment P101] The compound according to the Embodiment P27 wherein R² represents an ethyl group.
[Embodiment P102] The compound according to the Embodiment P28 wherein R² represents an ethyl group.
[Embodiment P103] The compound according to the Embodiment P29 wherein R² represents an ethyl group.
[Embodiment P104] The compound according to the Embodiment P30 wherein R² represents an ethyl group.
[Embodiment P105] The compound according to the Embodiment P31 wherein R² represents an ethyl group.
[Embodiment P106] The compound according to the Embodiment P32 wherein R² represents an ethyl group.
[Embodiment P107] The compound according to the Embodiment P33 wherein R² represents an ethyl group.
[Embodiment P108] The compound according to the Embodiment P34 wherein R² represents an ethyl group.
[Embodiment P109] The compound according to the Embodiment P35 wherein R² represents an ethyl group.
[Embodiment P110] The compound according to the Embodiment P36 wherein R² represents an ethyl group.
[Embodiment P111] The compound according to any one of the Embodiment P1 to the Embodiment P110 or the present compound P wherein R¹ represents a C1-C6 alkyl group having one or more substituents selected from a group consisting of halogen atom and cyano group; a cyclopropyl group optionally having one or more substituents selected from the group consisting of cyano group and halogen atom; S(O)ₘR⁸; or OR⁸, R⁸ represents a C1-C6 alkyl group having one or more substituents selected from a group consisting of halogen atom and cyano group.
[Embodiment P112] The compound according to the Embodiment P111 wherein A¹ represents CR^{4a}, A² represents a nitrogen atom, R^{4a} represents a C1-C6 alkyl group optionally having one or more halogen atoms, a halogen atom or a hydrogen atom, R^{6a}, R^{6b}, R^{6c} and R^{6d} are identical to or different from each other and each represents a halogen atom or a hydrogen atom.
[Embodiment P113] The compound according to the Embodiment P111 wherein R^{4a} represents a C1-C6 alkyl group optionally having one or more halogen atoms, B¹ represents CR^{6a}, a combination of B², B³ and B⁴ represents a combination in which B² represents CR¹, B³ represents CR^{6c}, B⁴ represents a nitrogen atom or CR^{6d}; a combination in which B² represents CR^{6b}, B³ represents CR¹, and B⁴ represents a nitrogen atom or CR^{6d}; or a combination in which B² represents CR^{6b}, B³ represents CR^{6c}, and B⁴ represents CR¹, and R^{6a}, R^{6b}, R^{6c} and R^{6d} are identical to or different from each other and each represents a halogen atom or a hydrogen atom.
[Embodiment P114] The compound according to the Embodiment P113 wherein A¹ represents CR^{4a}, and A² represents a nitrogen atom.
[Embodiment P115] The compound according to the Embodiment P111 wherein A¹ represents CH, and A² represents a nitrogen atom, B¹ represents CH, a combination of B², B³ and B⁴ represents a combination in which B² represents CR¹, B³ represents CH, and B⁴ represents a nitrogen atom or CH; a combination in which B² represents CH, B³ represents CR¹, and B⁴ represents a nitrogen atom or CH; a combination in which B² represents CH, B³ represents CH, and B⁴ represents CR¹.
[Embodiment P116] The compound according to the Embodiment P111 wherein R^{4a} and R^{4b} represent a hydrogen atom, B¹ represents CH, a combination of B², B³ and B⁴ represents a combination in which B² represents CR¹, B³ represents CH, and B⁴ represents CH; a combination in which B² represents CH, B³ represents CR¹, and B⁴ represents CH; or a combination in which B² represents CH, B³ represents CH, and B⁴ represents CR¹.
[Embodiment P117] The compound according to the Embodiment P111 wherein A¹ represents CH, A² represents a nitrogen atom, B¹ represents CH, a combination of B², B³ and B⁴ represents a combination in which B² represents CR¹, B³ represents CH, and B⁴ represents CH; a combination in which B² represents CH, B³ represents CR¹, and B⁴ represents CH; or a combination in which B² represents CH, B³ represents CH, and B⁴ represents CR¹.
[Embodiment P118] The compound according to any one of the Embodiment P1 to the Embodiment P110 or the present compound P wherein B¹ represents CH, a combination of B², B³ and B⁴ represents a combination in which B² represents CR¹, B³ represents CH, and B⁴ represents a nitrogen atom or CH; a combination in which B² represents CH, B³ represents CR¹, B⁴ represents a nitrogen atom or CH; or a combination in which B² represents CH, B³ represents CH, and B⁴ represents CR¹, R¹ represents a C1-C3 fluoroalkyl group, and R^{4a} and R^{4b} represents a hydrogen atom.
[Embodiment P119] The compound according to any one of the Embodiment P1 to the Embodiment P110 or the present compound P wherein B¹ represents CH, a combination of B², B³ and B⁴ represents a combination in which B² represents CR¹, B³ represents CH, and B⁴ represents CH; a combination in which B² represents CH, B³ represents CR¹, and B⁴ represents CH; or a combination in which B² represents CH, B³ represents CH, and B⁴ represents CR¹, R¹ represents a C1-C3 fluoroalkyl group, a C1-C3 fluoroalkoxy group, or S(O)ₘCF₃, and R^{4a} and R^{4b} represent a hydrogen atom.
[Embodiment P120] The compound according to any one of the Embodiment P1 to the Embodiment P110 or the present compound P wherein B¹ represents CH, a combination of B², B³ and B⁴ represents a combination in which B² represents CR¹, B³ represents CH, and B⁴ represents a nitrogen atom or CH; a combination in which B² represents CH, B³ represents CR¹, and B⁴ represents a nitrogen atom or CH; a combination in which B² represents CH, B³ represents CH, and B⁴ represents CR¹, R¹ represents a C1-C3 fluoroalkyl group, A¹ represents CH, and A² represents a nitrogen atom.
[Embodiment P121] The compound according to any one of the Embodiment P1 to the Embodiment P110 or the present compound P wherein B¹ represents CH, a combination of B², B³ and B⁴ represents a combination in which B² represents CR¹, B³ represents CH, and B⁴ represents CH; a combination in which B² represents CH, B³ represents CR¹, and B⁴ represents CH; a combination in which B² represents CH, B³ represents CH, and B⁴ represents CR¹, R¹ represents a C1-C3 fluoroalkyl group, a C1-C3 fluoroalkoxy group, or S(O)ₘCF₃, A¹ represents CH, and A² represents a nitrogen atom.
[Embodiment P122] The compound according to any one of the Embodiment P1 to the Embodiment P110 or the present compound P wherein B¹ represents CH, B⁴ represents a nitrogen atom, a combination of B² and B³ represents a combination in which B² represents CR¹, and B³ represents CH; or a combination in which B² represents CH, and B³ represents CR¹, R¹ represents a C1-C3 fluoroalkyl group, A¹ represents CH, and A² represents a nitrogen atom.
[Embodiment P123] The compound according to any one of the Embodiment P1 to the Embodiment P110 or the present compound P wherein R¹ represents a C1-C3 fluoroalkyl group, a C1-C3 fluoroalkoxy group, or S(O)ₘCF₃, A¹ represents CH, A² represents a nitrogen atom, and R^{6a}, R^{6b}, R^{6c} and R^{6d} represent a hydrogen atom.
[Embodiment P124] The compound according to the Embodiment P111 wherein A¹ and A² represent a nitrogen atom, R^{6a}, R^{6b}, R^{6c} and R^{6d} are identical to or different from each other and each represents a halogen atom or a hydrogen atom.
[Embodiment P125] The compound according to the Embodiment P124 wherein B¹ represents CR^{6a}, a combination of B², B³ and B⁴ represents a combination in which B² represents CR¹, B³ represents CR^{6c}, and B⁴ represents a nitrogen atom or CR^{6d}; a combination in which B² represents CR⁶b, and B³ represents CR¹, and B⁴ represents a nitrogen atom or CR^{6d}; or a combination in which B² represents CR^{6b}, B³ represents CR^{6c}, and B⁴ represents CR¹.
[Embodiment P126] The compound according to the Embodiment P124 wherein B¹ represents CH, a combination of B², B³ and B⁴ represents a combination in which B² represents CR¹, B³ represents CH, and B⁴ represents a nitrogen atom or CH; a combination in which B² represents CH, B³ represents CR¹, and B⁴ represents a nitrogen atom or CH; or a combination in which B² represents CH, B³ represents CH, and B⁴ represents CR¹.
[Embodiment P127] The compound according to the Embodiment P124 wherein B¹ represents CH, a combination of B², B³ and B⁴ represents a combination in which B² represents CR¹, B³ represents CH, and B⁴ represents CH; a combination in which B² represents CH, B³ represents CR¹, and B⁴ represents CH; or a combination in which B² represents CH, B³ represents CH, and B⁴ represents CR¹.
[Embodiment P128] The compound according to any one of the Embodiment P1 to the Embodiment P110 or the present compound P wherein A¹ and A² represent a nitrogen atom, B¹ represents CH, a combination of B², B³ and B⁴ represents a combination in which B² represents CR¹, B³ represents CH, and B⁴ represents a nitrogen atom or CH; a combination in which B² represents CH, B³ represents CR¹, and B⁴ represents a nitrogen atom or CH; or a combination in which B² represents CH, B³ represents CH, and B⁴ represents CR¹, and R¹ represents a C1-C3 fluoroalkyl group.
[Embodiment P129] The compound according to any one of the Embodiment P1 to the Embodiment P110 or the present compound P wherein A¹ and A² represent a nitrogen atom, B¹ represents CH, a combination of B², B³ and B⁴ represents a combination in which B² represents CR¹, B³ represents CH, and B⁴ represents CH; a combination in which B² represents CH, B³ represents CR¹, and B⁴ represents CH; or a combination in which B² represents CH, B³ represents CH, and B⁴ represents CR¹, and R¹ represents a C1-C3 fluoroalkyl group, a C1-C3 fluoroalkoxy group, or S(O)ₘCF₃.
[Embodiment P130] The compound according to any one of the Embodiment P1 to the Embodiment P110 or the present compound P wherein A¹ and A² represent a nitrogen atom, B¹ represents CH, B⁴ represents a nitrogen atom, a combination of B² and B³ represents a combination in which B² represents CR¹, and B³ represents CH; or a combination in which B² represents CH, and B³ represents CR¹, and R¹ represents a C1-C3 fluoroalkyl group.
[Embodiment P131] The compound according to any one of the Embodiment P1 to the Embodiment P110 or the present compound P wherein A¹ and A² represent a nitrogen atom, R¹ represents a C1-C3 fluoroalkyl group, a C1-C3 fluoroalkoxy group, or S(O)ₘCF₃, and R^{6a}, R^{6b}, R^{6c} and R^{6d} represent a hydrogen atom.
[Embodiment P132] The compound according to any one of the Embodiment P1 to the Embodiment 110 or the present compound P wherein z represents an oxygen atom.
[Embodiment P133] The compound according to the Embodiment P111 or the present compound P wherein Z represents an oxygen atom.
[Embodiment P134] The compound according to the Embodiment P112 or the present compound P wherein Z represents an oxygen atom.
[Embodiment P135] The compound according to the Embodiment P113 or the present compound P wherein Z represents an oxygen atom.
[Embodiment P136] The compound according to the Embodiment P114 or the present compound P wherein Z represents an oxygen atom.
[Embodiment P137] The compound according to the Embodiment P115 or the present compound P wherein Z represents an oxygen atom.
[Embodiment P138] The compound according to the Embodiment P116 or the present compound P wherein Z represents an oxygen atom.
[Embodiment P139] The compound according to the Embodiment P117 or the present compound P wherein Z represents an oxygen atom.
[Embodiment P140] The compound according to the Embodiment P118 or the present compound P wherein Z represents an oxygen atom.
[Embodiment P141] The compound according to the Embodiment P119 or the present compound P wherein Z represents an oxygen atom.
[Embodiment P142] The compound according to the Embodiment P120 or the present compound P wherein Z represents an oxygen atom.
[Embodiment P143] The compound according to the Embodiment P121 or the present compound P wherein Z represents an oxygen atom.
[Embodiment P144] The compound according to the Embodiment P122 or the present compound P wherein Z represents an oxygen atom.
[Embodiment P145] The compound according to the Embodiment P123 or the present compound P wherein Z represents an oxygen atom.
[Embodiment P146] The compound according to the Embodiment P124 or the present compound P wherein Z represents an oxygen atom.
[Embodiment P147] The compound according to the Embodiment P125 or the present compound P wherein Z represents an oxygen atom.
[Embodiment P148] The compound according to the Embodiment P126 or the present compound P wherein Z represents an oxygen atom.
[Embodiment P149] The compound according to the Embodiment P127 or the present compound P wherein Z represents an oxygen atom.
[Embodiment P150] The compound according to the Embodiment P128 or the present compound P wherein Z represents an oxygen atom.
[Embodiment P151] The compound according to the Embodiment P129 or the present compound P wherein Z represents an oxygen atom.
[Embodiment P152] The compound according to the Embodiment P130 or the present compound P wherein Z represents an oxygen atom.
[Embodiment P153] The compound according to the Embodiment P131 or the present compound P wherein Z represents an oxygen atom.

[Embodiment P2-1] The present compound P wherein Z represents an oxygen atom, B¹ represents CH, a combination of B², B³ and B⁴ represents a combination in which B² represents CR¹, B³ represents CH, and B⁴ represents CH; a combination in which B² represents CH, B³ represents CR¹, B⁴ represents CH; or a combination in which B² represents CH, B³ represents CH, and B⁴ represents CR¹, R¹ represents a C1-C3 chain hydrocarbon group having one or more halogen atoms, S(O)mR⁸, OR⁸, or halogen atom, R⁸ represents a C1-C3 chain hydrocarbon group having one or more halogen atoms.

[Embodiment P2-2] The compound according to the Embodiment P2-1 wherein a combination of B², B³ and B⁴ represents a combination in which B² represents CR¹, B³ represents CH, and B⁴ represents CH; or a combination in which B² represents CH, B³ represents CR¹, and B⁴ represents CH.
[Embodiment P2-3] The compound according to the Embodiment P2-1 wherein A¹ represents CH, and A² represents a nitrogen atom or CH.
[Embodiment P2-4] The compound according to the Embodiment P2-2 wherein A¹ represents CH, and A² represents a nitrogen atom or CH.
[Embodiment P2-5] The compound according to the Embodiment P2-1 wherein A¹ and A² represent a nitrogen atom.
[Embodiment P2-6] The compound according to the Embodiment P2-2 wherein A¹ and A² represent a nitrogen atom.
[Embodiment P2-7] The present compound P wherein R² represents an ethyl group, R^{3a} represents a hydrogen atom, R^{3b}, R^{3c} and R^{3d} are identical to or different from each other and each represents a C1-C3 chain hydrocarbon group optionally having one or more halogen atoms, a C3-C4 cycloalkyl group optionally having one or more halogen atoms, a phenyl group, a pyridyl group, a pyrimidinyl group {the phenyl group, the pyridyl group, and the pyrimidinyl group each may have optionally one or more substituents selected from Group P}, OR¹², a hydrogen atom, or halogen atom, R¹² represents a C1-C3 chain hydrocarbon group optionally having one or more halogen atoms, a C3-C4 cycloalkyl group optionally having one or more halogen atoms, G¹ represents CH, G² represents CR^{3b}, G³ represents CR^{3c}, G⁴ represents CR^{3d} or a nitrogen atom, when Q represents a group represented by formula Q1, R^{3b} and R^{3d} combined together with two carbon atoms to which they are attached may form a benzene ring optionally having one or more substituents selected from Group P,
Group P: a group consisting of a C1-C3 alkyl group optionally having one or more halogen atoms, a C1-C3 alkoxy group optionally having one or more halogen atoms, and a halogen atom.
[Embodiment P2-8] The present compound P wherein R^{3a} and R^{3d} represent a hydrogen atom, R^{3b} and R^{3c} are identical to or different from each other and each represents a C1-C3 chain hydrocarbon group optionally having one or more halogen atoms, a C3-C4 cycloalkyl group optionally having one or more halogen atoms, a phenyl group, a pyridyl group, pyrimidinyl group {the phenyl group, the pyridyl group, and the pyrimidinyl group may have optionally one or more substituents selected from Group P}, OR¹², a hydrogen atom, or halogen atom, R¹² represents a C1-C3 chain hydrocarbon group optionally having one or more halogen atoms, a C3-C4 cycloalkyl group optionally having one or more halogen atoms, G¹ represents CH, G² represents CR^{3b}, G³ represents CR^{3c}, G⁴ represents CH or a nitrogen atom.
[Embodiment P2-9] The compound according to the Embodiment P2-7 wherein Z represents an oxygen atom, B¹ represents CH, a combination of B², B³ and B⁴ represents a combination in which B² represents CR¹, B³ represents CH, and B⁴ represents CH; a combination in which B² represents CH, B³ represents CR¹, and B⁴ represents CH; or a combination in which B² represents CH, B³ represents CH, and B⁴ represents CR¹, R¹ represents a C1-C3 chain hydrocarbon group having one or more halogen atoms, S(O)ₘR⁸, OR⁸, or halogen atom, R⁸ represents a C1-C3 chain hydrocarbon group having one or more halogen atoms.
[Embodiment P2-10] The compound according to the Embodiment P2-8 wherein Z represents an oxygen atom, B¹ represents CH, a combination of B², B³ and B⁴ represents a combination in which B² represents CR¹, B³ represents CH, and B⁴ represents CH; a combination in which B² represents CH, B³ represents CR¹, and B⁴ represents CH; or a combination in which B² represents CH, B³ represents CH, B⁴ represents CR¹, R¹ represents a C1-C3 chain hydrocarbon group having one or more halogen atoms, S(O)ₘR⁸, OR⁸, or halogen atom, and R⁸ represents a C1-C3 chain hydrocarbon group having one or more halogen atoms.
[Embodiment P2-11] The compound according to the Embodiment P2-7 wherein a combination of B², B³ and B⁴ represents a combination in which B² represents CR¹, B³ represents CH, and B⁴ represents CH; or a combination in which B² represents CH, B³ represents CR¹, and B⁴ represents CH.
[Embodiment P2-12] The compound according to the Embodiment P2-8 wherein a combination of B², B³ and B⁴ represents a combination in which B² represents CR¹, B³ represents CH, and B⁴ represents CH; or a combination in which B² represents CH, B³ represents CR¹, and B⁴ represents CH.
[Embodiment P2-13] The compound according to any one of the Embodiment P2-7 wherein A¹ represents CH, and A² represents a nitrogen atom or CH.
[Embodiment P2-14] The compound according to the Embodiment P2-8 wherein A¹ represents CH, and A² represents a nitrogen atom or CH.
[Embodiment P2-15] The compound according to the Embodiment P2-9 wherein A¹ represents CH, and A² represents a nitrogen atom or CH.
[Embodiment P2-16] The compound according to the Embodiment P2-10 wherein A¹ represents CH, and A² represents a nitrogen atom or CH.
[Embodiment P2-17] The compound according to the Embodiment P2-11 wherein A¹ represents CH, and A² represents a nitrogen atom or CH.
[Embodiment P2-18] The compound according to the Embodiment P2-7 wherein A¹ and A² represents a nitrogen atom.
[Embodiment P2-19] The compound according to the Embodiment P2-8 wherein A¹ and A² represents a nitrogen atom.
[Embodiment P2-20] The compound according to the Embodiment P2-9 wherein A¹ and A² represents a nitrogen atom.
[Embodiment P2-21] The compound according to the Embodiment P2-10 wherein A¹ and A² represents a nitrogen atom.
[Embodiment P2-22] The compound according to the Embodiment P2-11 wherein A¹ and A² represents a nitrogen atom.
[Embodiment P2-23] The present compound P or the compound according to any one of the Embodiment P2-1 to the Embodiment P2-22 wherein Q represents a group represented by formula Q1.
[Embodiment P2-24] The present compound P or the compound according to any one of the Embodiment P2-1 to the Embodiment P2-22 wherein Q represents a group represented by formula Q2.
[Embodiment P2-25] The present compound P or the compound according to any one of the Embodiment P2-1 to the Embodiment P2-22 wherein Q represents a group represented by formula Q1, and R² represents an ethyl group.
[Embodiment P2-26] The present compound P or the compound according to any one of the Embodiment P2-1 to the Embodiment P2-22 wherein Q represents a group represented by formula Q2, and R² represents an ethyl group.
[Embodiment P2-27] The present compound P or the compound according to any one of the Embodiment P2-1 to the Embodiment P2-22 wherein Q represents a group represented by formula Q1, R² represents an ethyl group, and n is 2.
[Embodiment P2-28] The present compound P or the compound according to any one of the Embodiment P2-1 to the Embodiment P2-22 wherein Q represents a group represented by formula Q2, R² represents an ethyl group, and n is 2.
[Embodiment P3-1] The present compound P wherein a combination of B², B³ and B⁴ represents a combination in which B² represents CR¹, B³ represents CH, and B⁴ represents CH.
[Embodiment P3-2] The present compound P wherein A¹ represents CH, and A² represents a nitrogen atom or CH.
[Embodiment P3-3] The present compound P wherein A¹ and A² represents a nitrogen atom.

Examples of the Embodiment of the intermediate compound A include the followings.

[Embodiment A1] The intermediate compound A wherein R^{3a} represents a hydrogen atom, R^{3b}, R^{3c} and R^{3d} are identical to or different from each other and each represents a C1-C6 alkyl group, a C2-C6 alkenyl group, a C3-C7 cycloalkyl group {the C1-C6 alkyl group, the C2-C6 alkenyl group, and the C3-C7 cycloalkyl group each may have optionally one or more substituents selected from the group consisting of halogen atom and cyano group}, a phenyl group, a triazolyl group, a pyridyl group, a pyrimidinyl group {the phenyl group, the triazolyl group, the pyridyl group, and the pyrimidinyl group each may have optionally one or more substituents selected from Group J}, OR¹², CR³⁰═NOR¹⁷, a hydrogen atom, or halogen atom, when Q represents a group represented by formula Q1, and R^{3b} and R^{3d} combined together with two carbon atoms to which they are attached may form a benzene ring {the benzene ring may have optionally one or more substituents selected from a group consisting of C1-C6 alkyl group optionally having one or more halogen atoms, and halogen atom}.
[Embodiment A2] The intermediate compound A wherein R^{3a} and R^{3d} represent a hydrogen atom, R^{3b} and R^{3c} are identical to or different from each other and each represents a C1-C6 alkyl group optionally having one or more halogen atoms, a cyclopropyl group, a hydrogen atom, or a halogen atom, when Q represents a group represented by formula Q1, and R^{3b} and R^{3d} combined together with two carbon atoms to which they are attached may form a benzene ring {the benzene ring may have optionally one or more substituents selected from a group consisting of C1-C6 alkyl group optionally having one or more halogen atoms, and halogen atom}.
[Embodiment A3] The compound according to the Embodiment A¹ wherein Q represents a group represented by formula Q2.
[Embodiment A4] The compound according to the Embodiment A² wherein Q represents a group represented by formula Q2.
[Embodiment A5] The compound according to the Embodiment A¹ wherein Q represents a group represented by formula Q1.
[Embodiment A6] The compound according to the Embodiment A² wherein Q represents a group represented by formula Q1.
[Embodiment A7] The intermediate compound A wherein Q represents a group represented by formula Q1, R^{3a} represents a hydrogen atom, R^{3b} and R^{3d} are identical to or different from each other and each represents a C1-C6 alkyl group, a C2-C6 alkenyl group, a C3-C7 cycloalkyl group {the C1-C6 alkyl group, the C2-C6 alkenyl group, and the C3-C7 cycloalkyl group each may have optionally one or more substituents selected from the group consisting of halogen atom and cyano group}, a phenyl group, a pyridyl group, a pyrimidinyl group {the phenyl group, the pyridyl group, and the pyrimidinyl group each may have optionally one or more substituents selected from Group J}.
[Embodiment A8] The intermediate compound A wherein Q represents a group represented by formula Q1, R^{3a} and R^{3d} represent a hydrogen atom, R^{3b} represents a C1-C6 alkyl group optionally having one or more halogen atoms, a cyclopropyl group, a hydrogen atom, or a halogen atom.
[Embodiment 9] The intermediate compound A wherein Q represents a group represented by formula Q1, R^{3a} represents a hydrogen atom, R^{3b} and R^{3d} combined together with two carbon atoms to which they are attached form a benzene ring, a pyrrole ring, a furan ring, a thiophene ring, a pyrazole ring, an imidazole ring, an oxazole ring, an isoxazole ring, a thiazole ring, an isothiazole ring, an oxadiazole ring, a thiadiazole ring, a pyridine ring, a pyridazine ring, a pyrimidine ring, a pyrazine ring {the benzene ring, the pyrrole ring, the furan ring, the thiophene ring, the pyrazole ring, the imidazole ring, the oxazole ring, the isoxazole ring, the thiazole ring, the isothiazole ring, the pyridine ring, the pyridazine ring, the pyrimidine ring, and the pyrazine ring each may have optionally one or more substituents selected from Group H}, or a triazole ring optionally having one or more substituents selected from Group I.
[Embodiment A10] The intermediate compound A wherein Q represents a group represented by formula Q1, R^{3a} represents a hydrogen atom, R^{3b} and R^{3d} combined together with two carbon atoms to which they are attached form a benzene ring {the benzene ring may have optionally one or more substituents selected from a group consisting of C1-C6 alkyl group optionally having one or more halogen atoms, and halogen atom}.
[Embodiment A11] The intermediate compound A wherein G² represents CR^{3b}, G³ represents CR^{3c}, a combination of G¹ and G⁴ represents a combination in which G¹ represents CR^{3a}, and G⁴ represents a nitrogen atom or CR^{3d}; or a combination in which G¹ represents a nitrogen atom, and G⁴ represents CR^{3d}.
[Embodiment A12] The intermediate compound A wherein G¹ represents CH, G² represents CR^{3b}, G³ represents CR^{3c}, and G⁴ represents CH.
[Embodiment A13] The compound according to the Embodiment A¹ wherein G² represents CR^{3b}, G³ represents CR^{3c}, a combination of G¹ and G⁴ represents a combination in which G¹ represents CH, and G⁴ represents a nitrogen atom or CR^{3d}; a combination in which G¹ represents a nitrogen atom, and G⁴ represents CR^{3d}.
[Embodiment A14] The compound according to the Embodiment A² wherein G² represents CR^{3b}, G³ represents CR^{3c}, a combination of G¹ and G⁴ represents a combination in which G¹ represents CH, and G⁴ represents a nitrogen atom or CH; or a combination in which G¹ represents a nitrogen atom, and G⁴ represents CH.
[Embodiment A15] The compound according to the Embodiment A³, wherein G² represents CR^{3b}, G³ represents CR^{3c}, a combination of G¹ and G⁴ represents a combination in which G¹ represents CH, and G⁴ represents a nitrogen atom or CR^{3d}; or a combination in which G¹ represents a nitrogen atom, and G⁴ represents CR^{3d}.
[Embodiment A16] The compound according to the Embodiment A⁴, wherein G² represents CR^{3b}, G³ represents CR^{3c}, a combination of G¹ and G⁴ represents a combination in which G¹ represents CH, and G⁴ represents a nitrogen atom or CH; or a combination in which G¹ represents a nitrogen atom, and G⁴ represents CH.
[Embodiment A17] The compound according to the Embodiment A¹, wherein G¹ represents CH, G² represents CR^{3b}, G³ represents CR^{3c}, and G⁴ represents CH.
[Embodiment A18] The compound according to the Embodiment A², wherein G¹ represents CH, G² represents CR^{3b}, G³ represents CR^{3c}, and G⁴ represents CH.
[Embodiment A19] The compound according to the Embodiment A³, wherein G¹ represents CH, G² represents CR^{3b}, G³ represents CR^{3c}, and G⁴ represents CH.
[Embodiment A20] The compound according to the Embodiment A⁴, wherein G¹ represents CH, G² represents CR^{3b}, G³ represents CR^{3c}, and G⁴ represents CH.
[Embodiment A21] The intermediate compound A, wherein R² represents a C1-C6 alkyl group.
[Embodiment A22] The intermediate compound A wherein R² represents an ethyl group.
[Embodiment A23] The compound according to the Embodiment A¹, wherein R² represents a C1-C6 alkyl group.
[Embodiment A24] The compound according to the Embodiment A², wherein R² represents a C1-C6 alkyl group.
[Embodiment A25] The compound according to the Embodiment A³, wherein R² represents a C1-C6 alkyl group.
[Embodiment A26] The compound according to the Embodiment A⁴, wherein R² represents a C1-C6 alkyl group.
[Embodiment A27] The compound according to the Embodiment A⁵, wherein R² represents a C1-C6 alkyl group.
[Embodiment A28] The compound according to the Embodiment A⁶, wherein R² represents a C1-C6 alkyl group.
[Embodiment A29] The compound according to the Embodiment A⁷, wherein R² represents a C1-C6 alkyl group.
[Embodiment A30] The compound according to the Embodiment A⁸, wherein R² represents a C1-C6 alkyl group.
[Embodiment A31] The compound according to the Embodiment A⁹, wherein R² represents a C1-C6 alkyl group.
[Embodiment A32] The compound according to the Embodiment A¹⁰, wherein R² represents a C1-C6 alkyl group.
[Embodiment A33] The compound according to the Embodiment A11, wherein R² represents a C1-C6 alkyl group.
[Embodiment A34] The compound according to the Embodiment A¹², wherein R² represents a C1-C6 alkyl group.
[Embodiment A35] The compound according to the Embodiment A¹³, wherein R² represents a C1-C6 alkyl group.
[Embodiment A36] The compound according to the Embodiment A¹⁴, wherein R² represents a C1-C6 alkyl group.
[Embodiment A37] The compound according to the Embodiment A¹⁵, wherein R² represents a C1-C6 alkyl group.
[Embodiment A38] The compound according to the Embodiment A¹⁶, wherein R² represents a C1-C6 alkyl group.
[Embodiment A39] The compound according to the Embodiment A¹⁷, wherein R² represents a C1-C6 alkyl group.
[Embodiment A40] The compound according to the Embodiment A¹⁸, wherein R² represents a C1-C6 alkyl group.
[Embodiment A41] The compound according to the Embodiment A¹⁹, wherein R² represents a C1-C6 alkyl group.
[Embodiment A42] The compound according to the Embodiment A²⁰, wherein R² represents a C1-C6 alkyl group.
[Embodiment A43] The compound according to the Embodiment A¹, wherein R² represents an ethyl group.
[Embodiment A44] The compound according to the Embodiment A², wherein R² represents an ethyl group.
[Embodiment A45] The compound according to the Embodiment A³, wherein R² represents an ethyl group.
[Embodiment A46] The compound according to the Embodiment A⁴, wherein R² represents an ethyl group.
[Embodiment A47] The compound according to the Embodiment A⁵, wherein R² represents an ethyl group.
[Embodiment A48] The compound according to the Embodiment A⁶, wherein R² represents an ethyl group.
[Embodiment A49] The compound according to the Embodiment A⁷, wherein R² represents an ethyl group.
[Embodiment A50] The compound according to the Embodiment A⁸, wherein R² represents an ethyl group.
[Embodiment A51] The compound according to the Embodiment A⁹, wherein R² represents an ethyl group.
[Embodiment A52] The compound according to the Embodiment A¹⁰, wherein R² represents an ethyl group.
[Embodiment A53] The compound according to the Embodiment A11, wherein R² represents an ethyl group.
[Embodiment A54] The compound according to the Embodiment A¹², wherein R² represents an ethyl group.
[Embodiment A55] The compound according to the Embodiment A¹³, wherein R² represents an ethyl group.
[Embodiment A56] The compound according to the Embodiment A¹⁴, wherein R² represents an ethyl group.
[Embodiment A57] The compound according to the Embodiment A¹⁵, wherein R² represents an ethyl group.
[Embodiment A58] The compound according to the Embodiment A¹⁶, wherein R² represents an ethyl group.
[Embodiment A59] The compound according to the Embodiment A¹⁷, wherein R² represents an ethyl group.
[Embodiment A60] The compound according to the Embodiment A¹⁸, wherein R² represents an ethyl group.
[Embodiment A61] The compound according to the Embodiment A¹⁹, wherein R² represents an ethyl group.
[Embodiment A62] The compound according to the Embodiment A²⁰, wherein R² represents an ethyl group.
[Embodiment A63] The compound according to any one of the Embodiment A1 to the Embodiment A62 or the intermediate compound A, wherein a combination of B^{2b}, B^{3c} and B^{4d} represents a combination in which B^{2b} represents CR¹, B^{3c} represents CR^{6cc}, and B^{4d} represents a nitrogen atom or CR^{6dd}; a combination in which B^{2b} represents CR^{6bb}, B^{3c} represents CR¹, and B^{4d} represents a nitrogen atom or CR^{6dd}; or a combination in which B^{2b} represents CR^{6bb}, B^{3c} represents CR^{6cc}, and B^{4d} represents CR¹, R^{6bb}, R^{6cc} and R^{6dd} are identical to or different from each other and each represents a halogen atom or a hydrogen atom, R¹ represents a C1-C6 alkyl group having one or more substituents selected from a group consisting of halogen atom and cyano group; a cyclopropyl group optionally having one or more substituents selected from the group consisting of cyano group and halogen atom; S(O)ₘR⁸; or OR⁸, R⁸ represents a C1-C6 alkyl group having one or more substituents selected from a group consisting of halogen atom and cyano group.
[Embodiment A64] The compound according to the Embodiment A¹ to the Embodiment A62 or the intermediate compound A, wherein a combination of B^{2b}, B^{3c} and B^{4d} represents a combination in which B^{2b} represents CR¹, B^{3c} represents CR^{6cc}, and B^{4d} represents a nitrogen atom or CR^{6dd}; a combination in which B^{2b} represents CR^{6bb}, B^{3c} represents CR¹, and B^{4d} represents a nitrogen atom or CR^{6dd}; or a combination in which B^{2b} represents CR^{6bb}, B^{3c} represents CR^{6cc}, and B^{4d} represents CR¹, R^{6bb}, R^{6cc} and R^{6dd} are identical to or different from each other and each represents a halogen atom or a hydrogen atom, and R¹ represents C1-C3 fluoroalkyl group or S(O) mCF₃.
[Embodiment A65] The compound according to any one of the Embodiment A1 to the Embodiment A62 or the intermediate compound A, wherein a combination of B^{2b}, B^{3c} and B^{4d} represents a combination in which B^{2b} represents CR¹, B^{3c} represents CH, and B^{4d} represents CH; a combination in which B^{2b} represents CH, B^{3c} represents CR¹, and B^{4d} represents CH; or a combination in which B^{2b} represents CH, B^{3c} represents CH, and B^{4d} represents CR¹, R¹ represents a C1-C3 fluoroalkyl group or S(O)ₘCF₃.
[Embodiment A66] The compound according to any one of the Embodiment A1 to the Embodiment A62 or the intermediate compound A, wherein a combination of B^{2b}, B^{3c} and B^{4d} represents a combination in which B^{2b} represents CR¹, B^{3c} represents CH, and B^{4d} represents a nitrogen atom or CH; a combination in which B^{2b} represents CH, B^{3c} represents CR¹, and B^{4d} represents a nitrogen atom or CH; or a combination in which B^{2b} represents CH, B^{3c} represents CH, and B^{4d} represents CR¹, R¹ represents a C1-C3 fluoroalkyl group.
[Embodiment A67] The compound according to any one of the Embodiment A1 to the Embodiment A62 or the intermediate compound A, wherein a combination of B^{2b}, B^{3c} and B^{4d} represents a combination in which B^{2b} represents CR¹, B^{3c} represents CH, and B^{4d} represents CH; a combination in which B^{2b} represents CH, B^{3c} represents CR¹, and B^{4d} represents CH; or a combination in which B^{2b} represents CH, B^{3c} represents CH, and B^{4d} represents CR¹, and R¹ represents a C1-C3 fluoroalkyl group.
[Embodiment A68] The compound according to any one of the Embodiment A1 to the Embodiment A62 or the intermediate compound A, wherein B^{4d} represents a nitrogen atom, a combination of B^{3c} and B^{4d} represents a combination in which B^{2b} represents CR¹, and B^{3c} represents CH; or a combination in which B^{2b} represents CH, and B^{3c} represents CR¹, and R¹ represents a C1-C3 fluoroalkyl group.
[Embodiment A69] The intermediate compound A, wherein a combination of B^{2b}, B^{3c} and B^{4d} represents a combination in which B^{2b} represents CR¹, B^{3c} represents CH, and B^{4d} represents a nitrogen atom or CH; a combination in which B^{2b}represents CH, B^{3c} represents CR¹, and B^{4d} represents a nitrogen atom or CH; or a combination in which B^{2b} represents CH, B^{3c} represents CH, and B^{4d} represents CR¹, R¹ represents a trifluoromethyl group, R² represents an ethyl group, G¹ represents CH, G² represents CR^{3b}, G³ represents CR^{3c}, and G⁴ represents CH, R^{3b} and R^{3c} are identical to or different from each other and each represents a cyclopropyl group, a halogen atom or a hydrogen atom, and R^{3d} represents a hydrogen atom.

Examples of the Embodiment of the intermediate compound B include the followings.

[Embodiment B1] The intermediate compound B, wherein a combination of B²¹, B^{3c} and B^{4d} represents a combination in which B^{2b} represents CR¹, B^{3c} represents CR^{6cc}, and B^{4d} represents a nitrogen atom or CR^{6dd}; a combination in which B^{2b} represents CR^{6bb}, B^{3c} represents CR¹, and B^{4d} represents a nitrogen atom or CR^{6dd}; or a combination in which B^{2b} represents CR^{6bb}, B^{3c} represents CR^{6cc}, and B^{4d} represents CR¹, a combination R^{6bb}, R^{6cc} and R^{6dd} are identical to or different from each other and each represents a halogen atom or a hydrogen atom, R¹ represents a C1-C6 alkyl group having one or more substituents selected from a group consisting of halogen atom and cyano group; a cyclopropyl group optionally having one or more substituents selected from the group consisting of cyano group and halogen atom; S(O)ₘR⁸; or OR⁸, R⁸ represents a C1-C6 alkyl group having one or more substituents selected from a group consisting of halogen atom and cyano group, and R^{4aa} represents a halogen atom or a hydrogen atom.

[Embodiment B2] The intermediate compound B wherein a combination of B^{2b}, B^{3c} and B^{4d} represents a combination in which B^{2b} represents CR¹, B^{3c} represents CR^{6cc}, and B^{4d} represents a nitrogen atom or CR^{6dd}; a combination in which B^{2b} represents CR^{6bb}, B^{3c} represents CR¹, and B^{4d} represents a nitrogen atom or CR^{6dd}; a combination in which B^{2b} represents CR^{6bb}, B^{3c} represents CR^{6cc}, and B^{4d} represents CR¹; R^{6bb}, R^{6cc} and R^{6dd} are identical to or different from each other and each represents a halogen atom or a hydrogen atom, R¹ represents a C1-C3 fluoroalkyl group or S(O)ₘCF³, and R^{4aa} represents a halogen atom or a hydrogen atom.
[Embodiment B3] The intermediate compound B wherein a combination of B^{2b}, B^{3c} and B^{4d} represents a combination in which B^{2b} represents CR¹, B^{3c} represents CH, and B^{4d} represents CH; a combination in which B^{2b} represents CH, B^{3c} represents CR¹, and B^{4d} represents CH; or a combination in which B^{2b} represents CH, B^{3c} represents CH, and B^{4d} represents CR¹, and R¹ represents a C1-C3 fluoroalkyl group or S(O)ₘCF₃° [Embodiment B4] The intermediate compound B wherein a combination of B^{2b}, B³c and B^{4d} represents a combination in which B^{2b} represents CR¹, B^{3c} represents CH, and B^{4d} represents a nitrogen atom or CH; a combination in which B^{2b} represents CH, B^{3c} represents CR¹, and B^{4d} represents a nitrogen atom or CH; or a combination in which B^{2b} represents CH, B^{3c} represents CH, and B^{4d} represents CR¹, R¹ represents a C1-C3 fluoroalkyl group, and R^{4aa} represents a hydrogen atom.
[Embodiment B5] The intermediate compound B wherein a combination of B^{2b}, B^{3c} and B^{4d} represents a combination in which B^{2b} represents CR¹, B^{3c} represents CH, and B^{4d} represents CH; a combination in which B^{2b} represents CH, B^{3c} represents CR¹, and B^{4d} represents CH; or a combination in which B^{2b} represents CH, B^{3c} represents CH, and B^{4d} represents CR¹, R¹ represents a C1-C3 fluoroalkyl group, and R^{4aa} represents a hydrogen atom.
[Embodiment B6] The intermediate compound B wherein B^{4d} represents a nitrogen atom, a combination of B^{3c} and B^{4d} represents a combination in which B²¹ represents CR¹, and B^{3c} represents CH; or a combination in which B^{2b} represents CH, and B^{3c} represents CR¹, R¹ represents a C1-C3 fluoroalkyl group, and R^{4aa} represents a hydrogen atom.
[Embodiment B7] The intermediate compound B wherein G² represents CR^{3b}, G³ represents CR^{3c}, a combination of G¹ and G⁴ represents a combination in which G¹ represents CR^{3a}, and G⁴ represents CR^{3d}; or a combination in which G¹ represents a nitrogen atom, and G⁴ represents CR^{3d}.
[Embodiment B8] The intermediate compound B wherein G¹ represents CH, G² represents CR^{3b}, G³ represents CR^{3c}, and G⁴ represents CH.
[Embodiment B9] The compound according to the Embodiment B¹ wherein G² represents CR^{3b}, G³ represents CR^{3c}, a combination of G¹ and G⁴ represents a combination in which G¹ represents CR^{3a}, and G⁴ represents CR^{3d}; or a combination in which G¹ represents a nitrogen atom, and G⁴ represents CR^{3d}.
[Embodiment B10] The compound according to the Embodiment B² wherein G² represents CR^{3b}, G³ represents CR^{3c}, a combination of G¹ and G⁴ represents a combination in which G¹ represents CR^{3a}, and G⁴ represents CR^{3d}; or a combination in which G¹ represents a nitrogen atom, and G⁴ represents CR^{3d}.
[Embodiment B11] The compound according to the Embodiment B³ wherein G² represents CR^{3b}, G³ represents CR^{3c}, a combination of G¹ and G⁴ represents, a combination in which G¹ represents CR^{3a}, and G⁴ represents CR^{3d}; or a combination in which G¹ represents a nitrogen atom, and G⁴ represents CR^{3d}.
[Embodiment B12] The compound according to the Embodiment B⁴ wherein G² represents CR^{3b}, G³ represents CR^{3c}, a combination of G¹ and G⁴ represents a combination in which G¹ represents CR^{3a}, and G⁴ represents CR^{3d}; or a combination in which G¹ represents a nitrogen atom, and G⁴ represents CR^{3d}.
[Embodiment B13] The compound according to the Embodiment B⁵ wherein G² represents CR^{3b}, G³ represents CR^{3c}, a combination of G¹ and G⁴ represents a combination in which G¹ represents CR^{3a}, and G⁴ represents CR^{3d}; or a combination in which G¹ represents a nitrogen atom, and G⁴ represents CR^{3d}.
[Embodiment B14] The compound according to the Embodiment B⁶ wherein G² represents CR^{3b}, G³ represents CR^{3c}, a combination of G¹ and G⁴ represents a combination in which G¹ represents CR^{3a}, and G⁴ represents CR^{3d}; or a combination in which G¹ represents a nitrogen atom, and G⁴ represents CR^{3d}.
[Embodiment B15] The compound according to the Embodiment B¹ wherein G¹ represents CH, G² represents CR^{3b}, G³ represents CR^{3c}, and G⁴ represents CH.
[Embodiment B16] The compound according to the Embodiment B² wherein G¹ represents CH, G² represents CR^{3b}, G³ represents CR^{3c}, and G⁴ represents CH.
[Embodiment B17] The compound according to the Embodiment B³ wherein G¹ represents CH, G² represents CR^{3b}, G³ represents CR^{3c}, and G⁴ represents CH.
[Embodiment B18] The compound according to the Embodiment B⁴ wherein G¹ represents CH, G² represents CR^{3b}, G³ represents CR^{3c}, and G⁴ represents CH.
[Embodiment B19] The compound according to the Embodiment B⁵ wherein G¹ represents CH, G² represents CR^{3b}, G³ represents CR^{3c}, and G⁴ represents CH.
[Embodiment B20] The compound according to the Embodiment B⁶ wherein G¹ represents CH, G² represents CR^{3b}, G³ represents CR^{3c}, and G⁴ represents CH.
[Embodiment B21] The compound according to any one of the Embodiment B1 to the Embodiment B20 or the intermediate compound B, wherein R^{3a} represents a hydrogen atom, R^{3b}, R^{3c} and R^{3d} are identical to or different from each other and each represents a C1-C6 alkyl group, a C2-C6 alkenyl group, a C3-C7 cycloalkyl group {the C1-C6 alkyl group, the C2-C6 alkenyl group, and the C3-C7 cycloalkyl group each may have optionally one or more substituents selected from the group consisting of halogen atom and cyano group}, a phenyl group, a triazolyl group, a pyridyl group, a pyrimidinyl group {the phenyl group, the triazolyl group, the pyridyl group, and the pyrimidinyl group each may have optionally one or more substituents selected from Group J}, OR¹², CR³⁰═NOR¹⁷, a hydrogen atom, or a halogen atom.
[Embodiment B22] The compound according to any one of the Embodiment B1 to the Embodiment 20 or the intermediate compound B, wherein R^{3a} and R^{3d} represent a hydrogen atom, R^{3b} and R^{3c} are identical to or different from each other and each represents a C1-C6 alkyl group optionally having one or more halogen atoms, a cyclopropyl group, a hydrogen atom, or a halogen atom.
[Embodiment B23] The intermediate compound B, wherein a combination of B^{2b}, B^{3c} and B^{4d} represents a combination in which B^{2b} represents CR¹, B^{3c} represents CH, and B^{4d} represents a nitrogen atom or CH; a combination in which B^{2b} represents CH, B^{3c} represents CR¹, and B^{4d} represents a nitrogen atom or CH; or a combination in which B^{2b} represents CH, B^{3c} represents CH, and B^{4d} represents CR¹, R¹ represents a trifluoromethyl group, G¹ represents CH, G² represents CR^{3b}, G³ represents CR^{3c}, G⁴ represents CH, R^{3b} and R^{3c} are identical to or different from each other and each represents a cyclopropyl group, a halogen atom or a hydrogen atom, R^{3d} represents a hydrogen atom, and R^{4aa} represents a hydrogen atom.
[Embodiment B24] The compound according to the Embodiment B²¹, wherein R³³ represents a halogen atom.
[Embodiment B25] The compound according to the Embodiment B²¹, wherein R³³ represents a hydrogen atom.
[Embodiment B26] The compound according to the Embodiment B²², wherein R³³ represents a halogen atom.
[Embodiment B27] The compound according to the Embodiment B²², wherein R³³ represents a hydrogen atom.
[Embodiment B28] The compound according to the Embodiment B²³, wherein R³³ represents a halogen atom.
[Embodiment B29] The compound according to the Embodiment B²³, wherein R³³ represents a hydrogen atom.

Examples of the Embodiment of the intermediate compound C include the followings.

[Embodiment C1] The intermediate compound C, wherein R^{3a} represents a hydrogen atom, R^{3b}, R^{3c} and R^{3d} are identical to or different from each other and each represents a C1-C6 alkyl group, a C2-C6 alkenyl group, a C3-C7 cycloalkyl group {the C1-C6 alkyl group, the C2-C6 alkenyl group, and the C3-C7 cycloalkyl group each may have optionally one or more substituents selected from the group consisting of halogen atom and cyano group}, a phenyl group, a triazolyl group, a pyridyl group, a pyrimidinyl group {the phenyl group, the triazolyl group, the pyridyl group, and the pyrimidinyl group each may have optionally one or more substituents selected from Group J}, OR¹², CR³⁰═NOR¹⁷, a hydrogen atom, or halogen atom, when Q represents a group represented by formula Q1, R^{3b} and R^{3d} combined together with two carbon atoms to which they are attached may form a benzene ring {the benzene ring may have optionally one or more substituents selected from a group consisting of C1-C6 alkyl group optionally having one or more halogen atoms, and halogen atom}.
[Embodiment C2] The intermediate compound C, wherein R^{3a} and R^{3d} represent a hydrogen atom, R^{3b} and R^{3c} are identical to or different from each other and each represents a C1-C6 alkyl group optionally having one or more halogen atoms, a cyclopropyl group, a hydrogen atom, or halogen atom, when Q represents a group represented by formula Q1, R^{3b} and R^{3d} combined together with two carbon atoms to which they are attached may form a benzene ring {the benzene ring may have optionally one or more substituents selected from a group consisting of C1-C6 alkyl group optionally having one or more halogen atoms, and halogen atom}.
[Embodiment C3] The compound according to the Embodiment C1, wherein Q represents a group represented by formula Q2.
[Embodiment C4] The compound according to the Embodiment C2, wherein Q represents a group represented by formula Q2.
[Embodiment C5] The compound according to the Embodiment C1, wherein Q represents a group represented by formula Q1.
[Embodiment C6] The compound according to the Embodiment C2, wherein Q represents a group represented by formula Q1.
[Embodiment C7] The intermediate compound C, wherein Q represents a group represented by formula Q1, R^{3a} represents a hydrogen atom, R^{3b} and R^{3d} are identical to or different from each other and each represents a C1-C6 alkyl group, a C2-C6 alkenyl group, a C3-C7 cycloalkyl group {the C1-C6 alkyl group, the C2-C6 alkenyl group, and the C3-C7 cycloalkyl group may have optionally one or more substituents selected from the group consisting of halogen atom and cyano group}, a phenyl group, a pyridyl group, a pyrimidinyl group {the phenyl group, the pyridyl group, and the pyrimidinyl group each may have optionally one or more substituents selected from Group J}.
[Embodiment C8] The intermediate compound C, wherein Q represents a group represented by formula Q1, R^{3a} and R^{3d} represent a hydrogen atom, R^{3b} represents a C1-C6 alkyl group optionally having one or more halogen atoms, cyclopropyl group, a hydrogen atom, or a halogen atom.
[Embodiment C9] The intermediate compound C, wherein Q represents a group represented by formula Q1, R^{3a} represents a hydrogen atom, R^{3b} and R^{3d} combined together with two carbon atoms to which they are attached form a benzene ring, a pyrrole ring, a furan ring, a thiophene ring, a pyrazole ring, an imidazole ring, an oxazole ring, an isoxazole ring, a thiazole ring, an isothiazole ring, an oxadiazole ring, a thiadiazole ring, a pyridine ring, a pyridazine ring, a pyrimidine ring, a pyrazine ring {the benzene ring, the pyrrole ring, the furan ring, the thiophene ring, the pyrazole ring, the imidazole ring, the oxazole ring, the isoxazole ring, the thiazole ring, the isothiazole ring, the pyridine ring, the pyridazine ring, the pyrimidine ring, and the pyrazine ring each may have optionally one or more substituents selected from Group H}, or a triazole ring optionally having one or more substituents selected from Group I.
[Embodiment C10] The intermediate compound C, wherein Q represents a group represented by formula Q1, R^{3a} represents a hydrogen atom, R^{3b} and R^{3d} combined together with two carbon atoms to which they are attached form benzene ring {the benzene ring may have optionally one or more substituents selected from a group consisting of C1-C6 alkyl group optionally having one or more halogen atoms, and halogen atom}.
[Embodiment C11] The intermediate compound C, wherein Q represents a group represented by formula Q1, G² represents CR^{3b}, G³ represents CR^{3c}, a combination of G¹ and G⁴ represents a combination in which G¹ represents CR^{3a}, and G⁴ represents a nitrogen atom or CR^{3d}; or a combination in which G¹ represents a nitrogen atom, and G⁴ represents CR^{3d}.
[Embodiment C12] The intermediate compound C, wherein G¹ represents CH, G² represents CR^{3b}, G³ represents CR^{3c}, and G⁴ represents CH.
[Embodiment C13] The compound according to the Embodiment C1, wherein G² represents CR^{3b}, G³ represents CR^{3c}, a combination of G¹ and G⁴ represents a combination in which G¹ represents CH, and G⁴ represents a nitrogen atom or CR^{3d}; or a combination in which G¹ represents a nitrogen atom, and G⁴ represents CR^{3d}.
[Embodiment C14] The compound according to the Embodiment C2, wherein G² represents CR^{3b}, G³ represents CR^{3c}, a combination of G¹ and G⁴ represents a combination in which G¹ represents CH, and G⁴ represents a nitrogen atom or CH; or a combination in which G¹ represents a nitrogen atom, and G⁴ represents CH.
[Embodiment C15] The compound according to the Embodiment C3, wherein G² represents CR^{3b}, G³ represents CR^{3c}, a combination of G¹ and G⁴ represents a combination in which G¹ represents CH, and G⁴ represents a nitrogen atom or CR^{3d}; or a combination in which G¹ represents a nitrogen atom, and G⁴ represents CR^{3d}.
[Embodiment C16] The compound according to the Embodiment C4, wherein G² represents CR^{3b}, G³ represents CR^{3c}, a combination of G¹ and G⁴ represents a combination in which G¹ represents CH, and G⁴ represents a nitrogen atom or CH; or a combination in which G¹ represents a nitrogen atom, and G⁴ represents CH.
[Embodiment C17] The compound according to the Embodiment C1, wherein G¹ represents CH, G² represents CR^{3b}, G³ represents CR^{3c}, and G⁴ represents CH.
[Embodiment C18] The compound according to the Embodiment C2, wherein G¹ represents CH, G² represents CR^{3b}, G³ represents CR^{3c}, and G⁴ represents CH.
[Embodiment C19] The compound according to the Embodiment C3, wherein G¹ represents CH, G² represents CR^{3b}, G³ represents CR^{3c}, and G⁴ represents CH.
[Embodiment C20] The compound according to the Embodiment C4, wherein G¹ represents CH, G² represents CR^{3b}, G³ represents CR^{3c}, and G⁴ represents CH.
[Embodiment C21] The intermediate compound C, wherein R² represents a C1-C6 alkyl group.
[Embodiment C22] The intermediate compound C, wherein R² represents an ethyl group.
[Embodiment C23] The compound according to the Embodiment C1, wherein R² represents a C1-C6 alkyl group.
[Embodiment C24] The compound according to the Embodiment C2, wherein R² represents a C1-C6 alkyl group.
[Embodiment C25] The compound according to the Embodiment C3, wherein R² represents a C1-C6 alkyl group.
[Embodiment C26] The compound according to the Embodiment C4, wherein R² represents a C1-C6 alkyl group.
[Embodiment C27] The compound according to the Embodiment C5, wherein R² represents a C1-C6 alkyl group.
[Embodiment C28] The compound according to the Embodiment C6, wherein R² represents a C1-C6 alkyl group.
[Embodiment C29] The compound according to the Embodiment C7, wherein R² represents a C1-C6 alkyl group.
[Embodiment C30] The compound according to the Embodiment C8, wherein R² represents a C1-C6 alkyl group.
[Embodiment C31] The compound according to the Embodiment C9, wherein R² represents a C1-C6 alkyl group.
[Embodiment C32] The compound according to the Embodiment C10, wherein R² represents a C1-C6 alkyl group.
[Embodiment C33] The compound according to the Embodiment C11, wherein R² represents a C1-C6 alkyl group.
[Embodiment C34] The compound according to the Embodiment C12, wherein R² represents a C1-C6 alkyl group.
[Embodiment C35] The compound according to the Embodiment C13, wherein R² represents a C1-C6 alkyl group.
[Embodiment C36] The compound according to the Embodiment C14, wherein R² represents a C1-C6 alkyl group.
[Embodiment C37] The compound according to the Embodiment C15, wherein R² represents a C1-C6 alkyl group.
[Embodiment C38] The compound according to the Embodiment C16, wherein R² represents a C1-C6 alkyl group.
[Embodiment C39] The compound according to the Embodiment C17, wherein R² represents a C1-C6 alkyl group.
[Embodiment C40] The compound according to the Embodiment C18, wherein R² represents a C1-C6 alkyl group.
[Embodiment C41] The compound according to the Embodiment C19, wherein R² represents a C1-C6 alkyl group.
[Embodiment C42] The compound according to the Embodiment C20, wherein R² represents a C1-C6 alkyl group.
[Embodiment C43] The compound according to the Embodiment C1, wherein R² represents an ethyl group.
[Embodiment C44] The compound according to the Embodiment C2, wherein R² represents an ethyl group.
[Embodiment C45] The compound according to the Embodiment C3, wherein R² represents an ethyl group.
[Embodiment C46] The compound according to the Embodiment C4, wherein R² represents an ethyl group.
[Embodiment C47] The compound according to the Embodiment C5, wherein R² represents an ethyl group.
[Embodiment C48] The compound according to the Embodiment C6, wherein R² represents an ethyl group.
[Embodiment C49] The compound according to the Embodiment C7, wherein R² represents an ethyl group.
[Embodiment C50] The compound according to the Embodiment C8, wherein R² represents an ethyl group.
[Embodiment C51] The compound according to the Embodiment C9, wherein R² represents an ethyl group.
[Embodiment C52] The compound according to the Embodiment C10, wherein R² represents an ethyl group.
[Embodiment C53] The compound according to the Embodiment C11, wherein R² represents an ethyl group.
[Embodiment C54] The compound according to the Embodiment C12, wherein R² represents an ethyl group.
[Embodiment C55] The compound according to the Embodiment C13, wherein R² represents an ethyl group.
[Embodiment C56] The compound according to the Embodiment C14, wherein R² represents an ethyl group.
[Embodiment C57] The compound according to the Embodiment C15, wherein R² represents an ethyl group.
[Embodiment C58] The compound according to the Embodiment C16, wherein R² represents an ethyl group.
[Embodiment C59] The compound according to the Embodiment C17, wherein R² represents an ethyl group.
[Embodiment C60] The compound according to the Embodiment C18, wherein R² represents an ethyl group.
[Embodiment C61] The compound according to the Embodiment C19, wherein R² represents an ethyl group.
[Embodiment C62] The compound according to the Embodiment C20, wherein R² represents an ethyl group.
[Embodiment C63] The compound according to any one of the Embodiment C1 to the Embodiment C62 or the intermediate compound C, wherein a combination of B^{2b}, B^{3c} and B^{4d} represents a combination in which B^{2b} represents CR¹, B^{3c} represents CR^{6cc}, and B^{4d} represents a nitrogen atom or CR^{6dd}; or a combination in which B^{2b} represents CR^{6bb}, B^{3c} represents CR¹, and B^{4d} represents a nitrogen atom or CR^{6dd}; or a combination in which B^{2b} represents CR^{6bb}, B^{3c} represents CR^{6cc}, and B^{4d} represents CR¹, R^{6bb}, R^{6cc} and R^{6dd} are identical to or different from each other and each represents a halogen atom or a hydrogen atom, R¹ represents a C1-C6 alkyl group having one or more substituents selected from a group consisting of halogen atom and cyano group; a cyclopropyl group optionally having one or more substituents selected from the group consisting of cyano group and halogen atom; S(O)ₘR⁸; or OR⁸, R⁸ represents a C1-C6 alkyl group having one or more substituents selected from a group consisting of halogen atom and cyano group.
[Embodiment C64] The compound according to any one of the Embodiment C1 to the Embodiment C62 or the intermediate compound C, wherein a combination of B^{2b}, B^{3c} and B^{4d} represents a combination in which B^{2b} represents CR¹, B^{3c} represents CR^{6cc}, and B^{4d} represents a nitrogen atom or CR^{6dd}; a combination in which B^{2b} represents CR^{6bb}, B^{3c} represents CR¹, and B^{4d} represents a nitrogen atom or CR^{6dd}; or a combination in which B^{2b} represents CR^{6bb}, B^{3c} represents CR^{6cc}, and B^{4d} represents CR¹, R^{6bb}, R^{6cc} and R^{6dd} are identical to or different from each other and each represents a halogen atom or a hydrogen atom, and R¹ represents a C1-C3 fluoroalkyl group, a C1-C3 fluoroalkoxy group, or S(O)ₘCF₃.
[Embodiment C65] The compound according to any one of the Embodiment C1 to the Embodiment C62 or the intermediate compound C, wherein a combination of B^{2b}, B^{3c} and B^{4d} represents a combination in which B^{2b} represents CR¹, B^{3c} represents CH, and B^{4d} represents CH; a combination in which B^{2b} represents CH, B^{3c} represents CR¹, and B^{4d} represents CH; or a combination in which B^{2b} represents CH, B^{3c} represents CH, and B^{4d} represents CR¹, R¹ represents a C1-C3 fluoroalkyl group, a C1-C3 fluoroalkoxy group, or S(O)ₘCF₃.
[Embodiment C66] The compound according to any one of the Embodiment C1 to the Embodiment C62 or the intermediate compound C, wherein a combination of B^{2b}, B^{3c} and B^{4d} represents a combination in which B^{2b} represents CR¹, B^{3c} represents CH, B^{4d} represents a nitrogen atom or CH; a combination in which B^{2b} represents CH, B^{3c} represents CR¹, and B^{4d} represents a nitrogen atom or CH; or a combination in which B^{2b} represents CH, B^{3c} represents CH, and B^{4d} represents CR¹, and R¹ represents a C1-C3 fluoroalkyl group, a C1-C3 fluoroalkoxy group, or S(O)ₘCF³.
[Embodiment C67] The compound according to any one of the Embodiment C1 to the Embodiment C62 or the intermediate compound C, wherein a combination of B^{2b}, B^{3c} and B^{4d} represents a combination in which B^{2b} represents CR¹, B^{3c} represents CH, and B^{4d} represents CH; a combination in which B^{2b} represents CH, B^{3c} represents CR¹, and B^{4d} represents CH; or a combination in which B^{2b} represents CH, B^{3c} represents CH, and B^{4d} represents CR¹, and R¹ represents a C1-C3 fluoroalkyl group, a C1-C3 fluoroalkoxy group, or S(O)ₘCF₃.
[Embodiment C68] The compound according to any one of the Embodiment C1 to the Embodiment C62 or the intermediate compound C, wherein B^{4d} represents a nitrogen atom, a combination of B^{3c} and B^{4d} represents a combination in which B^{2b} represents CR¹, and B^{3c} represents CH; or a combination in which B^{2b} represents CH, and B^{3c} represents CR¹, R¹ represents a C1-C3 fluoroalkyl group, a C1-C3 fluoroalkoxy group, or S(O)ₘCF₃.

Examples of the Embodiment of the intermediate compound D include the followings.

[Embodiment D1] The intermediate compound D, wherein a combination of B^{2b}, B^{3c} and B^{4d} represents a combination in which B^{2b} represents CR¹, B^{3c} represents CR^{6cc}, and B^{4d} represents a nitrogen atom or CR^{6dd}; a combination in which B^{2b} represents CR^{6bb}, B^{3c} represents CR¹, and B^{4d} represents a nitrogen atom or CR^{6dd}; or a combination in which B^{2b} represents CR^{6bb}, B^{3c} represents CR^{6cc}, and B^{4d} represents CR¹, R^{6bb}, R^{6cc} and R^{6dd} are identical to or different from each other and each represents a halogen atom or a hydrogen atom, R¹ represents a C1-C6 alkyl group having one or more substituents selected from a group consisting of halogen atom and cyano group; a cyclopropyl group optionally having one or more substituents selected from the group consisting of cyano group and halogen atom; S(O)ₘR⁸; or OR⁸, R⁸ represents a C1-C6 alkyl group having one or more substituents selected from a group consisting of halogen atom and cyano group, and R^{4aa} represents a halogen atom or a hydrogen atom.
[Embodiment D2] The intermediate compound D, wherein a combination of B^{2b}, B^{3c} and B^{4d} represents a combination in which B^{2b} represents CR¹, B^{3c} represents CR^{6cc}, and B^{4d} represents a nitrogen atom or CR^{6dd}; a combination in which B^{2b} represents CR^{6bb}, B^{3c} represents CR¹, and B^{4d} represents a nitrogen atom or CR^{6dd}; or a combination in which B^{2b} represents CR^{6bb}, B^{3c} represents CR^{6cc}, and B^{4d} represents CR¹, R^{6bb}, R^{6cc} and R^{6dd} are identical to or different from each other and each represents a halogen atom or a hydrogen atom, R¹ represents a C1-C3 fluoroalkyl group, a C1-C3 fluoroalkoxy group, or S(O)ₘCF₃, and R^{4aa} represents a halogen atom or a hydrogen atom.
[Embodiment D3] The intermediate compound D, wherein a combination of B^{2b}, B^{3c} and B^{4d} represents a combination in which B^{2b} represents CR¹, B^{3c} represents CH, and B^{4d} represents CH; a combination in which B^{2b} represents CH, B^{3c} represents CR¹, and B^{4d} represents CH; or a combination in which B^{2b} represents CH, B^{3c} represents CH, and B^{4d} represents CR¹, R¹ represents a C1-C3 fluoroalkyl group, a C1-C3 fluoroalkoxy group, or S(O)ₘCF₃.
[Embodiment D4] The intermediate compound D, wherein a combination of B^{2b}, B^{3c} and B^{4d} represents a combination in which B^{2b} represents CR¹, B^{3c} represents CH, and B^{4d} represents a nitrogen atom or CH; a combination in which B^{2b} represents CH, B^{3c} represents CR¹, and B^{4d} represents a nitrogen atom or CH; or a combination in which B^{2b} represents CH, B^{3c} represents CH, and B^{4d} represents CR¹, R¹ represents a C1-C3 fluoroalkyl group, a C1-C3 fluoroalkoxy group, or S(O)ₘCF₃, and R^{4aa} represents a hydrogen atom.
[Embodiment D5] The intermediate compound D, wherein a combination of B^{2b}, B^{3c} and B^{4d} represents a combination in which B^{2b} represents CR¹, B^{3c} represents CH, and B^{4d} represents CH; a combination in which B^{2b} represents CH, B^{3c} represents CR¹, and B^{4d} represents CH; or a combination in which B^{2b} represents CH, B^{3c} represents CH, and B^{4d} represents CR¹, R¹ represents a C1-C3 fluoroalkyl group, a C1-C3 fluoroalkoxy group, or S(O)ₘCF₃, and R^{4aa} represents a hydrogen atom.
[Embodiment D6] The intermediate compound D, wherein B^{4d} represents a nitrogen atom, a combination of B^{3c} and B^{4d} represents a combination in which B²¹ represents CR¹, and B^{3c} represents CH; or a combination in which B^{2b} represents CH, and B^{3c} represents CR¹, R¹ represents a C1-C3 fluoroalkyl group, a C1-C3 fluoroalkoxy group, or S(O)ₘCF₃, and R^{4aa} represents a hydrogen atom.
[Embodiment D7] The intermediate compound D, wherein G² represents CR^{3b}, G³ represents CR^{3c}, and a combination of G¹ and G⁴ represents, G¹ represents CR^{3a}, and G⁴ represents CR^{3d}; or a combination in which G¹ represents a nitrogen atom, and G⁴ represents CR^{3d}.
[Embodiment D8] The intermediate compound D, wherein G¹ represents CH, G² represents CR^{3b}, G³ represents CR^{3c}, and G⁴ represents CH.
[Embodiment D9] The compound according to the Embodiment D1, wherein G² represents CR^{3b}, G³ represents CR^{3c}, and a combination of G¹ and G⁴ represents a combination in which G¹ represents CR^{3a}, G⁴ represents CR^{3d}; or a combination in which G¹ represents a nitrogen atom, and G⁴ represents CR^{3d}.
[Embodiment D10] The compound according to the Embodiment D2, wherein G² represents CR^{3b}, G³ represents CR^{3c}, and a combination of G¹ and G⁴ represents a combination in which G¹ represents CR^{3a}, and G⁴ represents CR^{3d}; or a combination in which G¹ represents a nitrogen atom, and G⁴ represents CR^{3d}.
[Embodiment D11] The compound according to the Embodiment D3, wherein G² represents CR^{3b}, G³ represents CR^{3c}, and a combination of G¹ and G⁴ represents a combination in which G¹ represents CR^{3a}, and G⁴ represents CR^{3d}; or a combination in G¹ represents a nitrogen atom, and G⁴ represents CR^{3d}.
[Embodiment D12] The compound according to the Embodiment D4, wherein G² represents CR^{3b}, G³ represents CR^{3c}, and a combination of G¹ and G⁴ represents a combination in which G¹ represents CR^{3a}, and G⁴ represents CR^{3d}; or a combination in which G¹ represents a nitrogen atom, and G⁴ represents CR^{3d}.
[Embodiment D13] The compound according to the Embodiment D5, wherein G² represents CR^{3b}, G³ represents CR^{3c}, and a combination of G¹ and G⁴ represents a combination in which G¹ represents CR^{3a}, and G⁴ represents CR^{3d}; or a combination in which G¹ represents a nitrogen atom, and G⁴ represents CR^{3d}.
[Embodiment D14] The compound according to the Embodiment D6, wherein G² represents CR^{3b}, G³ represents CR^{3c}, and a combination of G¹ and G⁴ represents a combination in which G¹ represents CR^{3a}, and G⁴ represents CR^{3d}; or a combination in which G¹ represents a nitrogen atom, and G⁴ represents CR^{3d}.
[Embodiment D15] The compound according to the Embodiment D1, wherein G¹ represents CH, G² represents CR^{3b}, G³ represents CR^{3c}, and G⁴ represents CH.
[Embodiment D16] The compound according to the Embodiment D2, wherein G¹ represents CH, G² represents CR^{3b}, G³ represents CR^{3c}, and G⁴ represents CH.
[Embodiment D17] The compound according to the Embodiment D3, wherein G¹ represents CH, G² represents CR^{3b}, G³ represents CR^{3c}, and G⁴ represents CH.
[Embodiment D18] The compound according to the Embodiment D4, wherein G¹ represents CH, G² represents CR^{3b}, G³ represents CR^{3c}, and G⁴ represents CH.
[Embodiment D19] The compound according to the Embodiment D5, wherein G¹ represents CH, G² represents CR^{3b}, G³ represents CR^{3c}, and G⁴ represents CH.
[Embodiment D20] The compound according to the Embodiment D6, wherein G¹ represents CH, G² represents CR^{3b}, G³ represents CR^{3c}, and G⁴ represents CH.
[Embodiment D21] The compound according to any one of the Embodiment D1 to the Embodiment D20 or the intermediate compound D, wherein R^{3a} represents a hydrogen atom, R^{3b}, R^{3c} and R^{3d} are identical to or different from each other and each represents a C1-C6 alkyl group, a C2-C6 alkenyl group, a C3-C7 cycloalkyl group {the C1-C6 alkyl group, the C2-C6 alkenyl group, and the C3-C7 cycloalkyl group each may have optionally one or more substituents selected from the group consisting of halogen atom and cyano group}, a phenyl group, a triazolyl group, a pyridyl group, a pyrimidinyl group {the phenyl group, the triazolyl group, the pyridyl group, and the pyrimidinyl group each may have optionally one or more substituents selected from Group J}, OR¹², CR³⁰═NOR¹⁷, a hydrogen atom, or a halogen atom.
[Embodiment D22] The compound according to any one of the Embodiment D1 to the Embodiment D20 or the intermediate compound D, wherein R^{3a} and R^{3d} represent a hydrogen atom, R^{3b} and R^{3c} are identical to or different from each other and each represents a C1-C6 alkyl group optionally having one or more halogen atoms, a cyclopropyl group, a hydrogen atom, or a halogen atom.
[Embodiment D23] The intermediate compound D, wherein a combination of B^{2b}, B^{3c} and B^{4d} represents a combination in which B^{2b} represents CR¹, B^{3c} represents CH, B^{4d} represents a nitrogen atom or CH; a combination in which B^{2b} represents CH, B^{3c} represents CR¹, B^{4d} represents a nitrogen atom or CH; or a combination in which B^{2b} represents CH, B^{3c} represents CH, and B^{4d} represents CR¹, R¹ represents a trifluoromethyl group, G¹ represents CH, G² represents CR^{3b}, G³ represents CR^{3c}, and G⁴ represents CH, R^{3b} and R^{3c} are identical to or different from each other and each represents a cyclopropyl group, a halogen atom or a hydrogen atom, R^{3d} represents a hydrogen atom, and R^{4aa} represents a hydrogen atom.
[Embodiment D24] The compound according to the Embodiment D21, wherein R³³ represents a halogen atom.
[Embodiment D25] The compound according to the Embodiment D21, wherein R³³ represents a hydrogen atom.
[Embodiment D26] The compound according to the Embodiment D22, wherein R³³ represents a halogen atom.
[Embodiment D27] The compound according to the Embodiment D22, wherein R³³ represents a hydrogen atom.
[Embodiment D28] The compound according to the Embodiment D23, wherein R³³ represents a halogen atom.
[Embodiment D29] The compound according to the Embodiment D23, wherein R³³ represents a hydrogen atom.

Next, a process for preparing a compound X of the present invention is explained.

### Process 1

A compound represented by formula (I-b) (hereinafter, referred to as compound (I-b)) or a compound represented by formula (I-c) (hereinafter, referred to as compound (I-c)) can be prepared by reacting a compound represented by formula (I-a) (hereinafter, compound (I-a)) with an oxidizing agent.

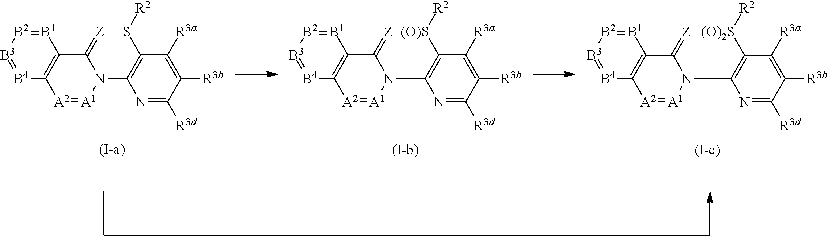

[wherein the symbols are the same as defined above.]

Firstly, a process for the compound (I-b) from the compound (I-a) is described.

The reaction is usually carried out in a solvent. Examples of the solvent to be used in the reaction include halogenated hydrocarbons such as dichloromethane and chloroform (hereinafter, collectively referred to as halogenated hydrocarbons); nitriles such as acetonitrile (hereinafter collectively referred to nitriles); alcohols such as methanol and ethanol (hereinafter, collectively referred to as alcohols); acetic acid; water; and mixed solvents of two or more kinds of the solvents.

Examples of the oxidizing agent to be used in the reaction include sodium periodate, m-chloroperoxybenzoic acid (hereinafter, referred to as mCPBA) and hydrogen peroxide.

When hydrogen peroxide is used as an oxidizing agent, a base or a catalyst may be added as needed.

Examples of the base include sodium carbonate.

When the base is used in the reaction, the base is usually used within a range of 0.01 to 1 molar ration(s), as opposed to 1 mole of the compound (I-a).

Examples of the catalyst to be used in the reaction include tungstic acid, and sodium tungstate. When the catalyst is used in the reaction, the catalyst is usually used within a range of 0.01 to 0.5 molar rations, as opposed to a mole of the compound (I-a).

In the reaction, the oxidizing agent is usually used within a range of 1 to 1.2 molar ratio(s), as opposed to 1 mole of the compound (I-a).

The reaction temperature of the reaction is usually within a range of −20 to 80° C. The reaction period of the reaction is usually within a range of 0.1 to 12 hours.

When the reaction is completed, water is added to reaction mixtures, and the reaction mixtures are extracted with organic solvent(s), and if necessary, the organic layers are washed with an aqueous solution of a reducing agent (such as sodium sulfite, and sodium thiosulfate) and an aqueous solution of a base (such as sodium hydrogen carbonate). The resulting organic layers are dried and concentrated to obtain the compound (I-b).

Next, a process for preparing the compound (I-c) from the compound (I-b) is described.

The reaction is usually carried out in a solvent. Examples of the solvents to be used in the reaction include halogenated hydrocarbons, nitriles, alcohols, acetic acid, water, and mixed solvents of two or more kinds of the solvents.

Examples of the oxidizing agent to be used in the reaction include mCPBA and peroxide hydrogen.

In the reaction, the oxidizing agent is usually used within a range of 1 to 2 molar ratio(s), as opposed to 1 mole of the compound (I-b).

When peroxide hydrogen is used an oxidizing agent, a base or a catalyst may be added as needed.

Examples of the base to be used in the reaction include sodium carbonate.

When the base is used in the reaction, the base is usually used within a range of 0.01 to 1 molar ration(s), as opposed to 1 mole of the compound (I-b).

Examples of the catalyst to be used in the reaction include sodium tungstate.

When the catalyst is used in the reaction, the base is usually used within a range of 0.01 to 0.5 molar rations, as opposed to 1 mole of the compound (I-b).

The reaction temperature of the reaction is usually within a range of −20 to 120° C. The reaction period of the reaction is usually within a range of 0.1 to 12 hours.

When the reaction is completed, water is added to reaction mixtures, and the reaction mixtures are extracted with organic solvent(s), and if necessary, the organic layers are washed with an aqueous solution of a reducing agent (such as sodium sulfite, and sodium thiosulfate) and an aqueous solution of a base (such as sodium hydrogen carbonate). The resulting organic layers are dried and concentrated to obtain the compound (I-c).

Also, the compound (I-c) can be prepared by reacting the compound (I-a) with an oxidizing agent in one step (one-spot).

The reaction may be carried out by using the oxidizing agent in a ratio of 2 to 5 molar ratios as opposed to 1 mole of the compound (I-a) according to the process for preparing the compound (I-c) from the compound (I-b).

### Process 2

A compound represented by formula (II-b) (hereinafter, referred to as compound (II-b)) or a compound represented by formula (II-c) (hereinafter, referred to as compound (II-c)) can be prepared by reacting a compound represented by formula (II-a)) (hereinafter, referred to as compound (II-a)) with an oxidizing agent.

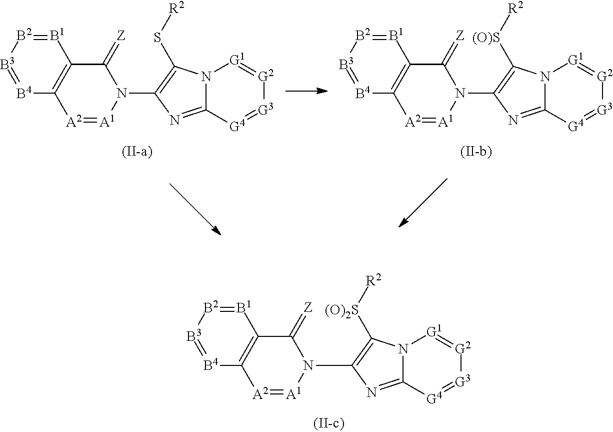

[wherein the symbols are the same as defined above.]

These reactions can be carried out according to the process 1.

### Process 3

A compound represented hv formula (I-S) (hereinafter, referred to as compound (I-S)) can be prepared by reacting a compound represented by formula (I-O) (hereinafter, referred to as compound (I-O)) with a sulfurizing agent.

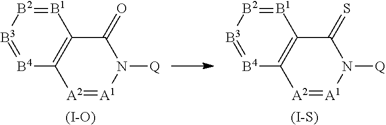

[wherein the symbols are the same as defined above.]

The reaction is carried out in the presence or the absence of a solvent. Examples of the solvent to be used in the reaction include ethers such as tetrahydrofuran (hereinafter, referred to as THF), 1,2-dimethoxyethane (hereinafter, referred to as DME), methyl tert-butyl ether (hereinafter, referred to as MTBE) and diethyl ether (hereinafter, collectively referred to as ethers), halogenated hydrocarbons: aromatic hydrocarbons such as toluene and xylene (hereinafter, referred to as aromatic hydrocarbons); nitriles; nitrogen-containing aromatic compounds such as pyridine, picoline, lutidine, and quinoline (hereinafter, collectively referred to as nitrogen-containing aromatic compounds); and mixed solvents of two or more kinds of the solvents.

Examples of the sulfurizing agent to be used in the reaction include diphosphorus pentasulfide, a Lawesson's reagent (2,4-bis-(4-methoxyphenyl)-1,3-dithia-2,4-diphosphetane 2,4-disulfide), and the like.

In the reaction, the sulfurizing agent is usually used within a range of 1 to 3 molar ratio(s), as opposed to 1 mole of the compound (I-O).

The reaction temperature of the reaction is usually within a range of 0 to 200° C. The reaction period of the reaction is usually within a range of 1 to 24 hours.

When the reaction is completed, water is added to reaction mixtures, and the reaction mixtures are extracted with organic solvent(s), and the organic layers are worked up (for example, drying and concentration) to obtain the compound (I-S).

### Process 4

The compound (I-O) can be prepared by reacting a compound represented by formula (M-1) (hereinafter, referred to compound (M-1)) with a compound represented by formula (M-2) (hereinafter, referred to as compound (M-2)) in the presence of a base.

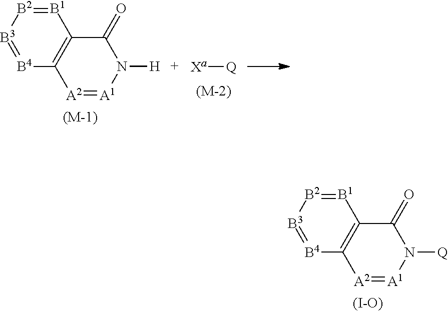

[wherein, X^{a} represents a halogen atom, and the other symbols are the same as defined above.]

The reaction is usually carried out in a solvent. Examples of the solvents to be used in the reaction include ethers; aromatic hydrocarbons; nitriles; polar aprotic solvents such as dimethylformamide (hereinafter, referred to as DMF), N-methyl pyrrolidone (hereinafter, referred to as NMP), and dimethyl sulfoxide (hereinafter, referred to DMSO) (hereinafter, collectively referred to as polar aprotic solvents); and mixed solvents of two or more kinds of the solvents.

Examples of the base include organic bases such as triethylamine, diisopropylethylamine, pyridine, and 4-dimethylaminopyridine (hereinafter, collectively referred to as organic bases); alkali metal carbonates such as sodium carbonate, and potassium carbonate (hereinafter, collectively referred to as alkali metal carbonates); and alkali metal hydrides such as sodium hydride (hereinafter, collectively referred to as alkali metal hydrides).

In the reaction, the compound (M-2) is usually used within a range of 0.8 to 1.2 molar ratio(s), and the base is usually used within a range of 1 to 3 molar ratio(s), as opposed to 1 mole of the compound (M-1).

In the reaction, as needed, metal catalyst may be used. Examples of the metal catalyst include copper catalysts such as copper(I) iodide, copper(I) bromide, copper(I) bromide, copper(I) oxide, copper (I) trifluoromethanesulfonate benzene complex, tetrakis(acetonitrile)copper(I) hexafluorophosphate, and copper(I) 2-thiophenecarboxylate; and nickel catalysts such as bis(cyclooctadiene)nickel(0) and nickel(II) chloride; palladium catalysts such as palladium(II) acetate, tetrakis(triphenylphosphine)palladium(0), and tris(dibenzylideneacetone)dipalladium(II).

When the metal catalyst is used in the reaction, the metal catalyst is usually used within a range of 0.01 to 0.5 molar ratios, as opposed to 1 mole of the compound (M-1).

In the reaction, as needed, a ligand may be used. Examples of the ligand include triphenylphosphine, 4,5-bis(diphenylphosphino)-9,9-dimethylxanthene (hereinafter, referred to as Xantphos), 2,2′-bis(diphenylphoshino)-1,1′-binaphthyl, 1,1′-bis(diphenylphoshino)ferrocene, 2-dicyclohexylphosphino-2′,4′,6′-triisopropylbiphenyl, 2-dicyclohexylphosphino-2′,6′-dimethoxybiphenyl, 1,2-bis(diphenylphosphino)ethane, 2,2′-bipyridine, 2-aminoethanol, 8-hydroxyquinoline, 1,10-phenanthroline, trans-1,2-cyclohexanediamine, trans-N,N′-dimethylhexane-1,2-diamine, N,N′-diethylenediamine and the like.

When the ligand is used in the reaction, the ligand is used within a range of 0.01 to 0.5 molar ratios, as 1 mole of the compound (M-1).

The reaction temperature is usually within a range of −20 to 150° C. The reaction period of the reaction is usually within a range of 0.5 to 24 hours.

When the reaction is completed, water is added to the reaction mixture, and the reaction mixtures are extracted with organic solvent(s), and the organic solvents are worked up (for example, drying and concentration) to obtain the compound (I-O).

The compound (M-1) is a commercially available compound, or can be prepared according to a well-known method.

### Process 5

A compound represented by formula (I-O-a) (hereinafter, referred to as compound (I-O-a)) can be prepared according to the below-mentioned scheme.

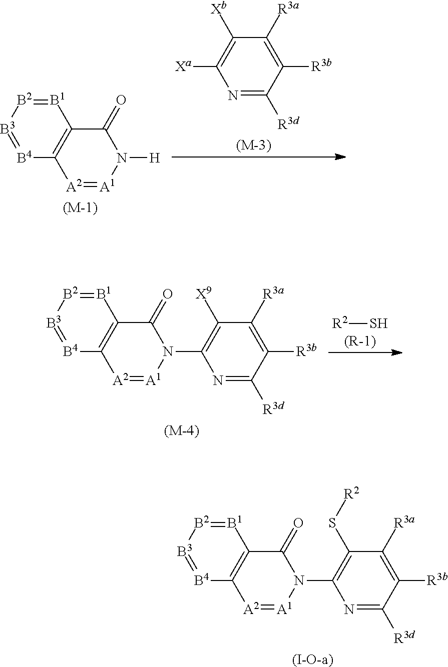

[wherein, X^{b} represents fluorine atom, a chlorine atom, or a bromine atom, and the other symbols are the same as defined above.]

A compound represented by formula (M-4) (hereinafter, referred to as compound (M-4)) can be prepared by using a compound represented by formula (M-3) (hereinafter, referred to as compound (M-3)) in place of the compound (M-2) according to the process 4.

The compound (M-3) is publically known, or can be prepared according to the method described in, for example, WO 2015/187845.

The compound (I-O-a) can be prepared by reacting the compound (M-4) with a compound represented by formula (R-1) (hereinafter, referred to as compound (R-1)) in the presence of a base.

The reaction is usually carried out in a solvent. Examples of the solvents to be used in the reaction include ethers, aromatic hydrocarbons, nitriles, polar aprotic solvents, water, and mixed solvents of two or more kinds of the solvents.

Examples of the bases to be used in the reaction include alkali metal carbonates, alkali metal hydrides, and organic bases.

In the reaction, the compound (R-1) is usually used within a range of 1 to 3 molar ratio(s), and the base is usually used within a range of 1 to 3 molar ratio(s), as opposed to 1 mole of the compound (M-4).

The reaction temperature is usually within a range of −20 to 150° C. The reaction period of the reaction is usually within a range of 0.5 to 24 hours.

When the reaction is completed, water is added to the reaction mixtures, and the reaction mixtures are extracted with organic solvent(s), and the organic solvents are worked up (for example, drying and concentration) to give the compound (I-O-a).

The compound (R-3) is

The compound (R-3) is a commercially available compound, or can be prepared according to a well-known method.

### Process 6

A compound represented by formula (II-O-a) (hereinafter, referred to as compound (II-O-a)) can be prepared according to the below-mentioned scheme.

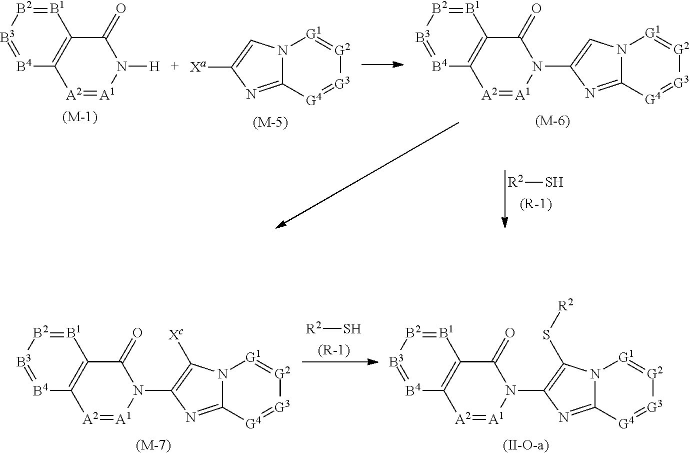

[wherein, X^{c} represents a chlorine atom, a bromine atom or an iodine atom, and the other symbols are the same as above.]

A compound represented by formula (M-6) (hereinafter, referred to as compound (M-6)) can be prepared by using a compound represented by formula (M-5) (hereinafter, referred to as compound (M-5)) in place of the compound (M-2) according to the process 4.

The compound (M-5) is publically known, or can be prepared according to the method described in, for example, WO 2015/157093, WO 2016/109706, Organic & Biomolecular Chemistry, 2017, 15, 4199, ad Europian Journal of Medicinal Chemistry, 2016, 123, 916.

A compound represented by formula (M-7) (hereinafter, referred to as compound (M-7)) can be prepared by reacting the compound (M-6) with a halogenating agent.

The reaction is usually carried out in a solvent. Examples of the solvent include alcohols, nitriles, ethers, aromatic hydrocarbons, ethers, aromatic hydrocarbons, polar aprotic solvents, halogenated hydrocarbons, water, and mixed solvents of two or more kinds of the solvents.

Examples of the halogenating agent include chlorine, bromine, iodine, N-chlorosuccinimide, N-bromosuccinimide, and N-iodosuccinimide.

In the reaction, the halogenating agent is usually used within a range of 1 to 20 molar ratio(s), as opposed to 1 mole of the compound (M-6).

The reaction temperature of the reaction is usually within a range of −20 to 200° C. The reaction period of the reaction is usually within a range of 0.1 to 72 hours.

When the reaction is completed, water is added to the reaction mixtures, and the reaction mixtures are extracted with organic solvent(s), and the organic solvents are worked up (for example, drying and concentration) to give the compound (M-7).

The compound (II-O-a) can be prepared by reacting the compound (M-6), the compound (R-1) and a halogenating agent.

The reaction is usually carried out in a solvent. Examples of the solvent include alcohols, nitriles, ethers, aromatic hydrocarbons, ethers, aromatic hydrocarbons, polar aprotic solvents, halogenated hydrocarbons, water, and mixed solvents of two or more kinds of the solvents.

Examples of the halogenating agent include chlorine, bromine, iodine, N-chlorosuccinimide, N-bromosuccinimide, and N-iodosuccinimide.

In the reaction, the compound (R-1) is usually used within a range of 1 to 20 molar ratio(s), and the halogenating agent is usually used within a range of 1 to 20 molar ratio(s), as opposed to 1 mole of the compound (M-6). The reaction temperature of the reaction is usually within a range of −20 to 200° C. The reaction period of the reaction is usually within a range of 0.1 to 72 hours.

When the reaction is completed, water is added to the reaction mixtures, and the reaction mixtures are extracted with organic solvent(s), and the organic solvents are worked up (for example, drying and concentration) to give the compound (II-O-a).

The compound (II-O-a) can be also prepared by reacting the compound (M-7) with the compound (R-1) in the presence of a metal catalyst and a base.

The reaction is usually carried out in a solvent. Examples of the solvents to be used in the reaction include alcohols, nitriles, ethers, aromatic hydrocarbons, polar aprotic solvents, water, and mixed solvents of two or more kinds of the solvents.

Examples of the meal catalyst to be used in the reaction include palladium catalysts such as tetrakis(triphenylphosphine)palladium(0), 1,1′-bis(diphenylphosphino)ferrocene palladium(II) dichloride, tris(dibenzylideneacetone)dipalladium(0), and palladium(II) acetate; nickel catalysts such as bis(cyclooctadiene)nickel(0) and nickel(II) chloride; and copper catalyst such as copper(I) iodide and copper(I) chloride.

Examples of the base to be used in the reaction include alkali metal hydrides, alkali metal carbonates, and organic bases.

A ligand may be used in the reaction. Examples of the ligand to be used in the reaction include triphenylphosphine, Xantphos, 2,2′-bis(diphenylphoshino)-1,1′-binaphthyl, 1,1′-bis(diphenylphoshino)ferrocene, 2-dicyclohexylphosphino-2′,4′,6′-triisopropylbiphenyl, 2-dicyclohexylphosphino-2′,6′-dimethoxybiphenyl, 1,2-bis(diphenylphosphino)ethane, 2,2′-bipyridine, 2-aminoethanol, 8-hydroxyquinoline, and 1,10-phenanthroline.

In the reaction, the compound (R⁵) is usually used within a range of 1 to 20 molar ratio(s), the metal catalyst is usually used within a range of 0.01 to 0.5 molar ratios, the ligand is usually used within a range of 0.01 to 1 molar ratio(s), and the base is usually used within a range of 0.1 to 5 molar ratio(s), as opposed to 1 mole of the compound (M-6).

When a ligand is used in the reaction, the ligand is usually used within a range of 0.01 to 1 molar ratio(s) as opposed to 1 mole of the compound (M-7).

In the reaction, the compound (R-1) is usually used within a range of 1 to 20 molar ratio(s), the metal catalyst is usually used within a range of 0.01 to 0.5 molar ratios, and the base is usually used within a range of 0.1 to 5 molar ratio(s), as opposed to 1 mole of the compound (M-7).

The reaction temperature of the reaction is usually within a range of −20 to 200° C. The reaction period of the reaction is usually within a range of 0.1 to 72 hours.

When the reaction is completed, water is added to the reaction mixtures, and the reaction mixtures are extracted with organic solvent(s), and the organic solvents are worked up (for example, drying and concentration) to give the compound (II-O-a).

### Process 7

A compound represented by formula (I-1) (hereinafter, referred to as compound (I-1)) can be prepared according to the below-mentioned scheme.

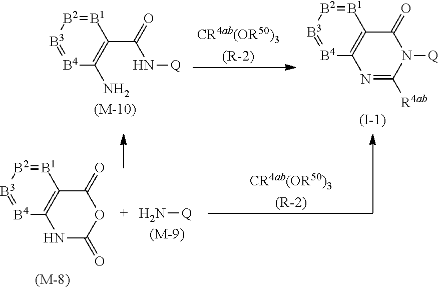

[wherein, R⁵⁰ represents a methyl group or an ethyl group, R^{4ab} represents a C1-C6 chain hydrocarbon group optionally having one or more halogen atoms, a C3-C7 cycloalkyl group optionally having one or more halogen atoms, or a hydrogen atom, and the other symbols are the same as defined above.]

A compound represented by formula (M-10) (hereinafter, referred to as compound (M-10)) can be prepared by reacting a compound represented by formula (M-8) (hereinafter, referred to as compound (M-8)) with a compound represented by formula (M-9) (hereinafter, referred to as compound (M-9)).

The reaction is usually carried out in a solvent. Examples of the solvents to be used in the reaction include alcohols, ethers, aromatic hydrocarbons, nitriles, polar aprotic solvents, and mixed solvents of two or more kinds of the solvents.

A base may be used in the reaction as needed. Examples of the base include organic bases. When the base is used in the reaction, the base is usually used within a range of 1 to 5 molar ratio(s), as opposed to 1 mole of the compound (M-8).

In the reaction, the compound (M-9) is usually used within a range of 1 to 5 molar ratio(s), as opposed to 1 mole of the compound (M-8).

The reaction temperature of the reaction is usually within a range of 0 to 150° C. The reaction period of the reaction is usually within a range of 0.5 to 12 hours. When the reaction is completed, water is added to the reaction mixtures, and the reaction mixtures are extracted with organic solvent(s), and the organic solvents are worked up (for example, drying and concentration) to give the compound (M-10).

The compound (M-8) can be prepared according to the method described in, for example, Journal of Biological Chemistry, 2016, 291, 14146.

The compound (I-1) can be prepared by reacting the compound (M-10) with a compound represented by formula (R-2) (hereinafter, referred to as compound (R-2)).

The reaction is carried out in the presence or the absence of a solvent. Examples of the solvents to be used in the reaction include ethers, aromatic hydrocarbons, halogenated hydrocarbons, nitriles, polar aprotic solvents and mixed solvents of two or more kinds of the solvents.

In the reaction, the compound (R-2) is usually used within a range of 1 to 50 molar ratio(s), as opposed to 1 mole of the compound (M-10).

An acid or a base may be used in the reaction as needed.

Examples of the acid to be used in the reaction include sulfonic acids such as para-toluenesulfonic acid; carbonic acids such as acetic acid; and polyphosphoric acids.

Examples of the base to be used in the reaction include organic bases.

When the acid or the base is used in the reaction, the acid is usually used within a range of 0.01 to 5 molar ratio(s), and the base is usually used within a range of 1 to 5 molar ratio(s), as opposed to 1 mole of the compound (M-10).

The reaction temperature of the reaction is usually within a range of 0 to 150° C. The reaction period of the reaction is usually within a range of 0.5 to 24 hours.

When the reaction is completed, water is added to reaction mixtures, and the reaction mixtures are extracted with organic solvent(s), and the organic layers are worked up (for example, drying and concentration) to obtain the compound (I-1).

The compound (M-1) is a commercially available compound, or can be prepared according to a well-known method.

Also, the compound (I-1) can be prepared by reacting the compound (M-8), the compound (M-9) and the compound (R-2) in one step (one-spot).

In the reaction, the compound (M-9) is usually used within a range of 1 to 5 molar ratio(s), and the compound (R-2) is usually used within a range of 1 to 50 molar ratio(s), as opposed to 1 mole of the compound (M-8), according to the method for preparing the compound (I-1) from the compound (M-10).

### Process 8

A compound represented by formula (I-2) (hereinafter, referred to as compound (I-2) can be prepared by reacting a compound represented by formula (M-11) (hereinafter, referred to as compound (M-11)) with a compound represented by formula (R-3) (hereinafter, referred to as compound (R-3)) in the presence of a base.

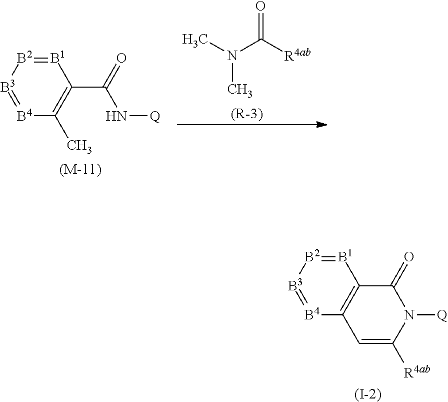

[wherein the symbols are the same as defined above.]

The reaction is usually carried out in a solvent. Examples of the solvents to be used in the reaction include ethers, aromatic hydrocarbons, and mixed solvents of two or more kinds of the solvents.

Examples of the base to be used in the reaction include butyl lithium, s-butyl lithium, t-butyl lithium, lithium diisopropylamide, lithium bis(trimetyhylsilyl)amide, sodium bis(trimethylsilyl)amide; and potassium bis(trimethylsilyl)amide.

In the reaction, the compound (R-3) is usually used within a range of 1 to 15 molar ratio(s), and the base is usually used within a range of 1 to 5 molar ratio(s), as opposed to 1 mole of the compound (M-11).

The reaction temperature is usually within a range of −78 to 25° C. The reaction period of the reaction is usually within a range of 0.5 to 12 hours.

When the reaction is completed, water is added to reaction mixtures, and the reaction mixtures are extracted with organic solvent(s), and the organic layers are worked up (for example, drying and concentration) to obtain the compound (I-2).

The compound (R-3) is a commercially available compound, or can be prepared according to a well-known method.

### Process 9

A compound represented by formula (I-3) (hereinafter, referred to as compound (I-3)) can be prepared according to the below-mentioned scheme.

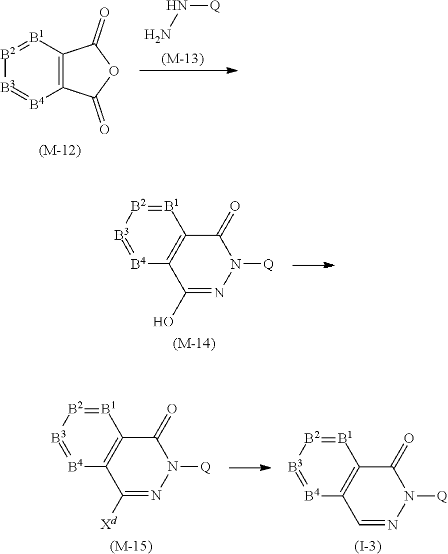

[wherein X^{d} represents a chlorine atom or a bromine atom, and the other symbols are the same as defined above.]

A compound represented by formula (M-14) (hereinafter, referred to as compound (M-14)) can be prepared by reacting a compound represented by formula (M-12) (hereinafter, referred to as compound (M-12)) with a compound represented by formula (M-13) (hereinafter, referred to as compound (M-13)). The reaction can be carried out according to the method described in, for example, Journal of Medicinal Chemistry, 2011, 54, 2102.

A compound represented by formula (M-15) (hereinafter, referred to as compound (M-15)) can be prepared by reacting the compound (M-14) with a halogenating agent.

The reaction is carried out in the presence or the absence of a solvent. Examples of the solvents to be used in the reaction include ethers, aromatic hydrocarbons, halogenated hydrocarbons, and mixed solvents of two or more kinds of the solvents.

Examples of the halogenating agent to be used in the reaction include thionyl chloride, oxalyl chloride, phosphorus oxychloride, phosphorus trichloride, phosphorus pentachloride, thionyl bromide, phosphorus oxybromide, phosphorus tribromide, and phosphorus pentachloride.

In the reaction, the halogenating agent is usually used within a range of 1 to 30 molar ratio(s), as opposed to 1 mole of the compound (M-14).

The reaction temperature is usually within a range of 0 to 100° C. The reaction period of the reaction is usually within a range of 0.1 to 24 hours.

When the reaction is completed, water is added to reaction mixtures, and the reaction mixtures are extracted with organic solvent(s), and the organic layers are worked up (for example, drying and concentration) to obtain the compound (M-15).

The compound (I-3) can be prepared by reacting the compound (M-15) with a hydrogen in the presence of a catalyst. The reaction is usually carried out in a solvent under 1 to 100 bar of hydrogen atmosphere. Examples of the solvent to be used in the reaction include ethers; esters (such as ethyl acetate, and butyl acetate) (hereinafter, collectively referred to as esters); alcohols; water; and mixed solvents comprising two or more thereof.

Examples of the catalysts to be used in the reaction include palladium carbon.

In the reaction, the catalyst is usually used within a range of 0.001 to 0.5 molar ratios, as opposed to 1 mole of the compound (M-15).

An acid or a base may be used in the reaction as needed.

Examples of the acid to be used in the reaction include acetic acid, and hydrochloric acid, and examples of the base to be used in the reaction include tertiary amines such as triethylamine. When the acid is used in the reaction, the acid is usually used within a range of 0.1 to 5 molar ratio(s), as opposed to 1 mole of the compound (M-15). When the base is used in the reaction, the base is usually used within a range of 0.1 to 5 molar ratio(s), as opposed to 1 mole of the compound (M-15).

The reaction temperature is usually within a range of −20 to 100° C. The reaction period of the reaction is usually within a range of 0.1 to 24 hours.

When the reaction is completed, the reaction mixtures are filtered, and as needed, are extracted with organic solvent(s), and the resulting organic layers are worked up (for example, drying and concentration) to obtain the Compound (I-3).

### Process 10

An oxide of the compound represented by formula (I) can be prepared by reacting the compound represented by formula (I) with an oxidizing agent. The reaction can be carried out according to the method described in, for example, the process 1, U.S. patent publication No. 2018/0009778 or WO 2016/121970.

### Process 11

A compound represented by formula (I-4) (hereinafter, referred to as compound (I-4)) can be prepared by reacting the compound (M-10) with sodium sulfite in the presence of a halogenating agent.

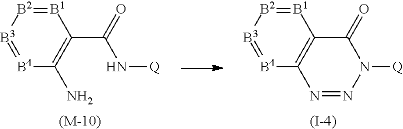

The reaction is carried out in the presence or the absence of a solvent. Examples of the solvents to be used in the reaction include nitriles, polar aprotic solvents, and mixed solvents of two or more kinds of the solvents.

Examples of the halogenating agent to be used in the reaction include chlorine, bromine, iodine, N-chlorosuccinimide, N-bromosuccinimide, and N-iodosuccinimide.

In the reaction, the sodium sulfite is usually used within a range of 1 to 10 molar ratios, and the halogenating agent is usually used within a range of 0.1 to 5 molar ratio(s), as opposed to 1 mole of the compound (M-10).

The reaction temperature is usually within a range of 0 to 100° C. The reaction period of the reaction is usually within a range of 0.5 to 12 hours.

When the reaction is completed, water is added to reaction mixtures, and the reaction mixtures are extracted with organic solvent(s), and the organic layers are worked up (for example, drying and concentration) to obtain the compound (I-4).

Next, a process for preparing preparation intermediate compound is described.

### Reference Process 1

A compound represented by formula (M-2A-b) and a compound represented by formula (M-2A-c) can be prepared by reacting the compound represented by formula (M-2A-a) (hereinafter, referred to as compound (M-2A-a)) with an oxidizing agent.

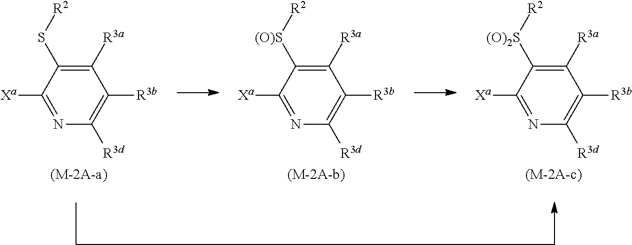

[wherein the symbols are the same as defined above.]

These reactions can be carried out according to the process 1.

### Reference Process 2

The compound (M-2A-a) can be prepared by reacting a compound represented by formula (M-2A-1) (hereinafter, referred to as compound (M-2A-1)) with the compound (R-1) in the presence of a base.

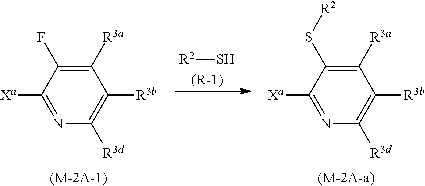

[wherein the symbols are the same as defined above.]

The reaction can be carried out according to the method described in the process 5 for preparing the compound (I-O-a) from the compound (M-4).

The compound (M-2A-1) is a commercially available compound, or can be prepared according to a well-known method.

### Reference Process 3

A compound represented by formula (M-2B-b) and a compound represented by formula (M-2B-c) can be prepared by reacting a compound represented by formula (M-2B-a) with an oxidizing agent.

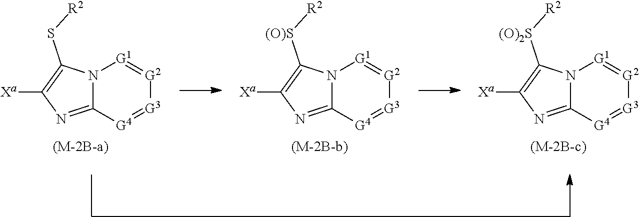

[wherein the symbols are the same as defined above.]

These reactions can be carried out according to the process 1.

### Reference Process 4

A compound represented by formula (M-2B-d) can be prepared according to the below-mentioned scheme.

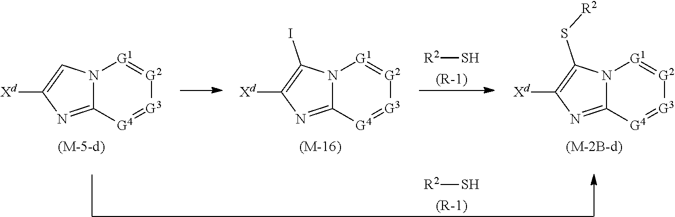

[wherein the symbols are the same as defined above.]

A compound represented by formula (M-16) (hereinafter, referred to as compound (M-16)) can be prepared by reacting a compound represented by formula (M-5-d) (hereinafter, referred to as compound (M-5-d)) with N-iodosuccinimide. The reaction can be carried out according to the method described in the process 6 for preparing the compound (M-7) from the compound (M-6).

The compound (M-2B-d) can be prepared by reacting the compound (M-16) or the compound (M-5-d) with the compound (R-1). These reactions can be carried out according to the method described in the process 6 for preparing the compound (II-O-a) from the compound (M-6) or the compound (M-7).

### Reference Process 5

A compound represented by formula (M-2B-f) can be prepared by reacting a compound represented by formula (M-2B-e) (hereinafter, referred to as compound (M-2B-e)) with silver fluoride in the presence of a metal catalyst.

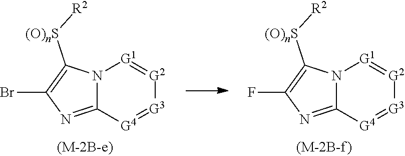

[wherein the symbols are the same as defined above.]

The reaction can be carried out according to the method described in, for example, Journal of the American Chemical Society, 2014, 136, 3792.

### Reference Process 6

A compound represented by formula (M-2B-g) can be prepared by reacting the compound (M-2B-e) with sodium iodide in the presence of a metal catalyst.

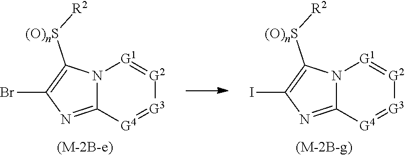

[wherein the symbols are the same as defined above.]

The reaction can be carried out according to the method described in, for example, Journal of the American Chemical Society, 2002, 124, 14844.

### Reference Process 7

A compound represented by formula (M-4A) (hereinafter, referred to as compound (M-4A)) can be prepared by reacting a compound represented by formula (M-17) (hereinafter, referred to as compound (M-17)) with the compound (R-3) in the presence of a base.

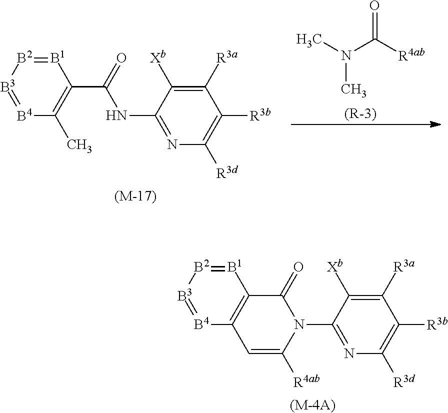

[wherein the symbols are the same as defined above]

The reaction can be carried out by using the compound (M-17) in place of the compound (M-11) according to the process 8.

### Reference Process 8

The compound (M-4B) can be prepared by reacting a compound represented by formula (M-18) (hereinafter, referred to as compound (M-18)) with the compound (R-3) in the presence of a base.

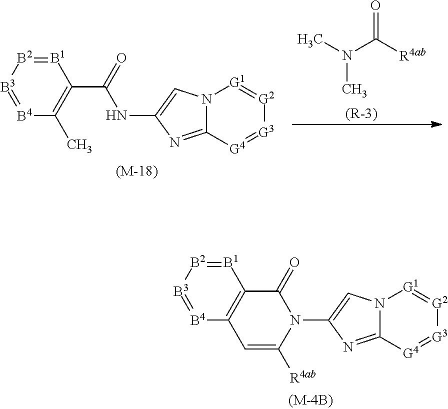

[wherein the symbols are the same as defined above.]

The reaction can be carried out by using the compound (M-18) in place of the compound (M-11) according to the process 8.

### Reference Process 9

The compound (M-11) can be prepared by reacting a compound represented by formula (M-19) (hereinafter, referred to as compound (M-19)) with the compound (M-9) in the presence of a condensation agent.

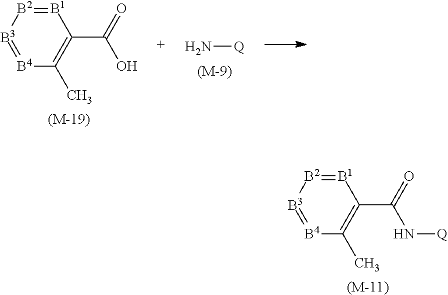

[wherein the symbols are the same as defined above.]

The reaction is usually carried out in a solvent. Examples of the solvents to be used in the reaction include ethers, halogenated hydrocarbons, aromatic hydrocarbons, esters, polar aprotic solvents, nitrogen-containing aromatic hydrocarbons, and mixed solvents of two or more kinds of the solvents.

Examples of the condensation agent include 1-ethyl-3-83-dimetylaminopropyl)carbodiimide (hereinafter, referred to as WSC) and 1,3-dicyclohexylarbodiimide

In the reaction, the compound (M-9) is usually used within a range of 0.8 to 1.2 molar ratio(s), and the condensation agent is usually used within a range of 1 to 2 molar ratio(s), as opposed to 1 mole of the compound (M-19).

A catalyst may be added in the reaction as needed. Examples of the catalyst include 1-hydroxybenzotriazole (hereinafter, referred to as HOBt). When the catalyst is used in the reaction, the catalyst is usually used within a range of 0.01 to 1 molar ratio(s), as opposed to 1 mole of the compound (M-19).

The reaction temperature of the reaction is usually within a range of 0 to 200° C. The reaction period of the reaction is usually within a range of 0.1 to 24 hours.

When the reaction is completed, water is added to reaction mixtures, and the reaction mixtures are extracted with organic solvent(s), and the organic layers are worked up (for example, drying and concentration) to obtain the compound (M-11).

The compound (M-19) is a commercially available compound, or can be prepared according to a well-known method.

### Reference Process 10

The compound (M-17) can be prepared by reacting the compound (M-19) with a compound represented by formula (M-20) (hereinafter, referred to as compound (M-20)) in the presence of a condensation agent.

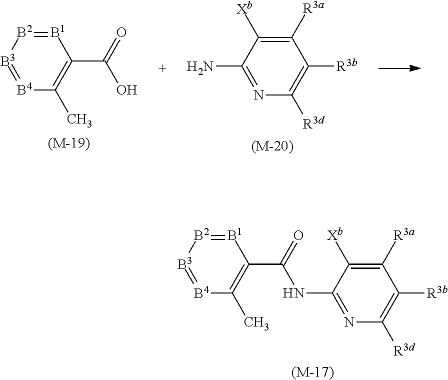

[wherein the symbols are the same as defined above.]

The reaction can be carried out by using the compound (M-20) in place of the compound (M-9) according to the Reference Process 9.

Reference Process 11 The compound (M-18) can be prepared by reacting the compound (M-19) with a compound represented by formula (M-21) (hereinafter, referred to as compound (M-21)) in the presence of a condensation agent.

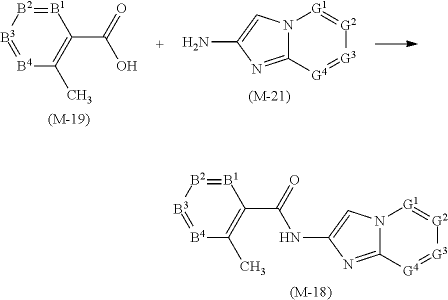

[wherein the symbols are the same as defined above.]

The reaction can be carried out by using the compound (M-21) in place of the compound (M-9) according to the Reference Process 9.

### Reference Process 11

A compound represented by formula (M-4C) can be prepared according to the below-mentioned scheme.

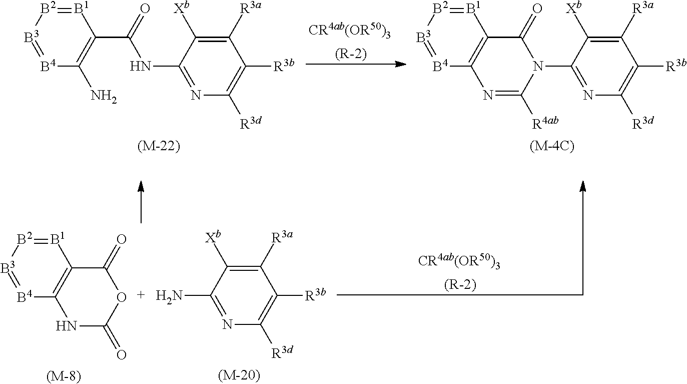

[wherein the symbols are the same as defined above.]

These reactions can be carried out according to the Process 7.

### Reference Process 12

A compound represented by formula (M-4D) can be prepared according to the below-mentioned scheme.

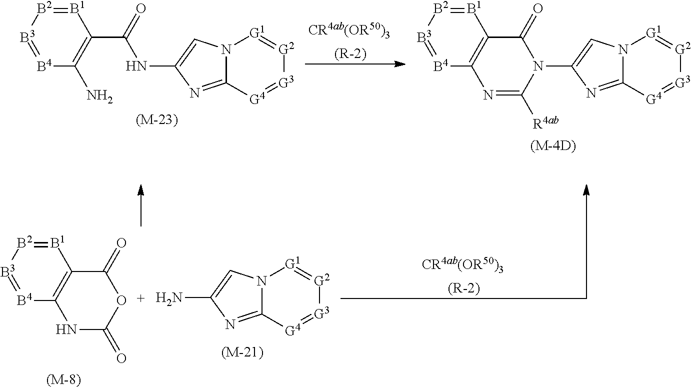

[wherein the symbols are the same as defined above.]

These reactions can be carried out according to the Process 7.

### Reference Process 13

The compound (M-12) can be prepared by a dehydration condensation of a compound represented by formula (M-24) (hereinafter, referred to as compound (M-24)).

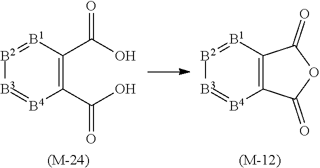

[wherein the symbols are the same as defined above.] The reaction can be carried out according to the method described in, for example, Organic Letters, 2010, 12, 4796.

The compound (M-24) is a commercially available compound, or can be prepared according to a well-known method.

### Reference Process 14

A compound represented by formula (M-4E) can be prepared according to the below-mentioned scheme.

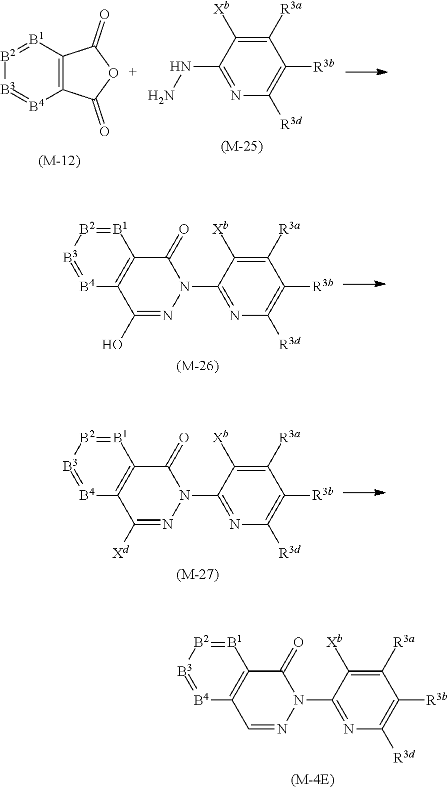

[wherein the symbols are the same as defined above.]

These reactions can be carried out according to the Process 9.

A compound represented by formula (M-4F) can be prepared according to the below-mentioned scheme.

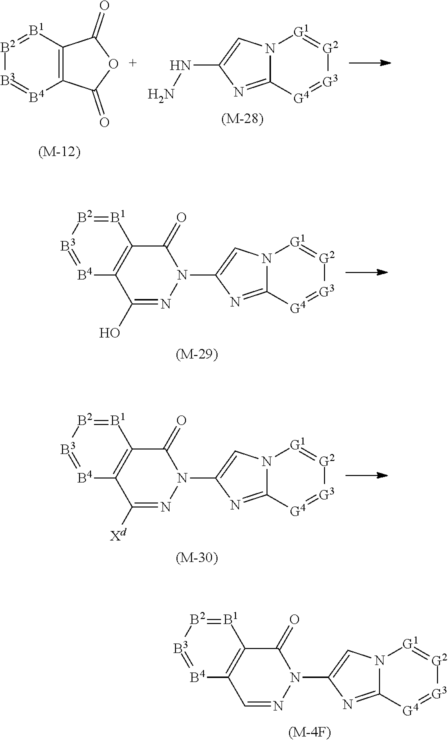

[wherein the symbols are the same as defined above.]

These reactions can be carried out according to the Process 9.

### Reference Process 16

The compound (M-9) can be prepared by reacting the compound (M-2) with ammonia.

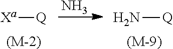

[wherein the symbols are the same as defined above.]

The reaction can be carried out according to the method described in, for example, Journal of Medicinal Chemistry, 1980, 23, 1376 or WO 2010/130665.

The compound (M-9) can be prepared by according to the method described in WO 2018/052136.

### Reference Process 17

The compound (M-20) can be prepared by reacting the compound (M-3) with ammonia.

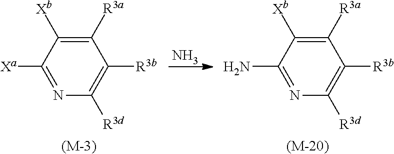

[wherein the symbols are the same as defined above.]

The reaction can be carried out according to the Reference Process 16.

### Reference Process 18

The compound (M-21) can be prepared by reacting the compound (M-5) with ammonia.

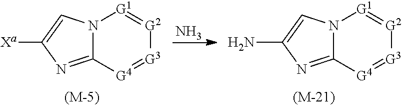

[wherein the symbols are the same as defined above.]

The reaction can be carried out according to the Reference Process 16.

### Reference Process 19

The compound (M-13) can be prepared by reacting the compound (M-2) with hydrazine monohydrate.

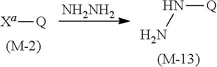

[wherein the symbols are the same as defined above.]

The reaction can be carried out according to the method described in, for example WO 2018/008727.

### Reference Process 20

The compound (M-25) can be prepared by reacting the compound (M-3) with hydrazine monohydrate.

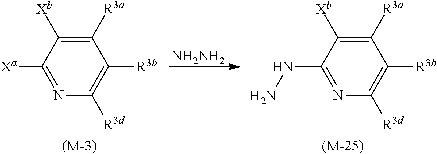

[wherein the symbols are the same as defined above.]

The reaction can be carried out according to the Reference Process 19.

### Reference Process 21

The compound (M-28) can be prepared by reacting the compound (M-5) with hydrazine monohydrate.

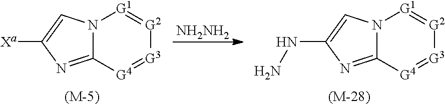

[wherein the symbols are the same as defined above.]

The reaction can be carried out according to the Reference Process 19.

### Reference Process 22

A compound represented by formula (M-1A) can be prepared by reacting a compound represented by formula (M-31) (hereinafter, referred to as compound (M-31)) with a compound represented by formula (M-32) (hereinafter, referred to as compound (M-32)).

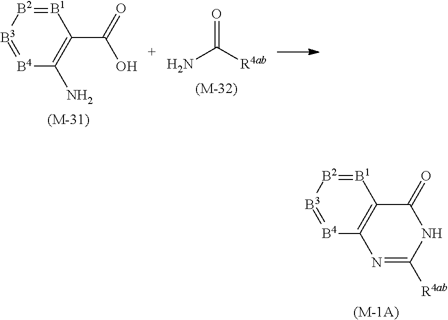

[wherein the symbols are the same as defined above.]

The reaction can be carried out according to the method described in, for example, European Journal of Medicinal Chemistry, 2012, 50, 264.

The compound (M-31) and the compound (M-32) are commercially available compounds, or can be prepared according to a well-known method.

### Reference Process 23

A compound represented by formula (M-1B) can be prepared according to the below-mentioned scheme.

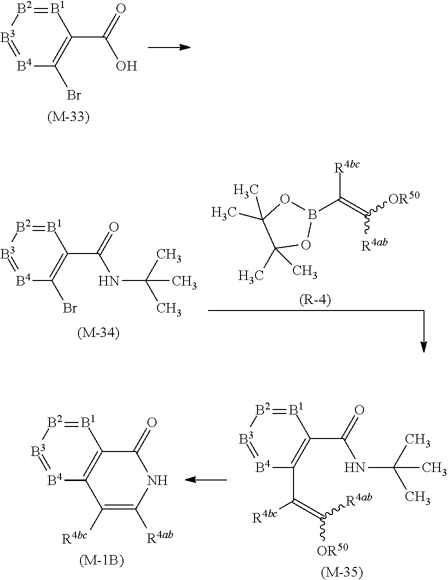

[wherein R^{4bc} represents a C1-C6 chain hydrocarbon group optionally having one or more halogen atoms, a C3-C7 cycloalkyl group optionally having one or more halogen atoms, or a hydrogen atom, and the other symbols are the same as defined above.

The reaction can be carried out according to the method described in, for example, European Journal of Organic Chemistry, 2016, 4171.

A compound represented by formula (M-33) and a compound represented by formula (M-34) are commercially available compounds, or can be prepared according to a well-known method.

### Reference Process 24

A compound represented by formula (M-1C) can be prepared by reacting a compound represented by formula (M-36) (hereinafter, referred to as compound (M-36)) with hydrazine monohydrate.

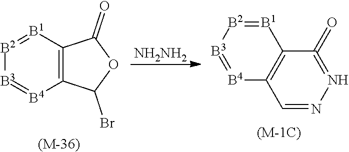

[wherein the symbols are the same as defined above.]

The reaction can be carried out according to the method described in, for example, WO 2011/159854.

The compound (M-36) can be prepared according to the method described in, for example, Bioorganic & Medicinal Chemistry, 2013, 23, 1063.

The present compound or the present compound X may be mixed or combined with one or more kinds of ingredients selected from a group consisting of the following Group (a), Group (b), Group (c), and Group (d) (hereinafter, referred to as Present ingredient).

The above-mentioned mixing or combining represents a use of the Present compound or the present compound X and the Present ingredient at same time, separately or at certain intervals.

When the Present compound or the present compound X and the present ingredient are used at the same time, the Present compound or the present compound X and the Present ingredient may be contained in separate formulations respectively or may be contained in the same one formulation.

One aspect of the present invention is a composition comprising one or more ingredients selected from Group (a) or Group (b) as well as the Present compound.

One aspect of the present invention is a composition comprising one or more ingredients selected from a group consisting of the following Group (a), Group (b), Group (c), and Group (d) as well as the Present compound X (hereinafter, referred to as Composition A).

Group (a) is a group consisting of each active ingredient as Acetylcholinesterase inhibitors (for example, carbamate insecticides, or organophosphorus insecticides), GABA-gated chloride channel blockers (for example, phenylpyrazol insecticides), Sodium channel modulators (for example, pyrethroid insecticides), Nicotinic acetylcholine receptor competitive modulators (for example, neonicotinoid insecticides), Nicotinic acetylcholine receptor allosteric modulators, Glutamatergic chlorine ion channel allosteric modulators (for example, macrolide insecticides), Juvenile hormone mimic, Multisite inhibitors, chordotonal organ TRPV channel modulators, Mites growth inhibitors, Mitochondria ATP biosynthetic enzyme inhibitors, Uncouplers of oxidative phosphorylation, Nicotinic acetylcholine receptor channel blocker (for example, Nereistoxin insecticides), Chitin synthesis inhibitors, Molting inhibitors, Ecdysone receptor agonist, Octopamine receptor agonist, Inhibitors of Mitochondrial electron transport system complex I, II, III and IV, Voltage-dependent sodium channel blockers, Acetyl CoA carboxylase inhibitor, Ryanodine receptor modulator (for example, Diamide insecticides), Chordotonal organ modulators, Microbial pesticides; and
the other insecticidal, miticidal or nematicidal active ingredients.

These ingredients are classified as a class based on the action mechanism of IRAC.

Group (b) is a group consisting of Nucleic acid synthesis inhibitors (for example, Phenylamide fungicides, or Acylamino acid fungicides), cell division and cytoskeleton inhibitors (for example, MBC fungicides), Respiratory inhibitors (for example, QoI fungicides or Qil fungicides), Amino acid synthesis and protein synthesis inhibitors (for example, anilinopyridine fungicides), Signal transduction inhibitors, Lipid synthesis and membrane synthesis inhibitors, sterol biosynthesis inhibitors (for example, DMI fungicides such as triazole), cell wall synthesis inhibitors, Melanin synthesis inhibitors, Plant defense inducers, Other action point contact active fungicides, Microbial fungicides, and the other fungicidal ingredients. These are classified as a class based on the action mechanism of FRAC.

Group (c) is a plant growth modulating ingredient group (including Mycorrhizal fungi, and Root nodule bacteria).

Group (d) is a repellent ingredient group.

Examples of the combination of the Present ingredient and the Present compound X are described below. For example, alanycarb+SX represents a combination of alanycarb and SX.

The symbol of “SX” represents any one of the Present compound X selected from the Compound Class SX₁ to the Compound Class SX₂₈₃₂. Also, all of the below-mentioned present active ingredient are known ingredients, and are commercially available or may be produced by the known method. If the present ingredient is a bacterium, it is available from the bacterial authority depository. The numerical number in bracket represents a CAS RN(Register Trademark).

Combination of the Present ingredient of the above Group (a) and the Present compound:
abamectin+SX, acephate+SX, acequinocyl+SX, acetamiprid+SX, acetoprole+SX, acrinathrin+SX, acynonapyr+SX, afidopyropen+SX, afoxolaner+SX, alanycarb+SX, aldicarb+SX, allethrin+SX, alpha-cypermethrin+SX, alpha-endosulfan+SX, aluminium phosphide+SX, amitraz+SX, azadirachtin+SX, azamethiphos+SX, azinphos-ethyl+SX, ainphos-methyl+SX, axocyclotin+SX, bark of Celastrus angulatus+SX, bendiocarb+SX, benfluthrin+SX, benfuracarb+SX, bensultap+SX, benzoximate+SX, benzpyrimoxan+SX, beta-cyfluthrin+SX, beta-cypermethrin+SX, bifenazate+SX, bifenthrin+SX, bioallethrin+SX, bioresmethrin+SX, bistrifluron+SX, borax+SX, boric acid+SX, broflanilide+SX, bromopropylate+SX, buprofezin+SX, butocarboxim+SX, butoxycarboxim+SX, cadusafos+SX, calcium phosphide+SX, carbaryl+SX, carbofuran+SX, carbosulfan+SX, cartap hydrochloride+SX, cartap+SX, chinomethionat+SX, chlorantraniliprole+SX, chlordane+SX, chlorethoxyfos+SX, chlorfenapyr+SX, chlorfenvinphos+SX, chlorfluazuron+SX, chlormephos+SX, chloropicrin+SX, chlorpyrifos+SX, chlorpyrifos-methyl+SX, chromafenozide+SX, clofentezine+SX, clothianidin+SX, concanamycin A+SX, coumaphos+SX, cryolite+SX, cyanophos+SX, cyantraniliprole+SX, cycloniliprole+SX, cycloprothrin+SX, cycloxaprid+SX, cyenopyrafen+SX, cyflumetofen+SX, cyfluthrin+SX, cyhalodiamide+SX, cyhalothrin+SX, cyhexatin+SX, cypermethrin+SX, cyphenothrin+SX, cyromazine+SX, dazomet+SX, deltamethrin+SX, demeton-S-methyl+SX, diafenthiuron+SX, diazinon+SX, dichlorvos+SX, dicloromezotiaz+SX, dicofol+SX, dicrotophos+SX, diflovidazin+SX, diflubenzuron+SX, dimefluthrin+SX, dimethoate+SX, dimethylvinphos+SX, dimpropyridaz+SX, dinotefuran+SX, disodium octaborate+SX, disulfoton+SX, DNOC(2-methyl-4,6-dinitrophenol)+SX, doramectin+SX, dried leaves of Dryopteris filix-mas+SX, emamectin-benzoate+SX, empenthrin+SX, endosulfan+SX, EPN(O-ethyl 0-(4-nitrophenyl)phenylphosphonothioate)+SX, epsilon-metofluthrin+SX, epsilon-momfluorothrin+SX, esfenvalerate+SX, ethiofencarb+SX, ethion+SX, ethiprole+SX, ethoprophos+SX, etofenprox+SX, etoxazole+SX, extract of Artemisia absinthium+SX, extract of Cassia nigricans+SX, extract of Clitoria ternatea+SX, extract of Symphytum officinale+SX, extracts or simulated blend of Chenopodium ambrosioides+SX, extract of Tanacetum vulgare+SX, extract of Urtica dioica+SX, extract of Viscum album+SX, famphur+SX, fenamiphos+SX, fenazaquin+SX, fenbutatin oxide+SX, fenitrothion+SX, fenobucarb+SX, fenoxycarb+SX, fenpropathrin+SX, fenpyroximate+SX, fenthion+SX, fenvalerate+SX, fipronil+SX, flometoquin+SX, flonicamid+SX, fluacrypyrim+SX, fluazaindolizine+SX, fluazuron+SX, flubendiamide+SX, flucycloxuron+SX, flucythrinate+SX, fluensulfone+SX, flufenoprox+SX, flufenoxuron+SX, flufiprole+SX, flumethrin+SX, flupyradifurone+SX, flupyrimin+SX, fluralaner+SX, fluvalinate+SX, fluxametamide+SX, formetanate+SX, fosthiazate+SX, furamethrin+SX, furathiocarb+SX, gamma-cyhalothrin+SX, GS-omega/kappa HXTX-Hvla peptide+SX, halfenprox+SX, halofenozide+SX, heptafluthrin+SX, heptenophos+SX, hexaflumuron+SX, hexythiazox+SX, potassium salt of hop beta acid+SX, hydramethylnon+SX, hydroprene+SX, imicyafos+SX, imidacloprid+SX, imidaclothiz+SX, imiprothrin+SX, indoxacarb+SX, isocycloseram+SX, isofenphos+SX, isoprocarb+SX, isopropyl-O-(methoxyaminothiophosphoryl) salicylate+SX, isoxathion+SX, ivermectin+SX, kadethrin+SX, kappa-tefluthrin+SX, kappa-bifenthrin+SX, kinoprene+SX, lambda-cyhalothrin+SX, lenoremycin+SX, lepimectin+SX, lime sulfur+SX, lotilaner+SX, lufenuron+SX, machine oil+SX, malathion+SX, mecarbam+SX, meperfluthrin+SX, metaflumizone+SX, metam+SX, methamidophos+SX, methidathion+SX, methiocarb+SX, methomyl+SX, methoprene+SX, methoxychlor+SX, methoxyfenozide+SX, methyl bromide+SX, metofluthrin+SX, metolcarb+SX, metoxadiazone+SX, mevinphos+SX, milbemectin+SX, milbemycin oxime+SX, momfluorothrin+SX, monocrotophos+SX, moxidectin+SX, naled+SX, neem oil+SX, nicotine+SX, nicotine-sulfate+SX, nitenpyram+SX, novaluron+SX, noviflumuron+SX, oil of the seeds of Chenopodium anthelminticum+SX, omethoate+SX, oxamyl+SX, oxazosulfyl+SX, oxydemeton-methyl+SX, parathion+SX, parathion-methyl+SX, permethrin+SX, phenothrin+SX, phenthoate+SX, phorate+SX, phosalone+SX, phosmet+SX, phosphamidon+SX, phosphine+SX, phoxim+SX, pirimicarb+SX, pirimiphos-methyl+SX, prallethrin+SX, profenofos+SX, profluthrin+SX, propargite+SX, propetamphos+SX, propoxur+SX, propylene glycol alginate+SX, prothiofos+SX, pyflubumide+SX, pymetrozine+SX, pyraclofos+SX, pyrethrins+SX, pyridaben+SX, pyridalyl+SX, pyridaphenthion+SX, pyrifluquinazone+SX, pyrimidifen+SX, pyriminostrobin+SX, pyriprole+SX, pyriproxyfen+SX, quinalphos+SX, resmethrin+SX, rotenone+SX, ryanodine+SX, sarolaner+SX, selamectin+SX, sigma-cypermethrin+SX, silafluofen+SX, sodium borate+SX, sodium metaborate+SX, spinetoram+SX, spinosad+SX, spirodiclofen+SX, spiromesifen+SX, spiropidion+SX, spirotetramat+SX, sulfluramid+SX, sulfotep+SX, sulfoxaflor+SX, sulfur+SX, sulfuryl fluoride+SX, tartar emetic+SX, tau-fluvalinate+SX, tebufenozide+SX, tebufenpyrad+SX, tebupirimfos+SX, teflubenzuron+SX, tefluthrin+SX, temephos+SX, terbufos+SX, terpene constituents of the extract of Chenopodium ambrosioides near ambrosioides+SX, tetrachlorantraniliprole+SX, tetrachlorvinphos+SX, tetradifon+SX, tetramethrin+SX, tetramethylfluthrin+SX, tetraniliprole+SX, theta-cypermethrin+SX, thiacloprid+SX, thiamethoxam+SX, thiocyclam+SX, thiodicarb+SX, thiofanox+SX, thiometon+SX, thiosultap-disodium+SX, thiosultap-monosodium+SX, tioxazafen+SX, tolfenpyrad+SX, tralomethrin+SX, transfluthrin+SX, triazamate+SX, triazophos+SX, trichlorfon+SX, 1 triflumezopyrim+SX, triflumuron+SX, trimethacarb+SX, tyclopyrazoflor+SX, vamidothion+SX, wood extract of Quassia amara+SX, XMC (3,5-dimethylphenyl N-methylcarbamate)+SX, xylylcarb+SX, zeta-cypermethrin+SX, zinc phosphide+SX, N-[3-chloro-1-(pyridin-3-yl)-1H-pyrazol-4-yl]-N-ethyl-3-(3,3,3-trifluoropropanesulfinyl)propanamide (1477923-37-7)+SX, 4-[5-(3,5-dichlorophenyl)-5-(trifluoromethyl)-4,5-dihydro-1,2-oxazol-3-yl]-2-methyl-N-(1-oxothietan-3-yl)benzamide (1241050-20-3)+SX, 3-methoxy-N-(5-{5-(trifluoromethyl)-5-[3-(trifluoromethyl)phenyl]-4,5-dihydro-1,2-oxazol-3-yl}indan-1-yl)propanamide (1118626-57-5)+SX, N-[2-bromo-6-chloro-4-(1,1,1,2,3,3,3-heptafluoropropan-2-yl)phenyl]-3-{ethyl[(pyridin-4-yl)carbonyl]amino}-2-methoxybenzamide (1429513-53-0)+SX, N-[2-bromo-6-chloro-4-(1,1,1,2,3,3,3-heptafluoropropan-2-yl)phenyl]-3-[ethyl(4-cyanobenzoyl)amino]-2-methoxybenzamide (1609007-65-9)+SX, N-[2-bromo-6-(difluoromethoxy)-4-(1,1,1,2,3,3,3-heptafluoropropan-2-yl)phenyl]-3-{methyl[(pyridin-4-yl)carbonyl]amino}-2-methoxybenzamide (1630969-78-6)+SX, 1-{2-fluoro-4-methyl-5-[(2,2,2-trifluoroethyl)sulfanyl]phenyl}-3-(trifluoromethyl)-1H-1,2,4-triazol-5-amine (885026-50-6)+SX, BT crop protein Cry1Ab+SX, BT crop protein Cry1Ac+SX, BT crop protein Cry1Fa+SX, BT crop protein Cry1A105+SX, BT crop protein Cry2Ab+SX, BT crop protein Vip3A+SX, BT crop protein Cry3A+SX, BT crop protein Cry3Ab+SX, BT crop protein Cry3Bb+SX, BT crop protein Cry34AB1/Cry35AB1+SX, Adoxophyes orana granulosis virus strain BV-0001+SX, Anticarsia gemmatalis mNPV+SX, Autographa californica mNPV+SX, Cydia pomonella GV strain V15+SX, Cydia pomonella GV strain V22+SX, Cryptophlebia leucotreta GV+SX, Dendrolimus punctatus cypovirus+SX, Helicoverpa armigera NPV strain BV-0003+SX, Helicoverpa zea NPV+SX, Lymantria dispar NPV+SX, Mamestra brassicae NPV+SX, Mamestra configurata NPV+SX, Neodiprion abietis NPV+SX, Neodiprion lecontei NPV+SX, Neodiprion sertifer NPV+SX, Nosema locustae+SX, Orgyia pseudotsugata NPV+SX, Pieris rapae GV+SX, Plodia interpunctella GV+SX, Spodoptera exigua mNPV+SX, Spodoptera littoralis mNPV+SX, Spodoptera litura NPV+SX, Arthrobotrys dactyloides+SX, Bacillus firmus strain GB126+SX, Bacillus firmus strain I-1582+SX, Bacillus megaterium+SX, Bacillus sp. strain AQ175+SX, Bacillus sp. strain AQ177+SX, Bacillus sp. strain AQ178+SX, Bacillus sphaericus strain 2362+SX, Bacillus sphaericus strain ABTS1743+SX, Bacillus sphaericus Serotype strain H5a5b+SX, Bacillus thuringiensis strain AQ52+SX, Bacillus thuringiensis strain BD #32+SX, Bacillus thuringiensis strain CR37 1+SX, Bacillus thuringiensis subsp. Aizawai strain ABTS-1857+SX, Bacillus thuringiensis subsp. Aizawai strain AM65-52+SX, Bacillus thuringiensis subsp. Aizawai strain GC-91+SX, Bacillus thuringiensis subsp. Aizawai Serotype strain H-7+SX, Bacillus thuringiensis subsp. Kurstaki strain ABTS351+SX, Bacillus thuringiensis subsp. Kurstaki strain BMP123+SX, Bacillus thuringiensis subsp. Kurstaki strain EG²³⁴+SX, Bacillus thuringiensis subsp. Kurstaki strain EG⁷⁸⁴¹+SX, Bacillus thuringiensis subsp. Kurstaki strain EVB113-19+SX, Bacillus thuringiensis subsp. Kurstaki strain F810+SX, Bacillus thuringiensis subsp. Kurstaki strain HD-1+SX, Bacillus thuringiensis subsp. Kurstaki strain PB54+SX, Bacillus thuringiensis subsp. Kurstaki strain SA11+SX, Bacillus thuringiensis subsp. Kurstaki strain SA12+SX, Bacillus thuringiensis subsp. Tenebriosis strain NB176+SX, Bacillus thuringiensis subsp. Thuringiensis strain MPPL002+SX, Bacillus thuringiensis subsp.morrisoni+SX, Bacillus thuringiensis var. colmeri+SX, Bacillus thuringiensis var. darmstadiensis strain 24-91+SX, Bacillus thuringiensis var. dendrolimus+SX, Bacillus thuringiensis var. galleriae+SX, Bacillus thuringiensis var. israelensis strain BMP144+SX, Bacillus thuringiensis var. israelensis serotype strain H-14+SX, Bacillus thuringiensis var. japonensis strain buibui+SX, Bacillus thuringiensis var. san diego strain M-7+SX, Bacillus thuringiensis vaR7216+SX, Bacillus thuringiensis var. aegypti+SX, Bacillus thuringiensis var. T36+SX, Beauveria bassiana strain ANT-03+SX, Beauveria bassiana strain ATCC74040+SX, Beauveria bassiana strain GHA+SX, Beauveria brongniartii+SX, Burkholderia rinojensis strain A396+SX, Chromobacterium subtsugae strain PPAA4-1T+SX, Dactyllela ellipsospora+SX, Dectylaria thaumasia+SX, Hirsutella minnesotensis+SX, Hirsutella rhossiliensis+SX, Hirsutella thompsonii+SX, Lagenidium giganteum+SX, Lecanicillium lecanii strain KV01+SX, Lecanicillium lecanii conidia of strain DAOM198499+SX, Lecanicillium lecanii conidia of strain DAOM216596+SX, Lecanicillium muscarium strain Ve6+SX, Metarhizium anisopliae strain F52+SX, Metarhizium anisopliae var. acridum+SX, Metarhizium anisopliae var. anisopliae BIPESCO 5/F52+SX, Metarhizium flavoviride+SX, Monacrosporium phymatopagum+SX, Paecilomyces fumosoroseus Apopka strain 97+SX, Paecilomyces lilacinus strain 251+SX, Paecilomyces tenuipes strain T1+SX, Paenibacillus popilliae+SX, Pasteuria nishizawae strain Pnl+SX, Pasteuria penetrans+SX, Pasteuria usgae+SX, Pasteuria thoynei+SX, Serratia entomophila+SX, Verticillium chlamydosporium+SX, Verticillium lecani strain NCIM1312+SX, 2-chloro-4-fluoro-5-{[5-(trifluoromethylthio)pentyl]oxy}phenyl 2,2,2-trifluoroethyl sulfoxide (1472050-04-6)+SX, 4-chloro-5-[2,2-difluoro-2-(3,4,5-trifluorophenyl)ethoxy]-2-methylphenyl 2,2,2-trifluoroethyl sulfoxide (1632218-00-8)+SX, 4-fluoro-5-[2,2-difluoro-2-(3,4,5-trifluorophenyl)ethoxy]-2-methylphenyl 2,2,2-trifluoroethyl sulfoxide (1632217-98-1)+SX, 2-({2-fluoro-4-methyl-5-[(2,2,2-trifluoroethyl)sulfinyl]phenyl}imino)-3-(2,2,2-trifluoroethyl)-1,3-thiazolidin-4-one (1445683-71-5)+SX, (1Z)-2-(4-tert-butylphenyl)-2-cyano-1-(1-ethyl-3-methyl-1H-pyrazol-5-yl)ethenyl 2,2-dimethylpropanoate (1253429-01-4)+SX, N-[(1S,2S)-2-(2,4-dichlorophenyl)cyclobutyl]-2-(trifluoromethyl)pyridine-3-carboxamide (1644251-74-0)+SX, (3R)-3-(2-chlorothiazol-5-yl)-8-methyl-7-oxo-6-phenyl-2,3-dihydrothiazolo[3,2-a]pyrimidin-4-ium-5-olate (2249718-27-0)+SXₒ

Combination of the Present ingredient of the above Group (b) and the Present compound X:
acibenzolar-S-methyl+SX, aldimorph+SX, ametoctradin+SX, aminopyrifen+SX, amisulbrom+SX, anilazine+SX, azaconazole+SX, azoxystrobin+SX, basic copper sulfate+SX, benalaxyl+SX, benalaxyl-M+SX, benodanil+SX, benomyl+SX, benthiavalicarb+SX, benthivalicarb-isopropyl+SX, benzovindiflupyr+SX, binapacryl+SX, biphenyl+SX, bitertanol+SX, bixafen+SX, blasticidin-S+SX, Bordeaux mixture+SX, boscalid+SX, bromothalonil+SX, bromuconazole+SX, bupirimate+SX, captafol+SX, captan+SX, carbendazim+SX, carboxin+SX, carpropamid+SX, chinomethionat+SX, chitin+SX, chloroneb+SX, chlorothalonil+SX, chlozolinate+SX, colletochlorin B+SX, copper(II) acetate+SX, copper(II) hydroxide+SX, copper oxychloride+SX, copper(II) sulfate+SX, coumoxystrobin+SX, cyazofamid+SX, cyflufenamid+SX, cymoxanil+SX, cyproconazole+SX, cyprodinil+SX, dichlobentiazox+SX, dichlofluanid+SX, diclocymet+SX, diclomezine+SX, dicloran+SX, diethofencarb+SX, difenoconazole+SX, diflumetorim+SX, imethachlone+SX, dimethirimol+SX, dimethomorph+SX, dimoxystrobin+SX, diniconazole+SX, diniconazole-M+SX, dinocap+SX, dipotassium hydrogenphosphite+SX, dipymetitrone+SX, dithianon+SX, dodecylbenzenesulphonic acid bisethylenediamine copper(II) salt+SX, dodemorph+SX, dodine+SX, edifenphos+SX, enoxastrobin+SX, epoxiconazole+SX, etaconazole+SX, ethaboxam+SX, ethirimol+SX, etridiazole+SX, extract from Melaleuca alternifolia+SX, extract from Reynoutria sachalinensis+SX, extract from the cotyledons of lupine plantlets(“BLAD”)+SX, extract of Allium sativum+SX, extract of Equisetum arvense+SX, extract of Tropaeolum majus+SX, famoxadone+SX, fenamidone+SX, fenaminstrobin+SX, fenarimol+SX, fenbuconazole+SX, fenfuram+SX, fenhexamid+SX, fenoxanil+SX, fenpiclonil+SX, fenpicoxamid+SX, fenpropidin+SX, fenpropimorph+SX, fenpyrazamine+SX, fentin acetate+SX, fentin chloride+SX, fentin hydroxide+SX, ferbam+SX, ferimzone+SX, florylpicoxamid+SX, fluazinam+SX, fludioxonil+SX, flufenoxystrobin+SX, fluindapyr+SX, flumorph+SX, fluopicolide+SX, fluopyram+SX, fluopimomide+SX, fluoroimide+SX, fluoxapiprolin+SX, fluoxastrobin+SX, fluquinconazole+SX, flusilazole+SX, flusulfamide+SX, flutianil+SX, flutolanil+SX, flutriafol+SX, fluxapyroxad+SX, folpet+SX, fosetyl+SX, fosetyl-aluminium+SX, fuberidazole+SX, furalaxyl+SX, furametpyr+SX, guazatine+SX, hexaconazole+SX, hymexazole+SX, imazalil+SX, imibenconazole+SX, iminoctadine+SX, iminoctadine triacetate+SX, inpyrfluxam+SX, iodocarb+SX, ipconazole+SX, ipfentrifluconazole+SX, ipflufenoquin+SX, iprobenfos+SX, iprodione+SX, iprovalicarb+SX, isofetamid+SX, isoflucypram+SX, isoprothiolane+SX, isopyrazam+SX, isotianil+SX, kasugamycin+SX, kresoxim-methyl+SX, laminarin+SX, leaves and bark of Quercus+SX, mancozeb+SX, mandestrobin+SX, mandipropamid+SX, maneb+SX, mefentrifluconazole+SX, mepanipyrim+SX, mepronil+SX, meptyldinocap+SX, metalaxyl+SX, metalaxyl-M+SX, metconazole+SX, methasulfocarb+SX, metiram+SX, metominostrobin+SX, metrafenone+SX, metyltetraprole+SX, mineral oils+SX, myclobutanil+SX, naftifine+SX, nuarimol+SX, octhilinone+SX, ofurace+SX, orysastrobin+SX, oxadixyl+SX, oxathiapiprolin+SX, oxine-copper+SX, oxolinic acid+SX, oxpoconazole+SX, oxpoconaZole fumarate+SX, oxycarboxin+SX, oxytetracycline+SX, pefurazoate+SX, penconazole+SX, pencycuron+SX, penflufen+SX, penthiopyrad+SX, phenamacril+SX, phosphorous acid+SX, phthalide+SX, picarbutrazox+SX, picoxystrobin+SX, piperalin+SX, polyoxins+SX, potassium hydrogencarbonate+SX, potassium dihydrogenphosphite+SX, probenazole+SX, prochloraz+SX, procymidone+SX, propamidine+SX, propamocarb+SX, propiconazole+SX, propineb+SX, proquinazid+SX, prothiocarb+SX, prothioconazole+SX, pydiflumetofen+SX, pyraclostrobin+SX, pyrametostrobin+SX, pyraoxystrobin+SX, pyrapropoyne+SX, pyraziflumid+SX, pyrazophos+SX, pyribencarb+SX, pyributicarb+SX, pyridachlometyl+SX, pyrifenox+SX, pyrimethanil+SX, pyrimorph+SX, pyriofenone+SX, pyrisoxazole+SX, pyroquilon+SX, Quillaja extract+SX, quinconazole+SX, quinofumelin+SX, quinoxyfen+SX, quintozene+SX, Saponins of Chenopodium quinoa+SX, sedaxane+SX, silthiofam+SX, simeconazole+SX, sodium hydrogencarbonate+SX, spiroxamine+SX, streptomycin+SX, sulfur+SX, tebuconazole+SX, tebufloquin+SX, teclofthalam+SX, tecnazene+SX, terbinafine+SX, tetraconazole+SX, thiabendazole+SX, thifluzamide+SX, thiophanate+SX, thiophanate-methyl+SX, thiram+SX, thymol+SX, tiadinil+SX, tolclofos-methyl+SX, tolfenpyrad+SX, tolprocarb+SX, tolylfluanid+SX, triadimefon+SX, triadimenol+SX, triazoxide+SX, triclopyricarb+SX, tricyclazole+SX, tridemorph+SX, trifloxystrobin+SX, triflumizole+SX, triforine+SX, triticonazole+SX, validamycin+SX, valifenalate+SX, vinclozolin+SX, yellow mustard powder+SX, zinc thiazole+SX, zineb+SX, ziram+SX, zoxamide+SX, N′-[4-({3-[(4-chlorophenyl)methyl]-1,2,4-thiadiazol-5-yl}oxy)-2,5-dimethylphenyl]-N-ethyl-N-methylmethanimidamide (1202781-91-6)+SX, 4-(2-bromo-4-fluorophenyl)-N-(2-chloro-6-fluorophenyl)-1,3-dimethyl-1H-pyrazol-5-amine (1362477-26-6)+SX, 2-[6-(3-fluoro-4-methoxyphenyl)-5-methylpyridin-2-yl]quinazoline (1257056-97-5)+SX, 5-fluoro-2-[(4-methylphenyl)methoxy]pyrimidin-4-amine (1174376-25-0)+SX, 5-fluoro-4-imino-3-methyl-1-tosyl-3,4-dihydropyrimidin-2(1H)-one (1616664-98-2)+SX, N′-(2,5-dimethyl-4-phenoxyphenyl)-N-ethyl-N-methylmethanimidamide (1052688-31-9)+SX, N′-{4-[(4,5-dichlorothiazol-2-yl)oxy]-2,5-dimethylphenyl}-N-ethyl-N-methylmethanimidamide (929908-57-6)+SX, ethyl (2Z)-3-amino-2-cyano-3-phenylacrylate (39491-78-6)+SX, N-[(2-chlorothiazol-5-yl)methyl]-N-ethyl-6-methoxy-3-nitropyridin-2-amine (1446247-98-8)+SX, 5-(4-chlorobenzyl)-2-(chloromethyl)-2-methyl-1-(1H-1,2,4-triazol-1-ylmethyl)cyclopentan-1-ol (1394057-11-4)+SX, (1R, 2S, 5S)-5-(4-chlorobenzyl)-2-(chloromethyl)-2-methyl-1-(1H-1,2,4-triazol-1-ylmethyl)cyclopentan-1-ol (1801930-06-2)+SX, (1S, 2R, 5R)-5-(4-chlorobenzyl)-2-(chloromethyl)-2-methyl-1-(1H-1,2,4-triazol-1-ylmethyl)cyclopentan-1-ol (1801930-07-3)+SX, 2-(chloromethyl)-5-(4-fluorobenzyl)-2-methyl-1-(1H-1,2,4-triazol-1-ylmethyl)cyclopentan-i-ol (1394057-13-6)+SX, (1R, 2S, 5S)-2-(chloromethyl)-5-(4-fluorobenzyl)-2-methyl-1-(1H-1,2,4-triazol-1-ylmethyl)cyclopentan-1-ol (1801930-08-4)+SX, (1S, 2R, 5R)-2-(chloromethyl)-5-(4-fluorobenzyl)-2-methyl-1-(1H-1,2,4-triazol-1-ylmethyl)cyclopentan-1-ol (1801930-09-5)+SX, methyl 3-[(4-chlorophenyl)methyl]-2-hydroxy-1-methyl-2-(1H-1,2,4-triazol-1-ylmethyl)cyclopentan-i-carboxylate (1791398-02-1)+SX, methyl ({2-methyl-5-[1-(4-methoxy-2-methylphenyl)-1H-pyrazol-3-yl]phenyl}methyl)carbamate (1605879-98-8)+SX, 2-(difluoromethyl)-N-[1,1,3-trimethyl-2,3-dihydro-1H-inden-4-yl]pyridine-3-carboxamide (1616239-21-4)+SX, 2-(difluoromethyl)-N-[3-ethyl-1,1-dimethyl-2,3-dihydro-1H-inden-4-yl]pyridine-3-carboxamide (1847460-02-9)+SX, 2-(difluoromethyl)-N-[3-propyl-1,1-dimethyl-2,3-dihydro-1H-inden-4-yl]pyridine-3-carboxamide (1847460-05-2)+SX, (2E,3Z)-5-{[1-(4-chlorophenyl)-1H-pyrazol-3-yl]oxy}-2-(methoxyimino)-N,3-dimethylpent-3-enamide (1445331-27-0)+SX, Agrobacterium radiobactor strain K1026+SX, Agrobacterium radiobactor strain K84+SX, Bacillus amyloliquefaciens (Aveo(Trade mark) EZ Nematicide)+SX, Bacillus amyloliquefaciens strain AT332+SX, Bacillus amyloliquefaciens strain B³+SX, Bacillus amyloliquefaciens strain D747+SX, Bacillus amyloliquefaciens strain DB101+SX, Bacillus amyloliquefaciens strain DB102+SX, Bacillus amyloliquefaciens strain GB03+SX, Bacillus amyloliquefaciens strain FZB24+SX, Bacillus amyloliquefaciens strain FZB42+SX, Bacillus amyloliquefaciens strain IN937a+SX, Bacillus amyloliquefaciens strain MBI600+SX, Bacillus amyloliquefaciens strain QST713+SX, Bacillus amyloliquefaciens isolate strain B²⁴⁶+SX, Bacillus amyloliquefaciens strain F727+SX, Bacillus amyloliquefaciens subsp. plantarum strain D747+SX, Bacillus licheniformis strain HB2+SX, Bacillus licheniformis strain SB3086+SX, Bacillus pumilus strain AQ717+SX, Bacillus pumilus strain BUF-33+SX, Bacillus pumilus strain GB34+SX, Bacillus pumilus strain QST2808+SX, Bacillus simplex strain CGF2856+SX, Bacillus subtilis strain AQ153+SX, Bacillus subtilis strain AQ743+SX, Bacillus subtilis strain BU1814+SX, Bacillus subtilis strain D747+SX, Bacillus subtilis strain DB101+SX, Bacillus subtilis strain FZB24+SX, Bacillus subtilis strain GB03+SX, Bacillus subtilis strain HAI0404+SX, Bacillus subtilis strain IAB/BS03+SX, Bacillus subtilis strain MBI600+SX, Bacillus subtilis strain QST30002/AQ30002+SX, Bacillus subtilis strain QST30004/AQ30004+SX, Bacillus subtilis strain QST713+SX, Bacillus subtilis strain QST714+SX, Bacillus subtilis var. Amyloliquefaciens strain FZB24+SX, Bacillus subtilis strain Y1336+SX, Burkholderia cepacia+SX, Burkholderia cepacia type Wisconsin strain J82+SX, Burkholderia cepacia type Wisconsin strain M54+SX, Candida oleophila strain 0+SX, Candida saitoana+SX, Chaetomium cupreum+SX, Clonostachys rosea+SX, Coniothyrium minitans strain CGMCC8325+SX, Coniothyrium minitans strain CON/M/91-8+SX, cryptococcus albidus+SX, Erwinia carotovora subsp.carotovora strain CGE234M403+SX, Fusarium oxysporum strain Fo47+SX, Gliocladium catenulatum strain J1446+SX, Paenibacillus polymyxa strain AC-1+SX, Paenibacillus polymyxa strain BS-0105+SX, Pantoea agglomerans strain E325+SX, Phlebiopsis gigantea strain VRA1992+SX, Pseudomonas aureofaciens strain TX-1+SX, Pseudomonas chlororaphis strain 63-28+SX, Pseudomonas chlororaphis strain AFS009+SX, Pseudomonas chlororaphis strain MA342+SX, Pseudomonas fluorescens strain 1629RS+SX, Pseudomonas fluorescens strain A⁵⁰⁶+SX, Pseudomonas fluorescens strain CL145A+SX, Pseudomonas fluorescens strain G⁷⁰⁹⁰+SX, Pseudomonas sp. strain CAB-02+SX, Pseudomonas syringae strain 742RS+SX, Pseudomonas syringae strain MA-4+SX, Pseudozyma flocculosa strain PF-A²²UL+SX, Pseudomonas rhodesiae strain HAI-0804+SX, Pythium oligandrum strain DV74+SX, Pythium oligandrum strain M1+SX, Streptomyces griseoviridis strain K61+SX, Streptomyces lydicus strain WYCD108US+SX, Streptomyces lydicus strain WYEC108+SX, Talaromyces flavus strain SAY-Y-94-01+SX, Talaromyces flavus strain V117b+SX, Trichoderma asperellum strain ICCO12+SX, Trichoderma asperellum SKT-1+SX, Trichoderma asperellum strain T25+SX, Trichoderma asperellum strain T34+SX, Trichoderma asperellum strain TV1+SX, Trichoderma atroviride strain CNCM 1-1237+SX, Trichoderma atroviride strain LC52+SX, Trichoderma atroviride strain IMI 206040+SX, Trichoderma atroviride strain SCl+SX, Trichoderma atroviride strain SKT-1+SX, Trichoderma atroviride strain T11+SX, Trichoderma gamsii strain ICC080+SX, Trichoderma harzianum strain 21+SX, Trichoderma harzianum strain DB104+SX, Trichoderma harzianum strain DSM 14944+SX, Trichoderma harzianum strain ESALQ-1303+SX, Trichoderma harzianum strain ESALQ-1306+SX, Trichoderma harzianum strain IIHR-Th-2+SX, Trichoderma harzianum strain ITEM908+SX, Trichoderma harzianum strain kd+SX, Trichoderma harzianum strain MO1+SX, Trichoderma harzianum strain SF+SX, Trichoderma harzianum strain T22+SX, Trichoderma harzianum strain T39+SX, Trichoderma harzianum strain T78+SX, Trichoderma harzianum strain TH35+SX, Trichoderma polysporum strain IMI206039+SX, trichoderma stromaticum+SX, Trichoderma virens strain G⁴¹+SX, Trichoderma virens strain GL-21+SX, Trichoderma viride+SX, Variovorax paradoxus strain CGF4526+SX, Harpin protein+SX, N′-[5-choro-4-(2-fluorophenoxy)-2-methylphenyl)-N-ethyl-N-methylmethanimidamide (2055589-28-9)+SX, N′-[2-choro-4-(2-fluorophenoxy)-5-methylphenyl)-N-ethyl-N-methylmethanimidamide (2055756-21-1)+SX, N′-[4-(1-hydroxy-1-phenyl-2,2,2-trifluoroethyl)-2-methyl-5-methoxyphenyl)-N-isopropyl-N-methylmethanimidamide (2101814-55-3)+SX, N′-[5-bromo-6-(1-methyl-2-propoxyethoxy)-2-methylpyridin-3-yl)-N-ethyl-N-methylmethanimidamide (1817828-69-5)+SX, 1-(2,4-difluorophenyl)-2-(1H-1,2,4-triazol-1-yl)-1-[1-(4-bromo-2,6-difluorophenoxy)cyclopropyl]ethanol (2019215-86-0)+SX, 1-(2,4-difluorophenyl)-2-(1H-1,2,4-triazol-1-yl)-1-[1-(4-chloro-2,6-difluorophenoxy)cyclopropyl]ethanol (2019215-84-8)+SX, 1-[2-(1-chlorocyclopropyl)-3-(2-fluorophenyl)-2-hydroxypropyl]-1H-imidazole-5-carbonitrile (2018316-13-5)+SX, 1-[2-(1-chlorocyclopropyl)-3-(2,3-difluorophenyl)-2-hydroxypropyl]-1H-imidazole-5-carbonitrile (2018317-25-2)+SX, 4-({6-[2-(2,4-difluorophenyl)-1,1-difluoro-2-hydroxy-3-(1H-1,2,4-triazol-1-yl)propyl]pyridin-3-yl}oxy)benzonitrile (2046300-61-0)+SX, 2-[6-(4-bromophenoxy)-2-(trifluoromethyl)pyridin-3-yl]-1-(1H-1,2,4-triazol-1-yl)propan-2-ol (2082661-43-4)+SX, 2-[6-(4-chlorophenoxy)-2-(trifluoromethyl)pyridin-3-yl]-1-(1H-1,2,4-triazol-1-yl)propan-2-ol (2082660-27-1)+SX, (2E,3Z)-5-{[1-(2,4-dichlorophenyl)-1H-pyrazol-3-yl]oxy}-2-(methoxyimino)-N,3-dimethylpent-3-enamide (1445331-54-3)+SX, 5-chloro-4-({2-[6-(4-chlorophenoxy)pyridin-3-yl]ethyl}amino)-6-methylpyrimidine (1605340-92-8)+SX, N-(1-benzyl-1,3-dimethylbutyl)-8-fluoroquinoline-3-carboxamide (2132414-04-9), N-(1-benzyl-3,3,3-trifluoro-1-methylpropyl)-8-fluoroquinoline-3-carboxamide (2132414-00-5)+SX, 4,4-dimethyl-2-({4-[5-(trifluoromethyl)-1,2,4-oxadiazol-3-yl]phenyl}methyl)isoxaZolidin-3-one (2098918-25-1)+SX, 5,5-dimethyl-2-({4-[5-(trifluoromethyl)-1,2,4-oxadiazol-3-yl]phenyl}methyl)isoxazolidin-3-one (2098918-26-2)+SX.

Combination of the Present ingredient of the above Group (c) and the Present compound X:

1-methylcyclopropene+SX, 1,3-diphenylurea+SX, 2,3,5-triiodobenzoic acid+SX, IAA ((1H-indol-3-yl)acetic acid)+SX, IBA (4-(1H-indol-3-yl)butyric acid)+SX, MCPA (2-(4-chloro-2-methylphenoxy)acetic acid)+SX, MCPB (4-(4-chloro-2-methylphenoxy)butyric acid)+SX, 4-CPA (4-chlorophenoxyacetic acid)+SX, 5-aminolevulinic acid hydrochloride+SX, 6-benzylaminopurine+SX, abscisic acid+SX, AVG (aminoethoxyvinylglycine)+SX, ancymidol+SX, butralin+SX, calcium carbonate+SX, calcium chloride+SX, calcium formate+SX, calcium peroxide+SX, calcium polysulfide+SX, calcium sulfate+SX, chlormequat-chloride+SX, chlorpropham+SX, choline chloride+SX, cloprop+SX, cyanamide+SX, cyclanilide+SX, daminozide+SX, decan-1-ol+SX, dichlorprop+SX, dikegulac+SX, dimethipin+SX, diquat+SX, ethephon+SX, ethychlozate+SX, flumetralin+SX, flurprimidol+SX, forchlorfenuron+SX, formononetin+SX, Gibberellin A+SX, Gibberellin A3+SX, inabenfide+SX, Kinetin+SX, lipochitooligosaccharide SP104+SX, maleic hydrazide+SX, mefluidide+SX, mepiquat-chloride+SX, oxidized glutathione+SX, pacrobutrazol+SX, pendimethalin+SX, prohexandione-calcium+SX, prohydrojasmon+SX, pyraflufen-ethyl+SX, sintofen+SX, sodium 1-naphthaleneacetate+SX, sodium cyanate+SX, streptmycin+SX, thidiazuron+SX, triapenthenol+SX, Tribufos+SX, trinexapac-ethyl+SX, uniconazole-P+SX, 2-(naphthalen-1-yl)acetamide+SX, [4-oxo-4-(2-phenylethyl)amino]butyric acid+SX, methyl 5-(trifluoromethyl)benzo[b]thiophene-2-carboxylate+SX, 3-[(6-chloro-4-phenylquinazolin-2-yl)amino]-1-propanol+SX, Claroideoglomus etunicatum+SX, Claroideoglomus claroideum+SX, Funneliformis mosseae+SX, Gigaspora margarita+SX, Gigaspora rosea+SX, Glomus aggregatum+SX, Glomus deserticola+SX, Glomus monosporum+SX, Paraglomus brasillianum+SX, Rhizophagus clarus+SX, Rhizophagus intraradices RTI-801+SX, Rhizophagus irregularis DAOM 197198+SX, Azorhizobium caulinodans+SX, Azospirillum amazonense+SX, Azospirillum brasilense XOH+SX, Azospirillum brasilense Ab-V5+SX, Azospirillum brasilense Ab-V6+SX, Azospirillum caulinodans+SX, Azospirillum halopraeferens+SX, Azospirillum irakense+SX, Azospirillum lipoferum+SX, Bradyrhizobium elkanii SEMIA 587+SX, Bradyrhizobium elkanii SEMIA 5019+SX, Bradyrhizobium japonicum TAll+SX, Bradyrhizobium japonicum USDA110+SX, Bradyrhizobium liaoningense+SX, Bradyrhizobium lupini+SX, Delftia acidovorans RAY209+SX, Mesorhizobium ciceri+SX, Mesorhizobium huakii+SX, Mesorhizobium loti+SX, Rhizobium etli+SX, Rhizobium galegae+SX, Rhizobium leguminosarum bv. Phaseoli+SX, Rhizobium leguminosarum bv. Trifolii+SX, Rhizobium leguminosarum bv. Viciae+SX, Rhizobium trifolii+SX, Rhizobium tropici+SX, Sinorhizobium fredii+SX, Sinorhizobium meliloti+SX, Zucchini Yellow Mosaik Virus weak strain+SX.

Combination of the Present ingredient of the above Group (d) and the Present compound X:

anthraquinone+SX, deet+SX, icaridin+SX.

The ratio of the Present compound X to the Present ingredient includes, but not limited thereto, as a ratio by weight (the Present compound X:the Present ingredient) 1,000:1 to 1:1,000, 500:1 to 1:500, 100:1 to 1:100, 50:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, and 1:50, and the others.

Examples of the pest on which the Present compound X has control efficacies include harmful arthropods such as harmful insects, harmful mites, harmful nematodes, and harmful mollusks. Specific examples of the pest include, but are not limited to, the followings.

### Hemiptera Pests:

from the family Delphacidae, for example, small brown planthopper (Laodelphax striatella), brown planthopper (Nilaparvata lugens), white-backed planthopper (Sogatella furcifera), corn planthopper (Peregrinus maidis), cereal leafhopper (Javesella pellucida), sugarcane leafhopper (Perkinsiella saccharicida), and Tagosodes orizicolus; from the family Cicadellidae, for example, green rice leafhopper (Nephotettix cincticeps), green paddy leafhopper (Nephotettix virescens), rice leafhopper (Nephotettix nigropictus), zigzag-striped leafhopper (Recilia dorsalis), tea green leafhopper (Empoasca onukii), potato leafhopper (Empoasca fabae), corn leafhopper (Dalbulus maidis), and rice leafhopper (Cofana spectra);
from the family Cercopidae, for example, Mahanarva posticata, and Mahanarva fimbriolata;
   from the family Aphididae, for example, bean aphid (Aphis fabae), soybean aphid (Aphis glycines), cotton aphid (Aphis gossypii), green apple aphid (Aphis pomi), apple aphid (Aphis spiraecola), green peach aphid (Myzus persicae), leaf-curling plum aphid (Brachycaudus helichrysi), cabbage aphid (Brevicoryne brassicae), rosy apple aphid (Dysaphis plantaginea), false cabbage aphid (Lipaphis erysimi), potato aphid (Macrosiphum euphorbiae), foxglove aphid (Aulacorthum solani), lettuce aphid (Nasonovia ribisnigri), grain aphid (Rhopalosiphum padi), corn aphid (Rhopalosiphum maidis), brown citrus aphid (Toxoptera citricida), mealy plum aphid (Hyalopterus pruni), cane aphid (Melanaphis sacchari), black rice root aphid (Tetraneura nigriabdominalis), sugarcane cottony aphid (Ceratovacuna lanigera), and apple woolly aphid (Eriosoma lanigerum);
from the family Phylloxeridae, for example, grapevine phylloxera (Daktulosphaira vitifoliae), Pecan phylloxera (Phylloxera devastatrix), Pecan leaf phylloxera (Phylloxera notabilis), and Southern pecan leaf phylloxera (Phylloxera russellae);
from the family Adelgidae, for example, hemlock woolly aphid (Adelges tsugae), balsam woolly aphid (Adelges piceae), and Aphrastasia pectinatae;
   from the family Pentatomidae, for example, black rice bug (Scotinophara lurida), Malayan rice black bug (Scotinophara coarctata), common green stink bug (Nezara antennata), white-spotted spined bug (Eysarcoris aeneus), lewis spined bug (Eysarcoris lewisi), white-spotted bug (Eysarcoris ventralis), Eysarcoris annamita, brown marmorated stink bug (Halyomorpha halys), green plant bug (Nezara viridula), brown stink bug (Euschistus heros), red banded stink bug (Piezodorus guildinii), Oebalus pugnax, and Dichelops melacanthus;
from the family Cydnidae, for example, Burrower brown bug (Scaptocoris castanea);
from the family Alydidae, for example, bean bug (Riptortus pedestris), corbett rice bug (Leptocorisa chinensis), and rice bug (Leptocorisa acuta);
from the family Coreidae, for example, Cletus punctiger, and Australian leaf-footed bug (Leptoglossus australis);
from the family Lygaeidae, for example, oriental chinch bug (Caverelius saccharivorus), seed bug (Togo hemipterus), and chinch bug (Blissus leucopterus);
from the family Miridae, for example, rice leaf bug (Trigonotylus caelestialium), sorghum plant bug (Stenotus rubrovittatus), wheat leaf bug (Stenodema calcarata), and American tarnished plant bug (Lygus lineolaris);
from the family Aleyrodidae, for example, greenhouse whitefly (Trialeurodes vaporariorum), tobacco whitefly (Bemisia tabaci), citrus whitefly (Dialeurodes citri), citrus spiny whitefly (Aleurocanthus spiniferus), tea spiny whitefly (Aleurocanthus camelliae), and Pealius euryae; from the family Diaspididae, for example, Abgrallaspis cyanophylli, red scale (Aonidiella aurantii), San Jose scale (Diaspidiotus perniciosus), white peach scale (Pseudaulacaspis pentagona), arrowhead scale (Unaspis yanonensis), and citrus snow scale (Unaspis citri); from the family Coccidae, for example, pink wax scale (Ceroplastes rubens);
from the family Margarodidae, for example, fluted scale (Icerya purchasi) and seychelles fluted scale (Icerya seychellarum);
from the family Pseudococcidae, for example, solanum mealybug (Phenacoccus solani), cotton mealybug (Phenacoccus solenopsis), Japanese mealybug (Planococcus kraunhiae), white peach scale (Pseudococcus comstocki), citrus mealybug (Planococcus citri), currant mealybug (Pseudococcus calceolariae), long-tailed mealybug (Pseudococcus longispinus), and tuttle mealybug (Brevennia rehi); from the family Psyllidae, for example, citrus psylla (Diaphorina citri), two-spotted citrus psyllid (Trioza erytreae), pear sucker (Cacopsylla pyrisuga), Cacopsylla chinensis, potato psyllid (Bactericera cockerelli), and Pear psylla (Cacopsylla pyricola);
from the family Tingidae, for example, sycamore lace bug (Corythucha ciliata), aster tingid (Corythucha marmorata), Japanese pear lace bug (Stephanitis nashi), and azalea lace bug (Stephanitis pyrioides);
from the family Cimicidae, for example, common bed bug (Cimex lectularius);
from the family Cicadidae, for example, Giant Cicada (Quesada gigas);
from Triatoma spp., for example, Triatoma infestans; and the others.

### Lepidoptera Pests:

from the family Crambidae, for example, rice stem borer (Chilo suppressalis), Darkheaded stem borer (Chilo polychrysus), white stem borer (Scirpophaga innotata), yellow paddy borer (Scirpophaga incertulas), Rupela albina, rice leaf roller (Cnaphalocrocis medinalis), Marasmia patnalis, rice leaf roller (Marasmia exigua), cotton leaf roller (Notarcha derogata), corn borer (Ostrinia furnacalis), European corn borer (Ostrinia nubilalis), cabbage webworm (Hellula undalis), grape leafroller (Herpetogramma luctuosale), bluegrass webworm (Pediasia teterrellus), rice case-worm (Nymphula depunctalis), and Sugarcane borer (Diatraea saccharalis);
from the family Pyralidae, for example, lesser cornstalk borer (Elasmopalpus lignosellus) mealworm moth (Plodia interpunctella), and persimmon bark borer (Euzophera batangensis);
from the family Noctuidae, for example, cotton worm (Spodoptera litura), beet armyworm (Spodoptera exigua), rice armyworm (Mythimna separata), cabbage moth (Mamestra brassicae), pink borer (Sesamia inferens), grass armyworm (Spodoptera mauritia), green rice caterpillar (Naranga aenescens), Spodoptera frugiperda, true armyworm (Spodoptera exempta), black cutworm (Agrotis ipsilon), beet worm (Autographa nigrisigna), rice looper (Plusia festucae), soybean looper (Chrysodeixis includens), Trichoplusia spp., Heliothis spp. (such as tobacco budworm (Heliothis virescens)), Helicoverpa spp. (such as tobacco budworm (Helicoverpa armigera) and, corn earworm (Helicoverpa zea)), velvetbean caterpillar (Anticarsia gemmatalis), cotton leafworm (Alabama argillacea), and hop vine borer (Hydraecia immanis);
from the family Pieridae, for example, common cabbage worm (Pieris rapae);
from the family Tortricidae, for example, oriental fruit moth (Grapholita molesta), Grapholita dimorpha, soybean moth (Leguminivora glycinivorella), Matsumuraeses azukivora, summer fruit tortrix (Adoxophyes orana fasciata), smaller tea tortrix (Adoxophyes honmai), Japanese tea tortrix (Homona magnanima), apple tortrix (Archips fuscocupreanus), codling moth (Cydia pomonella), sugarcane shoot borer (Tetramoera schistaceana), bean shoot borer (Epinotia aporema), and citrus fruit borer (Ecdytolopha aurantiana);
from the family Gracillariidae, for example, tea leaf roller (Caloptilia theivora), and Asiatic apple leaf miner (Phyllonorycter ringoniella);
from the family Carposinidae, for example, peach fruit moth (Carposina sasakii);
from the family Lyonetiidae, for example, Coffee leaf miner (Leucoptera coffeella), peach leaf miner (Lyonetia clerkella), and Lyonetia prunifoliella;
from the family Lymantriidae, for example, Lymantria spp. (such as gypsy moth (Lymantria dispar)) and, Euproctis spp. (such as tea lymantriid (Euproctis pseudoconspersa)); from the family Plutellidae, for example, diamondback moth (Plutella xylostella);
from the family Gelechiidae, for example, peach worm (Anarsia lineatella), sweetpotato leaf folder (Helcystogramma triannulella), pink bollworm (Pectinophora gossypiella), potato moth (Phthorimaea operculella), and Tuta absoluta;
from the family Arctiidae, for example, American white moth (Hyphantria cunea);
from the family Castniidae, for example, giant sugarcane borer (Telchin licus);
from the family Cossidae, for example, Cossus insularis;
from the family Geometridae, for example, Ascotis selenaria;
from the family Limacodidae, for example, blue-striped nettle grub (Parasa lepida);
from the family Stathmopodidae, for example, persimmon fruit moth (Stathmopoda masinissa);
from the family Sphingidae, for example, tobacco hornworm (Acherontia lachesis);
from the family Sesiidae, for example, Nokona feralis, cherry borer (Synanthedon hector), and Synanthedon tenuis; from the family Hesperiidae, for example, rice skipper (Parnara guttata);
from the family Tineidae, for example, casemaking clothes moth (Tinea translucens), and common clothes moth (Tineola bisselliella);
   and the others.

### Thysanoptera Pests:

from the family Thripidae, for example, western flower thrips (Frankliniella occidentalis), oriental thrips (Thrips palmi), yellow tea thrips (Scirtothrips dorsalis), onion thrips (Thrips tabaci), eastern flower thrips (Frankliniella intonsa), rice thrips (Stenchaetothrips biformis), and Echinothrips americanus;
from the family Phlaeothripidae, for example, aculeated rice thrips (Haplothrips aculeatus);
   and the others.

### Diptera Pests:

from the family Anthomyiidae, for example, seedcorn maggot (Delia platura), onion maggot (Delia antiqua), and beet leaf miner (Pegomya cunicularia);
from the family Ulidiidae, for example, sugarbeet root maggot (Tetanops myopaeformis);
   from the family Agromyzidae, for example, rice leaf miner (Agromyza oryzae), tomato leaf miner (Liriomyza sativae), chrysanthemum leaf miner (Liriomyza trifolii), and pea leafminer (Chromatomyia horticola);
from the family Chloropidae, for example, rice stem maggot (Chlorops oryzae);
from the family Tephritidae, for example, melon fly (Bactrocera cucurbitae), oriental fruit fly (Bactrocera dorsalis), Malaysian fruit fly (Bactrocera latifrons), olive fruit fly (Bactrocera oleae), Queensland fruit fly (Bactrocera tryoni), Mediterranean fruit fly (Ceratitis capitata), apple maggot (Rhagoletis pomonella), and Japanese cherry fruit fly (Rhacochlaena japonica);
from the family Ephydridae, for example, smaller rice leaf miner (Hydrellia griseola), whorl maggot (Hydrellia philippina), and paddy stem maggot (Hydrellia sasakii); from the family Drosophilidae, for example, cherry drosophila (Drosophila suzukii);
from the family Phoridae, for example, Megaselia spiracularis;
from the family Psychodidae, for example, Clogmia albipunctata;
from the family Sciaridae, for example, Bradysia difformis;
from the family Cecidomyiidae, for example, hessian fly (Mayetiola destructor) and, paddy gall fly (Orseolia oryzae);
from the family Diopsidae, for example, Diopsis macrophthalma;
from the family Tipulidae, for example, rice crane fly (Tipula aino), common cranefly (Tipula oleracea), and European cranefly (Tipula paludosa);
from the family Culicidae, for example, southern house mosquito (Culex pipiens pallens), dengue mosquito (Aedes aegypti), Asian tiger mosquito (Aedes albopictus), Chinese malaria mosquito (Anopheles hyracanus sinensis), Culex quinquefasciatus, Culex pipiens molestus Forskal, and brown house mosquito (Culex quinquefasciatus);
from the family Simulidae, for example, Prosimulium yezoensis, and Simulium ornatum;
from the family Tabanidae, for example, Tabanus trigonus;
from the family Muscidae, for example, house fly (Musca domestica), false stable fly (Muscina stabulans), biting house fly (Stomoxys calcitrans), and buffalo fly (Haematobia irritans);
from the family Tabanidae, for example, Tabanus trigonus;
from the family Calliphoridae;
from the family Sarcophagidae;
from the family Chironomidae, for example, Chironomus plumosus, Chironomus yoshimatsui, and Glyptotendipes tokunagai;
from the family Fannidae;
   and the others.

### Coleoptera Pests:

from the family Chrysomelidae, for example, western corn rootworm (Diabrotica virgifera virgifera), southern corn rootworm (Diabrotica undecimpunctata howardi), northern corn rootworm (Diabrotica barberi), Mexican corn rootworm (Diabrotica virgifera zeae), banded cucumber beetle (Diabrotica balteata), cucurbit beetle (Diabrotica speciosa), bean leaf beetle (Cerotoma trifurcata), barley leaf beetle (Oulema melanopus), cucurbit leaf beetle (Aulacophora femoralis), striped flea beetle (Phyllotreta striolata), cabbage flea beetle (Phyllotreta cruciferae), western black flea beetle (Phyllotreta pusilla), cabbage stem flea beetle (Psylliodes chrysocephala), Colorado potato beetle (Leptinotarsa decemlineata), rice leaf beetle (Oulema oryzae), grape colaspis (Colaspis brunnea), corn flea beetle (Chaetocnema pulicaria), sweet-potato flea beetle (Chaetocnema confinis), potato flea beetle (Epitrix cucumeris), rice leaf beetle (Dicladispa armigera), southern corn leaf beetle (Myochrous denticollis), Laccoptera quadrimaculata, and tobacco flea beetle (Epitrix hirtipennis);
from the family Carabidae, for example, Seedcorn beetle (Stenolophus lecontei), and slender seedcorn beetle (Clivina impressifrons);
   from the family Scarabaeidae, for example, cupreus chafer (Anomala cuprea), soybean beetle (Anomala rufocuprea), Anomala albopilosa, Japanese beetle (Popillia japonica), yellowish elongate chafer (Heptophylla picea), European Chafer (Rhizotrogus majalis), Tomarus gibbosus, Holotrichia spp., Phyllophaga spp. (such as June beetle (Phyllophaga crinita)), and Diloboderus spp. (such as Diloboderus abderus);
from the family Curculionidae, for example, coffee bean weevil (Araecerus coffeae), sweet-potato weevil (Cylas formicarius), West Indian sweet-potato weevil (Euscepes postfasciatus), alfalfa weevil (Hypera postica), maize wevil (Sitophilus zeamais), rice plant weevil (Echinocnemus squameus), rice water weevil (Lissorhoptrus oryzophilus), Rhabdoscelus lineaticollis, boll weevil (Anthonomus grandis), nunting billbug (Sphenophorus venatus), southern corn billbug (Sphenophorus callosus), soybean stalk weevil (Sternechus subsignatus), sugarcane weevil (Sphenophorus levis), rusty gourd-shaped weevil (Scepticus griseus), brown gourd-shaped weevil (Scepticus uniformis), Mexican bean weevil (Zabrotes subfasciatus), pine beetle (Tomicus piniperda), coffee berry borer (Hypothenemus hampei), Aracanthus spp. (such as Aracanthus mourei), and cotton root borer (Eutinobothrus brasiliensis);
from the family Tenebrionidae, for example, red meal beetle (Tribolium castaneum), and mason beetle (Tribolium confusum);
from the family Coccinellidae, for example, twenty-eight-spotted ladybird (Epilachna vigintioctopunctata); from the family Bostrychidae, for example, common powder-post beetle (Lyctus brunneus);
from the family Ptinidae;
from the family Cerambycidae, for example, citrus long-horned beetle (Anoplophora malasiaca) and, Migdolus fryanus; from the family Elateridae, for example, Melanotus okinawensis, barley wireworm (Agriotes fuscicollis), Melanotus legatus, Anchastus spp., Conoderus spp., Ctenicera spp., Limonius spp., and Aeolus spp.;
from the family Staphylinidae, for example, Paederus fuscipes;
from the family Dermestidae, for example, varied carpet beetle (Anthrenus verbasci) and, hide beetle (Dermestes maculates);
from the family Anobiidae, for example, tobacco beetle (Lasioderma serricorne), and biscuit beetle (Stegobium paniceum);
   and the others.

### Orthoptera Pests:

from the family Acrididae, for example, oriental migratory locust (Locusta migratoria), Moroccan locust (Dociostaurus maroccanus), Australian plague locust (Chortoicetes terminifera), red locust (Nomadacris septemfasciata), brown locust (Locustana pardalina), tree locust (Anacridium melanorhodon), Italian locust (Calliptamus italicus), differential grasshopper (Melanoplus differentialis), two-striped grasshopper (Melanoplus bivittatus), migratory grasshopper (Melanoplus sanguinipes), red-legged grasshopper (Melanoplus femurrubrum), clear-winged grasshopper (Camnula pellucida), desert locust (Schistocerca gregaria), Yellow-winged locust (Gastrimargus musicus), spur-throated locust (Austracris guttulosa), Japanese grasshopper (Oxya yezoensis), rice grasshopper (Oxya japonica), and Bombay locust (Patanga succincta); from the family Gryllotalpidae, for example, oriental mole cricket (Gryllotalpa orientalis);
from the family Gryllidae, for example, house cricket (Acheta domestica), and emma field cricket (Teleogryllus emma);
from the family Tettigoniidae, for example, mormon cricket (Anabrus simplex);
   and the others.

### Hymenoptera Pests:

from the family Tenthredinidae, for example, beet sawfly (Athalia rosae), and nippon cabbage sawfly (Athalia japonica);
from the family Formicidae, for example, Solenopsis spp. (such as red imported fire ant (Solenopsis invicta) and, tropical fire ant (Solenopsis geminata)), Atta spp. (such as brown leaf-cutting ant (Atta capiguara)), Acromyrmex spp., Paraponera clavata, black house ant (Ochetellus glaber), little red ant (Monomorium pharaonis), Argentine ant (Linepithema humile), Formica japonica, Pristomyrmex punctutus, Pheidole noda, big-headed ant (Pheidole megacephala), Camponotus spp. (such as Camponotus japonicus, and Camponotus obscuripes), Pogonomyrmex spp. (such as western harvester ant (Pogonomyrmex occidentalis)), Wasmania spp. (such as Wasmania auropunctata), and long-legged ant (Anoplolepis gracilipes);
from the family Vespidae, for example, Asian giant hornet (Vespa mandarinia japonica), Vespa simillima, Vespa analis Fabriciusi, Asian hornet (Vespa velutina), and Polistes jokahamae;
from the family Siricidae, for example, pine wood wasp (Urocerus gigas);
from the family Bethylidae;
   and the others.

### Blattodea Pests:

from the family Blattellidae, for example, German cockroach (Blattella germanica);
from the family Blattidae, for example, smoky-brown cockroach (Periplaneta fuliginosa), American cockroach (Periplaneta americana), brown cockroach (Periplaneta brunnea), and black cockroach (Blatta orientalis); from the family Termitidae, for example, Japanese termite (Reticulitermes speratus), Formosan termite (Coptotermes formosanus), western drywood termite (Incisitermes minor), Cryptotermes domesticus, Odontotermes formosanus, Neotermes koshunensis, Glyptotermes satsumensis, Glyptotermes nakajimai, Glyptotermes fuscus, Hodotermopsis sjostedti, Coptotermes guangzhouensis, Reticulitermes amamianus, Reticulitermes miyatakei, Reticulitermes kanmonensis, Nasutitermes takasagoensis, Pericapritermes nitobei, Sinocapritermes mushae, and Cornitermes cumulans; and the others.

### Siphonaptera Pests:

for example, cat flea (Ctenocephalides felis), dog flea (Ctenocephalides canis), human flea (Pulex irritans), oriental rat flea (Xenopsylla cheopis), chigoe flea (Tunga penetrans), chicken flea (Echidnophaga gallinacea), and European rat flea (Nosopsyllus fasciatus);
   and the others.

### Anoplura Pests:

for example, pig louse (Haematopinus suis), short-nosed cattle louse (Haematopinus eurysternus), sheep biting louse (Dalmalinia ovis), Linognathus seypsus, Pediculus humanis, Pediculuc humanus corporis, Pediculus humanus humanus, and Phthirus pubis;
   and the others.

### Mallophagida Pests:

for example, Bovicola spp. (such as cattle biting louse (Dalmalinia bovis) and, sheep biting louse (Dalmalinia ovis)), Trichodestes spp. (such as dog biting louse (Trichodectes canis)), Felicola spp. (such as cat louse (Felicola subrostrata)), Lipeurus spp. (such as chicken wing louse (Lipeurus caponis)), and Menoponidae family (such as Trimenopon spp. and, Menopon spp.);
   and the others.

### Acari Pests:

from the family Tetranychidae, for example, common red spider mite (Tetranychus urticae), kanzawa spider mite (Tetranychus kanzawai), red spider mite (Tetranychus evansi), citrus red mite (Panonychus citri), fruit-tree red spider mite (Panonychus ulmi), and Oligonychus spp.;
   from the family Eriophyidae, for example, Japanese citrus rust mite (Aculops pelekassi), Phyllocoptruta citri, tomato mite (Aculops lycopersici), purple mite (Calacarus carinatus), tea rust mite (Acaphylla theavagrans), Eriophyes chibaensis, apple bud mite (Aculus schlechtendali), Aceria diospyri, Aceria tosichella, and Shevtchenkella sp.; from the family Tarsonemidae, for example, broad mite (Polyphagotarsonemus latus);
from the family Tenuipalpidae, for example, Brevipalpus phoenicis;
from the family Tuckerellidae;
from the family Ixodidae, for example, Haemaphysalis longicornis, Haemaphysalis flava, Dermacentor taiwanensis, American dog tick (Dermacentor variabilis), Dermacentor andersoni, Ixodes ovatus, Ixodes persulcatus, Ixodes ricinus, black-legged tick (Ixodes scapularis), lone star tick (Amblyomma americanum), Amblyomma maculatum, cattle tick (Boophilus microplus), Boophilus annulatus, and brown dog tick (Rhipicephalus sanguineus);
from the family Acaridae, for example, cereal mite (Tyrophagus putrescentiae), and grassland mite (Tyrophagus similis);
from the family Pyroglyphidae, for example, American house dust mite (Dermatophagoides farinae), and European house dust mite (Dermatophagoides pteronyssinus); from the family Cheyletidae, for example, Cheyletus eruditus, Cheyletus malaccensis, Cheyletus moorei, and Cheyletiella yasguri;
from the family Sarcoptidae, for example, ear mange mite (Otodectes cynotis), and itch mite (Sarcoptes scabiei); from the family Demodicidae, for example, dog follicle mite (Demodex canis);
from the family Listrophoridae;
from the family Haplochthoniidae;
from the family Macronyssidae, for example, tropical rat mite (Ornithonyssus bacoti), and feather mite (Ornithonyssus sylviarum);
from the family Dermanyssidae, for example, bird mite (Dermanyssus gallinae);
from the family Trombiculidae, for example, Leptotrombidium akamushi;
   and the others.

### Araneae Pests:

from the family Eutichuridae, for example, Cheiracanthium japonicum;
from the family Theridiidae, for example, red-back spider (Latrodectus hasseltii);
   and the others.

### Polydesmida:

from the family Paradoxosomatidae, for example, flat-backed millipede (Oxidus gracilis), and Nedyopus tambanus; and the others.

### Isopoda:

from the family Armadillidiidae, for example, common pill bug (Armadillidium vulgare);
   and the others.

### Chilopoda Pests:

from the family Scutigeridae, for example, Thereuonema hilgendorfi;
from the family Scolopendridae, for example, giant tropical centipede (Scolopendra subspinipes);
from the family Ethopolidae, for example, Bothropolys rugosus;
   and the others.

### Gastropoda:

from the family Limacidae, for example, tree slug (Limax marginatus), and garden tawny slug (Limax flavus); from the family Philomycidae, for example, Meghimatium bilineatum;
from the family Ampullariidae, for example, golden apple snail (Pomacea canaliculata);
from the family Lymnaeidae, for example, Austropeplea ollula;
   and the others.

### Nematoda Pests:

from the family Aphelenchoididae, for example, rice white-tip nematode (Aphelenchoides besseyi);
from the family Pratylenchidae, for example, root lesion nematode (Pratylenchus coffeae), Pratylenchus brachyurus, California meadow nematode (Pratylenchus neglectus), and Radopholus similis;
from the family Heteroderidae, for example, javanese root-knot nematode (Meloidogyne javanica), southern root-knot nematode (Meloidogyne incognita), northern root-knot nematode (Meloidogyne hapla), soybean cyst nematode (Heterodera glycines), potato cyst nematode (Globodera rostochiensis), and white potato cyst nematode (Globodera pallida);
from the family Hoplolaimidae, for example, Rotylenchulus reniformis;
from the family Anguinidae, for example, strawberry bud nematode (Nothotylenchus acris), and stem nematode (Ditylenchus dipsaci);
from the family Tylenchulidae, for example, citrus nematode (Tylenchulus semipenetrans);
from the family Longidoridae, for example, dagger nematode (Xiphinema index);
from the family Trichodoridae;
from the family Parasitaphelenchidae, for example, pine wilt disease (Bursaphelenchus xylophilus);
and the others.

As a target, the harmful arthropods such as harmful insects, harmful mites, harmful mollusks, and harmful nematodes may be the harmful arthropods such as harmful insects, harmful mites, harmful mollusks, and harmful nematodes, each of which have a reduced agent-sensitivity to or a developed agent-resistance to an insecticide, a miticide, a molluscicide, and a nematocide, respectively.

The method for controlling harmful arthropods of the present invention is carried out by applying an effective amount of the present compound, the present compound X, or the composition A to a harmful arthropod directly and/or a habitat thereof (for example, plant bodies, soil, an interior of a house, animal bodies). Examples of the method for controlling harmful arthropods of the present invention include foliage treatment, soil treatment, root treatment, shower treatment, smoking treatment, water surface treatment and seed treatment.

The present compound, the present compound X, or the composition A is usually mixed with an inert carrier such as solid carrier, liquid carrier or gaseous carrier, and if necessary, adding surfactants and the other auxiliary agents for formulation, to formulate into emulsifiable concentrates, oil solutions, dust formulations, granules, wettable powders, water dispersible granules, flowables, dry flowables, microcapsules, aerosols, poison baits, resin formulations, shampoo formulations, paste-like formulations, foams, carbon dioxide formulations, and tablets and the others. Such formulations may be processed into mosquito repellent coils, electric mosquito repellent mats, liquid mosquito formulations, smoking agents, fumigants, sheet formulations, spot-on formulations or formulations for oral treatment. These formulations comprise usually 0.0001 to 95% by weight of the present compound, the present compound A or the composition A.

Examples of the solid carrier to be used in the formulation include fine powders or granules of clays (for example, kaolin clay, diatomaceous earth, bentonite, or acid white clay), dry silica, wet silica, talcs, ceramics, other inorganic minerals (for example, sericite, quartz, sulfur, active carbon, or calcium carbonate) or chemical fertilizers (for example, ammonium sulfate, ammonium phosphate, ammonium nitrate, urea, or ammonium chloride) and the others; as well as synthetic resins (for example, polyester resins such as polypropylene, polyacrylonitrile, polymethyl methacrylate or polyethylene terephthalate; nylon resins (for example, nylon-6, nylon-11, or nylon-66); polyamide resins; polyvinyl chloride, polyvinylidene chloride, vinyl chloride-propylene copolymers, and the others).

Examples of the liquid carriers include water; alcohols (for example, methanol, ethanol, isopropyl alcohol, butanol, hexanol, benzyl alcohol, ethylene glycol, propylene glycol, or phenoxy ethanol); ketones (for example, acetone, methyl ethyl ketone, or cyclohexanone); aromatic hydrocarbons (for example, toluene, xylene, ethyl benzene, dodecyl benzene, phenyl xylyl ethane, or methylnaphthalene); aliphatic hydrocarbons (for example, hexane, cyclohexane, kerosene, or light oil); esters (for example, ethyl acetate, butyl acetate, isopropyl myristate, ethyl oleate, diisopropyl adipate, diisobutyl adipate, or propylene glycol monomethyl ether acetate); nitriles (for example, acetonitrile, or isobutyronitrile); ethers (for example, diisopropyl etheR14-dioxane, 1,2-dimethoxyethane, diethyleneglycol dimethyl ether, diethylene glycol monomethyl ether, propylene glycol monomethyl ether, dipropylene glycol monomethyl ether, or 3-methoxy-3-methyl-1-butanol); amides (for example, DMF, or N,N-dimethylacetamide); sulfoxides (for example, dimethyl sulfoxide); propylene carbonate; and vegetable oils (for example, soybean oil or cottonseed oil).

Examples of gaseous carrier include fluorocarbon, butane gas, liquefied petroleum gas (LPG), dimethyl ether, and carbon dioxide gas.

Examples of the surfactants include nonionic surfactants such as polyoxyethylenated alkyl ethers, polyoxyethylenated alkyl aryl ethers, and polyethylene glycol fatty acid esters; and anionic surfactants such as alkyl sulfonates, alkylbenzene sulfonates and alkyl sulfates.

Examples of the other auxiliary agents for formulation include a binder, a dispersant, a colorant and a stabilizer. Specific examples include casein, gelatin, polysaccharides (for example, starch, gum arabic, cellulose derivatives and alginic acid), lignin derivatives, bentonite, water-soluble synthetic polymers (for example, polyvinyl alcohol, polyvinyl pyrrolidone and polyacrylic acids), acidic isopropyl phosphate, 2,6-di-tert-butyl-4-methylphenol, and a mixture of 2-tert-butyl-4-methoxyphenol and 3-tert-butyl-4-methoxyphenol.

Examples of base material of the resin formulation include polyvinyl chloride polymers, polyurethane and the others, and a plasticizer such as phthalate esters (for example, dimethyl phthalate, dioctyl phthalate), adipic acid esters and stearic acid may be added to these base materials, if necessary. The resin formulation can be prepared by mixing the compound of the present invention with the above-mentioned base material, kneading the mixture, followed by molding it by injection molding, extrusion molding or pressure molding and the like. The resultant resin formulation can be subjected to further molding or cutting procedure and the like, if necessary, to be processed into shapes such as a plate, film, tape, net or string shape. These resin formulations can be processed into animal collars, animal ear tags, sheet products, trap strings, gardening supports and other products.

Examples of a base material for the poison baits include bait ingredients such as grain powder, vegetable oil, saccharide and crystalline cellulose, and if necessary, with addition of antioxidants such as dibutylhydroxytoluene and nordihydroguaiaretic acid, preservatives such as dehydroacetic acid, accidental ingestion inhibitors for children and pets such as a chili powder, insect attraction fragrances such as cheese flavor, onion flavor and peanut oil.

The plants as used herein include entire plant, foliages, flowers, ears, fruits, stems, branches, tree canopies, seeds, vegetative reproductive organs, and seedlings.

The vegetative reproductive organs represent a part of plant which have the ability to grow when the part is separated from the body and placed in soil, among the roots, stems, leaves and the like of the plant. Examples of the vegetative reproductive organs include tuberous root, creeping root, bulb, corm or solid bulb, tuber, rhizome, stolon, rhizophore, cane cuttings, propagule, and vine cutting. Here the stolon is also called runner, propagule is also called bulbils, which is divided into broad bud and bulblets. The vines represent shoots (generic name for leaves and stems) of sweet potato and Japanese yam. Discoid stem, corm, tuber, rhizome, stem fragments, rhizophore and tuberous root are also collectively referred to bulbs. For example, though a cultivation of potato begins by planting tubers in soil, the tubers used are generally called seed potatoes.

Examples of a method of controlling harmful arthropods by applying an effective amount of the present compound, the present compound X, or the composition A to soil include a method of applying an effective amount of the present compound, the present compound X, or the composition A to soil before planting plants or after planting plants, a method of applying an effective amount of the present compound, the present compound X, or the composition A to a root part of a crop to be protected from damage such as ingestion by harmful arthropods, and a method of controlling harmful arthropods which ingest plants by permeating and transferring an effective amount of the present compound, the present compound X or the composition A from roots and the like into the interior of the plant. More specific examples of the method for controlling harmful arthropods include planting hole treatment (spraying into planting holes, soil mixing after planting hole treatment), plant foot treatment (plant foot spraying, soil mixing after plant foot treatment, irrigation at plant foot, plant foot treatment at a later seeding raising stage), planting furrow treatment (planting furrow spraying, soil mixing after planting furrow treatment), planting row treatment (planting row spraying, soil mixing after planting row treatment, planting row spraying at a growing stage), planting row treatment at the time of sowing (planting row spraying at the time of sowing, soil mixing after planting row treatment at the time of sowing), broadcast treatment (overall soil surface spraying, soil mixing after broadcast treatment), side-article treatment, treatment of water surface (application to water surface, application to water surface after flooding), other soil spraying treatment (spraying of a granular formulation on leaves at a growing stage, spraying under a canopy or around a tree stem, spraying on the soil surface, mixing with surface soil, spraying into seed holes, spraying on the ground surfaces of furrows, spraying between plants), other irrigation treatment (soil irrigation, irrigation at a seedling raising stage, drug solution injection treatment, irrigation of a plant part just above the ground, drug solution drip irrigation, chemigation), seedling raising box treatment (spraying into a seedling raising box, irrigation of a seedling raising box, flooding into a seedling raising box with drug solution), seedling raising tray treatment (spraying on a seedling raising tray, irrigation of a seedling raising tray, flooding into a seedling raising tray with drug solution), seedbed treatment (spraying on a seedbed, irrigation of a seedbed, spraying on a lowland rice nursery, immersion of seedlings), seedbed soil incorporation treatment (mixing with seedbed soil, mixing with seedbed soil before sowing, spraying at sowing before covering with soils, spraying at sowing after covering with soils, mixing with covering soil, and other treatment (mixing with culture soil, plowing under, mixing with surface soil, mixing with soil at the place where raindrops fall from a canopy, treatment at a planting position, spraying of a granule formulation on flower clusters, mixing with a paste fertilizer).

As used herein, seeds or vegetative reproductive organs carrying the present compound, the present compound X or the composition A means seeds or vegetative reproductive organs in the state where the present compound, the present compound X or the composition A is adhered to a surface of the seeds or the vegetative reproductive organ. Also, the present compound, the present compound X, or the composition A which are may be adhered on the surface of the seeds or the vegetative reproductive organ may be permeated from the surface to the interior of the plant.

Also, when the composition A is adhered on the surface of the seeds or the vegetative reproductive organs, a layer consisting of single active ingredient may be multiply overlapped, a plural of the active ingredients may be mixed to form a single layer, a layer consisting of the single active ingredient and a layer consisting of the plural of the active ingredients may be multiply overlapped, or a layer consisting of the plural of the active ingredients may be multiply overlapped.

The seeds or vegetative reproductive organs used for the seed treatment may be used as itself, or any materials other than the present compound, the present compound A, or the composition A may be adhered before or after being treated with the present compound, the present compound A or the composition A.

Examples of the application to seeds (or seed treatments) include an application of the present compound X or the composition A X to seeds or vegetative reproductive organs, and specific examples thereof include spraying treatment in which a suspension of the present compound X or the composition A is sprayed onto seed surface or the vegetative reproductive organ surface in the form of mist; smearing treatment in which the present compound X or the composition A is coated a surface of seeds or the vegetative reproductive organ; a soaking treatment in which the seeds are soaked into the solution of the present compound X or the composition A for a certain time; and a method for coating the seeds or the vegetative reproductive organ with a carrier containing the present compound X or the composition A (film coating treatment, pellet coating treatment). Examples of the above-described vegetative reproductive organ include particularly seed potato.

When the composition A is applied to seeds or vegetative reproductive organs, the composition A may be also applied to seeds or vegetative reproductive organs as a single formulation, or the composition A may be applied to seeds or vegetative reproductive organs as a divided plural of formulations by a plurality of times. Examples of the method in which the composition A is applied as a divided plural of formulations by a plurality of times include, for example, a method in which the formulations comprising as an active component the present compound X only are applied, and seeds or vegetative reproductive organs are air dried, followed by applying the formulations comprising the present ingredient; and a method in which the formulations comprising as an active component the present compound X and the present ingredients are applied, and seeds or vegetative reproductive organs are air dried, followed by applying the formulations comprising the present ingredients other than the already-applied present ingredients, are included.

As used herein, seeds or vegetative reproductive organs carrying the present compound X or the composition A means seeds or vegetative reproductive organs in the state where the present compound X or the composition A is adhered to a surface of the seeds or the vegetative reproductive organ. The above-described seeds or vegetative reproductive organs carrying the present compound X or the composition A may be adhered by any other materials that are different from the present compound X or the composition A before or after being adhered the present compound X or the composition A to the seeds or vegetative reproductive organs.

Also, when the composition A is adhered in a form of layer(s) to a surface of seeds or vegetative reproductive organ, the layer(s) is/are composed of one layer or a plural of layers. Also, when a plural layers are formed, each of the layer may be composed of a layer comprising one or more active ingredients, or a combination of a layer comprising one or more active ingredients and a layer not comprising an active ingredient.

Seeds or vegetative reproductive organs carrying the present compound X or the composition A can be obtained, for example, by applying the formulations comprising the present compound X or the composition A by the above-described application method to seeds to seeds or vegetative reproductive organs.

When the present compound, the present compound A or the composition A is applied for harmful arthropods control in agricultural fields, the application dose thereof is usually within a range of 1 to 10,000 g g of the present compound or the present compound X per 10,000 m². In the case of being applied to seeds or vegetative reproductive organs, the dose of application dose thereof is usually within a range of 0.001 to 100 g of the present compound X per 1 Kg of seeds. When the present compound, the present compound X, or the composition A is formulated into an emulsifiable concentrate, a wettable powder or a flowable etc., they are usually applied by diluting them with water so as to make an effective concentration of the active ingredients 0.01 to 10,000 ppm, and the dust formulation or the granular formulation, etc., is usually applied as itself without diluting them.

Also, the resin preparation which is processed into a sheet or a string may be applied by winding a plant with a sheet or a string of the resin preparation, putting a string of the resin preparation around a crop so that the plant is surrounded by the string, or laying a sheet of the resin preparation on the soil surface near the root of a plant.

When the present compound, the present compound A or the composition A is used to control harmful arthropods that live inside a house, the application dose as an amount of the present compound or the present compound X is usually within a range from 0.01 to 1,000 mg per 1 m² of an area to be treated, in the case of using it on a planar area. In the case of using it spatially, the application dose as an amount of the present compound or the present compound X is usually within a range from 0.01 to 500 mg per 1 m³ of the space to be treated. When the present compound, the present compound X or the composition A is formulated into emulsifiable concentrates, wettable powders, flowables or the others, such formulations are usually applied after diluting it with water in such a way that a concentration of the active ingredient is within a range from 0.1 to 10,000 ppm. In the case of being formulated into oil solutions, aerosols, smoking agents, poison baits and the others, such formulations are used as itself without diluting it.

When the present compound, the present compound X or the composition is used for controlling external parasites of livestock such as cows, horses, pigs, sheep, goats and chickens and small animals such as dogs, cats, rats and mice, the composition of the present invention may be applied to the animals by a known method in the veterinary field. Specifically, when systemic control is intended, the composition of the present invention is administered to the animals as a tablet, a mixture with feed or a suppository, or by injection (including intramuscular, subcutaneous, intravenous and intraperitoneal injections). On the other hand, when non-systemic control is intended, the composition of the present invention is applied to the animals by means of spraying of the oil solution or aqueous solution, pour-on or spot-on treatment, or washing of the animal with a shampoo formulation, or by putting a collar or ear tag made of the resin formulations to the animal. In the case of being administered to an animal body, the dose of the compound of the present compound or the present compound X is usually within a range from 0.1 to 1,000 mg per 1 kg of an animal body weight.

Also, the composition of the present compound, the present compound X or the composition A may be used as an agent for controlling harmful arthropods in agricultural lands such as paddy fields, fields, turfs, and orchards. Examples of the plants to be applied include the followings.
corn, rice, wheat, barley, rye, oat, sorghum, cotton, soybean, peanut, buckwheat, beet, rapeseed, sunflower, sugar cane, tobacco,
solanaceous vegetables (for example, eggplant, tomato, pimento, pepper, or potato),
cucurbitaceous vegetables (for example, cucumber, pumpkin, zucchini, water melon, or melon),
cruciferous vegetables (for example, Japanese radish, white turnip, horseradish, kohlrabi, Chinese cabbage, cabbage, leaf mustard, broccoli, or cauliflower),
asteraceous vegetables (for example, burdock, crown daisy, artichoke, or lettuce),
liliaceous vegetables (for example, green onion, onion, garlic, or asparagus),
ammiaceous vegetables (for example, carrot, parsley, celery, or parsnip),
chenopodiaceous vegetables (for example, spinach, or Swiss chard),
lamiaceous vegetables (for example, Perilla frutescens, mint, or basil),
strawberry, sweet potato, Dioscorea japonica, colocasia,
pomaceous fruits (for example, apple, pear, Japanese pear, Chinese quince, or quince),
stone fleshy fruits (for example, peach, plum, nectarine, Prunus mume, cherry fruit, apricot, or prune),
citrus fruits (for example, Citrus unshiu, orange, lemon, lime, or grapefruit),
nuts (for example, chestnut, walnuts, hazelnuts, almond, pistachio, cashew nuts, or macadamia nuts),
   berry fruits (for example, blueberry, cranberry, blackberry or raspberry),
grape, kaki persimmon, olive, Japanese plum, banana, coffee, date palm, coconuts, tea, mulberry, ornamental foliage plants, woodland plants, lawns, pastures.

The above-mentioned plants are not particularly limited as long as they are commonly cultivated varieties. The above-mentioned plants may include plants which can be produced by a natural mating, plants which is developed by a mutation, F1 hybrid plants, and genetically modified crops. Examples of the genetically modified crops include plants which is imparted with resistance to herbicides including HPPD (that is, 4-hydroxyphenylpyruvate dioxygenase) inhibitors such as isoxaflutole; ALS (that is, acetoacetate synthase) inhibitors such as imazethapyr and thifensulfuron methyl; EPSP (that is, 5-enolpyruvoylshikimate-3-phosphate synthase) inhibitors; glutamine synthetase inhibitors; PPO (that is, protoporphyrinogen oxidase) inhibitors; bromoxynil; dicamba, and the like; plants which have become capable of synthesizing selective toxins and the like (for example, genus Bacillus such as Bacillus thuringiensis); and the plants being capable of synthesizing a gene segment that match partially an endogenous gene derived from a harmful insect and also imparting with specific insecticidal activity by inducing a gene silencing (RNAi; RNA interference) in a target harmful insect.

### EXAMPLES

Hereinafter, the present invention is explained in more detail by using Preparation examples, Reference preparation examples, and Test examples, however, the present invention should not be limited to these examples.

As used herein, “Me” represents a methyl group, “Et” represents an ethyl group, “Pr” represents a propyl group, “i-Pr” represents an isopropyl group, “c-Pr” represents a cyclopropyl group, “c-Bu” represents a cyclobutyl group, “c-Pen” represents a cyclopentyl group, “c-Hex” represents a cyclohexyl group, “Ph” represents a phenyl group, “Py2” represents a 2-pyridyl group, “Py3” represents a 3-pyridyl group, “Py4” represents a 4-pyridyl group, “Bn” represents benzyl group. “Boc” represents a tert-butoxycarbonyl group. When c-Pr, c-Bu, c-Pen, c-Hex, Ph, Py2, Py3, and Py4 have a substituent, the substituent is written with its substituted position before the symbol. For example, “1-CN-c-Pr” represents a 1-cyanocyclopropyl group, “3,4-F₂-Ph” represents a 3,4-difluorophenyl group, “4-CF₃—Py2” represents a 4-(trifluoromethyl)-2-pyridyl, and “5-OCH₂CF₂CF₃—Py2” represents a 5-(2,2,3,3,3-pentafluoropropoxy)-2-pyridyl group.

Herein, when “present compound X” is referred to, it encompasses “present compound” unless otherwise specified. Also herein, when “present compound P” is referred to, it encompasses “present compound N” unless otherwise specified.

Firstly, examples of the present compound X and process intermediate compound thereof are described.

### Reference Preparation Example 1

A mixture of 4-(trifluoromethyl)antranilic acid 3.00 g and formamide 6 mL was stirred at 140° C. for 11 hours. The resulting mixture was stood to cool to room temperature, and water was added thereto, and the mixture was filtered. The obtained solids were dried under reduced pressure to obtain the intermediate compound 1 represented by the following formula 2.90 g.

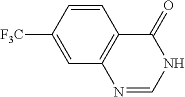 [SMILES: O=c1[nH]cnc2cc(C(F)(F)F)ccc12]

Intermediate compound 1: ¹H-NMR (CDCl₃) δ: 10.46 (1H, br s), 8.43 (1H, d), 8.14 (1H, s), 8.05 (1H, d), 7.75 (1H, dd).

### Reference Preparation Example 2

To a mixture of 4-(trifluoromethyl)antranilic acid 3.00 g and THF 22 mL was added dropwise a mixture of triphosgene 1.52 g and THF 15 mL under ice-cooling. The resulting mixture was stirred at room temperature for 4 hours. Water was added to the resulting mixture, and the mixture was extracted with ethyl acetate. The resulting organic layer was dried over anhydrous sodium sulfate, and concentrated under reduced pressure to obtain the intermediate compound 2 represented by the following formula 3.30 g.

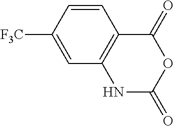 [SMILES: O=c1[nH]c2cc(C(F)(F)F)ccc2c(=O)o1]

Intermediate compound 2: ¹H-NMR (DMSO-D₆) δ: 11.98 (1H, br s), 8.10 (1H, d), 7.54 (1H, d), 7.37 (1H, s).

### Reference Preparation Example 3

To a mixture of 6-bromo-3-(ethylthio)imidazo[1,2-a]pyridin-2-amine 300 mg (prepared as described in WO/2018052136) and THF 4 mL was added dropwise a solution of potassium bis(trimethylsilyl)amide (1 mol/L THF solution) 2.2 mL at −78° C. under nitrogen atmosphere, and the mixture was stirred for 30 minutes. A mixture of the intermediate compound 2 and THF 4 mL was added to the resulting mixture, and the mixture was stirred at room temperature for 30 minutes. Saturated aqueous ammonium chloride solution was added to the resulting mixture, and the mixture was extracted with ethyl acetate. The resulting mixture was dried over anhydrous sodium sulfate, and concentrated under reduced pressure. Hexane was added to the resulting residue, and the obtained solids were filtered. The filtered substances were washed with hexane to obtain the intermediate compound 3 represented by the following formula 491 mg.

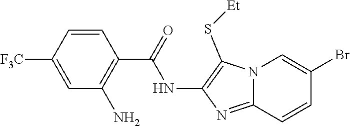 [SMILES: CCSc1c(NC(=O)c2ccc(C(F)(F)F)cc2N)nc2ccc(Br)cn12]

Intermediate compound 3-1: ¹H-NMR (CDCl₃) δ: 8.54 (1H, dd), 8.35 (1H, s), 7.67 (1H, d), 7.60 (1H, dd), 7.41 (1H, dd), 7.00 (1H, s), 6.98 (1H, d), 5.95 (2H, br s), 2.75 (2H, q), 1.27 (3H, t).

The compounds which were prepared according to the Reference Preparation Example 3 and their physical property values were shown below.

A compound represented by formula (B-1):

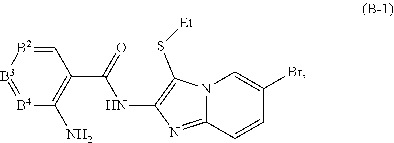

wherein a combination of B², B³ and B⁴ represents any combination indicated in [Table B¹].

TABLE B1

| Intermediate | | | |
|---|---|---|---|
| compound | B² | B³ | B⁴ |
| 3-2 | CCF₃ | CH | CH |
| 3-3 | CH | CH | CCF₃ |
| 3-4 | CH | CCF₃ | N |
| 3-5 | CH | CCl | CH |
| 3-6 | CH | CI | CH |
| 3-7 | COCF₃ | CH | CH |

Intermediate compound 3-2: ¹H-NMR (CDCl₃) δ: 8.53-8.53 (1H, m), 8.33 (1H, s), 7.80 (1H, s), 7.57 (1H, d), 7.50 (1H, dd), 7.39 (1H, dd), 6.78 (1H, d), 6.14 (2H, s), 2.74 (2H, q), 1.26 (3H, t).

Intermediate compound 3-3: ¹H-NMR (CDCl₃) δ: 8.51 (1H, dd), 8.25 (1H, br s), 7.73 (1H, d), 7.62 (1H, d), 7.57 (1H, dd), 7.38 (1H, dd), 6.77 (1H, t), 6.39 (2H, br s), 2.72 (2H, q), 1.24 (3H, t).

Intermediate compound 3-4: ¹H-NMR (CDCl₃) δ: 8.52 (1H, d), 8.35 (1H, br s), 7.98 (1H, d), 7.56 (1H, d), 7.40 (1H, dd), 7.03 (1H, d), 6.69 (2H, br s), 2.73 (2H, q), 1.24 (3H, t).

Intermediate compound 3-5: ¹H-NMR (CDCl₃) δ: 8.50 (1H, dd), 8.29 (1H, s), 7.56 (1H, dd), 7.47 (1H, d), 7.37 (1H, dd), 6.73 (1H, d), 6.68 (1H, dd), 5.87 (2H, s), 2.71 (2H, q), 1.23 (3H, t).

Intermediate compound 3-6: ¹H-NMR (CDCl₃) δ: 8.49 (1H, d), 8.28 (1H, br s), 7.56 (1H, d), 7.36 (1H, dd), 7.23 (1H, d), 7.13 (1H, d), 7.04 (1H, dd), 5.79 (2H, br s), 2.71 (2H, q), 1.22 (3H, t).

Intermediate compound 3-7: ¹H-NMR (CDCl₃) δ: 8.51 (1H, s), 8.26 (1H, s), 7.56 (1H, d), 7.39-7.36 (2H, m), 7.18 (1H, d), 6.72 (1H, d), 5.77 (2H, s), 2.73 (2H, q), 1.24 (3H, t).

### Reference Preparation Example 4

To a mixture of 6-bromo-imidazo[1,2-a]pyridin-2-amine 0.47 g (prepared as described in WO2012/173412) and THF 5 mL was added dropwise a solution of potassium bis(trimethylsilyl)amide (1 mol/L THF solution) 4.4 mL at −78° C. under nitrogen atmosphere, and the mixture was stirred for 30 minutes. A mixture of the intermediate compound 2 0.61 g and THF 5 mL was added to the resulting mixture, and the mixture was stirred at room temperature for 30 minutes. Saturated aqueous ammonium chloride solution was added to the resulting mixture, and the mixture was extracted with ethyl acetate. The resulting organic layer was dried over anhydrous sodium sulfate, and concentrated under reduced pressure. Hexane was added to the resulting residue, and the obtained solids were filtered. The filtered substances were washed with hexane to obtain the intermediate compound 4 represented by the following formula 0.66 g.

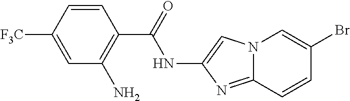 [SMILES: Nc1cc(C(F)(F)F)ccc1C(=O)Nc1cn2cc(Br)ccc2n1]

Intermediate compound 4: ¹H-NMR (DMSO-D₆) δ: 11.20 (1H, s), 8.95 (1H, dd), 8.29 (1H, s), 7.94 (1H, d), 7.46 (1H, d), 7.36 (1H, dd), 7.11 (1H, d), 6.82 (1H, d), 6.81 (2H, br s).

### Reference Preparation Example 5

A mixture of the intermediate compound 4 0.66 g and triethyl orthoformate 20 mL was stirred at 100° C. for 4 hours. Triethyl orthoformate 10 mL was added to the resulting mixture, and the mixture was stirred at 100° C. for 2 hours. The resulting mixture was stood to cool to room temperature, and concentrated under reduced pressure. The obtained solids were washed with hexane to obtain the intermediate compound 5 represented by the following formula 0.54 g.

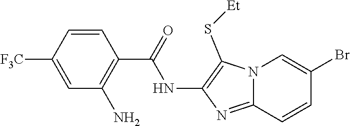 [SMILES: CCSc1c(NC(=O)c2ccc(C(F)(F)F)cc2N)nc2ccc(Br)cn12]

Intermediate compound 5: ¹H-NMR (CDCl₃) δ: 9.50 (1H, s), 8.53-8.52 (1H, m), 8.54 (1H, d), 8.36 (1H, dd), 8.10 (1H, d), 7.77 (1H, dd), 7.53 (1H, d), 7.38 (1H, dd).

### Reference Preparation Example 6

To a mixture of the intermediate compound 5 0.54 g and DMF 26 mL was added N-iodosuccinimide 0.44 g under ice-cooling, and the mixture was stirred at room temperature for 10 hours. N-iodosuccinimide 0.30 g was added to the resulting mixture under ice-cooling, and the mixture was stirred at room temperature for 6 hours. Water was added to the resulting mixture, and the obtained solids were dissolved in chloroform, and washed with saturated aqueous sodium thiosulfate solution. The resulting organic layer was dried over anhydrous sodium sulfate, and concentrated under reduced pressure to obtain the intermediate compound 6 represented by the following formula 0.32 g.

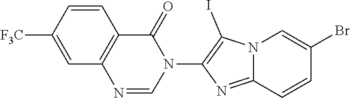 [SMILES: O=c1c2ccc(C(F)(F)F)cc2ncn1-c1nc2ccc(Br)cn2c1I]

Intermediate compound 6: ¹H-NMR (CDCl₃) δ: 8.53 (1H, d), 8.37 (1H, d), 8.26 (1H, s), 8.10-8.07 (1H, m), 7.79 (1H, dd), 7.57 (1H, d), 7.47 (1H, dd).

### Reference Preparation Example 7

To a mixture of 3-(trifluoromethylthio)aniline 5.00 g and toluene 150 mL was added N-bromosuccinimide 4.83 g at room temperature, and the mixture was stirred at room temperature for 1 hour. Water was added to the resulting mixture, and the mixture was extracted with ethyl acetate. The resulting organic layer was dried over anhydrous sodium sulfate, and concentrated udder reduced pressure. The resulting residue was subjected to a silica gel column chromatography to obtain the intermediate compound 7-1 represented by the following formula 2.99 g.

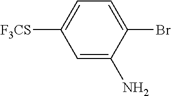

Intermediate compound 7-1: ¹H-NMR (CDCl₃) δ: 7.44 (1H, d), 7.03 (1H, d), 6.87 (1H, dd), 4.23 (2H, s).

### Reference Preparation Example 8

The intermediate compound 7-2 was obtained by using 4-(trifluoromethylthio)aniline in place of 3-(trifluoromethylthio)aniline according to the method described in the Reference Preparation Example 7.

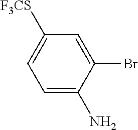

Intermediate compound 7-2: ¹H-NMR (CDCl₃) δ: 7.71 (1H, d), 7.37 (1H, dd), 6.75 (1H, d), 4.41 (2H, s).

### Reference Preparation Example 9

A mixture of the intermediate compound 7-1 2.99 g, di-tert-butyl dicarbonate 2.88 g, and THF 22 mL was stirred at 70° C. for 4 hours. Water was added to the resulting mixture, and the mixture was extracted with ethyl acetate. The resulting mixture was dried over anhydrous sodium sulfate, and concentrated under reduced pressure. The resulting residue was subjected to a silica gel column chromatography to the intermediate compound 8-1 represented by the following formula 4.64 g.

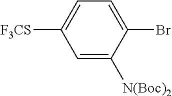

Intermediate compound 8-1: ¹H-NMR (CDCl₃) δ: 7.68 (1H, d), 7.53 (1H, d), 7.46 (1H, dd), 1.39 (18H, s).

### Reference Preparation Example 10

The intermediate compound 8-2 was obtained by using the intermediate compound 7-2 in place of the intermediate compound 7-1 according to the method described in the Reference Preparation Example 9.

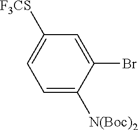

Intermediate compound 8-2: ¹H-NMR (CDCl₃) δ: 7.92 (1H, d), 7.61 (1H, dd), 7.28 (1H, d), 1.39 (18H, s).

### Reference Preparation Example 11

To a mixture of the intermediate compound 8-1 2.0 g and THF 42 ml was added dropwise butyl lithium 3 mL at −78° C. under nitrogen atmosphere, and the mixture was stirred for 15 minutes. Saturated aqueous ammonium chloride solution was added to the resulting mixture, and the mixture was extracted with ethyl acetate. The resulting organic layer was dried over anhydrous sodium sulfate, and concentrated under reduced pressure. The resulting residue was subjected to a silica gel column chromatography to obtain the intermediate compound 9-1 represented by the following formula 0.93 g.

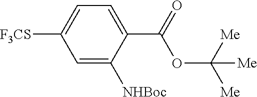

Intermediate compound 9-1: ¹H-NMR (CDCl₃) δ: 10.36 (1H, s), 8.77 (1H, s), 7.96 (1H, d), 7.22 (1H, d), 1.61 (9H, s), 1.54 (9H, s).

### Reference Preparation Example 12

The intermediate compound 9-2 was obtained by using the intermediate compound 8-2 in place of the intermediate compound 8-1 according to the method described in the Reference Preparation Example 11.

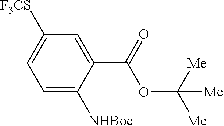

Intermediate compound 9-2: ¹H-NMR (CDCl₃) δ: 10.50 (1H, s), 8.53 (1H, d), 8.20 (1H, d), 7.71 (1H, dd), 1.62 (9H, s), 1.54 (9H, s).

### Reference Preparation Example 13

A mixture of the intermediate compound 9-1 0.93 g, trifluoroacetic acid 6 mL, and chloroform 23 ml was stirred at room temperature for 20 hours. The resulting mixture was concentrated, and the obtained solids were washed with hexane to obtain the intermediate compound 10-1 represented by the following formula 0.57 g.

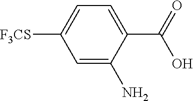

Intermediate compound 10-1: ¹H-NMR (CDCl₃) δ: 7.93 (1H, d), 6.96 (1H, s), 6.89 (1H, d).

### Reference Preparation Example 14

The intermediate compound 10-2 was obtained by using the intermediate compound 9-2 in place of the intermediate compound 9-1 according to the method described in the Reference Preparation Example 13.

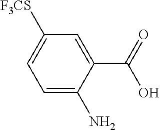

Intermediate compound 10-2: ¹H-NMR (CDCl₃) δ: 8.23 (1H, d), 7.53 (1H, dd), 6.70 (1H, d).

### Reference Preparation Example 15

To a mixture of 2-fluoro-4-iodobenzoic acid 3.00 g and chloroform 28 mL were added oxalyl chloride 2.2 mL and DMF 0.1 ml successively at room temperature, and the mixture was stirred at room temperature for 1 hour. The resulting mixture was concentrated and the mixture was dissolved in THF 28 mL and to the resulting mixture were added dropwise benzyl alcohol 1.8 mL and triethylamine 4.7 mL successively under ice-cooling, and the mixture was stirred at room temperature for 1 hour. Saturated aqueous sodium bicarbonate solution was added to the resulting mixture, and the mixture was extracted with ethyl acetate. The resulting mixture was dried over anhydrous sodium sulfate, and concentrated under reduced pressure. The resulting residue was subjected to a silica gel column chromatography to obtain the intermediate compound 11 represented by the following formula 3.78 g.

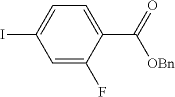 [SMILES: O=C(OCc1ccccc1)c1ccc(I)cc1F]

Intermediate compound 11: ¹H-NMR (CDCl₃) δ: 7.68-7.64 (1H, m), 7.57-7.54 (2H, m), 7.45-7.44 (2H, m), 7.41-7.33 (3H, m), 5.37 (2H, s).

### Reference Preparation Example 16

A mixture of the intermediate compound 11 2.45 g, potassium fluoride 0.76 g, copper(I) iodide 3.14 g, trimethyl(pentafluoroethyl)silane 2.4 mL and DMF 15 mL was stirred at 80° C. under microwave irradiation for 6 hours. Saturated aqueous ammonium solution and MTBE were added successively to the resulting mixture, and the mixture was filtered through Celite (registered trademark). The resulting filtrates were extracted with MTBE. The resulting organic layer was dried over anhydrous sodium sulfate, and concentrated under reduced pressure. The resulting residue was subjected to a silica gel column chromatography to obtain the intermediate compound 12 represented by the following formula 1.97 g.

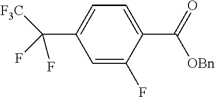 [SMILES: O=C(OCc1ccccc1)c1ccc(C(F)(F)C(F)(F)F)cc1F]

Intermediate compound 12: ¹H-NMR (CDCl₃) δ: 8.11-8.09 (1H, m), 7.47-7.33 (7H, m), 5.39 (2H, s).

### Reference Preparation Example 17

To a mixture of the intermediate compound 12 0.80 g and DMSO 5 ml was added sodium azide 194 mg at room temperature, and the mixture was stirred at 80° C. for 1 hour. Sodium azide 75 mg was added to the resulting mixture at room temperature, and the mixture was stirred at 80° C. for 3 hours. The resulting mixture was cooled under ice-cooling, and water was added thereto, and the mixture was extracted with MTBE. The resulting organic layer was dried over anhydrous sodium sulfate, and concentrated under reduced pressure. The resulting residue was subjected to a silica gel column chromatography to obtain the intermediate compound 13 represented by the following formula 13 0.85 g.

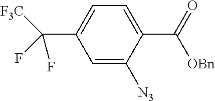 [SMILES: [N-]=[N+]=Nc1cc(C(F)(F)C(F)(F)F)ccc1C(=O)OCc1ccccc1]

Intermediate compound 13: ¹H-NMR (CDCl₃) δ: 7.98 (1H, d), 7.47-7.31 (7H, m), 5.38 (2H, s).

### Reference Preparation Example 18

A mixture of the intermediate compound 13 0.85 g, 10% palladium carbon 0.21 g, and methanol 5 mL was stirred for 8 hours under hydrogen atmosphere. The resulting mixture was filtered through Celite (registered trademark), and the filtrates were concentrated under reduced pressure to obtain the intermediate compound 14 represented by the following formula 0.64 g.

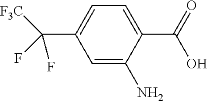 [SMILES: Nc1cc(C(F)(F)C(F)(F)F)ccc1C(=O)O]

Intermediate compound 14: ¹H-NMR (CDCl₃) δ: 8.01 (1H, d), 6.89 (1H, s), 6.85 (1H, d).

### Reference Preparation Example 19

The intermediate compound 15 was obtained by using 2-amino-5-iodobenzoic acid in place of the intermediate compound 7-1 according to the method described in the Reference Preparation Example 9.

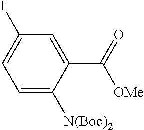

Intermediate compound 15: ¹H-NMR (CDCl₃) δ: 8.32 (1H, d), 7.84 (1H, dd), 6.93 (1H, d), 3.87 (3H, s), 1.38 (18H, s).

### Reference Preparation Example 20

The intermediate compound 16 was obtained by using the intermediate compound 15 in place of the intermediate compound 11 according to the method described in the Reference Preparation Example 16.

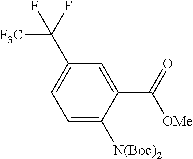

Intermediate compound 16: ¹H-NMR (CDCl₃) δ: 8.25 (1H, s), 7.76 (1H, d), 7.37 (1H, d), 3.90 (3H, s), 1.38 (18H, s).

### Reference Preparation Example 2 1

To a mixture of the intermediate compound 16 2.44 g and ethanol 13 ml was added dropwise 15% aqueous sodium hydroxide solution under ice-cooling. The resulting mixture was stirred at room temperature for 6 hours, and concentrated under reduced pressure, and neutralized with 2N hydrochloric acid. The resulting mixture was extracted with ethyl acetate, and the resulting organic layer was dried over anhydrous sodium sulfate, and concentrated under reduced pressure to obtain the intermediate compound 17 represented by the following formula 1.80 g.

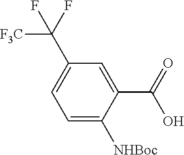 [SMILES: CC(C)(C)OC(=O)Nc1ccc(C(F)(F)C(F)(F)F)cc1C(=O)O]

Intermediate compound 17: ¹H-NMR (DMSO-D₆) δ: 8.13 (1H, s), 7.94-7.93 (1H, m), 7.62-7.61 (1H, m), 7.53 (1H, d), 1.48 (9H, s).

### Reference Preparation Example 22

The intermediate compound 18 was obtained by using the intermediate compound 17 in place of the intermediate compound 9-1 according to the method described in the Reference Preparation Example 13.

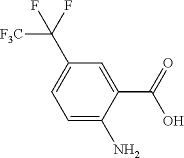 [SMILES: Nc1ccc(C(F)(F)C(F)(F)F)cc1C(=O)O]

Intermediate compound 18: ¹H-NMR (DMSO-D₆) δ: 7.91 (1H, d), 7.45 (1H, dd), 6.92 (1H, d).

### Reference Preparation Example 23

To a mixture of ethyl imidazo[1,2-a]pyridine-2-carboxylate 5.66 g and DMF 40 mL was added N-chlorosuccinimide 4.37 g under ice-cooling, and the mixture was stirred at 60° C. for 6 hours. Water was added to the resulting mixture, and the precipitated out solids were filtered. The obtained solids were washed with water, and dried under reduced pressure to obtain the intermediate compound 19-1 represented by the following formula 6.60 g.

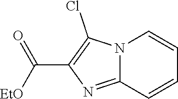 [SMILES: CCOC(=O)c1nc2ccccn2c1Cl]

Intermediate compound 19-1: ¹H-NMR (CDCl₃) δ: 8.16-8.15 (1H, m), 7.71-7.69 (1H, m), 7.36-7.31 (1H, m), 7.05-7.01 (1H, m), 4.50 (2H, q), 1.47 (3H, t).

The compounds which were prepared according to the Reference Preparation Example 23 and their physical property values were shown below.

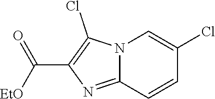 [SMILES: CCOC(=O)c1nc2ccc(Cl)cn2c1Cl]

Intermediate compound 19-2: ¹H-NMR (CDCl₃) δ: 8.19 (1H, d), 7.65 (1H, d), 7.29 (1H, dd), 4.49 (2H, q), 1.46 (3H, t).

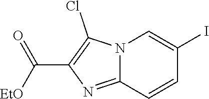 [SMILES: CCOC(=O)c1nc2ccc(I)cn2c1Cl]

Intermediate compound 19-3: ¹H-NMR (CDCl₃) δ: 8.41 (1H, dd), 7.51-7.45 (2H, m), 4.49 (2H, q), 1.46 (3H, t).

C1

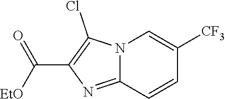 [SMILES: CCOC(=O)c1nc2ccc(C(F)(F)F)cn2c1Cl]

Intermediate compound 19-4: ¹H-NMR (CDCl₃) δ: 8.52 (1H, dd), 7.82 (1H, dd), 7.47 (1H, dd), 4.52 (2H, q), 1.48 (3H, t).

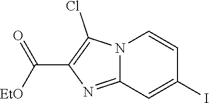 [SMILES: CCOC(=O)c1nc2cc(I)ccn2c1Cl]

Intermediate compound 19-5: ¹H-NMR (CDCl₃) δ: 8.10 (1H, dd), 7.89 (1H, dd), 7.25 (1H, dd), 4.49 (2H, q), 1.46 (3H, t).

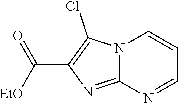 [SMILES: CCOC(=O)c1nc2ncccn2c1Cl]

Intermediate compound 19-6: ¹H-NMR (CDCl₃) δ: 8.74 (1H, dd), 8.47 (1H, dd), 7.10 (1H, dd), 4.51 (2H, q), 1.47 (3H, t).

### Reference Preparation Example 24

To a mixture of the intermediate compound 19-1 6.60 g, cesium carbonate 24.2 g and DMF 45 ml was added ethanethiol 2.4 mL at room temperature, and the mixture was stirred for 4 hours. Water was added to the resulting mixture, and the precipitated out solids were filtered. The obtained solids were washed with water and dried under reduced pressure to obtain the intermediate compound 20-1 represented by the following formula 6.76 g.

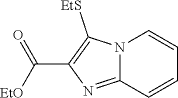

Intermediate compound 20-1: ¹H-NMR (CDCl₃) δ: 8.57-8.55 (1H, m), 7.73-7.70 (1H, m), 7.36-7.32 (1H, m), 7.01-6.98 (1H, m), 4.51 (2H, q), 2.94 (2H, q), 1.48 (3H, t), 1.19 (3H, t).

The compounds which were prepared according to the Reference Preparation Example 24 and their physical property values were shown below.

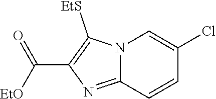

Intermediate compound 20-2: ¹H-NMR (CDCl₃) δ: 8.59 (1H, s), 7.66 (1H, d), 7.30 (1H, d), 4.50 (2H, q), 2.95 (2H, q), 1.47 (3H, t), 1.21 (3H, t).

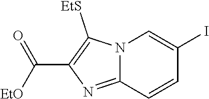

Intermediate compound 20-3: ¹H-NMR (CDCl₃) δ: 8.80 (1H, dd), 7.52-7.48 (2H, m), 4.50 (2H, q), 2.95 (2H, q), 1.47 (3H, t), 1.21 (3H, t).

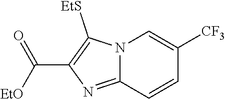

Intermediate compound 20-4: ¹H-NMR (CDCl₃) δ: 8.92 (1H, s), 7.82 (1H, d), 7.47 (1H, d), 4.52 (2H, q), 2.99 (2H, q), 1.48 (3H, t), 1.22 (3H, t).

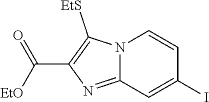

Intermediate compound 20-5: ¹H-NMR (CDCl₃) δ: 8.29 (1H, dd), 8.12 (1H, dd), 7.22 (1H, dd), 4.50 (2H, q), 2.93 (2H, q), 1.47 (3H, t), 1.18 (3H, t).

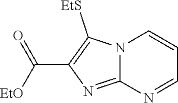

Intermediate compound 20-6: ¹H-NMR (CDCl₃) δ: 8.84 (1H, dd), 8.73 (1H, dd), 7.06 (1H, dd), 4.51 (2H, q), 2.97 (2H, q), 1.47 (3H, t), 1.19 (3H, t).

### Reference Preparation Example 25

The intermediate compound 21-1 was obtained by using the intermediate compound 20-1 in place of the intermediate compound 16 according to the method described in the Reference Preparation Example 21.

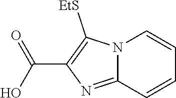

Intermediate compound 21-1: ¹H-NMR (DMSO-D₆) δ: 8.67-8.65 (1H, m), 7.71-7.69 (1H, m), 7.49-7.45 (1H, m), 7.18-7.14 (1H, m), 2.87 (2H, q), 1.05 (3H, t).

The compounds which were prepared according to the Reference Preparation Example 25 and their physical property values were shown below.

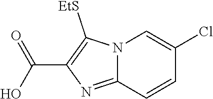

Intermediate compound 21-2: ¹H-NMR (CDCl₃) δ: 8.74 (1H, s), 7.76 (1H, d), 7.52 (1H, d), 2.89 (2H, q), 1.07 (3H, t).

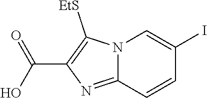

Intermediate compound 21-3: ¹H-NMR (DMSO-D₆) δ: 8.77 (1H, dd), 7.61 (1H, dd), 7.50 (1H, dd), 2.86 (2H, q), 1.03 (3H, t).

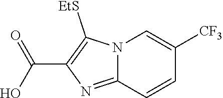

Intermediate compound 21-4: ¹H-NMR (DMSO-D₆) δ: 8.89 (1H, s), 7.82 (1H, d), 7.62 (1H, d), 2.92 (2H, q), 1.03 (3H, t).

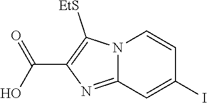

Intermediate compound 21-5: ¹H-NMR (DMSO-D₆) δ: 8.40 (1H, dd), 8.15-8.13 (1H, m), 7.34 (1H, dd), 2.85 (2H, q), 1.01 (3H, t).

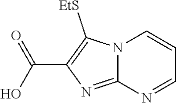

Intermediate compound 21-6: ¹H-NMR (DMSO-D₆) δ: 9.46 (1H, dd), 8.66 (1H, dd), 7.26 (1H, dd), 3.23 (2H, q), 1.37 (3H, t).

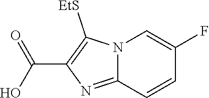

Intermediate compound 44: ¹H-NMR (CDCl₃) δ: 8.51-8.50 (1H, m), 8.18-8.15 (1H, m), 7.39-7.33 (1H, m), 3.01 (2H, q), 1.23 (3H, t).

### Reference Preparation Example 26

To a mixture of the intermediate compound 21-1 5.45 g, triethylamine 8.5 mL, and tert-butyl alcohol 50 mL was added diphenyl phosphoryladize 9.2 mL, and the mixture was stirred at 90° C. for 5 hours. The resulting mixture was concentrated, and saturated aqueous sodium bicarbonate solution was added thereto, and the mixture was extracted with ethyl acetate. The resulting organic layer was dried over anhydrous sodium sulfate, and concentrated under reduced pressure. The resulting residue was subjected to a silica gel column chromatography to obtain the intermediate compound 22-1 represented by the following formula 4.96 g.

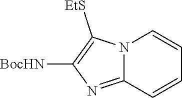

Intermediate compound 22-1: ¹H-NMR (CDCl₃) δ: 8.34-8.32 (1H, m), 7.63-7.61 (1H, m), 7.25-7.23 (1H, m), 7.00 (1H, s), 6.92-6.88 (1H, m), 2.64 (2H, q), 1.54 (9H, s), 1.19 (3H, t).

The compounds which were prepared according to the Reference Preparation Example 26 and their physical property values were shown below.

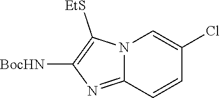

Intermediate compound 22-2: ¹H-NMR (CDCl₃) δ: 8.34 (1H, dd), 7.56 (1H, dd), 7.20 (1H, dd), 7.02 (1H, s), 2.66 (2H, q), 1.55 (9H, s), 1.21 (3H, t).

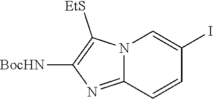

Intermediate compound 22-3: ¹H-NMR (CDCl₃) δ: 8.54 (1H, dd), 7.44-7.39 (2H, m), 7.01 (1H, s), 2.65 (2H, q), 1.55 (9H, s), 1.20 (3H, t).

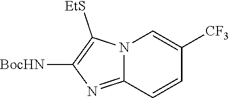

Intermediate compound 22-4: ¹H-NMR (CDCl₃) δ: 8.67 (1H, s), 7.72 (1H, d), 7.41 (1H, d), 7.08 (1H, s), 2.69 (2H, q), 1.57 (9H, s), 1.22 (3H, t).

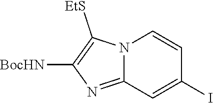

Intermediate compound 22-5: ¹H-NMR (CDCl₃) δ: 8.07 (1H, d), 8.01 (1H, s), 7.15 (1H, d), 6.99 (1H, s), 2.64 (2H, q), 1.58 (9H, s), 1.19 (3H, t).

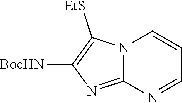

Intermediate compound 22-6: ¹H-NMR (CDCl₃) δ: 8.50 (1H, dd), 8.15 (1H, dd), 6.89 (1H, dd), 6.30 (1H, s), 3.22 (2H, q), 1.53 (9H, s), 1.36 (3H, t).

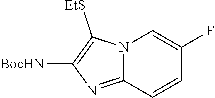

Intermediate compound 22-7: ¹H-NMR (CDCl₃) δ: 8.26-8.25 (1H, m), 7.60-7.58 (1H, m), 7.16-7.14 (1H, m), 7.00 (1H, s), 2.65 (2H, q), 1.55 (9H, s), 1.20 (3H, t).

### Reference Preparation Example 27

A mixture of the intermediate compound 22-1 4.96 g and 4 mol/L hydrogen chloride in 1,4-dioxane solution 40 mL was stirred at room temperature for 10 hours. The resulting mixture was concentrated, and water was added thereto, and the mixture was neutralized with 10 N aqueous sodium hydroxide solution. The resulting mixture was extracted with chloroform, and the resulting organic layer was dried over anhydrous sodium sulfate, and concentrated under reduced pressure to obtain the intermediate compound 23-1 represented by the following formula 3.20 g.

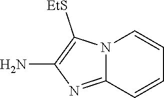

Intermediate compound 23-1: ¹H-NMR (CDCl₃) δ: 8.25-8.23 (1H, m), 7.35-7.33 (1H, m), 7.19-7.15 (1H, m), 6.82-6.79 (1H, m), 4.30 (2H, s), 2.60 (2H, q), 1.20 (3H, t).

The compounds which were prepared according to the Reference Preparation Example 27 and their physical property values were shown below.

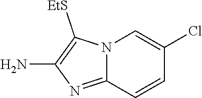

Intermediate compound 23-2: ¹H-NMR (DMSO-D₆) δ: 8.34 (1H, dd), 7.26 (1H, dd), 7.20 (1H, dd), 5.57 (2H, s), 2.61 (2H, q), 1.09 (3H, t).

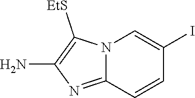

Intermediate compound 23-3: ¹H-NMR (CDCl₃) δ: 8.43 (1H, dd), 7.34 (1H, dd), 7.13 (1H, dd), 4.31 (2H, s), 2.61 (2H, q), 1.21 (3H, t).

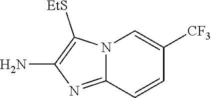

Intermediate compound 23-4: ¹H-NMR (CDCl₃) δ: 8.56 (1H, dd), 7.41 (1H, dd), 7.32 (1H, dd), 4.43 (2H, s), 2.64 (2H, q), 1.23 (3H, t).

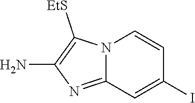

Intermediate compound 23-5: ¹H-NMR (CDCl₃) δ: 7.97 (1H, dd), 7.71 (1H, dd), 7.05 (1H, dd), 4.32 (2H, s), 2.59 (2H, q), 1.19 (3H, t).

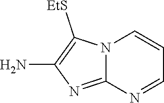

Intermediate compound 23-6: ¹H-NMR (DMSO-D₆) δ: 8.44 (1H, dd), 8.27 (1H, dd), 6.93 (1H, dd), 5.23 (2H, s), 2.87 (2H, q), 1.18 (3H, t).

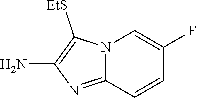

Intermediate compound 23-7: ¹H-NMR (CDCl₃) δ: 8.17-8.17 (1H, m), 7.31-7.27 (1H, m), 7.08-7.04 (1H, m), 4.29 (2H, s), 2.61 (2H, q), 1.22 (3H, t).

### Reference Preparation Example 28

A mixture of 6-bromo-3-(ethylthio)imidazo[1,2-b]pyridine-2-amine 1.00 g, which was prepared according to the method described in WO 2018/052136, trimethylboroxine 507 mg, [1,1′-bis(diphenylphosphino)ferrocene]dichloropalladium(II) dichloromethane complex 340 mg, potassium carbonate 1.52 g, and 1,4-dioxane 36 ml was stirred at 100° C. for 8 hours. The resulting mixture was stood to cool to room temperature, and water was then added thereto, and the mixture was extracted with ethyl acetate. The resulting organic layer was dried over anhydrous sodium sulfate, and concentrated under reduced pressure. The resulting residue was subjected to a silica gel column chromatography to obtain the intermediate compound 24 represented by the following formula 0.19 g.

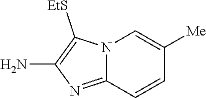

Intermediate compound 24: ¹H-NMR (CDCl₃) δ: 8.03-8.02 (1H, m), 7.26-7.23 (1H, m), 7.02 (1H, dd), 4.22 (2H, s), 2.59 (2H, q), 2.34 (3H, s), 1.21 (3H, t).

### Reference Preparation Example 29

To a mixture of 2-amino-5-bromopyrimidine 50.0 g and pyridine 280 mL was added dropwise a mixture of p-toluenesulfonyl chloride 164 g and pyridine 280 mL over 40 minutes at room temperature. The resulting mixture was stirred at 80° C. for 15 hours, and stood to cool to room temperature, and water was added thereto. The precipitated out solids were filtered. The obtained solids were washed with water and ethanol successively, and dried under reduced pressure to obtain the intermediate compound 25-1 represented by the following formula 70.6 g.

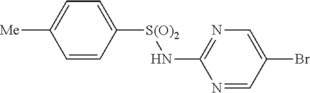

Intermediate compound 25-1: ¹H-NMR (CDCl₃) δ: 9.69 (1H, s), 8.58 (2H, s), 7.99 (2H, d), 7.31 (2H, d), 2.42 (3H, s).

### Reference Preparation Example 30

The intermediate compound 25-2 was obtained by using 2-amino-5-iodopyrimidine in place of 2-amino-5-bromopyrimidine according to the method described in Reference Preparation Example 29.

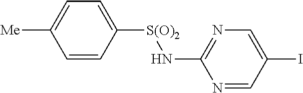

Intermediate compound 25-2: ¹H-NMR (CDCl₃) δ: 9.56 (1H, s), 8.68 (2H, s), 7.99 (2H, d), 7.31 (2H, d), 2.42 (3H, s).

### Reference Preparation Example 31

To a mixture of the intermediate compound 25-1 60.8 g and DMF 370 mL was added dropwise diisopropylethylamine 39 mL at room temperature, and the mixture was stirred for 50 minutes. 2-Iodoacetamide 41.1 g was added to the resulting mixture, and the mixture was stirred at room temperature for 8.5 hours. Water was added to the resulting mixture, and the precipitated out solids were filtered. The obtained solids were washed with water and dried under reduced pressure.

The obtained solids were dissolved in chloroform 500 mL, and trifluoroacetic anhydride 500 ml was added thereto, and the mixture was stirred at 40° C. for 6 hours. The resulting mixture was concentrated under reduced pressure, and ethyl acetate and saturated aqueous sodium bicarbonate solution were added successively to the resulting residue, and the mixture was extracted with ethyl acetate. The resulting organic layer was dried over anhydrous sodium sulfate, and concentrated under reduced pressure to obtain the intermediate compound 26-1 represented by the following formula 16.4 g.

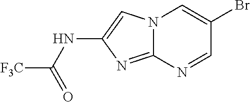 [SMILES: O=C(Nc1cn2cc(Br)cnc2n1)C(F)(F)F]

Intermediate compound 26-1: ¹H-NMR (DMSO-D₆) δ: 12.76 (1H, s), 9.33 (1H, d), 8.56 (1H, d), 8.17 (1H, s).

### Reference Preparation Example 32

The intermediate compound 26-2 was obtained by using the intermediate compound 25-2 in place of the intermediate compound 25-1 according to the method described in Reference Preparation Example 31.

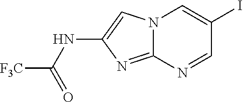 [SMILES: O=C(Nc1cn2cc(I)cnc2n1)C(F)(F)F]

Intermediate compound 26-2: ¹H-NMR (DMSO-D₆) δ: 12.73 (1H, s), 9.35 (1H, d), 8.60 (1H, d), 8.13 (1H, s).

### Reference Preparation Example 33

To a mixture of the intermediate compound 26-1 8.20 g and NMP 88 mL were added diethyl disulfide 6.5 ml and iodine 12.1 g successively at room temperature, and the mixture was stirred at 110° C. for 2.5 hours. The resulting mixture was stood to cool to room temperature, and ethyl acetate, water and saturated aqueous sodium thiosulfate solution were added successively thereto, and the mixture was extracted with ethyl acetate. The resulting organic layer was dried over anhydrous sodium sulfate, and concentrated under reduced pressure. The resulting residue was subjected to a silica gel column chromatography to obtain the intermediate compound 27-1 represented by the following formula 3.84 g.

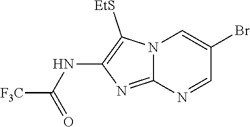

Intermediate compound 27-1: ¹H-NMR (CDCl₃) δ: 8.81 (1H, d), 8.63 (1H, d), 2.82 (2H, q), 1.25 (3H, t).

### Reference Preparation Example 34

The intermediate compound 27-2 was obtained by using the intermediate compound 26-2 in place of the intermediate compound 26-1 according to the method described in the Reference Preparation Example 33.

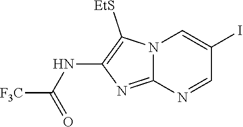

Intermediate compound 27-2: ¹H-NMR (CDCl₃) δ: 8.90 (1H, d), 8.69 (1H, d), 2.84-2.81 (2H, m), 1.25 (3H, t).

### Reference Preparation Example 35

A mixture of the intermediate compound 27-1 3.84 g, potassium carbonate 7.19 g, water 100 mL and methanol 200 mL was stirred at 80° C. for 1 hour. The resulting mixture was concentrated, and water was added thereto, and the precipitated out solids were filtered. The obtained solids were died under reduced pressure to obtain the intermediate compound 28-1 represented by the following formula 2.00 g.

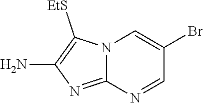

Intermediate compound 2-81: ¹H-NMR (CDCl₃) δ: 8.52 (1H, d), 8.34 (1H, d), 4.60 (2H, s), 2.63 (2H, q), 1.23 (3H, t).

### Reference Preparation Example 36

The intermediate compound 28-2 was obtained by using the intermediate compound 27-2 in place of the intermediate compound 27-1 according to the method described in the Reference Preparation Example 35.

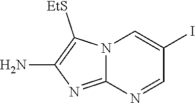

Intermediate compound 28-2: ¹H-NMR (DMSO-D₆) δ: 8.75 (1H, d), 8.33 (1H, d), 6.02 (2H, s), 2.64 (2H, d), 1.09 (3H, t).

### Reference Preparation Example 37

To a mixture of chloroacetic acid 9.49 g and water 15 ml was added triethylamine 16.7 ml at 0° C. over 30 minutes. 2-Amino-5-(trifluoromethyl)pyridine 16.1 g was added to the resulting mixture and the mixture was stirred under reflux for 2 hours. The resulting mixture was stood to cool to room temperature, and the precipitated out solids were filtered. The obtained solids were washed with water, and dried reduced pressure to obtain the intermediate compound 29-1 represented by the following formula 11.0 g.

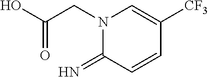 [SMILES: N=c1ccc(C(F)(F)F)cn1CC(=O)O]

Intermediate compound 29-1: LCMS: 219 [M−H]⁻, RT=0.42 minutes

The compound which was prepared according to the Reference Preparation Example 37 and its physical property value was shown below.

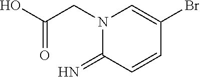 [SMILES: N=c1ccc(Br)cn1CC(=O)O]

Intermediate compound 29-2: LCMS: 229 [M−H]⁻, RT=0.34 minutes

### Reference Preparation Example 38

A mixture of the crude product of the intermediate compound 29-1 13.21 g, which was prepared according to the Reference Preparation Example 37, phosphorus oxychloride 18 mL, and toluene 150 mL was stirred under reflux for 6 hours. The resulting mixture was added dropwise to aqueous solution of sodium hydroxide, and the mixture was extracted with toluene. The resulting organic layer was dried over anhydrous sodium sulfate, and concentrated under reduced pressure to obtain the intermediate compound 30-1 represented by the following formula 13.2 g.

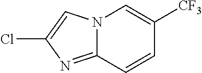 [SMILES: FC(F)(F)c1ccc2nc(Cl)cn2c1]

Intermediate compound 30-1: ¹H-NMR (CDCl₃) δ: 8.44 (1H, s), 7.65 (1H, d), 7.62 (1H, s), 7.38 (1H, d).

### Reference Preparation Example 39

To a mixture of the intermediate compound 30-1 15.44 g and DMF 75 mL was added N-iodosuccinimide 17.32 g under ice-cooling, and the mixture was stirred at 70° C. for 5 hours. An aqueous solution of sodium thiosulfate was added to the resulting mixture, and the precipitated out solids were collected by filtration. The obtained solids were washed with water and dried under reduced pressure to obtain the intermediate compound 31-1 represented by the following formula 18.0 g.

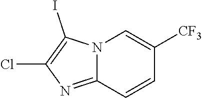 [SMILES: FC(F)(F)c1ccc2nc(Cl)c(I)n2c1]

Intermediate compound 31-1: ¹H-NMR (CDCl₃) δ: 8.41 (1H, s), 7.65 (1H, d), 7.44 (1H, d).

The compound which was prepared according to the Reference Preparation Example 39 and its physical property value was shown below.

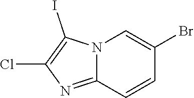 [SMILES: Clc1nc2ccc(Br)cn2c1I]

Intermediate compound 31-2: ¹H-NMR (CDCl₃) δ: 8.20 (1H, s), 7.43 (1H, d), 7.35 (1H, d).

### Reference Preparation Example 40

A mixture of the intermediate compound 31-1 18.0 g, 1,4-dioxane 140 mL, tris(dibenzylideneacetone)dipalladium(0) 2.38 g, Xantphos 3.01 g, diisopropylethylamine 27.2 mL and ethanethiol 3.75 mL was stirred under reflux for 3 hours. The resulting mixture was cooled to room temperature, and concentrated under reduced pressure. The residue was subjected to a silica gel column chromatography to obtain the intermediate compound 32-1 represented by the following formula 13.39 g.

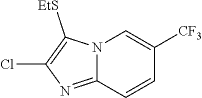

Intermediate compound 32-1: ¹H-NMR (CDCl₃) δ: 8.74 (1H, s), 7.67 (1H, d), 7.48 (1H, d), 2.78 (2H, d), 1.24 (3H, t).

The compounds which were prepared according to the Reference Preparation Example 40 and their physical property values were shown below.

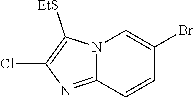

Intermediate compound 32-2: ¹H-NMR (CDCl₃) δ: 8.51 (1H, s), 7.46 (1H, d), 7.38 (1H, d), 2.73 (2H, d), 1.23 (3H, t).

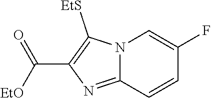

Intermediate compound 32-3: ¹H-NMR (CDCl₃) δ: 8.49-8.48 (1H, m), 7.70 (1H, dd), 7.29-7.24 (1H, m), 4.50 (2H, q), 2.95 (2H, q), 1.47 (3H, t), 1.20 (3H, t).

### Reference Preparation Example 41

To a mixture of the intermediate compound 32-2 2.66 g and chloroform 10 mL was added mCPBA (purity 70%, 30% water content) 5.16 g under ice-cooling, and the mixture was stirred at room temperature for 2 hours. Saturated aqueous solution of sodium hydrogen carbonate and aqueous solution of sodium thiosulfate were added successively to the resulting mixture, and the mixture was extracted with ethyl acetate. The resulting organic layer was dried over anhydrous sodium sulfate, and concentrated under reduced pressure. The resulting residue was subjected to a silica gel column chromatography to obtain the intermediate compound 33-1 represented by the following formula 1.79 g.

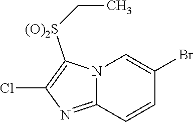

Intermediate compound 33-1: ¹H-NMR (CDCl₃) δ: 9.15 (1H, s), 7.60 (1H, d), 7.57 (1H, d), 3.36 (2H, q), 1.36 (3H, t).

The compound which was prepared according to the Reference Preparation Example 41 and its physical property value was shown below.

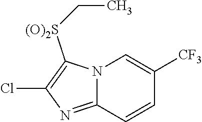

Intermediate compound 33-2: ¹H-NMR (CDCl₃) δ: 9.39 (1H, s), 7.81 (1H, d), 7.67 (1H, d), 3.39 (2H, q), 1.37 (3H, t).

### Reference Preparation Example 42

To a mixture of the intermediate compound 33-1 324 mg, trans-N,N′-dimethylcyclohexane-1,2-diamine 0.32 mL, sodium iodide 225 mg, copper(I) iodide 190 mg, and toluene 4 ml was stirred at 120° C. for 21 hours. The resulting mixture was cooled to room temperature, and then filtered. Water was added to the resulting filtrate, and the mixture was extracted with chloroform. The resulting organic layer was dried over anhydrous sodium sulfate, and concentrated under reduced pressure. The resulting residue was subjected to a silica gel column chromatography to obtain the intermediate compound 34 represented by the following formula 70 mg.

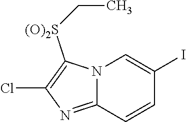

Intermediate compound 34: ¹H-NMR (CDCl₃) δ: 9.23 (1H, s), 7.70 (1H, d), 7.46 (1H, d), 3.35 (2H, m), 1.35 (3H, t).

### Reference Preparation Example 43

A mixture of the intermediate compound 33-2 936 mg, cesium fluoride 4.56 g and DMSO 10 mL was stirred at 95° C. for 2 hours. The resulting mixture was cooled to room temperature, and ethyl acetate and water were then added successively to the resulting mixture, and the mixture was filtered through Celite (Registered Trademark). The resulting filtrates were separated with a separatory funnel, and the resulting organic layer was dried over anhydrous sodium sulfate, and concentrated under reduced pressure. The resulting residue was subjected to silica gel column chromatography to obtain a crude product of the intermediate compound 35-1 represented by the following formula (containing 22% of the intermediate compound 27) 330 mg.

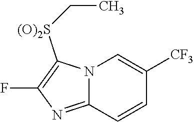

Intermediate compound 35-1: LCMS: 297 [M+H]⁺, RT=1.76 minutes

The compounds which were prepared according to the Reference Preparation Example 43 and their physical property values were shown below.

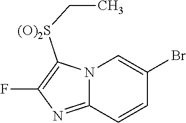

Intermediate compound 35-2: LCMS: 307 [M+H]⁺, RT=1.64 minutes

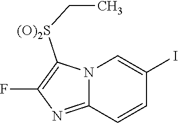

Intermediate compound 35-3: LCMS: 355 [M+H]⁺, RT=1.72 minutes

### Reference Preparation Example 44

To a mixture of 2-aminobenzaldehyde 12.1 g and DMF 100 mL was added N-iodosuccinimide 22.5 g portion wise at room temperature, and the mixture was stirred at room temperature for 4 hours. Aqueous solution of sodium bicarbonate and aqueous solution of sodium thiosulfate were added to the resulting mixture successively under ice-cooling, and the precipitated out solids were filtered. The obtained solids were washed with water, and dried under reduced pressure to obtain the intermediate compound 36-1 represented by the following formula 23.6 g.

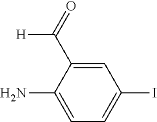

Intermediate compound 36-1: ¹H-NMR (CDCl₃) 9.79 (1H, s), 7.75 (1H, d), 7.52 (1H, dd), 6.47 (1H, d), 6.15 (2H, s).

### Reference Preparation Example 45

The intermediate compound 36-2 was obtained by using N-bromosuccinimide in place of N-iodosuccinimide according to the method described in the Reference Preparation Example 44.

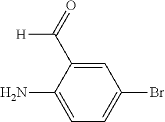

Intermediate compound 36-2: ¹H-NMR (CDCl₃) δ: 9.80 (1H, s), 7.58 (1H, d), 7.37 (1H, dd), 6.57 (1H, d), 6.15 (2H, s).

### Reference Preparation Example 46

To a mixture of 2-(ethansulfonyl)acetic acid 15.23 g, DMF 0.1 mL and chloroform 60 mL was added dropwise oxalyl chloride 12.9 mL under ice-cooling. The resulting mixture was stirred at room temperature for 1 hour, and then concentrated under reduced pressure. The resulting residue was dissolved in acetonitrile 20 mL, and the resulting mixture was added dropwise to a mixture of the intermediate compound 36-1 23.6 g and acetonitrile 100 mL under ice-cooling. The resulting mixture was stirred at room temperature for 4 hours, and concentrated under reduced pressure. The resulting residue was dissolved in acetonitrile 100 mL, and triethylamine 19.9 mL was added dropwise thereto under ice-cooling, and the mixture was stirred at room temperature for 1 hour. Water was added to the resulting mixture under ice-cooling. The precipitated out solids were filtered, and the obtained solids were washed with water. The obtained solids were washed with a solution of MTBE:ethyl acetate=1:1, and concentrated under reduced pressure to obtain the intermediate compound 37-1 represented by the following formula 13.12 g.

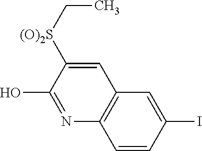

Intermediate compound 37-1: ¹H-NMR (DMSO-D₆) δ: 8.69 (1H, s), 8.43 (1H, d), 7.97 (1H, dd), 7.20 (1H, d), 3.51 (2H, q), 1.15 (3H, t).

### Reference Preparation Example 47

The intermediate compound 37-2 was obtained by using the intermediate compound 36-2 in place of the intermediate compound 36-1 according to the method described in the Reference Preparation Example 46.

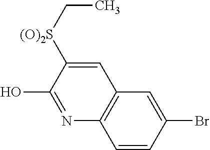

Intermediate compound 37-2: ¹H-NMR (DMSO-D₆) δ: 8.71 (1H, s), 8.28 (1H, d), 7.84 (1H, dd), 7.34 (1H, d), 3.52 (2H, q), 1.15 (3H, t).

### Reference Preparation Example 48

To a mixture of the intermediate compound 37-1 13.1 g and toluene 100 mL was added phosphorus oxychloride 16.9 mL, and the mixture was stirred at 110° C. for 11 hours. The resulting mixture was concentrated, and the obtained solids were washed with water, and then washed with a mixed solvents of MTBE:hexane=1:4, and concentrated under reduced pressure to obtain the intermediate compound 38-1 13.35 g.

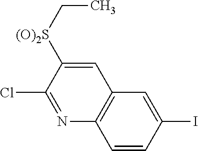

Intermediate compound 38-1: ¹H-NMR (CDCl₃) δ: 8.88 (1H, s), 8.40 (1H, d), 8.17 (1H, dd), 7.83 (1H, d), 3.58 (2H, q), 1.33 (3H, t).

### Reference Preparation Example 49

The intermediate compound 38-2 was obtained by using the intermediate compound 37-2 in place of the intermediate compound 37-1 according to the method described in the Reference Preparation Example 48.

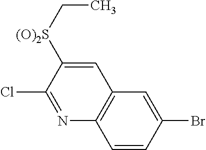

Intermediate compound 38-2: ¹H-NMR (CDCl₃) δ: 8.91 (1H, s), 8.17 (1H, d), 8.01-7.98 (2H, m), 3.59 (2H, q), 1.34 (3H, t).

### Reference Preparation Example 50

A mixture of the intermediate compound 38-1 5.0 g and DMSO 30 mL was added cesium fluoride 3.63 g at room temperature, and the mixture was stirred at 50° C. for 4 hours. Iced water was added to the resulting mixture, and the precipitated out solids were filtered. The obtained solids were washed with water and hexane successively, and dried under reduced pressure to obtain the intermediate compound 39-1 represented by the following formula 4.83 g.

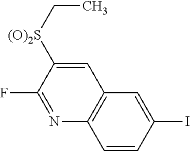

Intermediate compound 39-1: ¹H-NMR (CDCl₃) δ: 8.82 (1H, s), 8.40 (1H, d), 8.15 (1H, dd), 7.76 (1H, d), 3.44 (2H, q), 1.36 (3H, t).

### Reference Preparation Example 50-1

The intermediate compound 39-2 was obtained by using the intermediate compound 38-2 in place of the intermediate compound 38-1 according to the method described in the Reference Preparation Example 50.

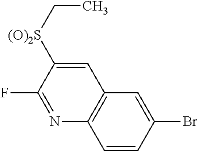

Intermediate compound 39-2: ¹H-NMR (CDCl₃) δ: 8.85 (1H, s), 8.18 (1H, d), 8.00 (1H, dd), 7.91 (1H, d), 3.45 (2H, q), 1.36 (3H, t).

### Reference Preparation Example 51

The intermediate compound 40-1 was obtained by using the intermediate compound 10-1 in place of 4-(trifluoroethyl)antranilic acid according to the method described in the Reference Preparation Example 2.

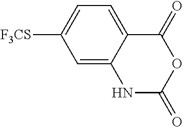

Intermediate compound 40-1: ¹H-NMR (DMSO-D₆) δ: 11.90 (1H, s), 8.03 (1H, d), 7.50 (1H, dd), 7.42 (1H, d).

The compounds which were prepared according to the Reference Preparation Example 51 and their physical property values were shown below.

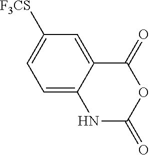

Intermediate compound 40-2: ¹H-NMR (DMSO-D₆) δ: 12.06 (1H, s), 8.15 (1H, d), 8.03 (1H, dd), 7.28 (1H, d).

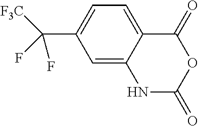 [SMILES: O=c1[nH]c2cc(C(F)(F)C(F)(F)F)ccc2c(=O)o1]

Intermediate compound 40-3: ¹H-NMR (DMSO-D₆) δ: 11.96 (1H, s), 8.15 (1H, d), 7.53 (1H, d), 7.39 (1H, s).

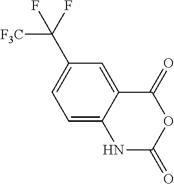 [SMILES: O=c1[nH]c2ccc(C(F)(F)C(F)(F)F)cc2c(=O)o1]

Intermediate compound 40-4: ¹H-NMR (DMSO-D₆) δ: 12.14 (1H, s), 8.08 (1H, d), 8.04 (1H, dd), 7.37 (1H, d).

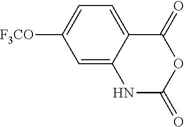 [SMILES: O=c1[nH]c2cc(OC(F)(F)F)ccc2c(=O)o1]

Intermediate compound 40-5: ¹H-NMR (DMSO-D₆) δ: 11.88 (1H, s), 8.05 (1H, d), 7.21 (1H, dd), 7.04 (1H, d).

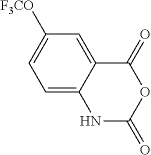 [SMILES: O=c1[nH]c2ccc(OC(F)(F)F)cc2c(=O)o1]

Intermediate compound 40-6: ¹H-NMR (DMSO-D₆) δ: 9.92 (1H, s), 5.83 (1H, d), 5.80-5.77 (1H, m), 5.26 (1H, d).

### Reference Preparation Example 52

The intermediate compound 41-1 was obtained by using the intermediate compound 23-1 in place of 6-bromo-3-(ethylthio)imidazo[1,2-a]pyridine-2-amine according to the method described in the Reference Preparation Example 3.

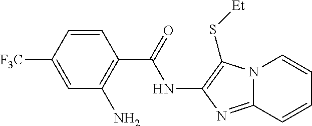 [SMILES: CCSc1c(NC(=O)c2ccc(C(F)(F)F)cc2N)nc2ccccn12]

Intermediate compound 41-1: ¹H-NMR (CDCl₃) δ: 8.40-8.39 (2H, m), 7.67 (1H, s), 7.65 (1H, s), 7.34-7.29 (1H, m), 6.99-6.92 (3H, m), 5.91 (2H, s), 2.70 (2H, q), 1.22 (3H, t).

The compounds which were prepared according to the Reference Preparation Example 52 and their physical property values were shown below.

A compound represented by formula (C-1):

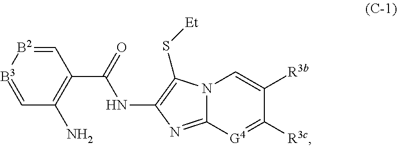

wherein a combination of B², B³, G⁴, R^{3b} and R^{3c} represents any combinations indicated in [Table C-1].

TABLE C-1

| Intermediate | | | | | |
|---|---|---|---|---|---|
| compound | B² | B³ | G⁴ | R^{3b} | R^{3c} |
| 41-2 | CH | CCF₃ | CH | F | H |
| 41-3 | CH | CCF₃ | CH | Cl | H |
| 41-4 | CH | CCF₃ | CH | I | H |
| 41-5 | CH | CCF₃ | CH | CF₃ | H |
| 41-6 | CH | CCF₃ | CH | Me | H |
| 41-7 | CH | CCF₃ | CH | H | I |
| 41-8 | CCF₃ | CH | CH | H | H |
| 41-9 | CCF₃ | CH | CH | Cl | H |
| 41-10 | CCF₃ | CH | CH | I | H |
| 41-11 | CCF₃ | CH | CH | CF₃ | H |
| 41-12 | CCF₃ | CH | CH | H | I |
| 41-13 | CCF₃ | CH | CH | H | CF₃ |
| 41-14 | CH | CSCF₃ | CH | I | H |
| 41-15 | CSCF₃ | CH | CH | I | H |
| 41-16 | COCF₃ | CH | CH | H | H |
| 41-17 | CH | COCF₃ | CH | I | H |
| 41-18 | COCF₃ | CH | CH | I | H |
| 41-19 | CH | CC₂F₅ | CH | I | H |
| 41-20 | CC₂F₅ | CH | CH | I | H |
| 41-21 | CH | CCF₃ | N | Br | H |
| 41-22 | CCF₃ | CH | N | Br | H |
| 41-23 | CH | CCF₃ | N | I | H |
| 41-24 | CCF₃ | CH | N | I | H |
| 41-25 | COCF₃ | CH | N | Br | H |
| 41-26 | COCF₃ | CH | N | I | H |
| 41-27 | COCF₃ | CH | N | H | H |

Intermediate compound 41-2: ¹H-NMR (CDCl₃) δ: 8.34-8.31 (2H, m), 7.66-7.64 (2H, m), 7.25-7.20 (1H, m), 6.98-6.93 (2H, m), 5.92 (2H, s), 2.72 (2H, q), 1 0.23 (3H, t).

Intermediate compound 41-3: ¹H-NMR (CDCl₃) δ: 8.41-8.40 (1H, m), 8.29 (1H, s), 7.64-7.62 (2H, m), 7.30-7.28 (1H, m), 6.96-6.92 (2H, m), 5.92 (2H, s), 2.72 (2H, q), 1 0.23 (3H, t).

Intermediate compound 41-4: ¹H-NMR (CDCl₃) δ: 8.62-8.60 (1H, m), 8.35 (1H, s), 7.64 (1H, d), 7.50 (1H, dd), 7.47-7.44 (1H, m), 6.98-6.95 (1H, m), 6.95-6.93 (1H, m), 5.92 (2H, s), 2.72 (2H, q), 1.24 (3H, t).

Intermediate compound 41-5: ¹H-NMR (CDCl₃) δ: 8.88-8.87 (1H, m), 8.52 (1H, d), 8.22 (1H, s), 8.09 (1H, s), 7.83 (1H, d), 7.79 (1H, dd), 7.59 (1H, dd), 2.80 (2H, q), 1.20 (3H, t).

Intermediate compound 41-6: ¹H-NMR (DMSO-D₆) δ: 10.31 (1H, s), 8.39-8.38 (1H, m), 7.89 (1H, d), 7.52-7.51 (1H, m), 7.27 (1H, dd), 7.11-7.10 (1H, m), 6.85 (1H, dd), 6.75 (2H, s), 2.72 (2H, q), 2.38 (3H, s), 1.07 (3H, t).

Intermediate compound 41-7: ¹H-NMR (CDCl₃) δ: 8.60 (1H, s), 8.18 (1H, dd), 8.07-8.06 (1H, m), 7.86-7.84 (1H, m), 7.50 (1H, dd), 7.25-7.23 (1H, m), 6.78 (1H, d), 6.14 (2H, s), 2.74 (2H, q), 1.25 (3H, t).

Intermediate compound 41-8: ¹H-NMR (CDCl₃) δ: 8.41-8.40 (1H, m), 8.31 (1H, s), 7.82-7.79 (1H, m), 7.67 (1H, d), 7.48 (1H, dd), 7.34-7.30 (1H, m), 6.98-6.96 (1H, m), 6.77 (1H, d), 6.12 (2H, s), 2.72 (2H, q), 1.26 (3H, t).

Intermediate compound 41-9: ¹H-NMR (CDCl₃) δ: 8.43-8.41 (1H, m), 8.28 (1H, s), 7.80-7.77 (1H, m), 7.61 (1H, d), 7.49 (1H, dd), 7.30-7.27 (1H, m), 6.77 (1H, d), 6.12 (2H, s), 2.73 (2H, q), 1.26 (3H, t).

Intermediate compound 41-10: ¹H-NMR (CDCl₃) δ: 8.64-8.61 (1H, m), 8.46-8.43 (1H, m), 7.81 (1H, s), 7.50-7.48 (3H, m), 6.77 (1H, d), 6.13 (2H, s), 2.74 (2H, q), 1.26 (3H, t).

Intermediate compound 41-11: ¹H-NMR (CDCl₃) δ: 8.74 (1H, s), 8.46 (1H, s), 7.80 (1H, s), 7.76 (1H, d), 7.50-7.45 (2H, m), 6.77 (1H, d), 6.14 (2H, s), 2.76 (2H, q), 1.26 (3H, t).

Intermediate compound 41-12: ¹H-NMR (CDCl₃) δ: 8.39 (1H, s), 8.14-8.12 (1H, m), 8.06-8.05 (1H, m), 7.65 (1H, d), 7.21 (1H, dd), 6.95-6.93 (2H, m), 5.91 (2H, s), 2.70 (2H, q), 1.21 (3H, t).

Intermediate compound 41-13: ¹H-NMR (CDCl₃) δ: 8.50 (1H, d), 8.48 (1H, s), 7.95 (1H, s), 7.82-7.79 (1H, m), 7.48 (1H, dd), 7.14 (1H, dd), 6.76 (1H, d), 6.13 (2H, s), 2.75 (2H, q), 1.25 (3H, t).

Intermediate compound 41-14: ¹H-NMR (CDCl₃) δ: 8.61-8.60 (1H, m), 8.42 (1H, s), 7.56 (1H, d), 7.50-7.44 (2H, m), 7.00 (1H, s), 6.95 (1H, d), 5.87 (2H, s), 2.72 (2H, q), 1.23 (3H, t).

Intermediate compound 41-15: ¹H-NMR (CDCl₃) δ: 8.60 (1H, s), 8.37 (1H, s), 7.80 (1H, s), 7.47-7.42 (3H, m), 6.71 (1H, d), 6.09 (2H, s), 2.72 (2H, q), 1.25 (3H, t).

Intermediate compound 41-16: ¹H-NMR (CDCl₃) δ: 8.41-8.39 (1H, m), 8.27 (1H, s), 7.67 (1H, d), 7.42-7.41 (1H, m), 7.34-7.29 (1H, m), 7.18-7.15 (1H, m), 6.98-6.94 (1H, m), 6.72 (1H, d), 5.77 (2H, s), 2.71 (2H, q), 1.23 (3H, t).

Intermediate compound 41-17: ¹H-NMR (CDCl₃) δ: 8.61-8.60 (1H, m), 8.28 (1H, s), 7.56 (1H, d), 7.50-7.44 (2H, m), 6.57-6.54 (2H, m), 5.96 (2H, s), 2.71 (2H, q), 1.23 (3H, t).

Intermediate compound 41-18: ¹H-NMR (CDCl₃) δ: 8.62-8.61 (1H, m), 8.30 (1H, s), 7.49-7.45 (2H, m), 7.40-7.39 (1H, m), 7.19-7.16 (1H, m), 6.72 (1H, d), 5.78 (2H, s), 2.72 (2H, q), 1.24 (3H, t).

Intermediate compound 41-19: ¹H-NMR (CDCl₃) δ: 8.61-8.60 (1H, m), 8.32 (1H, s), 7.64 (1H, d), 7.50-7.45 (2H, m), 6.94-6.91 (2H, m), 5.92 (2H, s), 2.72 (2H, q), 1.24 (3H, t).

Intermediate compound 41-20: ¹H-NMR (CDCl₃) δ: 8.62 (1H, s), 8.27 (1H, s), 7.73 (1H, s), 7.51-7.45 (3H, m), 6.79 (1H, d), 6.14 (2H, s), 2.72 (2H, q), 1.25 (3H, t).

Intermediate compound 41-21: ¹H-NMR (CDCl₃) δ: 8.90 (1H, s), 8.80 (1H, d), 8.59 (1H, d), 7.79 (1H, d), 6.96-6.90 (2H, m), 5.90 (2H, s), 2.80 (2H, q), 1.23 (3H, t).

Intermediate compound 41-22: ¹H-NMR (CDCl₃) δ: 8.80 (1H, d), 8.58 (1H, d), 8.51 (1H, s), 7.84 (1H, s), 7.49 (1H, d), 6.77 (1H, d), 6.10 (2H, s), 2.80 (2H, q), 1.26 (3H, t).

Intermediate compound 41-23: ¹H-NMR (CDCl₃) δ: 8.93 (1H, s), 8.88 (1H, d), 8.66 (1H, d), 7.82 (1H, d), 6.95 (1H, s), 6.91 (1H, d), 5.90 (2H, s), 2.79 (2H, q), 1.27 (3H, t).

Intermediate compound 41-24: ¹H-NMR (CDCl₃) δ: 8.89 (1H, d), 8.66 (1H, d), 8.45 (1H, s), 7.83 (1H, s), 7.49 (1H, d), 6.77 (1H, d), 6.10 (2H, s), 2.79 (2H, q), 1.26 (3H, t).

Intermediate compound 41-25: ¹H-NMR (CDCl₃) δ: 8.79 (1H, d), 8.65 (1H, s), 8.57 (1H, d), 7.51 (1H, s), 7.17 (1H, d), 6.71 (1H, d), 5.77 (2H, s), 2.79 (2H, q), 1.25 (3H, t).

Intermediate compound 41-26: ¹H-NMR (CDCl₃) δ: 8.88 (1H, d), 8.68 (1H, s), 8.65 (1H, d), 7.51 (1H, s), 7.17 (1H, d), 6.71 (1H, d), 5.78 (2H, s), 2.79 (2H, q), 1.26 (3H, t).

Intermediate compound 41-27: ¹H-NMR (CDCl₃) δ: 8.54 (1H, dd), 8.12 (1H, dd), 7.78 (1H, s), 7.49 (1H, s), 7.31-7.21 (1H, m), 6.91 (1H, dd), 6.76 (1H, d), 5.72 (2H, s), 3.25 (2H, q), 1.38 (3H, t).

### Reference Preparation Example 53

The intermediate compound 42 was obtained by using ethyl 6-fluoroimidazo[1,2-a]pyridine-2-carboxylate in place of the intermediate compound 5 according to the method described in the Reference Preparation Example 6.

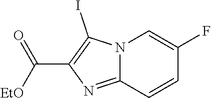 [SMILES: CCOC(=O)c1nc2ccc(F)cn2c1I]

Intermediate compound 42: ¹H-NMR (CDCl₃) δ: 8.24-8.23 (1H, m), 7.71-7.68 (1H, m), 7.28-7.25 (1H, m), 4.50 (2H, q), 1.48 (3H, t).

### Reference Preparation Example 54

A mixture of the intermediate compound 35-2 1.00 g, hydrazine monohydrate 0.48 mL and ethanol 5 mL was stirred at 80° C. for 15 minutes. The resulting mixture was stood to cool to room temperature, and water was added thereto, and the resulting solids were filtered, and washed with water, and dried under reduced pressure to obtain the intermediate compound 43 represented by the following formula 0.73 g.

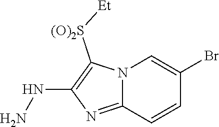

Intermediate compound 43: ¹H-NMR (CDCl₃) δ: 8.69 (1H, dd), 7.46 (1H, dd), 7.36 (1H, dd), 6.74 (1H, s), 3.98 (2H, s), 3.19 (2H, q), 1.32 (3H, t).

### Preparation Example 1

To a mixture of the intermediate 1 0.50 g, 3-(ethanesulfonyl)-2-fluoropyridine 0.49 g and NMP 5 mL was added sodium hydride (60%, dispersion in mineral oil) 103 mg under nitrogen atmosphere, and the mixture was stirred at 110° C. for 1 hour. The resulting mixture was stood to cool to room temperature, and water was added thereto, and the mixture was filtered. The obtained solids were washed with water, and dried under reduced pressure to obtain the present compound 1-1 represented by the following formula 0.54 g.

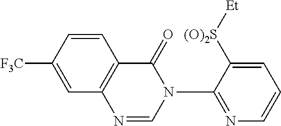

Present compound 1-1: ¹H-NMR (CDCl₃) δ: 8.94-8.93 (1H, m), 8.54-8.52 (1H, m), 8.45 (1H, d), 8.13 (1H, s), 8.11 (1H, s), 7.80-7.77 (2H, m), 3.36-3.34 (2H, m), 1.34 (3H, t).

### Preparation Example 2

The present compound 1-2 was obtained by using 5-cyclopropyl-3-(ethanesulfonyl)-2-fluoropyridine in place of 3-(ethanesulfonyl)-2-fluoropyridine according to the Preparation Example 1.

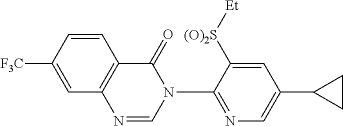

Present compound 1-2: ¹H-NMR (CDCl₃) δ: 8.65 (1H, d), 8.44 (1H, d), 8.09 (1H, d), 8.08 (1H, s), 8.05 (1H, d), 7.76 (1H, dd), 3.30-3.28 (2H, m), 2.14-2.09 (1H, m), 1.31 (3H, t), 1.29-1.24 (2H, m), 0.98-0.92 (2H, m).

### Preparation Example 3

A mixture of the intermediate compound 3-1 491 mg and triethyl orthoformate 11 mL was stirred at 100° C. for 1 hour. The resulting mixture was stood to cool to room temperature and concentrated. The obtained solids were washed with hexane to obtain the present compound 2-1 represented by the following formula 447 mg.

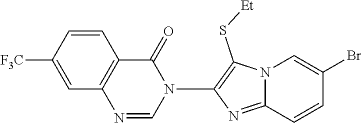 [SMILES: CCSc1c(-n2cnc3cc(C(F)(F)F)ccc3c2=O)nc2ccc(Br)cn12]

Present compound 2-1: ¹H-NMR (CDCl₃) δ: 8.65 (1H, dd), 8.51 (1H, d), 8.20 (1H, s), 8.08 (1H, d), 7.77 (1H, dd), 7.61 (1H, dd), 7.50 (1H, dd), 2.77 (2H, q), 1.18 (3H, t).

### Preparation Example 4

The compounds which were prepared according to the Preparation Example 3 and their physical property values were shown below.

A compound represented by formula (A-1):

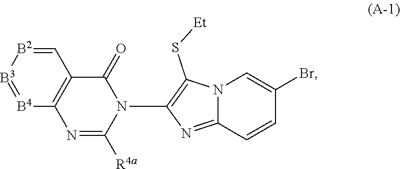

wherein a combination of R^{4a}, B², B³ and B⁴ represents any combinations indicated in [Table A-1].

TABLE A-1

| Present | | | | |
|---|---|---|---|---|
| compound | R^{4a} | B² | B³ | B⁴ |
| 2-2 | H | CCF₃ | CH | CH |
| 2-3 | H | CH | CH | CCF₃ |
| 2-4 | H | CH | CCF₃ | N |
| 2-5 | H | CH | CCl | CH |
| 2-6 | H | CH | CI | CH |
| 2-7 | H | COCF₃ | CH | CH |

Present compound 2-2: ¹H-NMR (CDCl₃) δ: 8.69-8.67 (1H, m), 8.65-8.64 (1H, m), 8.22 (1H, s), 8.03 (1H, dd), 7.92-7.90 (1H, m), 7.61 (1H, dd), 7.52-7.49 (1H, m), 2.78 (2H, q), 1.19 (3H, t).

Present compound 2-3: ¹H-NMR (CDCl₃) δ: 8.64 (1H, d), 8.60 (1H, d), 8.25 (1H, s), 8.15 (1H, d), 7.63 (1H, t), 7.61 (1H, d), 7.50 (1H, dd), 2.77 (2H, q), 1.19 (3H, t).

Present compound 2-4: ¹H-NMR (CDCl₃) δ: 8.93 (1H, d), 8.66 (1H, d), 8.46 (1H, s), 7.89 (1H, d), 7.63 (1H, d), 7.53 (1H, dd), 2.77 (2H, q), 1.19 (3H, t).

Present compound 2-5: ¹H-NMR (CDCl₃) δ: 8.65-8.63 (1H, m), 8.32 (1H, d), 8.14 (1H, s), 7.79 (1H, d), 7.60 (1H, dd), 7.52 (1H, dd), 7.49 (1H, dd), 2.76 (2H, q), 1.18 (3H, t).

Present compound 2-6: ¹H-NMR (CDCl₃) δ: 8.64 (1H, dd), 8.21 (1H, d), 8.11 (1H, s), 8.06 (1H, d), 7.88 (1H, dd), 7.60 (1H, dd), 7.49 (1H, dd), 2.75 (2H, q), 1.17 (3H, t).

Present compound 2-7: ¹H-NMR (CDCl₃) δ: 8.67-8.64 (1H, m), 8.23-8.20 (1H, m), 8.16 (1H, s), 7.87-7.85 (1H, m), 7.67-7.59 (2H, m), 7.51-7.49 (1H, m), 2.77 (2H, q), 1.19 (3H, t).

A compound represented by formula (A-1-2):

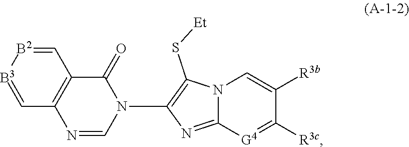

wherein a combination of B², B³, G⁴, R^{3b} and R^{3c} represents any combinations indicated in [Table A-1-2].

TABLE A-1-2

| Present | | | | | |
|---|---|---|---|---|---|
| compound | B² | B³ | G⁴ | R^{3b} | R^{3c} |
| 2-8 | CH | CCF₃ | CH | H | H |
| 2-9 | CH | CCF₃ | CH | F | H |
| 2-10 | CH | CCF₃ | CH | Cl | H |
| 2-11 | CH | CCF₃ | CH | I | H |
| 2-12 | CH | CCF₃ | CH | CF₃ | H |
| 2-13 | CH | CCF₃ | CH | Me | H |
| 2-14 | CH | CCF₃ | CH | H | I |
| 2-16 | CCF₃ | CH | CH | H | H |
| 2-17 | CCF₃ | CH | CH | Cl | H |
| 2-18 | CCF₃ | CH | CH | I | H |
| 2-19 | CCF₃ | CH | CH | CF₃ | H |
| 2-20 | CCF₃ | CH | CH | H | I |
| 2-21 | CCF₃ | CH | CH | H | CF₃ |
| 2-22 | CH | CSCF₃ | CH | I | H |
| 2-23 | CSCF₃ | CH | CH | I | H |
| 2-24 | COCF₃ | CH | CH | H | H |
| 2-25 | CH | COCF₃ | CH | I | H |
| 2-26 | COCF₃ | CH | CH | I | H |
| 2-27 | CH | CC₂F₅ | CH | I | H |
| 2-28 | CC₂F₅ | CH | CH | I | H |
| 2-29 | CH | CCF₃ | N | Br | H |
| 2-30 | CCF₃ | CH | N | Br | H |
| 2-31 | CH | CCF₃ | N | I | H |
| 2-32 | CCF₃ | CH | N | I | H |
| 2-33 | COCF₃ | CH | N | Br | H |
| 2-34 | COCF₃ | CH | N | I | H |
| 2-37 | COCF₃ | CH | N | H | H |

Present compound 2-8: ¹H-NMR (CDCl₃) δ: 8.53-8.52 (2H, m), 8.23 (1H, s), 8.08 (1H, s), 7.77 (1H, dd), 7.73-7.70 (1H, m), 7.45-7.43 (1H, m), 7.10-7.09 (1H, m), 2.75 (2H, q), 1.17 (3H, t).

Present compound 2-9: ¹H NMR (CDCl₃) δ: 8.51 (1H, d), 8.46-8.45 (1H, m), 8.21 (1H, s), 8.08-8.08 (1H, m), 7.77 (1H, dd), 7.72-7.68 (1H, m), 7.39-7.34 (1H, m), 2.76 (2H, q), 1.18 (3H, t).

Present compound 2-10: ¹H NMR (CDCl₃) δ: 8.56-8.53 (1H, m), 8.51 (1H, d), 8.20 (1H, s), 8.08 (1H, s), 7.79-7.77 (1H, m), 7.68-7.65 (1H, m), 7.41 (1H, dd), 2.77 (2H, q), 1.18 (3H, t).

Present compound 2-11: ¹H-NMR (CDCl₃) δ: 8.75-8.74 (1H, m), 8.51 (1H, d), 8.20 (1H, s), 8.08 (1H, s), 7.77 (1H, dd), 7.61 (1H, dd), 7.50 (1H, dd), 2.76 (2H, q), 1.18 (3H, dd).

Present compound 2-12: ¹H-NMR (CDCl₃) δ: 8.89-8.87 (1H, m), 8.51 (1H, d), 8.21 (1H, s), 8.10-8.08 (1H, m), 7.83 (1H, d), 7.79 (1H, dd), 7.59 (1H, dd), 2.80 (2H, q), 1.20 (3H, t).

Present compound 2-13: ¹H-NMR (CDCl₃) δ: 8.51 (1H, d), 8.30-8.28 (1H, m), 8.21 (1H, s), 8.07 (1H, s), 7.76 (1H, dd), 7.61 (1H, d), 7.28 (1H, dd), 2.73 (2H, q), 2.45 (3H, s), 1.17 (3H, t).

Present compound 2-14: ¹H-NMR (CDCl₃) δ: 8.51 (1H, d), 8.26 (1H, dd), 8.20 (1H, s), 8.12-8.11 (1H, m), 8.08 (1H, s), 7.77 (1H, dd), 7.33 (1H, dd), 2.74 (2H, q), 1.17 (3H, t).

Present compound 2-16: ¹H-NMR (CDCl₃) δ: 8.69-8.68 (1H, m), 8.54-8.52 (1H, m), 8.25 (1H, s), 8.03 (1H, dd), 7.91 (1H, d), 7.71 (1H, d), 7.45-7.43 (1H, m), 7.10-7.09 (1H, m), 2.76 (2H, q), 1.18 (3H, t).

Present compound 2-17: ¹H-NMR (CDCl₃) δ: 8.69-8.67 (1H, m), 8.56-8.55 (1H, m), 8.22 (1H, s), 8.03 (1H, dd), 7.92-7.90 (1H, m), 7.66 (1H, dd), 7.41 (1H, dd), 2.78 (2H, q), 1.19 (3H, t).

Present compound 2-18: ¹H-NMR (CDCl₃) δ: 8.76-8.74 (1H, m), 8.69-8.67 (1H, m), 8.22 (1H, s), 8.03 (1H, dd), 7.91 (1H, d), 7.62 (1H, dd), 7.50 (1H, d), 2.77 (2H, q), 1.19 (3H, t).

Present compound 2-19: ¹H-NMR (CDCl₃) δ: 8.89-8.87 (1H, m), 8.69-8.68 (1H, m), 8.24 (1H, s), 8.04 (1H, dd), 7.93 (1H, d), 7.83 (1H, d), 7.59 (1H, dd), 2.81 (2H, q), 1.21 (3H, t).

Present compound 2-20: ¹H-NMR (CDCl₃) δ: 8.69-8.66 (1H, m), 8.27-8.25 (1H, m), 8.22 (1H, s), 8.13-8.12 (1H, m), 8.03 (1H, dd), 7.92-7.90 (1H, m), 7.33 (1H, dd), 2.76 (2H, q), 1.18 (3H, t).

Present compound 2-21: ¹H-NMR (CDCl₃) δ: 8.69-8.68 (1H, m), 8.65 (1H, d), 8.24 (1H, s), 8.05-8.04 (2H, m), 7.93 (1H, d), 7.28-7.26 (1H, m), 2.81 (2H, q), 1.20 (3H, t).

Present compound 2-22: ¹H-NMR (CDCl₃) δ: 8.76-8.74 (1H, m), 8.41 (1H, d), 8.17 (1H, s), 8.09 (1H, d), 7.77 (1H, dd), 7.61 (1H, dd), 7.49 (1H, dd), 2.76 (2H, q), 1.19 (3H, t).

Present compound 2-23: ¹H-NMR (CDCl₃) δ: 8.76-8.74 (1H, m), 8.68 (1H, d), 8.20 (1H, s), 8.05 (1H, dd), 7.84 (1H, d), 7.61 (1H, dd), 7.49 (1H, dd), 2.78 (2H, q), 1.19 (3H, t).

Present compound 2-24: ¹H-NMR (CDCl₃) δ: 8.54-8.52 (1H, m), 8.23-8.21 (1H, m), 8.18 (1H, s), 7.85 (1H, d), 7.72-7.70 (1H, m), 7.66 (1H, dd), 7.46-7.41 (1H, m), 7.10-7.08 (1H, m), 2.76 (2H, q), 1.18 (3H, t).

Present compound 2-25: ¹H-NMR (CDCl₃) δ: 8.76-8.74 (1H, m), 8.43 (1H, d), 8.16 (1H, s), 7.62-7.59 (2H, m), 7.49 (1H, dd), 7.39-7.37 (1H, m), 2.76 (2H, q), 1.18 (3H, t).

Present compound 2-26: ¹H-NMR (CDCl₃) δ: 8.76-8.74 (1H, m), 8.22-8.20 (1H, m), 8.14 (1H, s), 7.85 (1H, d), 7.66 (1H, dd), 7.61 (1H, dd), 7.49 (1H, d), 2.77 (2H, q), 1.19 (3H, t).

Present compound 2-27: ¹H-NMR (CDCl₃) δ: 8.76-8.74 (1H, m), 8.52 (1H, d), 8.20 (1H, s), 8.08-8.07 (1H, m), 7.76-7.74 (1H, m), 7.61 (1H, dd), 7.50 (1H, dd), 2.76 (2H, q), 1.18 (3H, t).

Present compound 2-28: ¹H-NMR (CDCl₃) δ: 8.77-8.75 (1H, m), 8.66-8.65 (1H, m), 8.23 (1H, s), 8.00 (1H, dd), 7.92 (1H, d), 7.61 (1H, dd), 7.50 (1H, d), 2.78 (2H, q), 1.19 (3H, t).

Present compound 2-29: ¹H-NMR (CDCl₃) δ: 8.90 (1H, d), 8.73 (1H, d), 8.51 (1H, d), 8.28 (1H, s), 8.10-8.07 (1H, m), 7.79 (1H, dd), 2.82 (2H, q), 1.21 (3H, t).

Present compound 2-30: ¹H-NMR (CDCl₃) δ: 8.91 (1H, d), 8.73 (1H, d), 8.70-8.69 (1H, m), 8.31 (1H, s), 8.04 (1H, dd), 7.92 (1H, d), 2.84 (2H, q), 1.22 (3H, t).

Present compound 2-31: ¹H-NMR (CDCl₃) δ: 9.00 (1H, d), 8.81 (1H, d), 8.51 (1H, d), 8.28 (1H, s), 8.10-8.07 (1H, m), 7.78 (1H, dd), 2.81 (2H, q), 1.20 (3H, t).

Present compound 2-32: ¹H-NMR (CDCl₃) δ: 9.00 (1H, d), 8.81 (1H, d), 8.69-8.68 (1H, m), 8.31 (1H, s), 8.04 (1H, dd), 7.92 (1H, d), 2.83 (2H, q), 1.21 (3H, t).

Present compound 2-33: ¹H-NMR (CDCl₃) δ: 8.90 (1H, d), 8.73 (1H, d), 8.23 (1H, s), 8.22-8.21 (1H, m), 7.86 (1H, d), 7.68-7.65 (1H, m), 2.83 (2H, q), 1.22 (3H, t).

Present compound 2-34: ¹H-NMR (CDCl₃) δ: 9.00 (1H, d), 8.80 (1H, d), 8.23 (1H, s), 8.22-8.21 (1H, m), 7.86 (1H, d), 7.67-7.66 (1H, m), 2.83 (2H, q), 1.21 (3H, t).

Present compound 2-37: ¹H-NMR (CDCl₃) δ: 8.65 (1H, dd), 8.19 (1H, d), 8.08 (1H, s), 7.97 (1H, dd), 7.91 (1H, d), 7.72 (1H, dd), 6.98 (1H, dd), 3.30 (2H, q), 1.38 (3H, t).

### Preparation Example 4-1

To a mixture of 7-(trifluoromethyl)-3-(ethylthio)-imidazo[1,2-a]pyridine-2-amine 310 mg which was prepared according to the method described in WO 2016/129684, and THF 2 mL was added dropwise a solution of potassium bis(trimethylsilyl)amide (1 mol/L THF solution) 2.2 mL at −78° C. under nitrogen atmosphere, and the mixture was stirred for 30 minutes. The intermediate compound 2 350 mg was added to the resulting mixture, and the mixture was stirred at room temperature for 30 minutes. Saturated aqueous ammonium chloride solution was added to the resulting mixture, and the mixture was extracted with ethyl acetate. The resulting organic layer was dried over anhydrous sodium sulfate, and dried reduced pressure. Triethyl orthoformate 13 mL was added to the resulting residue, and the mixture was stirred at 100° C. for 1 hour. The resulting mixture was stood to cool to room temperature, and concentrated. The obtained solids were washed with hexane to obtain the present compound 2-15 represented by the following formula 525 mg.

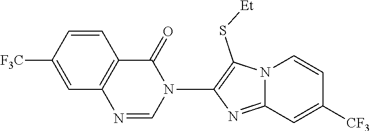 [SMILES: CCSc1c(-n2cnc3cc(C(F)(F)F)ccc3c2=O)nc2cc(C(F)(F)F)ccn12]

Present compound 2-15: ¹H-NMR (CDCl₃) δ: 8.65 (1H, d), 8.51 (1H, d), 8.22 (1H, s), 8.09 (1H, s), 8.03-8.02 (1H, m), 7.79 (1H, dd), 7.27 (1H, dd), 2.79 (2H, q), 1.19 (3H, t).

The compounds which were prepared according to the Preparation Example 4-1 and their physical property values were shown below.

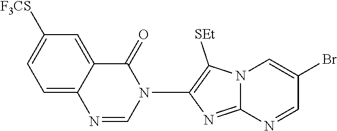

Present compound 2-35: ¹H-NMR (CDCl₃) δ: 8.90 (1H, d), 8.73 (1H, d), 8.69 (1H, d), 8.29 (1H, s), 8.06 (1H, dd), 7.85 (1H, d), 2.84 (2H, q), 1.22 (3H, t).

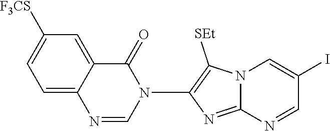

Present compound 2-36: ¹H-NMR (CDCl₃) δ: 9.00 (1H, d), 8.80 (1H, d), 8.69 (1H, d), 8.28 (1H, s), 8.06 (1H, dd), 7.85 (1H, d), 2.83 (2H, q), 1.23 (3H, d).

### Preparation Example 5

To a mixture of the present compound 2-1 252 mg and chloroform 11 mL was added mCPBA (purity 70%, 30% water content) 296 mg under ice-cooling, and the mixture was stirred under ice-cooling for 8 hours. Saturated aqueous solution of sodium hydrogen carbonate and an aqueous solution of sodium thiosulfate were added to the resulting mixture successively, and the mixture was extracted with chloroform. The resulting organic layer was dried over anhydrous sodium sulfate, and concentrated under reduced pressure. The resulting residue was subjected to a silica gel column chromatography (hexane:ethylacetate=1 2) to obtain the present compound 3-1 178 mg.

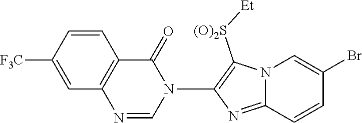

Present compound 3-1: ¹H-NMR (CDCl₃) δ: 9.06 (1H, dd), 8.47 (1H, d), 8.26 (1H, s), 8.09 (1H, d), 7.78 (1H, dd), 7.73 (1H, dd), 7.68 (1H, dd), 3.56 (2H, q), 1.46 (3H, t).

### Preparation Example 6

The compounds which were prepared according to the Preparation Example 5 and their physical property values were shown below.

A compound represented by formula (A-2):

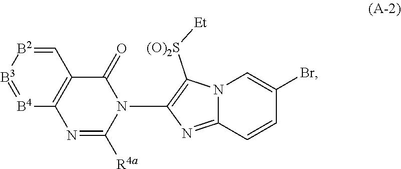

wherein the combination of R^{4a}, B², B³ and B⁴ represents any combinations indicated in [Table A-2].

TABLE A-2

| Present | | | | |
|---|---|---|---|---|
| compound | R^{4a} | B² | B³ | B⁴ |
| 3-2 | H | CCF₃ | CH | CH |
| 3-3 | H | CH | CH | CCF₃ |
| 3-4 | H | CH | CCF₃ | N |
| 3-5 | H | CH | CCl | CH |
| 3-6 | H | CH | CI | CH |
| 3-7 | H | COCF₃ | CH | CH |

Present compound 3-2: ¹H-NMR (CDCl₃) δ: 9.06-9.05 (1H, m), 8.64-8.63 (1H, m), 8.28 (1H, s), 8.04 (1H, dd), 7.93-7.91 (1H, m), 7.73 (1H, dd), 7.69 (1H, dd), 3.59 (2H, q), 1.47 (3H, t).

Present compound 3-3: ¹H-NMR (CDCl₃) δ: 9.06 (1H, dd), 8.55 (1H, dd), 8.31 (1H, s), 8.16 (1H, dd), 7.73 (1H, dd), 7.68 (1H, dd), 7.64 (1H, t), 3.58 (2H, q), 1.46 (3H, t).

Present compound 3-4: ¹H-NMR (CDCl₃) δ: 9.04 (1H, dd), 8.88 (1H, d), 8.52 (1H, s), 7.88 (1H, d), 7.74 (1H, dd), 7.70 (1H, dd), 3.56 (2H, q), 1.47 (3H, t).

Present compound 3-5: ¹H-NMR (CDCl₃) δ: 9.05 (1H, dd), 8.27 (1H, d), 8.19 (1H, s), 7.80 (1H, d), 7.72 (1H, dd), 7.67 (1H, dd), 7.52 (1H, dd), 3.56 (2H, q), 1.45 (3H, t).

Present compound 3-6: ¹H-NMR (CDCl₃) δ: 9.05 (1H, dd), 8.22 (1H, d), 8.17 (1H, s), 8.01 (1H, d), 7.88 (1H, dd), 7.72 (1H, dd), 7.67 (1H, dd), 3.56 (2H, q), 1.44 (3H, t).

Present compound 3-7: ¹H-NMR (CDCl₃) δ: 9.07-9.05 (1H, m), 8.20 (1H, s), 8.17-8.15 (1H, m), 7.86 (1H, d), 7.72 (1H, dd), 7.68-7.66 (2H, m), 3.58 (2H, q), 1.46 (3H, t).

A compound represented by formula (A-2-2):

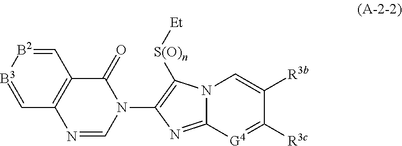

wherein the combination of n, B², B³, G⁴, R^{3b} and R^{3c} represents any combinations indicated in [Table A-2-2].

TABLE A-2-2

| Present | | | | | | |
|---|---|---|---|---|---|---|
| compound | n | B² | B³ | G⁴ | R^{3b} | R^{3c} |
| 3-8 | 2 | CH | CCF₃ | CH | H | H |
| 3-9 | 2 | CH | CCF₃ | CH | F | H |
| 3-10 | 2 | CH | CCF₃ | CH | Cl | H |
| 3-11 | 1 | CH | CCF₃ | CH | I | H |
| 3-12 | 2 | CH | CCF₃ | CH | I | H |
| 3-13 | 2 | CH | CCF₃ | CH | CF₃ | H |
| 3-14 | 2 | CH | CCF₃ | CH | Me | H |
| 3-15 | 2 | CH | CCF₃ | CH | H | I |
| 3-16 | 2 | CH | CCF₃ | CH | H | CF₃ |
| 3-17 | 2 | CCF₃ | CH | CH | H | H |
| 3-18 | 2 | CCF₃ | CH | CH | Cl | H |
| 3-19 | 1 | CCF₃ | CH | CH | I | H |
| 3-20 | 2 | CCF₃ | CH | CH | I | H |
| 3-21 | 2 | CCF₃ | CH | CH | CF₃ | H |
| 3-22 | 2 | CCF₃ | CH | CH | H | I |
| 3-23 | 2 | CCF₃ | CH | CH | H | CF₃ |
| 3-24 | 2 | CH | CSCF₃ | CH | I | H |
| 3-25 | 2 | CSCF₃ | CH | CH | I | H |
| 3-26 | 2 | COCF₃ | CH | CH | H | H |
| 3-27 | 2 | CH | COCF₃ | CH | I | H |
| 3-28 | 2 | COCF₃ | CH | CH | I | H |
| 3-29 | 2 | CH | CC₂F₅ | CH | I | H |
| 3-30 | 2 | CC₂F₅ | CH | CH | I | H |
| 3-31 | 2 | CH | CCF₃ | N | Br | H |
| 3-32 | 2 | CCF₃ | CH | N | Br | H |
| 3-33 | 2 | CH | CCF₃ | N | I | H |
| 3-34 | 2 | CCF₃ | CH | N | I | H |
| 3-35 | 2 | COCF₃ | CH | N | Br | H |
| 3-36 | 2 | COCF₃ | CH | N | I | H |
| 3-37 | 2 | CSCF₃ | CH | N | Br | H |
| 3-38 | 2 | CSCF₃ | CH | N | I | H |
| 3-39 | 2 | COCF₃ | CH | N | H | H |

Present compound 3-8: ¹H-NMR (CDCl₃) δ: 8.93 (1H, d), 8.47 (1H, d), 8.28 (1H, s), 8.08 (1H, s), 7.85-7.83 (1H, m), 7.77 (1H, ad), 7.64-7.61 (1H, m), 7.24-7.22 (1H, m), 3.57-3.52 (2H, m), 1.44 (3H, t).

Present compound 3-9: ¹H-NMR (CDCl₃) δ: 8.91-8.90 (1H, m), 8.47 (1H, di), 8.26 (1H, s), 8.09 (1H, s), 7.83 (1H, dd), 7.79-7.77 (1H, m), 7.56-7.51 (1H, m), 3.56 (2H, q), 1.45 (3H, t).

Present compound 3-10: ¹H-NMR (CDCl₃) δ: 8.97 (1H, dd), 8.47 (1H, d), 8.26 (1H, s), 8.09-8.08 (1H, m), 7.80-7.77 (2H, m), 7.59 (1H, dd), 3.56 (2H, q), 1.46 (3H, t).

Present compound 3-11: ¹H-NMR (CDCl₃) δ: 9.28 (1H, dd), 8.58 (1H, s), 8.45 (1H, d), 8.09 (1H, s), 7.78 (1H, dd), 7.69 (1H, dd), 7.55 (1H, dd), 3.71-3.61 (2H, m), 1.60 (3H, t).

Present compound 3-12: ¹H-NMR (CDCl₃) δ: 9.14-9.13 (1H, m), 8.46 (1H, d), 8.25 (1H, s), 8.10-8.07 (1H, m), 7.80-7.77 (2H, m), 7.61 (1H, dd), 3.56 (2H, q), 1.45 (3H, t).

Present compound 3-13: ¹H-NMR (CDCl₃) δ: 9.30-9.27 (1H, m), 8.47 (1H, d), 8.28 (1H, s), 8.10 (1H, s), 7.96 (1H, d), 7.80-7.75 (2H, m), 3.61 (2H, q), 1.47 (3H, t).

Present compound 3-14: ¹H-NMR (CDCl₃) δ: 8.69-8.67 (1H, m), 8.47 (1H, d), 8.26 (1H, s), 8.08 (1H, s), 7.77 (1H, dd), 7.72 (1H, d), 7.46 (1H, dd), 3.52 (2H, q), 2.48 (3H, s), 1.44 (3H, t).

Present compound 3-15: ¹H-NMR (CDCl₃) δ: 8.65 (1H, dd), 8.47-8.45 (1H, m), 8.26-8.24 (2H, m), 8.09-8.07 (1H, m), 7.78 (1H, dd), 7.46 (1H, dd), 3.58-3.53 (2H, m), 1.43 (3H, t).

Present compound 3-16: ¹H-NMR (CDCl₃) δ: 9.07 (1H, d), 8.47 (1H, d), 8.28 (1H, s), 8.16-8.13 (1H, m), 8.10 (1H, s), 7.79 (1H, dd), 7.39 (1H, dd), 3.63-3.59 (2H, m), 1.47 (3H, t).

Present compound 3-17: ¹H-NMR (CDCl₃) δ: 8.95-8.93 (1H, m), 8.65-8.64 (1H, m), 8.31 (1H, s), 8.04 (1H, dd), 7.93 (1H, d), 7.86-7.83 (1H, m), 7.64-7.62 (1H, m), 7.24-7.23 (1H, m), 3.60-3.56 (2H, m), 1.46 (3H, t).

Present compound 3-18: ¹H-NMR (CDCl₃) δ: 8.97 (1H, dd), 8.64 (1H, d), 8.28 (1H, s), 8.04 (1H, dd), 7.92 (1H, d), 7.78 (1H, dd), 7.59 (1H, dd), 3.59 (2H, q), 1.47 (3H, t).

Present compound 3-19: ¹H-NMR (CDCl₃) δ: 9.28 (1H, dd), 8.62 (1H, d), 8.60 (1H, s), 8.05 (1H, dd), 7.93 (1H, d), 7.69 (1H, dd), 7.55 (1H, dd), 3.72-3.63 (2H, m), 1.62 (3H, t).

Present compound 3-20: ¹H-NMR (CDCl₃) δ: 9.14 (1H, dd), 8.63 (1H, d), 8.28 (1H, s), 8.04 (1H, dd), 7.92 (1H, d), 7.80 (1H, dd), 7.61 (1H, dd), 3.58 (2H, q), 1.47 (3H, t).

Present compound 3-21: ¹H-NMR (CDCl₃) δ: 9.29-9.27 (1H, m), 8.66-8.63 (1H, m), 8.30 (1H, s), 8.05 (1H, dd), 7.98-7.92 (2H, m), 7.77 (1H, dd), 3.64 (2H, q), 1.49 (3H, t).

Present compound 3-22: ¹H-NMR (CDCl₃) δ: 8.65 (1H, dd), 8.64-8.62 (1H, m), 8.27 (1H, s), 8.26-8.25 (1H, m), 8.04 (1H, dd), 7.92 (1H, d), 7.46 (1H, dd), 3.58-3.56 (2H, m), 1.45 (3H, t).

Present compound 3-23: ¹H-NMR (CDCl₃) δ: 9.08-9.06 (1H, m), 8.65-8.63 (1H, m), 8.30 (1H, s), 8.15-8.13 (1H, m), 8.05 (1H, dd), 7.93 (1H, d), 7.39 (1H, dd), 3.64-3.60 (2H, m), 1.48 (3H, t).

Present compound 3-24: ¹H-NMR (CDCl₃) δ: 9.14 (1H, dd), 8.37 (1H, d), 8.23 (1H, s), 8.10 (1H, d), 7.80-7.76 (2H, m), 7.60 (1H, dd), 3.56 (2H, q), 1.45 (3H, t).

Present compound 3-25: ¹H-NMR (CDCl₃) δ: 9.15-9.14 (1H, m), 8.63 (1H, d), 8.25 (1H, s), 8.05 (1H, dd), 7.84 (1H, d), 7.79 (1H, dd), 7.60 (1H, dd), 3.57 (2H, q), 1.46 (3H, t).

Present compound 3-26: ¹H-NMR (CDCl₃) δ: 8.94-8.93 (1H, m), 8.23 (1H, s), 8.18-8.17 (1H, m), 7.86 (1H, d), 7.84-7.82 (1H, m), 7.66 (1H, dd), 7.64-7.59 (1H, m), 7.23-7.21 (1H, m), 3.59-3.55 (2H, m), 1.45 (3H, t).

Present compound 3-27: ¹H-NMR (CDCl₃) δ: 9.15-9.13 (1H, m), 8.38 (1H, d), 8.21 (1H, s), 7.79 (1H, dd), 7.64-7.62 (1H, m), 7.60 (1H, dd), 7.38 (1H, dd), 3.56 (2H, q), 1.45 (3H, t).

Present compound 3-28: ¹H-NMR (CDCl₃) δ: 9.14 (1H, dd), 8.20 (1H, s), 8.17-8.15 (1H, m), 7.85 (1H, d), 7.79 (1H, dd), 7.67-7.65 (1H, m), 7.60 (1H, dd), 3.57 (2H, q), 1.46 (3H, t).

Present compound 3-29: ¹H-NMR (CDCl₃) δ: 9.15-9.13 (1H, m), 8.47 (1H, d), 8.26 (1H, d), 8.07 (1H, s), 7.81-7.78 (1H, m), 7.76-7.74 (1H, m), 7.61 (1H, d), 3.56 (2H, q), 1.47-1.44 (3H, m).

Present compound 3-30: ¹H-NMR (CDCl₃) δ: 9.14 (1H, dd), 8.61 (1H, d), 8.28 (1H, s), 8.01 (1H, dd), 7.93 (1H, d), 7.80 (1H, dd), 7.61 (1H, dd), 3.58 (2H, q), 1.47 (3H, t).

Present compound 3-31: ¹H-NMR (CDCl₃) δ: 9.33 (1H, d), 8.88 (1H, d), 8.47 (1H, d), 8.35 (1H, s), 8.10-8.09 (1H, m), 7.79 (1H, dd), 3.67 (2H, q), 1.49 (3H, t).

Present compound 3-32: ¹H-NMR (CDCl₃) δ: 9.33 (1H, d), 8.88 (1H, d), 8.65-8.63 (1H, m), 8.37 (1H, s), 8.05 (1H, dd), 7.93 (1H, d), 3.69 (2H, q), 1.51 (3H, t).

Present compound 3-33: ¹H-NMR (CDCl₃) δ: 9.41 (1H, d), 8.95 (1H, d), 8.47 (1H, d), 8.35 (1H, s), 8.10-8.08 (1H, m), 7.79 (1H, dd), 3.67 (2H, q), 1.49 (3H, t).

Present compound 3-34: ¹H-NMR (CDCl₃) δ: 9.41 (1H, d), 8.95 (1H, d), 8.64-8.63 (1H, m), 8.37 (1H, s), 8.05 (1H, dd), 7.93 (1H, d), 3.69 (2H, q), 1.50 (3H, t).

Present compound 3-35: ¹H-NMR (CDCl₃) δ: 9.33 (1H, d), 8.87 (1H, d), 8.30 (1H, s), 8.17-8.16 (1H, m), 7.87 (1H, d), 7.69-7.67 (1H, m), 3.69 (2H, q), 1.50 (3H, t).

Present compound 3-36: ¹H-NMR (CDCl₃) δ: 9.41 (1H, d), 8.95 (1H, d), 8.29 (1H, s), 8.17-8.16 (1H, m), 7.86 (1H, d), 7.68-7.67 (1H, m), 3.68 (2H, q), 1.49 (3H, t).

Present compound 3-37: ¹H-NMR (CDCl₃) δ: 9.33 (1H, d), 8.88 (1H, d), 8.64 (1H, d), 8.35 (1H, s), 8.07 (1H, dd), 7.86 (1H, d), 3.69 (2H, q), 1.50 (3H, t).

Present compound 3-38: ¹H-NMR (CDCl₃) δ: 9.41 (1H, d), 8.95 (1H, d), 8.64 (1H, d), 8.35 (1H, s), 8.06 (1H, dd), 7.85 (1H, d), 3.68 (2H, q), 1.50 (3H, t).

Present compound 3-39: ¹H-NMR (CDCl₃) δ: 8.91 (1H, dd), 8.18 (1H, s), 8.16-8.12 (2H, m), 7.92 (1H, d), 7.73 (1H, d), 7.18 (1H, dd), 3.60-3.39 (2H, m), 1.33 (3H, t).

### Preparation Example 7

A mixture of the intermediate compound 6 318 mg, 1,4-dioxane 12 mL, tris(dibenzylideneacetone)dipalladium (0) 55 mg, and Xantphos 69 mg, diisopropylethylamine 0.31 mL, and ethanethiol 0.043 ml was stirred at 50° C. for 2 hours. The resulting mixture was stood to cool to room temperature, and ethanethiol 0.021 mL was added thereto, and the mixture was stirred at 50° C. for 2 hours. The resulting mixture was stood to cool to room temperature, and ethanethiol 0.021 mL was added thereto, and the mixture was stirred at 50° C. for 1.5 hours, and the resulting mixture was then stood to cool to room temperature, and water was added thereto, and the mixture was extracted with ethyl acetate. The resulting organic layer was dried over anhydrous sodium sulfate, and concentrated under reduced pressure. The resulting residue was subjected to a silica gel column chromatography (hexane:ethyl acetate: 1:1) to obtain the present compound 2-1 191 mg.

### Preparation Example 8

To a mixture of the intermediate compound 3-1 300 mg and acetonitrile 4.4 mL were added sodium sulfite 135 mg and iodine 166 mg, and the mixture was stirred at room temperature for 4 hours. Water was added to the resulting mixture, and the mixture was extracted with ethyl acetate. The resulting organic layer was dried over anhydrous sodium sulfate, and concentrated under reduced pressure. The resulting residue was subjected to a silica gel column chromatography (hexane ethyl acetate=1:1) to obtain the present compound 4-1 represented by the following formula 174 mq.

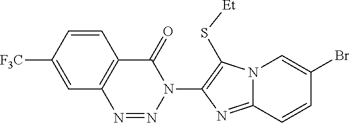 [SMILES: CCSc1c(-n2nnc3cc(C(F)(F)F)ccc3c2=O)nc2ccc(Br)cn12]

Present compound 4-1: ¹H-NMR (CDCl₃) δ: 8.67 (1H, dd), 8.60 (1H, d), 8.56-8.53 (1H, m), 8.09 (1H, dd), 7.64 (1H, dd), 7.50 (1H, dd), 2.78 (2H, q), 1.20 (3H, t).

### Preparation Example 9

The present compound 4-2 represented by the following formula was obtained by using the intermediate compound 3-2 in place of the intermediate compound 3-1 according to the method described in the Preparation Example 8.

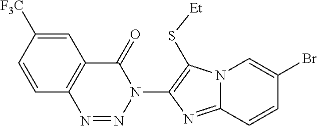 [SMILES: CCSc1c(-n2nnc3ccc(C(F)(F)F)cc3c2=O)nc2ccc(Br)cn12]

Present compound 4-2: ¹H-NMR (CDCl₃) δ: 8.75-8.74 (1H, m), 8.67 (1H, d), 8.41 (1H, d), 8.24 (1H, dd), 7.65 (1H, d), 7.50 (1H, dd), 2.78 (2H, q), 1.20 (3H, t).

### Preparation Example 10

The present compound represented by the following formula 5-1 was obtained by using the present compound 4-1 in place of the present compound 2-1 according to the method described in the Preparation Example 5.

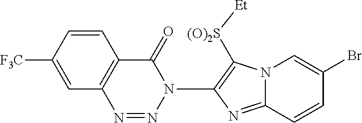

Present compound 5-1: ¹H-NMR (CDCl₃) δ: 9.07 (1H, dd), 8.58 (1H, d), 8.56-8.53 (1H, m), 8.09 (1H, dd), 7.78 (1H, dd), 7.69 (1H, dd), 3.48 (2H, q), 1.44 (3H, t).

### Preparation Example 11

The present compound 5-2 represented by the following formula was obtained by using the present compound 4-2 in place of the present compound 2-1 according to the method described in the Preparation Example 5.

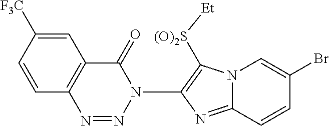

Present compound 5-2: ¹H-NMR (CDCl₃) δ: 9.07-9.06 (1H, m), 8.72-8.71 (1H, m), 8.41 (1H, d), 8.25 (1H, dd), 7.78 (1H, dd), 7.69 (1H, dd), 3.49 (2H, q), 1.45 (3H, t).

### Preparation Example 12

The present compound 6-1 represented by the following formula was obtained by using 6-(trifluoromethyl)isoquinoline-1(2H)-one in place of the intermediate compound 1 and using the intermediate compound 35-2 in place of 3-(ethanesulfonyl)-2-fluoropyridine.

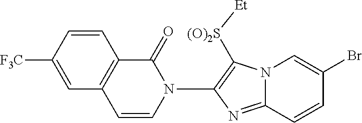

Present compound 6-1: ¹H-NMR (CDCl₃) δ: 9.07 (1H, dd), 8.55 (1H, d), 7.87-7.84 (1H, m), 7.73 (1H, dd), 7.69 (1H, dd), 7.64 (1H, dd), 7.37 (1H, d), 6.69 (1H, d), 3.63 (2H, q), 1.46 (3H, t).

### Preparation Example 13

The compounds which were prepared according to the Preparation Example 12 and their physical property values were shown below.

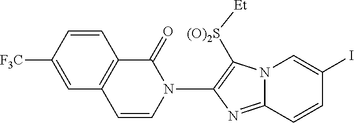

Present compound 6-2: ¹H-NMR (CDCl₃) δ: 9.15 (1H, dd), 8.55 (1H, d), 7.87-7.84 (1H, m), 7.76-7.73 (2H, m), 7.57 (1H, dd), 7.36 (1H, d), 6.69 (1H, d), 3.62 (2H, q), 1.46 (3H, t).

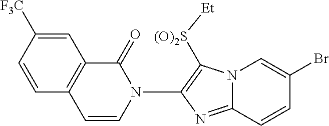

Present compound 6-3: ¹H-NMR (CDCl₃) δ: 9.06 (1H, dd), 8.73-8.70 (1H, m), 7.90 (1H, dd), 7.71-7.67 (2H, m), 7.64 (1H, dd), 7.39 (1H, d), 6.68 (1H, d), 3.68-3.62 (2H, m), 1.48 (3H, t).

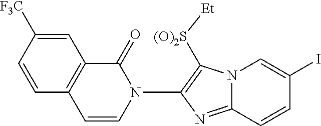

Present compound 6-4: ¹H-NMR (CDCl₃) δ: 9.16-9.15 (1H, m), 8.72-8.70 (1H, m), 7.91-7.89 (1H, m), 7.75 (1H, dd), 7.69 (1H, d), 7.58-7.55 (1H, m), 7.39 (1H, d), 6.68 (1H, d), 3.68-3.61 (2H, q), 1.47 (3H, t).

### Preparation Example 14

The present compound 6-5 represented by the following formula was obtained by using the intermediate compound 38-2 in place of 3-(ethanesulfonyl)-2-fluoropyridine.

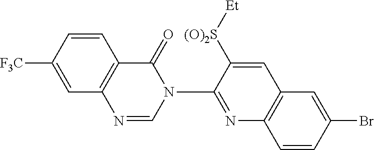

Present compound 6-5: ¹H-NMR (CDCl₃) δ: 8.97 (1H, s), 8.46 (1H, d), 8.31-8.30 (1H, m), 8.20 (1H, s), 8.14-8.13 (1H, m), 8.10-8.09 (2H, m), 7.79 (1H, dd), 3.48-3.34 (2H, m), 1.37 (3H, t).

### Preparation Example 15

The present compound 6-6 represented by the following formula was obtained by using the intermediate compound 38-1 in place of 3-(ethanesulfonyl)-2-fluoropyridine.

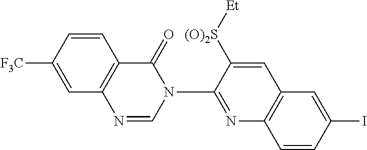

Present compound 6-6: ¹H-NMR (CDCl₃) δ: 8.94 (1H, s), 8.54 (1H, d), 8.46 (1H, d), 8.25 (1H, dd), 8.19 (1H, s), 8.14-8.12 (1H, m), 7.94 (1H, d), 7.79 (1H, dd), 3.47-3.33 (2H, m), 1.36 (3H, t).

### Preparation Example 16

A mixture of the present compound 3-1 0.18 g, cyclopropyl boronic acid 92 mg, [1,1′-bix(diphenylphosphino)ferrocene]dichloropalladium (II) dichloromethane adduct 52 mg, tripotassium phosphate 377 mg, toluene 2.5 mL, and water 0.7 mL was stirred at 100° C. for 6 hours. The resulting mixture was stood to cool to room temperature, and water was added thereto, and the mixture was extracted with ethyl acetate. The resulting organic layer was dried over anhydrous sodium sulfate, and concentrated under reduced pressure. The resulting residue was subjected to a silica gel column chromatography (ethyl acetate:hexane: 2:1) to obtain the present compound 7-1 represented by the following formula 90 mg.

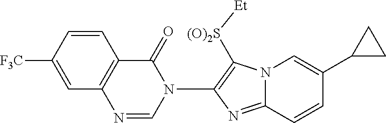

Present compound 7-1: ¹H-NMR (CDCl₃) δ: 8.69 (1H, dd), 8.46 (1H, d), 8.25 (1H, s), 8.09-8.06 (1H, m), 7.76 (1H, dd), 7.71 (1H, dd), 7.29 (1H, dd), 3.52 (2H, q), 2.07-1.99 (1H, m), 1.44 (3H, t), 1.13-1.10 (2H, m), 0.82-0.77 (2H, m).

### Preparation Example 17

The compounds which were prepared according to the Preparation Example 16 and their physical property values were shown below.

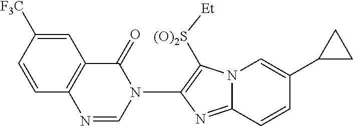

Present compound 7-2: ¹H-NMR (CDCl₃) δ: 8.70-8.69 (1H, m), 8.64-8.62 (1H, m), 8.27 (1H, s), 8.02 (1H, dd), 7.91 (1H, d), 7.71 (1H, dd), 7.29 (1H, dd), 3.57-3.52 (2H, m), 2.05-2.03 (1H, m), 1.45 (3H, t), 1.14-1.09 (2H, m), 0.81-0.78 (2H, m).

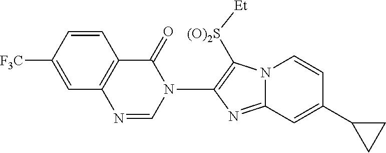

Present compound 7-3: ¹H-NMR (CDCl₃) δ: 8.75 (1H, dd), 8.46 (1H, d), 8.26 (1H, s), 8.09-8.06 (1H, m), 7.76 (1H, dd), 7.46-7.43 (1H, m), 6.88 (1H, dd), 3.54-3.47 (2H, m), 2.07-2.03 (1H, m), 1.41 (3H, t), 1.24-1.19 (2H, m), 0.91-0.87 (2H, m).

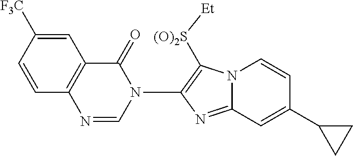

Present compound 7-4: ¹H-NMR (CDCl₃) δ: 8.75 (1H, d), 8.65-8.62 (1H, m), 8.28 (1H, s), 8.02 (1H, dd), 7.91 (1H, d), 7.46-7.44 (1H, m), 6.89 (1H, dd), 3.58-3.48 (2H, m), 2.07-2.02 (1H, m), 1.43 (3H, t), 1.22-1.19 (2H, m), 0.90-0.88 (2H, m)).

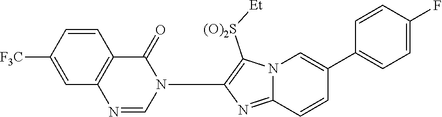

Present compound 7-5: ¹H-NMR (CDCl₃) δ: 9.02 (1H, s), 8.48 (1H, d), 8.29 (1H, s), 8.09 (1H, s), 7.89 (1H, d), 7.81-7.77 (2H, m), 7.60-7.56 (2H, m), 7.25-7.21 (2H, m), 3.57 (2H, q), 1.46 (3H, t).

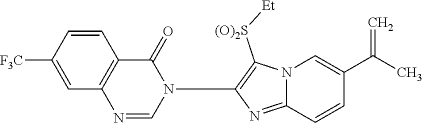

Present compound 7-6: ¹H-NMR (CDCl₃) δ: 8.90 (1H, s), 8.47 (1H, d), 8.27 (1H, s), 8.08 (1H, s), 7.81-7.74 (3H, m), 5.55 (1H, s), 5.34 (1H, s), 3.58-3.52 (2H, m), 2.23 (3H, s), 1.45 (3H, t).

### Reference Preparation Example 55

A mixture of the present compound 3-1 2.0 g, bis(pinacolato)diboron 1.1 g, [1,1′-bis(diphenylphosphino)ferrocene]dichloropalladium(II) dichloromethane adduct 0.086 g, potassium acetate 1.17 g, and toluene 20 mL was stirred at 120° C. for 8 hours. The resulting mixture was stood to cool to room temperature, and water was added thereto, and the mixture was extracted with chloroform. The resulting organic layer was dried over anhydrous magnesium sulfate, and dried under reduced pressure to obtain the intermediate compound 47 represented by the following formula 1.9 g.

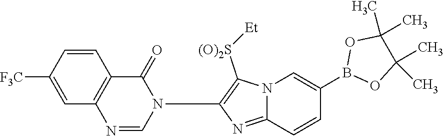

Intermediate compound 47: ¹H-NMR (CDCl₃) δ: 9.17 (1H, s), 8.47 (1H, d), 8.28 (1H, s), 8.08 (1H, s), 7.89 (1H, dd), 7.80-7.75 (2H, m), 3.55 (2H, q), 1.45 (3H, t), 1.38 (12H, s).

### Preparation Example 19

A mixture of the intermediate compound 47 300 mg, 2-bromopyridine 130 mg, [1,1′-bis(diphenylphosphino)ferrocene]dichloropalladium(II) dichloromethane adduct 8.0 mg, tripotassium phosphate 350 mg, dimethoxyethane 3.0 mL, and water 0.3 mL was stirred at 80° C. for 4 hours. The resulting mixture was stood to cool to room temperature, and water was then added thereto, and the mixture was extracted with ethyl acetate. The resulting organic layer was dried over anhydrous sodium sulfate, and concentrated under reduced pressure. The resulting residue was subjected to a silica gel column chromatography (ethyl acetate:hexane: 4:1) to obtain the present compound 8-1 represented by the following formula 190 mg.

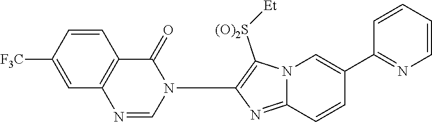

Present compound 8-1: ¹H-NMR (CDCl₃) δ: 9.55 (1H, s), 8.77 (1H, d), 8.48 (1H, d), 8.31-8.27 (2H, m), 8.09 (1H, s), 7.93-7.84 (2H, m), 7.82-7.76 (2H, m), 7.40-7.35 (1H, m), 3.60 (2H, q), 1.48 (3H, t).

### Preparation Example 20

The present compound 8-2 represented by the following formula was obtained by using 2-bromopyrimidine in place of 2-bromopyridine.

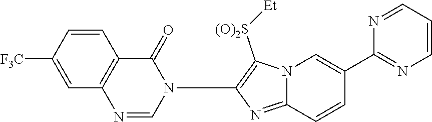

Present compound 8-2: ¹H-NMR (CDCl₃) δ: 10.02 (1H, s), 8.87 (2H, d), 8.69 (1H, dd), 8.48 (1H, d), 8.32 (1H, s), 8.10 (1H, s), 7.90 (1H, dd), 7.78 (1H, dd), 7.33 (1H, t), 3.62 (2H, q), 1.50 (3H, t).

### Preparation Example 21

To a mixture of the intermediate compound 47 1.1 g, sodium acetate 1.2 g, THF 4 ml and water 2 mL was added 30% hydrogen peroxide aqueous solution 1.1 mL, and the mixture was stirred at 0° C. for 4 hours. Saturated aqueous solution of sodium thiosulfate 20 mL was added to the resulting mixture, and the mixture was stirred for 1 hour. Saturated aqueous solution of sodium hydrogen carbonate was added to the resulting mixture, and the mixture was extracted with chloroform. The resulting organic layer was dried over anhydrous sodium sulfate, and concentrated under reduced pressure to obtain the present compound 9 represented by the following formula 0.63 g.

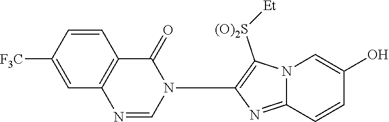

Present compound 9: ¹H-NMR (CDCl₃) δ: 8.56-8.47 (2H, m), 8.33-8.27 (1H, m), 8.11 (1H, s), 7.80 (1H, d), 7.71-7.64 (1H, m), 7.40-7.32 (1H, m), 3.50 (2H, q), 1.42 (3H, t).

### Preparation Example 22

A mixture of the present compound 9 30 mg, cesium carbonate 443 mg, ethyl p-toluene sulfonate 136 mg, and NMP 3 mL was stirred at 80° C. for 1 hour. Saturated aqueous solution of sodium thiosulfate 20 mL was added to the resulting mixture, and the mixture was stirred for 1 hour. The resulting mixture was stood to cool to room temperature, and water was then added thereto, and the mixture was extracted with ethyl acetate. The resulting organic layer was dried over anhydrous sodium sulfate, and concentrated under reduced pressure. The resulting residue was subjected to a silica gel column chromatography (ethylacetate:hexane=1:1) to obtain the present compound 10-1 represented by the following formula 100 mg.

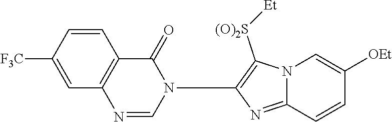

Present compound 10-1: ¹H-NMR (CDCl₃) δ: 8.51-8.46 (2H, m), 8.28 (1H, s), 8.11 (1H, s), 7.80 (1H, d), 7.72 (1H, d), 7.41-7.38 (1H, m), 4.14 (2H, q), 3.54 (2H, q), 1.54 (3H, t), 1.46 (3H, t).

### Preparation Example 23

To a mixture of the intermediate compound 43 150 mg and ethanol 5 ml were added methyl 2-formyl-5-(trifluoromethyl)benzoate which was prepared by the method described in Tetrahedron Letters, 2018, 59, 1564 164 mg and concentrated hydrochloric acid 18 μL under ice-cooling, and the mixture was stirred for 30 minutes under ice-cooling. The resulting mixture was concentrated under reduced pressure, and toluene 20 mL and para-toluenesulfonic acid 24 mg were added thereto, and the mixture was stirred at 110° C. for 6 hours. The resulting mixture was stood to cool to room temperature, and saturated aqueous solution of hydrogen bicarbonate was added thereto, and the mixture was extracted with ethyl acetate. The resulting organic layer was dried over anhydrous sodium sulfate, and concentrated under reduced pressure. The resulting residue was subjected to a silica gel column chromatography (hexane:ethylacetate=1:2) to obtain the present compound 11 represented by the following formula 155 mg.

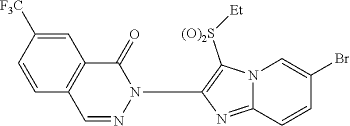

Present compound 11: ¹H-NMR (CDCl₃) δ: 9.05 (1H, d), 8.76 (1H, d), 8.40 (1H, s), 8.11 (1H, dd), 7.93 (1H, d), 7.72 (1H, d), 7.63 (1H, dd), 3.58 (2H, q), 1.46 (3H, t).

### Preparation Example 24

A mixture of the present compound 3-7 500 mg, pyrazole 73 mg, copper(I) iodide 19 mg, and N,N′-dimethylethylene diamine 19 mg, cesium carbonate 63 mg, and DMF 1 ml was stirred at 150° C. for 9 hours. Water was added to the resulting mixture, and the mixture was extracted with ethyl acetate. The resulting organic layer was dried over anhydrous sodium sulfate and concentrated under reduced pressure. The resulting residue was subjected to a silica gel column chromatography (hexane:ethylacetate: 1:2) to obtain the present compound 12-1 represented by the following formula 40 mg.

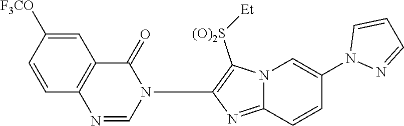

Present compound 12-1: ¹H-NMR (CDCl₃) δ: 9.33 (1H, d), 8.24 (1H, s), 8.17 (1H, d), 8.05 (1H, dd), 7.98 (1H, d), 7.91 (1H, d), 7.86 (1H, d), 7.83 (1H, d), 7.67 (1H, dd), 6.60 (1H, t), 3.61 (2H, q), 1.48 (3H, t).

The compound which was prepared according to the Preparation Example 24 and its physical property value was shown below.

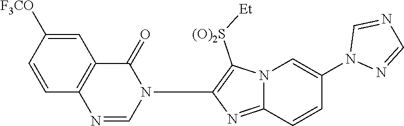

Present compound 12-2: ¹H-NMR (CDCl₃) δ: 9.40 (1H, s), 8.68 (1H, s), 8.26 (1H, s), 8.22 (1H, s), 8.18 (1H, d), 7.99-7.96 (2H, m), 7.87 (1H, d), 7.68 (1H, dd), 3.63 (2H, q), 1.49 (3H, t).

### Preparation Example 25

A mixture of the present compound 3-7 1.00 g, tert-butyl carbamate 272 mg, 2-dicyclohexylphosphino-2′,4′,6′-triisopropylbiphenyl 184 mg, palladium(II) acetate 69 mg, cesium carbonate 880 mg, and dioxane 15 ml was stirred at 100° C. under nitrogen atmosphere for 18 hours. Water was added to the resulting mixture at room temperature, and the mixture was extracted with ethyl acetate. The resulting organic layer was washed with saturated brine, dried over anhydrous sodium sulfate, and concentrated under reduced pressure. The resulting residue was subjected to a silica gel column chromatography (ethylacetate:hexane=1:1) to obtain the present compound 13 represented by the following formula 173 mg.

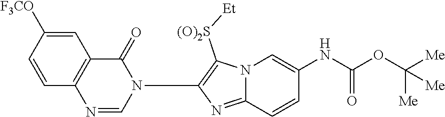

Present compound 13: ¹H-NMR (CDCl₃) δ: 9.31 (1H, s), 8.21 (1H, s), 8.17-8.15 (1H, m), 7.85 (1H, d), 7.72-7.70 (1H, m), 7.68-7.63 (2H, m), 6.64 (1H, s), 3.59-3.50 (2H, m), 1.58 (9H, s), 1.46 (3H, t).

Next, examples of the present compound X which was prepared according to any method of the Preparation Examples described in the Examples or the process described herein were described below. Here T1 to T36 represents any group indicated below.

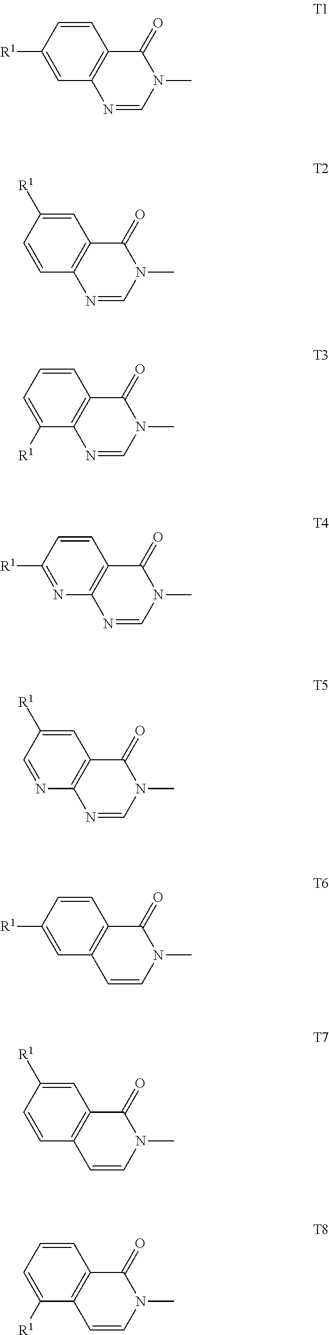

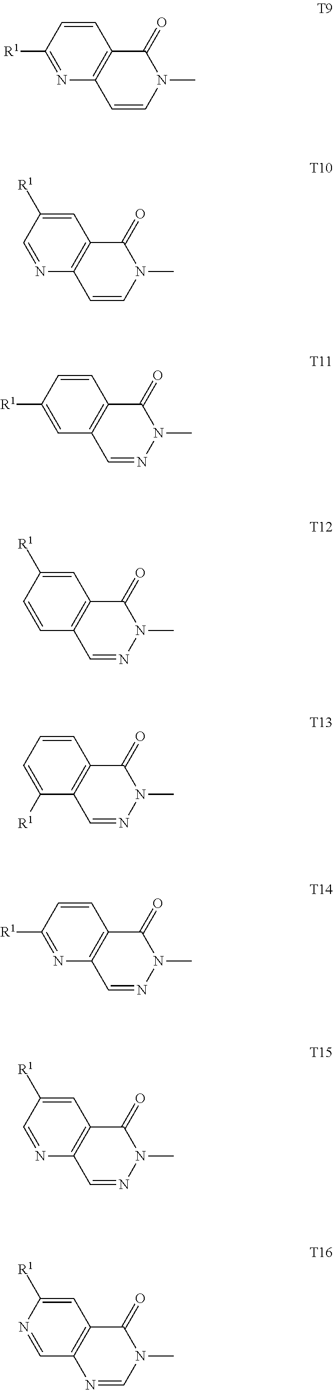

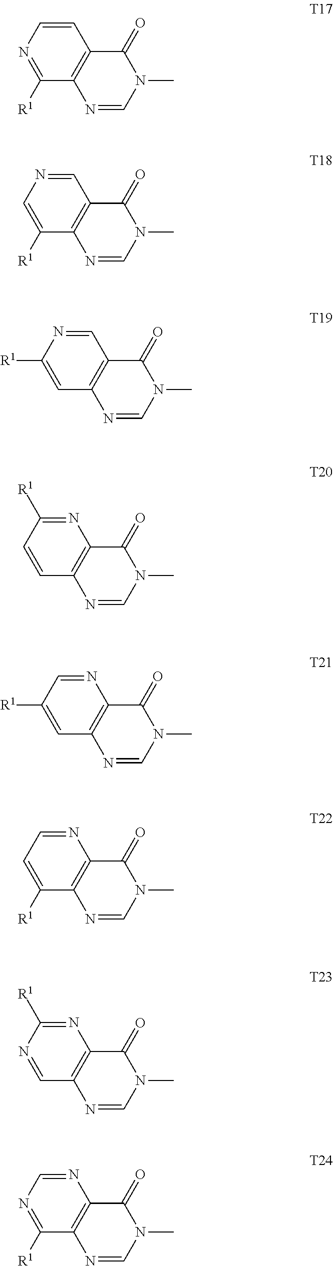

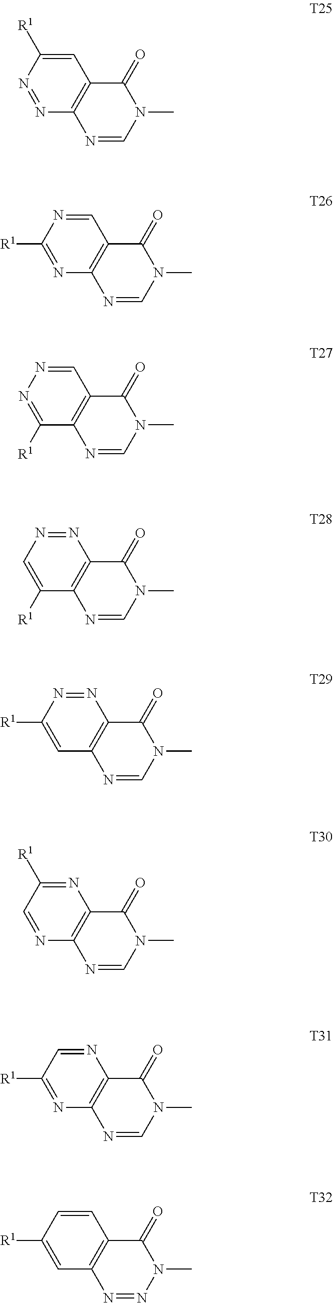

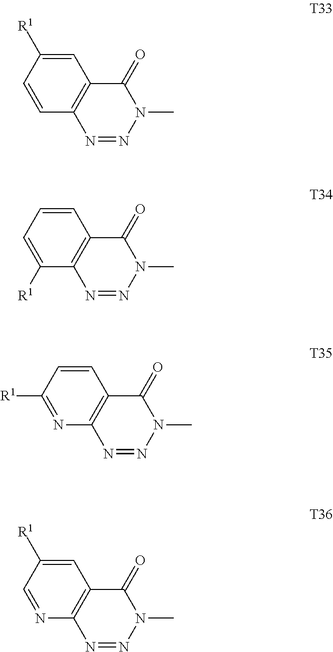

A compound represented by formula (L-1):

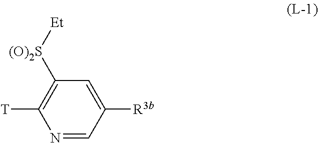

(hereinafter, referred to as compound (L-1)), wherein T represents a group represented by T1, R^{3b} represents a hydrogen atom, and R¹ represents any substituents indicated in [Table L1] to [Table L3](hereinafter, referred to as Compound Class SX₁).

TABLE L1

CF₃
CHF₂
CH₂C₃
CF₂CF₃
CH₂CF₂CF₃
CF₂CF₂CF₃
CF₂CF₂CF₂CF₃
CF₂CF₂CF₂CF₂CF₃
CF(CF₃)₂
C(Me)₂CN
CH₂C(Me)₂CN
CH₂CH₂C(Me)₂CN
c-Pr
c-Bu
1-CN-c-Pr
2,2-F₂-c-Pr
Oc-Pr
Oc-Bu
O(1-CN-c-Pr)
O(2,2-F₂-c-Pr)

TABLE L2

SCF₃
SCH₂CF₃
SCF₂CF₃
SCH₂CF₂CF₃
SCF₂CF₂CF₃
SCH₂CF₂CF₂CF₃
SCF₂CF₂CF₂CF₃
S(O)CF₃
S(O)CH₂CF₃
S(O)CF₂CF₃
S(O)CH₂CF₂CF₃
S(O)CF₂CF₂CF₃
S(O)CH₂CF₂CF₂CF₃
S(O)CF₂CF₂CF₂CF₃
S(O)₂CF₃
S(O)₂CH₂CF₃
S(O)₂CF₂CF₃
S(O)₂CH₂CF₂CF₃
S(O)₂CF₂CF₂CF₃
S(O)₂CH₂CF₂CF₂CF₃
S(O)₂CF₂CF₂CF₂CF₃

TABLE L3

OCF₃
OCHF₂
OCH₂CF₃
OCH₂CHF₂
OCF₂CF₃
OCH(CH₃)CF₃
OCH₂CF₂CHF₂
OCH₂CF₂CF₃
OCF₂CF₂CF₃
OCH₂CF₂CHFCF₃
OCH₂CF₂CF₂CF₃
OCF₂CF₂CF₂CF₃
OCH₂CF₂CF₂CF₂CF₃
OCH₂C(Me)₂CN
OCH₂CH₂C(Me)₂CN
OS(O)₂CF₃
OS(O)₂CF₂CF₃
OS(O)₂CF₂CF₂CF₃
F
Cl
Br
I

A compound (L-1) wherein T represents a group represented by T1, R^{3b} represents a chlorine atom, and R¹ represents any substituents indicated in [Table L1] to [Table L3](hereinafter, referred to as Compound Class SX₂).

A compound (L-1) wherein T represents a group represented by T1, R^{3b} represents a cyclopropyl group, and R¹ represents any substituents indicated in [Table L1] to [Table L3](hereinafter, referred to as Compound Class SX₃).

A compound (L-1) wherein T represents a group represented by T1, R^{3b} represents a 1-cyanocyclopropyl group, and R¹ represents any substituents indicated in [Table L1] to [Table L3](hereinafter, referred to as Compound Class SX₄).

A compound (L-1) wherein T represents a group represented by T1, R^{3b} represents CF₃, and R¹ represents any substituents indicated in [Table L1] to [Table L3](hereinafter, referred to as Compound Class SX₅).

A compound (L-1) wherein T represents a group represented by T2, R^{3b} represents a hydrogen atom, and R¹ represents any substituents indicated in [Table L1] to [Table L3](hereinafter, referred to as Compound Class SX₆).

A compound (L-1) wherein T represents a group represented by T2, R^{3b} represents a chlorine atom, and R¹ represents any substituents indicated in [Table L1] to [Table L3](hereinafter, referred to as Compound Class SX₇).

A compound (L-1) wherein T represents a group represented by T2, R^{3b} represents a cyclopropyl group, and R¹ represents any substituents indicated in [Table L1] to [Table L3](hereinafter, referred to as Compound Class SX₈).

A compound (L-1) wherein T represents a group represented by T2, R^{3b} represents a cyanocyclopropyl group, and R¹ represents any substituents indicated in [Table L1] to [Table L3](hereinafter, referred to as Compound Class SX₉).

A compound (L-1) wherein T represents a group represented by T2, R^{3b} represents CF₃, and R¹ represents any substituents indicated in [Table L1] to [Table L3](hereinafter, referred to as Compound Class SX₁₀).

A compound (L-1) wherein T represents a group represented by T3, R^{3b} represents a hydrogen atom, and R¹ represents any substituents indicated in [Table L1] to [Table L3](hereinafter, referred to as Compound Class SX₁₁).

A compound (L-1) wherein T represents a group represented by T3, R³¹ represents a chlorine atom, and R¹ represents any substituents indicated in [Table L1] to [Table L3](hereinafter, referred to as Compound Class SX₁₂).

A compound (L-1) wherein T represents a group represented by T3, R³¹ represents a cyclopropyl group, and R¹ represents any substituents indicated in [Table L1] to [Table L3](hereinafter, referred to as Compound Class SX₁₃).

A compound (L-1) wherein T represents a group represented by T3, R^{3b} represents a 1-cyanocyclopropyl group, and R¹ represents any substituents indicated in [Table L1] to [Table L3](hereinafter, referred to as Compound Class SX₁₄).

A compound (L-1) wherein T represents a group represented by T3, R^{3b} represents CF₃, and R¹ represents any substituents indicated in [Table L1] to [Table L3](hereinafter, referred to as Compound Class SX₁₅).

A compound (L-1) wherein T represents a group represented by T4, R³¹ represents a hydrogen atom, and R¹ represents any substituents indicated in [Table L1] to [Table L3](hereinafter, referred to as Compound Class SX₁₆).

A compound (L-1) wherein T represents a group represented by T4, R³¹ represents a chlorine atom, and R¹ represents any substituents indicated in [Table L1] to [Table L3](hereinafter, referred to as Compound Class SX₁₇).

A compound (L-1) wherein T represents a group represented by T4, R³¹ represents a cyclopropyl group, and R¹ represents any substituents indicated in [Table L1] to [Table L3](hereinafter, referred to as Compound Class SX₁₈).

A compound (L-1) wherein T represents a group represented by T4, R^{3b} represents a 1-cyanocyclopropyl group, and R¹ represents any substituents indicated in [Table L1] to [Table L3](hereinafter, referred to as Compound Class SX₁₉).

A compound (L-1) wherein T represents a group represented by T4, R^{3b} represents CF₃, and R¹ represents any substituents indicated in [Table L1] to [Table L3](hereinafter, referred to as Compound Class SX₂₀).

A compound (L-1) wherein T represents a group represented by T5, R^{3b} represents a hydrogen atom, and R¹ represents any substituents indicated in [Table L1] to [Table L3](hereinafter, referred to as Compound Class SX₂₁).

A compound (L-1) wherein T represents a group represented by T5, R³¹ represents a chlorine atom, and R¹ represents any substituents indicated in [Table L1] to [Table L3](hereinafter, referred to as Compound Class SX₂₂).

A compound (L-1) wherein T represents a group represented by T5, R³¹ represents a cyclopropyl group, and R¹ represents any substituents indicated in [Table L1] to [Table L3](hereinafter, referred to as Compound Class SX₂₃).

A compound (L-1) wherein T represents a group represented by T5, R^{3b} represents a 1-cyanocyclopropyl group, and R¹ represents any substituents indicated in [Table L1] to [Table L3](hereinafter, referred to as Compound Class SX₂₄).

A compound (L-1) wherein T represents a group represented by T5, R^{3b} represents CF₃, and R¹ represents any substituents indicated in [Table L1] to [Table L3](hereinafter, referred to as Compound Class SX₂₅).

A compound (L-1) wherein T represents a group represented by T6, R^{3b} represents a hydrogen atom, and R¹ represents any substituents indicated in [Table L1] to [Table L3](hereinafter, referred to as Compound Class SX₂₆).

A compound (L-1) wherein T represents a group represented by T6, R³¹ represents a chlorine atom, and R¹ represents any substituents indicated in [Table L1] to [Table L3](hereinafter, referred to as Compound Class SX₂₇).

A compound (L-1) wherein T represents a group represented by T6, R³¹ represents a cyclopropyl group, and R¹ represents any substituents indicated in [Table L1] to [Table L3](hereinafter, referred to as Compound Class SX₂₈).

A compound (L-1) wherein T represents a group represented by T6, R^{3b} represents a 1-cyanocyclopropyl group, and R¹ represents any substituents indicated in [Table L1] to [Table L3](hereinafter, referred to as Compound Class SX₂₉).

A compound (L-1) wherein T represents a group represented by T6, R^{3b} represents CF₃, and R¹ represents any substituents indicated in [Table L1] to [Table L3](hereinafter, referred to as Compound Class SX₃₀).

A compound (L-1) wherein T represents a group represented by T7, R³¹ represents a hydrogen atom, and R¹ represents any substituents indicated in [Table L1] to [Table L3](hereinafter, referred to as Compound Class SX₃₁).

A compound (L-1) wherein T represents a group represented by T7, R^{3b} represents a chlorine atom, and R¹ represents any substituents indicated in [Table L1] to [Table L3](hereinafter, referred to as Compound Class SX₃₂).

A compound (L-1) wherein T represents a group represented by T7, R³¹ represents a cyclopropyl group, and R¹ represents any substituents indicated in [Table L1] to [Table L3](hereinafter, referred to as Compound Class SX₃₃).

A compound (L-1) wherein T represents a group represented by T7, R^{3b} represents a 1-cyanocyclopropyl group, and R¹ represents any substituents indicated in [Table L1] to [Table L3](hereinafter, referred to as Compound Class SX₃₄).

A compound (L-1) wherein T represents a group represented by T7, R^{3b} represents CF₃, and R¹ represents any substituents indicated in [Table L1] to [Table L3](hereinafter, referred to as Compound Class SX₃₅).

A compound (L-1) wherein T represents a group represented by T8, R³¹ represents a hydrogen atom, and R¹ represents any substituents indicated in [Table L1] to [Table L3](hereinafter, referred to as Compound Class SX₃₆).

A compound (L-1) wherein T represents a group represented by T8, R³¹ represents a chlorine atom, and R¹ represents any substituents indicated in [Table L1] to [Table L3](hereinafter, referred to as Compound Class SX₃₇).

A compound (L-1) wherein T represents a group represented by T8, R^{3b} represents a cyclopropyl group, and R¹ represents any substituents indicated in [Table L1] to [Table L3](hereinafter, referred to as Compound Class SX₃₈).

A compound (L-1) wherein T represents a group represented by T8, R^{3b} represents a 1-cyanocyclopropyl group, and R¹ represents any substituents indicated in [Table L1] to [Table L3](hereinafter, referred to as Compound Class SX₃₉).

A compound (L-1) wherein T represents a group represented by T8, R^{3b} represents CF₃, and R¹ represents any substituents indicated in [Table L1] to [Table L3](hereinafter, referred to as Compound Class SX₄₀).

A compound (L-1) wherein T represents a group represented by T9, R³¹ represents a hydrogen atom, and R¹ represents any substituents indicated in [Table L1] to [Table L3](hereinafter, referred to as Compound Class SX₄₁).

A compound (L-1) wherein T represents a group represented by T9, R³¹ represents a chlorine atom, and R¹ represents any substituents indicated in [Table L1] to [Table L3](hereinafter, referred to as Compound Class SX₄₂).

A compound (L-1) wherein T represents a group represented by T9, R^{3b} represents a cyclopropyl group, and R¹ represents any substituents indicated in [Table L1] to [Table L3](hereinafter, referred to as Compound Class SX₄₃).

A compound (L-1) wherein T represents a group represented by T9, R^{3b} represents a 1-cyanocyclopropyl group, and R¹ represents any substituents indicated in [Table L1] to [Table L3](hereinafter, referred to as Compound Class SX₄₄).

A compound (L-1) wherein T represents a group represented by T9, R^{3b} represents CF₃, and R¹ represents any substituents indicated in [Table L1] to [Table L3](hereinafter, referred to as Compound Class SX₄₅).

A compound (L-1) wherein T represents a group represented by T10, R³¹ represents a hydrogen atom, and R¹ represents any substituents indicated in [Table L1] to [Table L3](hereinafter, referred to as Compound Class SX₄₆).

A compound (L-1) wherein T represents a group represented by T10, R³¹ represents a chlorine atom, and R¹ represents any substituents indicated in [Table L1] to [Table L3](hereinafter, referred to as Compound Class SX₄₇).

A compound (L-1) wherein T represents a group represented by T10, R^{3b} represents a cyclopropyl group, and R¹ represents any substituents indicated in [Table L1] to [Table L3](hereinafter, referred to as Compound Class SX₄₈).

A compound (L-1) wherein T represents a group represented by T10, R^{3b} represents a 1-cyanocyclopropyl group, and R¹ represents any substituents indicated in [Table L1] to [Table L3](hereinafter, referred to as Compound Class SX₄₉).

A compound (L-1) wherein T represents a group represented by T10, R^{3b} represents CF₃, and R¹ represents any substituents indicated in [Table L1] to [Table L3](hereinafter, referred to as Compound Class SX₅₀).

A compound (L-1) wherein T represents a group represented by T1l, R^{3b} represents a hydrogen atom, and R¹ represents any substituents indicated in [Table L1] to [Table L3](hereinafter, referred to as Compound Class SX₅₁).

A compound (L-1) wherein T represents a group represented by T1l, R^{3b} represents a chlorine atom, and R¹ represents any substituents indicated in [Table L1] to [Table L3](hereinafter, referred to as Compound Class SX₅₂).

A compound (L-1) wherein T represents a group represented by T1l, R^{3b} represents a cyclopropyl group, and R¹ represents any substituents indicated in [Table L1] to [Table L3](hereinafter, referred to as Compound Class SX₅₃).

A compound (L-1) wherein T represents a group represented by T1l, R^{3b} represents a 1-cyanocyclopropyl group, and R¹ represents any substituents indicated in [Table L1] to [Table L3](hereinafter, referred to as Compound Class SX₅₄).

A compound (L-1) wherein T represents a group represented by T1l, R^{3b} represents CF₃, and R¹ represents any substituents indicated in [Table L1] to [Table L3](hereinafter, referred to as Compound Class SX₅₅).

A compound (L-1) wherein T represents a group represented by T12, R^{3b} represents a hydrogen atom, and R¹ represents any substituents indicated in [Table L1] to [Table L3](hereinafter, referred to as Compound Class SX₅₆).

A compound (L-1) wherein T represents a group represented by T12, R^{3b} represents a chlorine atom, and R¹ represents any substituents indicated in [Table L1] to [Table L3](hereinafter, referred to as Compound Class SX₅₇).

A compound (L-1) wherein T represents a group represented by T12, R^{3b} represents a cyclopropyl group, and R¹ represents any substituents indicated in [Table L1] to [Table L3](hereinafter, referred to as Compound Class SX₅₈).

A compound (L-1) wherein T represents a group represented by T12, R^{3b} represents a 1-cyanocyclopropyl group, and R¹ represents any substituents indicated in [Table L1] to [Table L3](hereinafter, referred to as Compound Class SX₅₉).

A compound (L-1) wherein T represents a group represented by T12, R^{3b} represents CF₃, and R¹ represents any substituents indicated in [Table L1] to [Table L3](hereinafter, referred to as Compound Class SX₆₀).

A compound (L-1) wherein T represents a group represented by T13, R^{3b} represents a hydrogen atom, and R¹ represents any substituents indicated in [Table L1] to [Table L3](hereinafter, referred to as Compound Class SX₆₂).

A compound (L-1) wherein T represents a group represented by T13, R^{3b} represents a chlorine atom, and R¹ represents any substituents indicated in [Table L1] to [Table L3](hereinafter, referred to as Compound Class SX₆₂).

A compound (L-1) wherein T represents a group represented by T13, R^{3b} represents a cyclopropyl group, and R¹ represents any substituents indicated in [Table L1] to [Table L3](hereinafter, referred to as Compound Class SX₆₃).

A compound (L-1) wherein T represents a group represented by T13, R^{3b} represents a 1-cyanocyclopropyl group, and R¹ represents any substituents indicated in [Table L1] to [Table L3](hereinafter, referred to as Compound Class SX₆₄).

A compound (L-1) wherein T represents a group represented by T13, R^{3b} represents CF₃, and R¹ represents any substituents indicated in [Table L1] to [Table L3](hereinafter, referred to as Compound Class SX₆₅).

A compound (L-1) wherein T represents a group represented by T14, R^{3b} represents a hydrogen atom, and R¹ represents any substituents indicated in [Table L1] to [Table L3](hereinafter, referred to as Compound Class SX₆₆).

A compound (L-1) wherein T represents a group represented by T14, R^{3b} represents a chlorine atom, and R¹ represents any substituents indicated in [Table L1] to [Table L3](hereinafter, referred to as Compound Class SX₆₇).

A compound (L-1) wherein T represents a group represented by T14, R^{3b} represents a cyclopropyl group, and R¹ represents any substituents indicated in [Table L1] to [Table L3](hereinafter, referred to as Compound Class SX₆₈).

A compound (L-1) wherein T represents a group represented by T14, R^{3b} represents a 1-cyanocyclopropyl group, and R¹ represents any substituents indicated in [Table L1] to [Table L3](hereinafter, referred to as Compound Class SX₆₉).

A compound (L-1) wherein T represents a group represented by T14, R^{3b} represents CF₃, and R¹ represents any substituents indicated in [Table L1] to [Table L3](hereinafter, referred to as Compound Class SX₇₀).

A compound (L-1) wherein T represents a group represented by T15, R^{3b} represents a hydrogen atom, and R¹ represents any substituents indicated in [Table L1] to [Table L3](hereinafter, referred to as Compound Class SX₇₁).

A compound (L-1) wherein T represents a group represented by T15, R^{3b} represents a chlorine atom, and R¹ represents any substituents indicated in [Table L1] to [Table L3](hereinafter, referred to as Compound Class SX₇₂).

A compound (L-1) wherein T represents a group represented by T15, R^{3b} represents a cyclopropyl group, and R¹ represents any substituents indicated in [Table L1] to [Table L3](hereinafter, referred to as Compound Class SX₇₃).

A compound (L-1) wherein T represents a group represented by T15, R^{3b} represents a 1-cyanocyclopropyl group, and R¹ represents any substituents indicated in [Table L1] to [Table L3](hereinafter, referred to as Compound Class SX₇₄).

A compound (L-1) wherein T represents a group represented by T15, R^{3b} represents CF₃, and R¹ represents any substituents indicated in [Table L1] to [Table L3](hereinafter, referred to as Compound Class SX₇₅).

A compound represented by formula (L-2):

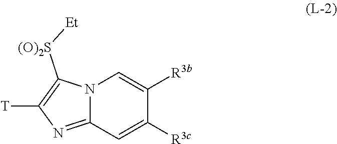

(hereinafter, referred to as compound (L-2)), wherein T represents a group represented by T1, R^{3b} represents a hydrogen atom, R^{3c} represents a hydrogen atom, and R¹ represents any substituents indicated in [Table L1] to [Table L3](hereinafter, referred to as Compound Class SX₇₆).

A compound (L-2) wherein T represents a group represented by T1, R^{3b} represents CF₃, R^{3c} represents a hydrogen atom, and R¹ represents any substituents indicated in [Table L1] to [Table L3](hereinafter, referred to as Compound Class SX₇₇).

A compound (L-2) wherein T represents a group represented by T1, R^{3b} represents a chlorine atom, R^{3c} represents a hydrogen atom, and R¹ represents any substituents indicated in [Table L1] to [Table L3](hereinafter, referred to as Compound Class SX₇₈).

A compound (L-2) wherein T represents a group represented by T1, R^{3b} represents a bromine atom, R^{3c} represents a hydrogen atom, and R¹ represents any substituents indicated in [Table L1] to [Table L3](hereinafter, referred to as Compound Class SX₇₉).

A compound (L-2) wherein T represents a group represented by T1, R^{3b} represents an iodine atom, R^{3c} represents a hydrogen atom, and R¹ represents any substituents indicated in [Table L1] to [Table L3](hereinafter, referred to as Compound Class SX₈₀).

A compound (L-2) wherein T represents a group represented by T1, R^{3b} represents a hydrogen atom, R^{3c} represents CF₃, and R¹ represents any substituents indicated in [Table L1] to [Table L3](hereinafter, referred to as Compound Class SX₈₁).

A compound (L-2) wherein T represents a group represented by T1, R^{3b} represents a hydrogen atom, R^{3c} represents a chlorine atom, and R¹ represents any substituents indicated in [Table L1] to [Table L3](hereinafter, referred to as Compound Class SX₈₂).

A compound (L-2) wherein T represents a group represented by T1, R^{3b} represents a hydrogen atom, R^{3c} represents a bromine atom and R¹ represents any substituents indicated in [Table L1] to [Table L3](hereinafter, referred to as Compound Class SX₈₃).

A compound (L-2) wherein T represents a group represented by T1, R^{3b} represents a hydrogen atom, R^{3c} represents an iodine atom and R¹ represents any substituents indicated in [Table L1] to [Table L3](hereinafter, referred to as Compound Class SX₈₄).

A compound (L-2) wherein T represents a group represented by T2, R^{3b} represents a hydrogen atom, R^{3c} represents a hydrogen atom and R¹ represents any substituents indicated in [Table L1] to [Table L3](hereinafter, referred to as Compound Class SX₈₅).

A compound (L-2) wherein T represents a group represented by T2, R^{3b} represents CF₃, R^{3c} represents a hydrogen atom, and R¹ represents any substituents indicated in [Table L1] to [Table L3](hereinafter, referred to as Compound Class SX₈₆).

A compound (L-2) wherein T represents a group represented by T2, R^{3b} represents a chlorine atom, R^{3c} represents a hydrogen atom, and R¹ represents any substituents indicated in [Table L1] to [Table L3](hereinafter, referred to as Compound Class SX₈₇).

A compound (L-2) wherein T represents a group represented by T2, R^{3b} represents a bromine atom, R^{3c} represents a hydrogen atom, and R¹ represents any substituents indicated in [Table L1] to [Table L3](hereinafter, referred to as Compound Class SX₈₈).

A compound (L-2) wherein T represents a group represented by T2, R^{3b} represents an iodine atom, R^{3c} represents a hydrogen atom, and R¹ represents any substituents indicated in [Table L1] to [Table L3](hereinafter, referred to as Compound Class SX₈₉).

A compound (L-2) wherein T represents a group represented by T2, R^{3b} represents a hydrogen atom, R^{3c} represents CF₃, and R¹ represents any substituents indicated in [Table L1] to [Table L3](hereinafter, referred to as Compound Class SX₉₀).

A compound (L-2) wherein T represents a group represented by T2, R^{3b} represents a hydrogen atom, R^{3c} represents a chlorine atom, and R¹ represents any substituents indicated in [Table L1] to [Table L3](hereinafter, referred to as Compound Class SX₉₁).

A compound (L-2) wherein T represents a group represented by T2, R^{3b} represents a hydrogen atom, R^{3c} represents a bromine atom and R¹ represents any substituents indicated in [Table L1] to [Table L3](hereinafter, referred to as Compound Class SX₉₂).

A compound (L-2) wherein T represents a group represented by T2, R^{3b} represents a hydrogen atom, R^{3c} represents an iodine atom and R¹ represents any substituents indicated in [Table L1] to [Table L3](hereinafter, referred to as Compound Class SX₉₃).

A compound (L-2) wherein T represents a group represented by T3, R^{3b} represents a hydrogen atom, R^{3c} represents a hydrogen atom and R¹ represents any substituents indicated in [Table L1] to [Table L3](hereinafter, referred to as Compound Class SX₉₄).

A compound (L-2) wherein T represents a group represented by T3, R^{3b} represents CF₃, R^{3c} represents a hydrogen atom, and R¹ represents any substituents indicated in [Table L1] to [Table L3](hereinafter, referred to as Compound Class SX₉₅).

A compound (L-2) wherein T represents a group represented by T3, R^{3b} represents a chlorine atom, R^{3c} represents a hydrogen atom, and R¹ represents any substituents indicated in [Table L1] to [Table L3](hereinafter, referred to as Compound Class SX₉₆).

A compound (L-2) wherein T represents a group represented by T3, R^{3b} represents a bromine atom, R^{3c} represents a hydrogen atom, and R¹ represents any substituents indicated in [Table L1] to [Table L3](hereinafter, referred to as Compound Class SX₉₇).

A compound (L-2) wherein T represents a group represented by T3, R^{3b} represents an iodine atom, R^{3c} represents a hydrogen atom, and R¹ represents any substituents indicated in [Table L1] to [Table L3](hereinafter, referred to as Compound Class SX₉₈).

A compound (L-2) wherein T represents a group represented by T3, R^{3b} represents a hydrogen atom, R^{3c} represents CF₃, and R¹ represents any substituents indicated in [Table L1] to [Table L3](hereinafter, referred to as Compound Class SX₉₉).

A compound (L-2) wherein T represents a group represented by T3, R^{3b} represents a hydrogen atom, R^{3c} represents a chlorine atom, and R¹ represents any substituents indicated in [Table L1] to [Table L3](hereinafter, referred to as Compound Class SX₁₀₀).

A compound (L-2) wherein T represents a group represented by T3, R^{3b} represents a hydrogen atom, R^{3c} represents a bromine atom and R¹ represents any substituents indicated in [Table L1] to [Table L3](hereinafter, referred to as Compound Class SX₁₀₁).

A compound (L-2) wherein T represents a group represented by T3, R^{3b} represents a hydrogen atom, R^{3c} represents an iodine atom and R¹ represents any substituents indicated in [Table L1] to [Table L3](hereinafter, referred to as Compound Class SX₁₀₂).

A compound (L-2) wherein T represents a group represented by T4, R^{3b} represents a hydrogen atom, R^{3c} represents a hydrogen atom and R¹ represents any substituents indicated in [Table L1] to [Table L3](hereinafter, referred to as Compound Class SX₁₀₃).

A compound (L-2) wherein T represents a group represented by T4, R^{3b} represents CF₃, R^{3c} represents a hydrogen atom, and R¹ represents any substituents indicated in [Table L1] to [Table L3](hereinafter, referred to as Compound Class SX₁₀₄).

A compound (L-2) wherein T represents a group represented by T4, R^{3b} represents a chlorine atom, R^{3c} represents a hydrogen atom, and R¹ represents any substituents indicated in [Table L1] to [Table L3](hereinafter, referred to as Compound Class SX₁₀₅).

A compound (L-2) wherein T represents a group represented by T4, R^{3b} represents a bromine atom, R^{3c} represents a hydrogen atom, and R¹ represents any substituents indicated in [Table L1] to [Table L3](hereinafter, referred to as Compound Class SX₁₀₆).

A compound (L-2) wherein T represents a group represented by T4, R^{3b} represents an iodine atom, R^{3c} represents a hydrogen atom, and R¹ represents any substituents indicated in [Table L1] to [Table L3](hereinafter, referred to as Compound Class SX₁₀₇).

A compound (L-2) wherein T represents a group represented by T4, R^{3b} represents a hydrogen atom, R^{3c} represents CF₃, and R¹ represents any substituents indicated in [Table L1] to [Table L3](hereinafter, referred to as Compound Class SX₁₀₈).

A compound (L-2) wherein T represents a group represented by T4, R^{3b} represents a hydrogen atom, R^{3c} represents a chlorine atom, and R¹ represents any substituents indicated in [Table L1] to [Table L3](hereinafter, referred to as Compound Class SX₁₀₉).

A compound (L-2) wherein T represents a group represented by T4, R^{3b} represents a hydrogen atom, R^{3c} represents a bromine atom and R¹ represents any substituents indicated in [Table L1] to [Table L3](hereinafter, referred to as Compound Class SX₁₁₀).

A compound (L-2) wherein T represents a group represented by T4, R^{3b} represents a hydrogen atom, R^{3c} represents an iodine atom and R¹ represents any substituents indicated in [Table L1] to [Table L3](hereinafter, referred to as Compound Class SX₁₁₁).

A compound (L-2) wherein T represents a group represented by T5, R^{3b} represents a hydrogen atom, R^{3c} represents a hydrogen atom and R¹ represents any substituents indicated in [Table L1] to [Table L3](hereinafter, referred to as Compound Class SX₁₁₂).

A compound (L-2) wherein T represents a group represented by T5, R^{3b} represents CF₃, R^{3c} represents a hydrogen atom, and R¹ represents any substituents indicated in [Table L1] to [Table L3](hereinafter, referred to as Compound Class SX₁₁₃).

A compound (L-2) wherein T represents a group represented by T5, R^{3b} represents a chlorine atom, R^{3c} represents a hydrogen atom, and R¹ represents any substituents indicated in [Table L1] to [Table L3](hereinafter, referred to as Compound Class SX₁₁₄).

A compound (L-2) wherein T represents a group represented by T5, R^{3b} represents a bromine atom, R^{3c} represents a hydrogen atom, and R¹ represents any substituents indicated in [Table L1] to [Table L3](hereinafter, referred to as Compound Class SX₁₁₅).

A compound (L-2) wherein T represents a group represented by T5, R^{3b} represents an iodine atom, R^{3c} represents a hydrogen atom, and R¹ represents any substituents indicated in [Table L1] to [Table L3](hereinafter, referred to as Compound Class SX₁₁₆).

A compound (L-2) wherein T represents a group represented by T5, R^{3b} represents a hydrogen atom, R^{3c} represents CF₃, and R¹ represents any substituents indicated in [Table L1] to [Table L3](hereinafter, referred to as Compound Class SX₁₁₇).

A compound (L-2) wherein T represents a group represented by T5, R^{3b} represents a hydrogen atom, R^{3c} represents a chlorine atom, and R¹ represents any substituents indicated in [Table L1] to [Table L3](hereinafter, referred to as Compound Class SX₁₁₈).

A compound (L-2) wherein T represents a group represented by T5, R^{3b} represents a hydrogen atom, R^{3c} represents a bromine atom and R¹ represents any substituents indicated in [Table L1] to [Table L3](hereinafter, referred to as Compound Class SX₁₁₉).

A compound (L-2) wherein T represents a group represented by T5, R^{3b} represents a hydrogen atom, R^{3c} represents an iodine atom and R¹ represents any substituents indicated in [Table L1] to [Table L3](hereinafter, referred to as Compound Class SX₁₂₀).

A compound (L-2) wherein T represents a group represented by T6, R^{3b} represents a hydrogen atom, R^{3c} represents a hydrogen atom and R¹ represents any substituents indicated in [Table L1] to [Table L3](hereinafter, referred to as Compound Class SX₁₂₁).

A compound (L-2) wherein T represents a group represented by T6, R^{3b} represents CF₃, R^{3c} represents a hydrogen atom, and R¹ represents any substituents indicated in [Table L1] to [Table L3](hereinafter, referred to as Compound Class SX₁₂₂).

A compound (L-2) wherein T represents a group represented by T6, R^{3b} represents a chlorine atom, R^{3c} represents a hydrogen atom, and R¹ represents any substituents indicated in [Table L1] to [Table L3](hereinafter, referred to as Compound Class SX₁₂₃).

A compound (L-2) wherein T represents a group represented by T6, R^{3b} represents a bromine atom, R^{3c} represents a hydrogen atom, and R¹ represents any substituents indicated in [Table L1] to [Table L3](hereinafter, referred to as Compound Class SX₁₂₄).

A compound (L-2) wherein T represents a group represented by T6, R^{3b} represents an iodine atom, R^{3c} represents a hydrogen atom, and R¹ represents any substituents indicated in [Table L1] to [Table L3](hereinafter, referred to as Compound Class SX₁₂₅).

A compound (L-2) wherein T represents a group represented by T6, R^{3b} represents a hydrogen atom, R^{3c} represents CF₃, and R¹ represents any substituents indicated in [Table L1] to [Table L3](hereinafter, referred to as Compound Class SX₁₂₆).

A compound (L-2) wherein T represents a group represented by T6, R^{3b} represents a hydrogen atom, R^{3c} represents a chlorine atom, and R¹ represents any substituents indicated in [Table L1] to [Table L3](hereinafter, referred to as Compound Class SX₁₂₇).

A compound (L-2) wherein T represents a group represented by T6, R^{3b} represents a hydrogen atom, R^{3c} represents a bromine atom and R¹ represents any substituents indicated in [Table L1] to [Table L3](hereinafter, referred to as Compound Class SX₁₂₈).

A compound (L-2) wherein T represents a group represented by T6, R^{3b} represents a hydrogen atom, R^{3c} represents an iodine atom and R¹ represents any substituents indicated in [Table L1] to [Table L3](hereinafter, referred to as Compound Class SX₁₂₉).

A compound (L-2) wherein T represents a group represented by T7, R^{3b} represents a hydrogen atom, R^{3c} represents a hydrogen atom and R¹ represents any substituents indicated in [Table L1] to [Table L3](hereinafter, referred to as Compound Class SX₁₃₀).

A compound (L-2) wherein T represents a group represented by T7, R^{3b} represents CF₃, R^{3c} represents a hydrogen atom, and R¹ represents any substituents indicated in [Table L1] to [Table L3](hereinafter, referred to as Compound Class SX₁₃₁).

A compound (L-2) wherein T represents a group represented by T7, R^{3b} represents a chlorine atom, R^{3c} represents a hydrogen atom, and R¹ represents any substituents indicated in [Table L1] to [Table L3](hereinafter, referred to as Compound Class SX₁₃₂).

A compound (L-2) wherein T represents a group represented by T7, R^{3b} represents a bromine atom, R^{3c} represents a hydrogen atom, and R¹ represents any substituents indicated in [Table L1] to [Table L3](hereinafter, referred to as Compound Class SX₁₃₃).

A compound (L-2) wherein T represents a group represented by T7, R^{3b} represents an iodine atom, R^{3c} represents a hydrogen atom, and R¹ represents any substituents indicated in [Table L1] to [Table L3](hereinafter, referred to as Compound Class SX₁₃₄).

A compound (L-2) wherein T represents a group represented by T7, R^{3b} represents a hydrogen atom, R^{3c} represents CF₃, and R¹ represents any substituents indicated in [Table L1] to [Table L3](hereinafter, referred to as Compound Class SX₁₃₅).

A compound (L-2) wherein T represents a group represented by T7, R^{3b} represents a hydrogen atom, R^{3c} represents a chlorine atom, and R¹ represents any substituents indicated in [Table L1] to [Table L3](hereinafter, referred to as Compound Class SX₁₃₆).

A compound (L-2) wherein T represents a group represented by T7, R^{3b} represents a hydrogen atom, R^{3c} represents a bromine atom and R¹ represents any substituents indicated in [Table L1] to [Table L3](hereinafter, referred to as Compound Class SX₁₃₇).

A compound (L-2) wherein T represents a group represented by T7, R^{3b} represents a hydrogen atom, R^{3c} represents an iodine atom and R¹ represents any substituents indicated in [Table L1] to [Table L3](hereinafter, referred to as Compound Class SX₁₃₈).

A compound (L-2) wherein T represents a group represented by T8, R^{3b} represents a hydrogen atom, R^{3c} represents a hydrogen atom and R¹ represents any substituents indicated in [Table L1] to [Table L3](hereinafter, referred to as Compound Class SX₁₃₉).

A compound (L-2) wherein T represents a group represented by T8, R^{3b} represents CF₃, R^{3c} represents a hydrogen atom, and R¹ represents any substituents indicated in [Table L1] to [Table L3](hereinafter, referred to as Compound Class SX₁₄₀).

A compound (L-2) wherein T represents a group represented by T8, R^{3b} represents a chlorine atom, R^{3c} represents a hydrogen atom, and R¹ represents any substituents indicated in [Table L1] to [Table L3](hereinafter, referred to as Compound Class SX₁₄₁).

A compound (L-2) wherein T represents a group represented by T8, R^{3b} represents a bromine atom, R^{3c} represents a hydrogen atom, and R¹ represents any substituents indicated in [Table L1] to [Table L3](hereinafter, referred to as Compound Class SX₁₄₂).

A compound (L-2) wherein T represents a group represented by T8, R^{3b} represents an iodine atom, R^{3c} represents a hydrogen atom, and R¹ represents any substituents indicated in [Table L1] to [Table L3](hereinafter, referred to as Compound Class SX₁₄₃).

A compound (L-2) wherein T represents a group represented by T8, R^{3b} represents a hydrogen atom, R^{3c} represents CF₃, and R¹ represents any substituents indicated in [Table L1] to [Table L3](hereinafter, referred to as Compound Class SX₁₄₄).

A compound (L-2) wherein T represents a group represented by T8, R^{3b} represents a hydrogen atom, R^{3c} represents a chlorine atom, and R¹ represents any substituents indicated in [Table L1] to [Table L3](hereinafter, referred to as Compound Class SX₁₄₅).

A compound (L-2) wherein T represents a group represented by T8, R^{3b} represents a hydrogen atom, R^{3c} represents a bromine atom and R¹ represents any substituents indicated in [Table L1] to [Table L3](hereinafter, referred to as Compound Class SX₁₄₆).

A compound (L-2) wherein T represents a group represented by T8, R^{3b} represents a hydrogen atom, R^{3c} represents an iodine atom and R¹ represents any substituents indicated in [Table L1] to [Table L3](hereinafter, referred to as Compound Class SX₁₄₇).

A compound (L-2) wherein T represents a group represented by T9, R^{3b} represents a hydrogen atom, R^{3c} represents a hydrogen atom and R¹ represents any substituents indicated in [Table L1] to [Table L3](hereinafter, referred to as Compound Class SX₁₄₈).

A compound (L-2) wherein T represents a group represented by T9, R^{3b} represents CF₃, R^{3c} represents a hydrogen atom, and R¹ represents any substituents indicated in [Table L1] to [Table L3](hereinafter, referred to as Compound Class SX₁₄₉).

A compound (L-2) wherein T represents a group represented by T9, R^{3b} represents a chlorine atom, R^{3c} represents a hydrogen atom, and R¹ represents any substituents indicated in [Table L1] to [Table L3](hereinafter, referred to as Compound Class SX₁₅₀).

A compound (L-2) wherein T represents a group represented by T9, R^{3b} represents a bromine atom, R^{3c} represents a hydrogen atom, and R¹ represents any substituents indicated in [Table L1] to [Table L3](hereinafter, referred to as Compound Class SX₁₅₁).

A compound (L-2) wherein T represents a group represented by T9, R^{3b} represents an iodine atom, R^{3c} represents a hydrogen atom, and R¹ represents any substituents indicated in [Table L1] to [Table L3](hereinafter, referred to as Compound Class SX₁₅₂).

A compound (L-2) wherein T represents a group represented by T9, R^{3b} represents a hydrogen atom, R^{3c} represents CF₃, and R¹ represents any substituents indicated in [Table L1] to [Table L3](hereinafter, referred to as Compound Class SX₁₅₃).

A compound (L-2) wherein T represents a group represented by T9, R^{3b} represents a hydrogen atom, R^{3c} represents a chlorine atom, and R¹ represents any substituents indicated in [Table L1] to [Table L3](hereinafter, referred to as Compound Class SX₁₅₄).

A compound (L-2) wherein T represents a group represented by T9, R^{3b} represents a hydrogen atom, R^{3c} represents a bromine atom and R¹ represents any substituents indicated in [Table L1] to [Table L3](hereinafter, referred to as Compound Class SX₁₅₅).

A compound (L-2) wherein T represents a group represented by T9, R^{3b} represents a hydrogen atom, R^{3c} represents an iodine atom and R¹ represents any substituents indicated in [Table L1] to [Table L3](hereinafter, referred to as Compound Class SX₁₅₆).

A compound (L-2) wherein T represents a group represented by T10, R^{3b} represents a hydrogen atom, R^{3c} represents a hydrogen atom and R¹ represents any substituents indicated in [Table L1] to [Table L3](hereinafter, referred to as Compound Class SX₁₅₇).

A compound (L-2) wherein T represents a group represented by T10, R^{3b} represents CF₃, R^{3c} represents a hydrogen atom, and R¹ represents any substituents indicated in [Table L1] to [Table L3](hereinafter, referred to as Compound Class SX₁₅₈).

A compound (L-2) wherein T represents a group represented by T10, R^{3b} represents a chlorine atom, R^{3c} represents a hydrogen atom, and R¹ represents any substituents indicated in [Table L1] to [Table L3](hereinafter, referred to as Compound Class SX₁₅₉).

A compound (L-2) wherein T represents a group represented by T10, R^{3b} represents a bromine atom, R^{3c} represents a hydrogen atom, and R¹ represents any substituents indicated in [Table L1] to [Table L3](hereinafter, referred to as Compound Class SX₁₆₀).

A compound (L-2) wherein T represents a group represented by T10, R^{3b} represents an iodine atom, R^{3c} represents a hydrogen atom, and R¹ represents any substituents indicated in [Table L1] to [Table L3](hereinafter, referred to as Compound Class SX₁₆₁).

A compound (L-2) wherein T represents a group represented by T10, R^{3b} represents a hydrogen atom, R^{3c} represents CF₃, and R¹ represents any substituents indicated in [Table L1] to [Table L3](hereinafter, referred to as Compound Class SX₁₆₂).

A compound (L-2) wherein T represents a group represented by T10, R^{3b} represents a hydrogen atom, R^{3c} represents a chlorine atom, and R¹ represents any substituents indicated in [Table L1] to [Table L3](hereinafter, referred to as Compound Class SX₁₆₃).

A compound (L-2) wherein T represents a group represented by T10, R^{3b} represents a hydrogen atom, R^{3c} represents a bromine atom and R¹ represents any substituents indicated in [Table L1] to [Table L3](hereinafter, referred to as Compound Class SX₁₆₄).

A compound (L-2) wherein T represents a group represented by T10, R^{3b} represents a hydrogen atom, R^{3c} represents an iodine atom and R¹ represents any substituents indicated in [Table L1] to [Table L3](hereinafter, referred to as Compound Class SX₁₆₅).

A compound (L-2) wherein T represents a group represented by T1l, R^{3b} represents a hydrogen atom, R^{3c} represents a hydrogen atom and R¹ represents any substituents indicated in [Table L1] to [Table L3](hereinafter, referred to as Compound Class SX₁₆₆).

A compound (L-2) wherein T represents a group represented by T1l, R^{3b} represents CF₃, R^{3c} represents a hydrogen atom, and R¹ represents any substituents indicated in [Table L1] to [Table L3](hereinafter, referred to as Compound Class SX₁₆₇).

A compound (L-2) wherein T represents a group represented by T1l, R^{3b} represents a chlorine atom, R^{3c} represents a hydrogen atom, and R¹ represents any substituents indicated in [Table L1] to [Table L3](hereinafter, referred to as Compound Class SX₁₆₈).

A compound (L-2) wherein T represents a group represented by T1l, R^{3b} represents a bromine atom, R^{3c} represents a hydrogen atom, and R¹ represents any substituents indicated in [Table L1] to [Table L3](hereinafter, referred to as Compound Class SX₁₆₉).

A compound (L-2) wherein T represents a group represented by T1l, R^{3b} represents an iodine atom, R^{3c} represents a hydrogen atom, and R¹ represents any substituents indicated in [Table L1] to [Table L3](hereinafter, referred to as Compound Class SX₁₇₀).

A compound (L-2) wherein T represents a group represented by T1l, R^{3b} represents a hydrogen atom, R^{3c} represents CF₃, and R¹ represents any substituents indicated in [Table L1] to [Table L3](hereinafter, referred to as Compound Class SX₁₇₁).

A compound (L-2) wherein T represents a group represented by T1l, R^{3b} represents a hydrogen atom, R^{3c} represents a chlorine atom, and R¹ represents any substituents indicated in [Table L1] to [Table L3](hereinafter, referred to as Compound Class SX₁₇₂).

A compound (L-2) wherein T represents a group represented by T1l, R^{3b} represents a hydrogen atom, R^{3c} represents a bromine atom and R¹ represents any substituents indicated in [Table L1] to [Table L3](hereinafter, referred to as Compound Class SX₁₇₃).

A compound (L-2) wherein T represents a group represented by T1l, R^{3b} represents a hydrogen atom, R^{3c} represents an iodine atom and R¹ represents any substituents indicated in [Table L1] to [Table L3](hereinafter, referred to as Compound Class SX₁₇₄).

A compound (L-2) wherein T represents a group represented by T12, R^{3b} represents a hydrogen atom, R^{3c} represents a hydrogen atom and R¹ represents any substituents indicated in [Table L1] to [Table L3](hereinafter, referred to as Compound Class SX₁₇₅).

A compound (L-2) wherein T represents a group represented by T12, R^{3b} represents CF₃, R^{3c} represents a hydrogen atom, and R¹ represents any substituents indicated in [Table L1] to [Table L3](hereinafter, referred to as Compound Class SX₁₇₆).

A compound (L-2) wherein T represents a group represented by T12, R^{3b} represents a chlorine atom, R^{3c} represents a hydrogen atom, and R¹ represents any substituents indicated in [Table L1] to [Table L3](hereinafter, referred to as Compound Class SX₁₇₇).

A compound (L-2) wherein T represents a group represented by T12, R^{3b} represents a bromine atom, R^{3c} represents a hydrogen atom, and R¹ represents any substituents indicated in [Table L1] to [Table L3](hereinafter, referred to as Compound Class SX₁₇₈).

A compound (L-2) wherein T represents a group represented by T12, R^{3b} represents an iodine atom, R^{3c} represents a hydrogen atom, and R¹ represents any substituents indicated in [Table L1] to [Table L3](hereinafter, referred to as Compound Class SX₁₇₉).

A compound (L-2) wherein T represents a group represented by T12, R^{3b} represents a hydrogen atom, R^{3c} represents CF₃, and R¹ represents any substituents indicated in [Table L1] to [Table L3](hereinafter, referred to as Compound Class SX₁₈₀).

A compound (L-2) wherein T represents a group represented by T12, R^{3b} represents a hydrogen atom, R^{3c} represents a chlorine atom, and R¹ represents any substituents indicated in [Table L1] to [Table L3](hereinafter, referred to as Compound Class SX₁₈₁).

A compound (L-2) wherein T represents a group represented by T12, R^{3b} represents a hydrogen atom, R^{3c} represents a bromine atom and R¹ represents any substituents indicated in [Table L1] to [Table L3](hereinafter, referred to as Compound Class SX₁₈₂).

A compound (L-2) wherein T represents a group represented by T12, R^{3b} represents a hydrogen atom, R^{3c} represents an iodine atom and R¹ represents any substituents indicated in [Table L1] to [Table L3](hereinafter, referred to as Compound Class SX₁₈₃).

A compound (L-2) wherein T represents a group represented by T13, R^{3b} represents a hydrogen atom, R^{3c} represents a hydrogen atom and R¹ represents any substituents indicated in [Table L1] to [Table L3](hereinafter, referred to as Compound Class SX₁₈₄).

A compound (L-2) wherein T represents a group represented by T13, R^{3b} represents CF₃, R^{3c} represents a hydrogen atom, and R¹ represents any substituents indicated in [Table L1] to [Table L3](hereinafter, referred to as Compound Class SX₁₈₅).

A compound (L-2) wherein T represents a group represented by T13, R^{3b} represents a chlorine atom, R^{3c} represents a hydrogen atom, and R¹ represents any substituents indicated in [Table L1] to [Table L3](hereinafter, referred to as Compound Class SX₁₈₆).

A compound (L-2) wherein T represents a group represented by T13, R^{3b} represents a bromine atom, R^{3c} represents a hydrogen atom, and R¹ represents any substituents indicated in [Table L1] to [Table L3](hereinafter, referred to as Compound Class SX₁₈₇).

A compound (L-2) wherein T represents a group represented by T13, R^{3b} represents an iodine atom, R^{3c} represents a hydrogen atom, and R¹ represents any substituents indicated in [Table L1] to [Table L3](hereinafter, referred to as Compound Class SX₁₈₈).

A compound (L-2) wherein T represents a group represented by T13, R^{3b} represents a hydrogen atom, R^{3c} represents CF₃, and R¹ represents any substituents indicated in [Table L1] to [Table L3](hereinafter, referred to as Compound Class SX₁₈₉).

A compound (L-2) wherein T represents a group represented by T13, R^{3b} represents a hydrogen atom, R^{3c} represents a chlorine atom, and R¹ represents any substituents indicated in [Table L1] to [Table L3](hereinafter, referred to as Compound Class SX₁₉₀).

A compound (L-2) wherein T represents a group represented by T13, R^{3b} represents a hydrogen atom, R^{3c} represents a bromine atom and R¹ represents any substituents indicated in [Table L1] to [Table L3](hereinafter, referred to as Compound Class SX₁₉₁).

A compound (L-2) wherein T represents a group represented by T13, R^{3b} represents a hydrogen atom, R^{3c} represents an iodine atom and R¹ represents any substituents indicated in [Table L1] to [Table L3](hereinafter, referred to as Compound Class SX₁₉₂).

A compound (L-2) wherein T represents a group represented by T14, R^{3b} represents a hydrogen atom, R^{3c} represents a hydrogen atom and R¹ represents any substituents indicated in [Table L1] to [Table L3](hereinafter, referred to as Compound Class SX₁₉₃).

A compound (L-2) wherein T represents a group represented by T14, R^{3b} represents CF₃, R^{3c} represents a hydrogen atom, and R¹ represents any substituents indicated in [Table L1] to [Table L3](hereinafter, referred to as Compound Class SX₁₉₄).

A compound (L-2) wherein T represents a group represented by T14, R^{3b} represents a chlorine atom, R^{3c} represents a hydrogen atom, and R¹ represents any substituents indicated in [Table L1] to [Table L3](hereinafter, referred to as Compound Class SX₁₉₅).

A compound (L-2) wherein T represents a group represented by T14, R^{3b} represents a bromine atom, R^{3c} represents a hydrogen atom, and R¹ represents any substituents indicated in [Table L1] to [Table L3](hereinafter, referred to as Compound Class SX₁₉₆).

A compound (L-2) wherein T represents a group represented by T14, R^{3b} represents an iodine atom, R^{3c} represents a hydrogen atom, and R¹ represents any substituents indicated in [Table L1] to [Table L3](hereinafter, referred to as Compound Class SX₁₉₇).

A compound (L-2) wherein T represents a group represented by T14, R^{3b} represents a hydrogen atom, R^{3c} represents CF₃, and R¹ represents any substituents indicated in [Table L1] to [Table L3](hereinafter, referred to as Compound Class SX₁₉₈).

A compound (L-2) wherein T represents a group represented by T14, R^{3b} represents a hydrogen atom, R^{3c} represents a chlorine atom, and R¹ represents any substituents indicated in [Table L1] to [Table L3](hereinafter, referred to as Compound Class SX₁₉₉).

A compound (L-2) wherein T represents a group represented by T14, R^{3b} represents a hydrogen atom, R^{3c} represents a bromine atom and R¹ represents any substituents indicated in [Table L1] to [Table L3](hereinafter, referred to as Compound Class SX₂₀₀).

A compound (L-2) wherein T represents a group represented by T14, R^{3b} represents a hydrogen atom, R^{3c} represents an iodine atom and R¹ represents any substituents indicated in [Table L1] to [Table L3](hereinafter, referred to as Compound Class SX₂₀₁).

A compound (L-2) wherein T represents a group represented by T15, R^{3b} represents a hydrogen atom, R^{3c} represents a hydrogen atom and R¹ represents any substituents indicated in [Table L1] to [Table L3](hereinafter, referred to as Compound Class SX₂₀₂).

A compound (L-2) wherein T represents a group represented by T15, R^{3b} represents CF₃, R^{3c} represents a hydrogen atom, and R¹ represents any substituents indicated in [Table L1] to [Table L3](hereinafter, referred to as Compound Class SX₂₀₃).

A compound (L-2) wherein T represents a group represented by T15, R^{3b} represents a chlorine atom, R^{3c} represents a hydrogen atom, and R¹ represents any substituents indicated in [Table L1] to [Table L3](hereinafter, referred to as Compound Class SX₂₀₄).

A compound (L-2) wherein T represents a group represented by T15, R^{3b} represents a bromine atom, R^{3c} represents a hydrogen atom, and R¹ represents any substituents indicated in [Table L1] to [Table L3](hereinafter, referred to as Compound Class SX₂₀₅).

A compound (L-2) wherein T represents a group represented by T15, R^{3b} represents an iodine atom, R^{3c} represents a hydrogen atom, and R¹ represents any substituents indicated in [Table L1] to [Table L3](hereinafter, referred to as Compound Class SX₂₀₆).

A compound (L-2) wherein T represents a group represented by T15, R^{3b} represents a hydrogen atom, R^{3c} represents CF₃, and R¹ represents any substituents indicated in [Table L1] to [Table L3](hereinafter, referred to as Compound Class SX₂₀₇).

A compound (L-2) wherein T represents a group represented by T15, R^{3b} represents a hydrogen atom, R^{3c} represents a chlorine atom, and R¹ represents any substituents indicated in [Table L1] to [Table L3](hereinafter, referred to as Compound Class SX₂₀₈).

A compound (L-2) wherein T represents a group represented by T15, R^{3b} represents a hydrogen atom, R^{3c} represents a bromine atom and R¹ represents any substituents indicated in [Table L1] to [Table L3](hereinafter, referred to as Compound Class SX₂₀₉).

A compound (L-2) wherein T represents a group represented by T15, R^{3b} represents a hydrogen atom, R^{3c} represents an iodine atom and R¹ represents any substituents indicated in [Table L1] to [Table L3](hereinafter, referred to as Compound Class SX₂₁₀).

A compound (L-2) wherein T represents a group represented by T16, R^{3b} represents a hydrogen atom, R^{3c} represents a hydrogen atom and R¹ represents any substituents indicated in [Table L1] to [Table L3](hereinafter, referred to as Compound Class SX₂₁₁).

A compound (L-2) wherein T represents a group represented by T16, R^{3b} represents CF₃, R^{3c} represents a hydrogen atom, and R¹ represents any substituents indicated in [Table L1] to [Table L3](hereinafter, referred to as Compound Class SX₂₁₂).

A compound (L-2) wherein T represents a group represented by T16, R^{3b} represents a chlorine atom, R^{3c} represents a hydrogen atom, and R¹ represents any substituents indicated in [Table L1] to [Table L3](hereinafter, referred to as Compound Class SX₂₁₃).

A compound (L-2) wherein T represents a group represented by T16, R^{3b} represents a bromine atom, R^{3c} represents a hydrogen atom, and R¹ represents any substituents indicated in [Table L1] to [Table L3](hereinafter, referred to as Compound Class SX₂₁₄).

A compound (L-2) wherein T represents a group represented by T16, R^{3b} represents an iodine atom, R^{3c} represents a hydrogen atom, and R¹ represents any substituents indicated in [Table L1] to [Table L3](hereinafter, referred to as Compound Class SX₂₁₅).

A compound (L-2) wherein T represents a group represented by T16, R^{3b} represents a hydrogen atom, R^{3c} represents CF₃, and R¹ represents any substituents indicated in [Table L1] to [Table L3](hereinafter, referred to as Compound Class SX₂₁₆).

A compound (L-2) wherein T represents a group represented by T16, R^{3b} represents a hydrogen atom, R^{3c} represents a chlorine atom, and R¹ represents any substituents indicated in [Table L1] to [Table L3](hereinafter, referred to as Compound Class SX₂₁₇).

A compound (L-2) wherein T represents a group represented by T16, R^{3b} represents a hydrogen atom, R^{3c} represents a bromine atom and R¹ represents any substituents indicated in [Table L1] to [Table L3](hereinafter, referred to as Compound Class SX₂₁₈).

A compound (L-2) wherein T represents a group represented by T16, R^{3b} represents a hydrogen atom, R^{3c} represents an iodine atom and R¹ represents any substituents indicated in [Table L1] to [Table L3](hereinafter, referred to as Compound Class SX₂₁₉).

A compound (L-2) wherein T represents a group represented by T17, R^{3b} represents a hydrogen atom, R^{3c} represents a hydrogen atom and R¹ represents any substituents indicated in [Table L1] to [Table L3](hereinafter, referred to as Compound Class SX₂₂₀).

A compound (L-2) wherein T represents a group represented by T17, R^{3b} represents CF₃, R^{3c} represents a hydrogen atom, and R¹ represents any substituents indicated in [Table L1] to [Table L3](hereinafter, referred to as Compound Class SX₂₂₁).

A compound (L-2) wherein T represents a group represented by T17, R^{3b} represents a chlorine atom, R^{3c} represents a hydrogen atom, and R¹ represents any substituents indicated in [Table L1] to [Table L3](hereinafter, referred to as Compound Class SX₂₂₂).

A compound (L-2) wherein T represents a group represented by T17, R^{3b} represents a bromine atom, R^{3c} represents a hydrogen atom, and R¹ represents any substituents indicated in [Table L1] to [Table L3](hereinafter, referred to as Compound Class SX₂₂₃).

A compound (L-2) wherein T represents a group represented by T17, R^{3b} represents an iodine atom, R^{3c} represents a hydrogen atom, and R¹ represents any substituents indicated in [Table L1] to [Table L3](hereinafter, referred to as Compound Class SX₂₂₄).

A compound (L-2) wherein T represents a group represented by T17, R^{3b} represents a hydrogen atom, R^{3c} represents CF₃, and R¹ represents any substituents indicated in [Table L1] to [Table L3](hereinafter, referred to as Compound Class SX₂₂₅).

A compound (L-2) wherein T represents a group represented by T17, R^{3b} represents a hydrogen atom, R^{3c} represents a chlorine atom, and R¹ represents any substituents indicated in [Table L1] to [Table L3](hereinafter, referred to as Compound Class SX₂₂₆).

A compound (L-2) wherein T represents a group represented by T17, R^{3b} represents a hydrogen atom, R^{3c} represents a bromine atom and R¹ represents any substituents indicated in [Table L1] to [Table L3](hereinafter, referred to as Compound Class SX₂₂₇).

A compound (L-2) wherein T represents a group represented by T17, R^{3b} represents a hydrogen atom, R^{3c} represents an iodine atom and R¹ represents any substituents indicated in [Table L1] to [Table L3](hereinafter, referred to as Compound Class SX₂₂₈).

A compound (L-2) wherein T represents a group represented by T18, R^{3b} represents a hydrogen atom, R^{3c} represents a hydrogen atom and R¹ represents any substituents indicated in [Table L1] to [Table L3](hereinafter, referred to as Compound Class SX₂₂₉).

A compound (L-2) wherein T represents a group represented by T18, R^{3b} represents CF₃, R^{3c} represents a hydrogen atom, and R¹ represents any substituents indicated in [Table L1] to [Table L3](hereinafter, referred to as Compound Class SX₂₃₀).

A compound (L-2) wherein T represents a group represented by T18, R^{3b} represents a chlorine atom, R^{3c} represents a hydrogen atom, and R¹ represents any substituents indicated in [Table L1] to [Table L3](hereinafter, referred to as Compound Class SX₂₃₁).

A compound (L-2) wherein T represents a group represented by T18, R^{3b} represents a bromine atom, R^{3c} represents a hydrogen atom, and R¹ represents any substituents indicated in [Table L1] to [Table L3](hereinafter, referred to as Compound Class SX₂₃₂).

A compound (L-2) wherein T represents a group represented by T18, R^{3b} represents an iodine atom, R^{3c} represents a hydrogen atom, and R¹ represents any substituents indicated in [Table L1] to [Table L3](hereinafter, referred to as Compound Class SX₂₃₃).

A compound (L-2) wherein T represents a group represented by T18, R^{3b} represents a hydrogen atom, R^{3c} represents CF₃, and R¹ represents any substituents indicated in [Table L1] to [Table L3](hereinafter, referred to as Compound Class SX₂₃₄).

A compound (L-2) wherein T represents a group represented by T18, R^{3b} represents a hydrogen atom, R^{3c} represents a chlorine atom, and R¹ represents any substituents indicated in [Table L1] to [Table L3](hereinafter, referred to as Compound Class SX₂₃₅).

A compound (L-2) wherein T represents a group represented by T18, R^{3b} represents a hydrogen atom, R^{3c} represents a bromine atom and R¹ represents any substituents indicated in [Table L1] to [Table L3](hereinafter, referred to as Compound Class SX₂₃₆).

A compound (L-2) wherein T represents a group represented by T18, R^{3b} represents a hydrogen atom, R^{3c} represents an iodine atom and R¹ represents any substituents indicated in [Table L1] to [Table L3](hereinafter, referred to as Compound Class SX₂₃₇).

A compound (L-2) wherein T represents a group represented by T19, R^{3b} represents a hydrogen atom, R^{3c} represents a hydrogen atom and R¹ represents any substituents indicated in [Table L1] to [Table L3](hereinafter, referred to as Compound Class SX₂₃₈).

A compound (L-2) wherein T represents a group represented by T19, R^{3b} represents CF₃, R^{3c} represents a hydrogen atom, and R¹ represents any substituents indicated in [Table L1] to [Table L3](hereinafter, referred to as Compound Class SX₂₃₉).

A compound (L-2) wherein T represents a group represented by T19, R^{3b} represents a chlorine atom, R^{3c} represents a hydrogen atom, and R¹ represents any substituents indicated in [Table L1] to [Table L3](hereinafter, referred to as Compound Class SX₂₄₀).

A compound (L-2) wherein T represents a group represented by T19, R^{3b} represents a bromine atom, R^{3c} represents a hydrogen atom, and R¹ represents any substituents indicated in [Table L1] to [Table L3](hereinafter, referred to as Compound Class SX₂₄₁).

A compound (L-2) wherein T represents a group represented by T19, R^{3b} represents an iodine atom, R^{3c} represents a hydrogen atom, and R¹ represents any substituents indicated in [Table L1] to [Table L3](hereinafter, referred to as Compound Class SX₂₄₂).

A compound (L-2) wherein T represents a group represented by T19, R^{3b} represents a hydrogen atom, R^{3c} represents CF₃, and R¹ represents any substituents indicated in [Table L1] to [Table L3](hereinafter, referred to as Compound Class SX₂₄₃).

A compound (L-2) wherein T represents a group represented by T19, R^{3b} represents a hydrogen atom, R^{3c} represents a chlorine atom, and R¹ represents any substituents indicated in [Table L1] to [Table L3](hereinafter, referred to as Compound Class SX₂₄₄).

A compound (L-2) wherein T represents a group represented by T19, R^{3b} represents a hydrogen atom, R^{3c} represents a bromine atom and R¹ represents any substituents indicated in [Table L1] to [Table L3](hereinafter, referred to as Compound Class SX₂₄₅).

A compound (L-2) wherein T represents a group represented by T19, R^{3b} represents a hydrogen atom, R^{3c} represents an iodine atom and R¹ represents any substituents indicated in [Table L1] to [Table L3](hereinafter, referred to as Compound Class SX₂₄₆).

A compound (L-2) wherein T represents a group represented by T20, R^{3b} represents a hydrogen atom, R^{3c} represents a hydrogen atom and R¹ represents any substituents indicated in [Table L1] to [Table L3](hereinafter, referred to as Compound Class SX₂₄₇).

A compound (L-2) wherein T represents a group represented by T20, R^{3b} represents CF₃, R^{3c} represents a hydrogen atom, and R¹ represents any substituents indicated in [Table L1] to [Table L3](hereinafter, referred to as Compound Class SX₂₄₈).

A compound (L-2) wherein T represents a group represented by T20, R^{3b} represents a chlorine atom, R^{3c} represents a hydrogen atom, and R¹ represents any substituents indicated in [Table L1] to [Table L3](hereinafter, referred to as Compound Class SX₂₄₉).

A compound (L-2) wherein T represents a group represented by T20, R^{3b} represents a bromine atom, R^{3c} represents a hydrogen atom, and R¹ represents any substituents indicated in [Table L1] to [Table L3](hereinafter, referred to as Compound Class SX₂₅₀).

A compound (L-2) wherein T represents a group represented by T20, R^{3b} represents an iodine atom, R^{3c} represents a hydrogen atom, and R¹ represents any substituents indicated in [Table L1] to [Table L3](hereinafter, referred to as Compound Class SX₂₅₁).

A compound (L-2) wherein T represents a group represented by T20, R^{3b} represents a hydrogen atom, R^{3c} represents CF₃, and R¹ represents any substituents indicated in [Table L1] to [Table L3](hereinafter, referred to as Compound Class SX₂₅₂).

A compound (L-2) wherein T represents a group represented by T20, R^{3b} represents a hydrogen atom, R^{3c} represents a chlorine atom, and R¹ represents any substituents indicated in [Table L1] to [Table L3](hereinafter, referred to as Compound Class SX₂₅₃).

A compound (L-2) wherein T represents a group represented by T20, R^{3b} represents a hydrogen atom, R^{3c} represents a bromine atom and R¹ represents any substituents indicated in [Table L1] to [Table L3](hereinafter, referred to as Compound Class SX₂₅₄).

A compound (L-2) wherein T represents a group represented by T20, R^{3b} represents a hydrogen atom, R^{3c} represents an iodine atom and R¹ represents any substituents indicated in [Table L1] to [Table L3](hereinafter, referred to as Compound Class SX₂₅₅).

A compound (L-2) wherein T represents a group represented by T21, R^{3b} represents a hydrogen atom, R^{3c} represents a hydrogen atom and R¹ represents any substituents indicated in [Table L1] to [Table L3](hereinafter, referred to as Compound Class SX₂₅₆).

A compound (L-2) wherein T represents a group represented by T20, R^{3b} represents CF₃, R^{3c} represents a hydrogen atom, and R¹ represents any substituents indicated in [Table L1] to [Table L3](hereinafter, referred to as Compound Class SX₂₅₇).

A compound (L-2) wherein T represents a group represented by T20, R^{3b} represents a chlorine atom, R^{3c} represents a hydrogen atom, and R¹ represents any substituents indicated in [Table L1] to [Table L3](hereinafter, referred to as Compound Class SX₂₅₈).

A compound (L-2) wherein T represents a group represented by T20, R^{3b} represents a bromine atom, R^{3c} represents a hydrogen atom, and R¹ represents any substituents indicated in [Table L1] to [Table L3](hereinafter, referred to as Compound Class SX₂₅₉).

A compound (L-2) wherein T represents a group represented by T20, R^{3b} represents an iodine atom, R^{3c} represents a hydrogen atom, and R¹ represents any substituents indicated in [Table L1] to [Table L3](hereinafter, referred to as Compound Class SX₂₆₀).

A compound (L-2) wherein T represents a group represented by T20, R^{3b} represents a hydrogen atom, R^{3c} represents CF₃, and R¹ represents any substituents indicated in [Table L1] to [Table L3](hereinafter, referred to as Compound Class SX₂₆₁).

A compound (L-2) wherein T represents a group represented by T20, R^{3b} represents a hydrogen atom, R^{3c} represents a chlorine atom, and R¹ represents any substituents indicated in [Table L1] to [Table L3](hereinafter, referred to as Compound Class SX₂₆₂).

A compound (L-2) wherein T represents a group represented by T20, R^{3b} represents a hydrogen atom, R^{3c} represents a bromine atom and R¹ represents any substituents indicated in [Table L1] to [Table L3](hereinafter, referred to as Compound Class SX₂₆₃).

A compound (L-2) wherein T represents a group represented by T20, R^{3b} represents a hydrogen atom, R^{3c} represents an iodine atom and R¹ represents any substituents indicated in [Table L1] to [Table L3](hereinafter, referred to as Compound Class SX₂₆₄).

A compound (L-2) wherein T represents a group represented by T22, R^{3b} represents a hydrogen atom, R^{3c} represents a hydrogen atom and R¹ represents any substituents indicated in [Table L1] to [Table L3](hereinafter, referred to as Compound Class SX₂₆₅).

A compound (L-2) wherein T represents a group represented by T22, R^{3b} represents CF₃, R^{3c} represents a hydrogen atom, and R¹ represents any substituents indicated in [Table L1] to [Table L3](hereinafter, referred to as Compound Class SX₂₆₆).

A compound (L-2) wherein T represents a group represented by T22, R^{3b} represents a chlorine atom, R^{3c} represents a hydrogen atom, and R¹ represents any substituents indicated in [Table L1] to [Table L3](hereinafter, referred to as Compound Class SX₂₆₇).

A compound (L-2) wherein T represents a group represented by T22, R^{3b} represents a bromine atom, R^{3c} represents a hydrogen atom, and R¹ represents any substituents indicated in [Table L1] to [Table L3](hereinafter, referred to as Compound Class SX₂₆₈).

A compound (L-2) wherein T represents a group represented by T22, R^{3b} represents an iodine atom, R^{3c} represents a hydrogen atom, and R¹ represents any substituents indicated in [Table L1] to [Table L3](hereinafter, referred to as Compound Class SX₂₆₉).

A compound (L-2) wherein T represents a group represented by T22, R^{3b} represents a hydrogen atom, R^{3c} represents CF₃, and R¹ represents any substituents indicated in [Table L1] to [Table L3](hereinafter, referred to as Compound Class SX₂₇₀).

A compound (L-2) wherein T represents a group represented by T22, R^{3b} represents a hydrogen atom, R^{3c} represents a chlorine atom, and R¹ represents any substituents indicated in [Table L1] to [Table L3](hereinafter, referred to as Compound Class SX₂₇₁).

A compound (L-2) wherein T represents a group represented by T22, R^{3b} represents a hydrogen atom, R^{3c} represents a bromine atom and R¹ represents any substituents indicated in [Table L1] to [Table L3](hereinafter, referred to as Compound Class SX₂₇₂).

A compound (L-2) wherein T represents a group represented by T22, R^{3b} represents a hydrogen atom, R^{3c} represents an iodine atom and R¹ represents any substituents indicated in [Table L1] to [Table L3](hereinafter, referred to as Compound Class SX₂₇₃).

A compound (L-2) wherein T represents a group represented by T23, R^{3b} represents a hydrogen atom, R^{3c} represents a hydrogen atom and R¹ represents any substituents indicated in [Table L1] to [Table L3](hereinafter, referred to as Compound Class SX₂₇₄).

A compound (L-2) wherein T represents a group represented by T23, R^{3b} represents CF₃, R^{3c} represents a hydrogen atom, and R¹ represents any substituents indicated in [Table L1] to [Table L3](hereinafter, referred to as Compound Class SX₂₇₅).

A compound (L-2) wherein T represents a group represented by T23, R^{3b} represents a chlorine atom, R^{3c} represents a hydrogen atom, and R¹ represents any substituents indicated in [Table L1] to [Table L3](hereinafter, referred to as Compound Class SX₂₇₆).

A compound (L-2) wherein T represents a group represented by T23, R^{3b} represents a bromine atom, R^{3c} represents a hydrogen atom, and R¹ represents any substituents indicated in [Table L1] to [Table L3](hereinafter, referred to as Compound Class SX₂₇₇).

A compound (L-2) wherein T represents a group represented by T23, R^{3b} represents an iodine atom, R^{3c} represents a hydrogen atom, and R¹ represents any substituents indicated in [Table L1] to [Table L3](hereinafter, referred to as Compound Class SX₂₇₈).

A compound (L-2) wherein T represents a group represented by T23, R^{3b} represents a hydrogen atom, R^{3c} represents CF₃, and R¹ represents any substituents indicated in [Table L1] to [Table L3](hereinafter, referred to as Compound Class SX₂₇₉).

A compound (L-2) wherein T represents a group represented by T23, R^{3b} represents a hydrogen atom, R^{3c} represents a chlorine atom, and R¹ represents any substituents indicated in [Table L1] to [Table L3](hereinafter, referred to as Compound Class SX₂₈₀).

A compound (L-2) wherein T represents a group represented by T23, R^{3b} represents a hydrogen atom, R^{3c} represents a bromine atom and R¹ represents any substituents indicated in [Table L1] to [Table L3](hereinafter, referred to as Compound Class SX₂₈₁).

A compound (L-2) wherein T represents a group represented by T23, R^{3b} represents a hydrogen atom, R^{3c} represents an iodine atom and R¹ represents any substituents indicated in [Table L1] to [Table L3](hereinafter, referred to as Compound Class SX₂₈₂).

A compound (L-2) wherein T represents a group represented by T24, R^{3b} represents a hydrogen atom, R^{3c} represents a hydrogen atom and R¹ represents any substituents indicated in [Table L1] to [Table L3](hereinafter, referred to as Compound Class SX₂₈₃).

A compound (L-2) wherein T represents a group represented by T24, R^{3b} represents CF₃, R^{3c} represents a hydrogen atom, and R¹ represents any substituents indicated in [Table L1] to [Table L3](hereinafter, referred to as Compound Class SX₂₈₄).

A compound (L-2) wherein T represents a group represented by T24, R^{3b} represents a chlorine atom, R^{3c} represents a hydrogen atom, and R¹ represents any substituents indicated in [Table L1] to [Table L3](hereinafter, referred to as Compound Class SX₂₈₅).

A compound (L-2) wherein T represents a group represented by T24, R^{3b} represents a bromine atom, R^{3c} represents a hydrogen atom, and R¹ represents any substituents indicated in [Table L1] to [Table L3](hereinafter, referred to as Compound Class SX₂₈₆).

A compound (L-2) wherein T represents a group represented by T24, R^{3b} represents an iodine atom, R^{3c} represents a hydrogen atom, and R¹ represents any substituents indicated in [Table L1] to [Table L3](hereinafter, referred to as Compound Class SX₂₈₇).

A compound (L-2) wherein T represents a group represented by T24, R^{3b} represents a hydrogen atom, R^{3c} represents CF₃, and R¹ represents any substituents indicated in [Table L1] to [Table L3](hereinafter, referred to as Compound Class SX₂₈₈).

A compound (L-2) wherein T represents a group represented by T24, R^{3b} represents a hydrogen atom, R^{3c} represents a chlorine atom, and R¹ represents any substituents indicated in [Table L1] to [Table L3](hereinafter, referred to as Compound Class SX₂₈₉).

A compound (L-2) wherein T represents a group represented by T24, R^{3b} represents a hydrogen atom, R^{3c} represents a bromine atom and R¹ represents any substituents indicated in [Table L1] to [Table L3](hereinafter, referred to as Compound Class SX₂₉₀).

A compound (L-2) wherein T represents a group represented by T24, R^{3b} represents a hydrogen atom, R^{3c} represents an iodine atom and R¹ represents any substituents indicated in [Table L1] to [Table L3](hereinafter, referred to as Compound Class SX₂₉₁).

A compound (L-2) wherein T represents a group represented by T25, R^{3b} represents a hydrogen atom, R^{3c} represents a hydrogen atom and R¹ represents any substituents indicated in [Table L1] to [Table L3](hereinafter, referred to as Compound Class SX₂₉₂).

A compound (L-2) wherein T represents a group represented by T25, R^{3b} represents CF₃, R^{3c} represents a hydrogen atom, and R¹ represents any substituents indicated in [Table L1] to [Table L3](hereinafter, referred to as Compound Class SX₂₉₃).

A compound (L-2) wherein T represents a group represented by T25, R^{3b} represents a chlorine atom, R^{3c} represents a hydrogen atom, and R¹ represents any substituents indicated in [Table L1] to [Table L3](hereinafter, referred to as Compound Class SX₂₉₄).

A compound (L-2) wherein T represents a group represented by T25, R^{3b} represents a bromine atom, R^{3c} represents a hydrogen atom, and R¹ represents any substituents indicated in [Table L1] to [Table L3](hereinafter, referred to as Compound Class SX₂₉₅).

A compound (L-2) wherein T represents a group represented by T25, R^{3b} represents an iodine atom, R^{3c} represents a hydrogen atom, and R¹ represents any substituents indicated in [Table L1] to [Table L3](hereinafter, referred to as Compound Class SX₂₉₆).

A compound (L-2) wherein T represents a group represented by T25, R^{3b} represents a hydrogen atom, R^{3c} represents CF₃, and R¹ represents any substituents indicated in [Table L1] to [Table L3](hereinafter, referred to as Compound Class SX₂₉₇).

A compound (L-2) wherein T represents a group represented by T25, R^{3b} represents a hydrogen atom, R^{3c} represents a chlorine atom, and R¹ represents any substituents indicated in [Table L1] to [Table L3](hereinafter, referred to as Compound Class SX₂₉₈).

A compound (L-2) wherein T represents a group represented by T25, R^{3b} represents a hydrogen atom, R^{3c} represents a bromine atom and R¹ represents any substituents indicated in [Table L1] to [Table L3](hereinafter, referred to as Compound Class SX₂₉₉).

A compound (L-2) wherein T represents a group represented by T25, R^{3b} represents a hydrogen atom, R^{3c} represents an iodine atom and R¹ represents any substituents indicated in [Table L1] to [Table L3](hereinafter, referred to as Compound Class SX₃₀₀).

A compound (L-2) wherein T represents a group represented by T26, R^{3b} represents a hydrogen atom, R^{3c} represents a hydrogen atom and R¹ represents any substituents indicated in [Table L1] to [Table L3](hereinafter, referred to as Compound Class SX₃₀₁).

A compound (L-2) wherein T represents a group represented by T26, R^{3b} represents CF₃, R^{3c} represents a hydrogen atom, and R¹ represents any substituents indicated in [Table L1] to [Table L3](hereinafter, referred to as Compound Class SX₃₀₂).

A compound (L-2) wherein T represents a group represented by T26, R^{3b} represents a chlorine atom, R^{3c} represents a hydrogen atom, and R¹ represents any substituents indicated in [Table L1] to [Table L3](hereinafter, referred to as Compound Class SX₃₀₃).

A compound (L-2) wherein T represents a group represented by T26, R^{3b} represents a bromine atom, R^{3c} represents a hydrogen atom, and R¹ represents any substituents indicated in [Table L1] to [Table L3](hereinafter, referred to as Compound Class SX₃₀₄).

A compound (L-2) wherein T represents a group represented by T26, R^{3b} represents an iodine atom, R^{3c} represents a hydrogen atom, and R¹ represents any substituents indicated in [Table L1] to [Table L3](hereinafter, referred to as Compound Class SX₃₀₅).

A compound (L-2) wherein T represents a group represented by T26, R^{3b} represents a hydrogen atom, R^{3c} represents CF₃, and R¹ represents any substituents indicated in [Table L1] to [Table L3](hereinafter, referred to as Compound Class SX₃₀₆).

A compound (L-2) wherein T represents a group represented by T26, R^{3b} represents a hydrogen atom, R^{3c} represents a chlorine atom, and R¹ represents any substituents indicated in [Table L1] to [Table L3](hereinafter, referred to as Compound Class SX₃₀₇).

A compound (L-2) wherein T represents a group represented by T26, R^{3b} represents a hydrogen atom, R^{3c} represents a bromine atom and R¹ represents any substituents indicated in [Table L1] to [Table L3](hereinafter, referred to as Compound Class SX₃₀₈).

A compound (L-2) wherein T represents a group represented by T26, R^{3b} represents a hydrogen atom, R^{3c} represents an iodine atom and R¹ represents any substituents indicated in [Table L1] to [Table L3](hereinafter, referred to as Compound Class SX₃₀₉).

A compound (L-2) wherein T represents a group represented by T27, R^{3b} represents a hydrogen atom, R^{3c} represents a hydrogen atom and R¹ represents any substituents indicated in [Table L1] to [Table L3](hereinafter, referred to as Compound Class SX₃₁₀).

A compound (L-2) wherein T represents a group represented by T27, R^{3b} represents CF₃, R^{3c} represents a hydrogen atom, and R¹ represents any substituents indicated in [Table L1] to [Table L3](hereinafter, referred to as Compound Class SX₃₁₁).

A compound (L-2) wherein T represents a group represented by T27, R^{3b} represents a chlorine atom, R^{3c} represents a hydrogen atom, and R¹ represents any substituents indicated in [Table L1] to [Table L3](hereinafter, referred to as Compound Class SX₃₁₂).

A compound (L-2) wherein T represents a group represented by T27, R^{3b} represents a bromine atom, R^{3c} represents a hydrogen atom, and R¹ represents any substituents indicated in [Table L1] to [Table L3](hereinafter, referred to as Compound Class SX₃₁₃).

A compound (L-2) wherein T represents a group represented by T27, R^{3b} represents an iodine atom, R^{3c} represents a hydrogen atom, and R¹ represents any substituents indicated in [Table L1] to [Table L3](hereinafter, referred to as Compound Class SX₃₁₄).

A compound (L-2) wherein T represents a group represented by T27, R^{3b} represents a hydrogen atom, R^{3c} represents CF₃, and R¹ represents any substituents indicated in [Table L1] to [Table L3](hereinafter, referred to as Compound Class SX₃₁₅).

A compound (L-2) wherein T represents a group represented by T27, R^{3b} represents a hydrogen atom, R^{3c} represents a chlorine atom, and R¹ represents any substituents indicated in [Table L1] to [Table L3](hereinafter, referred to as Compound Class SX₃₁₆).

A compound (L-2) wherein T represents a group represented by T27, R^{3b} represents a hydrogen atom, R^{3c} represents a bromine atom and R¹ represents any substituents indicated in [Table L1] to [Table L3](hereinafter, referred to as Compound Class SX₃₁₇).

A compound (L-2) wherein T represents a group represented by T27, R^{3b} represents a hydrogen atom, R^{3c} represents an iodine atom and R¹ represents any substituents indicated in [Table L1] to [Table L3](hereinafter, referred to as Compound Class SX₃₁₈).

A compound (L-2) wherein T represents a group represented by T28, R^{3b} represents a hydrogen atom, R^{3c} represents a hydrogen atom and R¹ represents any substituents indicated in [Table L1] to [Table L3](hereinafter, referred to as Compound Class SX₃₁₉).

A compound (L-2) wherein T represents a group represented by T28, R^{3b} represents CF₃, R^{3c} represents a hydrogen atom, and R¹ represents any substituents indicated in [Table L1] to [Table L3](hereinafter, referred to as Compound Class SX₃₂₀).

A compound (L-2) wherein T represents a group represented by T28, R^{3b} represents a chlorine atom, R^{3c} represents a hydrogen atom, and R¹ represents any substituents indicated in [Table L1] to [Table L3](hereinafter, referred to as Compound Class SX₃₂₁).

A compound (L-2) wherein T represents a group represented by T28, R^{3b} represents a bromine atom, R^{3c} represents a hydrogen atom, and R¹ represents any substituents indicated in [Table L1] to [Table L3](hereinafter, referred to as Compound Class SX₃₂₂).

A compound (L-2) wherein T represents a group represented by T28, R^{3b} represents an iodine atom, R^{3c} represents a hydrogen atom, and R¹ represents any substituents indicated in [Table L1] to [Table L3](hereinafter, referred to as Compound Class SX₃₂₃).

A compound (L-2) wherein T represents a group represented by T28, R^{3b} represents a hydrogen atom, R^{3c} represents CF₃, and R¹ represents any substituents indicated in [Table L1] to [Table L3](hereinafter, referred to as Compound Class SX₃₂₄).

A compound (L-2) wherein T represents a group represented by T28, R^{3b} represents a hydrogen atom, R^{3c} represents a chlorine atom, and R¹ represents any substituents indicated in [Table L1] to [Table L3](hereinafter, referred to as Compound Class SX₃₂₅).

A compound (L-2) wherein T represents a group represented by T28, R^{3b} represents a hydrogen atom, R^{3c} represents a bromine atom and R¹ represents any substituents indicated in [Table L1] to [Table L3](hereinafter, referred to as Compound Class SX₃₂₆).

A compound (L-2) wherein T represents a group represented by T28, R^{3b} represents a hydrogen atom, R^{3c} represents an iodine atom and R¹ represents any substituents indicated in [Table L1] to [Table L3](hereinafter, referred to as Compound Class SX₃₂₇).

A compound (L-2) wherein T represents a group represented by T29, R^{3b} represents a hydrogen atom, R^{3c} represents a hydrogen atom and R¹ represents any substituents indicated in [Table L1] to [Table L3](hereinafter, referred to as Compound Class SX₃₂₈).

A compound (L-2) wherein T represents a group represented by T29, R^{3b} represents CF₃, R^{3c} represents a hydrogen atom, and R¹ represents any substituents indicated in [Table L1] to [Table L3](hereinafter, referred to as Compound Class SX₃₂₉).

A compound (L-2) wherein T represents a group represented by T29, R^{3b} represents a chlorine atom, R^{3c} represents a hydrogen atom, and R¹ represents any substituents indicated in [Table L1] to [Table L3](hereinafter, referred to as Compound Class SX₃₃₀).

A compound (L-2) wherein T represents a group represented by T29, R^{3b} represents a bromine atom, R^{3c} represents a hydrogen atom, and R¹ represents any substituents indicated in [Table L1] to [Table L3](hereinafter, referred to as Compound Class SX₃₃₁).

A compound (L-2) wherein T represents a group represented by T29, R^{3b} represents an iodine atom, R^{3c} represents a hydrogen atom, and R¹ represents any substituents indicated in [Table L1] to [Table L3](hereinafter, referred to as Compound Class SX₃₃₂).

A compound (L-2) wherein T represents a group represented by T29, R^{3b} represents a hydrogen atom, R^{3c} represents CF₃, and R¹ represents any substituents indicated in [Table L1] to [Table L3](hereinafter, referred to as Compound Class SX₃₃₃).

A compound (L-2) wherein T represents a group represented by T29, R^{3b} represents a hydrogen atom, R^{3c} represents a chlorine atom, and R¹ represents any substituents indicated in [Table L1] to [Table L3](hereinafter, referred to as Compound Class SX₃₃₄).

A compound (L-2) wherein T represents a group represented by T29, R^{3b} represents a hydrogen atom, R^{3c} represents a bromine atom and R¹ represents any substituents indicated in [Table L1] to [Table L3](hereinafter, referred to as Compound Class SX₃₃₅).

A compound (L-2) wherein T represents a group represented by T29, R^{3b} represents a hydrogen atom, R^{3c} represents an iodine atom and R¹ represents any substituents indicated in [Table L1] to [Table L3](hereinafter, referred to as Compound Class SX₃₃₆).

A compound (L-2) wherein T represents a group represented by T30, R^{3b} represents a hydrogen atom, R^{3c} represents a hydrogen atom and R¹ represents any substituents indicated in [Table L1] to [Table L3](hereinafter, referred to as Compound Class SX₃₃₇).

A compound (L-2) wherein T represents a group represented by T30, R^{3b} represents CF₃, R^{3c} represents a hydrogen atom, and R¹ represents any substituents indicated in [Table L1] to [Table L3](hereinafter, referred to as Compound Class SX₃₃₈).

A compound (L-2) wherein T represents a group represented by T30, R^{3b} represents a chlorine atom, R^{3c} represents a hydrogen atom, and R¹ represents any substituents indicated in [Table L1] to [Table L3](hereinafter, referred to as Compound Class SX₃₃₉).

A compound (L-2) wherein T represents a group represented by T30, R^{3b} represents a bromine atom, R^{3c} represents a hydrogen atom, and R¹ represents any substituents indicated in [Table L1] to [Table L3](hereinafter, referred to as Compound Class SX₃₄₀).

A compound (L-2) wherein T represents a group represented by T30, R^{3b} represents an iodine atom, R^{3c} represents a hydrogen atom, and R¹ represents any substituents indicated in [Table L1] to [Table L3](hereinafter, referred to as Compound Class SX₃₄₁).

A compound (L-2) wherein T represents a group represented by T30, R^{3b} represents a hydrogen atom, R^{3c} represents CF₃, and R¹ represents any substituents indicated in [Table L1] to [Table L3](hereinafter, referred to as Compound Class SX₃₄₂).

A compound (L-2) wherein T represents a group represented by T30, R^{3b} represents a hydrogen atom, R^{3c} represents a chlorine atom, and R¹ represents any substituents indicated in [Table L1] to [Table L3](hereinafter, referred to as Compound Class SX₃₄₃).

A compound (L-2) wherein T represents a group represented by T30, R^{3b} represents a hydrogen atom, R^{3c} represents a bromine atom and R¹ represents any substituents indicated in [Table L1] to [Table L3](hereinafter, referred to as Compound Class SX₃₄₄).

A compound (L-2) wherein T represents a group represented by T30, R^{3b} represents a hydrogen atom, R^{3c} represents an iodine atom and R¹ represents any substituents indicated in [Table L1] to [Table L3](hereinafter, referred to as Compound Class SX₃₄₅).

A compound (L-2) wherein T represents a group represented by T31, R^{3b} represents a hydrogen atom, R^{3c} represents a hydrogen atom and R¹ represents any substituents indicated in [Table L1] to [Table L3](hereinafter, referred to as Compound Class SX₃₄₆).

A compound (L-2) wherein T represents a group represented by T31, R^{3b} represents CF₃, R^{3c} represents a hydrogen atom, and R¹ represents any substituents indicated in [Table L1] to [Table L3](hereinafter, referred to as Compound Class SX₃₄₇).

A compound (L-2) wherein T represents a group represented by T31, R^{3b} represents a chlorine atom, R^{3c} represents a hydrogen atom, and R¹ represents any substituents indicated in [Table L1] to [Table L3](hereinafter, referred to as Compound Class SX₃₄₈).

A compound (L-2) wherein T represents a group represented by T31, R^{3b} represents a bromine atom, R^{3c} represents a hydrogen atom, and R¹ represents any substituents indicated in [Table L1] to [Table L3](hereinafter, referred to as Compound Class SX₃₄₉).

A compound (L-2) wherein T represents a group represented by T31, R^{3b} represents an iodine atom, R^{3c} represents a hydrogen atom, and R¹ represents any substituents indicated in [Table L1] to [Table L3](hereinafter, referred to as Compound Class SX₃₅₀).

A compound (L-2) wherein T represents a group represented by T31, R^{3b} represents a hydrogen atom, R^{3c} represents CF₃, and R¹ represents any substituents indicated in [Table L1] to [Table L3](hereinafter, referred to as Compound Class SX₃₅₁).

A compound (L-2) wherein T represents a group represented by T31, R^{3b} represents a hydrogen atom, R^{3c} represents a chlorine atom, and R¹ represents any substituents indicated in [Table L1] to [Table L3](hereinafter, referred to as Compound Class SX₃₅₂).

A compound (L-2) wherein T represents a group represented by T31, R^{3b} represents a hydrogen atom, R^{3c} represents a bromine atom and R¹ represents any substituents indicated in [Table L1] to [Table L3](hereinafter, referred to as Compound Class SX₃₅₃).

A compound (L-2) wherein T represents a group represented by T31, R^{3b} represents a hydrogen atom, R^{3c} represents an iodine atom and R¹ represents any substituents indicated in [Table L1] to [Table L3](hereinafter, referred to as Compound Class SX₃₅₄).

A compound represented by formula (L-3):

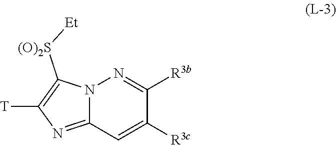

(hereinafter, referred to as compound (L-3)), wherein T represents a group represented by T1, R^{3b} represents a hydrogen atom, R^{3c} represents a hydrogen atom, and R¹ represents any substituents indicated in [Table L1] to [Table L3](hereinafter, referred to as Compound Class SX₃₅₅).

A compound (L-3) wherein T represents a group represented by T1, R^{3b} represents CF₃, R^{3c} represents a hydrogen atom, and R¹ represents any substituents indicated in [Table L1] to [Table L3](hereinafter, referred to as Compound Class SX₃₅₆).

A compound (L-3) wherein T represents a group represented by T1, R^{3b} represents a chlorine atom, R^{3c} represents a hydrogen atom, and R¹ represents any substituents indicated in [Table L1] to [Table L3](hereinafter, referred to as Compound Class SX₃₅₇).

A compound (L-3) wherein T represents a group represented by T1, R^{3b} represents a bromine atom, R^{3c} represents a hydrogen atom, and R¹ represents any substituents indicated in [Table L1] to [Table L3](hereinafter, referred to as Compound Class SX₃₅₈).

A compound (L-3) wherein T represents a group represented by T1, R^{3b} represents an iodine atom, R^{3c} represents a hydrogen atom, and R¹ represents any substituents indicated in [Table L1] to [Table L3](hereinafter, referred to as Compound Class SX₃₅₉).

A compound (L-3) wherein T represents a group represented by T1, R^{3b} represents a hydrogen atom, R^{3c} represents CF₃, and R¹ represents any substituents indicated in [Table L1] to [Table L3](hereinafter, referred to as Compound Class SX₃₆₀).

A compound (L-3) wherein T represents a group represented by T1, R^{3b} represents a hydrogen atom, R^{3c} represents a chlorine atom, and R¹ represents any substituents indicated in [Table L1] to [Table L3](hereinafter, referred to as Compound Class SX₃₆₁).

A compound (L-3) wherein T represents a group represented by T1, R^{3b} represents a hydrogen atom, R^{3c} represents a bromine atom and R¹ represents any substituents indicated in [Table L1] to [Table L3](hereinafter, referred to as Compound Class SX₃₆₂).

A compound (L-3) wherein T represents a group represented by T1, R^{3b} represents a hydrogen atom, R^{3c} represents an iodine atom and R¹ represents any substituents indicated in [Table L1] to [Table L3](hereinafter, referred to as Compound Class SX₃₆₃).

A compound (L-3) wherein T represents a group represented by T2, R^{3b} represents a hydrogen atom, R^{3c} represents a hydrogen atom, and R¹ represents any substituents indicated in [Table L1] to [Table L3](hereinafter, referred to as Compound Class SX₃₆₄).

A compound (L-3) wherein T represents a group represented by T2, R^{3b} represents CF₃, R^{3c} represents a hydrogen atom, and R¹ represents any substituents indicated in [Table L1] to [Table L3](hereinafter, referred to as Compound Class SX₃₆₅).

A compound (L-3) wherein T represents a group represented by T2, R^{3b} represents a chlorine atom, R^{3c} represents a hydrogen atom, and R¹ represents any substituents indicated in [Table L1] to [Table L3](hereinafter, referred to as Compound Class SX₃₆₆).

A compound (L-3) wherein T represents a group represented by T2, R^{3b} represents a bromine atom, R^{3c} represents a hydrogen atom, and R¹ represents any substituents indicated in [Table L1] to [Table L3](hereinafter, referred to as Compound Class SX₃₆₇).

A compound (L-3) wherein T represents a group represented by T2, R^{3b} represents an iodine atom, R^{3c} represents a hydrogen atom, and R¹ represents any substituents indicated in [Table L1] to [Table L3](hereinafter, referred to as Compound Class SX₃₆₈).

A compound (L-3) wherein T represents a group represented by T2, R^{3b} represents a hydrogen atom, R^{3c} represents CF₃, and R¹ represents any substituents indicated in [Table L1] to [Table L3](hereinafter, referred to as Compound Class SX₃₆₉).

A compound (L-3) wherein T represents a group represented by T2, R^{3b} represents a hydrogen atom, R^{3c} represents a chlorine atom, and R¹ represents any substituents indicated in [Table L1] to [Table L3](hereinafter, referred to as Compound Class SX₃₇₀).

A compound (L-3) wherein T represents a group represented by T2, R^{3b} represents a hydrogen atom, R^{3c} represents a bromine atom and R¹ represents any substituents indicated in [Table L1] to [Table L3](hereinafter, referred to as Compound Class SX₃₇₁).

A compound (L-3) wherein T represents a group represented by T2, R^{3b} represents a hydrogen atom, R^{3c} represents an iodine atom and R¹ represents any substituents indicated in [Table L1] to [Table L3](hereinafter, referred to as Compound Class SX₃₇₂).

A compound (L-3) wherein T represents a group represented by T3, R^{3b} represents a hydrogen atom, R^{3c} represents a hydrogen atom, and R¹ represents any substituents indicated in [Table L1] to [Table L3](hereinafter, referred to as Compound Class SX₃₇₃).

A compound (L-3) wherein T represents a group represented by T3, R^{3b} represents CF₃, R^{3c} represents a hydrogen atom, and R¹ represents any substituents indicated in [Table L1] to [Table L3](hereinafter, referred to as Compound Class SX₃₇₄).

A compound (L-3) wherein T represents a group represented by T3, R^{3b} represents a chlorine atom, R^{3c} represents a hydrogen atom, and R¹ represents any substituents indicated in [Table L1] to [Table L3](hereinafter, referred to as Compound Class SX₃₇₅).

A compound (L-3) wherein T represents a group represented by T3, R^{3b} represents a bromine atom, R^{3c} represents a hydrogen atom, and R¹ represents any substituents indicated in [Table L1] to [Table L3](hereinafter, referred to as Compound Class SX₃₇₆).

A compound (L-3) wherein T represents a group represented by T3, R^{3b} represents an iodine atom, R^{3c} represents a hydrogen atom, and R¹ represents any substituents indicated in [Table L1] to [Table L3](hereinafter, referred to as Compound Class SX₃₇₇).

A compound (L-3) wherein T represents a group represented by T3, R^{3b} represents a hydrogen atom, R^{3c} represents CF₃, and R¹ represents any substituents indicated in [Table L1] to [Table L3](hereinafter, referred to as Compound Class SX₃₇₈).

A compound (L-3) wherein T represents a group represented by T3, R^{3b} represents a hydrogen atom, R^{3c} represents a chlorine atom, and R¹ represents any substituents indicated in [Table L1] to [Table L3](hereinafter, referred to as Compound Class SX₃₇₉).

A compound (L-3) wherein T represents a group represented by T3, R^{3b} represents a hydrogen atom, R^{3c} represents a bromine atom and R¹ represents any substituents indicated in [Table L1] to [Table L3](hereinafter, referred to as Compound Class SX₃₈₀).

A compound (L-3) wherein T represents a group represented by T3, R^{3b} represents a hydrogen atom, R^{3c} represents an iodine atom and R¹ represents any substituents indicated in [Table L1] to [Table L3](hereinafter, referred to as Compound Class SX₃₈₁).

A compound (L-3) wherein T represents a group represented by T4, R^{3b} represents a hydrogen atom, R^{3c} represents a hydrogen atom, and R¹ represents any substituents indicated in [Table L1] to [Table L3](hereinafter, referred to as Compound Class SX₃₈₂).

A compound (L-3) wherein T represents a group represented by T4, R^{3b} represents CF₃, R^{3c} represents a hydrogen atom, and R¹ represents any substituents indicated in [Table L1] to [Table L3](hereinafter, referred to as Compound Class SX₃₈₃).

A compound (L-3) wherein T represents a group represented by T4, R^{3b} represents a chlorine atom, R^{3c} represents a hydrogen atom, and R¹ represents any substituents indicated in [Table L1] to [Table L3](hereinafter, referred to as Compound Class SX₃₈₄).

A compound (L-3) wherein T represents a group represented by T4, R^{3b} represents a bromine atom, R^{3c} represents a hydrogen atom, and R¹ represents any substituents indicated in [Table L1] to [Table L3](hereinafter, referred to as Compound Class SX₃₈₅).

A compound (L-3) wherein T represents a group represented by T4, R^{3b} represents an iodine atom, R^{3c} represents a hydrogen atom, and R¹ represents any substituents indicated in [Table L1] to [Table L3](hereinafter, referred to as Compound Class SX₃₈₆).

A compound (L-3) wherein T represents a group represented by T4, R^{3b} represents a hydrogen atom, R^{3c} represents CF₃, and R¹ represents any substituents indicated in [Table L1] to [Table L3](hereinafter, referred to as Compound Class SX₃₈₇).

A compound (L-3) wherein T represents a group represented by T4, R^{3b} represents a hydrogen atom, R^{3c} represents a chlorine atom, and R¹ represents any substituents indicated in [Table L1] to [Table L3](hereinafter, referred to as Compound Class SX₃₈₈).

A compound (L-3) wherein T represents a group represented by T4, R^{3b} represents a hydrogen atom, R^{3c} represents a bromine atom and R¹ represents any substituents indicated in [Table L1] to [Table L3](hereinafter, referred to as Compound Class SX₃₈₉).

A compound (L-3) wherein T represents a group represented by T4, R^{3b} represents a hydrogen atom, R^{3c} represents an iodine atom and R¹ represents any substituents indicated in [Table L1] to [Table L3](hereinafter, referred to as Compound Class SX₃₉₀).

A compound (L-3) wherein T represents a group represented by T5, R^{3b} represents a hydrogen atom, R^{3c} represents a hydrogen atom, and R¹ represents any substituents indicated in [Table L1] to [Table L3](hereinafter, referred to as Compound Class SX₃₉₁).

A compound (L-3) wherein T represents a group represented by T5, R^{3b} represents CF₃, R^{3c} represents a hydrogen atom, and R¹ represents any substituents indicated in [Table L1] to [Table L3](hereinafter, referred to as Compound Class SX₃₉₂).

A compound (L-3) wherein T represents a group represented by T5, R^{3b} represents a chlorine atom, R^{3c} represents a hydrogen atom, and R¹ represents any substituents indicated in [Table L1] to [Table L3](hereinafter, referred to as Compound Class SX₃₉₃).

A compound (L-3) wherein T represents a group represented by T5, R^{3b} represents a bromine atom, R^{3c} represents a hydrogen atom, and R¹ represents any substituents indicated in [Table L1] to [Table L3](hereinafter, referred to as Compound Class SX₃₉₄).

A compound (L-3) wherein T represents a group represented by T5, R^{3b} represents an iodine atom, R^{3c} represents a hydrogen atom, and R¹ represents any substituents indicated in [Table L1] to [Table L3](hereinafter, referred to as Compound Class SX₃₉₅).

A compound (L-3) wherein T represents a group represented by T5, R^{3b} represents a hydrogen atom, R^{3c} represents CF₃, and R¹ represents any substituents indicated in [Table L1] to [Table L3](hereinafter, referred to as Compound Class SX₃₉₆).

A compound (L-3) wherein T represents a group represented by T5, R^{3b} represents a hydrogen atom, R^{3c} represents a chlorine atom, and R¹ represents any substituents indicated in [Table L1] to [Table L3](hereinafter, referred to as Compound Class SX₃₉₇).

A compound (L-3) wherein T represents a group represented by T5, R^{3b} represents a hydrogen atom, R^{3c} represents a bromine atom and R¹ represents any substituents indicated in [Table L1] to [Table L3](hereinafter, referred to as Compound Class SX₃₉₈).

A compound (L-3) wherein T represents a group represented by T5, R^{3b} represents a hydrogen atom, R^{3c} represents an iodine atom and R¹ represents any substituents indicated in [Table L1] to [Table L3](hereinafter, referred to as Compound Class SX₃₉₉).

A compound (L-3) wherein T represents a group represented by T6, R^{3b} represents a hydrogen atom, R^{3c} represents a hydrogen atom, and R¹ represents any substituents indicated in [Table L1] to [Table L3](hereinafter, referred to as Compound Class SX₄₀₀).

A compound (L-3) wherein T represents a group represented by T6, R^{3b} represents CF₃, R^{3c} represents a hydrogen atom, and R¹ represents any substituents indicated in [Table L1] to [Table L3](hereinafter, referred to as Compound Class SX₄₀₁).

A compound (L-3) wherein T represents a group represented by T6, R^{3b} represents a chlorine atom, R^{3c} represents a hydrogen atom, and R¹ represents any substituents indicated in [Table L1] to [Table L3](hereinafter, referred to as Compound Class SX₄₀₂).

A compound (L-3) wherein T represents a group represented by T6, R^{3b} represents a bromine atom, R^{3c} represents a hydrogen atom, and R¹ represents any substituents indicated in [Table L1] to [Table L3](hereinafter, referred to as Compound Class SX₄₀₃).

A compound (L-3) wherein T represents a group represented by T6, R^{3b} represents an iodine atom, R^{3c} represents a hydrogen atom, and R¹ represents any substituents indicated in [Table L1] to [Table L3](hereinafter, referred to as Compound Class SX₄₀₄).

A compound (L-3) wherein T represents a group represented by T6, R^{3b} represents a hydrogen atom, R^{3c} represents CF₃, and R¹ represents any substituents indicated in [Table L1] to [Table L3](hereinafter, referred to as Compound Class SX₄₀₅).

A compound (L-3) wherein T represents a group represented by T6, R^{3b} represents a hydrogen atom, R^{3c} represents a chlorine atom, and R¹ represents any substituents indicated in [Table L1] to [Table L3](hereinafter, referred to as Compound Class SX₄₀₆).

A compound (L-3) wherein T represents a group represented by T6, R^{3b} represents a hydrogen atom, R^{3c} represents a bromine atom and R¹ represents any substituents indicated in [Table L1] to [Table L3](hereinafter, referred to as Compound Class SX₄₀₇).

A compound (L-3) wherein T represents a group represented by T6, R^{3b} represents a hydrogen atom, R^{3c} represents an iodine atom and R¹ represents any substituents indicated in [Table L1] to [Table L3](hereinafter, referred to as Compound Class SX₄₀₈).

A compound (L-3) wherein T represents a group represented by T7, R^{3b} represents a hydrogen atom, R^{3c} represents a hydrogen atom, and R¹ represents any substituents indicated in [Table L1] to [Table L3](hereinafter, referred to as Compound Class SX₄₀₉).

A compound (L-3) wherein T represents a group represented by T7, R^{3b} represents CF₃, R^{3c} represents a hydrogen atom, and R¹ represents any substituents indicated in [Table L1] to [Table L3](hereinafter, referred to as Compound Class SX₄₁₀).

A compound (L-3) wherein T represents a group represented by T7, R^{3b} represents a chlorine atom, R^{3c} represents a hydrogen atom, and R¹ represents any substituents indicated in [Table L1] to [Table L3](hereinafter, referred to as Compound Class SX₄₁₁).

A compound (L-3) wherein T represents a group represented by T7, R^{3b} represents a bromine atom, R^{3c} represents a hydrogen atom, and R¹ represents any substituents indicated in [Table L1] to [Table L3](hereinafter, referred to as Compound Class SX₄₁₂).

A compound (L-3) wherein T represents a group represented by T7, R^{3b} represents an iodine atom, R^{3c} represents a hydrogen atom, and R¹ represents any substituents indicated in [Table L1] to [Table L3](hereinafter, referred to as Compound Class SX₄₁₃).

A compound (L-3) wherein T represents a group represented by T7, R^{3b} represents a hydrogen atom, R^{3c} represents CF₃, and R¹ represents any substituents indicated in [Table L1] to [Table L3](hereinafter, referred to as Compound Class SX₄₁₄).

A compound (L-3) wherein T represents a group represented by T7, R^{3b} represents a hydrogen atom, R^{3c} represents a chlorine atom, and R¹ represents any substituents indicated in [Table L1] to [Table L3](hereinafter, referred to as Compound Class SX₄₁₅).

A compound (L-3) wherein T represents a group represented by T7, R^{3b} represents a hydrogen atom, R^{3c} represents a bromine atom and R¹ represents any substituents indicated in [Table L1] to [Table L3](hereinafter, referred to as Compound Class SX₄₁₆).

A compound (L-3) wherein T represents a group represented by T7, R^{3b} represents a hydrogen atom, R^{3c} represents an iodine atom and R¹ represents any substituents indicated in [Table L1] to [Table L3](hereinafter, referred to as Compound Class SX₄₁₇).

A compound (L-3) wherein T represents a group represented by T8, R^{3b} represents a hydrogen atom, R^{3c} represents a hydrogen atom, and R¹ represents any substituents indicated in [Table L1] to [Table L3](hereinafter, referred to as Compound Class SX₄₁₈).

A compound (L-3) wherein T represents a group represented by T8, R^{3b} represents CF₃, R^{3c} represents a hydrogen atom, and R¹ represents any substituents indicated in [Table L1] to [Table L3](hereinafter, referred to as Compound Class SX₄₁₉).

A compound (L-3) wherein T represents a group represented by T8, R^{3b} represents a chlorine atom, R^{3c} represents a hydrogen atom, and R¹ represents any substituents indicated in [Table L1] to [Table L3](hereinafter, referred to as Compound Class SX₄₂₀).

A compound (L-3) wherein T represents a group represented by T8, R^{3b} represents a bromine atom, R^{3c} represents a hydrogen atom, and R¹ represents any substituents indicated in [Table L1] to [Table L3](hereinafter, referred to as Compound Class SX₄₂₁).

A compound (L-3) wherein T represents a group represented by T8, R^{3b} represents an iodine atom, R^{3c} represents a hydrogen atom, and R¹ represents any substituents indicated in [Table L1] to [Table L3](hereinafter, referred to as Compound Class SX₄₂₂).

A compound (L-3) wherein T represents a group represented by T8, R^{3b} represents a hydrogen atom, R^{3c} represents CF₃, and R¹ represents any substituents indicated in [Table L1] to [Table L3](hereinafter, referred to as Compound Class SX₄₂₃).

A compound (L-3) wherein T represents a group represented by T8, R^{3b} represents a hydrogen atom, R^{3c} represents a chlorine atom, and R¹ represents any substituents indicated in [Table L1] to [Table L3](hereinafter, referred to as Compound Class SX₄₂₄).

A compound (L-3) wherein T represents a group represented by T8, R^{3b} represents a hydrogen atom, R^{3c} represents a bromine atom and R¹ represents any substituents indicated in [Table L1] to [Table L3](hereinafter, referred to as Compound Class SX₄₂₅).

A compound (L-3) wherein T represents a group represented by T8, R^{3b} represents a hydrogen atom, R^{3c} represents an iodine atom and R¹ represents any substituents indicated in [Table L1] to [Table L3](hereinafter, referred to as Compound Class SX₄₂₆).

A compound (L-3) wherein T represents a group represented by T9, R^{3b} represents a hydrogen atom, R^{3c} represents a hydrogen atom, and R¹ represents any substituents indicated in [Table L1] to [Table L3](hereinafter, referred to as Compound Class SX₄₂₇).

A compound (L-3) wherein T represents a group represented by T9, R^{3b} represents CF₃, R^{3c} represents a hydrogen atom, and R¹ represents any substituents indicated in [Table L1] to [Table L3](hereinafter, referred to as Compound Class SX₄₂₈).

A compound (L-3) wherein T represents a group represented by T9, R^{3b} represents a chlorine atom, R^{3c} represents a hydrogen atom, and R¹ represents any substituents indicated in [Table L1] to [Table L3](hereinafter, referred to as Compound Class SX₄₂₉).

A compound (L-3) wherein T represents a group represented by T9, R^{3b} represents a bromine atom, R^{3c} represents a hydrogen atom, and R¹ represents any substituents indicated in [Table L1] to [Table L3](hereinafter, referred to as Compound Class SX₄₃₀).

A compound (L-3) wherein T represents a group represented by T9, R^{3b} represents an iodine atom, R^{3c} represents a hydrogen atom, and R¹ represents any substituents indicated in [Table L1] to [Table L3](hereinafter, referred to as Compound Class SX₄₃₁).

A compound (L-3) wherein T represents a group represented by T9, R^{3b} represents a hydrogen atom, R^{3c} represents CF₃, and R¹ represents any substituents indicated in [Table L1] to [Table L3](hereinafter, referred to as Compound Class SX₄₃₂).

A compound (L-3) wherein T represents a group represented by T9, R^{3b} represents a hydrogen atom, R^{3c} represents a chlorine atom, and R¹ represents any substituents indicated in [Table L1] to [Table L3](hereinafter, referred to as Compound Class SX₄₃₃).

A compound (L-3) wherein T represents a group represented by T9, R^{3b} represents a hydrogen atom, R^{3c} represents a bromine atom and R¹ represents any substituents indicated in [Table L1] to [Table L3](hereinafter, referred to as Compound Class SX₄₃₄).

A compound (L-3) wherein T represents a group represented by T9, R^{3b} represents a hydrogen atom, R^{3c} represents an iodine atom and R¹ represents any substituents indicated in [Table L1] to [Table L3](hereinafter, referred to as Compound Class SX₄₃₅).

A compound (L-3) wherein T represents a group represented by T10, R^{3b} represents a hydrogen atom, R^{3c} represents a hydrogen atom, and R¹ represents any substituents indicated in [Table L1] to [Table L3](hereinafter, referred to as Compound Class SX₄₃₆).

A compound (L-3) wherein T represents a group represented by T10, R^{3b} represents CF₃, R^{3c} represents a hydrogen atom, and R¹ represents any substituents indicated in [Table L1] to [Table L3](hereinafter, referred to as Compound Class SX₄₃₇).

A compound (L-3) wherein T represents a group represented by T10, R^{3b} represents a chlorine atom, R^{3c} represents a hydrogen atom, and R¹ represents any substituents indicated in [Table L1] to [Table L3](hereinafter, referred to as Compound Class SX₄₃₈).

A compound (L-3) wherein T represents a group represented by T10, R^{3b} represents a bromine atom, R^{3c} represents a hydrogen atom, and R¹ represents any substituents indicated in [Table L1] to [Table L3](hereinafter, referred to as Compound Class SX₄₃₉).

A compound (L-3) wherein T represents a group represented by T10, R^{3b} represents an iodine atom, R^{3c} represents a hydrogen atom, and R¹ represents any substituents indicated in [Table L1] to [Table L3](hereinafter, referred to as Compound Class SX₄₄₀).

A compound (L-3) wherein T represents a group represented by T10, R^{3b} represents a hydrogen atom, R^{3c} represents CF₃, and R¹ represents any substituents indicated in [Table L1] to [Table L3](hereinafter, referred to as Compound Class SX₄₄₁).

A compound (L-3) wherein T represents a group represented by T10, R^{3b} represents a hydrogen atom, R^{3c} represents a chlorine atom, and R¹ represents any substituents indicated in [Table L1] to [Table L3](hereinafter, referred to as Compound Class SX₄₄₂).

A compound (L-3) wherein T represents a group represented by T10, R^{3b} represents a hydrogen atom, R^{3c} represents a bromine atom and R¹ represents any substituents indicated in [Table L1] to [Table L3](hereinafter, referred to as Compound Class SX₄₄₃).

A compound (L-3) wherein T represents a group represented by T10, R^{3b} represents a hydrogen atom, R^{3c} represents an iodine atom and R¹ represents any substituents indicated in [Table L1] to [Table L3](hereinafter, referred to as Compound Class SX₄₄₄).

A compound (L-3) wherein T represents a group represented by T1l, R^{3b} represents a hydrogen atom, R^{3c} represents a hydrogen atom, and R¹ represents any substituents indicated in [Table L1] to [Table L3](hereinafter, referred to as Compound Class SX₄₄₅).

A compound (L-3) wherein T represents a group represented by T1l, R^{3b} represents CF₃, R^{3c} represents a hydrogen atom, and R¹ represents any substituents indicated in [Table L1] to [Table L3](hereinafter, referred to as Compound Class SX₄₄₆).

A compound (L-3) wherein T represents a group represented by T1l, R^{3b} represents a chlorine atom, R^{3c} represents a hydrogen atom, and R¹ represents any substituents indicated in [Table L1] to [Table L3](hereinafter, referred to as Compound Class SX₄₄₇).

A compound (L-3) wherein T represents a group represented by T1l, R^{3b} represents a bromine atom, R^{3c} represents a hydrogen atom, and R¹ represents any substituents indicated in [Table L1] to [Table L3](hereinafter, referred to as Compound Class SX₄₄₈).

A compound (L-3) wherein T represents a group represented by T1l, R^{3b} represents an iodine atom, R^{3c} represents a hydrogen atom, and R¹ represents any substituents indicated in [Table L1] to [Table L3](hereinafter, referred to as Compound Class SX₄₄₉).

A compound (L-3) wherein T represents a group represented by T1l, R^{3b} represents a hydrogen atom, R^{3c} represents CF₃, and R¹ represents any substituents indicated in [Table L1] to [Table L3](hereinafter, referred to as Compound Class SX₄₅₀).

A compound (L-3) wherein T represents a group represented by T1l, R^{3b} represents a hydrogen atom, R^{3c} represents a chlorine atom, and R¹ represents any substituents indicated in [Table L1] to [Table L3](hereinafter, referred to as Compound Class SX₄₅₁).

A compound (L-3) wherein T represents a group represented by T1l, R^{3b} represents a hydrogen atom, R^{3c} represents a bromine atom and R¹ represents any substituents indicated in [Table L1] to [Table L3](hereinafter, referred to as Compound Class SX₄₅₂).

A compound (L-3) wherein T represents a group represented by T1l, R^{3b} represents a hydrogen atom, R^{3c} represents an iodine atom and R¹ represents any substituents indicated in [Table L1] to [Table L3](hereinafter, referred to as Compound Class SX₄₅₃).

A compound (L-3) wherein T represents a group represented by T12, R^{3b} represents a hydrogen atom, R^{3c} represents a hydrogen atom, and R¹ represents any substituents indicated in [Table L1] to [Table L3](hereinafter, referred to as Compound Class SX₄₅₄).

A compound (L-3) wherein T represents a group represented by T12, R^{3b} represents CF₃, R^{3c} represents a hydrogen atom, and R¹ represents any substituents indicated in [Table L1] to [Table L3](hereinafter, referred to as Compound Class SX₄₅₅).

A compound (L-3) wherein T represents a group represented by T12, R^{3b} represents a chlorine atom, R^{3c} represents a hydrogen atom, and R¹ represents any substituents indicated in [Table L1] to [Table L3](hereinafter, referred to as Compound Class SX₄₅₆).

A compound (L-3) wherein T represents a group represented by T12, R^{3b} represents a bromine atom, R^{3c} represents a hydrogen atom, and R¹ represents any substituents indicated in [Table L1] to [Table L3](hereinafter, referred to as Compound Class SX₄₅₇).

A compound (L-3) wherein T represents a group represented by T12, R^{3b} represents an iodine atom, R^{3c} represents a hydrogen atom, and R¹ represents any substituents indicated in [Table L1] to [Table L3](hereinafter, referred to as Compound Class SX₄₅₈).

A compound (L-3) wherein T represents a group represented by T12, R^{3b} represents a hydrogen atom, R^{3c} represents CF₃, and R¹ represents any substituents indicated in [Table L1] to [Table L3](hereinafter, referred to as Compound Class SX₄₅₉).

A compound (L-3) wherein T represents a group represented by T12, R^{3b} represents a hydrogen atom, R^{3c} represents a chlorine atom, and R¹ represents any substituents indicated in [Table L1] to [Table L3](hereinafter, referred to as Compound Class SX₄₆₀).

A compound (L-3) wherein T represents a group represented by T12, R^{3b} represents a hydrogen atom, R^{3c} represents a bromine atom and R¹ represents any substituents indicated in [Table L1] to [Table L3](hereinafter, referred to as Compound Class SX₄₆₁).

A compound (L-3) wherein T represents a group represented by T12, R^{3b} represents a hydrogen atom, R^{3c} represents an iodine atom and R¹ represents any substituents indicated in [Table L1] to [Table L3](hereinafter, referred to as Compound Class SX₄₆₂).

A compound (L-3) wherein T represents a group represented by T13, R^{3b} represents a hydrogen atom, R^{3c} represents a hydrogen atom, and R¹ represents any substituents indicated in [Table L1] to [Table L3](hereinafter, referred to as Compound Class SX₄₆₃).

A compound (L-3) wherein T represents a group represented by T13, R^{3b} represents CF₃, R^{3c} represents a hydrogen atom, and R¹ represents any substituents indicated in [Table L1] to [Table L3](hereinafter, referred to as Compound Class SX₄₆₄).

A compound (L-3) wherein T represents a group represented by T13, R^{3b} represents a chlorine atom, R^{3c} represents a hydrogen atom, and R¹ represents any substituents indicated in [Table L1] to [Table L3](hereinafter, referred to as Compound Class SX₄₆₅).

A compound (L-3) wherein T represents a group represented by T13, R^{3b} represents a bromine atom, R^{3c} represents a hydrogen atom, and R¹ represents any substituents indicated in [Table L1] to [Table L3](hereinafter, referred to as Compound Class SX₄₆₆).

A compound (L-3) wherein T represents a group represented by T13, R^{3b} represents an iodine atom, R^{3c} represents a hydrogen atom, and R¹ represents any substituents indicated in [Table L1] to [Table L3](hereinafter, referred to as Compound Class SX₄₆₇).

A compound (L-3) wherein T represents a group represented by T13, R^{3b} represents a hydrogen atom, R^{3c} represents CF₃, and R¹ represents any substituents indicated in [Table L1] to [Table L3](hereinafter, referred to as Compound Class SX₄₆₈).

A compound (L-3) wherein T represents a group represented by T13, R^{3b} represents a hydrogen atom, R^{3c} represents a chlorine atom, and R¹ represents any substituents indicated in [Table L1] to [Table L3](hereinafter, referred to as Compound Class SX₄₆₉).

A compound (L-3) wherein T represents a group represented by T13, R^{3b} represents a hydrogen atom, R^{3c} represents a bromine atom and R¹ represents any substituents indicated in [Table L1] to [Table L3](hereinafter, referred to as Compound Class SX₄₇₀).

A compound (L-3) wherein T represents a group represented by T13, R^{3b} represents a hydrogen atom, R^{3c} represents an iodine atom and R¹ represents any substituents indicated in [Table L1] to [Table L3](hereinafter, referred to as Compound Class SX₄₇₁).

A compound (L-3) wherein T represents a group represented by T14, R^{3b} represents a hydrogen atom, R^{3c} represents a hydrogen atom, and R¹ represents any substituents indicated in [Table L1] to [Table L3](hereinafter, referred to as Compound Class SX₄₇₂).

A compound (L-3) wherein T represents a group represented by T14, R^{3b} represents CF₃, R^{3c} represents a hydrogen atom, and R¹ represents any substituents indicated in [Table L1] to [Table L3](hereinafter, referred to as Compound Class SX₄₇₃).

A compound (L-3) wherein T represents a group represented by T14, R^{3b} represents a chlorine atom, R^{3c} represents a hydrogen atom, and R¹ represents any substituents indicated in [Table L1] to [Table L3](hereinafter, referred to as Compound Class SX₄₇₄).

A compound (L-3) wherein T represents a group represented by T14, R^{3b} represents a bromine atom, R^{3c} represents a hydrogen atom, and R¹ represents any substituents indicated in [Table L1] to [Table L3](hereinafter, referred to as Compound Class SX₄₇₅).

A compound (L-3) wherein T represents a group represented by T14, R^{3b} represents an iodine atom, R^{3c} represents a hydrogen atom, and R¹ represents any substituents indicated in [Table L1] to [Table L3](hereinafter, referred to as Compound Class SX₄₇₆).

A compound (L-3) wherein T represents a group represented by T14, R^{3b} represents a hydrogen atom, R^{3c} represents CF₃, and R¹ represents any substituents indicated in [Table L1] to [Table L3](hereinafter, referred to as Compound Class SX₄₇₇).

A compound (L-3) wherein T represents a group represented by T14, R^{3b} represents a hydrogen atom, R^{3c} represents a chlorine atom, and R¹ represents any substituents indicated in [Table L1] to [Table L3](hereinafter, referred to as Compound Class SX₄₇₈).

A compound (L-3) wherein T represents a group represented by T14, R^{3b} represents a hydrogen atom, R^{3c} represents a bromine atom and R¹ represents any substituents indicated in [Table L1] to [Table L3](hereinafter, referred to as Compound Class SX₄₇₉).

A compound (L-3) wherein T represents a group represented by T14, R^{3b} represents a hydrogen atom, R^{3c} represents an iodine atom and R¹ represents any substituents indicated in [Table L1] to [Table L3](hereinafter, referred to as Compound Class SX₄₈₀).

A compound (L-3) wherein T represents a group represented by T15, R^{3b} represents a hydrogen atom, R^{3c} represents a hydrogen atom, and R¹ represents any substituents indicated in [Table L1] to [Table L3](hereinafter, referred to as Compound Class SX₄₈₁).

A compound (L-3) wherein T represents a group represented by T15, R^{3b} represents CF₃, R^{3c} represents a hydrogen atom, and R¹ represents any substituents indicated in [Table L1] to [Table L3](hereinafter, referred to as Compound Class SX₄₈₂).

A compound (L-3) wherein T represents a group represented by T15, R^{3b} represents a chlorine atom, R^{3c} represents a hydrogen atom, and R¹ represents any substituents indicated in [Table L1] to [Table L3](hereinafter, referred to as Compound Class SX₄₈₃).

A compound (L-3) wherein T represents a group represented by T15, R^{3b} represents a bromine atom, R^{3c} represents a hydrogen atom, and R¹ represents any substituents indicated in [Table L1] to [Table L3](hereinafter, referred to as Compound Class SX₄₈₄).

A compound (L-3) wherein T represents a group represented by T15, R^{3b} represents an iodine atom, R^{3c} represents a hydrogen atom, and R¹ represents any substituents indicated in [Table L1] to [Table L3](hereinafter, referred to as Compound Class SX₄₈₅).

A compound (L-3) wherein T represents a group represented by T15, R^{3b} represents a hydrogen atom, R^{3c} represents CF₃, and R¹ represents any substituents indicated in [Table L1] to [Table L3](hereinafter, referred to as Compound Class SX₄₈₆).

A compound (L-3) wherein T represents a group represented by T15, R^{3b} represents a hydrogen atom, R^{3c} represents a chlorine atom, and R¹ represents any substituents indicated in [Table L1] to [Table L3](hereinafter, referred to as Compound Class SX₄₈₇).

A compound (L-3) wherein T represents a group represented by T15, R^{3b} represents a hydrogen atom, R^{3c} represents a bromine atom and R¹ represents any substituents indicated in [Table L1] to [Table L3](hereinafter, referred to as Compound Class SX₄₈₈).

A compound (L-3) wherein T represents a group represented by T15, R^{3b} represents a hydrogen atom, R^{3c} represents an iodine atom and R¹ represents any substituents indicated in [Table L1] to [Table L3](hereinafter, referred to as Compound Class SX₄₈₉).

A compound (L-3) wherein T represents a group represented by T16, R^{3b} represents a hydrogen atom, R^{3c} represents a hydrogen atom, and R¹ represents any substituents indicated in [Table L1] to [Table L3](hereinafter, referred to as Compound Class SX₄₉₀).

A compound (L-3) wherein T represents a group represented by T16, R^{3b} represents CF₃, R^{3c} represents a hydrogen atom, and R¹ represents any substituents indicated in [Table L1] to [Table L3](hereinafter, referred to as Compound Class SX₄₉₁).

A compound (L-3) wherein T represents a group represented by T16, R^{3b} represents a chlorine atom, R^{3c} represents a hydrogen atom, and R¹ represents any substituents indicated in [Table L1] to [Table L3](hereinafter, referred to as Compound Class SX₄₉₂).

A compound (L-3) wherein T represents a group represented by T16, R^{3b} represents a bromine atom, R^{3c} represents a hydrogen atom, and R¹ represents any substituents indicated in [Table L1] to [Table L3](hereinafter, referred to as Compound Class SX₄₉₃).

A compound (L-3) wherein T represents a group represented by T16, R^{3b} represents an iodine atom, R^{3c} represents a hydrogen atom, and R¹ represents any substituents indicated in [Table L1] to [Table L3](hereinafter, referred to as Compound Class SX₄₉₄).

A compound (L-3) wherein T represents a group represented by T16, R^{3b} represents a hydrogen atom, R^{3c} represents CF₃, and R¹ represents any substituents indicated in [Table L1] to [Table L3](hereinafter, referred to as Compound Class SX₄₉₅).

A compound (L-3) wherein T represents a group represented by T16, R^{3b} represents a hydrogen atom, R^{3c} represents a chlorine atom, and R¹ represents any substituents indicated in [Table L1] to [Table L3](hereinafter, referred to as Compound Class SX₄₉₆).

A compound (L-3) wherein T represents a group represented by T16, R^{3b} represents a hydrogen atom, R^{3c} represents a bromine atom and R¹ represents any substituents indicated in [Table L1] to [Table L3](hereinafter, referred to as Compound Class SX₄₉₇).

A compound (L-3) wherein T represents a group represented by T16, R^{3b} represents a hydrogen atom, R^{3c} represents an iodine atom and R¹ represents any substituents indicated in [Table L1] to [Table L3](hereinafter, referred to as Compound Class SX₄₉₈).

A compound (L-3) wherein T represents a group represented by T17, R^{3b} represents a hydrogen atom, R^{3c} represents a hydrogen atom, and R¹ represents any substituents indicated in [Table L1] to [Table L3](hereinafter, referred to as Compound Class SX₄₉₉).

A compound (L-3) wherein T represents a group represented by T17, R^{3b} represents CF₃, R^{3c} represents a hydrogen atom, and R¹ represents any substituents indicated in [Table L1] to [Table L3](hereinafter, referred to as Compound Class SX₅₀₀).

A compound (L-3) wherein T represents a group represented by T17, R^{3b} represents a chlorine atom, R^{3c} represents a hydrogen atom, and R¹ represents any substituents indicated in [Table L1] to [Table L3](hereinafter, referred to as Compound Class SX₅₀₁).

A compound (L-3) wherein T represents a group represented by T17, R^{3b} represents a bromine atom, R^{3c} represents a hydrogen atom, and R¹ represents any substituents indicated in [Table L1] to [Table L3](hereinafter, referred to as Compound Class SX₅₀₂).

A compound (L-3) wherein T represents a group represented by T17, R^{3b} represents an iodine atom, R^{3c} represents a hydrogen atom, and R¹ represents any substituents indicated in [Table L1] to [Table L3](hereinafter, referred to as Compound Class SX₅₀₃).

A compound (L-3) wherein T represents a group represented by T17, R^{3b} represents a hydrogen atom, R^{3c} represents CF₃, and R¹ represents any substituents indicated in [Table L1] to [Table L3](hereinafter, referred to as Compound Class SX₅₀₄).

A compound (L-3) wherein T represents a group represented by T17, R^{3b} represents a hydrogen atom, R^{3c} represents a chlorine atom, and R¹ represents any substituents indicated in [Table L1] to [Table L3](hereinafter, referred to as Compound Class SX₅₀₅).

A compound (L-3) wherein T represents a group represented by T17, R^{3b} represents a hydrogen atom, R^{3c} represents a bromine atom and R¹ represents any substituents indicated in [Table L1] to [Table L3](hereinafter, referred to as Compound Class SX₅₀₆).

A compound (L-3) wherein T represents a group represented by T17, R^{3b} represents a hydrogen atom, R^{3c} represents an iodine atom and R¹ represents any substituents indicated in [Table L1] to [Table L3](hereinafter, referred to as Compound Class SX₅₀₇).

A compound (L-3) wherein T represents a group represented by T18, R^{3b} represents a hydrogen atom, R^{3c} represents a hydrogen atom, and R¹ represents any substituents indicated in [Table L1] to [Table L3](hereinafter, referred to as Compound Class SX₅₀₈).

A compound (L-3) wherein T represents a group represented by T18, R^{3b} represents CF₃, R^{3c} represents a hydrogen atom, and R¹ represents any substituents indicated in [Table L1] to [Table L3](hereinafter, referred to as Compound Class SX₅₀₉).

A compound (L-3) wherein T represents a group represented by T18, R^{3b} represents a chlorine atom, R^{3c} represents a hydrogen atom, and R¹ represents any substituents indicated in [Table L1] to [Table L3](hereinafter, referred to as Compound Class SX₅₁₀).

A compound (L-3) wherein T represents a group represented by T18, R^{3b} represents a bromine atom, R^{3c} represents a hydrogen atom, and R¹ represents any substituents indicated in [Table L1] to [Table L3](hereinafter, referred to as Compound Class SX₅₁₁).

A compound (L-3) wherein T represents a group represented by T18, R^{3b} represents an iodine atom, R^{3c} represents a hydrogen atom, and R¹ represents any substituents indicated in [Table L1] to [Table L3](hereinafter, referred to as Compound Class SX₅₁₂).

A compound (L-3) wherein T represents a group represented by T18, R^{3b} represents a hydrogen atom, R^{3c} represents CF₃, and R¹ represents any substituents indicated in [Table L1] to [Table L3](hereinafter, referred to as Compound Class SX₅₁₃).

A compound (L-3) wherein T represents a group represented by T18, R^{3b} represents a hydrogen atom, R^{3c} represents a chlorine atom, and R¹ represents any substituents indicated in [Table L1] to [Table L3](hereinafter, referred to as Compound Class SX₅₁₄).

A compound (L-3) wherein T represents a group represented by T18, R^{3b} represents a hydrogen atom, R^{3c} represents a bromine atom and R¹ represents any substituents indicated in [Table L1] to [Table L3](hereinafter, referred to as Compound Class SX₅₁₅).

A compound (L-3) wherein T represents a group represented by T18, R^{3b} represents a hydrogen atom, R^{3c} represents an iodine atom and R¹ represents any substituents indicated in [Table L1] to [Table L3](hereinafter, referred to as Compound Class SX₅₁₆).

A compound (L-3) wherein T represents a group represented by T19, R^{3b} represents a hydrogen atom, R^{3c} represents a hydrogen atom, and R¹ represents any substituents indicated in [Table L1] to [Table L3](hereinafter, referred to as Compound Class SX₅₁₇).

A compound (L-3) wherein T represents a group represented by T19, R^{3b} represents CF₃, R^{3c} represents a hydrogen atom, and R¹ represents any substituents indicated in [Table L1] to [Table L3](hereinafter, referred to as Compound Class SX₅₁₈).

A compound (L-3) wherein T represents a group represented by T19, R^{3b} represents a chlorine atom, R^{3c} represents a hydrogen atom, and R¹ represents any substituents indicated in [Table L1] to [Table L3](hereinafter, referred to as Compound Class SX₅₁₉).

A compound (L-3) wherein T represents a group represented by T19, R^{3b} represents a bromine atom, R^{3c} represents a hydrogen atom, and R¹ represents any substituents indicated in [Table L1] to [Table L3](hereinafter, referred to as Compound Class SX₅₂₀).

A compound (L-3) wherein T represents a group represented by T19, R^{3b} represents an iodine atom, R^{3c} represents a hydrogen atom, and R¹ represents any substituents indicated in [Table L1] to [Table L3](hereinafter, referred to as Compound Class SX₅₂₁).

A compound (L-3) wherein T represents a group represented by T19, R^{3b} represents a hydrogen atom, R^{3c} represents CF₃, and R¹ represents any substituents indicated in [Table L1] to [Table L3](hereinafter, referred to as Compound Class SX₅₂₂).

A compound (L-3) wherein T represents a group represented by T19, R^{3b} represents a hydrogen atom, R^{3c} represents a chlorine atom, and R¹ represents any substituents indicated in [Table L1] to [Table L3](hereinafter, referred to as Compound Class SX₅₂₃).

A compound (L-3) wherein T represents a group represented by T19, R^{3b} represents a hydrogen atom, R^{3c} represents a bromine atom and R¹ represents any substituents indicated in [Table L1] to [Table L3](hereinafter, referred to as Compound Class SX₅₂₄).

A compound (L-3) wherein T represents a group represented by T19, R^{3b} represents a hydrogen atom, R^{3c} represents an iodine atom and R¹ represents any substituents indicated in [Table L1] to [Table L3](hereinafter, referred to as Compound Class SX₅₂₅).

A compound (L-3) wherein T represents a group represented by T20, R^{3b} represents a hydrogen atom, R^{3c} represents a hydrogen atom, and R¹ represents any substituents indicated in [Table L1] to [Table L3](hereinafter, referred to as Compound Class SX₅₂₆).

A compound (L-3) wherein T represents a group represented by T20, R^{3b} represents CF₃, R^{3c} represents a hydrogen atom, and R¹ represents any substituents indicated in [Table L1] to [Table L3](hereinafter, referred to as Compound Class SX₅₂₇).

A compound (L-3) wherein T represents a group represented by T20, R^{3b} represents a chlorine atom, R^{3c} represents a hydrogen atom, and R¹ represents any substituents indicated in [Table L1] to [Table L3](hereinafter, referred to as Compound Class SX₅₂₈).

A compound (L-3) wherein T represents a group represented by T20, R^{3b} represents a bromine atom, R^{3c} represents a hydrogen atom, and R¹ represents any substituents indicated in [Table L1] to [Table L3](hereinafter, referred to as Compound Class SX₅₂₉).

A compound (L-3) wherein T represents a group represented by T20, R^{3b} represents an iodine atom, R^{3c} represents a hydrogen atom, and R¹ represents any substituents indicated in [Table L1] to [Table L3](hereinafter, referred to as Compound Class SX₅₃₀).

A compound (L-3) wherein T represents a group represented by T20, R^{3b} represents a hydrogen atom, R^{3c} represents CF₃, and R¹ represents any substituents indicated in [Table L1] to [Table L3](hereinafter, referred to as Compound Class SX₅₃₁).

A compound (L-3) wherein T represents a group represented by T20, R^{3b} represents a hydrogen atom, R^{3c} represents a chlorine atom, and R¹ represents any substituents indicated in [Table L1] to [Table L3](hereinafter, referred to as Compound Class SX₅₃₂).

A compound (L-3) wherein T represents a group represented by T20, R^{3b} represents a hydrogen atom, R^{3c} represents a bromine atom and R¹ represents any substituents indicated in [Table L1] to [Table L3](hereinafter, referred to as Compound Class SX₅₃₃).

A compound (L-3) wherein T represents a group represented by T20, R^{3b} represents a hydrogen atom, R^{3c} represents an iodine atom and R¹ represents any substituents indicated in [Table L1] to [Table L3](hereinafter, referred to as Compound Class SX₅₃₄).

A compound (L-3) wherein T represents a group represented by T21, R^{3b} represents a hydrogen atom, R^{3c} represents a hydrogen atom, and R¹ represents any substituents indicated in [Table L1] to [Table L3](hereinafter, referred to as Compound Class SX₅₃₅).

A compound (L-3) wherein T represents a group represented by T21, R^{3b} represents CF₃, R^{3c} represents a hydrogen atom, and R¹ represents any substituents indicated in [Table L1] to [Table L3](hereinafter, referred to as Compound Class SX₅₃₆).

A compound (L-3) wherein T represents a group represented by T21, R^{3b} represents a chlorine atom, R^{3c} represents a hydrogen atom, and R¹ represents any substituents indicated in [Table L1] to [Table L3](hereinafter, referred to as Compound Class SX₅₃₇).

A compound (L-3) wherein T represents a group represented by T21, R^{3b} represents a bromine atom, R^{3c} represents a hydrogen atom, and R¹ represents any substituents indicated in [Table L1] to [Table L3](hereinafter, referred to as Compound Class SX₅₃₈).

A compound (L-3) wherein T represents a group represented by T21, R^{3b} represents an iodine atom, R^{3c} represents a hydrogen atom, and R¹ represents any substituents indicated in [Table L1] to [Table L3](hereinafter, referred to as Compound Class SX₅₃₉).

A compound (L-3) wherein T represents a group represented by T21, R^{3b} represents a hydrogen atom, R^{3c} represents CF₃, and R¹ represents any substituents indicated in [Table L1] to [Table L3](hereinafter, referred to as Compound Class SX₅₄₀).

A compound (L-3) wherein T represents a group represented by T21, R^{3b} represents a hydrogen atom, R^{3c} represents a chlorine atom, and R¹ represents any substituents indicated in [Table L1] to [Table L3](hereinafter, referred to as Compound Class SX₅41).

A compound (L-3) wherein T represents a group represented by T21, R^{3b} represents a hydrogen atom, R^{3c} represents a bromine atom and R¹ represents any substituents indicated in [Table L1] to [Table L3](hereinafter, referred to as Compound Class SX₅₄₂).

A compound (L-3) wherein T represents a group represented by T21, R^{3b} represents a hydrogen atom, R^{3c} represents an iodine atom and R¹ represents any substituents indicated in [Table L1] to [Table L3](hereinafter, referred to as Compound Class SX₅₄₃).

A compound (L-3) wherein T represents a group represented by T22, R^{3b} represents a hydrogen atom, R^{3c} represents a hydrogen atom, and R¹ represents any substituents indicated in [Table L1] to [Table L3](hereinafter, referred to as Compound Class SX₅₄₄).

A compound (L-3) wherein T represents a group represented by T22, R^{3b} represents CF₃, R^{3c} represents a hydrogen atom, and R¹ represents any substituents indicated in [Table L1] to [Table L3](hereinafter, referred to as Compound Class SX₅₄₅).

A compound (L-3) wherein T represents a group represented by T22, R^{3b} represents a chlorine atom, R^{3c} represents a hydrogen atom, and R¹ represents any substituents indicated in [Table L1] to [Table L3](hereinafter, referred to as Compound Class SX₅₄₆).

A compound (L-3) wherein T represents a group represented by T22, R^{3b} represents a bromine atom, R^{3c} represents a hydrogen atom, and R¹ represents any substituents indicated in [Table L1] to [Table L3](hereinafter, referred to as Compound Class SX₅₄₇).

A compound (L-3) wherein T represents a group represented by T22, R^{3b} represents an iodine atom, R^{3c} represents a hydrogen atom, and R¹ represents any substituents indicated in [Table L1] to [Table L3](hereinafter, referred to as Compound Class SX₅₄₈).

A compound (L-3) wherein T represents a group represented by T22, R^{3b} represents a hydrogen atom, R^{3c} represents CF₃, and R¹ represents any substituents indicated in [Table L1] to [Table L3](hereinafter, referred to as Compound Class SX₅₄₉).

A compound (L-3) wherein T represents a group represented by T22, R^{3b} represents a hydrogen atom, R^{3c} represents a chlorine atom, and R¹ represents any substituents indicated in [Table L1] to [Table L3](hereinafter, referred to as Compound Class SX₅₅₀).

A compound (L-3) wherein T represents a group represented by T22, R^{3b} represents a hydrogen atom, R^{3c} represents a bromine atom and R¹ represents any substituents indicated in [Table L1] to [Table L3](hereinafter, referred to as Compound Class SX₅₅₁).

A compound (L-3) wherein T represents a group represented by T22, R^{3b} represents a hydrogen atom, R^{3c} represents an iodine atom and R¹ represents any substituents indicated in [Table L1] to [Table L3](hereinafter, referred to as Compound Class SX₅₅₂).

A compound (L-3) wherein T represents a group represented by T23, R^{3b} represents a hydrogen atom, R^{3c} represents a hydrogen atom, and R¹ represents any substituents indicated in [Table L1] to [Table L3](hereinafter, referred to as Compound Class SX₅₅₃).

A compound (L-3) wherein T represents a group represented by T23, R^{3b} represents CF₃, R^{3c} represents a hydrogen atom, and R¹ represents any substituents indicated in [Table L1] to [Table L3](hereinafter, referred to as Compound Class SX₅₅₄).

A compound (L-3) wherein T represents a group represented by T23, R^{3b} represents a chlorine atom, R^{3c} represents a hydrogen atom, and R¹ represents any substituents indicated in [Table L1] to [Table L3](hereinafter, referred to as Compound Class SX₅₅₅).

A compound (L-3) wherein T represents a group represented by T23, R^{3b} represents a bromine atom, R^{3c} represents a hydrogen atom, and R¹ represents any substituents indicated in [Table L1] to [Table L3](hereinafter, referred to as Compound Class SX₅₅₆).

A compound (L-3) wherein T represents a group represented by T23, R^{3b} represents an iodine atom, R^{3c} represents a hydrogen atom, and R¹ represents any substituents indicated in [Table L1] to [Table L3](hereinafter, referred to as Compound Class SX₅₅₇).

A compound (L-3) wherein T represents a group represented by T23, R^{3b} represents a hydrogen atom, R^{3c} represents CF₃, and R¹ represents any substituents indicated in [Table L1] to [Table L3](hereinafter, referred to as Compound Class SX₅₅₈).

A compound (L-3) wherein T represents a group represented by T23, R^{3b} represents a hydrogen atom, R^{3c} represents a chlorine atom, and R¹ represents any substituents indicated in [Table L1] to [Table L3](hereinafter, referred to as Compound Class SX₅₅₉).

A compound (L-3) wherein T represents a group represented by T23, R^{3b} represents a hydrogen atom, R^{3c} represents a bromine atom and R¹ represents any substituents indicated in [Table L1] to [Table L3](hereinafter, referred to as Compound Class SX₅₆₀).

A compound (L-3) wherein T represents a group represented by T23, R^{3b} represents a hydrogen atom, R^{3c} represents an iodine atom and R¹ represents any substituents indicated in [Table L1] to [Table L3](hereinafter, referred to as Compound Class SX₅₆₁).

A compound (L-3) wherein T represents a group represented by T24, R^{3b} represents a hydrogen atom, R^{3c} represents a hydrogen atom, and R¹ represents any substituents indicated in [Table L1] to [Table L3](hereinafter, referred to as Compound Class SX₅₆₂).

A compound (L-3) wherein T represents a group represented by T24, R^{3b} represents CF₃, R^{3c} represents a hydrogen atom, and R¹ represents any substituents indicated in [Table L1] to [Table L3](hereinafter, referred to as Compound Class SX₅₆₃).

A compound (L-3) wherein T represents a group represented by T24, R^{3b} represents a chlorine atom, R^{3c} represents a hydrogen atom, and R¹ represents any substituents indicated in [Table L1] to [Table L3](hereinafter, referred to as Compound Class SX₅₆₄).

A compound (L-3) wherein T represents a group represented by T24, R^{3b} represents a bromine atom, R^{3c} represents a hydrogen atom, and R¹ represents any substituents indicated in [Table L1] to [Table L3](hereinafter, referred to as Compound Class SX₅₆₅).

A compound (L-3) wherein T represents a group represented by T24, R^{3b} represents an iodine atom, R^{3c} represents a hydrogen atom, and R¹ represents any substituents indicated in [Table L1] to [Table L3](hereinafter, referred to as Compound Class SX₅₆₆).

A compound (L-3) wherein T represents a group represented by T24, R^{3b} represents a hydrogen atom, R^{3c} represents CF₃, and R¹ represents any substituents indicated in [Table L1] to [Table L3](hereinafter, referred to as Compound Class SX₅₆₇).

A compound (L-3) wherein T represents a group represented by T24, R^{3b} represents a hydrogen atom, R^{3c} represents a chlorine atom, and R¹ represents any substituents indicated in [Table L1] to [Table L3](hereinafter, referred to as Compound Class SX₅₆₈).

A compound (L-3) wherein T represents a group represented by T24, R^{3b} represents a hydrogen atom, R^{3c} represents a bromine atom and R¹ represents any substituents indicated in [Table L1] to [Table L3](hereinafter, referred to as Compound Class SX₅₆₉).

A compound (L-3) wherein T represents a group represented by T24, R^{3b} represents a hydrogen atom, R^{3c} represents an iodine atom and R¹ represents any substituents indicated in [Table L1] to [Table L3](hereinafter, referred to as Compound Class SX₅₇₀).

A compound (L-3) wherein T represents a group represented by T25, R^{3b} represents a hydrogen atom, R^{3c} represents a hydrogen atom, and R¹ represents any substituents indicated in [Table L1] to [Table L3](hereinafter, referred to as Compound Class SX₅₇₁).

A compound (L-3) wherein T represents a group represented by T25, R^{3b} represents CF₃, R^{3c} represents a hydrogen atom, and R¹ represents any substituents indicated in [Table L1] to [Table L3](hereinafter, referred to as Compound Class SX₅₇₂).

A compound (L-3) wherein T represents a group represented by T25, R^{3b} represents a chlorine atom, R^{3c} represents a hydrogen atom, and R¹ represents any substituents indicated in [Table L1] to [Table L3](hereinafter, referred to as Compound Class SX₅₇₃).

A compound (L-3) wherein T represents a group represented by T25, R^{3b} represents a bromine atom, R^{3c} represents a hydrogen atom, and R¹ represents any substituents indicated in [Table L1] to [Table L3](hereinafter, referred to as Compound Class SX₅₇₄).

A compound (L-3) wherein T represents a group represented by T25, R^{3b} represents an iodine atom, R^{3c} represents a hydrogen atom, and R¹ represents any substituents indicated in [Table L1] to [Table L3](hereinafter, referred to as Compound Class SX₅₇₅).

A compound (L-3) wherein T represents a group represented by T25, R^{3b} represents a hydrogen atom, R^{3c} represents CF₃, and R¹ represents any substituents indicated in [Table L1] to [Table L3](hereinafter, referred to as Compound Class SX₅₇₆).

A compound (L-3) wherein T represents a group represented by T25, R^{3b} represents a hydrogen atom, R^{3c} represents a chlorine atom, and R¹ represents any substituents indicated in [Table L1] to [Table L3](hereinafter, referred to as Compound Class SX₅₇₇).

A compound (L-3) wherein T represents a group represented by T25, R^{3b} represents a hydrogen atom, R^{3c} represents a bromine atom and R¹ represents any substituents indicated in [Table L1] to [Table L3](hereinafter, referred to as Compound Class SX₅₇₈).

A compound (L-3) wherein T represents a group represented by T25, R^{3b} represents a hydrogen atom, R^{3c} represents an iodine atom and R¹ represents any substituents indicated in [Table L1] to [Table L3](hereinafter, referred to as Compound Class SX₅₇₉).

A compound (L-3) wherein T represents a group represented by T26, R^{3b} represents a hydrogen atom, R^{3c} represents a hydrogen atom, and R¹ represents any substituents indicated in [Table L1] to [Table L3](hereinafter, referred to as Compound Class SX₅₈₀).

A compound (L-3) wherein T represents a group represented by T26, R^{3b} represents CF₃, R^{3c} represents a hydrogen atom, and R¹ represents any substituents indicated in [Table L1] to [Table L3](hereinafter, referred to as Compound Class SX₅₈₁).

A compound (L-3) wherein T represents a group represented by T26, R^{3b} represents a chlorine atom, R^{3c} represents a hydrogen atom, and R¹ represents any substituents indicated in [Table L1] to [Table L3](hereinafter, referred to as Compound Class SX₅₈₂).

A compound (L-3) wherein T represents a group represented by T26, R^{3b} represents a bromine atom, R^{3c} represents a hydrogen atom, and R¹ represents any substituents indicated in [Table L1] to [Table L3](hereinafter, referred to as Compound Class SX₅₈₃).

A compound (L-3) wherein T represents a group represented by T26, R^{3b} represents an iodine atom, R^{3c} represents a hydrogen atom, and R¹ represents any substituents indicated in [Table L1] to [Table L3](hereinafter, referred to as Compound Class SX₅₈₄).

A compound (L-3) wherein T represents a group represented by T26, R^{3b} represents a hydrogen atom, R^{3c} represents CF₃, and R¹ represents any substituents indicated in [Table L1] to [Table L3](hereinafter, referred to as Compound Class SX₅₈₅).

A compound (L-3) wherein T represents a group represented by T26, R^{3b} represents a hydrogen atom, R^{3c} represents a chlorine atom, and R¹ represents any substituents indicated in [Table L1] to [Table L3](hereinafter, referred to as Compound Class SX₅₈₆).

A compound (L-3) wherein T represents a group represented by T26, R^{3b} represents a hydrogen atom, R^{3c} represents a bromine atom and R¹ represents any substituents indicated in [Table L1] to [Table L3](hereinafter, referred to as Compound Class SX₅₈₇).

A compound (L-3) wherein T represents a group represented by T26, R^{3b} represents a hydrogen atom, R^{3c} represents an iodine atom and R¹ represents any substituents indicated in [Table L1] to [Table L3](hereinafter, referred to as Compound Class SX₅₈₈).

A compound (L-3) wherein T represents a group represented by T27, R^{3b} represents a hydrogen atom, R^{3c} represents a hydrogen atom, and R¹ represents any substituents indicated in [Table L1] to [Table L3](hereinafter, referred to as Compound Class SX₅₈₉).

A compound (L-3) wherein T represents a group represented by T27, R^{3b} represents CF₃, R^{3c} represents a hydrogen atom, and R¹ represents any substituents indicated in [Table L1] to [Table L3](hereinafter, referred to as Compound Class SX₅₉₀).

A compound (L-3) wherein T represents a group represented by T27, R^{3b} represents a chlorine atom, R^{3c} represents a hydrogen atom, and R¹ represents any substituents indicated in [Table L1] to [Table L3](hereinafter, referred to as Compound Class SX₅₉₁).

A compound (L-3) wherein T represents a group represented by T27, R^{3b} represents a bromine atom, R^{3c} represents a hydrogen atom, and R¹ represents any substituents indicated in [Table L1] to [Table L3](hereinafter, referred to as Compound Class SX₅₉₂).

A compound (L-3) wherein T represents a group represented by T27, R^{3b} represents an iodine atom, R^{3c} represents a hydrogen atom, and R¹ represents any substituents indicated in [Table L1] to [Table L3](hereinafter, referred to as Compound Class SX₅₉₃).

A compound (L-3) wherein T represents a group represented by T27, R^{3b} represents a hydrogen atom, R^{3c} represents CF₃, and R¹ represents any substituents indicated in [Table L1] to [Table L3](hereinafter, referred to as Compound Class SX₅₉₄).

A compound (L-3) wherein T represents a group represented by T27, R^{3b} represents a hydrogen atom, R^{3c} represents a chlorine atom, and R¹ represents any substituents indicated in [Table L1] to [Table L3](hereinafter, referred to as Compound Class SX₅₉₅).

A compound (L-3) wherein T represents a group represented by T27, R^{3b} represents a hydrogen atom, R^{3c} represents a bromine atom and R¹ represents any substituents indicated in [Table L1] to [Table L3](hereinafter, referred to as Compound Class SX₅₉₆).

A compound (L-3) wherein T represents a group represented by T27, R^{3b} represents a hydrogen atom, R^{3c} represents an iodine atom and R¹ represents any substituents indicated in [Table L1] to [Table L3](hereinafter, referred to as Compound Class SX₅₉₇).

A compound (L-3) wherein T represents a group represented by T28, R^{3b} represents a hydrogen atom, R^{3c} represents a hydrogen atom, and R¹ represents any substituents indicated in [Table L1] to [Table L3](hereinafter, referred to as Compound Class SX₅₉₈).

A compound (L-3) wherein T represents a group represented by T28, R^{3b} represents CF₃, R^{3c} represents a hydrogen atom, and R¹ represents any substituents indicated in [Table L1] to [Table L3](hereinafter, referred to as Compound Class SX₅₉₉).

A compound (L-3) wherein T represents a group represented by T28, R^{3b} represents a chlorine atom, R^{3c} represents a hydrogen atom, and R¹ represents any substituents indicated in [Table L1] to [Table L3](hereinafter, referred to as Compound Class SX₆₀₀).

A compound (L-3) wherein T represents a group represented by T28, R^{3b} represents a bromine atom, R^{3c} represents a hydrogen atom, and R¹ represents any substituents indicated in [Table L1] to [Table L3](hereinafter, referred to as Compound Class SX₆₀₁).

A compound (L-3) wherein T represents a group represented by T28, R^{3b} represents an iodine atom, R^{3c} represents a hydrogen atom, and R¹ represents any substituents indicated in [Table L1] to [Table L3](hereinafter, referred to as Compound Class SX₆₀₂).

A compound (L-3) wherein T represents a group represented by T28, R^{3b} represents a hydrogen atom, R^{3c} represents CF₃, and R¹ represents any substituents indicated in [Table L1] to [Table L3](hereinafter, referred to as Compound Class SX₆₀₃).

A compound (L-3) wherein T represents a group represented by T28, R^{3b} represents a hydrogen atom, R^{3c} represents a chlorine atom, and R¹ represents any substituents indicated in [Table L1] to [Table L3](hereinafter, referred to as Compound Class SX₆₀₄).

A compound (L-3) wherein T represents a group represented by T28, R^{3b} represents a hydrogen atom, R^{3c} represents a bromine atom and R¹ represents any substituents indicated in [Table L1] to [Table L3](hereinafter, referred to as Compound Class SX₆₀₅).

A compound (L-3) wherein T represents a group represented by T28, R^{3b} represents a hydrogen atom, R^{3c} represents an iodine atom and R¹ represents any substituents indicated in [Table L1] to [Table L3](hereinafter, referred to as Compound Class SX₆₀₆).

A compound (L-3) wherein T represents a group represented by T29, R^{3b} represents a hydrogen atom, R^{3c} represents a hydrogen atom, and R¹ represents any substituents indicated in [Table L1] to [Table L3](hereinafter, referred to as Compound Class SX₆₀₇).

A compound (L-3) wherein T represents a group represented by T29, R^{3b} represents CF₃, R^{3c} represents a hydrogen atom, and R¹ represents any substituents indicated in [Table L1] to [Table L3](hereinafter, referred to as Compound Class SX₆₀₈).

A compound (L-3) wherein T represents a group represented by T29, R^{3b} represents a chlorine atom, R^{3c} represents a hydrogen atom, and R¹ represents any substituents indicated in [Table L1] to [Table L3](hereinafter, referred to as Compound Class SX₆₀₉).

A compound (L-3) wherein T represents a group represented by T29, R^{3b} represents a bromine atom, R^{3c} represents a hydrogen atom, and R¹ represents any substituents indicated in [Table L1] to [Table L3](hereinafter, referred to as Compound Class SX₆₁₀).

A compound (L-3) wherein T represents a group represented by T29, R^{3b} represents an iodine atom, R^{3c} represents a hydrogen atom, and R¹ represents any substituents indicated in [Table L1] to [Table L3](hereinafter, referred to as Compound Class SX₆₁₁).

A compound (L-3) wherein T represents a group represented by T29, R^{3b} represents a hydrogen atom, R^{3c} represents CF₃, and R¹ represents any substituents indicated in [Table L1] to [Table L3](hereinafter, referred to as Compound Class SX₆₁₂).

A compound (L-3) wherein T represents a group represented by T29, R^{3b} represents a hydrogen atom, R^{3c} represents a chlorine atom, and R¹ represents any substituents indicated in [Table L1] to [Table L3](hereinafter, referred to as Compound Class SX₆₁₃).

A compound (L-3) wherein T represents a group represented by T29, R^{3b} represents a hydrogen atom, R^{3c} represents a bromine atom and R¹ represents any substituents indicated in [Table L1] to [Table L3](hereinafter, referred to as Compound Class SX₆₁₄).

A compound (L-3) wherein T represents a group represented by T29, R^{3b} represents a hydrogen atom, R^{3c} represents an iodine atom and R¹ represents any substituents indicated in [Table L1] to [Table L3](hereinafter, referred to as Compound Class SX₆₁₅).

A compound (L-3) wherein T represents a group represented by T30, R^{3b} represents a hydrogen atom, R^{3c} represents a hydrogen atom, and R¹ represents any substituents indicated in [Table L1] to [Table L3](hereinafter, referred to as Compound Class SX₆₁₆).

A compound (L-3) wherein T represents a group represented by T30, R^{3b} represents CF₃, R^{3c} represents a hydrogen atom, and R¹ represents any substituents indicated in [Table L1] to [Table L3](hereinafter, referred to as Compound Class SX₆₁₇).

A compound (L-3) wherein T represents a group represented by T30, R^{3b} represents a chlorine atom, R^{3c} represents a hydrogen atom, and R¹ represents any substituents indicated in [Table L1] to [Table L3](hereinafter, referred to as Compound Class SX₆₁₈).

A compound (L-3) wherein T represents a group represented by T30, R^{3b} represents a bromine atom, R^{3c} represents a hydrogen atom, and R¹ represents any substituents indicated in [Table L1] to [Table L3](hereinafter, referred to as Compound Class SX₆₁₉).

A compound (L-3) wherein T represents a group represented by T30, R^{3b} represents an iodine atom, R^{3c} represents a hydrogen atom, and R¹ represents any substituents indicated in [Table L1] to [Table L3](hereinafter, referred to as Compound Class SX₆₂₀).

A compound (L-3) wherein T represents a group represented by T30, R^{3b} represents a hydrogen atom, R^{3c} represents CF₃, and R¹ represents any substituents indicated in [Table L1] to [Table L3](hereinafter, referred to as Compound Class SX₆₂₁).

A compound (L-3) wherein T represents a group represented by T30, R^{3b} represents a hydrogen atom, R^{3c} represents a chlorine atom, and R¹ represents any substituents indicated in [Table L1] to [Table L3](hereinafter, referred to as Compound Class SX₆₂₂).

A compound (L-3) wherein T represents a group represented by T30, R^{3b} represents a hydrogen atom, R^{3c} represents a bromine atom and R¹ represents any substituents indicated in [Table L1] to [Table L3](hereinafter, referred to as Compound Class SX₆₂₃).

A compound (L-3) wherein T represents a group represented by T30, R^{3b} represents a hydrogen atom, R^{3c} represents an iodine atom and R¹ represents any substituents indicated in [Table L1] to [Table L3](hereinafter, referred to as Compound Class SX₆₂₄).

A compound (L-3) wherein T represents a group represented by T31, R^{3b} represents a hydrogen atom, R^{3c} represents a hydrogen atom, and R¹ represents any substituents indicated in [Table L1] to [Table L3](hereinafter, referred to as Compound Class SX₆₂₅).

A compound (L-3) wherein T represents a group represented by T31, R^{3b} represents CF₃, R^{3c} represents a hydrogen atom, and R¹ represents any substituents indicated in [Table L1] to [Table L3](hereinafter, referred to as Compound Class SX₆₂₆).

A compound (L-3) wherein T represents a group represented by T31, R^{3b} represents a chlorine atom, R^{3c} represents a hydrogen atom, and R¹ represents any substituents indicated in [Table L1] to [Table L3](hereinafter, referred to as Compound Class SX₆₂₇).

A compound (L-3) wherein T represents a group represented by T31, R^{3b} represents a bromine atom, R^{3c} represents a hydrogen atom, and R¹ represents any substituents indicated in [Table L1] to [Table L3](hereinafter, referred to as Compound Class SX₆₂₈).

A compound (L-3) wherein T represents a group represented by T31, R^{3b} represents an iodine atom, R^{3c} represents a hydrogen atom, and R¹ represents any substituents indicated in [Table L1] to [Table L3](hereinafter, referred to as Compound Class SX₆₂₉).

A compound (L-3) wherein T represents a group represented by T31, R^{3b} represents a hydrogen atom, R^{3c} represents CF₃, and R¹ represents any substituents indicated in [Table L1] to [Table L3](hereinafter, referred to as Compound Class SX₆₃₀).

A compound (L-3) wherein T represents a group represented by T31, R^{3b} represents a hydrogen atom, R^{3c} represents a chlorine atom, and R¹ represents any substituents indicated in [Table L1] to [Table L3](hereinafter, referred to as Compound Class SX₆₃₁).

A compound (L-3) wherein T represents a group represented by T31, R^{3b} represents a hydrogen atom, R^{3c} represents a bromine atom and R¹ represents any substituents indicated in [Table L1] to [Table L3](hereinafter, referred to as Compound Class SX₆₃₂).

A compound (L-3) wherein T represents a group represented by T31, R^{3b} represents a hydrogen atom, R^{3c} represents an iodine atom and R¹ represents any substituents indicated in [Table L1] to [Table L3](hereinafter, referred to as Compound Class SX₆₃₃).

A compound represented by formula (L-4):

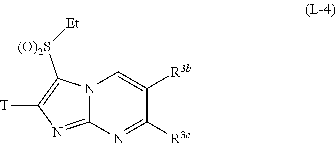

(hereinafter, referred to as compound (L-4)), wherein T represents a group represented by T1, R^{3b} represents a hydrogen atom, R^{3c} represents a hydrogen atom, and R¹ represents any substituents indicated in [Table L1] to [Table L3](hereinafter, referred to as Compound Class SX₆₃₄).

A compound (L-4) wherein T represents a group represented by T1, R^{3b} represents CF₃, R^{3c} represents a hydrogen atom, and R¹ represents any substituents indicated in [Table L1] to [Table L3](hereinafter, referred to as Compound Class SX₆₃₅).

A compound (L-4) wherein T represents a group represented by T1, R^{3b} represents a chlorine atom, R^{3c} represents a hydrogen atom, and R¹ represents any substituents indicated in [Table L1] to [Table L3](hereinafter, referred to as Compound Class SX₆₃₆).

A compound (L-4) wherein T represents a group represented by T1, R^{3b} represents a bromine atom, R^{3c} represents a hydrogen atom, and R¹ represents any substituents indicated in [Table L1] to [Table L3](hereinafter, referred to as Compound Class SX₆₃₇).

A compound (L-4) wherein T represents a group represented by T1, R^{3b} represents an iodine atom, R^{3c} represents a hydrogen atom, and R¹ represents any substituents indicated in [Table L1] to [Table L3](hereinafter, referred to as Compound Class SX₆₃₈).

A compound (L-4) wherein T represents a group represented by T1, R^{3b} represents a hydrogen atom, R^{3c} represents CF₃, and R¹ represents any substituents indicated in [Table L1] to [Table L3](hereinafter, referred to as Compound Class SX₆₃₉).

A compound (L-4) wherein T represents a group represented by T1, R^{3b} represents a hydrogen atom, R^{3c} represents a chlorine atom, and R¹ represents any substituents indicated in [Table L1] to [Table L3](hereinafter, referred to as Compound Class SX₆₄₀).

A compound (L-4) wherein T represents a group represented by T1, R^{3b} represents a hydrogen atom, R^{3c} represents a bromine atom and R¹ represents any substituents indicated in [Table L1] to [Table L3](hereinafter, referred to as Compound Class SX₆₄₁).

A compound (L-4) wherein T represents a group represented by T1, R^{3b} represents a hydrogen atom, R^{3c} represents an iodine atom and R¹ represents any substituents indicated in [Table L1] to [Table L3](hereinafter, referred to as Compound Class SX₆₄₂).

A compound (L-4) wherein T represents a group represented by T2, R^{3b} represents a hydrogen atom, R^{3c} represents a hydrogen atom, and R¹ represents any substituents indicated in [Table L1] to [Table L3](hereinafter, referred to as Compound Class SX₆₄₃).

A compound (L-4) wherein T represents a group represented by T2, R^{3b} represents CF₃, R^{3c} represents a hydrogen atom, and R¹ represents any substituents indicated in [Table L1] to [Table L3](hereinafter, referred to as Compound Class SX₆₄₄).

A compound (L-4) wherein T represents a group represented by T2, R^{3b} represents a chlorine atom, R^{3c} represents a hydrogen atom, and R¹ represents any substituents indicated in [Table L1] to [Table L3](hereinafter, referred to as Compound Class SX₆₄₅).

A compound (L-4) wherein T represents a group represented by T2, R^{3b} represents a bromine atom, R^{3c} represents a hydrogen atom, and R¹ represents any substituents indicated in [Table L1] to [Table L3](hereinafter, referred to as Compound Class SX₆₄₆).

A compound (L-4) wherein T represents a group represented by T2, R^{3b} represents an iodine atom, R^{3c} represents a hydrogen atom, and R¹ represents any substituents indicated in [Table L1] to [Table L3](hereinafter, referred to as Compound Class SX₆₄₇).

A compound (L-4) wherein T represents a group represented by T2, R^{3b} represents a hydrogen atom, R^{3c} represents CF₃, and R¹ represents any substituents indicated in [Table L1] to [Table L3](hereinafter, referred to as Compound Class SX₆₄₈).

A compound (L-4) wherein T represents a group represented by T2, R^{3b} represents a hydrogen atom, R^{3c} represents a chlorine atom, and R¹ represents any substituents indicated in [Table L1] to [Table L3](hereinafter, referred to as Compound Class SX₆₄₉).

A compound (L-4) wherein T represents a group represented by T2, R^{3b} represents a hydrogen atom, R^{3c} represents a bromine atom and R¹ represents any substituents indicated in [Table L1] to [Table L3](hereinafter, referred to as Compound Class SX₆₅₀).

A compound (L-4) wherein T represents a group represented by T2, R^{3b} represents a hydrogen atom, R^{3c} represents an iodine atom and R¹ represents any substituents indicated in [Table L1] to [Table L3](hereinafter, referred to as Compound Class SX₆₅₁).

A compound (L-4) wherein T represents a group represented by T3, R^{3b} represents a hydrogen atom, R^{3c} represents a hydrogen atom, and R¹ represents any substituents indicated in [Table L1] to [Table L3](hereinafter, referred to as Compound Class SX₆₅₂).

A compound (L-4) wherein T represents a group represented by T3, R^{3b} represents CF₃, R^{3c} represents a hydrogen atom, and R¹ represents any substituents indicated in [Table L1] to [Table L3](hereinafter, referred to as Compound Class SX₆₅₃).

A compound (L-4) wherein T represents a group represented by T3, R^{3b} represents a chlorine atom, R^{3c} represents a hydrogen atom, and R¹ represents any substituents indicated in [Table L1] to [Table L3](hereinafter, referred to as Compound Class SX₆₅₄).

A compound (L-4) wherein T represents a group represented by T3, R^{3b} represents a bromine atom, R^{3c} represents a hydrogen atom, and R¹ represents any substituents indicated in [Table L1] to [Table L3](hereinafter, referred to as Compound Class SX₆₅₅).

A compound (L-4) wherein T represents a group represented by T3, R^{3b} represents an iodine atom, R^{3c} represents a hydrogen atom, and R¹ represents any substituents indicated in [Table L1] to [Table L3](hereinafter, referred to as Compound Class SX₆₅₆).

A compound (L-4) wherein T represents a group represented by T3, R^{3b} represents a hydrogen atom, R^{3c} represents CF₃, and R¹ represents any substituents indicated in [Table L1] to [Table L3](hereinafter, referred to as Compound Class SX₆₅₇).

A compound (L-4) wherein T represents a group represented by T3, R^{3b} represents a hydrogen atom, R^{3c} represents a chlorine atom, and R¹ represents any substituents indicated in [Table L1] to [Table L3](hereinafter, referred to as Compound Class SX₆₅₈).

A compound (L-4) wherein T represents a group represented by T3, R^{3b} represents a hydrogen atom, R^{3c} represents a bromine atom and R¹ represents any substituents indicated in [Table L1] to [Table L3](hereinafter, referred to as Compound Class SX₆₅₉).

A compound (L-4) wherein T represents a group represented by T3, R^{3b} represents a hydrogen atom, R^{3c} represents an iodine atom and R¹ represents any substituents indicated in [Table L1] to [Table L3](hereinafter, referred to as Compound Class SX₆₆₀).

A compound (L-4) wherein T represents a group represented by T4, R^{3b} represents a hydrogen atom, R^{3c} represents a hydrogen atom, and R¹ represents any substituents indicated in [Table L1] to [Table L3](hereinafter, referred to as Compound Class SX₆₆₁).

A compound (L-4) wherein T represents a group represented by T4, R^{3b} represents CF₃, R^{3c} represents a hydrogen atom, and R¹ represents any substituents indicated in [Table L1] to [Table L3](hereinafter, referred to as Compound Class SX₆₆₂).

A compound (L-4) wherein T represents a group represented by T4, R^{3b} represents a chlorine atom, R^{3c} represents a hydrogen atom, and R¹ represents any substituents indicated in [Table L1] to [Table L3](hereinafter, referred to as Compound Class SX₆₆₃).

A compound (L-4) wherein T represents a group represented by T4, R^{3b} represents a bromine atom, R^{3c} represents a hydrogen atom, and R¹ represents any substituents indicated in [Table L1] to [Table L3](hereinafter, referred to as Compound Class SX₆₆₄).

A compound (L-4) wherein T represents a group represented by T4, R^{3b} represents an iodine atom, R^{3c} represents a hydrogen atom, and R¹ represents any substituents indicated in [Table L1] to [Table L3](hereinafter, referred to as Compound Class SX₆₆₅).

A compound (L-4) wherein T represents a group represented by T4, R^{3b} represents a hydrogen atom, R^{3c} represents CF₃, and R¹ represents any substituents indicated in [Table L1] to [Table L3](hereinafter, referred to as Compound Class SX₆₆₆).

A compound (L-4) wherein T represents a group represented by T4, R^{3b} represents a hydrogen atom, R^{3c} represents a chlorine atom, and R¹ represents any substituents indicated in [Table L1] to [Table L3](hereinafter, referred to as Compound Class SX₆₆₇).

A compound (L-4) wherein T represents a group represented by T4, R^{3b} represents a hydrogen atom, R^{3c} represents a bromine atom and R¹ represents any substituents indicated in [Table L1] to [Table L3](hereinafter, referred to as Compound Class SX₆₆₈).

A compound (L-4) wherein T represents a group represented by T4, R^{3b} represents a hydrogen atom, R^{3c} represents an iodine atom and R¹ represents any substituents indicated in [Table L1] to [Table L3](hereinafter, referred to as Compound Class SX₆₆₉).

A compound (L-4) wherein T represents a group represented by T5, R^{3b} represents a hydrogen atom, R^{3c} represents a hydrogen atom, and R¹ represents any substituents indicated in [Table L1] to [Table L3](hereinafter, referred to as Compound Class SX₆₇₀).

A compound (L-4) wherein T represents a group represented by T5, R^{3b} represents CF₃, R^{3c} represents a hydrogen atom, and R¹ represents any substituents indicated in [Table L1] to [Table L3](hereinafter, referred to as Compound Class SX₆₇₁).

A compound (L-4) wherein T represents a group represented by T5, R^{3b} represents a chlorine atom, R^{3c} represents a hydrogen atom, and R¹ represents any substituents indicated in [Table L1] to [Table L3](hereinafter, referred to as Compound Class SX₆₇₂).

A compound (L-4) wherein T represents a group represented by T5, R^{3b} represents a bromine atom, R^{3c} represents a hydrogen atom, and R¹ represents any substituents indicated in [Table L1] to [Table L3](hereinafter, referred to as Compound Class SX₆₇₃).

A compound (L-4) wherein T represents a group represented by T5, R^{3b} represents an iodine atom, R^{3c} represents a hydrogen atom, and R¹ represents any substituents indicated in [Table L1] to [Table L3](hereinafter, referred to as Compound Class SX₆₇₄).

A compound (L-4) wherein T represents a group represented by T5, R^{3b} represents a hydrogen atom, R^{3c} represents CF₃, and R¹ represents any substituents indicated in [Table L1] to [Table L3](hereinafter, referred to as Compound Class SX₆₇₅).

A compound (L-4) wherein T represents a group represented by T5, R^{3b} represents a hydrogen atom, R^{3c} represents a chlorine atom, and R¹ represents any substituents indicated in [Table L1] to [Table L3](hereinafter, referred to as Compound Class SX₆₇₆).

A compound (L-4) wherein T represents a group represented by T5, R^{3b} represents a hydrogen atom, R^{3c} represents a bromine atom and R¹ represents any substituents indicated in [Table L1] to [Table L3](hereinafter, referred to as Compound Class SX₆₇₇).

A compound (L-4) wherein T represents a group represented by T5, R^{3b} represents a hydrogen atom, R^{3c} represents an iodine atom and R¹ represents any substituents indicated in [Table L1] to [Table L3](hereinafter, referred to as Compound Class SX₆₇₈).

A compound (L-4) wherein T represents a group represented by T6, R^{3b} represents a hydrogen atom, R^{3c} represents a hydrogen atom, and R¹ represents any substituents indicated in [Table L1] to [Table L3](hereinafter, referred to as Compound Class SX₆₇₉).

A compound (L-4) wherein T represents a group represented by T6, R^{3b} represents CF₃, R^{3c} represents a hydrogen atom, and R¹ represents any substituents indicated in [Table L1] to [Table L3](hereinafter, referred to as Compound Class SX₆₈₀).

A compound (L-4) wherein T represents a group represented by T6, R^{3b} represents a chlorine atom, R^{3c} represents a hydrogen atom, and R¹ represents any substituents indicated in [Table L1] to [Table L3](hereinafter, referred to as Compound Class SX₆₈₁).

A compound (L-4) wherein T represents a group represented by T6, R^{3b} represents a bromine atom, R^{3c} represents a hydrogen atom, and R¹ represents any substituents indicated in [Table L1] to [Table L3](hereinafter, referred to as Compound Class SX₆₈₂).

A compound (L-4) wherein T represents a group represented by T6, R^{3b} represents an iodine atom, R^{3c} represents a hydrogen atom, and R¹ represents any substituents indicated in [Table L1] to [Table L3](hereinafter, referred to as Compound Class SX₆₈₃).

A compound (L-4) wherein T represents a group represented by T6, R^{3b} represents a hydrogen atom, R^{3c} represents CF₃, and R¹ represents any substituents indicated in [Table L1] to [Table L3](hereinafter, referred to as Compound Class SX₆₈₄).

A compound (L-4) wherein T represents a group represented by T6, R^{3b} represents a hydrogen atom, R^{3c} represents a chlorine atom, and R¹ represents any substituents indicated in [Table L1] to [Table L3](hereinafter, referred to as Compound Class SX₆₈₅).

A compound (L-4) wherein T represents a group represented by T6, R^{3b} represents a hydrogen atom, R^{3c} represents a bromine atom and R¹ represents any substituents indicated in [Table L1] to [Table L3](hereinafter, referred to as Compound Class SX₆₈₆).

A compound (L-4) wherein T represents a group represented by T6, R^{3b} represents a hydrogen atom, R^{3c} represents an iodine atom and R¹ represents any substituents indicated in [Table L1] to [Table L3](hereinafter, referred to as Compound Class SX₆₈₇).

A compound (L-4) wherein T represents a group represented by T7, R^{3b} represents a hydrogen atom, R^{3c} represents a hydrogen atom, and R¹ represents any substituents indicated in [Table L1] to [Table L3](hereinafter, referred to as Compound Class SX₆₈₈).

A compound (L-4) wherein T represents a group represented by T7, R^{3b} represents CF₃, R^{3c} represents a hydrogen atom, and R¹ represents any substituents indicated in [Table L1] to [Table L3](hereinafter, referred to as Compound Class SX₆₈₉).

A compound (L-4) wherein T represents a group represented by T7, R^{3b} represents a chlorine atom, R^{3c} represents a hydrogen atom, and R¹ represents any substituents indicated in [Table L1] to [Table L3](hereinafter, referred to as Compound Class SX₆₉₀).

A compound (L-4) wherein T represents a group represented by T7, R^{3b} represents a bromine atom, R^{3c} represents a hydrogen atom, and R¹ represents any substituents indicated in [Table L1] to [Table L3](hereinafter, referred to as Compound Class SX₆₉₁).

A compound (L-4) wherein T represents a group represented by T7, R^{3b} represents an iodine atom, R^{3c} represents a hydrogen atom, and R¹ represents any substituents indicated in [Table L1] to [Table L3](hereinafter, referred to as Compound Class SX₆₉₂).

A compound (L-4) wherein T represents a group represented by T7, R^{3b} represents a hydrogen atom, R^{3c} represents CF₃, and R¹ represents any substituents indicated in [Table L1] to [Table L3](hereinafter, referred to as Compound Class SX₆₉₂).

A compound (L-4) wherein T represents a group represented by T7, R^{3b} represents a hydrogen atom, R^{3c} represents a chlorine atom, and R¹ represents any substituents indicated in [Table L1] to [Table L3](hereinafter, referred to as Compound Class SX₆₉₃).

A compound (L-4) wherein T represents a group represented by T7, R^{3b} represents a hydrogen atom, R^{3c} represents a bromine atom and R¹ represents any substituents indicated in [Table L1] to [Table L3](hereinafter, referred to as Compound Class SX₆₉₄).

A compound (L-4) wherein T represents a group represented by T7, R^{3b} represents a hydrogen atom, R^{3c} represents an iodine atom and R¹ represents any substituents indicated in [Table L1] to [Table L3](hereinafter, referred to as Compound Class SX₆₉₅).

A compound (L-4) wherein T represents a group represented by T8, R^{3b} represents a hydrogen atom, R^{3c} represents a hydrogen atom, and R¹ represents any substituents indicated in [Table L1] to [Table L3](hereinafter, referred to as Compound Class SX₆₉₆).

A compound (L-4) wherein T represents a group represented by T8, R^{3b} represents CF₃, R^{3c} represents a hydrogen atom, and R¹ represents any substituents indicated in [Table L1] to [Table L3](hereinafter, referred to as Compound Class SX₆₉₇).

A compound (L-4) wherein T represents a group represented by T8, R^{3b} represents a chlorine atom, R^{3c} represents a hydrogen atom, and R¹ represents any substituents indicated in [Table L1] to [Table L3](hereinafter, referred to as Compound Class SX₆₉₉).

A compound (L-4) wherein T represents a group represented by T8, R^{3b} represents a bromine atom, R^{3c} represents a hydrogen atom, and R¹ represents any substituents indicated in [Table L1] to [Table L3](hereinafter, referred to as Compound Class SX₇₀₀).

A compound (L-4) wherein T represents a group represented by T8, R^{3b} represents an iodine atom, R^{3c} represents a hydrogen atom, and R¹ represents any substituents indicated in [Table L1] to [Table L3](hereinafter, referred to as Compound Class SX₇₀₁).

A compound (L-4) wherein T represents a group represented by T8, R^{3b} represents a hydrogen atom, R^{3c} represents CF₃, and R¹ represents any substituents indicated in [Table L1] to [Table L3](hereinafter, referred to as Compound Class SX₇₀₂).

A compound (L-4) wherein T represents a group represented by T8, R^{3b} represents a hydrogen atom, R^{3c} represents a chlorine atom, and R¹ represents any substituents indicated in [Table L1] to [Table L3](hereinafter, referred to as Compound Class SX₇₀₃).

A compound (L-4) wherein T represents a group represented by T8, R^{3b} represents a hydrogen atom, R^{3c} represents a bromine atom and R¹ represents any substituents indicated in [Table L1] to [Table L3](hereinafter, referred to as Compound Class SX₇₀₄).

A compound (L-4) wherein T represents a group represented by T8, R^{3b} represents a hydrogen atom, R^{3c} represents an iodine atom and R¹ represents any substituents indicated in [Table L1] to [Table L3](hereinafter, referred to as Compound Class SX₇₀₅).

A compound (L-4) wherein T represents a group represented by T9, R^{3b} represents a hydrogen atom, R^{3c} represents a hydrogen atom, and R¹ represents any substituents indicated in [Table L1] to [Table L3](hereinafter, referred to as Compound Class SX₇₀₆).

A compound (L-4) wherein T represents a group represented by T9, R^{3b} represents CF₃, R^{3c} represents a hydrogen atom, and R¹ represents any substituents indicated in [Table L1] to [Table L3](hereinafter, referred to as Compound Class SX₇₀₇).

A compound (L-4) wherein T represents a group represented by T9, R^{3b} represents a chlorine atom, R^{3c} represents a hydrogen atom, and R¹ represents any substituents indicated in [Table L1] to [Table L3](hereinafter, referred to as Compound Class SX₇₀₈).

A compound (L-4) wherein T represents a group represented by T9, R^{3b} represents a bromine atom, R^{3c} represents a hydrogen atom, and R¹ represents any substituents indicated in [Table L1] to [Table L3](hereinafter, referred to as Compound Class SX₇₀₉).

A compound (L-4) wherein T represents a group represented by T7, R^{3b} represents an iodine atom, R^{3c} represents a hydrogen atom, and R¹ represents any substituents indicated in [Table L1] to [Table L3](hereinafter, referred to as Compound Class SX₇₁₀).

A compound (L-4) wherein T represents a group represented by T9, R^{3b} represents a hydrogen atom, R^{3c} represents CF₃, and R¹ represents any substituents indicated in [Table L1] to [Table L3](hereinafter, referred to as Compound Class SX₇₁₁).

A compound (L-4) wherein T represents a group represented by T9, R^{3b} represents a hydrogen atom, R^{3c} represents a chlorine atom, and R¹ represents any substituents indicated in [Table L1] to [Table L3](hereinafter, referred to as Compound Class SX₇₁₂).

A compound (L-4) wherein T represents a group represented by T9, R^{3b} represents a hydrogen atom, R^{3c} represents a bromine atom and R¹ represents any substituents indicated in [Table L1] to [Table L3](hereinafter, referred to as Compound Class SX₇₁₃).

A compound (L-4) wherein T represents a group represented by T9, R^{3b} represents a hydrogen atom, R^{3c} represents an iodine atom and R¹ represents any substituents indicated in [Table L1] to [Table L3](hereinafter, referred to as Compound Class SX₇₁₄).

A compound (L-4) wherein T represents a group represented by T10, R^{3b} represents a hydrogen atom, R^{3c} represents a hydrogen atom, and R¹ represents any substituents indicated in [Table L1] to [Table L3](hereinafter, referred to as Compound Class SX₇₁₅).

A compound (L-4) wherein T represents a group represented by T10, R^{3b} represents CF₃, R^{3c} represents a hydrogen atom, and R¹ represents any substituents indicated in [Table L1] to [Table L3](hereinafter, referred to as Compound Class SX₇₁₆).

A compound (L-4) wherein T represents a group represented by T10, R^{3b} represents a chlorine atom, R^{3c} represents a hydrogen atom, and R¹ represents any substituents indicated in [Table L1] to [Table L3](hereinafter, referred to as Compound Class SX₇₁₇).

A compound (L-4) wherein T represents a group represented by T10, R^{3b} represents a bromine atom, R^{3c} represents a hydrogen atom, and R¹ represents any substituents indicated in [Table L1] to [Table L3](hereinafter, referred to as Compound Class SX₇₁₈).

A compound (L-4) wherein T represents a group represented by T10, R^{3b} represents an iodine atom, R^{3c} represents a hydrogen atom, and R¹ represents any substituents indicated in [Table L1] to [Table L3](hereinafter, referred to as Compound Class SX₇₁₉).

A compound (L-4) wherein T represents a group represented by T10, R^{3b} represents a hydrogen atom, R^{3c} represents CF₃, and R¹ represents any substituents indicated in [Table L1] to [Table L3](hereinafter, referred to as Compound Class SX₇₂₀).

A compound (L-4) wherein T represents a group represented by T10, R^{3b} represents a hydrogen atom, R^{3c} represents a chlorine atom, and R¹ represents any substituents indicated in [Table L1] to [Table L3](hereinafter, referred to as Compound Class SX₇₂₁).

A compound (L-4) wherein T represents a group represented by T10, R^{3b} represents a hydrogen atom, R^{3c} represents a bromine atom and R¹ represents any substituents indicated in [Table L1] to [Table L3](hereinafter, referred to as Compound Class SX₇₂₂).

A compound (L-4) wherein T represents a group represented by T10, R^{3b} represents a hydrogen atom, R^{3c} represents an iodine atom and R¹ represents any substituents indicated in [Table L1] to [Table L3](hereinafter, referred to as Compound Class SX₇₂₃).

A compound (L-4) wherein T represents a group represented by T1l, R^{3b} represents a hydrogen atom, R^{3c} represents a hydrogen atom, and R¹ represents any substituents indicated in [Table L1] to [Table L3](hereinafter, referred to as Compound Class SX₇₂₄).

A compound (L-4) wherein T represents a group represented by T1l, R^{3b} represents CF₃, R^{3c} represents a hydrogen atom, and R¹ represents any substituents indicated in [Table L1] to [Table L3](hereinafter, referred to as Compound Class SX₇₂₅).

A compound (L-4) wherein T represents a group represented by T1l, R^{3b} represents a chlorine atom, R^{3c} represents a hydrogen atom, and R¹ represents any substituents indicated in [Table L1] to [Table L3](hereinafter, referred to as Compound Class SX₇₂₆).

A compound (L-4) wherein T represents a group represented by T1l, R^{3b} represents a bromine atom, R^{3c} represents a hydrogen atom, and R¹ represents any substituents indicated in [Table L1] to [Table L3](hereinafter, referred to as Compound Class SX₇₂₇).

A compound (L-4) wherein T represents a group represented by T1l, R^{3b} represents an iodine atom, R^{3c} represents a hydrogen atom, and R¹ represents any substituents indicated in [Table L1] to [Table L3](hereinafter, referred to as Compound Class SX₇₂₈).

A compound (L-4) wherein T represents a group represented by T1l, R^{3b} represents a hydrogen atom, R^{3c} represents CF₃, and R¹ represents any substituents indicated in [Table L1] to [Table L3](hereinafter, referred to as Compound Class SX₇₂₉).

A compound (L-4) wherein T represents a group represented by T1l, R^{3b} represents a hydrogen atom, R^{3c} represents a chlorine atom, and R¹ represents any substituents indicated in [Table L1] to [Table L3](hereinafter, referred to as Compound Class SX₇₃₀).

A compound (L-4) wherein T represents a group represented by T1l, R^{3b} represents a hydrogen atom, R^{3c} represents a bromine atom and R¹ represents any substituents indicated in [Table L1] to [Table L3](hereinafter, referred to as Compound Class SX₇₃₁).

A compound (L-4) wherein T represents a group represented by T1l, R^{3b} represents a hydrogen atom, R^{3c} represents an iodine atom and R¹ represents any substituents indicated in [Table L1] to [Table L3](hereinafter, referred to as Compound Class SX₇₃₂).

A compound (L-4) wherein T represents a group represented by T12, R^{3b} represents a hydrogen atom, R^{3c} represents a hydrogen atom, and R¹ represents any substituents indicated in [Table L1] to [Table L3](hereinafter, referred to as Compound Class SX₇₃₃).

A compound (L-4) wherein T represents a group represented by T12, R^{3b} represents CF₃, R^{3c} represents a hydrogen atom, and R¹ represents any substituents indicated in [Table L1] to [Table L3](hereinafter, referred to as Compound Class SX₇₃₄).

A compound (L-4) wherein T represents a group represented by T12, R^{3b} represents a chlorine atom, R^{3c} represents a hydrogen atom, and R¹ represents any substituents indicated in [Table L1] to [Table L3](hereinafter, referred to as Compound Class SX₇₃₅).

A compound (L-4) wherein T represents a group represented by T12, R^{3b} represents a bromine atom, R^{3c} represents a hydrogen atom, and R¹ represents any substituents indicated in [Table L1] to [Table L3](hereinafter, referred to as Compound Class SX₇₃₆).

A compound (L-4) wherein T represents a group represented by T12, R^{3b} represents an iodine atom, R^{3c} represents a hydrogen atom, and R¹ represents any substituents indicated in [Table L1] to [Table L3](hereinafter, referred to as Compound Class SX₇₃₇).

A compound (L-4) wherein T represents a group represented by T12, R^{3b} represents a hydrogen atom, R^{3c} represents CF₃, and R¹ represents any substituents indicated in [Table L1] to [Table L3](hereinafter, referred to as Compound Class SX₇₃₈).

A compound (L-4) wherein T represents a group represented by T12, R^{3b} represents a hydrogen atom, R^{3c} represents a chlorine atom, and R¹ represents any substituents indicated in [Table L1] to [Table L3](hereinafter, referred to as Compound Class SX₇₃₉).

A compound (L-4) wherein T represents a group represented by T12, R^{3b} represents a hydrogen atom, R^{3c} represents a bromine atom and R¹ represents any substituents indicated in [Table L1] to [Table L3](hereinafter, referred to as Compound Class SX₇₄₀).

A compound (L-4) wherein T represents a group represented by T12, R^{3b} represents a hydrogen atom, R^{3c} represents an iodine atom and R¹ represents any substituents indicated in [Table L1] to [Table L3](hereinafter, referred to as Compound Class SX₇₄₁).

A compound (L-4) wherein T represents a group represented by T13, R^{3b} represents a hydrogen atom, R^{3c} represents a hydrogen atom, and R¹ represents any substituents indicated in [Table L1] to [Table L3](hereinafter, referred to as Compound Class SX₇₄₂).

A compound (L-4) wherein T represents a group represented by T13, R^{3b} represents CF₃, R^{3c} represents a hydrogen atom, and R¹ represents any substituents indicated in [Table L1] to [Table L3](hereinafter, referred to as Compound Class SX₇₄₃).

A compound (L-4) wherein T represents a group represented by T13, R^{3b} represents a chlorine atom, R^{3c} represents a hydrogen atom, and R¹ represents any substituents indicated in [Table L1] to [Table L3](hereinafter, referred to as Compound Class SX₇₄₄).

A compound (L-4) wherein T represents a group represented by T13, R^{3b} represents a bromine atom, R^{3c} represents a hydrogen atom, and R¹ represents any substituents indicated in [Table L1] to [Table L3](hereinafter, referred to as Compound Class SX₇₄₅).

A compound (L-4) wherein T represents a group represented by T13, R^{3b} represents an iodine atom, R^{3c} represents a hydrogen atom, and R¹ represents any substituents indicated in [Table L1] to [Table L3](hereinafter, referred to as Compound Class SX₇₄₆).

A compound (L-4) wherein T represents a group represented by T13, R^{3b} represents a hydrogen atom, R^{3c} represents CF₃, and R¹ represents any substituents indicated in [Table L1] to [Table L3](hereinafter, referred to as Compound Class SX₇₄₇).

A compound (L-4) wherein T represents a group represented by T13, R^{3b} represents a hydrogen atom, R^{3c} represents a chlorine atom, and R¹ represents any substituents indicated in [Table L1] to [Table L3](hereinafter, referred to as Compound Class SX₇₄₈).

A compound (L-4) wherein T represents a group represented by T13, R^{3b} represents a hydrogen atom, R^{3c} represents a bromine atom and R¹ represents any substituents indicated in [Table L1] to [Table L3](hereinafter, referred to as Compound Class SX₇₄₉).

A compound (L-4) wherein T represents a group represented by T13, R^{3b} represents a hydrogen atom, R^{3c} represents an iodine atom and R¹ represents any substituents indicated in [Table L1] to [Table L3](hereinafter, referred to as Compound Class SX₇₅₀).

A compound (L-4) wherein T represents a group represented by T14, R^{3b} represents a hydrogen atom, R^{3c} represents a hydrogen atom, and R¹ represents any substituents indicated in [Table L1] to [Table L3](hereinafter, referred to as Compound Class SX₇₅₁).

A compound (L-4) wherein T represents a group represented by T14, R^{3b} represents CF₃, R^{3c} represents a hydrogen atom, and R¹ represents any substituents indicated in [Table L1] to [Table L3](hereinafter, referred to as Compound Class SX₇₅₂).

A compound (L-4) wherein T represents a group represented by T14, R^{3b} represents a chlorine atom, R^{3c} represents a hydrogen atom, and R¹ represents any substituents indicated in [Table L1] to [Table L3](hereinafter, referred to as Compound Class SX₇₅₃).

A compound (L-4) wherein T represents a group represented by T14, R^{3b} represents a bromine atom, R^{3c} represents a hydrogen atom, and R¹ represents any substituents indicated in [Table L1] to [Table L3](hereinafter, referred to as Compound Class SX₇₅₄).

A compound (L-4) wherein T represents a group represented by T14, R^{3b} represents an iodine atom, R^{3c} represents a hydrogen atom, and R¹ represents any substituents indicated in [Table L1] to [Table L3](hereinafter, referred to as Compound Class SX₇₅₅).

A compound (L-4) wherein T represents a group represented by T14, R^{3b} represents a hydrogen atom, R^{3c} represents CF₃, and R¹ represents any substituents indicated in [Table L1] to [Table L3](hereinafter, referred to as Compound Class SX₇₅₆).

A compound (L-4) wherein T represents a group represented by T14, R^{3b} represents a hydrogen atom, R^{3c} represents a chlorine atom, and R¹ represents any substituents indicated in [Table L1] to [Table L3](hereinafter, referred to as Compound Class SX₇₅₇).

A compound (L-4) wherein T represents a group represented by T14, R^{3b} represents a hydrogen atom, R^{3c} represents a bromine atom and R¹ represents any substituents indicated in [Table L1] to [Table L3](hereinafter, referred to as Compound Class SX₇₅₈).

A compound (L-4) wherein T represents a group represented by T14, R^{3b} represents a hydrogen atom, R^{3c} represents an iodine atom and R¹ represents any substituents indicated in [Table L1] to [Table L3](hereinafter, referred to as Compound Class SX₇₅₉).

A compound (L-4) wherein T represents a group represented by T15, R^{3b} represents a hydrogen atom, R^{3c} represents a hydrogen atom, and R¹ represents any substituents indicated in [Table L1] to [Table L3](hereinafter, referred to as Compound Class SX₇₆₀).

A compound (L-4) wherein T represents a group represented by T15, R^{3b} represents CF₃, R^{3c} represents a hydrogen atom, and R¹ represents any substituents indicated in [Table L1] to [Table L3](hereinafter, referred to as Compound Class SX₇₆₁).

A compound (L-4) wherein T represents a group represented by T15, R^{3b} represents a chlorine atom, R^{3c} represents a hydrogen atom, and R¹ represents any substituents indicated in [Table L1] to [Table L3](hereinafter, referred to as Compound Class SX₇₆₂).

A compound (L-4) wherein T represents a group represented by T15, R^{3b} represents a bromine atom, R^{3c} represents a hydrogen atom, and R¹ represents any substituents indicated in [Table L1] to [Table L3](hereinafter, referred to as Compound Class SX₇₆₃).

A compound (L-4) wherein T represents a group represented by T15, R^{3b} represents an iodine atom, R^{3c} represents a hydrogen atom, and R¹ represents any substituents indicated in [Table L1] to [Table L3](hereinafter, referred to as Compound Class SX₇₆₄).

A compound (L-4) wherein T represents a group represented by T15, R^{3b} represents a hydrogen atom, R^{3c} represents CF₃, and R¹ represents any substituents indicated in [Table L1] to [Table L3](hereinafter, referred to as Compound Class SX₇₆₅).

A compound (L-4) wherein T represents a group represented by T15, R^{3b} represents a hydrogen atom, R^{3c} represents a chlorine atom, and R¹ represents any substituents indicated in [Table L1] to [Table L3](hereinafter, referred to as Compound Class SX₇₆₆).

A compound (L-4) wherein T represents a group represented by T15, R^{3b} represents a hydrogen atom, R^{3c} represents a bromine atom and R¹ represents any substituents indicated in [Table L1] to [Table L3](hereinafter, referred to as Compound Class SX₇₆₇).

A compound (L-4) wherein T represents a group represented by T15, R^{3b} represents a hydrogen atom, R^{3c} represents an iodine atom and R¹ represents any substituents indicated in [Table L1] to [Table L3](hereinafter, referred to as Compound Class SX₇₆₈).

A compound (L-4) wherein T represents a group represented by T16, R^{3b} represents a hydrogen atom, R^{3c} represents a hydrogen atom, and R¹ represents any substituents indicated in [Table L1] to [Table L3](hereinafter, referred to as Compound Class SX₇₆₉).

A compound (L-4) wherein T represents a group represented by T16, R^{3b} represents CF₃, R^{3c} represents a hydrogen atom, and R¹ represents any substituents indicated in [Table L1] to [Table L3](hereinafter, referred to as Compound Class SX₇₇₀).

A compound (L-4) wherein T represents a group represented by T16, R^{3b} represents a chlorine atom, R^{3c} represents a hydrogen atom, and R¹ represents any substituents indicated in [Table L1] to [Table L3](hereinafter, referred to as Compound Class SX₇₇₁).

A compound (L-4) wherein T represents a group represented by T16, R^{3b} represents a bromine atom, R^{3c} represents a hydrogen atom, and R¹ represents any substituents indicated in [Table L1] to [Table L3](hereinafter, referred to as Compound Class SX₇₇₂).

A compound (L-4) wherein T represents a group represented by T16, R^{3b} represents an iodine atom, R^{3c} represents a hydrogen atom, and R¹ represents any substituents indicated in [Table L1] to [Table L3](hereinafter, referred to as Compound Class SX₇₇₃).

A compound (L-4) wherein T represents a group represented by T16, R^{3b} represents a hydrogen atom, R^{3c} represents CF₃, and R¹ represents any substituents indicated in [Table L1] to [Table L3](hereinafter, referred to as Compound Class SX₇₇₄).

A compound (L-4) wherein T represents a group represented by T16, R^{3b} represents a hydrogen atom, R^{3c} represents a chlorine atom, and R¹ represents any substituents indicated in [Table L1] to [Table L3](hereinafter, referred to as Compound Class SX₇₇₅).

A compound (L-4) wherein T represents a group represented by T16, R^{3b} represents a hydrogen atom, R^{3c} represents a bromine atom and R¹ represents any substituents indicated in [Table L1] to [Table L3](hereinafter, referred to as Compound Class SX₇₇₆).

A compound (L-4) wherein T represents a group represented by T16, R^{3b} represents a hydrogen atom, R^{3c} represents an iodine atom and R¹ represents any substituents indicated in [Table L1] to [Table L3](hereinafter, referred to as Compound Class SX₇₇₇).

A compound (L-4) wherein T represents a group represented by T17, R^{3b} represents a hydrogen atom, R^{3c} represents a hydrogen atom, and R¹ represents any substituents indicated in [Table L1] to [Table L3](hereinafter, referred to as Compound Class SX₇₇₈).

A compound (L-4) wherein T represents a group represented by T17, R^{3b} represents CF₃, R^{3c} represents a hydrogen atom, and R¹ represents any substituents indicated in [Table L1] to [Table L3](hereinafter, referred to as Compound Class SX₇₇₉).

A compound (L-4) wherein T represents a group represented by T17, R^{3b} represents a chlorine atom, R^{3c} represents a hydrogen atom, and R¹ represents any substituents indicated in [Table L1] to [Table L3](hereinafter, referred to as Compound Class SX₇₈₀).

A compound (L-4) wherein T represents a group represented by T17, R^{3b} represents a bromine atom, R^{3c} represents a hydrogen atom, and R¹ represents any substituents indicated in [Table L1] to [Table L3](hereinafter, referred to as Compound Class SX₇₈₁).

A compound (L-4) wherein T represents a group represented by T17, R^{3b} represents an iodine atom, R^{3c} represents a hydrogen atom, and R¹ represents any substituents indicated in [Table L1] to [Table L3](hereinafter, referred to as Compound Class SX₇₈₂).

A compound (L-4) wherein T represents a group represented by T17, R^{3b} represents a hydrogen atom, R^{3c} represents CF₃, and R¹ represents any substituents indicated in [Table L1] to [Table L3](hereinafter, referred to as Compound Class SX₇₈₃).

A compound (L-4) wherein T represents a group represented by T17, R^{3b} represents a hydrogen atom, R^{3c} represents a chlorine atom, and R¹ represents any substituents indicated in [Table L1] to [Table L3](hereinafter, referred to as Compound Class SX₇₈₄).

A compound (L-4) wherein T represents a group represented by T17, R^{3b} represents a hydrogen atom, R^{3c} represents a bromine atom and R¹ represents any substituents indicated in [Table L1] to [Table L3](hereinafter, referred to as Compound Class SX₇₈₅).

A compound (L-4) wherein T represents a group represented by T17, R^{3b} represents a hydrogen atom, R^{3c} represents an iodine atom and R¹ represents any substituents indicated in [Table L1] to [Table L3](hereinafter, referred to as Compound Class SX₇₈₆).

A compound (L-4) wherein T represents a group represented by T18, R^{3b} represents a hydrogen atom, R^{3c} represents a hydrogen atom, and R¹ represents any substituents indicated in [Table L1] to [Table L3](hereinafter, referred to as Compound Class SX₇₈₇).

A compound (L-4) wherein T represents a group represented by T18, R^{3b} represents CF₃, R^{3c} represents a hydrogen atom, and R¹ represents any substituents indicated in [Table L1] to [Table L3](hereinafter, referred to as Compound Class SX₇₈₈).

A compound (L-4) wherein T represents a group represented by T18, R^{3b} represents a chlorine atom, R^{3c} represents a hydrogen atom, and R¹ represents any substituents indicated in [Table L1] to [Table L3](hereinafter, referred to as Compound Class SX₇₈₉).

A compound (L-4) wherein T represents a group represented by T18, R^{3b} represents a bromine atom, R^{3c} represents a hydrogen atom, and R¹ represents any substituents indicated in [Table L1] to [Table L3](hereinafter, referred to as Compound Class SX₇₉₀).

A compound (L-4) wherein T represents a group represented by T18, R^{3b} represents an iodine atom, R^{3c} represents a hydrogen atom, and R¹ represents any substituents indicated in [Table L1] to [Table L3](hereinafter, referred to as Compound Class SX₇₉₁).

A compound (L-4) wherein T represents a group represented by T18, R^{3b} represents a hydrogen atom, R^{3c} represents CF₃, and R¹ represents any substituents indicated in [Table L1] to [Table L3](hereinafter, referred to as Compound Class SX₇₉₂).

A compound (L-4) wherein T represents a group represented by T18, R^{3b} represents a hydrogen atom, R^{3c} represents a chlorine atom, and R¹ represents any substituents indicated in [Table L1] to [Table L3](hereinafter, referred to as Compound Class SX₇₉₃).

A compound (L-4) wherein T represents a group represented by T18, R^{3b} represents a hydrogen atom, R^{3c} represents a bromine atom and R¹ represents any substituents indicated in [Table L1] to [Table L3](hereinafter, referred to as Compound Class SX₇₉₄).

A compound (L-4) wherein T represents a group represented by T18, R^{3b} represents a hydrogen atom, R^{3c} represents an iodine atom and R¹ represents any substituents indicated in [Table L1] to [Table L3](hereinafter, referred to as Compound Class SX₇₉₅).

A compound (L-4) wherein T represents a group represented by T19, R^{3b} represents a hydrogen atom, R^{3c} represents a hydrogen atom, and R¹ represents any substituents indicated in [Table L1] to [Table L3](hereinafter, referred to as Compound Class SX₇₉₆).

A compound (L-4) wherein T represents a group represented by T19, R^{3b} represents CF₃, R^{3c} represents a hydrogen atom, and R¹ represents any substituents indicated in [Table L1] to [Table L3](hereinafter, referred to as Compound Class SX₇₉₇).

A compound (L-4) wherein T represents a group represented by T19, R^{3b} represents a chlorine atom, R^{3c} represents a hydrogen atom, and R¹ represents any substituents indicated in [Table L1] to [Table L3](hereinafter, referred to as Compound Class SX₇₉₈).

A compound (L-4) wherein T represents a group represented by T19, R^{3b} represents a bromine atom, R^{3c} represents a hydrogen atom, and R¹ represents any substituents indicated in [Table L1] to [Table L3](hereinafter, referred to as Compound Class SX₇₉₉).

A compound (L-4) wherein T represents a group represented by T19, R^{3b} represents an iodine atom, R^{3c} represents a hydrogen atom, and R¹ represents any substituents indicated in [Table L1] to [Table L3](hereinafter, referred to as Compound Class SX₈₀₀).

A compound (L-4) wherein T represents a group represented by T19, R^{3b} represents a hydrogen atom, R^{3c} represents CF₃, and R¹ represents any substituents indicated in [Table L1] to [Table L3](hereinafter, referred to as Compound Class SX₈₀₁).

A compound (L-4) wherein T represents a group represented by T19, R^{3b} represents a hydrogen atom, R^{3c} represents a chlorine atom, and R¹ represents any substituents indicated in [Table L1] to [Table L3](hereinafter, referred to as Compound Class SX₈₀₂).

A compound (L-4) wherein T represents a group represented by T19, R^{3b} represents a hydrogen atom, R^{3c} represents a bromine atom and R¹ represents any substituents indicated in [Table L1] to [Table L3](hereinafter, referred to as Compound Class SX₈₀₃).

A compound (L-4) wherein T represents a group represented by T19, R^{3b} represents a hydrogen atom, R^{3c} represents an iodine atom and R¹ represents any substituents indicated in [Table L1] to [Table L3](hereinafter, referred to as Compound Class SX₈₀₄).

A compound (L-4) wherein T represents a group represented by T20, R^{3b} represents a hydrogen atom, R^{3c} represents a hydrogen atom, and R¹ represents any substituents indicated in [Table L1] to [Table L3](hereinafter, referred to as Compound Class SX₈₀₅).

A compound (L-4) wherein T represents a group represented by T20, R^{3b} represents CF₃, R^{3c} represents a hydrogen atom, and R¹ represents any substituents indicated in [Table L1] to [Table L3](hereinafter, referred to as Compound Class SX₈₀₆).

A compound (L-4) wherein T represents a group represented by T20, R^{3b} represents a chlorine atom, R^{3c} represents a hydrogen atom, and R¹ represents any substituents indicated in [Table L1] to [Table L3](hereinafter, referred to as Compound Class SX₈₀₇).

A compound (L-4) wherein T represents a group represented by T20, R^{3b} represents a bromine atom, R^{3c} represents a hydrogen atom, and R¹ represents any substituents indicated in [Table L1] to [Table L3](hereinafter, referred to as Compound Class SX₈₀₈).

A compound (L-4) wherein T represents a group represented by T20, R^{3b} represents an iodine atom, R^{3c} represents a hydrogen atom, and R¹ represents any substituents indicated in [Table L1] to [Table L3](hereinafter, referred to as Compound Class SX₈₀₉).

A compound (L-4) wherein T represents a group represented by T20, R^{3b} represents a hydrogen atom, R^{3c} represents CF₃, and R¹ represents any substituents indicated in [Table L1] to [Table L3](hereinafter, referred to as Compound Class SX₈₁₀).

A compound (L-4) wherein T represents a group represented by T20, R^{3b} represents a hydrogen atom, R^{3c} represents a chlorine atom, and R¹ represents any substituents indicated in [Table L1] to [Table L3](hereinafter, referred to as Compound Class SX₈₁₁).

A compound (L-4) wherein T represents a group represented by T20, R^{3b} represents a hydrogen atom, R^{3c} represents a bromine atom and R¹ represents any substituents indicated in [Table L1] to [Table L3](hereinafter, referred to as Compound Class SX₈₁₂).

A compound (L-4) wherein T represents a group represented by T20, R^{3b} represents a hydrogen atom, R^{3c} represents an iodine atom and R¹ represents any substituents indicated in [Table L1] to [Table L3](hereinafter, referred to as Compound Class SX₈₁₃).

A compound (L-4) wherein T represents a group represented by T21, R^{3b} represents a hydrogen atom, R^{3c} represents a hydrogen atom, and R¹ represents any substituents indicated in [Table L1] to [Table L3](hereinafter, referred to as Compound Class SX₈₁₄).

A compound (L-4) wherein T represents a group represented by T21, R^{3b} represents CF₃, R^{3c} represents a hydrogen atom, and R¹ represents any substituents indicated in [Table L1] to [Table L3](hereinafter, referred to as Compound Class SX₈₁₅).

A compound (L-4) wherein T represents a group represented by T21, R^{3b} represents a chlorine atom, R^{3c} represents a hydrogen atom, and R¹ represents any substituents indicated in [Table L1] to [Table L3](hereinafter, referred to as Compound Class SX₈₁₆).

A compound (L-4) wherein T represents a group represented by T21, R^{3b} represents a bromine atom, R^{3c} represents a hydrogen atom, and R¹ represents any substituents indicated in [Table L1] to [Table L3](hereinafter, referred to as Compound Class SX₈₁₇).

A compound (L-4) wherein T represents a group represented by T21, R^{3b} represents an iodine atom, R^{3c} represents a hydrogen atom, and R¹ represents any substituents indicated in [Table L1] to [Table L3](hereinafter, referred to as Compound Class SX₈₁₈).

A compound (L-4) wherein T represents a group represented by T21, R^{3b} represents a hydrogen atom, R^{3c} represents CF₃, and R¹ represents any substituents indicated in [Table L1] to [Table L3](hereinafter, referred to as Compound Class SX₈₁₉).

A compound (L-4) wherein T represents a group represented by T21, R^{3b} represents a hydrogen atom, R^{3c} represents a chlorine atom, and R¹ represents any substituents indicated in [Table L1] to [Table L3](hereinafter, referred to as Compound Class SX₈₂₀).

A compound (L-4) wherein T represents a group represented by T21, R^{3b} represents a hydrogen atom, R^{3c} represents a bromine atom and R¹ represents any substituents indicated in [Table L1] to [Table L3](hereinafter, referred to as Compound Class SX₈₂₁).

A compound (L-4) wherein T represents a group represented by T21, R^{3b} represents a hydrogen atom, R^{3c} represents an iodine atom and R¹ represents any substituents indicated in [Table L1] to [Table L3](hereinafter, referred to as Compound Class SX₈₂₂).

A compound (L-4) wherein T represents a group represented by T22, R^{3b} represents a hydrogen atom, R^{3c} represents a hydrogen atom, and R¹ represents any substituents indicated in [Table L1] to [Table L3](hereinafter, referred to as Compound Class SX₈₂₃).

A compound (L-4) wherein T represents a group represented by T22, R^{3b} represents CF₃, R^{3c} represents a hydrogen atom, and R¹ represents any substituents indicated in [Table L1] to [Table L3](hereinafter, referred to as Compound Class SX₈₂₄).

A compound (L-4) wherein T represents a group represented by T22, R^{3b} represents a chlorine atom, R^{3c} represents a hydrogen atom, and R¹ represents any substituents indicated in [Table L1] to [Table L3](hereinafter, referred to as Compound Class SX₈₂₅).

A compound (L-4) wherein T represents a group represented by T22, R^{3b} represents a bromine atom, R^{3c} represents a hydrogen atom, and R¹ represents any substituents indicated in [Table L1] to [Table L3](hereinafter, referred to as Compound Class SX₈₂₆).

A compound (L-4) wherein T represents a group represented by T22, R^{3b} represents an iodine atom, R^{3c} represents a hydrogen atom, and R¹ represents any substituents indicated in [Table L1] to [Table L3](hereinafter, referred to as Compound Class SX₈₂₇).

A compound (L-4) wherein T represents a group represented by T22, R^{3b} represents a hydrogen atom, R^{3c} represents CF₃, and R¹ represents any substituents indicated in [Table L1] to [Table L3](hereinafter, referred to as Compound Class SX₈₂₈).

A compound (L-4) wherein T represents a group represented by T22, R^{3b} represents a hydrogen atom, R^{3c} represents a chlorine atom, and R¹ represents any substituents indicated in [Table L1] to [Table L3](hereinafter, referred to as Compound Class SX₈₂₉).

A compound (L-4) wherein T represents a group represented by T22, R^{3b} represents a hydrogen atom, R^{3c} represents a bromine atom and R¹ represents any substituents indicated in [Table L1] to [Table L3](hereinafter, referred to as Compound Class SX₈₃₀).

A compound (L-4) wherein T represents a group represented by T22, R^{3b} represents a hydrogen atom, R^{3c} represents an iodine atom and R¹ represents any substituents indicated in [Table L1] to [Table L3](hereinafter, referred to as Compound Class SX₈₃₁).

A compound (L-4) wherein T represents a group represented by T23, R^{3b} represents a hydrogen atom, R^{3c} represents a hydrogen atom, and R¹ represents any substituents indicated in [Table L1] to [Table L3](hereinafter, referred to as Compound Class SX₈₃₂).

A compound (L-4) wherein T represents a group represented by T23, R^{3b} represents CF₃, R^{3c} represents a hydrogen atom, and R¹ represents any substituents indicated in [Table L1] to [Table L3](hereinafter, referred to as Compound Class SX₈₃₃).

A compound (L-4) wherein T represents a group represented by T23, R^{3b} represents a chlorine atom, R^{3c} represents a hydrogen atom, and R¹ represents any substituents indicated in [Table L1] to [Table L3](hereinafter, referred to as Compound Class SX₈₃₄).

A compound (L-4) wherein T represents a group represented by T23, R^{3b} represents a bromine atom, R^{3c} represents a hydrogen atom, and R¹ represents any substituents indicated in [Table L1] to [Table L3](hereinafter, referred to as Compound Class SX₈₃₅).

A compound (L-4) wherein T represents a group represented by T23, R^{3b} represents an iodine atom, R^{3c} represents a hydrogen atom, and R¹ represents any substituents indicated in [Table L1] to [Table L3](hereinafter, referred to as Compound Class SX₈₃₆).

A compound (L-4) wherein T represents a group represented by T23, R^{3b} represents a hydrogen atom, R^{3c} represents CF₃, and R¹ represents any substituents indicated in [Table L1] to [Table L3](hereinafter, referred to as Compound Class SX₈₃₇).

A compound (L-4) wherein T represents a group represented by T23, R^{3b} represents a hydrogen atom, R^{3c} represents a chlorine atom, and R¹ represents any substituents indicated in [Table L1] to [Table L3](hereinafter, referred to as Compound Class SX₈₃₈).

A compound (L-4) wherein T represents a group represented by T23, R^{3b} represents a hydrogen atom, R^{3c} represents a bromine atom and R¹ represents any substituents indicated in [Table L1] to [Table L3](hereinafter, referred to as Compound Class SX₈₃₉).

A compound (L-4) wherein T represents a group represented by T23, R^{3b} represents a hydrogen atom, R^{3c} represents an iodine atom and R¹ represents any substituents indicated in [Table L1] to [Table L3](hereinafter, referred to as Compound Class SX₈₄₀).

A compound (L-4) wherein T represents a group represented by T24, R^{3b} represents a hydrogen atom, R^{3c} represents a hydrogen atom, and R¹ represents any substituents indicated in [Table L1] to [Table L3](hereinafter, referred to as Compound Class SX₈₄₁).

A compound (L-4) wherein T represents a group represented by T24, R^{3b} represents CF₃, R^{3c} represents a hydrogen atom, and R¹ represents any substituents indicated in [Table L1] to [Table L3](hereinafter, referred to as Compound Class SX₈₄₂).

A compound (L-4) wherein T represents a group represented by T24, R^{3b} represents a chlorine atom, R^{3c} represents a hydrogen atom, and R¹ represents any substituents indicated in [Table L1] to [Table L3](hereinafter, referred to as Compound Class SX₈₄₃).

A compound (L-4) wherein T represents a group represented by T24, R^{3b} represents a bromine atom, R^{3c} represents a hydrogen atom, and R¹ represents any substituents indicated in [Table L1] to [Table L3](hereinafter, referred to as Compound Class SX₈₄₄).

A compound (L-4) wherein T represents a group represented by T24, R^{3b} represents an iodine atom, R^{3c} represents a hydrogen atom, and R¹ represents any substituents indicated in [Table L1] to [Table L3](hereinafter, referred to as Compound Class SX₈₄₅).

A compound (L-4) wherein T represents a group represented by T24, R^{3b} represents a hydrogen atom, R^{3c} represents CF₃, and R¹ represents any substituents indicated in [Table L1] to [Table L3](hereinafter, referred to as Compound Class SX₈₄₆).

A compound (L-4) wherein T represents a group represented by T24, R^{3b} represents a hydrogen atom, R^{3c} represents a chlorine atom, and R¹ represents any substituents indicated in [Table L1] to [Table L3](hereinafter, referred to as Compound Class SX₈₄₇).

A compound (L-4) wherein T represents a group represented by T24, R^{3b} represents a hydrogen atom, R^{3c} represents a bromine atom and R¹ represents any substituents indicated in [Table L1] to [Table L3](hereinafter, referred to as Compound Class SX₈₄₈).

A compound (L-4) wherein T represents a group represented by T24, R^{3b} represents a hydrogen atom, R^{3c} represents an iodine atom and R¹ represents any substituents indicated in [Table L1] to [Table L3](hereinafter, referred to as Compound Class SX₈₄₉).

A compound (L-4) wherein T represents a group represented by T25, R^{3b} represents a hydrogen atom, R^{3c} represents a hydrogen atom, and R¹ represents any substituents indicated in [Table L1] to [Table L3](hereinafter, referred to as Compound Class SX₈₅₀).

A compound (L-4) wherein T represents a group represented by T25, R^{3b} represents CF₃, R^{3c} represents a hydrogen atom, and R¹ represents any substituents indicated in [Table L1] to [Table L3](hereinafter, referred to as Compound Class SX₈₅₁).

A compound (L-4) wherein T represents a group represented by T25, R^{3b} represents a chlorine atom, R^{3c} represents a hydrogen atom, and R¹ represents any substituents indicated in [Table L1] to [Table L3](hereinafter, referred to as Compound Class SX₈₅₂).

A compound (L-4) wherein T represents a group represented by T25, R^{3b} represents a bromine atom, R^{3c} represents a hydrogen atom, and R¹ represents any substituents indicated in [Table L1] to [Table L3](hereinafter, referred to as Compound Class SX₈₅₃).

A compound (L-4) wherein T represents a group represented by T25, R^{3b} represents an iodine atom, R^{3c} represents a hydrogen atom, and R¹ represents any substituents indicated in [Table L1] to [Table L3](hereinafter, referred to as Compound Class SX₈₅₄).

A compound (L-4) wherein T represents a group represented by T25, R^{3b} represents a hydrogen atom, R^{3c} represents CF₃, and R¹ represents any substituents indicated in [Table L1] to [Table L3](hereinafter, referred to as Compound Class SX₈₅₅).

A compound (L-4) wherein T represents a group represented by T25, R^{3b} represents a hydrogen atom, R^{3c} represents a chlorine atom, and R¹ represents any substituents indicated in [Table L1] to [Table L3](hereinafter, referred to as Compound Class SX₈₅₆).

A compound (L-4) wherein T represents a group represented by T25, R^{3b} represents a hydrogen atom, R^{3c} represents a bromine atom and R¹ represents any substituents indicated in [Table L1] to [Table L3](hereinafter, referred to as Compound Class SX₈₅₇).

A compound (L-4) wherein T represents a group represented by T25, R^{3b} represents a hydrogen atom, R^{3c} represents an iodine atom and R¹ represents any substituents indicated in [Table L1] to [Table L3](hereinafter, referred to as Compound Class SX₈₅₈).

A compound (L-4) wherein T represents a group represented by T26, R^{3b} represents a hydrogen atom, R^{3c} represents a hydrogen atom, and R¹ represents any substituents indicated in [Table L1] to [Table L3](hereinafter, referred to as Compound Class SX₈₅₉).

A compound (L-4) wherein T represents a group represented by T26, R^{3b} represents CF₃, R^{3c} represents a hydrogen atom, and R¹ represents any substituents indicated in [Table L1] to [Table L3](hereinafter, referred to as Compound Class SX₈₆₀).

A compound (L-4) wherein T represents a group represented by T26, R^{3b} represents a chlorine atom, R^{3c} represents a hydrogen atom, and R¹ represents any substituents indicated in [Table L1] to [Table L3](hereinafter, referred to as Compound Class SX₈₆₁).

A compound (L-4) wherein T represents a group represented by T26, R^{3b} represents a bromine atom, R^{3c} represents a hydrogen atom, and R¹ represents any substituents indicated in [Table L1] to [Table L3](hereinafter, referred to as Compound Class SX₈₆₂).

A compound (L-4) wherein T represents a group represented by T26, R^{3b} represents an iodine atom, R^{3c} represents a hydrogen atom, and R¹ represents any substituents indicated in [Table L1] to [Table L3](hereinafter, referred to as Compound Class SX₈₆₃).

A compound (L-4) wherein T represents a group represented by T26, R^{3b} represents a hydrogen atom, R^{3c} represents CF₃, and R¹ represents any substituents indicated in [Table L1] to [Table L3](hereinafter, referred to as Compound Class SX₈₆₄).

A compound (L-4) wherein T represents a group represented by T26, R^{3b} represents a hydrogen atom, R^{3c} represents a chlorine atom, and R¹ represents any substituents indicated in [Table L1] to [Table L3](hereinafter, referred to as Compound Class SX₈₆₅).

A compound (L-4) wherein T represents a group represented by T26, R^{3b} represents a hydrogen atom, R^{3c} represents a bromine atom and R¹ represents any substituents indicated in [Table L1] to [Table L3](hereinafter, referred to as Compound Class SX₈₆₆).

A compound (L-4) wherein T represents a group represented by T26, R^{3b} represents a hydrogen atom, R^{3c} represents an iodine atom and R¹ represents any substituents indicated in [Table L1] to [Table L3](hereinafter, referred to as Compound Class SX₈₆₇).

A compound (L-4) wherein T represents a group represented by T27, R^{3b} represents a hydrogen atom, R^{3c} represents a hydrogen atom, and R¹ represents any substituents indicated in [Table L1] to [Table L3](hereinafter, referred to as Compound Class SX₈₆₈).

A compound (L-4) wherein T represents a group represented by T27, R^{3b} represents CF₃, R^{3c} represents a hydrogen atom, and R¹ represents any substituents indicated in [Table L1] to [Table L3](hereinafter, referred to as Compound Class SX₈₆₉).

A compound (L-4) wherein T represents a group represented by T27, R^{3b} represents a chlorine atom, R^{3c} represents a hydrogen atom, and R¹ represents any substituents indicated in [Table L1] to [Table L3](hereinafter, referred to as Compound Class SX₈₇₀).

A compound (L-4) wherein T represents a group represented by T27, R^{3b} represents a bromine atom, R^{3c} represents a hydrogen atom, and R¹ represents any substituents indicated in [Table L1] to [Table L3](hereinafter, referred to as Compound Class SX₈₇₁).

A compound (L-4) wherein T represents a group represented by T27, R^{3b} represents an iodine atom, R^{3c} represents a hydrogen atom, and R¹ represents any substituents indicated in [Table L1] to [Table L3](hereinafter, referred to as Compound Class SX₈₇₂).

A compound (L-4) wherein T represents a group represented by T27, R^{3b} represents a hydrogen atom, R^{3c} represents CF₃, and R¹ represents any substituents indicated in [Table L1] to [Table L3](hereinafter, referred to as Compound Class SX₈₇₃).

A compound (L-4) wherein T represents a group represented by T27, R^{3b} represents a hydrogen atom, R^{3c} represents a chlorine atom, and R¹ represents any substituents indicated in [Table L1] to [Table L3](hereinafter, referred to as Compound Class SX₈₇₄).

A compound (L-4) wherein T represents a group represented by T27, R^{3b} represents a hydrogen atom, R^{3c} represents a bromine atom and R¹ represents any substituents indicated in [Table L1] to [Table L3](hereinafter, referred to as Compound Class SX₈₇₅).

A compound (L-4) wherein T represents a group represented by T27, R^{3b} represents a hydrogen atom, R^{3c} represents an iodine atom and R¹ represents any substituents indicated in [Table L1] to [Table L3](hereinafter, referred to as Compound Class SX₈₇₆).

A compound (L-4) wherein T represents a group represented by T28, R^{3b} represents a hydrogen atom, R^{3c} represents a hydrogen atom, and R¹ represents any substituents indicated in [Table L1] to [Table L3](hereinafter, referred to as Compound Class SX₈₇₇).

A compound (L-4) wherein T represents a group represented by T28, R^{3b} represents CF₃, R^{3c} represents a hydrogen atom, and R¹ represents any substituents indicated in [Table L1] to [Table L3](hereinafter, referred to as Compound Class SX₈₇₈).

A compound (L-4) wherein T represents a group represented by T28, R^{3b} represents a chlorine atom, R^{3c} represents a hydrogen atom, and R¹ represents any substituents indicated in [Table L1] to [Table L3](hereinafter, referred to as Compound Class SX₈₇₉).

A compound (L-4) wherein T represents a group represented by T28, R^{3b} represents a bromine atom, R^{3c} represents a hydrogen atom, and R¹ represents any substituents indicated in [Table L1] to [Table L3](hereinafter, referred to as Compound Class SX₈₈₀).

A compound (L-4) wherein T represents a group represented by T28, R^{3b} represents an iodine atom, R^{3c} represents a hydrogen atom, and R¹ represents any substituents indicated in [Table L1] to [Table L3](hereinafter, referred to as Compound Class SX₈₈₁).

A compound (L-4) wherein T represents a group represented by T28, R^{3b} represents a hydrogen atom, R^{3c} represents CF₃, and R¹ represents any substituents indicated in [Table L1] to [Table L3](hereinafter, referred to as Compound Class SX₈₈₂).

A compound (L-4) wherein T represents a group represented by T28, R^{3b} represents a hydrogen atom, R^{3c} represents a chlorine atom, and R¹ represents any substituents indicated in [Table L1] to [Table L3](hereinafter, referred to as Compound Class SX₈₈₃).

A compound (L-4) wherein T represents a group represented by T28, R^{3b} represents a hydrogen atom, R^{3c} represents a bromine atom and R¹ represents any substituents indicated in [Table L1] to [Table L3](hereinafter, referred to as Compound Class SX₈₈₄).

A compound (L-4) wherein T represents a group represented by T28, R^{3b} represents a hydrogen atom, R^{3c} represents an iodine atom and R¹ represents any substituents indicated in [Table L1] to [Table L3](hereinafter, referred to as Compound Class SX₈₈₅).

A compound (L-4) wherein T represents a group represented by T29, R^{3b} represents a hydrogen atom, R^{3c} represents a hydrogen atom, and R¹ represents any substituents indicated in [Table L1] to [Table L3](hereinafter, referred to as Compound Class SX₈₈₆).

A compound (L-4) wherein T represents a group represented by T29, R^{3b} represents CF₃, R^{3c} represents a hydrogen atom, and R¹ represents any substituents indicated in [Table L1] to [Table L3](hereinafter, referred to as Compound Class SX₈₈₇).

A compound (L-4) wherein T represents a group represented by T29, R^{3b} represents a chlorine atom, R^{3c} represents a hydrogen atom, and R¹ represents any substituents indicated in [Table L1] to [Table L3](hereinafter, referred to as Compound Class SX₈₈₈).

A compound (L-4) wherein T represents a group represented by T29, R^{3b} represents a bromine atom, R^{3c} represents a hydrogen atom, and R¹ represents any substituents indicated in [Table L1] to [Table L3](hereinafter, referred to as Compound Class SX₈₈₉).

A compound (L-4) wherein T represents a group represented by T29, R^{3b} represents an iodine atom, R^{3c} represents a hydrogen atom, and R¹ represents any substituents indicated in [Table L1] to [Table L3](hereinafter, referred to as Compound Class SX₈₉₀).

A compound (L-4) wherein T represents a group represented by T29, R^{3b} represents a hydrogen atom, R^{3c} represents CF₃, and R¹ represents any substituents indicated in [Table L1] to [Table L3](hereinafter, referred to as Compound Class SX₈₉₁).

A compound (L-4) wherein T represents a group represented by T29, R^{3b} represents a hydrogen atom, R^{3c} represents a chlorine atom, and R¹ represents any substituents indicated in [Table L1] to [Table L3](hereinafter, referred to as Compound Class SX₈₉₂).

A compound (L-4) wherein T represents a group represented by T29, R^{3b} represents a hydrogen atom, R^{3c} represents a bromine atom and R¹ represents any substituents indicated in [Table L1] to [Table L3](hereinafter, referred to as Compound Class SX₈₉₃).

A compound (L-4) wherein T represents a group represented by T29, R^{3b} represents a hydrogen atom, R^{3c} represents an iodine atom and R¹ represents any substituents indicated in [Table L1] to [Table L3](hereinafter, referred to as Compound Class SX₈₉₄).

A compound (L-4) wherein T represents a group represented by T30, R^{3b} represents a hydrogen atom, R^{3c} represents a hydrogen atom, and R¹ represents any substituents indicated in [Table L1] to [Table L3](hereinafter, referred to as Compound Class SX₈₉₅).

A compound (L-4) wherein T represents a group represented by T30, R^{3b} represents CF₃, R^{3c} represents a hydrogen atom, and R¹ represents any substituents indicated in [Table L1] to [Table L3](hereinafter, referred to as Compound Class SX₈₉₆).

A compound (L-4) wherein T represents a group represented by T30, R^{3b} represents a chlorine atom, R^{3c} represents a hydrogen atom, and R¹ represents any substituents indicated in [Table L1] to [Table L3](hereinafter, referred to as Compound Class SX₈₉₇).

A compound (L-4) wherein T represents a group represented by T30, R^{3b} represents a bromine atom, R^{3c} represents a hydrogen atom, and R¹ represents any substituents indicated in [Table L1] to [Table L3](hereinafter, referred to as Compound Class SX₈₉₈).

A compound (L-4) wherein T represents a group represented by T30, R^{3b} represents an iodine atom, R^{3c} represents a hydrogen atom, and R¹ represents any substituents indicated in [Table L1] to [Table L3](hereinafter, referred to as Compound Class SX₈₉₉).

A compound (L-4) wherein T represents a group represented by T30, R^{3b} represents a hydrogen atom, R^{3c} represents CF₃, and R¹ represents any substituents indicated in [Table L1] to [Table L3](hereinafter, referred to as Compound Class SX₉₀₀).

A compound (L-4) wherein T represents a group represented by T30, R^{3b} represents a hydrogen atom, R^{3c} represents a chlorine atom, and R¹ represents any substituents indicated in [Table L1] to [Table L3](hereinafter, referred to as Compound Class SX₉₀₁).

A compound (L-4) wherein T represents a group represented by T30, R^{3b} represents a hydrogen atom, R^{3c} represents a bromine atom and R¹ represents any substituents indicated in [Table L1] to [Table L3](hereinafter, referred to as Compound Class SX₉₀₂).

A compound (L-4) wherein T represents a group represented by T30, R^{3b} represents a hydrogen atom, R^{3c} represents an iodine atom and R¹ represents any substituents indicated in [Table L1] to [Table L3](hereinafter, referred to as Compound Class SX₉₀₃).

A compound (L-4) wherein T represents a group represented by T31, R^{3b} represents a hydrogen atom, R^{3c} represents a hydrogen atom, and R¹ represents any substituents indicated in [Table L1] to [Table L3](hereinafter, referred to as Compound Class SX₉₀₄).

A compound (L-4) wherein T represents a group represented by T31, R^{3b} represents CF₃, R^{3c} represents a hydrogen atom, and R¹ represents any substituents indicated in [Table L1] to [Table L3](hereinafter, referred to as Compound Class SX₉₀₅).

A compound (L-4) wherein T represents a group represented by T31, R^{3b} represents a chlorine atom, R^{3c} represents a hydrogen atom, and R¹ represents any substituents indicated in [Table L1] to [Table L3](hereinafter, referred to as Compound Class SX₉₀₆).

A compound (L-4) wherein T represents a group represented by T31, R^{3b} represents a bromine atom, R^{3c} represents a hydrogen atom, and R¹ represents any substituents indicated in [Table L1] to [Table L3](hereinafter, referred to as Compound Class SX₉₀₇).

A compound (L-4) wherein T represents a group represented by T31, R^{3b} represents an iodine atom, R^{3c} represents a hydrogen atom, and R¹ represents any substituents indicated in [Table L1] to [Table L3](hereinafter, referred to as Compound Class SX₉₀₈).

A compound (L-4) wherein T represents a group represented by T31, R^{3b} represents a hydrogen atom, R^{3c} represents CF₃, and R¹ represents any substituents indicated in [Table L1] to [Table L3](hereinafter, referred to as Compound Class SX₉₀₉).

A compound (L-4) wherein T represents a group represented by T31, R^{3b} represents a hydrogen atom, R^{3c} represents a chlorine atom, and R¹ represents any substituents indicated in [Table L1] to [Table L3](hereinafter, referred to as Compound Class SX₉₁₀).

A compound (L-4) wherein T represents a group represented by T31, R^{3b} represents a hydrogen atom, R^{3c} represents a bromine atom and R¹ represents any substituents indicated in [Table L1] to [Table L3](hereinafter, referred to as Compound Class SX₉₁₁).

A compound (L-4) wherein T represents a group represented by T31, R^{3b} represents a hydrogen atom, R^{3c} represents an iodine atom and R¹ represents any substituents indicated in [Table L1] to [Table L3](hereinafter, referred to as Compound Class SX₉₁₂).

A compound represented by formula (L-5):

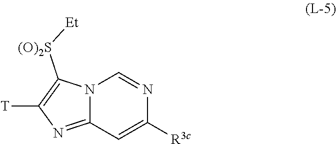

(hereinafter, referred to as compound (L-5)), wherein T represents a group represented by T1, R^{3c} represents a hydrogen atom, and R¹ represents any substituents indicated in [Table L1] to [Table L3](hereinafter, referred to as Compound Class SX₉₁₃).

A compound (L-5) wherein T represents a group represented by T1, R^{3c} represents CF₃, and R¹ represents any substituents indicated in [Table L1] to [Table L3](hereinafter, referred to as Compound Class SX₉₁₄).

A compound (L-5) wherein T represents a group represented by T1, R^{3c} represents a chlorine atom, and R¹ represents any substituents indicated in [Table L1] to [Table L3](hereinafter, referred to as Compound Class SX₉₁₅).

A compound (L-5) wherein T represents a group represented by T1, R^{3c} represents a bromine atom, and R¹ represents any substituents indicated in [Table L1] to [Table L3](hereinafter, referred to as Compound Class SX₉₁₆).

A compound (L-5) wherein T represents a group represented by T1, R^{3c} represents an iodine atom, and R¹ represents any substituents indicated in [Table L1] to [Table L3](hereinafter, referred to as Compound Class SX₉₁₇).

A compound (L-5) wherein T represents a group represented by T2, R^{3c} represents a hydrogen atom, and R¹ represents any substituents indicated in [Table L1] to [Table L3](hereinafter, referred to as Compound Class SX₉₁₈).

A compound (L-5) wherein T represents a group represented by T2, R^{3c} represents CF₃, and R¹ represents any substituents indicated in [Table L1] to [Table L3](hereinafter, referred to as Compound Class SX₉₁₉).

A compound (L-5) wherein T represents a group represented by T2, R^{3c} represents a chlorine atom, and R¹ represents any substituents indicated in [Table L1] to [Table L3](hereinafter, referred to as Compound Class SX₉₂₀).

A compound (L-5) wherein T represents a group represented by T2, R^{3c} represents a bromine atom, and R¹ represents any substituents indicated in [Table L1] to [Table L3](hereinafter, referred to as Compound Class SX₉₂₁).

A compound (L-5) wherein T represents a group represented by T2, R^{3c} represents an iodine atom, and R¹ represents any substituents indicated in [Table L1] to [Table L3](hereinafter, referred to as Compound Class SX₉₂₂).

A compound (L-5) wherein T represents a group represented by T3, R^{3c} represents a hydrogen atom, and R¹ represents any substituents indicated in [Table L1] to [Table L3](hereinafter, referred to as Compound Class SX₉₂₃).

A compound (L-5) wherein T represents a group represented by T3, R^{3c} represents CF₃, and R¹ represents any substituents indicated in [Table L1] to [Table L3](hereinafter, referred to as Compound Class SX₉₂₄).

A compound (L-5) wherein T represents a group represented by T3, R^{3c} represents a chlorine atom, and R¹ represents any substituents indicated in [Table L1] to [Table L3](hereinafter, referred to as Compound Class SX₉₂₅).

A compound (L-5) wherein T represents a group represented by T3, R^{3c} represents a bromine atom, and R¹ represents any substituents indicated in [Table L1] to [Table L3](hereinafter, referred to as Compound Class SX₉₂₆).

A compound (L-5) wherein T represents a group represented by T3, R^{3c} represents an iodine atom, and R¹ represents any substituents indicated in [Table L1] to [Table L3](hereinafter, referred to as Compound Class SX₉₂₇).

A compound (L-5) wherein T represents a group represented by T4, R^{3c} represents a hydrogen atom, and R¹ represents any substituents indicated in [Table L1] to [Table L3](hereinafter, referred to as Compound Class SX₉₂₈).

A compound (L-5) wherein T represents a group represented by T4, R^{3c} represents CF₃, and R¹ represents any substituents indicated in [Table L1] to [Table L3](hereinafter, referred to as Compound Class SX₉₂₉).

A compound (L-5) wherein T represents a group represented by T4, R^{3c} represents a chlorine atom, and R¹ represents any substituents indicated in [Table L1] to [Table L3](hereinafter, referred to as Compound Class SX₉₃₀).

A compound (L-5) wherein T represents a group represented by T4, R^{3c} represents a bromine atom, and R¹ represents any substituents indicated in [Table L1] to [Table L3](hereinafter, referred to as Compound Class SX₉₃₁).

A compound (L-5) wherein T represents a group represented by T4, R^{3c} represents an iodine atom, and R¹ represents any substituents indicated in [Table L1] to [Table L3](hereinafter, referred to as Compound Class SX₉₃₂).

A compound (L-5) wherein T represents a group represented by T5, R^{3c} represents a hydrogen atom, and R¹ represents any substituents indicated in [Table L1] to [Table L3](hereinafter, referred to as Compound Class SX₉₃₃).

A compound (L-5) wherein T represents a group represented by T5, R^{3c} represents CF₃, and R¹ represents any substituents indicated in [Table L1] to [Table L3](hereinafter, referred to as Compound Class SX₉₃₄).

A compound (L-5) wherein T represents a group represented by T5, R^{3c} represents a chlorine atom, and R¹ represents any substituents indicated in [Table L1] to [Table L3](hereinafter, referred to as Compound Class SX₉₃₅).

A compound (L-5) wherein T represents a group represented by T5, R^{3c} represents a bromine atom, and R¹ represents any substituents indicated in [Table L1] to [Table L3](hereinafter, referred to as Compound Class SX₉₃₆).

A compound (L-5) wherein T represents a group represented by T5, R^{3c} represents an iodine atom, and R¹ represents any substituents indicated in [Table L1] to [Table L3](hereinafter, referred to as Compound Class SX₉₃₇).

A compound (L-5) wherein T represents a group represented by T6, R^{3c} represents a hydrogen atom, and R¹ represents any substituents indicated in [Table L1] to [Table L3](hereinafter, referred to as Compound Class SX₉₃₈).

A compound (L-5) wherein T represents a group represented by T6, R^{3c} represents CF₃, and R¹ represents any substituents indicated in [Table L1] to [Table L3](hereinafter, referred to as Compound Class SX₉₃₉).

A compound (L-5) wherein T represents a group represented by T6, R^{3c} represents a chlorine atom, and R¹ represents any substituents indicated in [Table L1] to [Table L3](hereinafter, referred to as Compound Class SX₉₄₀).

A compound (L-5) wherein T represents a group represented by T6, R^{3c} represents a bromine atom, and R¹ represents any substituents indicated in [Table L1] to [Table L3](hereinafter, referred to as Compound Class SX₉₄₁).

A compound (L-5) wherein T represents a group represented by T6, R^{3c} represents an iodine atom, and R¹ represents any substituents indicated in [Table L1] to [Table L3](hereinafter, referred to as Compound Class SX₉₄₂).

A compound (L-5) wherein T represents a group represented by T7, R^{3c} represents a hydrogen atom, and R¹ represents any substituents indicated in [Table L1] to [Table L3](hereinafter, referred to as Compound Class SX₉₄₃).

A compound (L-5) wherein T represents a group represented by T7, R^{3c} represents CF₃, and R¹ represents any substituents indicated in [Table L1] to [Table L3](hereinafter, referred to as Compound Class SX₉₄₄).

A compound (L-5) wherein T represents a group represented by T7, R^{3c} represents a chlorine atom, and R¹ represents any substituents indicated in [Table L1] to [Table L3](hereinafter, referred to as Compound Class SX₉₄₅).

A compound (L-5) wherein T represents a group represented by T7, R^{3c} represents a bromine atom, and R¹ represents any substituents indicated in [Table L1] to [Table L3](hereinafter, referred to as Compound Class SX₉₄₆).

A compound (L-5) wherein T represents a group represented by T7, R^{3c} represents an iodine atom, and R¹ represents any substituents indicated in [Table L1] to [Table L3](hereinafter, referred to as Compound Class SX₉₄₇).

A compound (L-5) wherein T represents a group represented by T8, R^{3c} represents a hydrogen atom, and R¹ represents any substituents indicated in [Table L1] to [Table L3](hereinafter, referred to as Compound Class SX₉₄₈).

A compound (L-5) wherein T represents a group represented by T8, R^{3c} represents CF₃, and R¹ represents any substituents indicated in [Table L1] to [Table L3](hereinafter, referred to as Compound Class SX₉₄₉).

A compound (L-5) wherein T represents a group represented by T8, R^{3c} represents a chlorine atom, and R¹ represents any substituents indicated in [Table L1] to [Table L3](hereinafter, referred to as Compound Class SX₉₅₀).

A compound (L-5) wherein T represents a group represented by T8, R^{3c} represents a bromine atom, and R¹ represents any substituents indicated in [Table L1] to [Table L3](hereinafter, referred to as Compound Class SX₉₅₁).

A compound (L-5) wherein T represents a group represented by T8, R^{3c} represents an iodine atom, and R¹ represents any substituents indicated in [Table L1] to [Table L3](hereinafter, referred to as Compound Class SX₉₅₂).

A compound (L-5) wherein T represents a group represented by T9, R^{3c} represents a hydrogen atom, and R¹ represents any substituents indicated in [Table L1] to [Table L3](hereinafter, referred to as Compound Class SX₉₅₃).

A compound (L-5) wherein T represents a group represented by T9, R^{3c} represents CF₃, and R¹ represents any substituents indicated in [Table L1] to [Table L3](hereinafter, referred to as Compound Class SX₉₅₄).

A compound (L-5) wherein T represents a group represented by T9, R^{3c} represents a chlorine atom, and R¹ represents any substituents indicated in [Table L1] to [Table L3](hereinafter, referred to as Compound Class SX₉₅₅).

A compound (L-5) wherein T represents a group represented by T9, R^{3c} represents a bromine atom, and R¹ represents any substituents indicated in [Table L1] to [Table L3](hereinafter, referred to as Compound Class SX₉₅₆).

A compound (L-5) wherein T represents a group represented by T9, R^{3c} represents an iodine atom, and R¹ represents any substituents indicated in [Table L1] to [Table L3](hereinafter, referred to as Compound Class SX₉₅₇).

A compound (L-5) wherein T represents a group represented by T10, R^{3c} represents a hydrogen atom, and R¹ represents any substituents indicated in [Table L1] to [Table L3](hereinafter, referred to as Compound Class SX₉₅₈).

A compound (L-5) wherein T represents a group represented by T10, R^{3c} represents CF₃, and R¹ represents any substituents indicated in [Table L1] to [Table L3](hereinafter, referred to as Compound Class SX₉₅₉).

A compound (L-5) wherein T represents a group represented by T10, R^{3c} represents a chlorine atom, and R¹ represents any substituents indicated in [Table L1] to [Table L3](hereinafter, referred to as Compound Class SX₉₆₀).

A compound (L-5) wherein T represents a group represented by T10, R^{3c} represents a bromine atom, and R¹ represents any substituents indicated in [Table L1] to [Table L3](hereinafter, referred to as Compound Class SX₉₆₁).

A compound (L-5) wherein T represents a group represented by T10, R^{3c} represents an iodine atom, and R¹ represents any substituents indicated in [Table L1] to [Table L3](hereinafter, referred to as Compound Class SX₉₆₂).

A compound (L-5) wherein T represents a group represented by T1l, R^{3c} represents a hydrogen atom, and R¹ represents any substituents indicated in [Table L1] to [Table L3](hereinafter, referred to as Compound Class SX₉₆₃).

A compound (L-5) wherein T represents a group represented by T1l, R^{3c} represents CF₃, and R¹ represents any substituents indicated in [Table L1] to [Table L3](hereinafter, referred to as Compound Class SX₉₆₄).

A compound (L-5) wherein T represents a group represented by T1l, R^{3c} represents a chlorine atom, and R¹ represents any substituents indicated in [Table L1] to [Table L3](hereinafter, referred to as Compound Class SX₉₆₅).

A compound (L-5) wherein T represents a group represented by T1l, R^{3c} represents a bromine atom, and R¹ represents any substituents indicated in [Table L1] to [Table L3](hereinafter, referred to as Compound Class SX₉₆₆).

A compound (L-5) wherein T represents a group represented by T1l, R^{3c} represents an iodine atom, and R¹ represents any substituents indicated in [Table L1] to [Table L3](hereinafter, referred to as Compound Class SX₉₆₇).

A compound (L-5) wherein T represents a group represented by T12, R^{3c} represents a hydrogen atom, and R¹ represents any substituents indicated in [Table L1] to [Table L3](hereinafter, referred to as Compound Class SX₉₆₈).

A compound (L-5) wherein T represents a group represented by T12, R^{3c} represents CF₃, and R¹ represents any substituents indicated in [Table L1] to [Table L3](hereinafter, referred to as Compound Class SX₉₆₉).

A compound (L-5) wherein T represents a group represented by T12, R^{3c} represents a chlorine atom, and R¹ represents any substituents indicated in [Table L1] to [Table L3](hereinafter, referred to as Compound Class SX₉₇₀).

A compound (L-5) wherein T represents a group represented by T12, R^{3c} represents a bromine atom, and R¹ represents any substituents indicated in [Table L1] to [Table L3](hereinafter, referred to as Compound Class SX₉₇₁).

A compound (L-5) wherein T represents a group represented by T12, R^{3c} represents an iodine atom, and R¹ represents any substituents indicated in [Table L1] to [Table L3](hereinafter, referred to as Compound Class SX₉₇₂).

A compound (L-5) wherein T represents a group represented by T13, R^{3c} represents a hydrogen atom, and R¹ represents any substituents indicated in [Table L1] to [Table L3](hereinafter, referred to as Compound Class SX₉₇₃).

A compound (L-5) wherein T represents a group represented by T13, R^{3c} represents CF₃, and R¹ represents any substituents indicated in [Table L1] to [Table L3](hereinafter, referred to as Compound Class SX₉₇₄).

A compound (L-5) wherein T represents a group represented by T13, R^{3c} represents a chlorine atom, and R¹ represents any substituents indicated in [Table L1] to [Table L3](hereinafter, referred to as Compound Class SX₉₇₅).

A compound (L-5) wherein T represents a group represented by T13, R^{3c} represents a bromine atom, and R¹ represents any substituents indicated in [Table L1] to [Table L3](hereinafter, referred to as Compound Class SX₉₇₆).

A compound (L-5) wherein T represents a group represented by T13, R^{3c} represents an iodine atom, and R¹ represents any substituents indicated in [Table L1] to [Table L3](hereinafter, referred to as Compound Class SX₉₇₇).

A compound (L-5) wherein T represents a group represented by T14, R^{3c} represents a hydrogen atom, and R¹ represents any substituents indicated in [Table L1] to [Table L3](hereinafter, referred to as Compound Class SX₉₇₈).

A compound (L-5) wherein T represents a group represented by T14, R^{3c} represents CF₃, and R¹ represents any substituents indicated in [Table L1] to [Table L3](hereinafter, referred to as Compound Class SX₉₇₉).

A compound (L-5) wherein T represents a group represented by T14, R^{3c} represents a chlorine atom, and R¹ represents any substituents indicated in [Table L1] to [Table L3](hereinafter, referred to as Compound Class SX₉₈₀).

A compound (L-5) wherein T represents a group represented by T14, R^{3c} represents a bromine atom, and R¹ represents any substituents indicated in [Table L1] to [Table L3](hereinafter, referred to as Compound Class SX₉₈₁).

A compound (L-5) wherein T represents a group represented by T14, R^{3c} represents an iodine atom, and R¹ represents any substituents indicated in [Table L1] to [Table L3](hereinafter, referred to as Compound Class SX₉₈₂).

A compound (L-5) wherein T represents a group represented by T15, R^{3c} represents a hydrogen atom, and R¹ represents any substituents indicated in [Table L1] to [Table L3](hereinafter, referred to as Compound Class SX₉₈₃).

A compound (L-5) wherein T represents a group represented by T15, R^{3c} represents CF₃, and R¹ represents any substituents indicated in [Table L1] to [Table L3](hereinafter, referred to as Compound Class SX₉₈₄).

A compound (L-5) wherein T represents a group represented by T15, R^{3c} represents a chlorine atom, and R¹ represents any substituents indicated in [Table L1] to [Table L3](hereinafter, referred to as Compound Class SX₉₈₅).

A compound (L-5) wherein T represents a group represented by T15, R^{3c} represents a bromine atom, and R¹ represents any substituents indicated in [Table L1] to [Table L3](hereinafter, referred to as Compound Class SX₉₈₆).

A compound (L-5) wherein T represents a group represented by T15, R^{3c} represents an iodine atom, and R¹ represents any substituents indicated in [Table L1] to [Table L3](hereinafter, referred to as Compound Class SX₉₈₇).

A compound (L-5) wherein T represents a group represented by T16, R^{3c} represents a hydrogen atom, and R¹ represents any substituents indicated in [Table L1] to [Table L3](hereinafter, referred to as Compound Class SX₉₈₈).

A compound (L-5) wherein T represents a group represented by T16, R^{3c} represents CF₃, and R¹ represents any substituents indicated in [Table L1] to [Table L3](hereinafter, referred to as Compound Class SX₉₈₉).

A compound (L-5) wherein T represents a group represented by T16, R^{3c} represents a chlorine atom, and R¹ represents any substituents indicated in [Table L1] to [Table L3](hereinafter, referred to as Compound Class SX₉₉₀).

A compound (L-5) wherein T represents a group represented by T16, R^{3c} represents a bromine atom, and R¹ represents any substituents indicated in [Table L1] to [Table L3](hereinafter, referred to as Compound Class SX₉₉₁).

A compound (L-5) wherein T represents a group represented by T16, R^{3c} represents an iodine atom, and R¹ represents any substituents indicated in [Table L1] to [Table L3](hereinafter, referred to as Compound Class SX₉₉₂).

A compound (L-5) wherein T represents a group represented by T17, R^{3c} represents a hydrogen atom, and R¹ represents any substituents indicated in [Table L1] to [Table L3](hereinafter, referred to as Compound Class SX₉₉₃).

A compound (L-5) wherein T represents a group represented by T17, R^{3c} represents CF₃, and R¹ represents any substituents indicated in [Table L1] to [Table L3](hereinafter, referred to as Compound Class SX₉₉₄).

A compound (L-5) wherein T represents a group represented by T17, R^{3c} represents a chlorine atom, and R¹ represents any substituents indicated in [Table L1] to [Table L3](hereinafter, referred to as Compound Class SX₉₉₅).

A compound (L-5) wherein T represents a group represented by T17, R^{3c} represents a bromine atom, and R¹ represents any substituents indicated in [Table L1] to [Table L3](hereinafter, referred to as Compound Class SX₉₉₆).

A compound (L-5) wherein T represents a group represented by T17, R^{3c} represents an iodine atom, and R¹ represents any substituents indicated in [Table L1] to [Table L3](hereinafter, referred to as Compound Class SX₉₉₇).

A compound (L-5) wherein T represents a group represented by T18, R^{3c} represents a hydrogen atom, and R¹ represents any substituents indicated in [Table L1] to [Table L3](hereinafter, referred to as Compound Class SX₉₉₈).

A compound (L-5) wherein T represents a group represented by T18, R^{3c} represents CF₃, and R¹ represents any substituents indicated in [Table L1] to [Table L3](hereinafter, referred to as Compound Class SX₉₉₉).

A compound (L-5) wherein T represents a group represented by T18, R^{3c} represents a chlorine atom, and R¹ represents any substituents indicated in [Table L1] to [Table L3](hereinafter, referred to as Compound Class SX₁₀₀₀).

A compound (L-5) wherein T represents a group represented by T18, R^{3c} represents a bromine atom, and R¹ represents any substituents indicated in [Table L1] to [Table L3](hereinafter, referred to as Compound Class SX₁₀₀₁).

A compound (L-5) wherein T represents a group represented by T18, R^{3c} represents an iodine atom, and R¹ represents any substituents indicated in [Table L1] to [Table L3](hereinafter, referred to as Compound Class SX₁₀₀₂).

A compound (L-5) wherein T represents a group represented by T19, R^{3c} represents a hydrogen atom, and R¹ represents any substituents indicated in [Table L1] to [Table L3](hereinafter, referred to as Compound Class SX₁₀₀₃).

A compound (L-5) wherein T represents a group represented by T19, R^{3c} represents CF₃, and R¹ represents any substituents indicated in [Table L1] to [Table L3](hereinafter, referred to as Compound Class SX₁₀₀₄).

A compound (L-5) wherein T represents a group represented by T19, R^{3c} represents a chlorine atom, and R¹ represents any substituents indicated in [Table L1] to [Table L3](hereinafter, referred to as Compound Class SX₁₀₀₅).

A compound (L-5) wherein T represents a group represented by T19, R^{3c} represents a bromine atom, and R¹ represents any substituents indicated in [Table L1] to [Table L3](hereinafter, referred to as Compound Class SX₁₀₀₆).

A compound (L-5) wherein T represents a group represented by T19, R^{3c} represents an iodine atom, and R¹ represents any substituents indicated in [Table L1] to [Table L3](hereinafter, referred to as Compound Class SX₁₀₀₇).

A compound (L-5) wherein T represents a group represented by T20, R^{3c} represents a hydrogen atom, and R¹ represents any substituents indicated in [Table L1] to [Table L3](hereinafter, referred to as Compound Class SX₁₀₀₈).

A compound (L-5) wherein T represents a group represented by T20, R^{3c} represents CF₃, and R¹ represents any substituents indicated in [Table L1] to [Table L3](hereinafter, referred to as Compound Class SX₁₀₀₉).

A compound (L-5) wherein T represents a group represented by T20, R^{3c} represents a chlorine atom, and R¹ represents any substituents indicated in [Table L1] to [Table L3](hereinafter, referred to as Compound Class SX₁₀₁₀).

A compound (L-5) wherein T represents a group represented by T20, R^{3c} represents a bromine atom, and R¹ represents any substituents indicated in [Table L1] to [Table L3](hereinafter, referred to as Compound Class SX₁₀₁ₗ).

A compound (L-5) wherein T represents a group represented by T20, R^{3c} represents an iodine atom, and R¹ represents any substituents indicated in [Table L1] to [Table L3](hereinafter, referred to as Compound Class SX₁₀₁₂).

A compound (L-5) wherein T represents a group represented by T21, R^{3c} represents a hydrogen atom, and R¹ represents any substituents indicated in [Table L1] to [Table L3](hereinafter, referred to as Compound Class SX₁₀₁₃).

A compound (L-5) wherein T represents a group represented by T21, R^{3c} represents CF₃, and R¹ represents any substituents indicated in [Table L1] to [Table L3](hereinafter, referred to as Compound Class SX₁₀₁₄).

A compound (L-5) wherein T represents a group represented by T21, R^{3c} represents a chlorine atom, and R¹ represents any substituents indicated in [Table L1] to [Table L3](hereinafter, referred to as Compound Class SX₁₀₁₅).

A compound (L-5) wherein T represents a group represented by T21, R^{3c} represents a bromine atom, and R¹ represents any substituents indicated in [Table L1] to [Table L3](hereinafter, referred to as Compound Class SX₁₀₁₆).

A compound (L-5) wherein T represents a group represented by T21, R^{3c} represents an iodine atom, and R¹ represents any substituents indicated in [Table L1] to [Table L3](hereinafter, referred to as Compound Class SX₁₀₁₇).

A compound (L-5) wherein T represents a group represented by T22, R^{3c} represents a hydrogen atom, and R¹ represents any substituents indicated in [Table L1] to [Table L3](hereinafter, referred to as Compound Class SX₁₀₁₈).

A compound (L-5) wherein T represents a group represented by T22, R^{3c} represents CF₃, and R¹ represents any substituents indicated in [Table L1] to [Table L3](hereinafter, referred to as Compound Class SX₁₀₁₉).

A compound (L-5) wherein T represents a group represented by T22, R^{3c} represents a chlorine atom, and R¹ represents any substituents indicated in [Table L1] to [Table L3](hereinafter, referred to as Compound Class SX₁₀₂₀).

A compound (L-5) wherein T represents a group represented by T22, R^{3c} represents a bromine atom, and R¹ represents any substituents indicated in [Table L1] to [Table L3](hereinafter, referred to as Compound Class SX₁₀₂₁).

A compound (L-5) wherein T represents a group represented by T22, R^{3c} represents an iodine atom, and R¹ represents any substituents indicated in [Table L1] to [Table L3](hereinafter, referred to as Compound Class SX₁₀₂₂).

A compound (L-5) wherein T represents a group represented by T23, R^{3c} represents a hydrogen atom, and R¹ represents any substituents indicated in [Table L1] to [Table L3](hereinafter, referred to as Compound Class SX₁₀₂₃).

A compound (L-5) wherein T represents a group represented by T23, R^{3c} represents CF₃, and R¹ represents any substituents indicated in [Table L1] to [Table L3](hereinafter, referred to as Compound Class SX₁₀₂₄).

A compound (L-5) wherein T represents a group represented by T23, R^{3c} represents a chlorine atom, and R¹ represents any substituents indicated in [Table L1] to [Table L3](hereinafter, referred to as Compound Class SX₁₀₂₅).

A compound (L-5) wherein T represents a group represented by T23, R^{3c} represents a bromine atom, and R¹ represents any substituents indicated in [Table L1] to [Table L3](hereinafter, referred to as Compound Class SX₁₀₂₆).

A compound (L-5) wherein T represents a group represented by T23, R^{3c} represents an iodine atom, and R¹ represents any substituents indicated in [Table L1] to [Table L3](hereinafter, referred to as Compound Class SX₁₀₂₇).

A compound (L-5) wherein T represents a group represented by T24, R^{3c} represents a hydrogen atom, and R¹ represents any substituents indicated in [Table L1] to [Table L3](hereinafter, referred to as Compound Class SX₁₀₂₈).

A compound (L-5) wherein T represents a group represented by T24, R^{3c} represents CF₃, and R¹ represents any substituents indicated in [Table L1] to [Table L3](hereinafter, referred to as Compound Class SX₁₀₂₉).

A compound (L-5) wherein T represents a group represented by T24, R^{3c} represents a chlorine atom, and R¹ represents any substituents indicated in [Table L1] to [Table L3](hereinafter, referred to as Compound Class SX₁₀₃₀).

A compound (L-5) wherein T represents a group represented by T24, R^{3c} represents a bromine atom, and R¹ represents any substituents indicated in [Table L1] to [Table L3](hereinafter, referred to as Compound Class SX₁₀₃₁).

A compound (L-5) wherein T represents a group represented by T24, R^{3c} represents an iodine atom, and R¹ represents any substituents indicated in [Table L1] to [Table L3](hereinafter, referred to as Compound Class SX₁₀₃₂).

A compound (L-5) wherein T represents a group represented by T25, R^{3c} represents a hydrogen atom, and R¹ represents any substituents indicated in [Table L1] to [Table L3](hereinafter, referred to as Compound Class SX₁₀₃₃).

A compound (L-5) wherein T represents a group represented by T25, R^{3c} represents CF₃, and R¹ represents any substituents indicated in [Table L1] to [Table L3](hereinafter, referred to as Compound Class SX₁₀₃₄).

A compound (L-5) wherein T represents a group represented by T25, R^{3c} represents a chlorine atom, and R¹ represents any substituents indicated in [Table L1] to [Table L3](hereinafter, referred to as Compound Class SX₁₀₃₅).

A compound (L-5) wherein T represents a group represented by T25, R^{3c} represents a bromine atom, and R¹ represents any substituents indicated in [Table L1] to [Table L3](hereinafter, referred to as Compound Class SX₁₀₃₆).

A compound (L-5) wherein T represents a group represented by T25, R^{3c} represents an iodine atom, and R¹ represents any substituents indicated in [Table L1] to [Table L3](hereinafter, referred to as Compound Class SX₁₀₃₇).

A compound (L-5) wherein T represents a group represented by T26, R^{3c} represents a hydrogen atom, and R¹ represents any substituents indicated in [Table L1] to [Table L3](hereinafter, referred to as Compound Class SX₁₀₃₈).

A compound (L-5) wherein T represents a group represented by T26, R^{3c} represents CF₃, and R¹ represents any substituents indicated in [Table L1] to [Table L3](hereinafter, referred to as Compound Class SX₁₀₃₉).

A compound (L-5) wherein T represents a group represented by T26, R^{3c} represents a chlorine atom, and R¹ represents any substituents indicated in [Table L1] to [Table L3](hereinafter, referred to as Compound Class SX₁₀₄₀).

A compound (L-5) wherein T represents a group represented by T26, R^{3c} represents a bromine atom, and R¹ represents any substituents indicated in [Table L1] to [Table L3](hereinafter, referred to as Compound Class SX₁₀₄₁).

A compound (L-5) wherein T represents a group represented by T26, R^{3c} represents an iodine atom, and R¹ represents any substituents indicated in [Table L1] to [Table L3](hereinafter, referred to as Compound Class SX₁₀₄₂).

A compound (L-5) wherein T represents a group represented by T27, R^{3c} represents a hydrogen atom, and R¹ represents any substituents indicated in [Table L1] to [Table L3](hereinafter, referred to as Compound Class SX₁₀₄₃).

A compound (L-5) wherein T represents a group represented by T27, R^{3c} represents CF₃, and R¹ represents any substituents indicated in [Table L1] to [Table L3](hereinafter, referred to as Compound Class SX₁₀₄₄).

A compound (L-5) wherein T represents a group represented by T27, R^{3c} represents a chlorine atom, and R¹ represents any substituents indicated in [Table L1] to [Table L3](hereinafter, referred to as Compound Class SX₁₀₄₅).

A compound (L-5) wherein T represents a group represented by T27, R^{3c} represents a bromine atom, and R¹ represents any substituents indicated in [Table L1] to [Table L3](hereinafter, referred to as Compound Class SX₁₀₄₆).

A compound (L-5) wherein T represents a group represented by T27, R^{3c} represents an iodine atom, and R¹ represents any substituents indicated in [Table L1] to [Table L3](hereinafter, referred to as Compound Class SX₁₀₄₇).

A compound (L-5) wherein T represents a group represented by T28, R^{3c} represents a hydrogen atom, and R¹ represents any substituents indicated in [Table L1] to [Table L3](hereinafter, referred to as Compound Class SX₁₀₄₈).

A compound (L-5) wherein T represents a group represented by T28, R^{3c} represents CF₃, and R¹ represents any substituents indicated in [Table L1] to [Table L3](hereinafter, referred to as Compound Class SX₁₀₄₉).

A compound (L-5) wherein T represents a group represented by T28, R^{3c} represents a chlorine atom, and R¹ represents any substituents indicated in [Table L1] to [Table L3](hereinafter, referred to as Compound Class SX₁₀₅₀).

A compound (L-5) wherein T represents a group represented by T28, R^{3c} represents a bromine atom, and R¹ represents any substituents indicated in [Table L1] to [Table L3](hereinafter, referred to as Compound Class SX₁₀₅₁).

A compound (L-5) wherein T represents a group represented by T28, R^{3c} represents an iodine atom, and R¹ represents any substituents indicated in [Table L1] to [Table L3](hereinafter, referred to as Compound Class SX₁₀₅₂).

A compound (L-5) wherein T represents a group represented by T29, R^{3c} represents a hydrogen atom, and R¹ represents any substituents indicated in [Table L1] to [Table L3](hereinafter, referred to as Compound Class SX₁₀₅₃).

A compound (L-5) wherein T represents a group represented by T29, R^{3c} represents CF₃, and R¹ represents any substituents indicated in [Table L1] to [Table L3](hereinafter, referred to as Compound Class SX₁₀₅₄).

A compound (L-5) wherein T represents a group represented by T29, R^{3c} represents a chlorine atom, and R¹ represents any substituents indicated in [Table L1] to [Table L3](hereinafter, referred to as Compound Class SX₁₀₅₅).

A compound (L-5) wherein T represents a group represented by T29, R^{3c} represents a bromine atom, and R¹ represents any substituents indicated in [Table L1] to [Table L3](hereinafter, referred to as Compound Class SX₁₀₅₆).

A compound (L-5) wherein T represents a group represented by T29, R^{3c} represents an iodine atom, and R¹ represents any substituents indicated in [Table L1] to [Table L3](hereinafter, referred to as Compound Class SX₁₀₅₇).

A compound (L-5) wherein T represents a group represented by T30, R^{3c} represents a hydrogen atom, and R¹ represents any substituents indicated in [Table L1] to [Table L3](hereinafter, referred to as Compound Class SX₁₀₅₈).

A compound (L-5) wherein T represents a group represented by T30, R^{3c} represents CF₃, and R¹ represents any substituents indicated in [Table L1] to [Table L3](hereinafter, referred to as Compound Class SX₁₀₅₉).

A compound (L-5) wherein T represents a group represented by T30, R^{3c} represents a chlorine atom, and R¹ represents any substituents indicated in [Table L1] to [Table L3](hereinafter, referred to as Compound Class SX₁₀₆₀).

A compound (L-5) wherein T represents a group represented by T30, R^{3c} represents a bromine atom, and R¹ represents any substituents indicated in [Table L1] to [Table L3](hereinafter, referred to as Compound Class SX₁₀₆₁).

A compound (L-5) wherein T represents a group represented by T30, R^{3c} represents an iodine atom, and R¹ represents any substituents indicated in [Table L1] to [Table L3](hereinafter, referred to as Compound Class SX₁₀₆₂).

A compound (L-5) wherein T represents a group represented by T31, R^{3c} represents a hydrogen atom, and R¹ represents any substituents indicated in [Table L1] to [Table L3](hereinafter, referred to as Compound Class SX₁₀₆₃).

A compound (L-5) wherein T represents a group represented by T31, R^{3c} represents CF₃, and R¹ represents any substituents indicated in [Table L1] to [Table L3](hereinafter, referred to as Compound Class SX₁₀₆₄).

A compound (L-5) wherein T represents a group represented by T31, R^{3c} represents a chlorine atom, and R¹ represents any substituents indicated in [Table L1] to [Table L3](hereinafter, referred to as Compound Class SX₁₀₆₅).

A compound (L-5) wherein T represents a group represented by T31, R^{3c} represents a bromine atom, and R¹ represents any substituents indicated in [Table L1] to [Table L3](hereinafter, referred to as Compound Class SX₁₀₆₆).

A compound (L-5) wherein T represents a group represented by T31, R^{3c} represents an iodine atom, and R¹ represents any substituents indicated in [Table L1] to [Table L3](hereinafter, referred to as Compound Class SX₁₀₆₇).

A compound represented by formula (L-6):

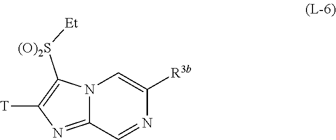

(hereinafter, referred to as compound (L-6)), wherein T represents a group represented by T1, R^{3b} represents a hydrogen atom, and R¹ represents any substituents indicated in [Table L1] to [Table L3](hereinafter, referred to as Compound Class SX₁₀₆₈).

A compound (L-6) wherein T represents a group represented by T1, R^{3b} represents CF₃, and R¹ represents any substituents indicated in [Table L1] to [Table L3](hereinafter, referred to as Compound Class SX₁₀₆₉).

A compound (L-6) wherein T represents a group represented by T1, R^{3b} represents a chlorine atom, and R¹ represents any substituents indicated in [Table L1] to [Table L3](hereinafter, referred to as Compound Class SX₁₀₇₀).

A compound (L-6) wherein T represents a group represented by T1, R^{3b} represents a bromine atom, and R¹ represents any substituents indicated in [Table L1] to [Table L3](hereinafter, referred to as Compound Class SX₁₀₇₁).

A compound (L-6) wherein T represents a group represented by T1, R^{3b} represents an iodine atom, and R¹ represents any substituents indicated in [Table L1] to [Table L3](hereinafter, referred to as Compound Class SX₁₀₇₂).

A compound (L-6), wherein T represents a group represented by T2, R^{3b} represents a hydrogen atom, and R¹ represents any substituents indicated in [Table L1] to [Table L3](hereinafter, referred to as Compound Class SX₁₀₇₃).

A compound (L-6) wherein T represents a group represented by T2, R^{3b} represents CF₃, and R¹ represents any substituents indicated in [Table L1] to [Table L3](hereinafter, referred to as Compound Class SX₁₀₇₄).

A compound (L-6) wherein T represents a group represented by T2, R^{3b} represents a chlorine atom, and R¹ represents any substituents indicated in [Table L1] to [Table L3](hereinafter, referred to as Compound Class SX₁₀₇₅).

A compound (L-6) wherein T represents a group represented by T2, R^{3b} represents a bromine atom, and R¹ represents any substituents indicated in [Table L1] to [Table L3](hereinafter, referred to as Compound Class SX₁₀₇₆).

A compound (L-6) wherein T represents a group represented by T2, R^{3b} represents an iodine atom, and R¹ represents any substituents indicated in [Table L1] to [Table L3](hereinafter, referred to as Compound Class SX₁₀₇₇).

A compound (L-6), wherein T represents a group represented by T3, R^{3b} represents a hydrogen atom, and R¹ represents any substituents indicated in [Table L1] to [Table L3](hereinafter, referred to as Compound Class SX₁₀₇₈).

A compound (L-6) wherein T represents a group represented by T3, R^{3b} represents CF₃, and R¹ represents any substituents indicated in [Table L1] to [Table L3](hereinafter, referred to as Compound Class SX₁₀₇₉).

A compound (L-6) wherein T represents a group represented by T3, R^{3b} represents a chlorine atom, and R¹ represents any substituents indicated in [Table L1] to [Table L3](hereinafter, referred to as Compound Class SX₁₀₈₀).

A compound (L-6) wherein T represents a group represented by T3, R^{3b} represents a bromine atom, and R¹ represents any substituents indicated in [Table L1] to [Table L3](hereinafter, referred to as Compound Class SX₁₀₈₁).

A compound (L-6) wherein T represents a group represented by T3, R^{3b} represents an iodine atom, and R¹ represents any substituents indicated in [Table L1] to [Table L3](hereinafter, referred to as Compound Class SX₁₀₈₂).

A compound (L-6), wherein T represents a group represented by T4, R^{3b} represents a hydrogen atom, and R¹ represents any substituents indicated in [Table L1] to [Table L3](hereinafter, referred to as Compound Class SX₁₀₈₃).

A compound (L-6) wherein T represents a group represented by T4, R^{3b} represents CF₃, and R¹ represents any substituents indicated in [Table L1] to [Table L3](hereinafter, referred to as Compound Class SX₁₀₈₄).

A compound (L-6) wherein T represents a group represented by T4, R^{3b} represents a chlorine atom, and R¹ represents any substituents indicated in [Table L1] to [Table L3](hereinafter, referred to as Compound Class SX₁₀₈₅).

A compound (L-6) wherein T represents a group represented by T4, R^{3b} represents a bromine atom, and R¹ represents any substituents indicated in [Table L1] to [Table L3](hereinafter, referred to as Compound Class SX₁₀₈₆).

A compound (L-6) wherein T represents a group represented by T4, R^{3b} represents an iodine atom, and R¹ represents any substituents indicated in [Table L1] to [Table L3](hereinafter, referred to as Compound Class SX₁₀₈₇).

A compound (L-6), wherein T represents a group represented by T5, R^{3b} represents a hydrogen atom, and R¹ represents any substituents indicated in [Table L1] to [Table L3](hereinafter, referred to as Compound Class SX₁₀₈₈).

A compound (L-6) wherein T represents a group represented by T5, R^{3b} represents CF₃, and R¹ represents any substituents indicated in [Table L1] to [Table L3](hereinafter, referred to as Compound Class SX₁₀₈₉).

A compound (L-6) wherein T represents a group represented by T5, R^{3b} represents a chlorine atom, and R¹ represents any substituents indicated in [Table L1] to [Table L3](hereinafter, referred to as Compound Class SX₁₀₉₀).

A compound (L-6) wherein T represents a group represented by T5, R^{3b} represents a bromine atom, and R¹ represents any substituents indicated in [Table L1] to [Table L3](hereinafter, referred to as Compound Class SX₁₀₉₁).

A compound (L-6) wherein T represents a group represented by T5, R^{3b} represents an iodine atom, and R¹ represents any substituents indicated in [Table L1] to [Table L3](hereinafter, referred to as Compound Class SX₁₀₉₂).

A compound (L-6), wherein T represents a group represented by T6, R^{3b} represents a hydrogen atom, and R¹ represents any substituents indicated in [Table L1] to [Table L3](hereinafter, referred to as Compound Class SX₁₀₉₃).

A compound (L-6) wherein T represents a group represented by T6, R^{3b} represents CF₃, and R¹ represents any substituents indicated in [Table L1] to [Table L3](hereinafter, referred to as Compound Class SX₁₀₉₄).

A compound (L-6) wherein T represents a group represented by T6, R^{3b} represents a chlorine atom, and R¹ represents any substituents indicated in [Table L1] to [Table L3](hereinafter, referred to as Compound Class SX₁₀₉₅).

A compound (L-6) wherein T represents a group represented by T6, R^{3b} represents a bromine atom, and R¹ represents any substituents indicated in [Table L1] to [Table L3](hereinafter, referred to as Compound Class SX₁₀₉₆).

A compound (L-6) wherein T represents a group represented by T6, R^{3b} represents an iodine atom, and R¹ represents any substituents indicated in [Table L1] to [Table L3](hereinafter, referred to as Compound Class SX₁₀₉₇).

A compound (L-6), wherein T represents a group represented by T7, R^{3b} represents a hydrogen atom, and R¹ represents any substituents indicated in [Table L1] to [Table L3](hereinafter, referred to as Compound Class SX₁₀₉₈).

A compound (L-6) wherein T represents a group represented by T7, R^{3b} represents CF₃, and R¹ represents any substituents indicated in [Table L1] to [Table L3](hereinafter, referred to as Compound Class SX₁₀₉₉).

A compound (L-6) wherein T represents a group represented by T7, R^{3b} represents a chlorine atom, and R¹ represents any substituents indicated in [Table L1] to [Table L3](hereinafter, referred to as Compound Class SX₁₁₀₀).

A compound (L-6) wherein T represents a group represented by T7, R^{3b} represents a bromine atom, and R¹ represents any substituents indicated in [Table L1] to [Table L3](hereinafter, referred to as Compound Class SX₁₁₀₁).

A compound (L-6) wherein T represents a group represented by T7, R^{3b} represents an iodine atom, and R¹ represents any substituents indicated in [Table L1] to [Table L3](hereinafter, referred to as Compound Class SX₁₁₀₂).

A compound (L-6), wherein T represents a group represented by T8, R^{3b} represents a hydrogen atom, and R¹ represents any substituents indicated in [Table L1] to [Table L3](hereinafter, referred to as Compound Class SX₁₁₀₃).

A compound (L-6) wherein T represents a group represented by T8, R^{3b} represents CF₃, and R¹ represents any substituents indicated in [Table L1] to [Table L3](hereinafter, referred to as Compound Class SX₁₁₀₄).

A compound (L-6) wherein T represents a group represented by T8, R^{3b} represents a chlorine atom, and R¹ represents any substituents indicated in [Table L1] to [Table L3](hereinafter, referred to as Compound Class SX₁₁₀₅).

A compound (L-6) wherein T represents a group represented by T8, R^{3b} represents a bromine atom, and R¹ represents any substituents indicated in [Table L1] to [Table L3](hereinafter, referred to as Compound Class SX₁₁₀₆).

A compound (L-6) wherein T represents a group represented by T8, R^{3b} represents an iodine atom, and R¹ represents any substituents indicated in [Table L1] to [Table L3](hereinafter, referred to as Compound Class SX₁₁₀₇).

A compound (L-6), wherein T represents a group represented by T9, R^{3b} represents a hydrogen atom, and R¹ represents any substituents indicated in [Table L1] to [Table L3](hereinafter, referred to as Compound Class SX₁₁₀₈).

A compound (L-6) wherein T represents a group represented by T9, R^{3b} represents CF₃, and R¹ represents any substituents indicated in [Table L1] to [Table L3](hereinafter, referred to as Compound Class SX₁₁₀₉).

A compound (L-6) wherein T represents a group represented by T9, R^{3b} represents a chlorine atom, and R¹ represents any substituents indicated in [Table L1] to [Table L3](hereinafter, referred to as Compound Class SX₁₁₁₁₀).

A compound (L-6) wherein T represents a group represented by T9, R^{3b} represents a bromine atom, and R¹ represents any substituents indicated in [Table L1] to [Table L3](hereinafter, referred to as Compound Class SX₁₁₁₁).

A compound (L-6) wherein T represents a group represented by T9, R^{3b} represents an iodine atom, and R¹ represents any substituents indicated in [Table L1] to [Table L3](hereinafter, referred to as Compound Class SX₁₁₁₂).

A compound (L-6), wherein T represents a group represented by T10, R^{3b} represents a hydrogen atom, and R¹ represents any substituents indicated in [Table L1] to [Table L3](hereinafter, referred to as Compound Class SX₁₁₁₃).

A compound (L-6) wherein T represents a group represented by T10, R^{3b} represents CF₃, and R¹ represents any substituents indicated in [Table L1] to [Table L3](hereinafter, referred to as Compound Class SX₁₁₁₄).

A compound (L-6) wherein T represents a group represented by T10, R^{3b} represents a chlorine atom, and R¹ represents any substituents indicated in [Table L1] to [Table L3](hereinafter, referred to as Compound Class SX₁₁₁₅).

A compound (L-6) wherein T represents a group represented by T10, R^{3b} represents a bromine atom, and R¹ represents any substituents indicated in [Table L1] to [Table L3](hereinafter, referred to as Compound Class SX₁₁₁₆).

A compound (L-6) wherein T represents a group represented by T10, R^{3b} represents an iodine atom, and R¹ represents any substituents indicated in [Table L1] to [Table L3](hereinafter, referred to as Compound Class SX₁₁₁₇).

A compound (L-6), wherein T represents a group represented by T11, R^{3b} represents a hydrogen atom, and R¹ represents any substituents indicated in [Table L1] to [Table L3] (hereinafter, referred to as Compound Class SX₁₁₁₈).

A compound (L-6) wherein T represents a group represented by T11, R^{3b} represents CF₃, and R¹ represents any substituents indicated in [Table L1] to [Table L3](hereinafter, referred to as Compound Class SX₁₁₁₉).

A compound (L-6) wherein T represents a group represented by T11, R^{3b} represents a chlorine atom, and R¹ represents any substituents indicated in [Table L1] to [Table L3] (hereinafter, referred to as Compound Class SX₁₁₂₀).

A compound (L-6) wherein T represents a group represented by T11, R^{3b} represents a bromine atom, and R¹ represents any substituents indicated in [Table L1] to [Table L3] (hereinafter, referred to as Compound Class SX₁₁₂₁).

A compound (L-6) wherein T represents a group represented by T11, R^{3b} represents an iodine atom, and R¹ represents any substituents indicated in [Table L1] to [Table L3] (hereinafter, referred to as Compound Class SX₁₁₂₂).

A compound (L-6), wherein T represents a group represented by T12, R^{3b} represents a hydrogen atom, and R¹ represents any substituents indicated in [Table L1] to [Table L3] (hereinafter, referred to as Compound Class SX₁₁₂₃).

A compound (L-6) wherein T represents a group represented by T12, R^{3b} represents CF₃, and R¹ represents any substituents indicated in [Table L1] to [Table L3] (hereinafter, referred to as Compound Class SX₁₁₂₄).

A compound (L-6) wherein T represents a group represented by T12, R^{3b} represents a chlorine atom, and R¹ represents any substituents indicated in [Table L1] to [Table L3] (hereinafter, referred to as Compound Class SX₁₁₂₅).

A compound (L-6) wherein T represents a group represented by T12, R^{3b} represents a bromine atom, and R¹ represents any substituents indicated in [Table L1] to [Table L3] (hereinafter, referred to as Compound Class SX₁₁₂₆).

A compound (L-6) wherein T represents a group represented by T12, R^{3b} represents an iodine atom, and R¹ represents any substituents indicated in [Table L1] to [Table L3] (hereinafter, referred to as Compound Class SX₁₁₂₇).

A compound (L-6), wherein T represents a group represented by T13, R^{3b} represents a hydrogen atom, and R¹ represents any substituents indicated in [Table L1] to [Table L3] (hereinafter, referred to as Compound Class SX₁₁₂₈).

A compound (L-6) wherein T represents a group represented by T13, R^{3b} represents CF₃, and R¹ represents any substituents indicated in [Table L1] to [Table L3](hereinafter, referred to as Compound Class SX₁₁₂₉).

A compound (L-6) wherein T represents a group represented by T13, R^{3b} represents a chlorine atom, and R¹ represents any substituents indicated in [Table L1] to [Table L3] (hereinafter, referred to as Compound Class SX₁₁₃₀).

A compound (L-6) wherein T represents a group represented by T13, R^{3b} represents a bromine atom, and R¹ represents any substituents indicated in [Table L1] to [Table L3] (hereinafter, referred to as Compound Class SX₁₁₃₁).

A compound (L-6) wherein T represents a group represented by T13, R^{3b} represents an iodine atom, and R¹ represents any substituents indicated in [Table L1] to [Table L3] (hereinafter, referred to as Compound Class SX₁₁₃₂).

A compound (L-6), wherein T represents a group represented by T14, R^{3b} represents a hydrogen atom, and R¹ represents any substituents indicated in [Table L1] to [Table L3] (hereinafter, referred to as Compound Class SX₁₁₃₃).

A compound (L-6) wherein T represents a group represented by T14, R^{3b} represents CF₃, and R¹ represents any substituents indicated in [Table L1] to [Table L3](hereinafter, referred to as Compound Class SX₁₁₃₄).

A compound (L-6) wherein T represents a group represented by T14, R^{3b} represents a chlorine atom, and R¹ represents any substituents indicated in [Table L1] to [Table L3] (hereinafter, referred to as Compound Class SX₁₁₃₅).

A compound (L-6) wherein T represents a group represented by T14, R^{3b} represents a bromine atom, and R¹ represents any substituents indicated in [Table L1] to [Table L3] (hereinafter, referred to as Compound Class SX₁₁₃₆).

A compound (L-6) wherein T represents a group represented by T14, R^{3b} represents an iodine atom, and R¹ represents any substituents indicated in [Table L1] to [Table L3] (hereinafter, referred to as Compound Class SX₁₁₃₇).

A compound (L-6), wherein T represents a group represented by T15, R^{3b} represents a hydrogen atom, and R¹ represents any substituents indicated in [Table L1] to [Table L3] (hereinafter, referred to as Compound Class SX₁₁₃₈).

A compound (L-6) wherein T represents a group represented by T15, R^{3b} represents CF₃, and R¹ represents any substituents indicated in [Table L1] to [Table L3](hereinafter, referred to as Compound Class SX₁₁₃₉).

A compound (L-6) wherein T represents a group represented by T15, R^{3b} represents a chlorine atom, and R¹ represents any substituents indicated in [Table L1] to [Table L3] (hereinafter, referred to as Compound Class SX₁₁₄₀).

A compound (L-6) wherein T represents a group represented by T15, R^{3b} represents a bromine atom, and R¹ represents any substituents indicated in [Table L1] to [Table L3] (hereinafter, referred to as Compound Class SX₁₁₄₁).

A compound (L-6) wherein T represents a group represented by T15, R^{3b} represents an iodine atom, and R¹ represents any substituents indicated in [Table L1] to [Table L3] (hereinafter, referred to as Compound Class SX₁₁₄₂).

A compound (L-6), wherein T represents a group represented by T16, R^{3b} represents a hydrogen atom, and R¹ represents any substituents indicated in [Table L1] to [Table L3] (hereinafter, referred to as Compound Class SX₁₁₄₃).

A compound (L-6) wherein T represents a group represented by T16, R^{3b} represents CF₃, and R¹ represents any substituents indicated in [Table L1] to [Table L3](hereinafter, referred to as Compound Class SX₁₁₄₄).

A compound (L-6) wherein T represents a group represented by T16, R^{3b} represents a chlorine atom, and R¹ represents any substituents indicated in [Table L1] to [Table L3] (hereinafter, referred to as Compound Class SX₁₁₄₅).

A compound (L-6) wherein T represents a group represented by T16, R^{3b} represents a bromine atom, and R¹ represents any substituents indicated in [Table L1] to [Table L3] (hereinafter, referred to as Compound Class SX₁₁₄₆).

A compound (L-6) wherein T represents a group represented by T16, R^{3b} represents an iodine atom, and R¹ represents any substituents indicated in [Table L1] to [Table L3] (hereinafter, referred to as Compound Class SX₁₁₄₇).

A compound (L-6), wherein T represents a group represented by T17, R^{3b} represents a hydrogen atom, and R¹ represents any substituents indicated in [Table L1] to [Table L3] (hereinafter, referred to as Compound Class SX₁₁₄₈).

A compound (L-6) wherein T represents a group represented by T17, R^{3b} represents CF₃, and R¹ represents any substituents indicated in [Table L1] to [Table L3] (hereinafter, referred to as Compound Class SX₁₁₄₉).

A compound (L-6) wherein T represents a group represented by T17, R^{3b} represents a chlorine atom, and R¹ represents any substituents indicated in [Table L1] to [Table L3] (hereinafter, referred to as Compound Class SX₁₁₅₀).

A compound (L-6) wherein T represents a group represented by T17, R^{3b} represents a bromine atom, and R¹ represents any substituents indicated in [Table L1] to [Table L3] (hereinafter, referred to as Compound Class SX₁₁₅₁).

A compound (L-6) wherein T represents a group represented by T17, R^{3b} represents an iodine atom, and R¹ represents any substituents indicated in [Table L1] to [Table L3] (hereinafter, referred to as Compound Class SX₁₁₅₂).

A compound (L-6), wherein T represents a group represented by T18, R^{3b} represents a hydrogen atom, and R¹ represents any substituents indicated in [Table L1] to [Table L3] (hereinafter, referred to as Compound Class SX₁₁₅₃).

A compound (L-6) wherein T represents a group represented by T18, R^{3b} represents CF₃, and R¹ represents any substituents indicated in [Table L1] to [Table L3](hereinafter, referred to as Compound Class SX₁₁₅₄).

A compound (L-6) wherein T represents a group represented by T18, R^{3b} represents a chlorine atom, and R¹ represents any substituents indicated in [Table L1] to [Table L3] (hereinafter, referred to as Compound Class SX₁₁₅₅).

A compound (L-6) wherein T represents a group represented by T18, R^{3b} represents a bromine atom, and R¹ represents any substituents indicated in [Table L1] to [Table L3] (hereinafter, referred to as Compound Class SX₁₁₅₆).

A compound (L-6) wherein T represents a group represented by T18, R³¹ represents an iodine atom, and R¹ represents any substituents indicated in [Table L1] to [Table L3] (hereinafter, referred to as Compound Class SX₁₁₅₇).

A compound (L-6), wherein T represents a group represented by T19, R³¹ represents a hydrogen atom, and R¹ represents any substituents indicated in [Table L1] to [Table L3] (hereinafter, referred to as Compound Class SX₁₁₅₈).

A compound (L-6) wherein T represents a group represented by T19, R^{3b} represents CF₃, and R¹ represents any substituents indicated in [Table L1] to [Table L3](hereinafter, referred to as Compound Class SX₁₁₅₉).

A compound (L-6) wherein T represents a group represented by T19, R³¹ represents a chlorine atom, and R¹ represents any substituents indicated in [Table L1] to [Table L3] (hereinafter, referred to as Compound Class SX₁₁₆₀).

A compound (L-6) wherein T represents a group represented by T19, R³¹ represents a bromine atom, and R¹ represents any substituents indicated in [Table L1] to [Table L3] (hereinafter, referred to as Compound Class SX₁₁₆₁).

A compound (L-6) wherein T represents a group represented by T19, R^{3b} represents an iodine atom, and R¹ represents any substituents indicated in [Table L1] to [Table L3] (hereinafter, referred to as Compound Class SX₁₁₆₂).

A compound (L-6), wherein T represents a group represented by T20, R^{3b} represents a hydrogen atom, and R¹ represents any substituents indicated in [Table L1] to [Table L3] (hereinafter, referred to as Compound Class SX₁₁₆₃).

A compound (L-6) wherein T represents a group represented by T20, R^{3b} represents CF₃, and R¹ represents any substituents indicated in [Table L1] to [Table L3](hereinafter, referred to as Compound Class SX₁₁₆₄).

A compound (L-6) wherein T represents a group represented by T20, R^{3b} represents a chlorine atom, and R¹ represents any substituents indicated in [Table L1] to [Table L3] (hereinafter, referred to as Compound Class SX₁₁₆₅).

A compound (L-6) wherein T represents a group represented by T20, R^{3b} represents a bromine atom, and R¹ represents any substituents indicated in [Table L1] to [Table L3] (hereinafter, referred to as Compound Class SX₁₁₆₆).

A compound (L-6) wherein T represents a group represented by T20, R^{3b} represents an iodine atom, and R¹ represents any substituents indicated in [Table L1] to [Table L3] (hereinafter, referred to as Compound Class SX₁₁₆₇).

A compound (L-6), wherein T represents a group represented by T21, R^{3b} represents a hydrogen atom, and R¹ represents any substituents indicated in [Table L1] to [Table L3] (hereinafter, referred to as Compound Class SX₁₁₆₈).

A compound (L-6) wherein T represents a group represented by T21, R^{3b} represents CF₃, and R¹ represents any substituents indicated in [Table L1] to [Table L3](hereinafter, referred to as Compound Class SX₁₁₆₉).

A compound (L-6) wherein T represents a group represented by T21, R^{3b} represents a chlorine atom, and R¹ represents any substituents indicated in [Table L1] to [Table L3] (hereinafter, referred to as Compound Class SX₁₁₇₀).

A compound (L-6) wherein T represents a group represented by T21, R^{3b} represents a bromine atom, and R¹ represents any substituents indicated in [Table L1] to [Table L3] (hereinafter, referred to as Compound Class SX₁₁₇₁).

A compound (L-6) wherein T represents a group represented by T21, R^{3b} represents an iodine atom, and R¹ represents any substituents indicated in [Table L1] to [Table L3] (hereinafter, referred to as Compound Class SX₁₁₇₂).

A compound (L-6), wherein T represents a group represented by T22, R^{3b} represents a hydrogen atom, and R¹ represents any substituents indicated in [Table L1] to [Table L3] (hereinafter, referred to as Compound Class SX₁₁₇₃).

A compound (L-6) wherein T represents a group represented by T22, R^{3b} represents CF₃, and R¹ represents any substituents indicated in [Table L1] to [Table L3] (hereinafter, referred to as Compound Class SX₁₁₇₄).

A compound (L-6) wherein T represents a group represented by T22, R^{3b} represents a chlorine atom, and R¹ represents any substituents indicated in [Table L1] to [Table L3] (hereinafter, referred to as Compound Class SX₁₁₇₅).

A compound (L-6) wherein T represents a group represented by T22, R^{3b} represents a bromine atom, and R¹ represents any substituents indicated in [Table L1] to [Table L3] (hereinafter, referred to as Compound Class SX₁₁₇₆).

A compound (L-6) wherein T represents a group represented by T22, R^{3b} represents an iodine atom, and R¹ represents any substituents indicated in [Table L1] to [Table L3] (hereinafter, referred to as Compound Class SX₁₁₇₇).

A compound (L-6), wherein T represents a group represented by T23, R^{3b} represents a hydrogen atom, and R¹ represents any substituents indicated in [Table L1] to [Table L3] (hereinafter, referred to as Compound Class SX₁₁₇₈).

A compound (L-6) wherein T represents a group represented by T23, R^{3b} represents CF₃, and R¹ represents any substituents indicated in [Table L1] to [Table L3](hereinafter, referred to as Compound Class SX₁₁₇₉).

A compound (L-6) wherein T represents a group represented by T23, R^{3b} represents a chlorine atom, and R¹ represents any substituents indicated in [Table L1] to [Table L3] (hereinafter, referred to as Compound Class SX₁₁₈₀).

A compound (L-6) wherein T represents a group represented by T23, R^{3b} represents a bromine atom, and R¹ represents any substituents indicated in [Table L1] to [Table L3] (hereinafter, referred to as Compound Class SX₁₁₈₁).

A compound (L-6) wherein T represents a group represented by T23, R^{3b} represents an iodine atom, and R¹ represents any substituents indicated in [Table L1] to [Table L3] (hereinafter, referred to as Compound Class SX₁₁₈₂).

A compound (L-6), wherein T represents a group represented by T24, R^{3b} represents a hydrogen atom, and R¹ represents any substituents indicated in [Table L1] to [Table L3] (hereinafter, referred to as Compound Class SX₁₁₈₃).

A compound (L-6) wherein T represents a group represented by T24, R^{3b} represents CF₃, and R¹ represents any substituents indicated in [Table L1] to [Table L3](hereinafter, referred to as Compound Class SX₁₁₈₄).

A compound (L-6) wherein T represents a group represented by T24, R^{3b} represents a chlorine atom, and R¹ represents any substituents indicated in [Table L1] to [Table L3] (hereinafter, referred to as Compound Class SX₁₁₈₅).

A compound (L-6) wherein T represents a group represented by T24, R^{3b} represents a bromine atom, and R¹ represents any substituents indicated in [Table L1] to [Table L3] (hereinafter, referred to as Compound Class SX₁₁₈₆).

A compound (L-6) wherein T represents a group represented by T24, R^{3b} represents an iodine atom, and R¹ represents any substituents indicated in [Table L1] to [Table L3] (hereinafter, referred to as Compound Class SX₁₁₈₇).

A compound (L-6), wherein T represents a group represented by T25, R^{3b} represents a hydrogen atom, and R¹ represents any substituents indicated in [Table L1] to [Table L3] (hereinafter, referred to as Compound Class SX₁₁₈₈).

A compound (L-6) wherein T represents a group represented by T25, R^{3b} represents CF₃, and R¹ represents any substituents indicated in [Table L1] to [Table L3](hereinafter, referred to as Compound Class SX₁₁₈₉).

A compound (L-6) wherein T represents a group represented by T25, R^{3b} represents a chlorine atom, and R¹ represents any substituents indicated in [Table L1] to [Table L3] (hereinafter, referred to as Compound Class SX₁₁₉₀).

A compound (L-6) wherein T represents a group represented by T25, R^{3b} represents a bromine atom, and R¹ represents any substituents indicated in [Table L1] to [Table L3] (hereinafter, referred to as Compound Class SX₁₁₉₁).

A compound (L-6) wherein T represents a group represented by T25, R^{3b} represents an iodine atom, and R¹ represents any substituents indicated in [Table L1] to [Table L3] (hereinafter, referred to as Compound Class SX₁₁₉₂).

A compound (L-6), wherein T represents a group represented by T26, R^{3b} represents a hydrogen atom, and R¹ represents any substituents indicated in [Table L1] to [Table L3] (hereinafter, referred to as Compound Class SX₁₁₉₃).

A compound (L-6) wherein T represents a group represented by T26, R^{3b} represents CF₃, and R¹ represents any substituents indicated in [Table L1] to [Table L3](hereinafter, referred to as Compound Class SX₁₁₉₄).

A compound (L-6) wherein T represents a group represented by T26, R^{3b} represents a chlorine atom, and R¹ represents any substituents indicated in [Table L1] to [Table L3] (hereinafter, referred to as Compound Class SX₁₁₉₅).

A compound (L-6) wherein T represents a group represented by T26, R^{3b} represents a bromine atom, and R¹ represents any substituents indicated in [Table L1] to [Table L3] (hereinafter, referred to as Compound Class SX₁₁₉₆).

A compound (L-6) wherein T represents a group represented by T26, R^{3b} represents an iodine atom, and R¹ represents any substituents indicated in [Table L1] to [Table L3] (hereinafter, referred to as Compound Class SX₁₁₉₇).

A compound (L-6), wherein T represents a group represented by T27, R^{3b} represents a hydrogen atom, and R¹ represents any substituents indicated in [Table L1] to [Table L3] (hereinafter, referred to as Compound Class SX₁₁₉₈).

A compound (L-6) wherein T represents a group represented by T27, R^{3b} represents CF₃, and R¹ represents any substituents indicated in [Table L1] to [Table L3] (hereinafter, referred to as Compound Class SX₁₁₉₉).

A compound (L-6) wherein T represents a group represented by T27, R^{3b} represents a chlorine atom, and R¹ represents any substituents indicated in [Table L1] to [Table L3] (hereinafter, referred to as Compound Class SX₁₂₀₀).

A compound (L-6) wherein T represents a group represented by T27, R^{3b} represents a bromine atom, and R¹ represents any substituents indicated in [Table L1] to [Table L3] (hereinafter, referred to as Compound Class SX₁₂₀₁).

A compound (L-6) wherein T represents a group represented by T27, R^{3b} represents an iodine atom, and R¹ represents any substituents indicated in [Table L1] to [Table L3] (hereinafter, referred to as Compound Class SX₁₂₀₂).

A compound (L-6), wherein T represents a group represented by T28, R^{3b} represents a hydrogen atom, and R¹ represents any substituents indicated in [Table L1] to [Table L3] (hereinafter, referred to as Compound Class SX₁₂₀₃).

A compound (L-6) wherein T represents a group represented by T28, R^{3b} represents CF₃, and R¹ represents any substituents indicated in [Table L1] to [Table L3](hereinafter, referred to as Compound Class SX₁₂₀₄).

A compound (L-6) wherein T represents a group represented by T28, R^{3b} represents a chlorine atom, and R¹ represents any substituents indicated in [Table L1] to [Table L3] (hereinafter, referred to as Compound Class SX₁₂₀₅).

A compound (L-6) wherein T represents a group represented by T28, R^{3b} represents a bromine atom, and R¹ represents any substituents indicated in [Table L1] to [Table L3] (hereinafter, referred to as Compound Class SX₁₂₀₆).

A compound (L-6) wherein T represents a group represented by T28, R^{3b} represents an iodine atom, and R¹ represents any substituents indicated in [Table L1] to [Table L3] (hereinafter, referred to as Compound Class SX₁₂₀₇).

A compound (L-6), wherein T represents a group represented by T29, R^{3b} represents a hydrogen atom, and R¹ represents any substituents indicated in [Table L1] to [Table L3] (hereinafter, referred to as Compound Class SX₁₂₀₈).

A compound (L-6) wherein T represents a group represented by T29, R^{3b} represents CF₃, and R¹ represents any substituents indicated in [Table L1] to [Table L3](hereinafter, referred to as Compound Class SX₁₂₀₉).

A compound (L-6) wherein T represents a group represented by T29, R^{3b} represents a chlorine atom, and R¹ represents any substituents indicated in [Table L1] to [Table L3] (hereinafter, referred to as Compound Class SX₁₂₁₀).

A compound (L-6) wherein T represents a group represented by T29, R^{3b} represents a bromine atom, and R¹ represents any substituents indicated in [Table L1] to [Table L3] (hereinafter, referred to as Compound Class SX₁₂₁₁).

A compound (L-6) wherein T represents a group represented by T29, R^{3b} represents an iodine atom, and R¹ represents any substituents indicated in [Table L1] to [Table L3] (hereinafter, referred to as Compound Class SX₁₂₁₂).

A compound (L-6), wherein T represents a group represented by T30, R^{3b} represents a hydrogen atom, and R¹ represents any substituents indicated in [Table L1] to [Table L3] (hereinafter, referred to as Compound Class SX₁₂₁₃).

A compound (L-6) wherein T represents a group represented by T30, R^{3b} represents CF₃, and R¹ represents any substituents indicated in [Table L1] to [Table L3](hereinafter, referred to as Compound Class SX₁₂₁₄).

A compound (L-6) wherein T represents a group represented by T30, R^{3b} represents a chlorine atom, and R¹ represents any substituents indicated in [Table L1] to [Table L3] (hereinafter, referred to as Compound Class SX₁₂₁₅).

A compound (L-6) wherein T represents a group represented by T30, R^{3b} represents a bromine atom, and R¹ represents any substituents indicated in [Table L1] to [Table L3] (hereinafter, referred to as Compound Class SX₁₂₁₆).

A compound (L-6) wherein T represents a group represented by T30, R^{3b} represents an iodine atom, and R¹ represents any substituents indicated in [Table L1] to [Table L3] (hereinafter, referred to as Compound Class SX₁₂₁₇).

A compound (L-6), wherein T represents a group represented by T31, R^{3b} represents a hydrogen atom, and R¹ represents any substituents indicated in [Table L1] to [Table L3] (hereinafter, referred to as Compound Class SX₁₂₁₈).

A compound (L-6) wherein T represents a group represented by T31, R^{3b} represents CF₃, and R¹ represents any substituents indicated in [Table L1] to [Table L3](hereinafter, referred to as Compound Class SX₁₂₁₉).

A compound (L-6) wherein T represents a group represented by T31, R^{3b} represents a chlorine atom, and R¹ represents any substituents indicated in [Table L1] to [Table L3] (hereinafter, referred to as Compound Class SX₁₂₂₀).

A compound (L-6) wherein T represents a group represented by T31, R^{3b} represents a bromine atom, and R¹ represents any substituents indicated in [Table L1] to [Table L3] (hereinafter, referred to as Compound Class SX₁₂₂₁).

A compound (L-6) wherein T represents a group represented by T31, R^{3b} represents an iodine atom, and R¹ represents any substituents indicated in [Table L1] to [Table L3] (hereinafter, referred to as Compound Class SX₁₂₂₂).

A compound (L-1) wherein T represents a group represented by T1, R¹ represents CF₃, and R^{3b} represents any substituents indicated in [Table L4] to [Table L12](hereinafter, referred to as Compound Class SX₁₂₂₃).

TABLE L4
F
Br
I
Me
Et
Pr
i-Pr
CHF₂
CH═CH₂
CMe═CH₂
c-Pr
c-Bu
c-Pen
c-Hex
1-F-c-Pr
2,2-F₂-c-Pr
1-CN-c-Pr
1-CN-c-Bu
1-CN-c-Pen
1-CN-c-Hex
CHO
C(O)Me
C(O)c-Pr
C(O)OEt
C(O)NHc-Pr
CH═N—OH
CH═N—OMe
CH═N—OEt
CH═N—OCH₂CF₃
CMe═N—OH
CMe═N—OMe
CMe═N—OEt
CMe═N—OCH₂CF₃
C(NH₂)═N—OCH₂CF₃
NO₂
CN

TABLE L5
Ph
3-F-Ph
4-F-Ph
3-Cl-Ph
4-Cl-Ph
3-CF₃-Ph
4-CF₃-Ph
3-NMe₂-Ph
4-NMe₂-Ph
3-CN-Ph
4-CN-Ph
4-C(O)NMe₂-Ph
4-NHC(O)Me-Ph
3,4-F₂-Ph
3,5-F₂-Ph
2,4-F₂-Ph
3,4,5-F₃-Ph
3,4-Cl₂-Ph
3,5-Cl₂-Ph
3,5-Cl₂-4-F-Ph
OPh
O-2-F-Ph
O-3-CF₃-Ph
O-4-CF₃-Ph
NH₂
NHCH₂CF₃
NHc-Pr
NH(1-CN-c-Pr)
NHOMe
NMe₂
NHC(O)Me
NHC(O)c-Pr
NMeC(O)c-Pr
SEt
S(O)Et
S(O)₂Et

TABLE L6
Py2
4-F-Py2
5-F-Py2
4-Cl-Py2
5-Cl-Py2
4-CF₃-Py2
5-CF₃-Py2
6-CF₃-Py2
3-Me-Py2
4-Me-Py2
5-Me-Py2
6-Me-Py2
4-CN-Py2
5-CN-Py2
5-OCH₂CF₂CF₃-Py2
3,5-F₂-Py2
Py3
6-CF₃-Py3
5-CF₃-Py3
6-F-Py3
6-Cl-Py3
Py4
OPy2
OPy3
OPy4
O-5-CF₃-Py2
O-6-CF₃-Py2
OMe
OEt
OPr
Oi-Pr
Oc-Pr
OCMe₂CN
CMe₂CN
CMeCN₂
CH₂CMe₂CN

TABLE L7

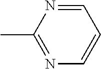

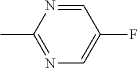

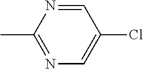

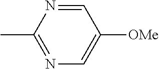

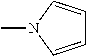

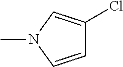

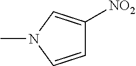

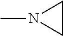

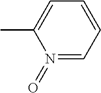

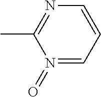

TABLE L8

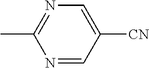

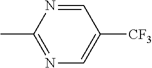

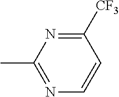

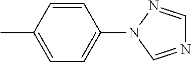

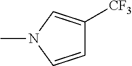

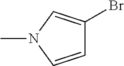

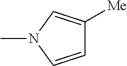

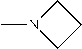

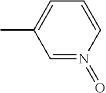

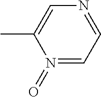

TABLE L9

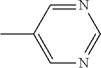

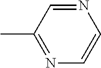

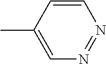

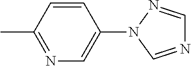

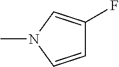

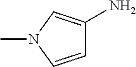

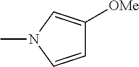

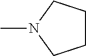

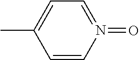

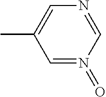

TABLE L10

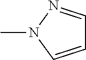

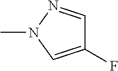

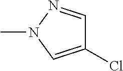

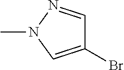

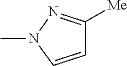

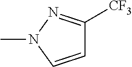

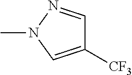

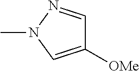

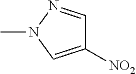

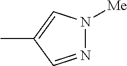

TABLE L11

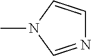

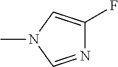

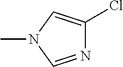

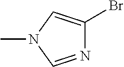

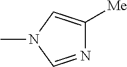

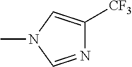

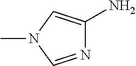

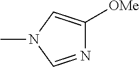

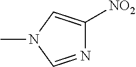

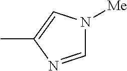

TABLE L12

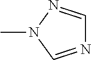

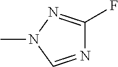

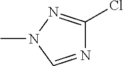

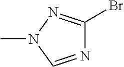

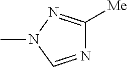

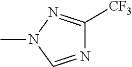

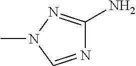

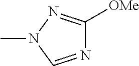

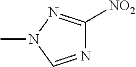

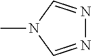

A compound (L-1) wherein T represents a group represented by T1, R¹ represents C₂F₅, and R^{3b} represents any substituents indicated in [Table L4] to [Table L12](hereinafter, referred to as Compound Class SX₁₂₂₄).

A compound (L-1) wherein T represents a group represented by T1, R¹ represents SCF₃, and R^{3b} represents any substituents indicated in [Table L4] to [Table L12](hereinafter, referred to as Compound Class SX₁₂₂₅).

A compound (L-1) wherein T represents a group represented by T1, R¹ represents S(O)CF₃, and R^{3b} represents any substituents indicated in [Table L4] to [Table L12](hereinafter, referred to as Compound Class SX₁₂₂₆).

A compound (L-1) wherein T represents a group represented by T1, R¹ represents S(O)₂CF₃, and R^{3b} represents any substituents indicated in [Table L4] to [Table L12](hereinafter, referred to as Compound Class SX₁₂₂₇).

A compound (L-1) wherein T represents a group represented by T2, R¹ represents CF₃, and R^{3b} represents any substituents indicated in [Table L4] to [Table L12](hereinafter, referred to as Compound Class SX₁₂₂₈).

A compound (L-1) wherein T represents a group represented by T2, R¹ represents C₂F₅, and R^{3b} represents any substituents indicated in [Table L4] to [Table L12](hereinafter, referred to as Compound Class SX₁₂₂₉).

A compound (L-1) wherein T represents a group represented by T2, R¹ represents SCF₃, and R^{3b} represents any substituents indicated in [Table L4] to [Table L12](hereinafter, referred to as Compound Class SX₁₂₃₀).

A compound (L-1) wherein T represents a group represented by T2, R¹ represents S(O)CF₃, and R^{3b} represents any substituents indicated in [Table L4] to [Table L12](hereinafter, referred to as Compound Class SX₁₂₃₁).

A compound (L-1) wherein T represents a group represented by T2, R¹ represents S(O)₂CF₃, and R^{3b} represents any substituents indicated in [Table L4] to [Table L12](hereinafter, referred to as Compound Class SX₁₂₃₂).

A compound (L-1) wherein T represents a group represented by T3, R¹ represents CF₃, and R^{3b} represents any substituents indicated in [Table L4] to [Table L12](hereinafter, referred to as Compound Class SX₁₂₃₃).

A compound (L-1) wherein T represents a group represented by T3, R¹ represents C₂F₅, and R^{3b} represents any substituents indicated in [Table L4] to [Table L12](hereinafter, referred to as Compound Class SX₁₂₃₄).

A compound (L-1) wherein T represents a group represented by T3, R¹ represents SCF₃, and R^{3b} represents any substituents indicated in [Table L4] to [Table L12](hereinafter, referred to as Compound Class SX₁₂₃₅).

A compound (L-1) wherein T represents a group represented by T3, R¹ represents S(O)CF₃, and R^{3b} represents any substituents indicated in [Table L4] to [Table L12](hereinafter, referred to as Compound Class SX₁₂₃₆).

A compound (L-1) wherein T represents a group represented by T3, R¹ represents S(O)₂CF₃, and R^{3b} represents any substituents indicated in [Table L4] to [Table L12](hereinafter, referred to as Compound Class SX₁₂₃₇).

A compound (L-1) wherein T represents a group represented by T6, R¹ represents CF₃, and R^{3b} represents any substituents indicated in [Table L4] to [Table L12](hereinafter, referred to as Compound Class SX₁₂₃₈).

A compound (L-1) wherein T represents a group represented by T6, R¹ represents C₂F₅, and R^{3b} represents any substituents indicated in [Table L4] to [Table L12](hereinafter, referred to as Compound Class SX₁₂₃₉).

A compound (L-1) wherein T represents a group represented by T6, R¹ represents SCF₃, and R^{3b} represents any substituents indicated in [Table L4] to [Table L12](hereinafter, referred to as Compound Class SX₁₂₄₀).

A compound (L-1) wherein T represents a group represented by T6, R¹ represents S(O)CF₃, and R^{3b} represents any substituents indicated in [Table L4] to [Table L12](hereinafter, referred to as Compound Class SX₁₂₄₁).

A compound (L-1) wherein T represents a group represented by T6, R¹ represents S(O)₂CF₃, and R^{3b} represents any substituents indicated in [Table L4] to [Table L12](hereinafter, referred to as Compound Class SX₁₂₄₂).

A compound (L-1) wherein T represents a group represented by T7, R¹ represents CF₃, and R^{3b} represents any substituents indicated in [Table L4] to [Table L12](hereinafter, referred to as Compound Class SX₁₂₄₃).

A compound (L-1) wherein T represents a group represented by T7, R¹ represents C₂F₅, and R^{3b} represents any substituents indicated in [Table L4] to [Table L12](hereinafter, referred to as Compound Class SX₁₂₄₄).

A compound (L-1) wherein T represents a group represented by T7, R¹ represents SCF₃, and R^{3b} represents any substituents indicated in [Table L4] to [Table L12](hereinafter, referred to as Compound Class SX₁₂₄₅).

A compound (L-1) wherein T represents a group represented by T7, R¹ represents S(O)CF₃, and R^{3b} represents any substituents indicated in [Table L4] to [Table L12](hereinafter, referred to as Compound Class SX₁₂₄₆).

A compound (L-1) wherein T represents a group represented by T7, R¹ represents S(O)₂CF₃, and R^{3b} represents any substituents indicated in [Table L4] to [Table L12](hereinafter, referred to as Compound Class SX₁₂₄₇).

A compound (L-1) wherein T represents a group represented by T8, R¹ represents CF₃, and R^{3b} represents any substituents indicated in [Table L4] to [Table L12](hereinafter, referred to as Compound Class SX₁₂₄₈).

A compound (L-1) wherein T represents a group represented by T8, R¹ represents C₂F₅, and R^{3b} represents any substituents indicated in [Table L4] to [Table L12](hereinafter, referred to as Compound Class SX₁₂₄₉).

A compound (L-1) wherein T represents a group represented by T8, R¹ represents SCF₃, and R^{3b} represents any substituents indicated in [Table L4] to [Table L12](hereinafter, referred to as Compound Class SX₁₂₅₀).

A compound (L-1) wherein T represents a group represented by T8, R¹ represents S(O)CF₃, and R^{3b} represents any substituents indicated in [Table L4] to [Table L12](hereinafter, referred to as Compound Class SX₁₂₅₁).

A compound (L-1) wherein T represents a group represented by T8, R¹ represents S(O)₂CF₃, and R^{3b} represents any substituents indicated in [Table L4] to [Table L12](hereinafter, referred to as Compound Class SX₁₂₅₂).

A compound (L-1) wherein T represents a group represented by T11, R¹ represents CF₃, and R^{3b} represents any substituents indicated in [Table L4] to [Table L12](hereinafter, referred to as Compound Class SX₁₂₅₃).

A compound (L-1) wherein T represents a group represented by T11, R¹ represents C₂F₅, and R^{3b} represents any substituents indicated in [Table L4] to [Table L12](hereinafter, referred to as Compound Class SX₁₂₅₄).

A compound (L-1) wherein T represents a group represented by T11, R¹ represents SCF₃, and R^{3b} represents any substituents indicated in [Table L4] to [Table L12](hereinafter, referred to as Compound Class SX₁₂₅₅).

A compound (L-1) wherein T represents a group represented by T11, R¹ represents S(O)CF₃, and R^{3b} represents any substituents indicated in [Table L4] to [Table L12](hereinafter, referred to as Compound Class SX₁₂₅₆).

A compound (L-1) wherein T represents a group represented by T11, R¹ represents S(O)₂CF₃, and R^{3b} represents any substituents indicated in [Table L4] to [Table L12](hereinafter, referred to as Compound Class SX₁₂₅₇).

A compound (L-1) wherein T represents a group represented by T12, R¹ represents CF₃, and R^{3b} represents any substituents indicated in [Table L4] to [Table L12](hereinafter, referred to as Compound Class SX₁₂₅₈).

A compound (L-1) wherein T represents a group represented by T12, R¹ represents C₂F₅, and R^{3b} represents any substituents indicated in [Table L4] to [Table L12](hereinafter, referred to as Compound Class SX₁₂₅₉).

A compound (L-1) wherein T represents a group represented by T12, R¹ represents SCF₃, and R^{3b} represents any substituents indicated in [Table L4] to [Table L12](hereinafter, referred to as Compound Class SX₁₂₆₀).

A compound (L-1) wherein T represents a group represented by T12, R¹ represents S(O)CF₃, and R^{3b} represents any substituents indicated in [Table L4] to [Table L12](hereinafter, referred to as Compound Class SX₁₂₆₁).

A compound (L-1) wherein T represents a group represented by T12, R¹ represents S(O)₂CF₃, and R^{3b} represents any substituents indicated in [Table L4] to [Table L12](hereinafter, referred to as Compound Class SX₁₂₆₂).

A compound (L-1) wherein T represents a group represented by T13, R¹ represents CF₃, and R^{3b} represents any substituents indicated in [Table L4] to [Table L12](hereinafter, referred to as Compound Class SX₁₂₆₃).

A compound (L-1) wherein T represents a group represented by T13, R¹ represents C₂F₅, and R^{3b} represents any substituents indicated in [Table L4] to [Table L12](hereinafter, referred to as Compound Class SX₁₂₆₄).

A compound (L-1) wherein T represents a group represented by T13, R¹ represents SCF₃, and R^{3b} represents any substituents indicated in [Table L4] to [Table L12](hereinafter, referred to as Compound Class SX₁₂₆₅).

A compound (L-1) wherein T represents a group represented by T13, R¹ represents S(O)CF₃, and R^{3b} represents any substituents indicated in [Table L4] to [Table L12](hereinafter, referred to as Compound Class SX₁₂₆₆).

A compound (L-1) wherein T represents a group represented by T13, R¹ represents S(O)₂CF₃, and R^{3b} represents any substituents indicated in [Table L4] to [Table L12](hereinafter, referred to as Compound Class SX₁₂₆₇).

A compound (L-2) wherein T represents a group represented by T1, R¹ represents CF₃, R^{3c} represents a hydrogen atom, and R^{3b} represents any substituents indicated in [Table L4] to [Table L12] (hereinafter, referred to as Compound Class SX₁₂₆₈).

A compound (L-2) wherein T represents a group represented by T1, R¹ represents C₂F₅, R^{3c} represents a hydrogen atom, and R^{3b} represents any substituents indicated in [Table L4] to [Table L12] (hereinafter, referred to as Compound Class SX₁₂₆₉).

A compound (L-2) wherein T represents a group represented by T1, R¹ represents SCF₃, R^{3c} represents a hydrogen atom, and R^{3b} represents any substituents indicated in [Table L4] to [Table L12] (hereinafter, referred to as Compound Class SX₁₂₇₀).

A compound (L-2) wherein T represents a group represented by T1, R¹ represents S(O)CF₃, R^{3c} represents a hydrogen atom, and R^{3b} represents any substituents indicated in [Table L4] to [Table L12] (hereinafter, referred to as Compound Class SX₁₂₇₁).

A compound (L-2) wherein T represents a group represented by T1, R¹ represents S(O)₂CF₃, R^{3c} represents a hydrogen atom, and R^{3b} represents any substituents indicated in [Table L4] to [Table L12] (hereinafter, referred to as Compound Class SX₁₂₇₂).

A compound (L-2) wherein T represents a group represented by T1, R¹ represents CF₃, R^{3b} represents a hydrogen atom, and R^{3c} represents any substituents indicated in [Table L4] to [Table L12] (hereinafter, referred to as Compound Class SX₁₂₇₃).

A compound (L-2) wherein T represents a group represented by T1, R¹ represents C₂F₅, R^{3b} represents a hydrogen atom, and R^{3c} represents any substituents indicated in [Table L4] to [Table L12] (hereinafter, referred to as Compound Class SX₁₂₇₄).

A compound (L-2) wherein T represents a group represented by T1, R¹ represents SCF₃, R^{3b} represents a hydrogen atom, and R^{3c} represents any substituents indicated in [Table L4] to [Table L12] (hereinafter, referred to as Compound Class SX₁₂₇₅).

A compound (L-2) wherein T represents a group represented by T1, R¹ represents S(O)CF₃, R^{3b} represents a hydrogen atom, and R^{3c} represents any substituents indicated in [Table L4] to [Table L12] (hereinafter, referred to as Compound Class SX₁₂₇₆).

A compound (L-2) wherein T represents a group represented by T1, R¹ represents S(O)₂CF₃, R^{3b} represents a hydrogen atom, and R^{3c} represents any substituents indicated in [Table L4] to [Table L12] (hereinafter, referred to as Compound Class SX₁₂₇₇).

A compound (L-2) wherein T represents a group represented by T2, R¹ represents CF₃, R^{3c} represents a hydrogen atom, and R^{3b} represents any substituents indicated in [Table L4] to [Table L12] (hereinafter, referred to as Compound Class SX₁₂₇₈).

A compound (L-2) wherein T represents a group represented by T2, R¹ represents C₂F₅, R^{3c} represents a hydrogen atom, and R^{3b} represents any substituents indicated in [Table L4] to [Table L12] (hereinafter, referred to as Compound Class SX₁₂₇₉).

A compound (L-2) wherein T represents a group represented by T2, R¹ represents SCF₃, R^{3c} represents a hydrogen atom, and R^{3b} represents any substituents indicated in [Table L4] to [Table L12] (hereinafter, referred to as Compound Class SX₁₂₈₀).

A compound (L-2) wherein T represents a group represented by T2, R¹ represents S(O)CF₃, R^{3c} represents a hydrogen atom, and R^{3b} represents any substituents indicated in [Table L4] to [Table L12] (hereinafter, referred to as Compound Class SX₁₂₈₁).

A compound (L-2) wherein T represents a group represented by T2, R¹ represents S(O)₂CF₃, R^{3c} represents a hydrogen atom, and R^{3b} represents any substituents indicated in [Table L4] to [Table L12] (hereinafter, referred to as Compound Class SX₁₂₈₂).

A compound (L-2) wherein T represents a group represented by T2, R¹ represents CF₃, R^{3b} represents a hydrogen atom, and R^{3c} represents any substituents indicated in [Table L4] to [Table L12] (hereinafter, referred to as Compound Class SX₁₂₈₃).

A compound (L-2) wherein T represents a group represented by T2, R¹ represents C₂F₅, R^{3b} represents a hydrogen atom, and R^{3c} represents any substituents indicated in [Table L4] to [Table L12] (hereinafter, referred to as Compound Class SX₁₂₈₄).

A compound (L-2) wherein T represents a group represented by T2, R¹ represents SCF₃, R^{3b} represents a hydrogen atom, and R^{3c} represents any substituents indicated in [Table L4] to [Table L12] (hereinafter, referred to as Compound Class SX₁₂₈₅).

A compound (L-2) wherein T represents a group represented by T2, R¹ represents S(O)CF₃, R^{3b} represents a hydrogen atom, and R^{3c} represents any substituents indicated in [Table L4] to [Table L12] (hereinafter, referred to as Compound Class SX₁₂₈₆).

A compound (L-2) wherein T represents a group represented by T2, R¹ represents S(O)₂CF₃, R^{3b} represents a hydrogen atom, and R^{3c} represents any substituents indicated in [Table L4] to [Table L12] (hereinafter, referred to as Compound Class SX₁₂₈₇).

A compound (L-2) wherein T represents a group represented by T3, R¹ represents CF₃, R^{3c} represents a hydrogen atom, and R^{3b} represents any substituents indicated in [Table L4] to [Table L12] (hereinafter, referred to as Compound Class SX₁₂₈₈).

A compound (L-2) wherein T represents a group represented by T3, R¹ represents C₂F₅, R^{3c} represents a hydrogen atom, and R^{3b} represents any substituents indicated in [Table L4] to [Table L12] (hereinafter, referred to as Compound Class SX₁₂₈₉).

A compound (L-2) wherein T represents a group represented by T3, R¹ represents SCF₃, R^{3c} represents a hydrogen atom, and R^{3b} represents any substituents indicated in [Table L4] to [Table L12] (hereinafter, referred to as Compound Class SX₁₂₉₀).

A compound (L-2) wherein T represents a group represented by T3, R¹ represents S(O)CF₃, R^{3c} represents a hydrogen atom, and R^{3b} represents any substituents indicated in [Table L4] to [Table L12] (hereinafter, referred to as Compound Class SX₁₂₉₁).

A compound (L-2) wherein T represents a group represented by T3, R¹ represents S(O)₂CF₃, R^{3c} represents a hydrogen atom, and R^{3b} represents any substituents indicated in [Table L4] to [Table L12] (hereinafter, referred to as Compound Class SX₁₂₉₂).

A compound (L-2) wherein T represents a group represented by T3, R¹ represents CF₃, R^{3b} represents a hydrogen atom, and R^{3c} represents any substituents indicated in [Table L4] to [Table L12] (hereinafter, referred to as Compound Class SX₁₂₉₃).

A compound (L-2) wherein T represents a group represented by T3, R¹ represents C₂F₅, R^{3b} represents a hydrogen atom, and R^{3c} represents any substituents indicated in [Table L4] to [Table L12] (hereinafter, referred to as Compound Class SX₁₂₉₄).

A compound (L-2) wherein T represents a group represented by T3, R¹ represents SCF₃, R^{3b} represents a hydrogen atom, and R^{3c} represents any substituents indicated in [Table L4] to [Table L12] (hereinafter, referred to as Compound Class SX₁₂₉₅).

A compound (L-2) wherein T represents a group represented by T3, R¹ represents S(O)CF₃, R^{3b} represents a hydrogen atom, and R^{3c} represents any substituents indicated in [Table L4] to [Table L12] (hereinafter, referred to as Compound Class SX₁₂₉₆).

A compound (L-2) wherein T represents a group represented by T3, R¹ represents S(O)₂CF₃, R^{3b} represents a hydrogen atom, and R^{3c} represents any substituents indicated in [Table L4] to [Table L12] (hereinafter, referred to as Compound Class SX₁₂₉₇).

A compound (L-2) wherein T represents a group represented by T6, R¹ represents CF₃, R^{3c} represents a hydrogen atom, and R^{3b} represents any substituents indicated in [Table L4] to [Table L12] (hereinafter, referred to as Compound Class SX₁₂₉₈).

A compound (L-2) wherein T represents a group represented by T6, R¹ represents C₂F₅, R^{3c} represents a hydrogen atom, and R^{3b} represents any substituents indicated in [Table L4] to [Table L12] (hereinafter, referred to as Compound Class SX₁₂₉₉).

A compound (L-2) wherein T represents a group represented by T6, R¹ represents SCF₃, R^{3c} represents a hydrogen atom, and R^{3b} represents any substituents indicated in [Table L4] to [Table L12] (hereinafter, referred to as Compound Class SX₁₃₀₀).

A compound (L-2) wherein T represents a group represented by T6, R¹ represents S(O)CF₃, R^{3c} represents a hydrogen atom, and R^{3b} represents any substituents indicated in [Table L4] to [Table L12] (hereinafter, referred to as Compound Class SX₁₃₀₁).

A compound (L-2) wherein T represents a group represented by T6, R¹ represents S(O)₂CF₃, R^{3c} represents a hydrogen atom, and R^{3b} represents any substituents indicated in [Table L4] to [Table L12] (hereinafter, referred to as Compound Class SX₁₃₀₂).

A compound (L-2) wherein T represents a group represented by T6, R¹ represents CF₃, R^{3b} represents a hydrogen atom, and R^{3c} represents any substituents indicated in [Table L4] to [Table L12] (hereinafter, referred to as Compound Class SX₁₃₀₃).

A compound (L-2) wherein T represents a group represented by T6, R¹ represents C₂F₅, R^{3b} represents a hydrogen atom, and R^{3c} represents any substituents indicated in [Table L4] to [Table L12] (hereinafter, referred to as Compound Class SX₁₃₀₄).

A compound (L-2) wherein T represents a group represented by T6, R¹ represents SCF₃, R^{3b} represents a hydrogen atom, and R^{3c} represents any substituents indicated in [Table L4] to [Table L12] (hereinafter, referred to as Compound Class SX₁₃₀₅).

A compound (L-2) wherein T represents a group represented by T6, R¹ represents S(O)CF₃, R^{3b} represents a hydrogen atom, and R^{3c} represents any substituents indicated in [Table L4] to [Table L12] (hereinafter, referred to as Compound Class SX₁₃₀₆).

A compound (L-2) wherein T represents a group represented by T6, R¹ represents S(O)₂CF₃, R^{3b} represents a hydrogen atom, and R^{3c} represents any substituents indicated in [Table L4] to [Table L12] (hereinafter, referred to as Compound Class SX₁₃₀₇).

A compound (L-2) wherein T represents a group represented by T7, R¹ represents CF₃, R^{3c} represents a hydrogen atom, and R^{3b} represents any substituents indicated in [Table L4] to [Table L12] (hereinafter, referred to as Compound Class SX₁₃₀₈).

A compound (L-2) wherein T represents a group represented by T7, R¹ represents C₂F₅, R^{3c} represents a hydrogen atom, and R^{3b} represents any substituents indicated in [Table L4] to [Table L12] (hereinafter, referred to as Compound Class SX₁₃₀₉).

A compound (L-2) wherein T represents a group represented by T7, R¹ represents SCF₃, R^{3c} represents a hydrogen atom, and R^{3b} represents any substituents indicated in [Table L4] to [Table L12] (hereinafter, referred to as Compound Class SX₁₃₁₀).

A compound (L-2) wherein T represents a group represented by T7, R¹ represents S(O)CF₃, R^{3c} represents a hydrogen atom, and R^{3b} represents any substituents indicated in [Table L4] to [Table L12] (hereinafter, referred to as Compound Class SX₁₃₁₁).

A compound (L-2) wherein T represents a group represented by T7, R¹ represents S(O)₂CF₃, R^{3c} represents a hydrogen atom, and R^{3b} represents any substituents indicated in [Table L4] to [Table L12] (hereinafter, referred to as Compound Class SX₁₃₁₂).

A compound (L-2) wherein T represents a group represented by T7, R¹ represents CF₃, R^{3b} represents a hydrogen atom, and R^{3c} represents any substituents indicated in [Table L4] to [Table L12] (hereinafter, referred to as Compound Class SX₁₃₁₃).

A compound (L-2) wherein T represents a group represented by T7, R¹ represents C₂F₅, R^{3b} represents a hydrogen atom, and R^{3c} represents any substituents indicated in [Table L4] to [Table L12] (hereinafter, referred to as Compound Class SX₁₃₁₄).

A compound (L-2) wherein T represents a group represented by T7, R¹ represents SCF₃, R^{3b} represents a hydrogen atom, and R^{3c} represents any substituents indicated in [Table L4] to [Table L12] (hereinafter, referred to as Compound Class SX₁₃₁₅).

A compound (L-2) wherein T represents a group represented by T7, R¹ represents S(O)CF₃, R^{3b} represents a hydrogen atom, and R^{3c} represents any substituents indicated in [Table L4] to [Table L12] (hereinafter, referred to as Compound Class SX₁₃₁₆).

A compound (L-2) wherein T represents a group represented by T7, R¹ represents S(O)₂CF₃, R^{3b} represents a hydrogen atom, and R^{3c} represents any substituents indicated in [Table L4] to [Table L12] (hereinafter, referred to as Compound Class SX₁₃₁₇).

A compound (L-2) wherein T represents a group represented by T8, R¹ represents CF₃, R^{3c} represents a hydrogen atom, and R^{3b} represents any substituents indicated in [Table L4] to [Table L12] (hereinafter, referred to as Compound Class SX₁₃₁₈).

A compound (L-2) wherein T represents a group represented by T8, R¹ represents C₂F₅, R^{3c} represents a hydrogen atom, and R^{3b} represents any substituents indicated in [Table L4] to [Table L12] (hereinafter, referred to as Compound Class SX₁₃₁₉).

A compound (L-2) wherein T represents a group represented by T8, R¹ represents SCF₃, R^{3c} represents a hydrogen atom, and R^{3b} represents any substituents indicated in [Table L4] to [Table L12] (hereinafter, referred to as Compound Class SX₁₃₂₀).

A compound (L-2) wherein T represents a group represented by T8, R¹ represents S(O)CF₃, R^{3c} represents a hydrogen atom, and R^{3b} represents any substituents indicated in [Table L4] to [Table L12] (hereinafter, referred to as Compound Class SX₁₃₂₁).

A compound (L-2) wherein T represents a group represented by T8, R¹ represents S(O)₂CF₃, R^{3c} represents a hydrogen atom, and R^{3b} represents any substituents indicated in [Table L4] to [Table L12] (hereinafter, referred to as Compound Class SX₁₃₂₂).

A compound (L-2) wherein T represents a group represented by T8, R¹ represents CF₃, R^{3b} represents a hydrogen atom, and R^{3c} represents any substituents indicated in [Table L4] to [Table L12] (hereinafter, referred to as Compound Class SX₁₃₂₃).

A compound (L-2) wherein T represents a group represented by T8, R¹ represents C₂F₅, R^{3b} represents a hydrogen atom, and R^{3c} represents any substituents indicated in [Table L4] to [Table L12] (hereinafter, referred to as Compound Class SX₁₃₂₄).

A compound (L-2) wherein T represents a group represented by T8, R¹ represents SCF₃, R^{3b} represents a hydrogen atom, and R^{3c} represents any substituents indicated in [Table L4] to [Table L12] (hereinafter, referred to as Compound Class SX₁₃₂₅).

A compound (L-2) wherein T represents a group represented by T8, R¹ represents S(O)CF₃, R^{3b} represents a hydrogen atom, and R^{3c} represents any substituents indicated in [Table L4] to [Table L12] (hereinafter, referred to as Compound Class SX₁₃₂₆).

A compound (L-2) wherein T represents a group represented by T8, R¹ represents S(O)₂CF₃, R^{3b} represents a hydrogen atom, and R^{3c} represents any substituents indicated in [Table L4] to [Table L12] (hereinafter, referred to as Compound Class SX₁₃₂₇).

A compound (L-2) wherein T represents a group represented by T11, R¹ represents CF₃, R^{3c} represents a hydrogen atom, and R^{3b} represents any substituents indicated in [Table L4] to [Table L12] (hereinafter, referred to as Compound Class SX₁₃₂₈).

A compound (L-2) wherein T represents a group represented by T11, R¹ represents C₂F₅, R^{3c} represents a hydrogen atom, and R^{3b} represents any substituents indicated in [Table L4] to [Table L12] (hereinafter, referred to as Compound Class SX₁₃₂₉).

A compound (L-2) wherein T represents a group represented by T11, R¹ represents SCF₃, R^{3c} represents a hydrogen atom, and R^{3b} represents any substituents indicated in [Table L4] to [Table L12] (hereinafter, referred to as Compound Class SX₁₃₃₀).

A compound (L-2) wherein T represents a group represented by T11, R¹ represents S(O)CF₃, R^{3c} represents a hydrogen atom, and R^{3b} represents any substituents indicated in [Table L4] to [Table L12] (hereinafter, referred to as Compound Class SX₁₃₃₁).

A compound (L-2) wherein T represents a group represented by T11, R¹ represents S(O)₂CF₃, R^{3c} represents a hydrogen atom, and R^{3b} represents any substituents indicated in [Table L4] to [Table L12] (hereinafter, referred to as Compound Class SX₁₃₃₂).

A compound (L-2) wherein T represents a group represented by T11, R¹ represents CF₃, R^{3b} represents a hydrogen atom, and R^{3c} represents any substituents indicated in [Table L4] to [Table L12] (hereinafter, referred to as Compound Class SX₁₃₃₃).

A compound (L-2) wherein T represents a group represented by T11, R¹ represents C₂F₅, R^{3b} represents a hydrogen atom, and R^{3c} represents any substituents indicated in [Table L4] to [Table L12] (hereinafter, referred to as Compound Class SX₁₃₃₄).

A compound (L-2) wherein T represents a group represented by T11, R¹ represents SCF₃, R^{3b} represents a hydrogen atom, and R^{3c} represents any substituents indicated in [Table L4] to [Table L12] (hereinafter, referred to as Compound Class SX₁₃₃₅).

A compound (L-2) wherein T represents a group represented by T11, R¹ represents S(O)CF₃, R^{3b} represents a hydrogen atom, and R^{3c} represents any substituents indicated in [Table L4] to [Table L12] (hereinafter, referred to as Compound Class SX₁₃₃₆).

A compound (L-2) wherein T represents a group represented by T11, R¹ represents S(O)₂CF₃, R^{3b} represents a hydrogen atom, and R^{3c} represents any substituents indicated in [Table L4] to [Table L12] (hereinafter, referred to as Compound Class SX₁₃₃₇).

A compound (L-2) wherein T represents a group represented by T12, R¹ represents CF₃, R^{3c} represents a hydrogen atom, and R^{3b} represents any substituents indicated in [Table L4] to [Table L12] (hereinafter, referred to as Compound Class SX₁₃₃₈).

A compound (L-2) wherein T represents a group represented by T12, R¹ represents C₂F₅, R^{3c} represents a hydrogen atom, and R^{3b} represents any substituents indicated in [Table L4] to [Table L12] (hereinafter, referred to as Compound Class SX₁₃₃₉).

A compound (L-2) wherein T represents a group represented by T12, R¹ represents SCF₃, R^{3c} represents a hydrogen atom, and R^{3b} represents any substituents indicated in [Table L4] to [Table L12] (hereinafter, referred to as Compound Class SX₁₃₄₀).

A compound (L-2) wherein T represents a group represented by T12, R¹ represents S(O)CF₃, R^{3c} represents a hydrogen atom, and R^{3b} represents any substituents indicated in [Table L4] to [Table L12] (hereinafter, referred to as Compound Class SX₁₃₄₁).

A compound (L-2) wherein T represents a group represented by T12, R¹ represents S(O)₂CF₃, R^{3c} represents a hydrogen atom, and R^{3b} represents any substituents indicated in [Table L4] to [Table L12] (hereinafter, referred to as Compound Class SX₁₃₄₂).

A compound (L-2) wherein T represents a group represented by T12, R¹ represents CF₃, R^{3b} represents a hydrogen atom, and R^{3c} represents any substituents indicated in [Table L4] to [Table L12] (hereinafter, referred to as Compound Class SX₁₃₄₃).

A compound (L-2) wherein T represents a group represented by T12, R¹ represents C₂F₅, R^{3b} represents a hydrogen atom, and R^{3c} represents any substituents indicated in [Table L4] to [Table L12] (hereinafter, referred to as Compound Class SX₁₃₄₄).

A compound (L-2) wherein T represents a group represented by T12, R¹ represents SCF₃, R^{3b} represents a hydrogen atom, and R^{3c} represents any substituents indicated in [Table L4] to [Table L12] (hereinafter, referred to as Compound Class SX₁₃₄₅).

A compound (L-2) wherein T represents a group represented by T12, R¹ represents S(O)CF₃, R^{3b} represents a hydrogen atom, and R^{3c} represents any substituents indicated in [Table L4] to [Table L12] (hereinafter, referred to as Compound Class SX₁₃₄₆).

A compound (L-2) wherein T represents a group represented by T12, R¹ represents S(O)₂CF₃, R^{3b} represents a hydrogen atom, and R^{3c} represents any substituents indicated in [Table L4] to [Table L12] (hereinafter, referred to as Compound Class SX₁₃₄₇).

A compound (L-2) wherein T represents a group represented by T13, R¹ represents CF₃, R^{3c} represents a hydrogen atom, and R^{3b} represents any substituents indicated in [Table L4] to [Table L12] (hereinafter, referred to as Compound Class SX₁₃₄₈).

A compound (L-2) wherein T represents a group represented by T13, R¹ represents C₂F₅, R^{3c} represents a hydrogen atom, and R^{3b} represents any substituents indicated in [Table L4] to [Table L12] (hereinafter, referred to as Compound Class SX₁₃₄₉).

A compound (L-2) wherein T represents a group represented by T13, R¹ represents SCF₃, R^{3c} represents a hydrogen atom, and R^{3b} represents any substituents indicated in [Table L4] to [Table L12] (hereinafter, referred to as Compound Class SX₁₃₅₀).

A compound (L-2) wherein T represents a group represented by T13, R¹ represents S(O)CF₃, R^{3c} represents a hydrogen atom, and R^{3b} represents any substituents indicated in [Table L4] to [Table L12] (hereinafter, referred to as Compound Class SX₁₃₅₁).

A compound (L-2) wherein T represents a group represented by T13, R¹ represents S(O)₂CF₃, R^{3c} represents a hydrogen atom, and R^{3b} represents any substituents indicated in [Table L4] to [Table L12] (hereinafter, referred to as Compound Class SX₁₃₅₂).

A compound (L-2) wherein T represents a group represented by T13, R¹ represents CF₃, R^{3b} represents a hydrogen atom, and R^{3c} represents any substituents indicated in [Table L4] to [Table L12] (hereinafter, referred to as Compound Class SX₁₃₅₃).

A compound (L-2) wherein T represents a group represented by T13, R¹ represents C₂F₅, R^{3b} represents a hydrogen atom, and R^{3c} represents any substituents indicated in [Table L4] to [Table L12] (hereinafter, referred to as Compound Class SX₁₃₅₄).

A compound (L-2) wherein T represents a group represented by T13, R¹ represents SCF₃, R^{3b} represents a hydrogen atom, and R^{3c} represents any substituents indicated in [Table L4] to [Table L12] (hereinafter, referred to as Compound Class SX₁₃₅₅).

A compound (L-2) wherein T represents a group represented by T13, R¹ represents S(O)CF₃, R^{3b} represents a hydrogen atom, and R^{3c} represents any substituents indicated in [Table L4] to [Table L12] (hereinafter, referred to as Compound Class SX₁₃₅₆).

A compound (L-2) wherein T represents a group represented by T13, R¹ represents S(O)₂CF₃, R^{3b} represents a hydrogen atom, and R^{3c} represents any substituents indicated in [Table L4] to [Table L12] (hereinafter, referred to as Compound Class SX₁₃₅₇).

A compound (L-3) wherein T represents a group represented by T1, R¹ represents CF₃, R^{3c} represents a hydrogen atom, and R^{3b} represents any substituents indicated in [Table L4] to [Table L12] (hereinafter, referred to as Compound Class SX₁₃₅₈).

A compound (L-3) wherein T represents a group represented by T1, R¹ represents C₂F₅, R^{3c} represents a hydrogen atom, and R^{3b} represents any substituents indicated in [Table L4] to [Table L12] (hereinafter, referred to as Compound Class SX₁₃₅₉).

A compound (L-3) wherein T represents a group represented by T1, R¹ represents SCF₃, R^{3c} represents a hydrogen atom, and R^{3b} represents any substituents indicated in [Table L4] to [Table L12] (hereinafter, referred to as Compound Class SX₁₃₆₀).

A compound (L-3) wherein T represents a group represented by T1, R¹ represents S(O)CF₃, R^{3c} represents a hydrogen atom, and R^{3b} represents any substituents indicated in [Table L4] to [Table L12] (hereinafter, referred to as Compound Class SX₁₃₆₁).

A compound (L-3) wherein T represents a group represented by T1, R¹ represents S(O)₂CF₃, R^{3c} represents a hydrogen atom, and R^{3b} represents any substituents indicated in [Table L4] to [Table L12] (hereinafter, referred to as Compound Class SX₁₃₆₂).

A compound (L-3) wherein T represents a group represented by T1, R¹ represents CF₃, R³¹ represents a hydrogen atom, and R^{3c} represents any substituents indicated in [Table L4] to [Table L12] (hereinafter, referred to as Compound Class SX₁₃₆₃).

A compound (L-3) wherein T represents a group represented by T1, R¹ represents C₂F₅, R^{3b} represents a hydrogen atom, and R^{3c} represents any substituents indicated in [Table L4] to [Table L12] (hereinafter, referred to as Compound Class SX₁₃₆₄).

A compound (L-3) wherein T represents a group represented by T1, R¹ represents SCF₃, R^{3b} represents a hydrogen atom, and R^{3c} represents any substituents indicated in [Table L4] to [Table L12] (hereinafter, referred to as Compound Class SX₁₃₆₅).

A compound (L-3) wherein T represents a group represented by T1, R¹ represents S(O)CF₃, R^{3b} represents a hydrogen atom, and R^{3c} represents any substituents indicated in [Table L4] to [Table L12] (hereinafter, referred to as Compound Class SX₁₃₆₆).

A compound (L-3) wherein T represents a group represented by T1, R¹ represents S(O)₂CF₃, R^{3b} represents a hydrogen atom, and R^{3c} represents any substituents indicated in [Table L4] to [Table L12] (hereinafter, referred to as Compound Class SX₁₃₆₇).

A compound (L-3) wherein T represents a group represented by T2, R¹ represents CF₃, R^{3c} represents a hydrogen atom, and R^{3b} represents any substituents indicated in [Table L4] to [Table L12] (hereinafter, referred to as Compound Class SX₁₃₆₈).

A compound (L-3) wherein T represents a group represented by T2, R¹ represents C₂F₅, R^{3c} represents a hydrogen atom, and R^{3b} represents any substituents indicated in [Table L4] to [Table L12] (hereinafter, referred to as Compound Class SX₁₃₆₉).

A compound (L-3) wherein T represents a group represented by T2, R¹ represents SCF₃, R^{3c} represents a hydrogen atom, and R^{3b} represents any substituents indicated in [Table L4] to [Table L12] (hereinafter, referred to as Compound Class SX₁₃₇₀).

A compound (L-3) wherein T represents a group represented by T2, R¹ represents S(O)CF₃, R^{3c} represents a hydrogen atom, and R^{3b} represents any substituents indicated in [Table L4] to [Table L12] (hereinafter, referred to as Compound Class SX₁₃₇₁).

A compound (L-3) wherein T represents a group represented by T2, R¹ represents S(O)₂CF₃, R^{3c} represents a hydrogen atom, and R^{3b} represents any substituents indicated in [Table L4] to [Table L12] (hereinafter, referred to as Compound Class SX₁₃₇₂).

A compound (L-3) wherein T represents a group represented by T2, R¹ represents CF₃, R³¹ represents a hydrogen atom, and R^{3c} represents any substituents indicated in [Table L4] to [Table L12] (hereinafter, referred to as Compound Class SX₁₃₇₃).

A compound (L-3) wherein T represents a group represented by T2, R¹ represents C₂F₅, R^{3b} represents a hydrogen atom, and R^{3c} represents any substituents indicated in [Table L4] to [Table L12] (hereinafter, referred to as Compound Class SX₁₃₇₄).

A compound (L-3) wherein T represents a group represented by T2, R¹ represents SCF₃, R^{3b} represents a hydrogen atom, and R^{3c} represents any substituents indicated in [Table L4] to [Table L12] (hereinafter, referred to as Compound Class SX₁₃₇₅).

A compound (L-3) wherein T represents a group represented by T2, R¹ represents S(O)CF₃, R^{3b} represents a hydrogen atom, and R^{3c} represents any substituents indicated in [Table L4] to [Table L12] (hereinafter, referred to as Compound Class SX₁₃₇₆).

A compound (L-3) wherein T represents a group represented by T2, R¹ represents S(O)₂CF₃, R^{3b} represents a hydrogen atom, and R^{3c} represents any substituents indicated in [Table L4] to [Table L12] (hereinafter, referred to as Compound Class SX₁₃₇₇).

A compound (L-3) wherein T represents a group represented by T3, R¹ represents CF₃, R^{3c} represents a hydrogen atom, and R^{3b} represents any substituents indicated in [Table L4] to [Table L12] (hereinafter, referred to as Compound Class SX₁₃₇₈).

A compound (L-3) wherein T represents a group represented by T3, R¹ represents C₂F₅, R^{3c} represents a hydrogen atom, and R^{3b} represents any substituents indicated in [Table L4] to [Table L12] (hereinafter, referred to as Compound Class SX₁₃₇₉).

A compound (L-3) wherein T represents a group represented by T3, R¹ represents SCF₃, R^{3c} represents a hydrogen atom, and R^{3b} represents any substituents indicated in [Table L4] to [Table L12] (hereinafter, referred to as Compound Class SX₁₃₈₀).

A compound (L-3) wherein T represents a group represented by T3, R¹ represents S(O)CF₃, R^{3c} represents a hydrogen atom, and R^{3b} represents any substituents indicated in [Table L4] to [Table L12] (hereinafter, referred to as Compound Class SX₁₃₈₁).

A compound (L-3) wherein T represents a group represented by T3, R¹ represents S(O)₂CF₃, R^{3c} represents a hydrogen atom, and R^{3b} represents any substituents indicated in [Table L4] to [Table L12] (hereinafter, referred to as Compound Class SX₁₃₈₂).

A compound (L-3) wherein T represents a group represented by T3, R¹ represents CF₃, R³¹ represents a hydrogen atom, and R^{3c} represents any substituents indicated in [Table L4] to [Table L12] (hereinafter, referred to as Compound Class SX₁₃₈₃).

A compound (L-3) wherein T represents a group represented by T3, R¹ represents C₂F₅, R^{3b} represents a hydrogen atom, and R^{3c} represents any substituents indicated in [Table L4] to [Table L12] (hereinafter, referred to as Compound Class SX₁₃₈₄).

A compound (L-3) wherein T represents a group represented by T3, R¹ represents SCF₃, R^{3b} represents a hydrogen atom, and R^{3c} represents any substituents indicated in [Table L4] to [Table L12] (hereinafter, referred to as Compound Class SX₁₃₈₅).

A compound (L-3) wherein T represents a group represented by T3, R¹ represents S(O)CF₃, R^{3b} represents a hydrogen atom, and R^{3c} represents any substituents indicated in [Table L4] to [Table L12] (hereinafter, referred to as Compound Class SX₁₃₈₆).

A compound (L-3) wherein T represents a group represented by T3, R¹ represents S(O)₂CF₃, R^{3b} represents a hydrogen atom, and R^{3c} represents any substituents indicated in [Table L4] to [Table L12] (hereinafter, referred to as Compound Class SX₁₃₈₇).

A compound (L-3) wherein T represents a group represented by T6, R¹ represents CF₃, R^{3c} represents a hydrogen atom, and R^{3b} represents any substituents indicated in [Table L4] to [Table L12] (hereinafter, referred to as Compound Class SX₁₃₈₈).

A compound (L-3) wherein T represents a group represented by T6, R¹ represents C₂F₅, R^{3c} represents a hydrogen atom, and R^{3b} represents any substituents indicated in [Table L4] to [Table L12] (hereinafter, referred to as Compound Class SX₁₃₈₉).

A compound (L-3) wherein T represents a group represented by T6, R¹ represents SCF₃, R^{3c} represents a hydrogen atom, and R^{3b} represents any substituents indicated in [Table L4] to [Table L12] (hereinafter, referred to as Compound Class SX₁₃₉₀).

A compound (L-3) wherein T represents a group represented by T6, R¹ represents S(O)CF₃, R^{3c} represents a hydrogen atom, and R^{3b} represents any substituents indicated in [Table L4] to [Table L12] (hereinafter, referred to as Compound Class SX₁₃₉₁).

A compound (L-3) wherein T represents a group represented by T6, R¹ represents S(O)₂CF₃, R^{3c} represents a hydrogen atom, and R^{3b} represents any substituents indicated in [Table L4] to [Table L12] (hereinafter, referred to as Compound Class SX₁₃₉₂).

A compound (L-3) wherein T represents a group represented by T6, R¹ represents CF₃, R³¹ represents a hydrogen atom, and R^{3c} represents any substituents indicated in [Table L4] to [Table L12] (hereinafter, referred to as Compound Class SX₁₃₉₃).

A compound (L-3) wherein T represents a group represented by T6, R¹ represents C₂F₅, R^{3b} represents a hydrogen atom, and R^{3c} represents any substituents indicated in [Table L4] to [Table L12] (hereinafter, referred to as Compound Class SX₁₃₉₄).

A compound (L-3) wherein T represents a group represented by T6, R¹ represents SCF₃, R^{3b} represents a hydrogen atom, and R^{3c} represents any substituents indicated in [Table L4] to [Table L12] (hereinafter, referred to as Compound Class SX₁₃₉₅).

A compound (L-3) wherein T represents a group represented by T6, R¹ represents S(O)CF₃, R^{3b} represents a hydrogen atom, and R^{3c} represents any substituents indicated in [Table L4] to [Table L12] (hereinafter, referred to as Compound Class SX₁₃₉₆).

A compound (L-3) wherein T represents a group represented by T6, R¹ represents S(O)₂CF₃, R^{3b} represents a hydrogen atom, and R^{3c} represents any substituents indicated in [Table L4] to [Table L12] (hereinafter, referred to as Compound Class SX₁₃₉₇).

A compound (L-3) wherein T represents a group represented by T7, R¹ represents CF₃, R^{3c} represents a hydrogen atom, and R^{3b} represents any substituents indicated in [Table L4] to [Table L12] (hereinafter, referred to as Compound Class SX₁₃₉₈).

A compound (L-3) wherein T represents a group represented by T7, R¹ represents C₂F₅, R^{3c} represents a hydrogen atom, and R^{3b} represents any substituents indicated in [Table L4] to [Table L12] (hereinafter, referred to as Compound Class SX₁₃₉₉).

A compound (L-3) wherein T represents a group represented by T7, R¹ represents SCF₃, R^{3c} represents a hydrogen atom, and R^{3b} represents any substituents indicated in [Table L4] to [Table L12] (hereinafter, referred to as Compound Class SX₁₄₀₀).

A compound (L-3) wherein T represents a group represented by T7, R¹ represents S(O)CF₃, R^{3c} represents a hydrogen atom, and R^{3b} represents any substituents indicated in [Table L4] to [Table L12] (hereinafter, referred to as Compound Class SX₁₄₀₁).

A compound (L-3) wherein T represents a group represented by T7, R¹ represents S(O)₂CF₃, R^{3c} represents a hydrogen atom, and R^{3b} represents any substituents indicated in [Table L4] to [Table L12] (hereinafter, referred to as Compound Class SX₁₄₀₂).

A compound (L-3) wherein T represents a group represented by T7, R¹ represents CF₃, R³¹ represents a hydrogen atom, and R^{3c} represents any substituents indicated in [Table L4] to [Table L12] (hereinafter, referred to as Compound Class SX₁₄₀₃).

A compound (L-3) wherein T represents a group represented by T7, R¹ represents C₂F₅, R^{3b} represents a hydrogen atom, and R^{3c} represents any substituents indicated in [Table L4] to [Table L12] (hereinafter, referred to as Compound Class SX₁₄₀₄).

A compound (L-3) wherein T represents a group represented by T7, R¹ represents SCF₃, R^{3b} represents a hydrogen atom, and R^{3c} represents any substituents indicated in [Table L4] to [Table L12] (hereinafter, referred to as Compound Class SX₁₄₀₅).

A compound (L-3) wherein T represents a group represented by T7, R¹ represents S(O)CF₃, R^{3b} represents a hydrogen atom, and R^{3c} represents any substituents indicated in [Table L4] to [Table L12] (hereinafter, referred to as Compound Class SX₁₄₀₆).

A compound (L-3) wherein T represents a group represented by T7, R¹ represents S(O)₂CF₃, R^{3b} represents a hydrogen atom, and R^{3c} represents any substituents indicated in [Table L4] to [Table L12] (hereinafter, referred to as Compound Class SX₁₄₀₇).

A compound (L-3) wherein T represents a group represented by T8, R¹ represents CF₃, R^{3c} represents a hydrogen atom, and R^{3b} represents any substituents indicated in [Table L4] to [Table L12] (hereinafter, referred to as Compound Class SX₁₄₀₈).

A compound (L-3) wherein T represents a group represented by T8, R¹ represents C₂F₅, R^{3c} represents a hydrogen atom, and R^{3b} represents any substituents indicated in [Table L4] to [Table L12] (hereinafter, referred to as Compound Class SX₁₄₀₉).

A compound (L-3) wherein T represents a group represented by T8, R¹ represents SCF₃, R^{3c} represents a hydrogen atom, and R^{3b} represents any substituents indicated in [Table L4] to [Table L12] (hereinafter, referred to as Compound Class SX₁₄₁₀).

A compound (L-3) wherein T represents a group represented by T8, R¹ represents S(O)CF₃, R^{3c} represents a hydrogen atom, and R^{3b} represents any substituents indicated in [Table L4] to [Table L12] (hereinafter, referred to as Compound Class SX₁₄₁₁).

A compound (L-3) wherein T represents a group represented by T8, R¹ represents S(O)₂CF₃, R^{3c} represents a hydrogen atom, and R^{3b} represents any substituents indicated in [Table L4] to [Table L12] (hereinafter, referred to as Compound Class SX₁₄₁₂).

A compound (L-3) wherein T represents a group represented by T8, R¹ represents CF₃, R³¹ represents a hydrogen atom, and R^{3c} represents any substituents indicated in [Table L4] to [Table L12] (hereinafter, referred to as Compound Class SX₁₄₁₃).

A compound (L-3) wherein T represents a group represented by T8, R¹ represents C₂F₅, R^{3b} represents a hydrogen atom, and R^{3c} represents any substituents indicated in [Table L4] to [Table L12] (hereinafter, referred to as Compound Class SX₁₄₁₄).

A compound (L-3) wherein T represents a group represented by T8, R¹ represents SCF₃, R^{3b} represents a hydrogen atom, and R^{3c} represents any substituents indicated in [Table L4] to [Table L12] (hereinafter, referred to as Compound Class SX₁₄₁₅).

A compound (L-3) wherein T represents a group represented by T8, R¹ represents S(O)CF₃, R^{3b} represents a hydrogen atom, and R^{3c} represents any substituents indicated in [Table L4] to [Table L12] (hereinafter, referred to as Compound Class SX₁₄₁₆).

A compound (L-3) wherein T represents a group represented by T8, R¹ represents S(O)₂CF₃, R^{3b} represents a hydrogen atom, and R^{3c} represents any substituents indicated in [Table L4] to [Table L12] (hereinafter, referred to as Compound Class SX₁₄₁₇).

A compound (L-3) wherein T represents a group represented by T11, R¹ represents CF₃, R^{3c} represents a hydrogen atom, and R^{3b} represents any substituents indicated in [Table L4] to [Table L12] (hereinafter, referred to as Compound Class SX₁₄₁₈).

A compound (L-3) wherein T represents a group represented by T11, R¹ represents C₂F₅, R^{3c} represents a hydrogen atom, and R^{3b} represents any substituents indicated in [Table L4] to [Table L12] (hereinafter, referred to as Compound Class SX₁₄₁₉).

A compound (L-3) wherein T represents a group represented by T11, R¹ represents SCF₃, R^{3c} represents a hydrogen atom, and R^{3b} represents any substituents indicated in [Table L4] to [Table L12] (hereinafter, referred to as Compound Class SX₁₄₂₀).

A compound (L-3) wherein T represents a group represented by T11, R¹ represents S(O)CF₃, R^{3c} represents a hydrogen atom, and R^{3b} represents any substituents indicated in [Table L4] to [Table L12] (hereinafter, referred to as Compound Class SX₁₄₂₁).

A compound (L-3) wherein T represents a group represented by T11, R¹ represents S(O)₂CF₃, R^{3c} represents a hydrogen atom, and R^{3b} represents any substituents indicated in [Table L4] to [Table L12] (hereinafter, referred to as Compound Class SX₁₄₂₂).

A compound (L-3) wherein T represents a group represented by T11, R¹ represents CF₃, R³¹ represents a hydrogen atom, and R^{3c} represents any substituents indicated in [Table L4] to [Table L12] (hereinafter, referred to as Compound Class SX₁₄₂₃).

A compound (L-3) wherein T represents a group represented by T11, R¹ represents C₂F₅, R^{3b} represents a hydrogen atom, and R^{3c} represents any substituents indicated in [Table L4] to [Table L12] (hereinafter, referred to as Compound Class SX₁₄₂₄).

A compound (L-3) wherein T represents a group represented by T11, R¹ represents SCF₃, R^{3b} represents a hydrogen atom, and R^{3c} represents any substituents indicated in [Table L4] to [Table L12] (hereinafter, referred to as Compound Class SX₁₄₂₅).

A compound (L-3) wherein T represents a group represented by T11, R¹ represents S(O)CF₃, R^{3b} represents a hydrogen atom, and R^{3c} represents any substituents indicated in [Table L4] to [Table L12] (hereinafter, referred to as Compound Class SX₁₄₂₆).

A compound (L-3) wherein T represents a group represented by T11, R¹ represents S(O)₂CF₃, R^{3b} represents a hydrogen atom, and R^{3c} represents any substituents indicated in [Table L4] to [Table L12] (hereinafter, referred to as Compound Class SX₁₄₂₇).

A compound (L-3) wherein T represents a group represented by T12, R¹ represents CF₃, R^{3c} represents a hydrogen atom, and R^{3b} represents any substituents indicated in [Table L4] to [Table L12] (hereinafter, referred to as Compound Class SX₁₄₂₈).

A compound (L-3) wherein T represents a group represented by T12, R¹ represents C₂F₅, R^{3c} represents a hydrogen atom, and R^{3b} represents any substituents indicated in [Table L4] to [Table L12] (hereinafter, referred to as Compound Class SX₁₄₂₉).

A compound (L-3) wherein T represents a group represented by T12, R¹ represents SCF₃, R^{3c} represents a hydrogen atom, and R^{3b} represents any substituents indicated in [Table L4] to [Table L12] (hereinafter, referred to as Compound Class SX₁₄₃₀).

A compound (L-3) wherein T represents a group represented by T12, R¹ represents S(O)CF₃, R^{3c} represents a hydrogen atom, and R^{3b} represents any substituents indicated in [Table L4] to [Table L12] (hereinafter, referred to as Compound Class SX₁₄₃₁).

A compound (L-3) wherein T represents a group represented by T12, R¹ represents S(O)₂CF₃, R^{3c} represents a hydrogen atom, and R^{3b} represents any substituents indicated in [Table L4] to [Table L12] (hereinafter, referred to as Compound Class SX₁₄₃₂).

A compound (L-3) wherein T represents a group represented by T12, R¹ represents CF₃, R³¹ represents a hydrogen atom, and R^{3c} represents any substituents indicated in [Table L4] to [Table L12] (hereinafter, referred to as Compound Class SX₁₄₃₃).

A compound (L-3) wherein T represents a group represented by T12, R¹ represents C₂F₅, R^{3b} represents a hydrogen atom, and R^{3c} represents any substituents indicated in [Table L4] to [Table L12] (hereinafter, referred to as Compound Class SX₁₄₃₄).

A compound (L-3) wherein T represents a group represented by T12, R¹ represents SCF₃, R^{3b} represents a hydrogen atom, and R^{3c} represents any substituents indicated in [Table L4] to [Table L12] (hereinafter, referred to as Compound Class SX₁₄₃₅).

A compound (L-3) wherein T represents a group represented by T12, R¹ represents S(O)CF₃, R^{3b} represents a hydrogen atom, and R^{3c} represents any substituents indicated in [Table L4] to [Table L12] (hereinafter, referred to as Compound Class SX₁₄₃₆).

A compound (L-3) wherein T represents a group represented by T12, R¹ represents S(O)₂CF₃, R^{3b} represents a hydrogen atom, and R^{3c} represents any substituents indicated in [Table L4] to [Table L12] (hereinafter, referred to as Compound Class SX₁₄₃₇).

A compound (L-3) wherein T represents a group represented by T13, R¹ represents CF₃, R^{3c} represents a hydrogen atom, and R^{3b} represents any substituents indicated in [Table L4] to [Table L12] (hereinafter, referred to as Compound Class SX₁₄₃₈).

A compound (L-3) wherein T represents a group represented by T13, R¹ represents C₂F₅, R^{3c} represents a hydrogen atom, and R^{3b} represents any substituents indicated in [Table L4] to [Table L12] (hereinafter, referred to as Compound Class SX₁₄₃₉).

A compound (L-3) wherein T represents a group represented by T13, R¹ represents SCF₃, R^{3c} represents a hydrogen atom, and R^{3b} represents any substituents indicated in [Table L4] to [Table L12] (hereinafter, referred to as Compound Class SX₁₄₄₀).

A compound (L-3) wherein T represents a group represented by T13, R¹ represents S(O)CF₃, R^{3c} represents a hydrogen atom, and R^{3b} represents any substituents indicated in [Table L4] to [Table L12] (hereinafter, referred to as Compound Class SX₁₄₄₁).

A compound (L-3) wherein T represents a group represented by T13, R¹ represents S(O)₂CF₃, R^{3c} represents a hydrogen atom, and R^{3b} represents any substituents indicated in [Table L4] to [Table L12] (hereinafter, referred to as Compound Class SX₁₄₄₂).

A compound (L-3) wherein T represents a group represented by T13, R¹ represents CF₃, R³¹ represents a hydrogen atom, and R^{3c} represents any substituents indicated in [Table L4] to [Table L12] (hereinafter, referred to as Compound Class SX₁₄₄₃).

A compound (L-3) wherein T represents a group represented by T13, R¹ represents C₂F₅, R^{3b} represents a hydrogen atom, and R^{3c} represents any substituents indicated in [Table L4] to [Table L12] (hereinafter, referred to as Compound Class SX₁₄₄₄).

A compound (L-3) wherein T represents a group represented by T13, R¹ represents SCF₃, R^{3b} represents a hydrogen atom, and R^{3c} represents any substituents indicated in [Table L4] to [Table L12] (hereinafter, referred to as Compound Class SX₁₄₄₅).

A compound (L-3) wherein T represents a group represented by T13, R¹ represents S(O)CF₃, R^{3b} represents a hydrogen atom, and R^{3c} represents any substituents indicated in [Table L4] to [Table L12] (hereinafter, referred to as Compound Class SX₁₄₄₆).

A compound (L-3) wherein T represents a group represented by T13, R¹ represents S(O)₂CF₃, R^{3b} represents a hydrogen atom, and R^{3c} represents any substituents indicated in [Table L4] to [Table L12] (hereinafter, referred to as Compound Class SX₁₄₄₇).

A compound (L-4) wherein T represents a group represented by T1, R¹ represents CF₃, R^{3c} represents a hydrogen atom, and R^{3b} represents any substituents indicated in [Table L4] to [Table L12] (hereinafter, referred to as Compound Class SX₁₄₄₈).

A compound (L-4) wherein T represents a group represented by T1, R¹ represents C₂F₅, R^{3c} represents a hydrogen atom, and R^{3b} represents any substituents indicated in [Table L4] to [Table L12] (hereinafter, referred to as Compound Class SX₁₄₄₉).

A compound (L-4) wherein T represents a group represented by T1, R¹ represents SCF₃, R^{3c} represents a hydrogen atom, and R^{3b} represents any substituents indicated in [Table L4] to [Table L12] (hereinafter, referred to as Compound Class SX₁₄₅₀).

A compound (L-4) wherein T represents a group represented by T1, R¹ represents S(O)CF₃, R^{3c} represents a hydrogen atom, and R^{3b} represents any substituents indicated in [Table L4] to [Table L12] (hereinafter, referred to as Compound Class SX₁₄₅₁).

A compound (L-4) wherein T represents a group represented by T1, R¹ represents S(O)₂CF₃, R^{3c} represents a hydrogen atom, and R^{3b} represents any substituents indicated in [Table L4] to [Table L12] (hereinafter, referred to as Compound Class SX₁₄₅₂).

A compound (L-4) wherein T represents a group represented by T1, R¹ represents CF₃, R^{3b} represents a hydrogen atom, and R^{3c} represents any substituents indicated in [Table L4] to [Table L12] (hereinafter, referred to as Compound Class SX₁₄₅₃).

A compound (L-4) wherein T represents a group represented by T1, R¹ represents C₂F₅, R^{3b} represents a hydrogen atom, and R^{3c} represents any substituents indicated in [Table L4] to [Table L12] (hereinafter, referred to as Compound Class SX₁₄₅₄).

A compound (L-4) wherein T represents a group represented by T1, R¹ represents SCF₃, R^{3b} represents a hydrogen atom, and R^{3c} represents any substituents indicated in [Table L4] to [Table L12] (hereinafter, referred to as Compound Class SX₁₄₅₅).

A compound (L-4) wherein T represents a group represented by T1, R¹ represents S(O)CF₃, R^{3b} represents a hydrogen atom, and R^{3c} represents any substituents indicated in [Table L4] to [Table L12] (hereinafter, referred to as Compound Class SX₁₄₅₆).

A compound (L-4) wherein T represents a group represented by T1, R¹ represents S(O)₂CF₃, R^{3b} represents a hydrogen atom, and R^{3c} represents any substituents indicated in [Table L4] to [Table L12] (hereinafter, referred to as Compound Class SX₁₄₅₇).

A compound (L-4) wherein T represents a group represented by T2, R¹ represents CF₃, R^{3c} represents a hydrogen atom, and R^{3b} represents any substituents indicated in [Table L4] to [Table L12] (hereinafter, referred to as Compound Class SX₁₄₅₈).

A compound (L-4) wherein T represents a group represented by T2, R¹ represents C₂F₅, R^{3c} represents a hydrogen atom, and R^{3b} represents any substituents indicated in [Table L4] to [Table L12] (hereinafter, referred to as Compound Class SX₁₄₅₉).

A compound (L-4) wherein T represents a group represented by T2, R¹ represents SCF₃, R^{3c} represents a hydrogen atom, and R^{3b} represents any substituents indicated in [Table L4] to [Table L12] (hereinafter, referred to as Compound Class SX₁₄₆₀).

A compound (L-4) wherein T represents a group represented by T2, R¹ represents S(O)CF₃, R^{3c} represents a hydrogen atom, and R^{3b} represents any substituents indicated in [Table L4] to [Table L12] (hereinafter, referred to as Compound Class SX₁₄₆₁).

A compound (L-4) wherein T represents a group represented by T2, R¹ represents S(O)₂CF₃, R^{3c} represents a hydrogen atom, and R^{3b} represents any substituents indicated in [Table L4] to [Table L12] (hereinafter, referred to as Compound Class SX₁₄₆₂).

A compound (L-4) wherein T represents a group represented by T2, R¹ represents CF₃, R^{3b} represents a hydrogen atom, and R^{3c} represents any substituents indicated in [Table L4] to [Table L12] (hereinafter, referred to as Compound Class SX₁₄₆₃).

A compound (L-4) wherein T represents a group represented by T2, R¹ represents C₂F₅, R^{3b} represents a hydrogen atom, and R^{3c} represents any substituents indicated in [Table L4] to [Table L12] (hereinafter, referred to as Compound Class SX₁₄₆₄).

A compound (L-4) wherein T represents a group represented by T2, R¹ represents SCF₃, R^{3b} represents a hydrogen atom, and R^{3c} represents any substituents indicated in [Table L4] to [Table L12] (hereinafter, referred to as Compound Class SX₁₄₆₅).

A compound (L-4) wherein T represents a group represented by T2, R¹ represents S(O)CF₃, R^{3b} represents a hydrogen atom, and R^{3c} represents any substituents indicated in [Table L4] to [Table L12] (hereinafter, referred to as Compound Class SX₁₄₆₆).

A compound (L-4) wherein T represents a group represented by T2, R¹ represents S(O)₂CF₃, R^{3b} represents a hydrogen atom, and R^{3c} represents any substituents indicated in [Table L4] to [Table L12] (hereinafter, referred to as Compound Class SX₁₄₆₇).

A compound (L-4) wherein T represents a group represented by T3, R¹ represents CF₃, R^{3c} represents a hydrogen atom, and R^{3b} represents any substituents indicated in [Table L4] to [Table L12] (hereinafter, referred to as Compound Class SX₁₄₆₈).

A compound (L-4) wherein T represents a group represented by T3, R¹ represents C₂F₅, R^{3c} represents a hydrogen atom, and R^{3b} represents any substituents indicated in [Table L4] to [Table L12] (hereinafter, referred to as Compound Class SX₁₄₆₉).

A compound (L-4) wherein T represents a group represented by T3, R¹ represents SCF₃, R^{3c} represents a hydrogen atom, and R^{3b} represents any substituents indicated in [Table L4] to [Table L12] (hereinafter, referred to as Compound Class SX₁₄₇₀).

A compound (L-4) wherein T represents a group represented by T3, R¹ represents S(O)CF₃, R^{3c} represents a hydrogen atom, and R^{3b} represents any substituents indicated in [Table L4] to [Table L12] (hereinafter, referred to as Compound Class SX₁₄₇₁).

A compound (L-4) wherein T represents a group represented by T3, R¹ represents S(O)₂CF₃, R^{3c} represents a hydrogen atom, and R^{3b} represents any substituents indicated in [Table L4] to [Table L12] (hereinafter, referred to as Compound Class SX₁₄₇₂).

A compound (L-4) wherein T represents a group represented by T3, R¹ represents CF₃, R^{3b} represents a hydrogen atom, and R^{3c} represents any substituents indicated in [Table L4] to [Table L12] (hereinafter, referred to as Compound Class SX₁₄₇₃).

A compound (L-4) wherein T represents a group represented by T3, R¹ represents C₂F₅, R^{3b} represents a hydrogen atom, and R^{3c} represents any substituents indicated in [Table L4] to [Table L12] (hereinafter, referred to as Compound Class SX₁₄₇₄).

A compound (L-4) wherein T represents a group represented by T3, R¹ represents SCF₃, R^{3b} represents a hydrogen atom, and R^{3c} represents any substituents indicated in [Table L4] to [Table L12] (hereinafter, referred to as Compound Class SX₁₄₇₅).

A compound (L-4) wherein T represents a group represented by T3, R¹ represents S(O)CF₃, R^{3b} represents a hydrogen atom, and R^{3c} represents any substituents indicated in [Table L4] to [Table L12] (hereinafter, referred to as Compound Class SX₁₄₇₆).

A compound (L-4) wherein T represents a group represented by T3, R¹ represents S(O)₂CF₃, R^{3b} represents a hydrogen atom, and R^{3c} represents any substituents indicated in [Table L4] to [Table L12] (hereinafter, referred to as Compound Class SX₁₄₇₇).

A compound (L-4) wherein T represents a group represented by T6, R¹ represents CF₃, R^{3c} represents a hydrogen atom, and R^{3b} represents any substituents indicated in [Table L4] to [Table L12] (hereinafter, referred to as Compound Class SX₁₄₇₈).

A compound (L-4) wherein T represents a group represented by T6, R¹ represents C₂F₅, R^{3c} represents a hydrogen atom, and R^{3b} represents any substituents indicated in [Table L4] to [Table L12] (hereinafter, referred to as Compound Class SX₁₄₇₉).

A compound (L-4) wherein T represents a group represented by T6, R¹ represents SCF₃, R^{3c} represents a hydrogen atom, and R^{3b} represents any substituents indicated in [Table L4] to [Table L12] (hereinafter, referred to as Compound Class SX₁₄₈₀).

A compound (L-4) wherein T represents a group represented by T6, R¹ represents S(O)CF₃, R^{3c} represents a hydrogen atom, and R^{3b} represents any substituents indicated in [Table L4] to [Table L12] (hereinafter, referred to as Compound Class SX₁₄₈₁).

A compound (L-4) wherein T represents a group represented by T6, R¹ represents S(O)₂CF₃, R^{3c} represents a hydrogen atom, and R^{3b} represents any substituents indicated in [Table L4] to [Table L12] (hereinafter, referred to as Compound Class SX₁₄₈₂).

A compound (L-4) wherein T represents a group represented by T6, R¹ represents CF₃, R^{3b} represents a hydrogen atom, and R^{3c} represents any substituents indicated in [Table L4] to [Table L12] (hereinafter, referred to as Compound Class SX₁₄₈₃).

A compound (L-4) wherein T represents a group represented by T6, R¹ represents C₂F₅, R^{3b} represents a hydrogen atom, and R^{3c} represents any substituents indicated in [Table L4] to [Table L12] (hereinafter, referred to as Compound Class SX₁₄₈₄).

A compound (L-4) wherein T represents a group represented by T6, R¹ represents SCF₃, R^{3b} represents a hydrogen atom, and R^{3c} represents any substituents indicated in [Table L4] to [Table L12] (hereinafter, referred to as Compound Class SX₁₄₈₅).

A compound (L-4) wherein T represents a group represented by T6, R¹ represents S(O)CF₃, R^{3b} represents a hydrogen atom, and R^{3c} represents any substituents indicated in [Table L4] to [Table L12] (hereinafter, referred to as Compound Class SX₁₄₈₆).

A compound (L-4) wherein T represents a group represented by T6, R¹ represents S(O)₂CF₃, R^{3b} represents a hydrogen atom, and R^{3c} represents any substituents indicated in [Table L4] to [Table L12] (hereinafter, referred to as Compound Class SX₁₄₈₇).

A compound (L-4) wherein T represents a group represented by T7, R¹ represents CF₃, R^{3c} represents a hydrogen atom, and R^{3b} represents any substituents indicated in [Table L4] to [Table L12] (hereinafter, referred to as Compound Class SX₁₄₈₈).

A compound (L-4) wherein T represents a group represented by T7, R¹ represents C₂F₅, R^{3c} represents a hydrogen atom, and R^{3b} represents any substituents indicated in [Table L4] to [Table L12] (hereinafter, referred to as Compound Class SX₁₄₈₉).

A compound (L-4) wherein T represents a group represented by T7, R¹ represents SCF₃, R^{3c} represents a hydrogen atom, and R^{3b} represents any substituents indicated in [Table L4] to [Table L12] (hereinafter, referred to as Compound Class SX₁₄₉₀).

A compound (L-4) wherein T represents a group represented by T7, R¹ represents S(O)CF₃, R^{3c} represents a hydrogen atom, and R^{3b} represents any substituents indicated in [Table L4] to [Table L12] (hereinafter, referred to as Compound Class SX₁₄₉₁).

A compound (L-4) wherein T represents a group represented by T7, R¹ represents S(O)₂CF₃, R^{3c} represents a hydrogen atom, and R^{3b} represents any substituents indicated in [Table L4] to [Table L12] (hereinafter, referred to as Compound Class SX₁₄₉₂).

A compound (L-4) wherein T represents a group represented by T7, R¹ represents CF₃, R^{3b} represents a hydrogen atom, and R^{3c} represents any substituents indicated in [Table L4] to [Table L12] (hereinafter, referred to as Compound Class SX₁₄₉₃).

A compound (L-4) wherein T represents a group represented by T7, R¹ represents C₂F₅, R^{3b} represents a hydrogen atom, and R^{3c} represents any substituents indicated in [Table L4] to [Table L12] (hereinafter, referred to as Compound Class SX₁₄₉₄).

A compound (L-4) wherein T represents a group represented by T7, R¹ represents SCF₃, R^{3b} represents a hydrogen atom, and R^{3c} represents any substituents indicated in [Table L4] to [Table L12] (hereinafter, referred to as Compound Class SX₁₄₉₅).

A compound (L-4) wherein T represents a group represented by T7, R¹ represents S(O)CF₃, R^{3b} represents a hydrogen atom, and R^{3c} represents any substituents indicated in [Table L4] to [Table L12] (hereinafter, referred to as Compound Class SX₁₄₉₆).

A compound (L-4) wherein T represents a group represented by T7, R¹ represents S(O)₂CF₃, R^{3b} represents a hydrogen atom, and R^{3c} represents any substituents indicated in [Table L4] to [Table L12] (hereinafter, referred to as Compound Class SX₁₄₉₇).

A compound (L-4) wherein T represents a group represented by T8, R¹ represents CF₃, R^{3c} represents a hydrogen atom, and R^{3b} represents any substituents indicated in [Table L4] to [Table L12] (hereinafter, referred to as Compound Class SX₁₄₉₈).

A compound (L-4) wherein T represents a group represented by T8, R¹ represents C₂F₅, R^{3c} represents a hydrogen atom, and R^{3b} represents any substituents indicated in [Table L4] to [Table L12] (hereinafter, referred to as Compound Class SX₁₄₉₉).

A compound (L-4) wherein T represents a group represented by T8, R¹ represents SCF₃, R^{3c} represents a hydrogen atom, and R^{3b} represents any substituents indicated in [Table L4] to [Table L12] (hereinafter, referred to as Compound Class SX₁₅₀₀).

A compound (L-4) wherein T represents a group represented by T8, R¹ represents S(O)CF₃, R^{3c} represents a hydrogen atom, and R^{3b} represents any substituents indicated in [Table L4] to [Table L12] (hereinafter, referred to as Compound Class SX₁₅₀₁).

A compound (L-4) wherein T represents a group represented by T8, R¹ represents S(O)₂CF₃, R^{3c} represents a hydrogen atom, and R^{3b} represents any substituents indicated in [Table L4] to [Table L12] (hereinafter, referred to as Compound Class SX₁₅₀₂).

A compound (L-4) wherein T represents a group represented by T8, R¹ represents CF₃, R^{3b} represents a hydrogen atom, and R^{3c} represents any substituents indicated in [Table L4] to [Table L12] (hereinafter, referred to as Compound Class SX₁₅₀₃).

A compound (L-4) wherein T represents a group represented by T8, R¹ represents C₂F₅, R^{3b} represents a hydrogen atom, and R^{3c} represents any substituents indicated in [Table L4] to [Table L12] (hereinafter, referred to as Compound Class SX₁₅₀₄).

A compound (L-4) wherein T represents a group represented by T8, R¹ represents SCF₃, R^{3b} represents a hydrogen atom, and R^{3c} represents any substituents indicated in [Table L4] to [Table L12] (hereinafter, referred to as Compound Class SX₁₅₀₅).

A compound (L-4) wherein T represents a group represented by T8, R¹ represents S(O)CF₃, R^{3b} represents a hydrogen atom, and R^{3c} represents any substituents indicated in [Table L4] to [Table L12] (hereinafter, referred to as Compound Class SX₁₅₀₆).

A compound (L-4) wherein T represents a group represented by T8, R¹ represents S(O)₂CF₃, R^{3b} represents a hydrogen atom, and R^{3c} represents any substituents indicated in [Table L4] to [Table L12] (hereinafter, referred to as Compound Class SX₁₅₀₇).

A compound (L-4) wherein T represents a group represented by T11, R¹ represents CF₃, R^{3c} represents a hydrogen atom, and R^{3b} represents any substituents indicated in [Table L4] to [Table L12] (hereinafter, referred to as Compound Class SX₁₅₀₈).

A compound (L-4) wherein T represents a group represented by T11, R¹ represents C₂F₅, R^{3c} represents a hydrogen atom, and R^{3b} represents any substituents indicated in [Table L4] to [Table L12] (hereinafter, referred to as Compound Class SX₁₅₀₉).

A compound (L-4) wherein T represents a group represented by T11, R¹ represents SCF₃, R^{3c} represents a hydrogen atom, and R^{3b} represents any substituents indicated in [Table L4] to [Table L12] (hereinafter, referred to as Compound Class SX₁₅₁₀).

A compound (L-4) wherein T represents a group represented by T11, R¹ represents S(O)CF₃, R^{3c} represents a hydrogen atom, and R^{3b} represents any substituents indicated in [Table L4] to [Table L12] (hereinafter, referred to as Compound Class SX₁₅₁₁).

A compound (L-4) wherein T represents a group represented by T11, R¹ represents S(O)₂CF₃, R^{3c} represents a hydrogen atom, and R^{3b} represents any substituents indicated in [Table L4] to [Table L12] (hereinafter, referred to as Compound Class SX₁₅₁₂).

A compound (L-4) wherein T represents a group represented by T11, R¹ represents CF₃, R^{3b} represents a hydrogen atom, and R^{3c} represents any substituents indicated in [Table L4] to [Table L12] (hereinafter, referred to as Compound Class SX₁₅₁₃).

A compound (L-4) wherein T represents a group represented by T11, R¹ represents C₂F₅, R^{3b} represents a hydrogen atom, and R^{3c} represents any substituents indicated in [Table L4] to [Table L12] (hereinafter, referred to as Compound Class SX₁₅₁₄).

A compound (L-4) wherein T represents a group represented by T11, R¹ represents SCF₃, R^{3b} represents a hydrogen atom, and R^{3c} represents any substituents indicated in [Table L4] to [Table L12] (hereinafter, referred to as Compound Class SX₁₅₁₅).

A compound (L-4) wherein T represents a group represented by T11, R¹ represents S(O)CF₃, R^{3b} represents a hydrogen atom, and R^{3c} represents any substituents indicated in [Table L4] to [Table L12] (hereinafter, referred to as Compound Class SX₁₅₁₆).

A compound (L-4) wherein T represents a group represented by T11, R¹ represents S(O)₂CF₃, R^{3b} represents a hydrogen atom, and R^{3c} represents any substituents indicated in [Table L4] to [Table L12] (hereinafter, referred to as Compound Class SX₁₅₁₇).

A compound (L-4) wherein T represents a group represented by T12, R¹ represents CF₃, R^{3c} represents a hydrogen atom, and R^{3b} represents any substituents indicated in [Table L4] to [Table L12] (hereinafter, referred to as Compound Class SX₁₅₁₈).

A compound (L-4) wherein T represents a group represented by T12, R¹ represents C₂F₅, R^{3c} represents a hydrogen atom, and R^{3b} represents any substituents indicated in [Table L4] to [Table L12] (hereinafter, referred to as Compound Class SX₁₅₁₉).

A compound (L-4) wherein T represents a group represented by T12, R¹ represents SCF₃, R^{3c} represents a hydrogen atom, and R^{3b} represents any substituents indicated in [Table L4] to [Table L12] (hereinafter, referred to as Compound Class SX₁₅₂₀).

A compound (L-4) wherein T represents a group represented by T12, R¹ represents S(O)CF₃, R^{3c} represents a hydrogen atom, and R^{3b} represents any substituents indicated in [Table L4] to [Table L12] (hereinafter, referred to as Compound Class SX₁₅₂₁).

A compound (L-4) wherein T represents a group represented by T12, R¹ represents S(O)₂CF₃, R^{3c} represents a hydrogen atom, and R^{3b} represents any substituents indicated in [Table L4] to [Table L12] (hereinafter, referred to as Compound Class SX₁₅₂₂).

A compound (L-4) wherein T represents a group represented by T12, R¹ represents CF₃, R^{3b} represents a hydrogen atom, and R^{3c} represents any substituents indicated in [Table L4] to [Table L12] (hereinafter, referred to as Compound Class SX₁₅₂₃).

A compound (L-4) wherein T represents a group represented by T12, R¹ represents C₂F₅, R^{3b} represents a hydrogen atom, and R^{3c} represents any substituents indicated in [Table L4] to [Table L12] (hereinafter, referred to as Compound Class SX₁₅₂₄).

A compound (L-4) wherein T represents a group represented by T12, R¹ represents SCF₃, R^{3b} represents a hydrogen atom, and R^{3c} represents any substituents indicated in [Table L4] to [Table L12] (hereinafter, referred to as Compound Class SX₁₅₂₅).

A compound (L-4) wherein T represents a group represented by T12, R¹ represents S(O)CF₃, R^{3b} represents a hydrogen atom, and R^{3c} represents any substituents indicated in [Table L4] to [Table L12] (hereinafter, referred to as Compound Class SX₁₅₂₆).

A compound (L-4) wherein T represents a group represented by T12, R¹ represents S(O)₂CF₃, R^{3b} represents a hydrogen atom, and R^{3c} represents any substituents indicated in [Table L4] to [Table L12] (hereinafter, referred to as Compound Class SX₁₅₂₇).

A compound (L-4) wherein T represents a group represented by T13, R¹ represents CF₃, R^{3c} represents a hydrogen atom, and R^{3b} represents any substituents indicated in [Table L4] to [Table L12] (hereinafter, referred to as Compound Class SX₁₅₂₈).

A compound (L-4) wherein T represents a group represented by T13, R¹ represents C₂F₅, R^{3c} represents a hydrogen atom, and R^{3b} represents any substituents indicated in [Table L4] to [Table L12] (hereinafter, referred to as Compound Class SX₁₅₂₉).

A compound (L-4) wherein T represents a group represented by T13, R¹ represents SCF₃, R^{3c} represents a hydrogen atom, and R^{3b} represents any substituents indicated in [Table L4] to [Table L12] (hereinafter, referred to as Compound Class SX₁₅₃₀).

A compound (L-4) wherein T represents a group represented by T13, R¹ represents S(O)CF₃, R^{3c} represents a hydrogen atom, and R^{3b} represents any substituents indicated in [Table L4] to [Table L12] (hereinafter, referred to as Compound Class SX₁₅₃₁).

A compound (L-4) wherein T represents a group represented by T13, R¹ represents S(O)₂CF₃, R^{3c} represents a hydrogen atom, and R^{3b} represents any substituents indicated in [Table L4] to [Table L12] (hereinafter, referred to as Compound Class SX₁₅₃₂).

A compound (L-4) wherein T represents a group represented by T13, R¹ represents CF₃, R^{3b} represents a hydrogen atom, and R^{3c} represents any substituents indicated in [Table L4] to [Table L12] (hereinafter, referred to as Compound Class SX₁₅₃₃).

A compound (L-4) wherein T represents a group represented by T13, R¹ represents C₂F₅, R^{3b} represents a hydrogen atom, and R^{3c} represents any substituents indicated in [Table L4] to [Table L12] (hereinafter, referred to as Compound Class SX₁₅₃₄).

A compound (L-4) wherein T represents a group represented by T13, R¹ represents SCF₃, R^{3b} represents a hydrogen atom, and R^{3c} represents any substituents indicated in [Table L4] to [Table L12] (hereinafter, referred to as Compound Class SX₁₅₃₅).

A compound (L-4) wherein T represents a group represented by T13, R¹ represents S(O)CF₃, R^{3b} represents a hydrogen atom, and R^{3c} represents any substituents indicated in [Table L4] to [Table L12] (hereinafter, referred to as Compound Class SX₁₅₃₆).

A compound (L-4) wherein T represents a group represented by T13, R¹ represents S(O)₂CF₃, R^{3b} represents a hydrogen atom, and R^{3c} represents any substituents indicated in [Table L4] to [Table L12] (hereinafter, referred to as Compound Class SX₁₅₃₇).

A compound (L-5) wherein T represents a group represented by T1, R¹ represents CF₃, and R^{3c} represents any substituents indicated in [Table L4] to [Table L12](hereinafter, referred to as Compound Class SX₁₅₃₈).

A compound (L-5) wherein T represents a group represented by T1, R¹ represents C₂F₅, and R^{3c} represents any substituents indicated in [Table L4] to [Table L12](hereinafter, referred to as Compound Class SX₁₅₃₉).

A compound (L-5) wherein T represents a group represented by T1, R¹ represents SCF₃, and R^{3c} represents any substituents indicated in [Table L4] to [Table L12](hereinafter, referred to as Compound Class SX₁₅₄₀).

A compound (L-5) wherein T represents a group represented by T1, R¹ represents S(O)CF₃, and R^{3c} represents any substituents indicated in [Table L4] to [Table L12](hereinafter, referred to as Compound Class SX₁₅₄₁).

A compound (L-5) wherein T represents a group represented by T1, R¹ represents S(O)₂CF₃, and R^{3c} represents any substituents indicated in [Table L4] to [Table L12](hereinafter, referred to as Compound Class SX₁₅₄₂).

A compound (L-5) wherein T represents a group represented by T2, R¹ represents CF₃, and R^{3c} represents any substituents indicated in [Table L4] to [Table L12](hereinafter, referred to as Compound Class SX₁₅₄₃).

A compound (L-5) wherein T represents a group represented by T2, R¹ represents C₂F₅, and R^{3c} represents any substituents indicated in [Table L4] to [Table L12](hereinafter, referred to as Compound Class SX₁₅₄₄).

A compound (L-5) wherein T represents a group represented by T2, R¹ represents SCF₃, and R^{3c} represents any substituents indicated in [Table L4] to [Table L12](hereinafter, referred to as Compound Class SX₁₅₄₅).

A compound (L-5) wherein T represents a group represented by T2, R¹ represents S(O)CF₃, and R^{3c} represents any substituents indicated in [Table L4] to [Table L12](hereinafter, referred to as Compound Class SX₁₅₄₆).

A compound (L-5) wherein T represents a group represented by T2, R¹ represents S(O)₂CF₃, and R^{3c} represents any substituents indicated in [Table L4] to [Table L12](hereinafter, referred to as Compound Class SX₁₅₄₇).

A compound (L-5) wherein T represents a group represented by T3, R¹ represents CF₃, and R^{3c} represents any substituents indicated in [Table L4] to [Table L12](hereinafter, referred to as Compound Class SX₁₅₄₈).

A compound (L-5) wherein T represents a group represented by T3, R¹ represents C₂F₅, and R^{3c} represents any substituents indicated in [Table L4] to [Table L12](hereinafter, referred to as Compound Class SX₁₅₄₉).

A compound (L-5) wherein T represents a group represented by T3, R¹ represents SCF₃, and R^{3c} represents any substituents indicated in [Table L4] to [Table L12](hereinafter, referred to as Compound Class SX₁₅₅₀).

A compound (L-5) wherein T represents a group represented by T3, R¹ represents S(O)CF₃, and R^{3c} represents any substituents indicated in [Table L4] to [Table L12](hereinafter, referred to as Compound Class SX₁₅₅₁).

A compound (L-5) wherein T represents a group represented by T3, R¹ represents S(O)₂CF₃, and R^{3c} represents any substituents indicated in [Table L4] to [Table L12](hereinafter, referred to as Compound Class SX₁₅₅₂).

A compound (L-5) wherein T represents a group represented by T6, R¹ represents CF₃, and R^{3c} represents any substituents indicated in [Table L4] to [Table L12](hereinafter, referred to as Compound Class SX₁₅₅₃).

A compound (L-5) wherein T represents a group represented by T6, R¹ represents C₂F₅, and R^{3c} represents any substituents indicated in [Table L4] to [Table L12](hereinafter, referred to as Compound Class SX₁₅₅₄).

A compound (L-5) wherein T represents a group represented by T6, R¹ represents SCF₃, and R^{3c} represents any substituents indicated in [Table L4] to [Table L12](hereinafter, referred to as Compound Class SX₁₅₅₅).

A compound (L-5) wherein T represents a group represented by T6, R¹ represents S(O)CF₃, and R^{3c} represents any substituents indicated in [Table L4] to [Table L12](hereinafter, referred to as Compound Class SX₁₅₅₆).

A compound (L-5) wherein T represents a group represented by T6, R¹ represents S(O)₂CF₃, and R^{3c} represents any substituents indicated in [Table L4] to [Table L12](hereinafter, referred to as Compound Class SX₁₅₅₇).

A compound (L-5) wherein T represents a group represented by T7, R¹ represents CF₃, and R^{3c} represents any substituents indicated in [Table L4] to [Table L12](hereinafter, referred to as Compound Class SX₁₅₅₈).

A compound (L-5) wherein T represents a group represented by T7, R¹ represents C₂F₅, and R^{3c} represents any substituents indicated in [Table L4] to [Table L12](hereinafter, referred to as Compound Class SX₁₅₅₉).

A compound (L-5) wherein T represents a group represented by T7, R¹ represents SCF₃, and R^{3c} represents any substituents indicated in [Table L4] to [Table L12](hereinafter, referred to as Compound Class SX₁₅₆₀).

A compound (L-5) wherein T represents a group represented by T7, R¹ represents S(O)CF₃, and R^{3c} represents any substituents indicated in [Table L4] to [Table L12](hereinafter, referred to as Compound Class SX₁₅₆₁).

A compound (L-5) wherein T represents a group represented by T7, R¹ represents S(O)₂CF₃, and R^{3c} represents any substituents indicated in [Table L4] to [Table L12](hereinafter, referred to as Compound Class SX₁₅₆₂).

A compound (L-5) wherein T represents a group represented by T8, R¹ represents CF₃, and R^{3c} represents any substituents indicated in [Table L4] to [Table L12](hereinafter, referred to as Compound Class SX₁₅₆₃).

A compound (L-5) wherein T represents a group represented by T8, R¹ represents C₂F₅, and R^{3c} represents any substituents indicated in [Table L4] to [Table L12](hereinafter, referred to as Compound Class SX₁₅₆₄).

A compound (L-5) wherein T represents a group represented by T8, R¹ represents SCF₃, and R^{3c} represents any substituents indicated in [Table L4] to [Table L12](hereinafter, referred to as Compound Class SX₁₅₆₅).

A compound (L-5) wherein T represents a group represented by T8, R¹ represents S(O)CF₃, and R^{3c} represents any substituents indicated in [Table L4] to [Table L12](hereinafter, referred to as Compound Class SX₁₅₆₆).

A compound (L-5) wherein T represents a group represented by T8, R¹ represents S(O)₂CF₃, and R^{3c} represents any substituents indicated in [Table L4] to [Table L12](hereinafter, referred to as Compound Class SX₁₅₆₇).

A compound (L-5) wherein T represents a group represented by T11, R¹ represents CF₃, and R^{3c} represents any substituents indicated in [Table L4] to [Table L12](hereinafter, referred to as Compound Class SX₁₅₆₈).

A compound (L-5) wherein T represents a group represented by T11, R¹ represents C₂F₅, and R^{3c} represents any substituents indicated in [Table L4] to [Table L12](hereinafter, referred to as Compound Class SX₁₅₆₉).

A compound (L-5) wherein T represents a group represented by T11, R¹ represents SCF₃, and R^{3c} represents any substituents indicated in [Table L4] to [Table L12](hereinafter, referred to as Compound Class SX₁₅₇₀).

A compound (L-5) wherein T represents a group represented by T11, R¹ represents S(O)CF₃, and R^{3c} represents any substituents indicated in [Table L4] to [Table L12](hereinafter, referred to as Compound Class SX₁₅₇₁).

A compound (L-5) wherein T represents a group represented by T11, R¹ represents S(O)₂CF₃, and R^{3c} represents any substituents indicated in [Table L4] to [Table L12](hereinafter, referred to as Compound Class SX₁₅₇₂).

A compound (L-5) wherein T represents a group represented by T12, R¹ represents CF₃, and R^{3c} represents any substituents indicated in [Table L4] to [Table L12](hereinafter, referred to as Compound Class SX₁₅₇₃).

A compound (L-5) wherein T represents a group represented by T12, R¹ represents C₂F₅, and R^{3c} represents any substituents indicated in [Table L4] to [Table L12](hereinafter, referred to as Compound Class SX₁₅₇₄).

A compound (L-5) wherein T represents a group represented by T12, R¹ represents SCF₃, and R^{3c} represents any substituents indicated in [Table L4] to [Table L12](hereinafter, referred to as Compound Class SX₁₅₇₅).

A compound (L-5) wherein T represents a group represented by T12, R¹ represents S(O)CF₃, and R^{3c} represents any substituents indicated in [Table L4] to [Table L12](hereinafter, referred to as Compound Class SX₁₅₇₆).

A compound (L-5) wherein T represents a group represented by T12, R¹ represents S(O)₂CF₃, and R^{3c} represents any substituents indicated in [Table L4] to [Table L12](hereinafter, referred to as Compound Class SX₁₅₇₇).

A compound (L-5) wherein T represents a group represented by T13, R¹ represents CF₃, and R^{3c} represents any substituents indicated in [Table L4] to [Table L12](hereinafter, referred to as Compound Class SX₁₅₇₈).

A compound (L-5) wherein T represents a group represented by T13, R¹ represents C₂F₅, and R^{3c} represents any substituents indicated in [Table L4] to [Table L12](hereinafter, referred to as Compound Class SX₁₅₇₉).

A compound (L-5) wherein T represents a group represented by T13, R¹ represents SCF₃, and R^{3c} represents any substituents indicated in [Table L4] to [Table L12](hereinafter, referred to as Compound Class SX₁₅₈₀).

A compound (L-5) wherein T represents a group represented by T13, R¹ represents S(O)CF₃, and R^{3c} represents any substituents indicated in [Table L4] to [Table L12](hereinafter, referred to as Compound Class SX₁₅₈₁).

A compound (L-5) wherein T represents a group represented by T13, R¹ represents S(O)₂CF₃, and R^{3c} represents any substituents indicated in [Table L4] to [Table L12](hereinafter, referred to as Compound Class SX₁₅₈₂).

A compound (L-6) wherein T represents a group represented by T1, R¹ represents CF₃, and R^{3b} represents any substituents indicated in [Table L4] to [Table L12](hereinafter, referred to as Compound Class SX₁₅₈₃).

A compound (L-6) wherein T represents a group represented by T1, R¹ represents C₂F₅, and R^{3b} represents any substituents indicated in [Table L4] to [Table L12](hereinafter, referred to as Compound Class SX₁₅₈₄).

A compound (L-6) wherein T represents a group represented by T1, R¹ represents SCF₃, and R^{3b} represents any substituents indicated in [Table L4] to [Table L12](hereinafter, referred to as Compound Class SX₁₅₈₅).

A compound (L-6) wherein T represents a group represented by T1, R¹ represents S(O)CF₃, and R^{3b} represents any substituents indicated in [Table L4] to [Table L12](hereinafter, referred to as Compound Class SX₁₅₈₆).

A compound (L-6) wherein T represents a group represented by T1, R¹ represents S(O)₂CF₃, and R^{3b} represents any substituents indicated in [Table L4] to [Table L12](hereinafter, referred to as Compound Class SX₁₅₈₇).

A compound (L-6) wherein T represents a group represented by T2, R¹ represents CF₃, and R^{3b} represents any substituents indicated in [Table L4] to [Table L12](hereinafter, referred to as Compound Class SX₁₅₈₈).

A compound (L-6) wherein T represents a group represented by T2, R¹ represents C₂F₅, and R^{3b} represents any substituents indicated in [Table L4] to [Table L12](hereinafter, referred to as Compound Class SX₁₅₈₉).

A compound (L-6) wherein T represents a group represented by T2, R¹ represents SCF₃, and R^{3b} represents any substituents indicated in [Table L4] to [Table L12](hereinafter, referred to as Compound Class SX₁₅₉₀).

A compound (L-6) wherein T represents a group represented by T2, R¹ represents S(O)CF₃, and R^{3b} represents any substituents indicated in [Table L4] to [Table L12](hereinafter, referred to as Compound Class SX₁₅₉₁).

A compound (L-6) wherein T represents a group represented by T2, R¹ represents S(O)₂CF₃, and R^{3b} represents any substituents indicated in [Table L4] to [Table L12](hereinafter, referred to as Compound Class SX₁₅₉₂).

A compound (L-6) wherein T represents a group represented by T3, R¹ represents CF₃, and R^{3b} represents any substituents indicated in [Table L4] to [Table L12](hereinafter, referred to as Compound Class SX₁₅₉₃).

A compound (L-6) wherein T represents a group represented by T3, R¹ represents C₂F₅, and R^{3b} represents any substituents indicated in [Table L4] to [Table L12](hereinafter, referred to as Compound Class SX₁₅₉₄).

A compound (L-6) wherein T represents a group represented by T3, R¹ represents SCF₃, and R^{3b} represents any substituents indicated in [Table L4] to [Table L12](hereinafter, referred to as Compound Class SX₁₅₉₅).

A compound (L-6) wherein T represents a group represented by T3, R¹ represents S(O)CF₃, and R^{3b} represents any substituents indicated in [Table L4] to [Table L12](hereinafter, referred to as Compound Class SX₁₅₉₆).

A compound (L-6) wherein T represents a group represented by T3, R¹ represents S(O)₂CF₃, and R^{3b} represents any substituents indicated in [Table L4] to [Table L12](hereinafter, referred to as Compound Class SX₁₅₉₇).

A compound (L-6) wherein T represents a group represented by T6, R¹ represents CF₃, and R^{3b} represents any substituents indicated in [Table L4] to [Table L12](hereinafter, referred to as Compound Class SX₁₅₉₈).

A compound (L-6) wherein T represents a group represented by T6, R¹ represents C₂F₅, and R^{3b} represents any substituents indicated in [Table L4] to [Table L12](hereinafter, referred to as Compound Class SX₁₅₉₉).

A compound (L-6) wherein T represents a group represented by T6, R¹ represents SCF₃, and R^{3b} represents any substituents indicated in [Table L4] to [Table L12](hereinafter, referred to as Compound Class SX₁₆₀₀).

A compound (L-6) wherein T represents a group represented by T6, R¹ represents S(O)CF₃, and R^{3b} represents any substituents indicated in [Table L4] to [Table L12](hereinafter, referred to as Compound Class SX₁₆₀₁).

A compound (L-6) wherein T represents a group represented by T6, R¹ represents S(O)₂CF₃, and R^{3b} represents any substituents indicated in [Table L4] to [Table L12](hereinafter, referred to as Compound Class SX₁₆₀₂).

A compound (L-6) wherein T represents a group represented by T7, R¹ represents CF₃, and R^{3b} represents any substituents indicated in [Table L4] to [Table L12](hereinafter, referred to as Compound Class SX₁₆₀₃).

A compound (L-6) wherein T represents a group represented by T7, R¹ represents C₂F₅, and R^{3b} represents any substituents indicated in [Table L4] to [Table L12](hereinafter, referred to as Compound Class SX₁₆₀₄).

A compound (L-6) wherein T represents a group represented by T7, R¹ represents SCF₃, and R^{3b} represents any substituents indicated in [Table L4] to [Table L12](hereinafter, referred to as Compound Class SX₁₆₀₅).

A compound (L-6) wherein T represents a group represented by T7, R¹ represents S(O)CF₃, and R^{3b} represents any substituents indicated in [Table L4] to [Table L12](hereinafter, referred to as Compound Class SX₁₆₀₆).

A compound (L-6) wherein T represents a group represented by T7, R¹ represents S(O)₂CF₃, and R^{3b} represents any substituents indicated in [Table L4] to [Table L12](hereinafter, referred to as Compound Class SX₁₆₀₇).

A compound (L-6) wherein T represents a group represented by T8, R¹ represents CF₃, and R^{3b} represents any substituents indicated in [Table L4] to [Table L12](hereinafter, referred to as Compound Class SX₁₆₀₈).

A compound (L-6) wherein T represents a group represented by T8, R¹ represents C₂F₅, and R^{3b} represents any substituents indicated in [Table L4] to [Table L12](hereinafter, referred to as Compound Class SX₁₆₀₉).

A compound (L-6) wherein T represents a group represented by T8, R¹ represents SCF₃, and R^{3b} represents any substituents indicated in [Table L4] to [Table L12](hereinafter, referred to as Compound Class SX₁₆₁₀).

A compound (L-6) wherein T represents a group represented by T8, R¹ represents S(O)CF₃, and R^{3b} represents any substituents indicated in [Table L4] to [Table L12](hereinafter, referred to as Compound Class SX₁₆₁₁).

A compound (L-6) wherein T represents a group represented by T8, R¹ represents S(O)₂CF₃, and R^{3b} represents any substituents indicated in [Table L4] to [Table L12](hereinafter, referred to as Compound Class SX₁₆₁₂).

A compound (L-6) wherein T represents a group represented by T9, R¹ represents CF₃, and R^{3b} represents any substituents indicated in [Table L4] to [Table L12](hereinafter, referred to as Compound Class SX₁₆₁₃).

A compound (L-6) wherein T represents a group represented by T11, R¹ represents CF₃, and R^{3b} represents any substituents indicated in [Table L4] to [Table L12](hereinafter, referred to as Compound Class SX₁₆₁₄).

A compound (L-6) wherein T represents a group represented by T11, R¹ represents C₂F₅, and R^{3b} represents any substituents indicated in [Table L4] to [Table L12](hereinafter, referred to as Compound Class SX₁₆₁₅).

A compound (L-6) wherein T represents a group represented by T11, R¹ represents SCF₃, and R^{3b} represents any substituents indicated in [Table L4] to [Table L12](hereinafter, referred to as Compound Class SX₁₆₁₆).

A compound (L-6) wherein T represents a group represented by T11, R¹ represents S(O)CF₃, and R^{3b} represents any substituents indicated in [Table L4] to [Table L12](hereinafter, referred to as Compound Class SX₁₆₁₇).

A compound (L-6) wherein T represents a group represented by T11, R¹ represents S(O)₂CF₃, and R^{3b} represents any substituents indicated in [Table L4] to [Table L12](hereinafter, referred to as Compound Class SX₁₆₁₈).

A compound (L-6) wherein T represents a group represented by T12, R¹ represents CF₃, and R^{3b} represents any substituents indicated in [Table L4] to [Table L12](hereinafter, referred to as Compound Class SX₁₆₁₉).

A compound (L-6) wherein T represents a group represented by T12, R¹ represents C₂F₅, and R^{3b} represents any substituents indicated in [Table L4] to [Table L12](hereinafter, referred to as Compound Class SX₁₆₂₀).

A compound (L-6) wherein T represents a group represented by T12, R¹ represents SCF₃, and R^{3b} represents any substituents indicated in [Table L4] to [Table L12](hereinafter, referred to as Compound Class SX₁₆₂₁).

A compound (L-6) wherein T represents a group represented by T12, R¹ represents S(O)CF₃, and R^{3b} represents any substituents indicated in [Table L4] to [Table L12](hereinafter, referred to as Compound Class SX₁₆₂₂).

A compound (L-6) wherein T represents a group represented by T12, R¹ represents S(O)₂CF₃, and R^{3b} represents any substituents indicated in [Table L4] to [Table L12](hereinafter, referred to as Compound Class SX₁₆₂₃).

A compound (L-6) wherein T represents a group represented by T13, R¹ represents CF₃, and R^{3b} represents any substituents indicated in [Table L4] to [Table L12](hereinafter, referred to as Compound Class SX₁₆₂₄).

A compound (L-6) wherein T represents a group represented by T13, R¹ represents C₂F₅, and R^{3b} represents any substituents indicated in [Table L4] to [Table L12](hereinafter, referred to as Compound Class SX₁₆₂₅).

A compound (L-6) wherein T represents a group represented by T13, R¹ represents SCF₃, and R^{3b} represents any substituents indicated in [Table L4] to [Table L12](hereinafter, referred to as Compound Class SX₁₆₂₆).

A compound (L-6) wherein T represents a group represented by T13, R¹ represents S(O)CF₃, and R^{3b} represents any substituents indicated in [Table L4] to [Table L12](hereinafter, referred to as Compound Class SX₁₆₂₇).

A compound (L-6) wherein T represents a group represented by T13, R¹ represents S(O)₂CF₃, and R^{3b} represents any substituents indicated in [Table L4] to [Table L12](hereinafter, referred to as Compound Class SX₁₆₂₈).

A compound represented by formula (L-7):

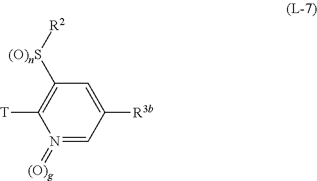

(hereinafter, referred to as compound (L-7), wherein T represents a group represented by T1, R¹ represents CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₂₉).

A compound (L-7) wherein T represents a group represented by T1, R¹ represents CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₃₀).

A compound (L-7) wherein T represents a group represented by T1, R¹ represents CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₃₁).

A compound (L-7) wherein T represents a group represented by T1, R¹ represents CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₆₃₂).

A compound (L-7) wherein T represents a group represented by T1, R¹ represents CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₃₃).

A compound (L-7) wherein T represents a group represented by T1, R¹ represents CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₃₄).

A compound (L-7) wherein T represents a group represented by T1, R¹ represents CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₆₃₅).

A compound (L-7) wherein T represents a group represented by T2, R¹ represents CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₃₆).

A compound (L-7) wherein T represents a group represented by T2, R¹ represents CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₃₇).

A compound (L-7) wherein T represents a group represented by T2, R¹ represents CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₃₈).

A compound (L-7) wherein T represents a group represented by T2, R¹ represents CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₆₃₉).

A compound (L-7) wherein T represents a group represented by T2, R¹ represents CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₄₀).

A compound (L-7) wherein T represents a group represented by T2, R¹ represents CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₄₁).

A compound (L-7) wherein T represents a group represented by T2, R¹ represents CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₆₄₂).

A compound (L-7) wherein T represents a group represented by T3, R¹ represents CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₄₃).

A compound (L-7) wherein T represents a group represented by T3, R¹ represents CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₄₄).

A compound (L-7) wherein T represents a group represented by T3, R¹ represents CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₄₅).

A compound (L-7) wherein T represents a group represented by T3, R¹ represents CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₆₄₆).

A compound (L-7) wherein T represents a group represented by T3, R¹ represents CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₄₇).

A compound (L-7) wherein T represents a group represented by T3, R¹ represents CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₄₈).

A compound (L-7) wherein T represents a group represented by T3, R¹ represents CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₆₄₉).

A compound (L-7) wherein T represents a group represented by T4, R¹ represents CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₅₀).

A compound (L-7) wherein T represents a group represented by T4, R¹ represents CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₅₁).

A compound (L-7) wherein T represents a group represented by T4, R¹ represents CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₅₂).

A compound (L-7) wherein T represents a group represented by T4, R¹ represents CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₆₅₃).

A compound (L-7) wherein T represents a group represented by T4, R¹ represents CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₅₄).

A compound (L-7) wherein T represents a group represented by T4, R¹ represents CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₅₅).

A compound (L-7) wherein T represents a group represented by T4, R¹ represents CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₆₅₆).

A compound (L-7) wherein T represents a group represented by T5, R¹ represents CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₅₇).

A compound (L-7) wherein T represents a group represented by T5, R¹ represents CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₅₈).

A compound (L-7) wherein T represents a group represented by T5, R¹ represents CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₅₉).

A compound (L-7) wherein T represents a group represented by T5, R¹ represents CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₆₆₀).

A compound (L-7) wherein T represents a group represented by T5, R¹ represents CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₆₁).

A compound (L-7) wherein T represents a group represented by T5, R¹ represents CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₆₂).

A compound (L-7) wherein T represents a group represented by T5, R¹ represents CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₆₆₃).

A compound (L-7) wherein T represents a group represented by T1, R¹ represents C₂F₅, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₆₄).

A compound (L-7) wherein T represents a group represented by T1, R¹ represents C₂F₅, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₆₅).

A compound (L-7) wherein T represents a group represented by T1, R¹ represents C₂F₅, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₆₆).

A compound (L-7) wherein T represents a group represented by T1, R¹ represents C₂F₅, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₆₆₇).

A compound (L-7) wherein T represents a group represented by T1, R¹ represents C₂F₅, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₆₈).

A compound (L-7) wherein T represents a group represented by T1, R¹ represents C₂F₅, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₆₉).

A compound (L-7) wherein T represents a group represented by T1, R¹ represents C₂F₅, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₆₇₀).

A compound (L-7) wherein T represents a group represented by T2, R¹ represents C₂F₅, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₇₁).

A compound (L-7) wherein T represents a group represented by T2, R¹ represents C₂F₅, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₇₂).

A compound (L-7) wherein T represents a group represented by T2, R¹ represents C₂F₅, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₇₃).

A compound (L-7) wherein T represents a group represented by T2, R¹ represents C₂F₅, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₆₇₄).

A compound (L-7) wherein T represents a group represented by T2, R¹ represents C₂F₅, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₇₅).

A compound (L-7) wherein T represents a group represented by T2, R¹ represents C₂F₅, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₇₆).

A compound (L-7) wherein T represents a group represented by T2, R¹ represents C₂F₅, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₆₇₇).

A compound (L-7) wherein T represents a group represented by T3, R¹ represents C₂F₅, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₇₈).

A compound (L-7) wherein T represents a group represented by T3, R¹ represents C₂F₅, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₇₉).

A compound (L-7) wherein T represents a group represented by T3, R¹ represents C₂F₅, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₈₀).

A compound (L-7) wherein T represents a group represented by T3, R¹ represents C₂F₅, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₆₈₁).

A compound (L-7) wherein T represents a group represented by T3, R¹ represents C₂F₅, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₈₂).

A compound (L-7) wherein T represents a group represented by T3, R¹ represents C₂F₅, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₈₃).

A compound (L-7) wherein T represents a group represented by T3, R¹ represents C₂F₅, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₆₈₄).

A compound (L-7) wherein T represents a group represented by T4, R¹ represents C₂F₅, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₈₅).

A compound (L-7) wherein T represents a group represented by T4, R¹ represents C₂F₅, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₈₆).

A compound (L-7) wherein T represents a group represented by T4, R¹ represents C₂F₅, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₈₇).

A compound (L-7) wherein T represents a group represented by T4, R¹ represents C₂F₅, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₆₈₈).

A compound (L-7) wherein T represents a group represented by T4, R¹ represents C₂F₅, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₈₉).

A compound (L-7) wherein T represents a group represented by T4, R¹ represents C₂F₅, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₉₀).

A compound (L-7) wherein T represents a group represented by T4, R¹ represents C₂F₅, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₆₉₁).

A compound (L-7) wherein T represents a group represented by T5, R¹ represents C₂F₅, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₉₂).

A compound (L-7) wherein T represents a group represented by T5, R¹ represents C₂F₅, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₉₃).

A compound (L-7) wherein T represents a group represented by T5, R¹ represents C₂F₅, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₉₄).

A compound (L-7) wherein T represents a group represented by T5, R¹ represents C₂F₅, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₆₉₅).

A compound (L-7) wherein T represents a group represented by T5, R¹ represents C₂F₅, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₉₆).

A compound (L-7) wherein T represents a group represented by T5, R¹ represents C₂F₅, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₆₉₇).

A compound (L-7) wherein T represents a group represented by T5, R¹ represents C₂F₅, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₆₉₈).

A compound (L-7) wherein T represents a group represented by T1, R¹ represents SCF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₆₉₉).

A compound (L-7) wherein T represents a group represented by T1, R¹ represents SCF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₀₀).

A compound (L-7) wherein T represents a group represented by T1, R¹ represents SCF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₀₁).

A compound (L-7) wherein T represents a group represented by T1, R¹ represents SCF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₀₂).

A compound (L-7) wherein T represents a group represented by T1, R¹ represents SCF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₀₃).

A compound (L-7) wherein T represents a group represented by T1, R¹ represents SCF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₀₄).

A compound (L-7) wherein T represents a group represented by T1, R¹ represents SCF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₀₅).

A compound (L-7) wherein T represents a group represented by T2, R¹ represents SCF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₀₆).

A compound (L-7) wherein T represents a group represented by T2, R¹ represents SCF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₀₇).

A compound (L-7) wherein T represents a group represented by T2, R¹ represents SCF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₀₈).

A compound (L-7) wherein T represents a group represented by T2, R¹ represents SCF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₀₉).

A compound (L-7) wherein T represents a group represented by T2, R¹ represents SCF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₁₀).

A compound (L-7) wherein T represents a group represented by T2, R¹ represents SCF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₁₁).

A compound (L-7) wherein T represents a group represented by T2, R¹ represents SCF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₁₂).

A compound (L-7) wherein T represents a group represented by T3, R¹ represents SCF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₁₃).

A compound (L-7) wherein T represents a group represented by T3, R¹ represents SCF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₁₄).

A compound (L-7) wherein T represents a group represented by T3, R¹ represents SCF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₁₅).

A compound (L-7) wherein T represents a group represented by T3, R¹ represents SCF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₁₆).

A compound (L-7) wherein T represents a group represented by T3, R¹ represents SCF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₁₇).

A compound (L-7) wherein T represents a group represented by T3, R¹ represents SCF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₁₈).

A compound (L-7) wherein T represents a group represented by T3, R¹ represents SCF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₁₉).

A compound (L-7) wherein T represents a group represented by T4, R¹ represents SCF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₂₀).

A compound (L-7) wherein T represents a group represented by T4, R¹ represents SCF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₂₁).

A compound (L-7) wherein T represents a group represented by T4, R¹ represents SCF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₂₂).

A compound (L-7) wherein T represents a group represented by T4, R¹ represents SCF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₂₃).

A compound (L-7) wherein T represents a group represented by T4, R¹ represents SCF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₂₄).

A compound (L-7) wherein T represents a group represented by T4, R¹ represents SCF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₂₅).

A compound (L-7) wherein T represents a group represented by T4, R¹ represents SCF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₂₆).

A compound (L-7) wherein T represents a group represented by T5, R¹ represents SCF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₂₇).

A compound (L-7) wherein T represents a group represented by T5, R¹ represents SCF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₂₈).

A compound (L-7) wherein T represents a group represented by T5, R¹ represents SCF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₂₉).

A compound (L-7) wherein T represents a group represented by T5, R¹ represents SCF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₃₀).

A compound (L-7) wherein T represents a group represented by T5, R¹ represents SCF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₃₁).

A compound (L-7) wherein T represents a group represented by T5, R¹ represents SCF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₃₂).

A compound (L-7) wherein T represents a group represented by T5, R¹ represents SCF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₃₃).

A compound (L-7) wherein T represents a group represented by T1, R¹ represents S(O)CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₃₄).

A compound (L-7) wherein T represents a group represented by T1, R¹ represents S(O)CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₃₅).

A compound (L-7) wherein T represents a group represented by T1, R¹ represents S(O)CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₃₆).

A compound (L-7) wherein T represents a group represented by T1, R¹ represents S(O)CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₃₇).

A compound (L-7) wherein T represents a group represented by T1, R¹ represents S(O)CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₃₈).

A compound (L-7) wherein T represents a group represented by T1, R¹ represents S(O)CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₃₉).

A compound (L-7) wherein T represents a group represented by T1, R¹ represents S(O)CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₄₀).

A compound (L-7) wherein T represents a group represented by T2, R¹ represents S(O)CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₄₁).

A compound (L-7) wherein T represents a group represented by T2, R¹ represents S(O)CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₄₂).

A compound (L-7) wherein T represents a group represented by T2, R¹ represents S(O)CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₄₃).

A compound (L-7) wherein T represents a group represented by T2, R¹ represents S(O)CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₄₄).

A compound (L-7) wherein T represents a group represented by T2, R¹ represents S(O)CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₄₅).

A compound (L-7) wherein T represents a group represented by T2, R¹ represents S(O)CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₄₆).

A compound (L-7) wherein T represents a group represented by T2, R¹ represents S(O)CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₄₇).

A compound (L-7) wherein T represents a group represented by T3, R¹ represents S(O)CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₄₈).

A compound (L-7) wherein T represents a group represented by T3, R¹ represents S(O)CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₄₉).

A compound (L-7) wherein T represents a group represented by T3, R¹ represents S(O)CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₅₀).

A compound (L-7) wherein T represents a group represented by T3, R¹ represents S(O)CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₅₁).

A compound (L-7) wherein T represents a group represented by T3, R¹ represents S(O)CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₅₂).

A compound (L-7) wherein T represents a group represented by T3, R¹ represents S(O)CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₅₃).

A compound (L-7) wherein T represents a group represented by T3, R¹ represents S(O)CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₅₄).

A compound (L-7) wherein T represents a group represented by T4, R¹ represents S(O)CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₅₅).

A compound (L-7) wherein T represents a group represented by T4, R¹ represents S(O)CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₅₆).

A compound (L-7) wherein T represents a group represented by T4, R¹ represents S(O)CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₅₇).

A compound (L-7) wherein T represents a group represented by T4, R¹ represents S(O)CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₅₈).

A compound (L-7) wherein T represents a group represented by T4, R¹ represents S(O)CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₅₉).

A compound (L-7) wherein T represents a group represented by T4, R¹ represents S(O)CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₆₀).

A compound (L-7) wherein T represents a group represented by T4, R¹ represents S(O)CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₆₁).

A compound (L-7) wherein T represents a group represented by T5, R¹ represents S(O)CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₆₂).

A compound (L-7) wherein T represents a group represented by T5, R¹ represents S(O)CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₆₃).

A compound (L-7) wherein T represents a group represented by T5, R¹ represents S(O)CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₆₄).

A compound (L-7) wherein T represents a group represented by T5, R¹ represents S(O)CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₆₅).

A compound (L-7) wherein T represents a group represented by T5, R¹ represents S(O)CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₆₆).

A compound (L-7) wherein T represents a group represented by T5, R¹ represents S(O)CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₆₇).

A compound (L-7) wherein T represents a group represented by T5, R¹ represents S(O)CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₆₈).

A compound (L-7) wherein T represents a group represented by T1, R¹ represents S(O)₂CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₆₉).

A compound (L-7) wherein T represents a group represented by T1, R¹ represents S(O)₂CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₇₀).

A compound (L-7) wherein T represents a group represented by T1, R¹ represents S(O)₂CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₇₁).

A compound (L-7) wherein T represents a group represented by T1, R¹ represents S(O)₂CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₇₂).

A compound (L-7) wherein T represents a group represented by T1, R¹ represents S(O)₂CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₇₃).

A compound (L-7) wherein T represents a group represented by T1, R¹ represents S(O)₂CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₇₄).

A compound (L-7) wherein T represents a group represented by T1, R¹ represents S(O)₂CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₇₅).

A compound (L-7) wherein T represents a group represented by T2, R¹ represents S(O)₂CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₇₆).

A compound (L-7) wherein T represents a group represented by T2, R¹ represents S(O)₂CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₇₇).

A compound (L-7) wherein T represents a group represented by T2, R¹ represents S(O)₂CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₇₈).

A compound (L-7) wherein T represents a group represented by T2, R¹ represents S(O)₂CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₇₉).

A compound (L-7) wherein T represents a group represented by T2, R¹ represents S(O)₂CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₈₀).

A compound (L-7) wherein T represents a group represented by T2, R¹ represents S(O)₂CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₈₁).

A compound (L-7) wherein T represents a group represented by T2, R¹ represents S(O)₂CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₈₂).

A compound (L-7) wherein T represents a group represented by T3, R¹ represents S(O)₂CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₈₃).

A compound (L-7) wherein T represents a group represented by T3, R¹ represents S(O)₂CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₈₄).

A compound (L-7) wherein T represents a group represented by T3, R¹ represents S(O)₂CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₈₅).

A compound (L-7) wherein T represents a group represented by T3, R¹ represents S(O)₂CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₈₆).

A compound (L-7) wherein T represents a group represented by T3, R¹ represents S(O)₂CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₈₇).

A compound (L-7) wherein T represents a group represented by T3, R¹ represents S(O)₂CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₈₈).

A compound (L-7) wherein T represents a group represented by T3, R¹ represents S(O)₂CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₈₉).

A compound (L-7) wherein T represents a group represented by T4, R¹ represents S(O)₂CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₉₀).

A compound (L-7) wherein T represents a group represented by T4, R¹ represents S(O)₂CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₉₁).

A compound (L-7) wherein T represents a group represented by T4, R¹ represents S(O)₂CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₉₂).

A compound (L-7) wherein T represents a group represented by T4, R¹ represents S(O)₂CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₇₉₃).

A compound (L-7) wherein T represents a group represented by T4, R¹ represents S(O)₂CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₉₄).

A compound (L-7) wherein T represents a group represented by T4, R¹ represents S(O)₂CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₇₉₅).

A compound (L-7) wherein T represents a group represented by T4, R¹ represents S(O)₂CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₇₉₆).

A compound (L-7) wherein T represents a group represented by T5, R¹ represents S(O)₂CF₃, n is 0, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₉₇).

A compound (L-7) wherein T represents a group represented by T5, R¹ represents S(O)₂CF₃, n is 1, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₉₈).

A compound (L-7) wherein T represents a group represented by T5, R¹ represents S(O)₂CF₃, n is 2, g is 0, R² represents a methyl group, and R^{3b} represents a hydrogen atom, a chlorine atom, CF₃, or any substituents indicated in [Table L4] to [Table L12] (hereinafter, referred to as Compound Class SX₁₇₉₉).

A compound (L-7) wherein T represents a group represented by T5, R¹ represents S(O)₂CF₃, n is 2, g is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₀₀).

A compound (L-7) wherein T represents a group represented by T5, R¹ represents S(O)₂CF₃, n is 0, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₀₁).

A compound (L-7) wherein T represents a group represented by T5, R¹ represents S(O)₂CF₃, n is 1, g is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₀₂).

A compound (L-7) wherein T represents a group represented by T5, R¹ represents S(O)₂CF₃, n is 2, g is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₀₃).

A compound represented by formula (L-8):

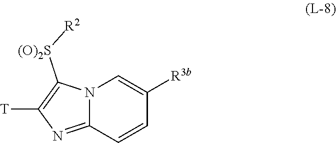

(hereinafter, referred to as compound (L-8), wherein T represents a group represented by T1, R¹ represents CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₀₄).

A compound (L-8) wherein T represents a group represented by T1, R¹ represents CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₀₅).

A compound (L-8) wherein T represents a group represented by T1, R¹ represents CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₀₆).

A compound (L-8) wherein T represents a group represented by T1, R¹ represents CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₀₇).

A compound (L-8) wherein T represents a group represented by T1, R¹ represents CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₀₈).

A compound (L-8) wherein T represents a group represented by T2, R¹ represents CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₀₉).

A compound (L-8) wherein T represents a group represented by T2, R¹ represents CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₁₀).

A compound (L-8) wherein T represents a group represented by T2, R¹ represents CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₁₁).

A compound (L-8) wherein T represents a group represented by T2, R¹ represents CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₁₂).

A compound (L-8) wherein T represents a group represented by T2, R¹ represents CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₁₃).

A compound (L-8) wherein T represents a group represented by T3, R¹ represents CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₁₄).

A compound (L-8) wherein T represents a group represented by T3, R¹ represents CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₁₅).

A compound (L-8) wherein T represents a group represented by T3, R¹ represents CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₁₆).

A compound (L-8) wherein T represents a group represented by T3, R¹ represents CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₁₇).

A compound (L-8) wherein T represents a group represented by T3, R¹ represents CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₁₈).

A compound (L-8) wherein T represents a group represented by T4, R¹ represents CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₁₉).

A compound (L-8) wherein T represents a group represented by T4, R¹ represents CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₂₀).

A compound (L-8) wherein T represents a group represented by T4, R¹ represents CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₂₁).

A compound (L-8) wherein T represents a group represented by T4, R¹ represents CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₂₂).

A compound (L-8) wherein T represents a group represented by T4, R¹ represents CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₂₃).

A compound (L-8) wherein T represents a group represented by T5, R¹ represents CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₂₄).

A compound (L-8) wherein T represents a group represented by T5, R¹ represents CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₂₅).

A compound (L-8) wherein T represents a group represented by T5, R¹ represents CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₂₆).

A compound (L-8) wherein T represents a group represented by T5, R¹ represents CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₂₇).

A compound (L-8) wherein T represents a group represented by T5, R¹ represents CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₂₈).

A compound (L-8) wherein T represents a group represented by T1, R¹ represents C₂F₅, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₂₉).

A compound (L-8) wherein T represents a group represented by T1, R¹ represents C₂F₅, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₃₀).

A compound (L-8) wherein T represents a group represented by T1, R¹ represents C₂F₅, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₃₁).

A compound (L-8) wherein T represents a group represented by T1, R¹ represents C₂F₅, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₃₂).

A compound (L-8) wherein T represents a group represented by T1, R¹ represents C₂F₅, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₃₃).

A compound (L-8) wherein T represents a group represented by T2, R¹ represents C₂F₅, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₃₄).

A compound (L-8) wherein T represents a group represented by T2, R¹ represents C₂F₅, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₃₅).

A compound (L-8) wherein T represents a group represented by T2, R¹ represents C₂F₅, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₃₆).

A compound (L-8) wherein T represents a group represented by T2, R¹ represents C₂F₅, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₃₇).

A compound (L-8) wherein T represents a group represented by T2, R¹ represents C₂F₅, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₃₈).

A compound (L-8) wherein T represents a group represented by T3, R¹ represents C₂F₅, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₃₉).

A compound (L-8) wherein T represents a group represented by T3, R¹ represents C₂F₅, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₄₀).

A compound (L-8) wherein T represents a group represented by T3, R¹ represents C₂F₅, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₄₁).

A compound (L-8) wherein T represents a group represented by T3, R¹ represents C₂F₅, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₄₂).

A compound (L-8) wherein T represents a group represented by T3, R¹ represents C₂F₅, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₄₃).

A compound (L-8) wherein T represents a group represented by T4, R¹ represents C₂F₅, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₄₄).

A compound (L-8) wherein T represents a group represented by T4, R¹ represents C₂F₅, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₄₅).

A compound (L-8) wherein T represents a group represented by T4, R¹ represents C₂F₅, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₄₆).

A compound (L-8) wherein T represents a group represented by T4, R¹ represents C₂F₅, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₄₇).

A compound (L-8) wherein T represents a group represented by T4, R¹ represents C₂F₅, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₄₈).

A compound (L-8) wherein T represents a group represented by T5, R¹ represents C₂F5, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₄₉).

A compound (L-8) wherein T represents a group represented by T5, R¹ represents C₂F5, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₅₀).

A compound (L-8) wherein T represents a group represented by T5, R¹ represents C₂F5, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₅₁).

A compound (L-8) wherein T represents a group represented by T5, R¹ represents C₂F5, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₅₂).

A compound (L-8) wherein T represents a group represented by T5, R¹ represents C₂F5, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₅₃).

A compound (L-8) wherein T represents a group represented by T1, R¹ represents SCF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₅₄).

A compound (L-8) wherein T represents a group represented by T1, R¹ represents SCF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₅₅).

A compound (L-8) wherein T represents a group represented by T1, R¹ represents SCF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₅₆).

A compound (L-8) wherein T represents a group represented by T1, R¹ represents SCF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₅₇).

A compound (L-8) wherein T represents a group represented by T1, R¹ represents SCF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₅₈).

A compound (L-8) wherein T represents a group represented by T2, R¹ represents SCF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₅₉).

A compound (L-8) wherein T represents a group represented by T2, R¹ represents SCF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₆₀).

A compound (L-8) wherein T represents a group represented by T2, R¹ represents SCF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₆₁).

A compound (L-8) wherein T represents a group represented by T2, R¹ represents SCF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₆₂).

A compound (L-8) wherein T represents a group represented by T2, R¹ represents SCF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₆₃).

A compound (L-8) wherein T represents a group represented by T3, R¹ represents SCF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₆₄).

A compound (L-8) wherein T represents a group represented by T3, R¹ represents SCF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₆₅).

A compound (L-8) wherein T represents a group represented by T3, R¹ represents SCF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₆₆).

A compound (L-8) wherein T represents a group represented by T3, R¹ represents SCF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₆₇).

A compound (L-8) wherein T represents a group represented by T3, R¹ represents SCF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₆₈).

A compound (L-8) wherein T represents a group represented by T4, R¹ represents SCF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₆₉).

A compound (L-8) wherein T represents a group represented by T4, R¹ represents SCF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₇₀).

A compound (L-8) wherein T represents a group represented by T4, R¹ represents SCF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₇₁).

A compound (L-8) wherein T represents a group represented by T4, R¹ represents SCF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₇₂).

A compound (L-8) wherein T represents a group represented by T4, R¹ represents SCF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₇₃).

A compound (L-8) wherein T represents a group represented by T5, R¹ represents SCF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₇₄).

A compound (L-8) wherein T represents a group represented by T5, R¹ represents SCF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₇₅).

A compound (L-8) wherein T represents a group represented by T5, R¹ represents SCF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₇₆).

A compound (L-8) wherein T represents a group represented by T5, R¹ represents SCF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₇₇).

A compound (L-8) wherein T represents a group represented by T5, R¹ represents SCF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₇₈).

A compound (L-8) wherein T represents a group represented by T1, R¹ represents S(O)CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₇₉).

A compound (L-8) wherein T represents a group represented by T1, R¹ represents S(O)CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₈₀).

A compound (L-8) wherein T represents a group represented by T1, R¹ represents S(O)CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₈₁).

A compound (L-8) wherein T represents a group represented by T1, R¹ represents S(O)CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₈₂).

A compound (L-8) wherein T represents a group represented by T1, R¹ represents S(O)CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₈₃).

A compound (L-8) wherein T represents a group represented by T2, R¹ represents S(O)CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₈₄).

A compound (L-8) wherein T represents a group represented by T2, R¹ represents S(O)CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₈₅).

A compound (L-8) wherein T represents a group represented by T2, R¹ represents S(O)CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₈₆).

A compound (L-8) wherein T represents a group represented by T2, R¹ represents S(O)CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₈₇).

A compound (L-8) wherein T represents a group represented by T2, R¹ represents S(O)CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₈₈).

A compound (L-8) wherein T represents a group represented by T3, R¹ represents S(O)CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₈₉).

A compound (L-8) wherein T represents a group represented by T3, R¹ represents S(O)CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₉₀).

A compound (L-8) wherein T represents a group represented by T3, R¹ represents S(O)CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₉₁).

A compound (L-8) wherein T represents a group represented by T3, R¹ represents S(O)CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₉₂).

A compound (L-8) wherein T represents a group represented by T3, R¹ represents S(O)CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₉₃).

A compound (L-8) wherein T represents a group represented by T4, R¹ represents S(O)CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₉₄).

A compound (L-8) wherein T represents a group represented by T4, R¹ represents S(O)CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₈₉₅).

A compound (L-8) wherein T represents a group represented by T4, R¹ represents S(O)CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₈₉₆).

A compound (L-8) wherein T represents a group represented by T4, R¹ represents S(O)CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₈₉₇).

A compound (L-8) wherein T represents a group represented by T4, R¹ represents S(O)CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₈₉₈).

A compound (L-8) wherein T represents a group represented by T5, R¹ represents S(O)CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₈₉₉).

A compound (L-8) wherein T represents a group represented by T5, R¹ represents S(O)CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₉₀₀).

A compound (L-8) wherein T represents a group represented by T5, R¹ represents S(O)CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₉ₒ₁).

A compound (L-8) wherein T represents a group represented by T5, R¹ represents S(O)CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₉₀₂).

A compound (L-8) wherein T represents a group represented by T5, R¹ represents S(O)CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₉₀₃).

A compound (L-8) wherein T represents a group represented by T1, R¹ represents S(O)₂CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₉₀₄).

A compound (L-8) wherein T represents a group represented by T1, R¹ represents S(O)₂CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₉₀₅).

A compound (L-8) wherein T represents a group represented by T1, R¹ represents S(O)₂CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₉₀₆).

A compound (L-8) wherein T represents a group represented by T1, R¹ represents S(O)₂CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₉₀₇).

A compound (L-8) wherein T represents a group represented by T1, R¹ represents S(O)₂CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₉₀₈).

A compound (L-8) wherein T represents a group represented by T2, R¹ represents S(O)₂CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₉₀₉).

A compound (L-8) wherein T represents a group represented by T2, R¹ represents S(O)₂CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₉₁₀).

A compound (L-8) wherein T represents a group represented by T2, R¹ represents S(O)₂CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₉₁₁).

A compound (L-8) wherein T represents a group represented by T2, R¹ represents S(O)₂CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₉₁₂).

A compound (L-8) wherein T represents a group represented by T2, R¹ represents S(O)₂CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₉₁₃).

A compound (L-8) wherein T represents a group represented by T3, R¹ represents S(O)₂CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₉₁₄).

A compound (L-8) wherein T represents a group represented by T3, R¹ represents S(O)₂CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₉₁₅).

A compound (L-8) wherein T represents a group represented by T3, R¹ represents S(O)₂CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₉₁₆).

A compound (L-8) wherein T represents a group represented by T3, R¹ represents S(O)₂CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₉₁₇).

A compound (L-8) wherein T represents a group represented by T3, R¹ represents S(O)₂CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₉₁₈).

A compound (L-8) wherein T represents a group represented by T4, R¹ represents S(O)₂CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₉₁₉).

A compound (L-8) wherein T represents a group represented by T4, R¹ represents S(O)₂CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₉₂₀).

A compound (L-8) wherein T represents a group represented by T4, R¹ represents S(O)₂CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₉₂₁).

A compound (L-8) wherein T represents a group represented by T4, R¹ represents S(O)₂CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₉₂₂).

A compound (L-8) wherein T represents a group represented by T4, R¹ represents S(O)₂CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₉₂₃).

A compound (L-8) wherein T represents a group represented by T5, R¹ represents S(O)₂CF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₁₉₂₄).

A compound (L-8) wherein T represents a group represented by T5, R¹ represents S(O)₂CF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₁₉₂₅).

A compound (L-8) wherein T represents a group represented by T5, R¹ represents S(O)₂CF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₁₉₂₆).

A compound (L-8) wherein T represents a group represented by T5, R¹ represents S(O)₂CF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₁₉₂₇).

A compound (L-8) wherein T represents a group represented by T5, R¹ represents S(O)₂CF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₁₉₂₈).

A compound (L-1) wherein T represents a group represented by T32, R^{3b} represents a hydrogen atom, and R¹ represents any substituents indicated in [Table L1] to [Table L3] (hereinafter, referred to as Compound Class SX₁₉₂₉).

A compound (L-1) wherein T represents a group represented by T32, R^{3b} represents a chlorine atom, and R¹ represents any substituents indicated in [Table L1] to [Table L3] (hereinafter, referred to as Compound Class SX₁₉₃₀).

A compound (L-1) wherein T represents a group represented by T32, R^{3b} represents a cyclopropyl group, and R¹ represents any substituents indicated in [Table L1] to [Table L3] (hereinafter, referred to as Compound Class SX₁₉₃₁).

A compound (L-1) wherein T represents a group represented by T32, R^{3b} represents a 1-cyanocyclopropyl group, and R¹ represents any substituents indicated in [Table L1] to [Table L3] (hereinafter, referred to as Compound Class SX₁₉₃₂).

A compound (L-1) wherein T represents a group represented by T32, R^{3b} represents CF₃, and R¹ represents any substituents indicated in [Table L1] to [Table L3](hereinafter, referred to as Compound Class SX₁₉₃₃).

A compound (L-1) wherein T represents a group represented by T33, R^{3b} represents a hydrogen atom, and R¹ represents any substituents indicated in [Table L1] to [Table L3] (hereinafter, referred to as Compound Class SX₁₉₃₄).

A compound (L-1) wherein T represents a group represented by T33, R^{3b} represents a chlorine atom, and R¹ represents any substituents indicated in [Table L1] to [Table L3] (hereinafter, referred to as Compound Class SX₁₉₃₅).

A compound (L-1) wherein T represents a group represented by T33, R^{3b} represents a cyclopropyl group, and R¹ represents any substituents indicated in [Table L1] to [Table L3] (hereinafter, referred to as Compound Class SX₁₉₃₆).

A compound (L-1) wherein T represents a group represented by T33, R^{3b} represents a 1-cyanocyclopropyl group, and R¹ represents any substituents indicated in [Table L1] to [Table L3] (hereinafter, referred to as Compound Class SX₁₉₃₇).

A compound (L-1) wherein T represents a group represented by T33, R^{3b} represents CF₃, and R¹ represents any substituents indicated in [Table L1] to [Table L3](hereinafter, referred to as Compound Class SX₁₉₃₈).

A compound (L-1) wherein T represents a group represented by T34, R^{3b} represents a hydrogen atom, and R¹ represents any substituents indicated in [Table L1] to [Table L3] (hereinafter, referred to as Compound Class SX₁₉₃₉).

A compound (L-1) wherein T represents a group represented by T34, R^{3b} represents a chlorine atom, and R¹ represents any substituents indicated in [Table L1] to [Table L3] (hereinafter, referred to as Compound Class SX₁₉₄₀).

A compound (L-1) wherein T represents a group represented by T34, R^{3b} represents a cyclopropyl group, and R¹ represents any substituents indicated in [Table L1] to [Table L3] (hereinafter, referred to as Compound Class SX₁₉₄₁).

A compound (L-1) wherein T represents a group represented by T34, R^{3b} represents a 1-cyanocyclopropyl group, and R¹ represents any substituents indicated in [Table L1] to [Table L3] (hereinafter, referred to as Compound Class SX₁₉₄₂).

A compound (L-1) wherein T represents a group represented by T34, R^{3b} represents CF₃, and R¹ represents any substituents indicated in [Table L1] to [Table L3](hereinafter, referred to as Compound Class SX₁₉₄₃).

A compound (L-1) wherein T represents a group represented by T35, R^{3b} represents a hydrogen atom, and R¹ represents any substituents indicated in [Table L1] to [Table L3] (hereinafter, referred to as Compound Class SX₁₉₄₄).

A compound (L-1) wherein T represents a group represented by T35, R^{3b} represents a chlorine atom, and R¹ represents any substituents indicated in [Table L1] to [Table L3] (hereinafter, referred to as Compound Class SX₁₉₄₅).

A compound (L-1) wherein T represents a group represented by T35, R^{3b} represents a cyclopropyl group, and R¹ represents any substituents indicated in [Table L1] to [Table L3] (hereinafter, referred to as Compound Class SX₁₉₄₆).

A compound (L-1) wherein T represents a group represented by T35, R^{3b} represents a 1-cyanocyclopropyl group, and R¹ represents any substituents indicated in [Table L1] to [Table L3] (hereinafter, referred to as Compound Class SX₁₉₄₇).

A compound (L-1) wherein T represents a group represented by T35, R^{3b} represents CF₃, and R¹ represents any substituents indicated in [Table L1] to [Table L3](hereinafter, referred to as Compound Class SX₁₉₄₈).

A compound (L-1) wherein T represents a group represented by T36, R^{3b} represents a hydrogen atom, and R¹ represents any substituents indicated in [Table L1] to [Table L3] (hereinafter, referred to as Compound Class SX₁₉₄₉).

A compound (L-1) wherein T represents a group represented by T36, R^{3b} represents a chlorine atom, and R¹ represents any substituents indicated in [Table L1] to [Table L3] (hereinafter, referred to as Compound Class SX₁₉₅₀).

A compound (L-1) wherein T represents a group represented by T36, R^{3b} represents a cyclopropyl group, and R¹ represents any substituents indicated in [Table L1] to [Table L3] (hereinafter, referred to as Compound Class SX₁₉₅₁).

A compound (L-1) wherein T represents a group represented by T36, R^{3b} represents a 1-cyanocyclopropyl group, and R¹ represents any substituents indicated in [Table L1] to [Table L3] (hereinafter, referred to as Compound Class SX₁₉₅₂).

A compound (L-1) wherein T represents a group represented by T36, R^{3b} represents CF₃, and R¹ represents any substituents indicated in [Table L1] to [Table L3](hereinafter, referred to as Compound Class SX₁₉₅₃).

A compound (L-2) wherein T represents a group represented by T32, R^{3b} represents a hydrogen atom, R^{3c} represents a hydrogen atom, and R¹ represents any substituents indicated in [Table L1] to [Table L3](hereinafter, referred to as Compound Class SX₁₉₅₄).

A compound (L-2) wherein T represents a group represented by T32, R^{3b} represents CF₃, R^{3c} represents a hydrogen atom, and R¹ represents any substituents indicated in [Table L1] to [Table L3] (hereinafter, referred to as Compound Class SX₁₉₅₅).

A compound (L-2) wherein T represents a group represented by T32, R^{3b} represents a chlorine atom, R^{3c} represents a hydrogen atom, and R¹ represents any substituents indicated in [Table L1] to [Table L3](hereinafter, referred to as Compound Class SX₁₉₅₆).

A compound (L-2) wherein T represents a group represented by T32, R^{3b} represents a bromine atom, R^{3c} represents a hydrogen atom, and R¹ represents any substituents indicated in [Table L1] to [Table L3](hereinafter, referred to as Compound Class SX₁₉₅₇).

A compound (L-2) wherein T represents a group represented by T32, R^{3b} represents an iodine atom, R^{3c} represents a hydrogen atom, and R¹ represents any substituents indicated in [Table L1] to [Table L3](hereinafter, referred to as Compound Class SX₁₉₅₈).

A compound (L-2) wherein T represents a group represented by T32, R^{3b} represents a hydrogen atom, R^{3c} represents CF₃, and R¹ represents any substituents indicated in [Table L1] to [Table L3] (hereinafter, referred to as Compound Class SX₁₉₅₉).

A compound (L-2) wherein T represents a group represented by T32, R^{3b} represents a hydrogen atom, R^{3c} represents a chlorine atom, and R¹ represents any substituents indicated in [Table L1] to [Table L3](hereinafter, referred to as Compound Class SX₁₉₆₀).

A compound (L-2) wherein T represents a group represented by T32, R^{3b} represents a hydrogen atom, R^{3c} represents a bromine atom, and R¹ represents any substituents indicated in [Table L1] to [Table L3] (hereinafter, referred to as Compound Class SX₁₉₆₁).

A compound (L-2) wherein T represents a group represented by T32, R^{3b} represents a hydrogen atom, R^{3c} represents an iodine atom, and R¹ represents any substituents indicated in [Table L1] to [Table L3] (hereinafter, referred to as Compound Class SX₁₉₆₂).

A compound (L-2) wherein T represents a group represented by T33, R^{3b} represents a hydrogen atom, R^{3c} represents a hydrogen atom, and R¹ represents any substituents indicated in [Table L1] to [Table L3](hereinafter, referred to as Compound Class SX₁₉₆₃).

A compound (L-2) wherein T represents a group represented by T33, R^{3b} represents CF₃, R^{3c} represents a hydrogen atom, and R¹ represents any substituents indicated in [Table L1] to [Table L3] (hereinafter, referred to as Compound Class SX₁₉₆₄).

A compound (L-2) wherein T represents a group represented by T33, R^{3b} represents a chlorine atom, R^{3c} represents a hydrogen atom, and R¹ represents any substituents indicated in [Table L1] to [Table L3](hereinafter, referred to as Compound Class SX₁₉₆₅).

A compound (L-2) wherein T represents a group represented by T33, R^{3b} represents a bromine atom, R^{3c} represents a hydrogen atom, and R¹ represents any substituents indicated in [Table L1] to [Table L3](hereinafter, referred to as Compound Class SX₁₉₆₆).

A compound (L-2) wherein T represents a group represented by T33, R^{3b} represents an iodine atom, R^{3c} represents a hydrogen atom, and R¹ represents any substituents indicated in [Table L1] to [Table L3](hereinafter, referred to as Compound Class SX₁₉₆₇).

A compound (L-2) wherein T represents a group represented by T33, R^{3b} represents a hydrogen atom, R^{3c} represents CF₃, and R¹ represents any substituents indicated in [Table L1] to [Table L3] (hereinafter, referred to as Compound Class SX₁₉₆₈).

A compound (L-2) wherein T represents a group represented by T33, R^{3b} represents a hydrogen atom, R^{3c} represents a chlorine atom, and R¹ represents any substituents indicated in [Table L1] to [Table L3](hereinafter, referred to as Compound Class SX₁₉₆₉).

A compound (L-2) wherein T represents a group represented by T33, R^{3b} represents a hydrogen atom, R^{3c} represents a bromine atom, and R¹ represents any substituents indicated in [Table L1] to [Table L3] (hereinafter, referred to as Compound Class SX₁₉₇₀).

A compound (L-2) wherein T represents a group represented by T33, R^{3b} represents a hydrogen atom, R^{3c} represents an iodine atom, and R¹ represents any substituents indicated in [Table L1] to [Table L3] (hereinafter, referred to as Compound Class SX₁₉₇₁).

A compound (L-2) wherein T represents a group represented by T34, R^{3b} represents a hydrogen atom, R^{3c} represents a hydrogen atom, and R¹ represents any substituents indicated in [Table L1] to [Table L3](hereinafter, referred to as Compound Class SX₁₉₇₂).

A compound (L-2) wherein T represents a group represented by T34, R^{3b} represents CF₃, R^{3c} represents a hydrogen atom, and R¹ represents any substituents indicated in [Table L1] to [Table L3] (hereinafter, referred to as Compound Class SX₁₉₇₃).

A compound (L-2) wherein T represents a group represented by T34, R^{3b} represents a chlorine atom, R^{3c} represents a hydrogen atom, and R¹ represents any substituents indicated in [Table L1] to [Table L3](hereinafter, referred to as Compound Class SX₁₉₇₄).

A compound (L-2) wherein T represents a group represented by T34, R^{3b} represents a bromine atom, R^{3c} represents a hydrogen atom, and R¹ represents any substituents indicated in [Table L1] to [Table L3](hereinafter, referred to as Compound Class SX₁₉₇₅).

A compound (L-2) wherein T represents a group represented by T34, R^{3b} represents an iodine atom, R^{3c} represents a hydrogen atom, and R¹ represents any substituents indicated in [Table L1] to [Table L3](hereinafter, referred to as Compound Class SX₁₉₇₆).

A compound (L-2) wherein T represents a group represented by T34, R^{3b} represents a hydrogen atom, R^{3c} represents CF₃, and R¹ represents any substituents indicated in [Table L1] to [Table L3] (hereinafter, referred to as Compound Class SX₁₉₇₇).

A compound (L-2) wherein T represents a group represented by T34, R^{3b} represents a hydrogen atom, R^{3c} represents a chlorine atom, and R¹ represents any substituents indicated in [Table L1] to [Table L3](hereinafter, referred to as Compound Class SX₁₉₇₈).

A compound (L-2) wherein T represents a group represented by T34, R^{3b} represents a hydrogen atom, R^{3c} represents a bromine atom, and R¹ represents any substituents indicated in [Table L1] to [Table L3] (hereinafter, referred to as Compound Class SX₁₉₇₉).

A compound (L-2) wherein T represents a group represented by T34, R^{3b} represents a hydrogen atom, R^{3c} represents an iodine atom, and R¹ represents any substituents indicated in [Table L1] to [Table L3] (hereinafter, referred to as Compound Class SX₁₉₈₀).

A compound (L-2) wherein T represents a group represented by T35, R^{3b} represents a hydrogen atom, R^{3c} represents a hydrogen atom, and R¹ represents any substituents indicated in [Table L1] to [Table L3](hereinafter, referred to as Compound Class SX₁₉₈₁).

A compound (L-2) wherein T represents a group represented by T35, R^{3b} represents CF₃, R^{3c} represents a hydrogen atom, and R¹ represents any substituents indicated in [Table L1] to [Table L3] (hereinafter, referred to as Compound Class SX₁₉₈₂).

A compound (L-2) wherein T represents a group represented by T35, R^{3b} represents a chlorine atom, R^{3c} represents a hydrogen atom, and R¹ represents any substituents indicated in [Table L1] to [Table L3](hereinafter, referred to as Compound Class SX₁₉₈₃).

A compound (L-2) wherein T represents a group represented by T35, R^{3b} represents a bromine atom, R^{3c} represents a hydrogen atom, and R¹ represents any substituents indicated in [Table L1] to [Table L3](hereinafter, referred to as Compound Class SX₁₉₈₄).

A compound (L-2) wherein T represents a group represented by T35, R^{3b} represents an iodine atom, R^{3c} represents a hydrogen atom, and R¹ represents any substituents indicated in [Table L1] to [Table L3](hereinafter, referred to as Compound Class SX₁₉₈₅).

A compound (L-2) wherein T represents a group represented by T35, R^{3b} represents a hydrogen atom, R^{3c} represents CF₃, and R¹ represents any substituents indicated in [Table L1] to [Table L3] (hereinafter, referred to as Compound Class SX₁₉₈₆).

A compound (L-2) wherein T represents a group represented by T35, R^{3b} represents a hydrogen atom, R^{3c} represents a chlorine atom, and R¹ represents any (hereinafter, referred to as Compound Class SX₁₉₈₇).

A compound (L-2) wherein T represents a group represented by T35, R^{3b} represents a hydrogen atom, R^{3c} represents a bromine atom, and R¹ represents any substituents indicated in [Table L1] to [Table L3] (hereinafter, referred to as Compound Class SX₁₉₈₈).

A compound (L-2) wherein T represents a group represented by T35, R^{3b} represents a hydrogen atom, R^{3c} represents an iodine atom, and R¹ represents any substituents indicated in [Table L1] to [Table L3] (hereinafter, referred to as Compound Class SX₁₉₈₉).

A compound (L-2) wherein T represents a group represented by T36, R^{3b} represents a hydrogen atom, R^{3c} represents a hydrogen atom, and R¹ represents any substituents indicated in [Table L1] to [Table L3](hereinafter, referred to as Compound Class SX₁₉₉₀).

A compound (L-2) wherein T represents a group represented by T36, R^{3b} represents CF₃, R^{3c} represents a hydrogen atom, and R¹ represents any substituents indicated in [Table L1] to [Table L3] (hereinafter, referred to as Compound Class SX₁₉₉₁).

A compound (L-2) wherein T represents a group represented by T36, R^{3b} represents a chlorine atom, R^{3c} represents a hydrogen atom, and R¹ represents any substituents indicated in [Table L1] to [Table L3](hereinafter, referred to as Compound Class SX₁₉₉₂).

A compound (L-2) wherein T represents a group represented by T36, R^{3b} represents a bromine atom, R^{3c} represents a hydrogen atom, and R¹ represents any substituents indicated in [Table L1] to [Table L3](hereinafter, referred to as Compound Class SX₁₉₉₃).

A compound (L-2) wherein T represents a group represented by T36, R^{3b} represents an iodine atom, R^{3c} represents a hydrogen atom, and R¹ represents any substituents indicated in [Table L1] to [Table L3](hereinafter, referred to as Compound Class SX₁₉₉₄).

A compound (L-2) wherein T represents a group represented by T36, R^{3b} represents a hydrogen atom, R^{3c} represents CF₃, and R¹ represents any substituents indicated in [Table L1] to [Table L3] (hereinafter, referred to as Compound Class SX₁₉₉₅).

A compound (L-2) wherein T represents a group represented by T36, R^{3b} represents a hydrogen atom, R^{3c} represents a chlorine atom, and R¹ represents any substituents indicated in [Table L1] to [Table L3](hereinafter, referred to as Compound Class SX₁₉₉₆).

A compound (L-2) wherein T represents a group represented by T36, R^{3b} represents a hydrogen atom, R^{3c} represents a bromine atom, and R¹ represents any substituents substituents indicated in [Table L1] to [Table L3] indicated in [Table L1] to [Table L3] (hereinafter, referred to as Compound Class SX₁₉₉₇).

A compound (L-2) wherein T represents a group represented by T36, R^{3b} represents a hydrogen atom, R^{3c} represents an iodine atom, and R¹ represents any substituents indicated in [Table L1] to [Table L3] (hereinafter, referred to as Compound Class SX₁₉₉₈).

A compound (L-3) wherein T represents a group represented by T32, R^{3b} represents a hydrogen atom, R^{3c} represents a hydrogen atom, and R¹ represents any substituents indicated in [Table L1] to [Table L3](hereinafter, referred to as Compound Class SX₁₉₉₉).

A compound (L-3) wherein T represents a group represented by T32, R^{3b} represents CF₃, R^{3c} represents a hydrogen atom, and R¹ represents any substituents indicated in [Table L1] to [Table L3] (hereinafter, referred to as Compound Class SX₂₀₀₀).

A compound (L-3) wherein T represents a group represented by T32, R^{3b} represents a chlorine atom, R^{3c} represents a hydrogen atom, and R¹ represents any substituents indicated in [Table L1] to [Table L3](hereinafter, referred to as Compound Class SX₂₀₀₁).

A compound (L-3) wherein T represents a group represented by T32, R^{3b} represents a bromine atom, R^{3c} represents a hydrogen atom, and R¹ represents any substituents indicated in [Table L1] to [Table L3](hereinafter, referred to as Compound Class SX₂₀₀₂).

A compound (L-3) wherein T represents a group represented by T32, R^{3b} represents an iodine atom, R^{3c} represents a hydrogen atom, and R¹ represents any substituents indicated in [Table L1] to [Table L3](hereinafter, referred to as Compound Class SX₂₀₀₃).

A compound (L-3) wherein T represents a group represented by T32, R^{3b} represents a hydrogen atom, R^{3c} represents CF₃, and R¹ represents any substituents indicated in [Table L1] to [Table L3] (hereinafter, referred to as Compound Class SX₂₀₀₄).

A compound (L-3) wherein T represents a group represented by T32, R^{3b} represents a hydrogen atom, R^{3c} represents a chlorine atom, and R¹ represents any substituents indicated in [Table L1] to [Table L3](hereinafter, referred to as Compound Class SX₂₀₀₅).

A compound (L-3) wherein T represents a group represented by T32, R^{3b} represents a hydrogen atom, R^{3c} represents a bromine atom, and R¹ represents any substituents indicated in [Table L1] to [Table L3] (hereinafter, referred to as Compound Class SX₂₀₀₆).

A compound (L-3) wherein T represents a group represented by T32, R^{3b} represents a hydrogen atom, R^{3c} represents an iodine atom, and R¹ represents any substituents indicated in [Table L1] to [Table L3] (hereinafter, referred to as Compound Class SX₂₀₀₇).

A compound (L-3) wherein T represents a group represented by T33, R^{3b} represents a hydrogen atom, R^{3c} represents a hydrogen atom, and R¹ represents any substituents indicated in [Table L1] to [Table L3](hereinafter, referred to as Compound Class SX₂₀₀₈).

A compound (L-3) wherein T represents a group represented by T33, R^{3b} represents CF₃, R^{3c} represents a hydrogen atom, and R¹ represents any substituents indicated in [Table L1] to [Table L3] (hereinafter, referred to as Compound Class SX₂₀₀₉).

A compound (L-3) wherein T represents a group represented by T33, R^{3b} represents a chlorine atom, R^{3c} represents a hydrogen atom, and R¹ represents any substituents indicated in [Table L1] to [Table L3](hereinafter, referred to as Compound Class SX₂₀₁₀).

A compound (L-3) wherein T represents a group represented by T33, R^{3b} represents a bromine atom, R^{3c} represents a hydrogen atom, and R¹ represents any substituents indicated in [Table L1] to [Table L3](hereinafter, referred to as Compound Class SX₂₀₁₁).

A compound (L-3) wherein T represents a group represented by T33, R^{3b} represents an iodine atom, R^{3c} represents a hydrogen atom, and R¹ represents any substituents indicated in [Table L1] to [Table L3](hereinafter, referred to as Compound Class SX₂₀₁₂).

A compound (L-3) wherein T represents a group represented by T33, R^{3b} represents a hydrogen atom, R^{3c} represents CF₃, and R¹ represents any substituents indicated in [Table L1] to [Table L3] (hereinafter, referred to as Compound Class SX₂₀₁₃).

A compound (L-3) wherein T represents a group represented by T33, R^{3b} represents a hydrogen atom, R^{3c} represents a chlorine atom, and R¹ represents any substituents indicated in [Table L1] to [Table L3](hereinafter, referred to as Compound Class SX₂₀₁₄).

A compound (L-3) wherein T represents a group represented by T33, R^{3b} represents a hydrogen atom, R^{3c} represents a bromine atom, and R¹ represents any substituents indicated in [Table L1] to [Table L3] (hereinafter, referred to as Compound Class SX₂₀₁₅).

A compound (L-3) wherein T represents a group represented by T33, R^{3b} represents a hydrogen atom, R^{3c} represents an iodine atom, and R¹ represents any substituents indicated in [Table L1] to [Table L3] (hereinafter, referred to as Compound Class SX₂₀₁₆).

A compound (L-3) wherein T represents a group represented by T34, R^{3b} represents a hydrogen atom, R^{3c} represents a hydrogen atom, and R¹ represents any substituents indicated in [Table L1] to [Table L3](hereinafter, referred to as Compound Class SX₂₀₁₇).

A compound (L-3) wherein T represents a group represented by T34, R^{3b} represents CF₃, R^{3c} represents a hydrogen atom, and R¹ represents any substituents indicated in [Table L1] to [Table L3] (hereinafter, referred to as Compound Class SX₂₀₁₈).

A compound (L-3) wherein T represents a group represented by T34, R^{3b} represents a chlorine atom, R^{3c} represents a hydrogen atom, and R¹ represents any substituents indicated in [Table L1] to [Table L3](hereinafter, referred to as Compound Class SX₂₀₁₉).

A compound (L-3) wherein T represents a group represented by T34, R^{3b} represents a bromine atom, R^{3c} represents a hydrogen atom, and R¹ represents any substituents indicated in [Table L1] to [Table L3](hereinafter, referred to as Compound Class SX₂ₒ₂₀).

A compound (L-3) wherein T represents a group represented by T34, R^{3b} represents an iodine atom, R^{3c} represents a hydrogen atom, and R¹ represents any substituents indicated in [Table L1] to [Table L3](hereinafter, referred to as Compound Class SX₂₀₂₁).

A compound (L-3) wherein T represents a group represented by T34, R^{3b} represents a hydrogen atom, R^{3c} represents CF₃, and R¹ represents any substituents indicated in [Table L1] to [Table L3] (hereinafter, referred to as Compound Class SX₂₀₂₂).

A compound (L-3) wherein T represents a group represented by T34, R^{3b} represents a hydrogen atom, R^{3c} represents a chlorine atom, and R¹ represents any substituents indicated in [Table L1] to [Table L3](hereinafter, referred to as Compound Class SX₂₀₂₃).

A compound (L-3) wherein T represents a group represented by T34, R^{3b} represents a hydrogen atom, R^{3c} represents a bromine atom, and R¹ represents any substituents indicated in [Table L1] to [Table L3] (hereinafter, referred to as Compound Class SX₂₀₂₄).

A compound (L-3) wherein T represents a group represented by T34, R^{3b} represents a hydrogen atom, R^{3c} represents an iodine atom, and R¹ represents any substituents indicated in [Table L1] to [Table L3] (hereinafter, referred to as Compound Class SX₂₀₂₅).

A compound (L-3) wherein T represents a group represented by T35, R^{3b} represents a hydrogen atom, R^{3c} represents a hydrogen atom, and R¹ represents any substituents indicated in [Table L1] to [Table L3](hereinafter, referred to as Compound Class SX₂₀₂₆).

A compound (L-3) wherein T represents a group represented by T35, R^{3b} represents CF₃, R^{3c} represents a hydrogen atom, and R¹ represents any substituents indicated in [Table L1] to [Table L3] (hereinafter, referred to as Compound Class SX₂₀₂₇).

A compound (L-3) wherein T represents a group represented by T35, R^{3b} represents a chlorine atom, R^{3c} represents a hydrogen atom, and R¹ represents any substituents indicated in [Table L1] to [Table L3](hereinafter, referred to as Compound Class SX₂₀₂₈).

A compound (L-3) wherein T represents a group represented by T35, R^{3b} represents a bromine atom, R^{3c} represents a hydrogen atom, and R¹ represents any substituents indicated in [Table L1] to [Table L3](hereinafter, referred to as Compound Class SX₂₀₂₉).

A compound (L-3) wherein T represents a group represented by T35, R^{3b} represents an iodine atom, R^{3c} represents a hydrogen atom, and R¹ represents any substituents indicated in [Table L1] to [Table L3](hereinafter, referred to as Compound Class SX₂₀₃₀).

A compound (L-3) wherein T represents a group represented by T35, R^{3b} represents a hydrogen atom, R^{3c} represents CF₃, and R¹ represents any substituents indicated in [Table L1] to [Table L3] (hereinafter, referred to as Compound Class SX₂₀₃₁).

A compound (L-3) wherein T represents a group represented by T35, R^{3b} represents a hydrogen atom, R^{3c} represents a chlorine atom, and R¹ represents any substituents indicated in [Table L1] to [Table L3](hereinafter, referred to as Compound Class SX₂₀₃₂).

A compound (L-3) wherein T represents a group represented by T35, R^{3b} represents a hydrogen atom, R^{3c} represents a bromine atom, and R¹ represents any substituents indicated in [Table L1] to [Table L3] (hereinafter, referred to as Compound Class SX₂₀₃₃).

A compound (L-3) wherein T represents a group represented by T35, R^{3b} represents a hydrogen atom, R^{3c} represents an iodine atom, and R¹ represents any substituents indicated in [Table L1] to [Table L3] (hereinafter, referred to as Compound Class SX₂₀₃₄).

A compound (L-3) wherein T represents a group represented by T36, R^{3b} represents a hydrogen atom, R^{3c} represents a hydrogen atom, and R¹ represents any substituents indicated in [Table L1] to [Table L3](hereinafter, referred to as Compound Class SX₂₀₃₅).

A compound (L-3) wherein T represents a group represented by T36, R^{3b} represents CF₃, R^{3c} represents a hydrogen atom, and R¹ represents any substituents indicated in [Table L1] to [Table L3] (hereinafter, referred to as Compound Class SX₂₀₃₆).

A compound (L-3) wherein T represents a group represented by T36, R^{3b} represents a chlorine atom, R^{3c} represents a hydrogen atom, and R¹ represents any substituents indicated in [Table L1] to [Table L3](hereinafter, referred to as Compound Class SX₂₀₃₇).

A compound (L-3) wherein T represents a group represented by T36, R^{3b} represents a bromine atom, R^{3c} represents a hydrogen atom, and R¹ represents any substituents indicated in [Table L1] to [Table L3](hereinafter, referred to as Compound Class SX₂₀₃₈).

A compound (L-3) wherein T represents a group represented by T36, R^{3b} represents an iodine atom, R^{3c} represents a hydrogen atom, and R¹ represents any substituents indicated in [Table L1] to [Table L3](hereinafter, referred to as Compound Class SX₂₀₃₉).

A compound (L-3) wherein T represents a group represented by T36, R^{3b} represents a hydrogen atom, R^{3c} represents CF₃, and R¹ represents any substituents indicated in [Table L1] to [Table L3] (hereinafter, referred to as Compound Class SX₂ₒ₄₀).

A compound (L-3) wherein T represents a group represented by T36, R^{3b} represents a hydrogen atom, R^{3c} represents a chlorine atom, and R¹ represents any substituents indicated in [Table L1] to [Table L3](hereinafter, referred to as Compound Class SX₂₀₄₁).

A compound (L-3) wherein T represents a group represented by T36, R^{3b} represents a hydrogen atom, R^{3c} represents a bromine atom, and R¹ represents any substituents indicated in [Table L1] to [Table L3] (hereinafter, referred to as Compound Class SX₂₀₄₂).

A compound (L-3) wherein T represents a group represented by T36, R^{3b} represents a hydrogen atom, R^{3c} represents an iodine atom, and R¹ represents any substituents indicated in [Table L1] to [Table L3] (hereinafter, referred to as Compound Class SX₂₀₄₃).

A compound (L-4) wherein T represents a group represented by T32, R^{3b} represents a hydrogen atom, R^{3c} represents a hydrogen atom, and R¹ represents any substituents indicated in [Table L1] to [Table L3](hereinafter, referred to as Compound Class SX₂₀₄₅).

A compound (L-4) wherein T represents a group represented by T32, R^{3b} represents CF₃, R^{3c} represents a hydrogen atom, and R¹ represents any substituents indicated in [Table L1] to [Table L3] (hereinafter, referred to as Compound Class SX₂₀₄₆).

A compound (L-4) wherein T represents a group represented by T32, R^{3b} represents a chlorine atom, R^{3c} represents a hydrogen atom, and R¹ represents any substituents indicated in [Table L1] to [Table L3](hereinafter, referred to as Compound Class SX₂₀₄₇).

A compound (L-4) wherein T represents a group represented by T32, R^{3b} represents a bromine atom, R^{3c} represents a hydrogen atom, and R¹ represents any substituents indicated in [Table L1] to [Table L3](hereinafter, referred to as Compound Class SX₂₀₄₈).

A compound (L-4) wherein T represents a group represented by T32, R^{3b} represents an iodine atom, R^{3c} represents a hydrogen atom, and R¹ represents any substituents indicated in [Table L1] to [Table L3](hereinafter, referred to as Compound Class SX₂₀₄₉).

A compound (L-4) wherein T represents a group represented by T32, R^{3b} represents a hydrogen atom, R^{3c} represents CF₃, and R¹ represents any substituents indicated in [Table L1] to [Table L3] (hereinafter, referred to as Compound Class SX₂₀₅₀).

A compound (L-4) wherein T represents a group represented by T32, R^{3b} represents a hydrogen atom, R^{3c} represents a chlorine atom, and R¹ represents any substituents indicated in [Table L1] to [Table L3](hereinafter, referred to as Compound Class SX₂₀₅₁).

A compound (L-4) wherein T represents a group represented by T32, R^{3b} represents a hydrogen atom, R^{3c} represents a bromine atom, and R¹ represents any substituents indicated in [Table L1] to [Table L3] (hereinafter, referred to as Compound Class SX₂₀₅₂).

A compound (L-3) wherein T represents a group represented by T32, R^{3b} represents a hydrogen atom, R^{3c} represents an iodine atom, and R¹ represents any substituents indicated in [Table L1] to [Table L3] (hereinafter, referred to as Compound Class SX₂₀₅₃).

A compound (L-4) wherein T represents a group represented by T33, R^{3b} represents a hydrogen atom, R^{3c} represents a hydrogen atom, and R¹ represents any substituents indicated in [Table L1] to [Table L3](hereinafter, referred to as Compound Class SX₂₀₅₄).

A compound (L-4) wherein T represents a group represented by T33, R^{3b} represents CF₃, R^{3c} represents a hydrogen atom, and R¹ represents any substituents indicated in [Table L1] to [Table L3] (hereinafter, referred to as Compound Class SX₂₀₅₅).

A compound (L-4) wherein T represents a group represented by T33, R^{3b} represents a chlorine atom, R^{3c} represents a hydrogen atom, and R¹ represents any substituents indicated in [Table L1] to [Table L3](hereinafter, referred to as Compound Class SX₂₀₅₆).

A compound (L-4) wherein T represents a group represented by T33, R^{3b} represents a bromine atom, R^{3c} represents a hydrogen atom, and R¹ represents any substituents indicated in [Table L1] to [Table L3](hereinafter, referred to as Compound Class SX₂₀₅₇).

A compound (L-4) wherein T represents a group represented by T33, R^{3b} represents an iodine atom, R^{3c} represents a hydrogen atom, and R¹ represents any substituents indicated in [Table L1] to [Table L3](hereinafter, referred to as Compound Class SX₂₀₅₈).

A compound (L-4) wherein T represents a group represented by T33, R^{3b} represents a hydrogen atom, R^{3c} represents CF₃, and R¹ represents any substituents indicated in [Table L1] to [Table L3] (hereinafter, referred to as Compound Class SX₂₀₅₉).

A compound (L-4) wherein T represents a group represented by T33, R^{3b} represents a hydrogen atom, R^{3c} represents a chlorine atom, and R¹ represents any substituents indicated in [Table L1] to [Table L3](hereinafter, referred to as Compound Class SX₂₀₆₀).

A compound (L-4) wherein T represents a group represented by T33, R^{3b} represents a hydrogen atom, R^{3c} represents a bromine atom, and R¹ represents any substituents indicated in [Table L1] to [Table L3] (hereinafter, referred to as Compound Class SX₂₀₆₁).

A compound (L-3) wherein T represents a group represented by T33, R^{3b} represents a hydrogen atom, R^{3c} represents an iodine atom, and R¹ represents any substituents indicated in [Table L1] to [Table L3] (hereinafter, referred to as Compound Class SX₂₀₆₂).

A compound (L-4) wherein T represents a group represented by T34, R^{3b} represents a hydrogen atom, R^{3c} represents a hydrogen atom, and R¹ represents any substituents indicated in [Table L1] to [Table L3](hereinafter, referred to as Compound Class SX₂₀₆₃).

A compound (L-4) wherein T represents a group represented by T34, R^{3b} represents CF₃, R^{3c} represents a hydrogen atom, and R¹ represents any substituents indicated in [Table L1] to [Table L3] (hereinafter, referred to as Compound Class SX₂₀₆₄).

A compound (L-4) wherein T represents a group represented by T34, R^{3b} represents a chlorine atom, R^{3c} represents a hydrogen atom, and R¹ represents any substituents indicated in [Table L1] to [Table L3](hereinafter, referred to as Compound Class SX₂₀₆₅).

A compound (L-4) wherein T represents a group represented by T34, R^{3b} represents a bromine atom, R^{3c} represents a hydrogen atom, and R¹ represents any substituents indicated in [Table L1] to [Table L3](hereinafter, referred to as Compound Class SX₂₀₆₆).

A compound (L-4) wherein T represents a group represented by T34, R^{3b} represents an iodine atom, R^{3c} represents a hydrogen atom, and R¹ represents any substituents indicated in [Table L1] to [Table L3](hereinafter, referred to as Compound Class SX₂₀₆₇).

A compound (L-4) wherein T represents a group represented by T34, R^{3b} represents a hydrogen atom, R^{3c} represents CF₃, and R¹ represents any substituents indicated in [Table L1] to [Table L3] (hereinafter, referred to as Compound Class SX₂₀₆₈).

A compound (L-4) wherein T represents a group represented by T34, R^{3b} represents a hydrogen atom, R^{3c} represents a chlorine atom, and R¹ represents any substituents indicated in [Table L1] to [Table L3](hereinafter, referred to as Compound Class SX₂₀₆₉).

A compound (L-4) wherein T represents a group represented by T34, R^{3b} represents a hydrogen atom, R^{3c} represents a bromine atom, and R¹ represents any substituents indicated in [Table L1] to [Table L3] (hereinafter, referred to as Compound Class SX₂₀₇₀).

A compound (L-3) wherein T represents a group represented by T34, R^{3b} represents a hydrogen atom, R^{3c} represents an iodine atom, and R¹ represents any substituents indicated in [Table L1] to [Table L3] (hereinafter, referred to as Compound Class SX₂₀₇₁).

A compound (L-4) wherein T represents a group represented by T35, R^{3b} represents a hydrogen atom, R^{3c} represents a hydrogen atom, and R¹ represents any substituents indicated in [Table L1] to [Table L3](hereinafter, referred to as Compound Class SX₂₀₇₂).

A compound (L-4) wherein T represents a group represented by T35, R^{3b} represents CF₃, R^{3c} represents a hydrogen atom, and R¹ represents any substituents indicated in [Table L1] to [Table L3] (hereinafter, referred to as Compound Class SX₂₀₇₃).

A compound (L-4) wherein T represents a group represented by T35, R^{3b} represents a chlorine atom, R^{3c} represents a hydrogen atom, and R¹ represents any substituents indicated in [Table L1] to [Table L3](hereinafter, referred to as Compound Class SX₂₀₇₄).

A compound (L-4) wherein T represents a group represented by T35, R^{3b} represents a bromine atom, R^{3c} represents a hydrogen atom, and R¹ represents any substituents indicated in [Table L1] to [Table L3](hereinafter, referred to as Compound Class SX₂₀₇₅).

A compound (L-4) wherein T represents a group represented by T35, R^{3b} represents an iodine atom, R^{3c} represents a hydrogen atom, and R¹ represents any substituents indicated in [Table L1] to [Table L3](hereinafter, referred to as Compound Class SX₂₀₇₆).

A compound (L-4) wherein T represents a group represented by T35, R^{3b} represents a hydrogen atom, R^{3c} represents CF₃, and R¹ represents any substituents indicated in [Table L1] to [Table L3] (hereinafter, referred to as Compound Class SX₂₀₇₇).

A compound (L-4) wherein T represents a group represented by T35, R^{3b} represents a hydrogen atom, R^{3c} represents a chlorine atom, and R¹ represents any substituents indicated in [Table L1] to [Table L3](hereinafter, referred to as Compound Class SX₂₀₇₈).

A compound (L-4) wherein T represents a group represented by T35, R^{3b} represents a hydrogen atom, R^{3c} represents a bromine atom, and R¹ represents any substituents indicated in [Table L1] to [Table L3] (hereinafter, referred to as Compound Class SX₂₀₇₉).

A compound (L-3) wherein T represents a group represented by T35, R^{3b} represents a hydrogen atom, R^{3c} represents an iodine atom, and R¹ represents any substituents indicated in [Table L1] to [Table L3] (hereinafter, referred to as Compound Class SX₂₀₈₀).

A compound (L-4) wherein T represents a group represented by T36, R^{3b} represents a hydrogen atom, R^{3c} represents a hydrogen atom, and R¹ represents any substituents indicated in [Table L1] to [Table L3](hereinafter, referred to as Compound Class SX₂₀₈₁).

A compound (L-4) wherein T represents a group represented by T36, R^{3b} represents CF₃, R^{3c} represents a hydrogen atom, and R¹ represents any substituents indicated in [Table L1] to [Table L3] (hereinafter, referred to as Compound Class SX₂₀₈₂).

A compound (L-4) wherein T represents a group represented by T36, R^{3b} represents a chlorine atom, R^{3c} represents a hydrogen atom, and R¹ represents any substituents indicated in [Table L1] to [Table L3](hereinafter, referred to as Compound Class SX₂₀₈₃).

A compound (L-4) wherein T represents a group represented by T36, R^{3b} represents a bromine atom, R^{3c} represents a hydrogen atom, and R¹ represents any substituents indicated in [Table L1] to [Table L3](hereinafter, referred to as Compound Class SX₂₀₈₄).

A compound (L-4) wherein T represents a group represented by T36, R^{3b} represents an iodine atom, R^{3c} represents a hydrogen atom, and R¹ represents any substituents indicated in [Table L1] to [Table L3](hereinafter, referred to as Compound Class SX₂₀₈₅).

A compound (L-4) wherein T represents a group represented by T36, R^{3b} represents a hydrogen atom, R^{3c} represents CF₃, and R¹ represents any substituents indicated in [Table L1] to [Table L3] (hereinafter, referred to as Compound Class SX₂₀₈₆).

A compound (L-4) wherein T represents a group represented by T36, R^{3b} represents a hydrogen atom, R^{3c} represents a chlorine atom, and R¹ represents any substituents indicated in [Table L1] to [Table L3](hereinafter, referred to as Compound Class SX₂₀₈₇).

A compound (L-4) wherein T represents a group represented by T36, R^{3b} represents a hydrogen atom, R^{3c} represents a bromine atom, and R¹ represents any substituents indicated in [Table L1] to [Table L3] (hereinafter, referred to as Compound Class SX₂₀₈₈).

A compound (L-3) wherein T represents a group represented by T36, R^{3b} represents a hydrogen atom, R^{3c} represents an iodine atom, and R¹ represents any substituents indicated in [Table L1] to [Table L3] (hereinafter, referred to as Compound Class SX₂₀₈₉).

A compound (L-5) wherein T represents a group represented by T32, R^{3c} represents a hydrogen atom, and R¹ represents any substituents indicated in [Table L1] to [Table L3] (hereinafter, referred to as Compound Class SX₂₀₉₀).

A compound (L-5) wherein T represents a group represented by T32, R^{3c} represents CF₃, and R¹ represents any substituents indicated in [Table L1] to [Table L3](hereinafter, referred to as Compound Class SX₂₀₉₁).

A compound (L-5) wherein T represents a group represented by T32, R^{3c} represents a chlorine atom, and R¹ represents any substituents indicated in [Table L1] to [Table L3] (hereinafter, referred to as Compound Class SX₂₀₉₂).

A compound (L-5) wherein T represents a group represented by T32, R^{3c} represents a bromine atom, and R¹ represents any substituents indicated in [Table L1] to [Table L3] (hereinafter, referred to as Compound Class SX₂₀₉₃).

A compound (L-5) wherein T represents a group represented by T32, R^{3c} represents an iodine atom, R^{3c} represents a hydrogen atom, and R¹ represents any substituents indicated in [Table L1] to [Table L3](hereinafter, referred to as Compound Class SX₂₀₉₅).

A compound (L-5) wherein T represents a group represented by T33, R^{3c} represents a hydrogen atom, and R¹ represents any substituents indicated in [Table L1] to [Table L3] (hereinafter, referred to as Compound Class SX₂₀₉₅).

A compound (L-5) wherein T represents a group represented by T33, R^{3c} represents CF₃, and R¹ represents any substituents indicated in [Table L1] to [Table L3](hereinafter, referred to as Compound Class SX₂₀₉₆).

A compound (L-5) wherein T represents a group represented by T33, R^{3c} represents a chlorine atom, and R¹ represents any substituents indicated in [Table L1] to [Table L3] (hereinafter, referred to as Compound Class SX₂₀₉₇).

A compound (L-5) wherein T represents a group represented by T33, R^{3c} represents a bromine atom, and R¹ represents any substituents indicated in [Table L1] to [Table L3] (hereinafter, referred to as Compound Class SX₂₀₉₈).

A compound (L-5) wherein T represents a group represented by T33, R^{3c} represents an iodine atom, R^{3c} represents a hydrogen atom, and R¹ represents any (hereinafter, referred to as Compound Class SX₂₀₉₉).

A compound (L-5) wherein T represents a group represented by T34, R^{3c} represents a hydrogen atom, and R¹ represents any substituents indicated in [Table L1] to [Table L3] (hereinafter, referred to as Compound Class SX₂₁₀₀).

A compound (L-5) wherein T represents a group represented by T34, R^{3c} represents CF₃, and R¹ represents any substituents indicated in [Table L1] to [Table L3](hereinafter, referred to as Compound Class SX₂₁₀₁).

A compound (L-5) wherein T represents a group represented by T34, R^{3c} represents a chlorine atom, and R¹ represents any substituents indicated in [Table L1] to [Table L3] (hereinafter, referred to as Compound Class SX₂₁₀₂).

A compound (L-5) wherein T represents a group represented by T34, R^{3c} represents a bromine atom, and R¹ represents any substituents indicated in [Table L1] to [Table L3] (hereinafter, referred to as Compound Class SX₂₁₀₃).

A compound (L-5) wherein T represents a group represented by T34, R^{3c} represents an iodine atom, R^{3c} represents a hydrogen atom, and R¹ represents any substituents indicated in [Table L1] to [Table L3](hereinafter, referred to as Compound Class SX₂₁₀₄).

A compound (L-5) wherein T represents a group represented by T35, R^{3c} represents a hydrogen atom, and R¹ represents any substituents indicated in [Table L1] to [Table L3] (hereinafter, referred to as Compound Class SX₂₁₀₅).

A compound (L-5) wherein T represents a group represented by T35, R^{3c} represents CF₃, and R¹ represents any substituents indicated in [Table L1] to [Table L3](hereinafter, referred to as Compound Class SX₂₁₀₆).

A compound (L-5) wherein T represents a group represented by T35, R^{3c} represents a chlorine atom, and R¹ represents any substituents indicated in [Table L1] to [Table L3] (hereinafter, referred to as Compound Class SX₂₁₀₇).

A compound (L-5) wherein T represents a group represented by T35, R^{3c} represents a bromine atom, and R¹ represents any substituents indicated in [Table L1] to [Table L3] (hereinafter, referred to as Compound Class SX₂₁₀₈).

A compound (L-5) wherein T represents a group represented by T35, R^{3c} represents an iodine atom, R^{3c} represents a hydrogen atom, and R¹ represents any substituents indicated in [Table L1] to [Table L3](hereinafter, referred to as Compound Class SX₂₁₀₉).

A compound (L-5) wherein T represents a group represented by T36, R^{3c} represents a hydrogen atom, and R¹ represents any substituents indicated in [Table L1] to [Table L3] (hereinafter, referred to as Compound Class SX₂₁₁₀).

A compound (L-5) wherein T represents a group represented by T36, R^{3c} represents CF₃, and R¹ represents any substituents indicated in [Table L1] to [Table L3](hereinafter, referred to as Compound Class SX₂₁₁₁).

A compound (L-5) wherein T represents a group represented by T36, R^{3c} represents a chlorine atom, and R¹ represents any substituents indicated in [Table L1] to [Table L3] (hereinafter, referred to as Compound Class SX₂₁₁₂).

A compound (L-5) wherein T represents a group represented by T36, R^{3c} represents a bromine atom, and R¹ represents any substituents indicated in [Table L1] to [Table L3] (hereinafter, referred to as Compound Class SX₂₁₁₃).

A compound (L-5) wherein T represents a group represented by T36, R^{3c} represents an iodine atom, R^{3c} represents a hydrogen atom, and R¹ represents any substituents indicated in [Table L1] to [Table L3](hereinafter, referred to as Compound Class SX₂₁₁₄).

A compound (L-6) wherein T represents a group represented by T32, R^{3b} represents a hydrogen atom, and R¹ represents any substituents indicated in [Table L1] to [Table L3] (hereinafter, referred to as Compound Class SX₂₁₁₅).

A compound (L-6) wherein T represents a group represented by T32, R^{3b} represents CF₃, and R¹ represents any substituents indicated in [Table L1] to [Table L3](hereinafter, referred to as Compound Class SX₂₁₁₆).

A compound (L-6) wherein T represents a group represented by T32, R^{3b} represents a chlorine atom, and R¹ represents any substituents indicated in [Table L1] to [Table L3] (hereinafter, referred to as Compound Class SX₂₁₁₇).

A compound (L-6) wherein T represents a group represented by T32, R^{3b} represents a bromine atom, and R¹ represents any substituents indicated in [Table L1] to [Table L3] (hereinafter, referred to as Compound Class SX₂₁₁₈).

A compound (L-6) wherein T represents a group represented by T32, R^{3b} represents an iodine atom, R^{3c} represents a hydrogen atom, and R¹ represents any substituents indicated in [Table L1] to [Table L3](hereinafter, referred to as Compound Class SX₂₁₁₉).

A compound (L-6) wherein T represents a group represented by T33, R^{3b} represents a hydrogen atom, and R¹ represents any substituents indicated in [Table L1] to [Table L3] (hereinafter, referred to as Compound Class SX₂₁₂₀).

A compound (L-6) wherein T represents a group represented by T33, R^{3b} represents CF₃, and R¹ represents any substituents indicated in [Table L1] to [Table L3](hereinafter, referred to as Compound Class SX₂₁₂₁).

A compound (L-6) wherein T represents a group represented by T33, R^{3b} represents a chlorine atom, and R¹ represents any substituents indicated in [Table L1] to [Table L3] (hereinafter, referred to as Compound Class SX₂₁₂₂).

A compound (L-6) wherein T represents a group represented by T33, R^{3b} represents a bromine atom, and R¹ represents any substituents indicated in [Table L1] to [Table L3] (hereinafter, referred to as Compound Class SX₂₁₂₃).

A compound (L-6) wherein T represents a group represented by T33, R^{3b} represents an iodine atom, R^{3c} represents a hydrogen atom, and R¹ represents any substituents indicated in [Table L1] to [Table L3](hereinafter, referred to as Compound Class SX₂₁₂₄).

A compound (L-6) wherein T represents a group represented by T34, R^{3b} represents a hydrogen atom, and R¹ represents any substituents indicated in [Table L1] to [Table L3] (hereinafter, referred to as Compound Class SX₂₁₂₅).

A compound (L-6) wherein T represents a group represented by T34, R^{3b} represents CF₃, and R¹ represents any substituents indicated in [Table L1] to [Table L3](hereinafter, referred to as Compound Class SX₂₁₂₆).

A compound (L-6) wherein T represents a group represented by T34, R^{3b} represents a chlorine atom, and R¹ represents any substituents indicated in [Table L1] to [Table L3] (hereinafter, referred to as Compound Class SX₂₁₂₇).

A compound (L-6) wherein T represents a group represented by T34, R^{3b} represents a bromine atom, and R¹ represents any substituents indicated in [Table L1] to [Table L3] (hereinafter, referred to as Compound Class SX₂₁₂₈).

A compound (L-6) wherein T represents a group represented by T34, R^{3b} represents an iodine atom, R^{3c} represents a hydrogen atom, and R¹ represents any substituents indicated in [Table L1] to [Table L3](hereinafter, referred to as Compound Class SX₂₁₂₉).

A compound (L-6) wherein T represents a group represented by T35, R^{3b} represents a hydrogen atom, and R¹ represents any substituents indicated in [Table L1] to [Table L3] (hereinafter, referred to as Compound Class SX₂₁₃₀).

A compound (L-6) wherein T represents a group represented by T35, R^{3b} represents CF₃, and R¹ represents any substituents indicated in [Table L1] to [Table L3](hereinafter, referred to as Compound Class SX₂₁₃₁).

A compound (L-6) wherein T represents a group represented by T35, R^{3b} represents a chlorine atom, and R¹ represents any substituents indicated in [Table L1] to [Table L3] (hereinafter, referred to as Compound Class SX₂₁₃₂).

A compound (L-6) wherein T represents a group represented by T35, R^{3b} represents a bromine atom, and R¹ represents any substituents indicated in [Table L1] to [Table L3] (hereinafter, referred to as Compound Class SX₂₁₃₃).

A compound (L-6) wherein T represents a group represented by T35, R^{3b} represents an iodine atom, R^{3c} represents a hydrogen atom, and R¹ represents any substituents indicated in [Table L1] to [Table L3](hereinafter, referred to as Compound Class SX₂₁₃₄).

A compound (L-6) wherein T represents a group represented by T36, R^{3b} represents a hydrogen atom, and R¹ represents any substituents indicated in [Table L1] to [Table L3] (hereinafter, referred to as Compound Class SX₂₁₃₅).

A compound (L-6) wherein T represents a group represented by T36, R^{3b} represents CF₃, and R¹ represents any substituents indicated in [Table L1] to [Table L3](hereinafter, referred to as Compound Class SX₂₁₃₆).

A compound (L-6) wherein T represents a group represented by T36, R^{3b} represents a chlorine atom, and R¹ represents any substituents indicated in [Table L1] to [Table L3] (hereinafter, referred to as Compound Class SX₂₁₃₇).

A compound (L-6) wherein T represents a group represented by T36, R^{3b} represents a bromine atom, and R¹ represents any substituents indicated in [Table L1] to [Table L3] (hereinafter, referred to as Compound Class SX₂₁₃₈).

A compound (L-6) wherein T represents a group represented by T36, R^{3b} represents an iodine atom, R^{3c} represents a hydrogen atom, and R¹ represents any substituents indicated in [Table L1] to [Table L3](hereinafter, referred to as Compound Class SX₂₁₃₉).

A compound (L-1) wherein T represents a group represented by T32, R¹ represents CF₃, and R^{3b} represents any substituents indicated in [Table L4] to [Table L12](hereinafter, referred to as Compound Class SX₂₁₄₀).

A compound (L-1) wherein T represents a group represented by T32, R¹ represents C₂F5, and R^{3b} represents any substituents indicated in [Table L4] to [Table L12](hereinafter, referred to as Compound Class SX₂₁₄₁).

A compound (L-1) wherein T represents a group represented by T32, R¹ represents OCF₃, and R^{3b} represents any substituents indicated in [Table L4] to [Table L12](hereinafter, referred to as Compound Class SX₂₁₄₂).

A compound (L-1) wherein T represents a group represented by T32, R¹ represents SCF₃, and R^{3b} represents any substituents indicated in [Table L4] to [Table L12](hereinafter, referred to as Compound Class SX₂₁₄₃).

A compound (L-1) wherein T represents a group represented by T32, R¹ represents S(O)CF₃, and R^{3b} represents any substituents indicated in [Table L4] to [Table L12](hereinafter, referred to as Compound Class SX₂₁₄₄).

A compound (L-1) wherein T represents a group represented by T32, R¹ represents S(O)₂CF₃, and R^{3b} represents any substituents indicated in [Table L4] to [Table L12](hereinafter, referred to as Compound Class SX₂₁₄₅).

A compound (L-1) wherein T represents a group represented by T32, R¹ represents a fluorine atom, and R^{3b} represents any substituents indicated in [Table L4] to [Table L12] (hereinafter, referred to as Compound Class SX₂₁₄₆).

A compound (L-1) wherein T represents a group represented by T32, R¹ represents a chlorine atom, and R^{3b} represents any substituents indicated in [Table L4] to [Table L12] (hereinafter, referred to as Compound Class SX₂₁₄₇).

A compound (L-1) wherein T represents a group represented by T32, R¹ represents a bromine atom, and R^{3b} represents any substituents indicated in [Table L4] to [Table L12] (hereinafter, referred to as Compound Class SX₂₁₄₈).

A compound (L-1) wherein T represents a group represented by T32, R¹ represents an iodine atom, and R^{3b} represents any substituents indicated in [Table L4] to [Table L12] (hereinafter, referred to as Compound Class SX₂₁₄₉).

A compound (L-1) wherein T represents a group represented by T33, R¹ represents CF₃, and R³¹ represents any substituents indicated in [Table L4] to [Table L12](hereinafter, referred to as Compound Class SX₂₁₅₀).

A compound (L-1) wherein T represents a group represented by T33, R¹ represents C₂F5, and R³¹ represents any substituents indicated in [Table L4] to [Table L12](hereinafter, referred to as Compound Class SX₂₁₅₁).

A compound (L-1) wherein T represents a group represented by T33, R¹ represents OCF₃, and R^{3b} represents any substituents indicated in [Table L4] to [Table L12](hereinafter, referred to as Compound Class SX₂₁₅₂).

A compound (L-1) wherein T represents a group represented by T33, R¹ represents SCF₃, and R^{3b} represents any substituents indicated in [Table L4] to [Table L12](hereinafter, referred to as Compound Class SX₂₁₅₃).

A compound (L-1) wherein T represents a group represented by T33, R¹ represents S(O)CF₃, and R^{3b} represents any substituents indicated in [Table L4] to [Table L12](hereinafter, referred to as Compound Class SX₂₁₅₃₋₁).

A compound (L-1) wherein T represents a group represented by T33, R¹ represents S(O)₂CF₃, and R^{3b} represents any substituents indicated in [Table L4] to [Table L12](hereinafter, referred to as Compound Class SX₂₁₅₄).

A compound (L-1) wherein T represents a group represented by T33, R¹ represents a fluorine atom, and R^{3b} represents any substituents indicated in [Table L4] to [Table L12] (hereinafter, referred to as Compound Class SX₂₁₅₅).

A compound (L-1) wherein T represents a group represented by T33, R¹ represents a chlorine atom, and R^{3b} represents any substituents indicated in [Table L4] to [Table L12] (hereinafter, referred to as Compound Class SX₂₁₅₆).

A compound (L-1) wherein T represents a group represented by T33, R¹ represents a bromine atom, and R^{3b} represents any substituents indicated in [Table L4] to [Table L12] (hereinafter, referred to as Compound Class SX₂₁₅₇).

A compound (L-1) wherein T represents a group represented by T33, R¹ represents an iodine atom, and R^{3b} represents any substituents indicated in [Table L4] to [Table L12] (hereinafter, referred to as Compound Class SX₂₁₅₈).

A compound (L-1) wherein T represents a group represented by T34, R¹ represents CF₃, and R^{3b} represents any substituents indicated in [Table L4] to [Table L12](hereinafter, referred to as Compound Class SX₂₁₅₉).

A compound (L-1) wherein T represents a group represented by T34, R¹ represents C₂F5, and R^{3b} represents any substituents indicated in [Table L4] to [Table L12](hereinafter, referred to as Compound Class SX₂₁₆₀).

A compound (L-1) wherein T represents a group represented by T34, R¹ represents OCF₃, and R^{3b} represents any substituents indicated in [Table L4] to [Table L12](hereinafter, referred to as Compound Class SX₂₁₆₁).

A compound (L-1) wherein T represents a group represented by T34, R¹ represents SCF₃, and R^{3b} represents any substituents indicated in [Table L4] to [Table L12](hereinafter, referred to as Compound Class SX₂₁₆₂).

A compound (L-1) wherein T represents a group represented by T34, R¹ represents S(O)CF₃, and R^{3b} represents any substituents indicated in [Table L4] to [Table L12](hereinafter, referred to as Compound Class SX₂₁₆₃).

A compound (L-1) wherein T represents a group represented by T34, R¹ represents S(O)₂CF₃, and R^{3b} represents any substituents indicated in [Table L4] to [Table L12](hereinafter, referred to as Compound Class SX₂₁₆₄).

A compound (L-1) wherein T represents a group represented by T34, R¹ represents a fluorine atom, and R^{3b} represents any substituents indicated in [Table L4] to [Table L12] (hereinafter, referred to as Compound Class SX₂₁₆₅).

A compound (L-1) wherein T represents a group represented by T34, R¹ represents a chlorine atom, and R^{3b} represents any substituents indicated in [Table L4] to [Table L12] (hereinafter, referred to as Compound Class SX₂₁₆₆).

A compound (L-1) wherein T represents a group represented by T34, R¹ represents a bromine atom, and R^{3b} represents any substituents indicated in [Table L4] to [Table L12] (hereinafter, referred to as Compound Class SX₂₁₆₇).

A compound (L-1) wherein T represents a group represented by T34, R¹ represents an iodine atom, and R^{3b} represents any substituents indicated in [Table L4] to [Table L12] (hereinafter, referred to as Compound Class SX₂₁₆₈).

A compound (L-1) wherein T represents a group represented by T35, R¹ represents CF₃, and R^{3b} represents any substituents indicated in [Table L4] to [Table L12](hereinafter, referred to as Compound Class SX₂₁₆₉).

A compound (L-1) wherein T represents a group represented by T35, R¹ represents C₂F5, and R^{3b} represents any substituents indicated in [Table L4] to [Table L12](hereinafter, referred to as Compound Class SX₂₁₇₀).

A compound (L-1) wherein T represents a group represented by T35, R¹ represents OCF₃, and R^{3b} represents any substituents indicated in [Table L4] to [Table L12](hereinafter, referred to as Compound Class SX₂₁₇₁).

A compound (L-1) wherein T represents a group represented by T35, R¹ represents SCF₃, and R^{3b} represents any substituents indicated in [Table L4] to [Table L12](hereinafter, referred to as Compound Class SX₂₁₇₂).

A compound (L-1) wherein T represents a group represented by T35, R¹ represents S(O)CF₃, and R^{3b} represents any substituents indicated in [Table L4] to [Table L12](hereinafter, referred to as Compound Class SX₂₁₇₃).

A compound (L-1) wherein T represents a group represented by T35, R¹ represents S(O)₂CF₃, and R^{3b} represents any substituents indicated in [Table L4] to [Table L12](hereinafter, referred to as Compound Class SX₂₁₇₄).

A compound (L-1) wherein T represents a group represented by T35, R¹ represents a fluorine atom, and R^{3b} represents any substituents indicated in [Table L4] to [Table L12] (hereinafter, referred to as Compound Class SX₂₁₇₅).

A compound (L-1) wherein T represents a group represented by T35, R¹ represents a chlorine atom, and R^{3b} represents any substituents indicated in [Table L4] to [Table L12] (hereinafter, referred to as Compound Class SX₂₁₇₆).

A compound (L-1) wherein T represents a group represented by T35, R¹ represents a bromine atom, and R^{3b} represents any substituents indicated in [Table L4] to [Table L12] (hereinafter, referred to as Compound Class SX₂₁₇₇).

A compound (L-1) wherein T represents a group represented by T35, R¹ represents an iodine atom, and R^{3b} represents any substituents indicated in [Table L4] to [Table L12] (hereinafter, referred to as Compound Class SX₂₁₇₈).

A compound (L-1) wherein T represents a group represented by T36, R¹ represents CF₃, and R^{3b} represents any substituents indicated in [Table L4] to [Table L12](hereinafter, referred to as Compound Class SX₂₁₇₉).

A compound (L-1) wherein T represents a group represented by T36, R¹ represents C₂F5, and R^{3b} represents any substituents indicated in [Table L4] to [Table L12](hereinafter, referred to as Compound Class SX₂₁₈₀).

A compound (L-1) wherein T represents a group represented by T36, R¹ represents OCF₃, and R^{3b} represents any substituents indicated in [Table L4] to [Table L12](hereinafter, referred to as Compound Class SX₂₁₈₁).

A compound (L-1) wherein T represents a group represented by T36, R¹ represents SCF₃, and R^{3b} represents any substituents indicated in [Table L4] to [Table L12](hereinafter, referred to as Compound Class SX₂₁₈₂).

A compound (L-1) wherein T represents a group represented by T36, R¹ represents S(O)CF₃, and R^{3b} represents any substituents indicated in [Table L4] to [Table L12](hereinafter, referred to as Compound Class SX₂₁₈₃).

A compound (L-1) wherein T represents a group represented by T36, R¹ represents S(O)₂CF₃, and R^{3b} represents any substituents indicated in [Table L4] to [Table L12](hereinafter, referred to as Compound Class SX₂₁₈₄).

A compound (L-1) wherein T represents a group represented by T36, R¹ represents a fluorine atom, and R^{3b} represents any substituents indicated in [Table L4] to [Table L12] (hereinafter, referred to as Compound Class SX₂₁₈₅).

A compound (L-1) wherein T represents a group represented by T36, R¹ represents a chlorine atom, and R^{3b} represents any substituents indicated in [Table L4] to [Table L12] (hereinafter, referred to as Compound Class SX₂₁₈₆).

A compound (L-1) wherein T represents a group represented by T36, R¹ represents a bromine atom, and R^{3b} represents any substituents indicated in [Table L4] to [Table L12] (hereinafter, referred to as Compound Class SX₂₁₈₇).

A compound (L-1) wherein T represents a group represented by T36, R¹ represents an iodine atom, and R^{3b} represents any substituents indicated in [Table L4] to [Table L12] (hereinafter, referred to as Compound Class SX₂₁₈₈).

A compound (L-2) wherein T represents a group represented by T32, R¹ represents CF₃, and R^{3b} represents any substituents indicated in [Table L4] to [Table L12](hereinafter, referred to as Compound Class SX₂₁₈₉).

A compound (L-2) wherein T represents a group represented by T32, R¹ represents C₂F5, and R^{3b} represents any substituents indicated in [Table L4] to [Table L12](hereinafter, referred to as Compound Class SX₂₁₉₀).

A compound (L-2) wherein T represents a group represented by T32, R¹ represents OCF₃, and R^{3b} represents any substituents indicated in [Table L4] to [Table L12](hereinafter, referred to as Compound Class SX₂₁₉₁).

A compound (L-2) wherein T represents a group represented by T32, R¹ represents SCF₃, and R^{3b} represents any substituents indicated in [Table L4] to [Table L12](hereinafter, referred to as Compound Class SX₂₁₉₂).

A compound (L-2) wherein T represents a group represented by T32, R¹ represents S(O)CF₃, and R^{3b} represents any substituents indicated in [Table L4] to [Table L12](hereinafter, referred to as Compound Class SX₂₁₉₃).

A compound (L-2) wherein T represents a group represented by T32, R¹ represents S(O)₂CF₃, and R^{3b} represents any substituents indicated in [Table L4] to [Table L12](hereinafter, referred to as Compound Class SX₂₁₉₄).

A compound (L-2) wherein T represents a group represented by T32, R¹ represents a fluorine atom, and R^{3b} represents any substituents indicated in [Table L4] to [Table L12] (hereinafter, referred to as Compound Class SX₂₁₉₅).

A compound (L-2) wherein T represents a group represented by T32, R¹ represents a chlorine atom, and R^{3b} represents any substituents indicated in [Table L4] to [Table L12] (hereinafter, referred to as Compound Class SX₂₁₉₆).

A compound (L-2) wherein T represents a group represented by T32, R¹ represents a bromine atom, and R^{3b} represents any substituents indicated in [Table L4] to [Table L12] (hereinafter, referred to as Compound Class SX₂₁₉₇).

A compound (L-2) wherein T represents a group represented by T32, R¹ represents an iodine atom, and R^{3b} represents any substituents indicated in [Table L4] to [Table L12] (hereinafter, referred to as Compound Class SX₂₁₉₈).

A compound (L-2) wherein T represents a group represented by T33, R¹ represents CF₃, and R^{3b} represents any substituents indicated in [Table L4] to [Table L12](hereinafter, referred to as Compound Class SX₂₁₉₉).

A compound (L-2) wherein T represents a group represented by T33, R¹ represents C₂F5, and R^{3b} represents any substituents indicated in [Table L4] to [Table L12](hereinafter, referred to as Compound Class SX₂₂₀₀).

A compound (L-2) wherein T represents a group represented by T33, R¹ represents OCF₃, and R^{3b} represents any substituents indicated in [Table L4] to [Table L12](hereinafter, referred to as Compound Class SX₂₂₀₁).

A compound (L-2) wherein T represents a group represented by T33, R¹ represents SCF₃, and R^{3b} represents any substituents indicated in [Table L4] to [Table L12](hereinafter, referred to as Compound Class SX₂₂₀₂).

A compound (L-2) wherein T represents a group represented by T33, R¹ represents S(O)CF₃, and R^{3b} represents any substituents indicated in [Table L4] to [Table L12](hereinafter, referred to as Compound Class SX₂₂₀₃).

A compound (L-2) wherein T represents a group represented by T33, R¹ represents S(O)₂CF₃, and R^{3b} represents any substituents indicated in [Table L4] to [Table L12](hereinafter, referred to as Compound Class SX₂₂₀₄).

A compound (L-2) wherein T represents a group represented by T33, R¹ represents a fluorine atom, and R^{3b} represents any substituents indicated in [Table L4] to [Table L12] (hereinafter, referred to as Compound Class SX₂₂₀₅).

A compound (L-2) wherein T represents a group represented by T33, R¹ represents a chlorine atom, and R^{3b} represents any substituents indicated in [Table L4] to [Table L12] (hereinafter, referred to as Compound Class SX₂₂₀₆).

A compound (L-2) wherein T represents a group represented by T33, R¹ represents a bromine atom, and R^{3b} represents any substituents indicated in [Table L4] to [Table L12] (hereinafter, referred to as Compound Class SX₂₂₀₇).

A compound (L-2) wherein T represents a group represented by T33, R¹ represents an iodine atom, and R^{3b} represents any substituents indicated in [Table L4] to [Table L12] (hereinafter, referred to as Compound Class SX₂₂₀₈).

A compound (L-2) wherein T represents a group represented by T34, R¹ represents CF₃, and R^{3b} represents any substituents indicated in [Table L4] to [Table L12](hereinafter, referred to as Compound Class SX₂₂₀₉).

A compound (L-2) wherein T represents a group represented by T34, R¹ represents C₂F5, and R^{3b} represents any substituents indicated in [Table L4] to [Table L12](hereinafter, referred to as Compound Class SX₂₂₁₀).

A compound (L-2) wherein T represents a group represented by T34, R¹ represents OCF₃, and R^{3b} represents any substituents indicated in [Table L4] to [Table L12](hereinafter, referred to as Compound Class SX₂₂₁₁).

A compound (L-2) wherein T represents a group represented by T34, R¹ represents SCF₃, and R^{3b} represents any substituents indicated in [Table L4] to [Table L12](hereinafter, referred to as Compound Class SX₂₂₁₂).

A compound (L-2) wherein T represents a group represented by T34, R¹ represents S(O)CF₃, and R^{3b} represents any substituents indicated in [Table L4] to [Table L12](hereinafter, referred to as Compound Class SX₂₂₁₃).

A compound (L-2) wherein T represents a group represented by T34, R¹ represents S(O)₂CF₃, and R^{3b} represents any substituents indicated in [Table L4] to [Table L12](hereinafter, referred to as Compound Class SX₂₂₁₄).

A compound (L-2) wherein T represents a group represented by T34, R¹ represents a fluorine atom, and R^{3b} represents any substituents indicated in [Table L4] to [Table L12] (hereinafter, referred to as Compound Class SX₂₂₁₅).

A compound (L-2) wherein T represents a group represented by T34, R¹ represents a chlorine atom, and R^{3b} represents any substituents indicated in [Table L4] to [Table L12] (hereinafter, referred to as Compound Class SX₂₂₁₆).

A compound (L-2) wherein T represents a group represented by T34, R¹ represents a bromine atom, and R^{3b} represents any substituents indicated in [Table L4] to [Table L12] (hereinafter, referred to as Compound Class SX₂₂₁₇).

A compound (L-2) wherein T represents a group represented by T34, R¹ represents an iodine atom, and R^{3b} represents any substituents indicated in [Table L4] to [Table L12] (hereinafter, referred to as Compound Class SX₂₂₁₈).

A compound (L-2) wherein T represents a group represented by T35, R¹ represents CF₃, and R^{3b} represents any substituents indicated in [Table L4] to [Table L12](hereinafter, referred to as Compound Class SX₂₂₁₉).

A compound (L-2) wherein T represents a group represented by T35, R¹ represents C₂F5, and R^{3b} represents any substituents indicated in [Table L4] to [Table L12](hereinafter, referred to as Compound Class SX₂₂₂₀).

A compound (L-2) wherein T represents a group represented by T35, R¹ represents OCF₃, and R^{3b} represents any substituents indicated in [Table L4] to [Table L12](hereinafter, referred to as Compound Class SX₂₂₂₁).

A compound (L-2) wherein T represents a group represented by T35, R¹ represents SCF₃, and R^{3b} represents any substituents indicated in [Table L4] to [Table L12](hereinafter, referred to as Compound Class SX₂₂₂₂).

A compound (L-2) wherein T represents a group represented by T35, R¹ represents S(O)CF₃, and R^{3b} represents any substituents indicated in [Table L4] to [Table L12](hereinafter, referred to as Compound Class SX₂₂₂₃).

A compound (L-2) wherein T represents a group represented by T35, R¹ represents S(O)₂CF₃, and R^{3b} represents any substituents indicated in [Table L4] to [Table L12](hereinafter, referred to as Compound Class SX₂₂₂₄).

A compound (L-2) wherein T represents a group represented by T35, R¹ represents a fluorine atom, and R^{3b} represents any substituents indicated in [Table L4] to [Table L12] (hereinafter, referred to as Compound Class SX₂₂₂₅).

A compound (L-2) wherein T represents a group represented by T35, R¹ represents a chlorine atom, and R^{3b} represents any substituents indicated in [Table L4] to [Table L12] (hereinafter, referred to as Compound Class SX₂₂₂₆).

A compound (L-2) wherein T represents a group represented by T35, R¹ represents a bromine atom, and R^{3b} represents any substituents indicated in [Table L4] to [Table L12] (hereinafter, referred to as Compound Class SX₂₂₂₇).

A compound (L-2) wherein T represents a group represented by T36, R¹ represents an iodine atom, and R^{3b} represents any substituents indicated in [Table L4] to [Table L12] (hereinafter, referred to as Compound Class SX₂₂₂₈).

A compound (L-2) wherein T represents a group represented by T36, R¹ represents CF₃, and R^{3b} represents any substituents indicated in [Table L4] to [Table L12](hereinafter, referred to as Compound Class SX₂₂₂₉).

A compound (L-2) wherein T represents a group represented by T36, R¹ represents C₂F5, and R^{3b} represents any substituents indicated in [Table L4] to [Table L12](hereinafter, referred to as Compound Class SX₂₂₃₀).

A compound (L-2) wherein T represents a group represented by T36, R¹ represents OCF₃, and R^{3b} represents any substituents indicated in [Table L4] to [Table L12](hereinafter, referred to as Compound Class SX₂₂₃₁).

A compound (L-2) wherein T represents a group represented by T36, R¹ represents SCF₃, and R^{3b} represents any substituents indicated in [Table L4] to [Table L12](hereinafter, referred to as Compound Class SX₂₂₃₂).

A compound (L-2) wherein T represents a group represented by T36, R¹ represents S(O)CF₃, and R^{3b} represents any substituents indicated in [Table L4] to [Table L12](hereinafter, referred to as Compound Class SX₂₂₃₃).

A compound (L-2) wherein T represents a group represented by T36, R¹ represents S(O)₂CF₃, and R^{3b} represents any substituents indicated in [Table L4] to [Table L12](hereinafter, referred to as Compound Class SX₂₂₃₄).

A compound (L-2) wherein T represents a group represented by T36, R¹ represents a fluorine atom, and R^{3b} represents any substituents indicated in [Table L4] to [Table L12] (hereinafter, referred to as Compound Class SX₂₂₃₅).

A compound (L-2) wherein T represents a group represented by T36, R¹ represents a chlorine atom, and R^{3b} represents any substituents indicated in [Table L4] to [Table L12] (hereinafter, referred to as Compound Class SX₂₂₃₆).

A compound (L-2) wherein T represents a group represented by T36, R¹ represents a bromine atom, and R^{3b} represents any substituents indicated in [Table L4] to [Table L12] (hereinafter, referred to as Compound Class SX₂₂₃₇).

A compound (L-2) wherein T represents a group represented by T36, R¹ represents an iodine atom, and R^{3b} represents any substituents indicated in [Table L4] to [Table L12] (hereinafter, referred to as Compound Class SX₂₂₃₈).

A compound (L-3) wherein T represents a group represented by T32, R¹ represents CF₃, and R^{3b} represents any substituents indicated in [Table L4] to [Table L12](hereinafter, referred to as Compound Class SX₂₂₃₉).

A compound (L-3) wherein T represents a group represented by T32, R¹ represents C₂F5, and R^{3b} represents any substituents indicated in [Table L4] to [Table L12](hereinafter, referred to as Compound Class SX₂₂₄₀).

A compound (L-3) wherein T represents a group represented by T32, R¹ represents OCF₃, and R^{3b} represents any substituents indicated in [Table L4] to [Table L12](hereinafter, referred to as Compound Class SX₂₂₄₁).

A compound (L-3) wherein T represents a group represented by T32, R¹ represents SCF₃, and R^{3b} represents any substituents indicated in [Table L4] to [Table L12](hereinafter, referred to as Compound Class SX₂₂₄₂).

A compound (L-3) wherein T represents a group represented by T32, R¹ represents S(O)CF₃, and R^{3b} represents any substituents indicated in [Table L4] to [Table L12](hereinafter, referred to as Compound Class SX₂₂₄₃).

A compound (L-3) wherein T represents a group represented by T32, R¹ represents S(O)₂CF₃, and R^{3b} represents any substituents indicated in [Table L4] to [Table L12](hereinafter, referred to as Compound Class SX₂₂₄₄).

A compound (L-3) wherein T represents a group represented by T32, R¹ represents a fluorine atom, and R^{3b} represents any substituents indicated in [Table L4] to [Table L12] (hereinafter, referred to as Compound Class SX₂₂₄₅).

A compound (L-3) wherein T represents a group represented by T32, R¹ represents a chlorine atom, and R^{3b} represents any substituents indicated in [Table L4] to [Table L12] (hereinafter, referred to as Compound Class SX₂₂₄₆).

A compound (L-3) wherein T represents a group represented by T32, R¹ represents a bromine atom, and R^{3b} represents any substituents indicated in [Table L4] to [Table L12] (hereinafter, referred to as Compound Class SX₂₂₄₇).

A compound (L-3) wherein T represents a group represented by T32, R¹ represents an iodine atom, and R^{3b} represents any substituents indicated in [Table L4] to [Table L12] (hereinafter, referred to as Compound Class SX₂₂₄₈).

A compound (L-3) wherein T represents a group represented by T33, R¹ represents CF₃, and R^{3b} represents any substituents indicated in [Table L4] to [Table L12](hereinafter, referred to as Compound Class SX₂₂₄₉).

A compound (L-3) wherein T represents a group represented by T33, R¹ represents C₂F5, and R^{3b} represents any substituents indicated in [Table L4] to [Table L12](hereinafter, referred to as Compound Class SX₂₂₅₀).

A compound (L-3) wherein T represents a group represented by T33, R¹ represents OCF₃, and R^{3b} represents any substituents indicated in [Table L4] to [Table L12](hereinafter, referred to as Compound Class SX₂₂₅₁).

A compound (L-3) wherein T represents a group represented by T33, R¹ represents SCF₃, and R^{3b} represents any substituents indicated in [Table L4] to [Table L12](hereinafter, referred to as Compound Class SX₂₂₅₂).

A compound (L-3) wherein T represents a group represented by T33, R¹ represents S(O)CF₃, and R^{3b} represents any substituents indicated in [Table L4] to [Table L12](hereinafter, referred to as Compound Class SX₂₂₅₃).

A compound (L-3) wherein T represents a group represented by T33, R¹ represents S(O)₂CF₃, and R^{3b} represents any substituents indicated in [Table L4] to [Table L12](hereinafter, referred to as Compound Class SX₂₂₅₄).

A compound (L-3) wherein T represents a group represented by T33, R¹ represents a fluorine atom, and R^{3b} represents any substituents indicated in [Table L4] to [Table L12] (hereinafter, referred to as Compound Class SX₂₂₅₅).

A compound (L-3) wherein T represents a group represented by T33, R¹ represents a chlorine atom, and R^{3b} represents any substituents indicated in [Table L4] to [Table L12] (hereinafter, referred to as Compound Class SX₂₂₅₆).

A compound (L-3) wherein T represents a group represented by T33, R¹ represents a bromine atom, and R^{3b} represents any substituents indicated in [Table L4] to [Table L12] (hereinafter, referred to as Compound Class SX₂₂₅₇).

A compound (L-3) wherein T represents a group represented by T33, R¹ represents an iodine atom, and R^{3b} represents any substituents indicated in [Table L4] to [Table L12] (hereinafter, referred to as Compound Class SX₂₂₅₈).

A compound (L-3) wherein T represents a group represented by T34, R¹ represents CF₃, and R^{3b} represents any substituents indicated in [Table L4] to [Table L12](hereinafter, referred to as Compound Class SX₂₂₅₉).

A compound (L-3) wherein T represents a group represented by T35, R¹ represents C₂F5, and R^{3b} represents any substituents indicated in [Table L4] to [Table L12](hereinafter, referred to as Compound Class SX₂₂₆₀).

A compound (L-3) wherein T represents a group represented by T34, R¹ represents OCF₃, and R^{3b} represents any substituents indicated in [Table L4] to [Table L12](hereinafter, referred to as Compound Class SX₂₂₆₁).

A compound (L-3) wherein T represents a group represented by T34, R¹ represents SCF₃, and R^{3b} represents any substituents indicated in [Table L4] to [Table L12](hereinafter, referred to as Compound Class SX₂₂₆₂).

A compound (L-3) wherein T represents a group represented by T34, R¹ represents S(O)CF₃, and R^{3b} represents any substituents indicated in [Table L4] to [Table L12](hereinafter, referred to as Compound Class SX₂₂₆₃).

A compound (L-3) wherein T represents a group represented by T34, R¹ represents S(O)₂CF₃, and R^{3b} represents any substituents indicated in [Table L4] to [Table L12](hereinafter, referred to as Compound Class SX₂₂₆₄).

A compound (L-3) wherein T represents a group represented by T34, R¹ represents a fluorine atom, and R^{3b} represents any substituents indicated in [Table L4] to [Table L12] (hereinafter, referred to as Compound Class SX₂₂₆₅).

A compound (L-3) wherein T represents a group represented by T34, R¹ represents a chlorine atom, and R^{3b} represents any substituents indicated in [Table L4] to [Table L12] (hereinafter, referred to as Compound Class SX₂₂₆₆).

A compound (L-3) wherein T represents a group represented by T34, R¹ represents a bromine atom, and R^{3b} represents any substituents indicated in [Table L4] to [Table L12] (hereinafter, referred to as Compound Class SX₂₂₆₇).

A compound (L-3) wherein T represents a group represented by T34, R¹ represents an iodine atom, and R^{3b} represents any substituents indicated in [Table L4] to [Table L12] (hereinafter, referred to as Compound Class SX₂₂₆₈).

A compound (L-3) wherein T represents a group represented by T35, R¹ represents CF₃, and R^{3b} represents any substituents indicated in [Table L4] to [Table L12](hereinafter, referred to as Compound Class SX₂₂₆₉).

A compound (L-3) wherein T represents a group represented by T35, R¹ represents C₂F5, and R^{3b} represents any substituents indicated in [Table L4] to [Table L12](hereinafter, referred to as Compound Class SX₂₂₇₀).

A compound (L-3) wherein T represents a group represented by T35, R¹ represents OCF₃, and R^{3b} represents any substituents indicated in [Table L4] to [Table L12](hereinafter, referred to as Compound Class SX₂₂₇₁).

A compound (L-3) wherein T represents a group represented by T35, R¹ represents SCF₃, and R^{3b} represents any substituents indicated in [Table L4] to [Table L12](hereinafter, referred to as Compound Class SX₂₂₇₂).

A compound (L-3) wherein T represents a group represented by T35, R¹ represents S(O)CF₃, and R^{3b} represents any substituents indicated in [Table L4] to [Table L12](hereinafter, referred to as Compound Class SX₂₂₇₃).

A compound (L-3) wherein T represents a group represented by T35, R¹ represents S(O)₂CF₃, and R^{3b} represents any substituents indicated in [Table L4] to [Table L12](hereinafter, referred to as Compound Class SX₂₂₇₄).

A compound (L-3) wherein T represents a group represented by T35, R¹ represents a fluorine atom, and R^{3b} represents any substituents indicated in [Table L4] to [Table L12] (hereinafter, referred to as Compound Class SX₂₂₇₅).

A compound (L-3) wherein T represents a group represented by T35, R¹ represents a chlorine atom, and R^{3b} represents any substituents indicated in [Table L4] to [Table L12] (hereinafter, referred to as Compound Class SX₂₂₇₆).

A compound (L-3) wherein T represents a group represented by T35, R¹ represents a bromine atom, and R^{3b} represents any substituents indicated in [Table L4] to [Table L12] (hereinafter, referred to as Compound Class SX₂₂₇₇).

A compound (L-3) wherein T represents a group represented by T35, R¹ represents an iodine atom, and R^{3b} represents any substituents indicated in [Table L4] to [Table L12] (hereinafter, referred to as Compound Class SX₂₂₇₈).

A compound (L-3) wherein T represents a group represented by T36, R¹ represents CF₃, and R^{3b} represents any substituents indicated in [Table L4] to [Table L12](hereinafter, referred to as Compound Class SX₂₂₇₉).

A compound (L-3) wherein T represents a group represented by T36, R¹ represents C₂F5, and R^{3b} represents any substituents indicated in [Table L4] to [Table L12](hereinafter, referred to as Compound Class SX₂₂₈₀).

A compound (L-3) wherein T represents a group represented by T36, R¹ represents OCF₃, and R^{3b} represents any substituents indicated in [Table L4] to [Table L12](hereinafter, referred to as Compound Class SX₂₂₈₁).

A compound (L-3) wherein T represents a group represented by T36, R¹ represents SCF₃, and R^{3b} represents any substituents indicated in [Table L4] to [Table L12](hereinafter, referred to as Compound Class SX₂₂₈₂).

A compound (L-3) wherein T represents a group represented by T36, R¹ represents S(O)CF₃, and R^{3b} represents any substituents indicated in [Table L4] to [Table L12](hereinafter, referred to as Compound Class SX₂₂₈₃).

A compound (L-3) wherein T represents a group represented by T36, R¹ represents S(O)₂CF₃, and R^{3b} represents any substituents indicated in [Table L4] to [Table L12](hereinafter, referred to as Compound Class SX₂₂₈₄).

A compound (L-3) wherein T represents a group represented by T36, R¹ represents a fluorine atom, and R^{3b} represents any substituents indicated in [Table L4] to [Table L12] (hereinafter, referred to as Compound Class SX₂₂₈₅).

A compound (L-3) wherein T represents a group represented by T36, R¹ represents a chlorine atom, and R^{3b} represents any substituents indicated in [Table L4] to [Table L12] (hereinafter, referred to as Compound Class SX₂₂₈₆).

A compound (L-3) wherein T represents a group represented by T36, R¹ represents a bromine atom, and R^{3b} represents any substituents indicated in [Table L4] to [Table L12] (hereinafter, referred to as Compound Class SX₂₂₈₇).

A compound (L-3) wherein T represents a group represented by T36, R¹ represents an iodine atom, and R^{3b} represents any substituents indicated in [Table L4] to [Table L12] (hereinafter, referred to as Compound Class SX₂₂₈₈).

A compound (L-4) wherein T represents a group represented by T32, R¹ represents CF₃, and R^{3b} represents any substituents indicated in [Table L4] to [Table L12](hereinafter, referred to as Compound Class SX₂₂₈₉).

A compound (L-4) wherein T represents a group represented by T32, R¹ represents C₂F5, and R^{3b} represents any substituents indicated in [Table L4] to [Table L12](hereinafter, referred to as Compound Class SX₂₂₉₀).

A compound (L-4) wherein T represents a group represented by T32, R¹ represents OCF₃, and R^{3b} represents any substituents indicated in [Table L4] to [Table L12](hereinafter, referred to as Compound Class SX₂₂₉₁).

A compound (L-4) wherein T represents a group represented by T32, R¹ represents SCF₃, and R^{3b} represents any substituents indicated in [Table L4] to [Table L12](hereinafter, referred to as Compound Class SX₂₂₉₂).

A compound (L-4) wherein T represents a group represented by T32, R¹ represents S(O)CF₃, and R^{3b} represents any substituents indicated in [Table L4] to [Table L12](hereinafter, referred to as Compound Class SX₂₂₉₃).

A compound (L-4) wherein T represents a group represented by T32, R¹ represents S(O)₂CF₃, and R^{3b} represents any substituents indicated in [Table L4] to [Table L12](hereinafter, referred to as Compound Class SX₂₂₉₄).

A compound (L-4) wherein T represents a group represented by T32, R¹ represents a fluorine atom, and R^{3b} represents any substituents indicated in [Table L4] to [Table L12] (hereinafter, referred to as Compound Class SX₂₂₉₅).

A compound (L-4) wherein T represents a group represented by T32, R¹ represents a chlorine atom, and R^{3b} represents any substituents indicated in [Table L4] to [Table L12] (hereinafter, referred to as Compound Class SX₂₂₉₆).

A compound (L-4) wherein T represents a group represented by T32, R¹ represents a bromine atom, and R^{3b} represents any substituents indicated in [Table L4] to [Table L12] (hereinafter, referred to as Compound Class SX₂₂₉₇).

A compound (L-4) wherein T represents a group represented by T32, R¹ represents an iodine atom, and R^{3b} represents any substituents indicated in [Table L4] to [Table L12] (hereinafter, referred to as Compound Class SX₂₂₉₈).

A compound (L-4) wherein T represents a group represented by T33, R¹ represents CF₃, and R^{3b} represents any substituents indicated in [Table L4] to [Table L12](hereinafter, referred to as Compound Class SX₂₂₉₉).

A compound (L-4) wherein T represents a group represented by T33, R¹ represents C₂F5, and R^{3b} represents any substituents indicated in [Table L4] to [Table L12](hereinafter, referred to as Compound Class SX₂₃₀₀).

A compound (L-4) wherein T represents a group represented by T33, R¹ represents OCF₃, and R^{3b} represents any substituents indicated in [Table L4] to [Table L12](hereinafter, referred to as Compound Class SX₂₃₀₁).

A compound (L-4) wherein T represents a group represented by T33, R¹ represents SCF₃, and R^{3b} represents any substituents indicated in [Table L4] to [Table L12](hereinafter, referred to as Compound Class SX₂₃₀₂).

A compound (L-4) wherein T represents a group represented by T33, R¹ represents S(O)CF₃, and R^{3b} represents any substituents indicated in [Table L4] to [Table L12](hereinafter, referred to as Compound Class SX₂₃₀₃).

A compound (L-4) wherein T represents a group represented by T33, R¹ represents S(O)₂CF₃, and R^{3b} represents any substituents indicated in [Table L4] to [Table L12](hereinafter, referred to as Compound Class SX₂₃₀₄).

A compound (L-4) wherein T represents a group represented by T33, R¹ represents a fluorine atom, and R^{3b} represents any substituents indicated in [Table L4] to [Table L12] (hereinafter, referred to as Compound Class SX₂₃₀₅).

A compound (L-4) wherein T represents a group represented by T33, R¹ represents a chlorine atom, and R^{3b} represents any substituents indicated in [Table L4] to [Table L12] (hereinafter, referred to as Compound Class SX₂₃₀₆).

A compound (L-4) wherein T represents a group represented by T33, R¹ represents a bromine atom, and R^{3b} represents any substituents indicated in [Table L4] to [Table L12] (hereinafter, referred to as Compound Class SX₂₃₀₇).

A compound (L-4) wherein T represents a group represented by T33, R¹ represents an iodine atom, and R^{3b} represents any substituents indicated in [Table L4] to [Table L12] (hereinafter, referred to as Compound Class SX₂₃₀₈).

A compound (L-4) wherein T represents a group represented by T34, R¹ represents CF₃, and R^{3b} represents any substituents indicated in [Table L4] to [Table L12](hereinafter, referred to as Compound Class SX₂₃₀₉).

A compound (L-4) wherein T represents a group represented by T34, R¹ represents C₂F5, and R^{3b} represents any substituents indicated in [Table L4] to [Table L12](hereinafter, referred to as Compound Class SX₂₃₁₀).

A compound (L-4) wherein T represents a group represented by T34, R¹ represents OCF₃, and R^{3b} represents any substituents indicated in [Table L4] to [Table L12](hereinafter, referred to as Compound Class SX₂₃₁₁).

A compound (L-4) wherein T represents a group represented by T34, R¹ represents SCF₃, and R^{3b} represents any substituents indicated in [Table L4] to [Table L12](hereinafter, referred to as Compound Class SX₂₃₁₂).

A compound (L-4) wherein T represents a group represented by T34, R¹ represents S(O)CF₃, and R^{3b} represents any substituents indicated in [Table L4] to [Table L12](hereinafter, referred to as Compound Class SX₂₃₁₃).

A compound (L-4) wherein T represents a group represented by T34, R¹ represents S(O)₂CF₃, and R^{3b} represents any substituents indicated in [Table L4] to [Table L12](hereinafter, referred to as Compound Class SX₂₃₁₄).

A compound (L-4) wherein T represents a group represented by T34, R¹ represents a fluorine atom, and R^{3b} represents any substituents indicated in [Table L4] to [Table L12] (hereinafter, referred to as Compound Class SX₂₃₁₅).

A compound (L-4) wherein T represents a group represented by T34, R¹ represents a chlorine atom, and R^{3b} represents any substituents indicated in [Table L4] to [Table L12] (hereinafter, referred to as Compound Class SX₂₃₁₆).

A compound (L-4) wherein T represents a group represented by T34, R¹ represents a bromine atom, and R^{3b} represents any substituents indicated in [Table L4] to [Table L12] (hereinafter, referred to as Compound Class SX₂₃₁₇).

A compound (L-4) wherein T represents a group represented by T34, R¹ represents an iodine atom, and R^{3b} represents any substituents indicated in [Table L4] to [Table L12] (hereinafter, referred to as Compound Class SX₂₃₁₈).

A compound (L-4) wherein T represents a group represented by T35, R¹ represents CF₃, and R^{3b} represents any substituents indicated in [Table L4] to [Table L12](hereinafter, referred to as Compound Class SX₂₃₁₉).

A compound (L-4) wherein T represents a group represented by T35, R¹ represents C₂F5, and R^{3b} represents any substituents indicated in [Table L4] to [Table L12](hereinafter, referred to as Compound Class SX₂₃₂₀).

A compound (L-4) wherein T represents a group represented by T35, R¹ represents OCF₃, and R^{3b} represents any substituents indicated in [Table L4] to [Table L12](hereinafter, referred to as Compound Class SX₂₃₂₁).

A compound (L-4) wherein T represents a group represented by T35, R¹ represents SCF₃, and R^{3b} represents any substituents indicated in [Table L4] to [Table L12](hereinafter, referred to as Compound Class SX₂₃₂₂).

A compound (L-4) wherein T represents a group represented by T35, R¹ represents S(O)CF₃, and R^{3b} represents any substituents indicated in [Table L4] to [Table L12](hereinafter, referred to as Compound Class SX₂₃₂₃).

A compound (L-4) wherein T represents a group represented by T35, R¹ represents S(O)₂CF₃, and R^{3b} represents any substituents indicated in [Table L4] to [Table L12](hereinafter, referred to as Compound Class SX₂₃₂₄).

A compound (L-4) wherein T represents a group represented by T35, R¹ represents a fluorine atom, and R^{3b} represents any substituents indicated in [Table L4] to [Table L12] (hereinafter, referred to as Compound Class SX₂₃₂₅).

A compound (L-4) wherein T represents a group represented by T35, R¹ represents a chlorine atom, and R^{3b} represents any substituents indicated in [Table L4] to [Table L12] (hereinafter, referred to as Compound Class SX₂₃₂₆).

A compound (L-4) wherein T represents a group represented by T35, R¹ represents a bromine atom, and R^{3b} represents any substituents indicated in [Table L4] to [Table L12] (hereinafter, referred to as Compound Class SX₂₃₂₇).

A compound (L-4) wherein T represents a group represented by T35, R¹ represents an iodine atom, and R^{3b} represents any substituents indicated in [Table L4] to [Table L12] (hereinafter, referred to as Compound Class SX₂₃₂₈).

A compound (L-4) wherein T represents a group represented by T36, R¹ represents CF₃, and R^{3b} represents any substituents indicated in [Table L4] to [Table L12](hereinafter, referred to as Compound Class SX₂₃₂₉).

A compound (L-4) wherein T represents a group represented by T36, R¹ represents C₂F5, and R^{3b} represents any substituents indicated in [Table L4] to [Table L12](hereinafter, referred to as Compound Class SX₂₃₃₀).

A compound (L-4) wherein T represents a group represented by T36, R¹ represents OCF₃, and R^{3b} represents any substituents indicated in [Table L4] to [Table L12](hereinafter, referred to as Compound Class SX₂₃₃₁).

A compound (L-4) wherein T represents a group represented by T36, R¹ represents SCF₃, and R^{3b} represents any substituents indicated in [Table L4] to [Table L12](hereinafter, referred to as Compound Class SX₂₃₃₂).

A compound (L-4) wherein T represents a group represented by T36, R¹ represents S(O)CF₃, and R^{3b} represents any substituents indicated in [Table L4] to [Table L12](hereinafter, referred to as Compound Class SX₂₃₃₃).

A compound (L-4) wherein T represents a group represented by T36, R¹ represents S(O)₂CF₃, and R^{3b} represents any substituents indicated in [Table L4] to [Table L12](hereinafter, referred to as Compound Class SX₂₃₃₄).

A compound (L-4) wherein T represents a group represented by T36, R¹ represents a fluorine atom, and R^{3b} represents any substituents indicated in [Table L4] to [Table L12] (hereinafter, referred to as Compound Class SX₂₃₃₅).

A compound (L-4) wherein T represents a group represented by T36, R¹ represents a chlorine atom, and R^{3b} represents any substituents indicated in [Table L4] to [Table L12] (hereinafter, referred to as Compound Class SX₂₃₃₆).

A compound (L-4) wherein T represents a group represented by T36, R¹ represents a bromine atom, and R^{3b} represents any substituents indicated in [Table L4] to [Table L12] (hereinafter, referred to as Compound Class SX₂₃₃₇).

A compound (L-4) wherein T represents a group represented by T36, R¹ represents an iodine atom, and R^{3b} represents any substituents indicated in [Table L4] to [Table L12] (hereinafter, referred to as Compound Class SX₂₃₃₈).

A compound (L-5) wherein T represents a group represented by T32, R¹ represents CF₃, and R^{3b} represents any substituents indicated in [Table L4] to [Table L12](hereinafter, referred to as Compound Class SX₂₃₃₉).

A compound (L-5) wherein T represents a group represented by T32, R¹ represents C₂F5, and R^{3b} represents any substituents indicated in [Table L4] to [Table L12](hereinafter, referred to as Compound Class SX₂₃₄₀).

A compound (L-5) wherein T represents a group represented by T32, R¹ represents OCF₃, and R^{3b} represents any substituents indicated in [Table L4] to [Table L12](hereinafter, referred to as Compound Class SX₂₃₄₁).

A compound (L-5) wherein T represents a group represented by T32, R¹ represents SCF₃, and R^{3b} represents any substituents indicated in [Table L4] to [Table L12](hereinafter, referred to as Compound Class SX₂₃₄₂).

A compound (L-5) wherein T represents a group represented by T32, R¹ represents S(O)CF₃, and R^{3b} represents any substituents indicated in [Table L4] to [Table L12](hereinafter, referred to as Compound Class SX₂₃₄₃).

A compound (L-5) wherein T represents a group represented by T32, R¹ represents S(O)₂CF₃, and R^{3b} represents any substituents indicated in [Table L4] to [Table L12](hereinafter, referred to as Compound Class SX₂₃₄₄).

A compound (L-5) wherein T represents a group represented by T32, R¹ represents a fluorine atom, and R^{3b} represents any substituents indicated in [Table L4] to [Table L12] (hereinafter, referred to as Compound Class SX₂₃₄₅).

A compound (L-5) wherein T represents a group represented by T32, R¹ represents a chlorine atom, and R^{3b} represents any substituents indicated in [Table L4] to [Table L12] (hereinafter, referred to as Compound Class SX₂₃₄₆).

A compound (L-5) wherein T represents a group represented by T32, R¹ represents a bromine atom, and R^{3b} represents any substituents indicated in [Table L4] to [Table L12] (hereinafter, referred to as Compound Class SX₂₃₄₇).

A compound (L-5) wherein T represents a group represented by T32, R¹ represents an iodine atom, and R^{3b} represents any substituents indicated in [Table L4] to [Table L12] (hereinafter, referred to as Compound Class SX₂₃₄₈).

A compound (L-5) wherein T represents a group represented by T33, R¹ represents CF₃, and R^{3b} represents any substituents indicated in [Table L4] to [Table L12](hereinafter, referred to as Compound Class SX₂₃₄₉).

A compound (L-5) wherein T represents a group represented by T33, R¹ represents C₂F5, and R^{3b} represents any substituents indicated in [Table L4] to [Table L12](hereinafter, referred to as Compound Class SX₂₃₅₀).

A compound (L-5) wherein T represents a group represented by T33, R¹ represents OCF₃, and R^{3b} represents any substituents indicated in [Table L4] to [Table L12](hereinafter, referred to as Compound Class SX₂₃₅₁).

A compound (L-5) wherein T represents a group represented by T33, R¹ represents SCF₃, and R^{3b} represents any substituents indicated in [Table L4] to [Table L12](hereinafter, referred to as Compound Class SX₂₃₅₂).

A compound (L-5) wherein T represents a group represented by T33, R¹ represents S(O)CF₃, and R^{3b} represents any substituents indicated in [Table L4] to [Table L12](hereinafter, referred to as Compound Class SX₂₃₅₃).

A compound (L-5) wherein T represents a group represented by T33, R¹ represents S(O)₂CF₃, and R^{3b} represents any substituents indicated in [Table L4] to [Table L12](hereinafter, referred to as Compound Class SX₂₃₅₄).

A compound (L-5) wherein T represents a group represented by T33, R¹ represents a fluorine atom, and R^{3b} represents any substituents indicated in [Table L4] to [Table L12] (hereinafter, referred to as Compound Class SX₂₃₅₅).

A compound (L-5) wherein T represents a group represented by T33, R¹ represents a chlorine atom, and R^{3b} represents any substituents indicated in [Table L4] to [Table L12] (hereinafter, referred to as Compound Class SX₂₃₅₆).

A compound (L-5) wherein T represents a group represented by T33, R¹ represents a bromine atom, and R^{3b} represents any substituents indicated in [Table L4] to [Table L12] (hereinafter, referred to as Compound Class SX₂₃₅₇).

A compound (L-5) wherein T represents a group represented by T33, R¹ represents an iodine atom, and R^{3b} represents any substituents indicated in [Table L4] to [Table L12] (hereinafter, referred to as Compound Class SX₂₃₅₈).

A compound (L-5) wherein T represents a group represented by T34, R¹ represents CF₃, and R^{3b} represents any substituents indicated in [Table L4] to [Table L12](hereinafter, referred to as Compound Class SX₂₃₅₉).

A compound (L-5) wherein T represents a group represented by T34, R¹ represents C₂F5, and R^{3b} represents any substituents indicated in [Table L4] to [Table L12](hereinafter, referred to as Compound Class SX₂₃₆₀).

A compound (L-5) wherein T represents a group represented by T34, R¹ represents OCF₃, and R^{3b} represents any substituents indicated in [Table L4] to [Table L12](hereinafter, referred to as Compound Class SX₂₃₆₁).

A compound (L-5) wherein T represents a group represented by T34, R¹ represents SCF₃, and R^{3b} represents any substituents indicated in [Table L4] to [Table L12](hereinafter, referred to as Compound Class SX₂₃₆₂).

A compound (L-5) wherein T represents a group represented by T34, R¹ represents S(O)CF₃, and R^{3b} represents any substituents indicated in [Table L4] to [Table L12](hereinafter, referred to as Compound Class SX₂₃₆₃).

A compound (L-5) wherein T represents a group represented by T34, R¹ represents S(O)₂CF₃, and R^{3b} represents any substituents indicated in [Table L4] to [Table L12](hereinafter, referred to as Compound Class SX₂₃₆₄).

A compound (L-5) wherein T represents a group represented by T34, R¹ represents a fluorine atom, and R^{3b} represents any substituents indicated in [Table L4] to [Table L12] (hereinafter, referred to as Compound Class SX₂₃₆₅).

A compound (L-5) wherein T represents a group represented by T34, R¹ represents a chlorine atom, and R^{3b} represents any substituents indicated in [Table L4] to [Table L12] (hereinafter, referred to as Compound Class SX₂₃₆₆).

A compound (L-5) wherein T represents a group represented by T34, R¹ represents a bromine atom, and R^{3b} represents any substituents indicated in [Table L4] to [Table L12] (hereinafter, referred to as Compound Class SX₂₃₆₇).

A compound (L-5) wherein T represents a group represented by T34, R¹ represents an iodine atom, and R^{3b} represents any substituents indicated in [Table L4] to [Table L12] (hereinafter, referred to as Compound Class SX₂₃₆₈).

A compound (L-5) wherein T represents a group represented by T35, R¹ represents CF₃, and R^{3b} represents any substituents indicated in [Table L4] to [Table L12](hereinafter, referred to as Compound Class SX₂₃₆₉).

A compound (L-5) wherein T represents a group represented by T35, R¹ represents C₂F5, and R^{3b} represents any substituents indicated in [Table L4] to [Table L12](hereinafter, referred to as Compound Class SX₂₃₇₀).

A compound (L-5) wherein T represents a group represented by T35, R¹ represents OCF₃, and R^{3b} represents any substituents indicated in [Table L4] to [Table L12](hereinafter, referred to as Compound Class SX₂₃₇₁).

A compound (L-5) wherein T represents a group represented by T35, R¹ represents SCF₃, and R^{3b} represents any substituents indicated in [Table L4] to [Table L12](hereinafter, referred to as Compound Class SX₂₃₇₂).

A compound (L-5) wherein T represents a group represented by T35, R¹ represents S(O)CF₃, and R^{3b} represents any substituents indicated in [Table L4] to [Table L12](hereinafter, referred to as Compound Class SX₂₃₇₃).

A compound (L-5) wherein T represents a group represented by T35, R¹ represents S(O)₂CF₃, and R^{3b} represents any substituents indicated in [Table L4] to [Table L12](hereinafter, referred to as Compound Class SX₂₃₇₄).

A compound (L-5) wherein T represents a group represented by T35, R¹ represents a fluorine atom, and R^{3b} represents any substituents indicated in [Table L4] to [Table L12] (hereinafter, referred to as Compound Class SX₂₃₇₅).

A compound (L-5) wherein T represents a group represented by T35, R¹ represents a chlorine atom, and R^{3b} represents any substituents indicated in [Table L4] to [Table L12] (hereinafter, referred to as Compound Class SX₂₃₇₆).

A compound (L-5) wherein T represents a group represented by T35, R¹ represents a bromine atom, and R^{3b} represents any substituents indicated in [Table L4] to [Table L12] (hereinafter, referred to as Compound Class SX₂₃₇₇).

A compound (L-5) wherein T represents a group represented by T35, R¹ represents an iodine atom, and R^{3b} represents any substituents indicated in [Table L4] to [Table L12] (hereinafter, referred to as Compound Class SX₂₃₇₈).

A compound (L-5) wherein T represents a group represented by T36, R¹ represents CF₃, and R^{3b} represents any substituents indicated in [Table L4] to [Table L12](hereinafter, referred to as Compound Class SX₂₃₇₉).

A compound (L-5) wherein T represents a group represented by T36, R¹ represents C₂F5, and R^{3b} represents any substituents indicated in [Table L4] to [Table L12](hereinafter, referred to as Compound Class SX₂₃₈₀).

A compound (L-5) wherein T represents a group represented by T36, R¹ represents OCF₃, and R^{3b} represents any substituents indicated in [Table L4] to [Table L12](hereinafter, referred to as Compound Class SX₂₃₈₁).

A compound (L-5) wherein T represents a group represented by T36, R¹ represents SCF₃, and R^{3b} represents any substituents indicated in [Table L4] to [Table L12](hereinafter, referred to as Compound Class SX₂₃₈₂).

A compound (L-5) wherein T represents a group represented by T36, R¹ represents S(O)CF₃, and R^{3b} represents any substituents indicated in [Table L4] to [Table L12](hereinafter, referred to as Compound Class SX₂₃₈₃).

A compound (L-5) wherein T represents a group represented by T36, R¹ represents S(O)₂CF₃, and R^{3b} represents any substituents indicated in [Table L4] to [Table L12](hereinafter, referred to as Compound Class SX₂₃₈₄).

A compound (L-5) wherein T represents a group represented by T36, R¹ represents a fluorine atom, and R^{3b} represents any substituents indicated in [Table L4] to [Table L12] (hereinafter, referred to as Compound Class SX₂₃₈₅).

A compound (L-5) wherein T represents a group represented by T34, R¹ represents a chlorine atom, and R^{3b} represents any substituents indicated in [Table L4] to [Table L12] (hereinafter, referred to as Compound Class SX₂₃₈₆).

A compound (L-5) wherein T represents a group represented by T36, R¹ represents a bromine atom, and R^{3b} represents any substituents indicated in [Table L4] to [Table L12] (hereinafter, referred to as Compound Class SX₂₃₈₇).

A compound (L-5) wherein T represents a group represented by T36, R¹ represents an iodine atom, and R^{3b} represents any substituents indicated in [Table L4] to [Table L12] (hereinafter, referred to as Compound Class SX₂₃₈₈).

A compound (L-6) wherein T represents a group represented by T32, R¹ represents CF₃, and R^{3b} represents any substituents indicated in [Table L4] to [Table L12](hereinafter, referred to as Compound Class SX₂₃₈₉).

A compound (L-6) wherein T represents a group represented by T32, R¹ represents C₂F5, and R^{3b} represents any substituents indicated in [Table L4] to [Table L12](hereinafter, referred to as Compound Class SX₂₃₉₀).

A compound (L-6) wherein T represents a group represented by T32, R¹ represents OCF₃, and R^{3b} represents any substituents indicated in [Table L4] to [Table L12](hereinafter, referred to as Compound Class SX₂₃₉₁).

A compound (L-6) wherein T represents a group represented by T32, R¹ represents SCF₃, and R^{3b} represents any substituents indicated in [Table L4] to [Table L12](hereinafter, referred to as Compound Class SX₂₃₉₂).

A compound (L-6) wherein T represents a group represented by T32 R¹ represents S(O)CF₃, and R^{3b} represents any substituents indicated in [Table L4] to [Table L12](hereinafter, referred to as Compound Class SX₂₃₉₃).

A compound (L-6) wherein T represents a group represented by T32, R¹ represents S(O)₂CF₃, and R^{3b} represents any substituents indicated in [Table L4] to [Table L12](hereinafter, referred to as Compound Class SX₂₃₉₄).

A compound (L-6) wherein T represents a group represented by T32, R¹ represents a fluorine atom, and R^{3b} represents any substituents indicated in [Table L4] to [Table L12] (hereinafter, referred to as Compound Class SX₂₃₉₅).

A compound (L-6) wherein T represents a group represented by T32, R¹ represents a chlorine atom, and R^{3b} represents any substituents indicated in [Table L4] to [Table L12] (hereinafter, referred to as Compound Class SX₂₃₉₆).

A compound (L-6) wherein T represents a group represented by T32, R¹ represents a bromine atom, and R^{3b} represents any substituents indicated in [Table L4] to [Table L12] (hereinafter, referred to as Compound Class SX₂₃₉₇).

A compound (L-6) wherein T represents a group represented by T32, R¹ represents an iodine atom, and R^{3b} represents any substituents indicated in [Table L4] to [Table L12] (hereinafter, referred to as Compound Class SX₂₃₉₈).

A compound (L-6) wherein T represents a group represented by T33, R¹ represents CF₃, and R^{3b} represents any substituents indicated in [Table L4] to [Table L12](hereinafter, referred to as Compound Class SX₂₃₉₉).

A compound (L-6) wherein T represents a group represented by T33, R¹ represents C₂F5, and R^{3b} represents any substituents indicated in [Table L4] to [Table L12](hereinafter, referred to as Compound Class SX₂₄₀₀).

A compound (L-6) wherein T represents a group represented by T33, R¹ represents OCF₃, and R^{3b} represents any substituents indicated in [Table L4] to [Table L12](hereinafter, referred to as Compound Class SX₂₄₀₁).

A compound (L-6) wherein T represents a group represented by T33, R¹ represents SCF₃, and R^{3b} represents any substituents indicated in [Table L4] to [Table L12](hereinafter, referred to as Compound Class SX₂₄₀₂).

A compound (L-6) wherein T represents a group represented by T33 R¹ represents S(O)CF₃, and R^{3b} represents any substituents indicated in [Table L4] to [Table L12](hereinafter, referred to as Compound Class SX₂₄₀₃).

A compound (L-6) wherein T represents a group represented by T33, R¹ represents S(O)₂CF₃, and R^{3b} represents any substituents indicated in [Table L4] to [Table L12](hereinafter, referred to as Compound Class SX₂₄₀₄).

A compound (L-6) wherein T represents a group represented by T33, R¹ represents a fluorine atom, and R^{3b} represents any substituents indicated in [Table L4] to [Table L12] (hereinafter, referred to as Compound Class SX₂₄₀₅).

A compound (L-6) wherein T represents a group represented by T33, R¹ represents a chlorine atom, and R^{3b} represents any substituents indicated in [Table L4] to [Table L12] (hereinafter, referred to as Compound Class SX₂₄₀₆).

A compound (L-6) wherein T represents a group represented by T33, R¹ represents a bromine atom, and R^{3b} represents any substituents indicated in [Table L4] to [Table L12] (hereinafter, referred to as Compound Class SX₂₄₀₇).

A compound (L-6) wherein T represents a group represented by T33, R¹ represents an iodine atom, and R^{3b} represents any substituents indicated in [Table L4] to [Table L12] (hereinafter, referred to as Compound Class SX₂₄₀₈).

A compound (L-6) wherein T represents a group represented by T34, R¹ represents CF₃, and R^{3b} represents any substituents indicated in [Table L4] to [Table L12](hereinafter, referred to as Compound Class SX₂₄₀₉).

A compound (L-6) wherein T represents a group represented by T34, R¹ represents C₂F5, and R^{3b} represents any substituents indicated in [Table L4] to [Table L12](hereinafter, referred to as Compound Class SX₂₄₁₀).

A compound (L-6) wherein T represents a group represented by T34, R¹ represents OCF₃, and R^{3b} represents any substituents indicated in [Table L4] to [Table L12](hereinafter, referred to as Compound Class SX₂₄₁₁).

A compound (L-6) wherein T represents a group represented by T34, R¹ represents SCF₃, and R^{3b} represents any substituents indicated in [Table L4] to [Table L12](hereinafter, referred to as Compound Class SX₂₄₁₂).

A compound (L-6) wherein T represents a group represented by T34 R¹ represents S(O)CF₃, and R^{3b} represents any substituents indicated in [Table L4] to [Table L12](hereinafter, referred to as Compound Class SX₂₄₁₃).

A compound (L-6) wherein T represents a group represented by T34, R¹ represents S(O)₂CF₃, and R^{3b} represents any substituents indicated in [Table L4] to [Table L12](hereinafter, referred to as Compound Class SX₂₄₁₄).

A compound (L-6) wherein T represents a group represented by T34, R¹ represents a fluorine atom, and R^{3b} represents any substituents indicated in [Table L4] to [Table L12] (hereinafter, referred to as Compound Class SX₂₄₁₅).

A compound (L-6) wherein T represents a group represented by T34, R¹ represents a chlorine atom, and R^{3b} represents any substituents indicated in [Table L4] to [Table L12] (hereinafter, referred to as Compound Class SX₂₄₁₆).

A compound (L-6) wherein T represents a group represented by T34, R¹ represents a bromine atom, and R^{3b} represents any substituents indicated in [Table L4] to [Table L12] (hereinafter, referred to as Compound Class SX₂₄₁₇).

A compound (L-6) wherein T represents a group represented by T34, R¹ represents an iodine atom, and R^{3b} represents any substituents indicated in [Table L4] to [Table L12] (hereinafter, referred to as Compound Class SX₂₄₁₈).

A compound (L-6) wherein T represents a group represented by T35, R¹ represents CF₃, and R^{3b} represents any substituents indicated in [Table L4] to [Table L12](hereinafter, referred to as Compound Class SX₂₄₁₉).

A compound (L-6) wherein T represents a group represented by T35, R¹ represents C₂F5, and R^{3b} represents any substituents indicated in [Table L4] to [Table L12](hereinafter, referred to as Compound Class SX₂₄₂₀).

A compound (L-6) wherein T represents a group represented by T35, R¹ represents OCF₃, and R^{3b} represents any substituents indicated in [Table L4] to [Table L12](hereinafter, referred to as Compound Class SX₂₄₂₁).

A compound (L-6) wherein T represents a group represented by T35, R¹ represents SCF₃, and R^{3b} represents any substituents indicated in [Table L4] to [Table L12](hereinafter, referred to as Compound Class SX₂₄₂₂).

A compound (L-6) wherein T represents a group represented by T35, R¹ represents S(O)CF₃, and R^{3b} represents any substituents indicated in [Table L4] to [Table L12](hereinafter, referred to as Compound Class SX₂₄₂₃).

A compound (L-6) wherein T represents a group represented by T35, R¹ represents S(O)₂CF₃, and R^{3b} represents any substituents indicated in [Table L4] to [Table L12](hereinafter, referred to as Compound Class SX₂₄₂₄).

A compound (L-6) wherein T represents a group represented by T35, R¹ represents a fluorine atom, and R^{3b} represents any substituents indicated in [Table L4] to [Table L12] (hereinafter, referred to as Compound Class SX₂₄₂₅).

A compound (L-6) wherein T represents a group represented by T35, R¹ represents a chlorine atom, and R^{3b} represents any substituents indicated in [Table L4] to [Table L12] (hereinafter, referred to as Compound Class SX₂₄₂₆).

A compound (L-6) wherein T represents a group represented by T35, R¹ represents a bromine atom, and R^{3b} represents any substituents indicated in [Table L4] to [Table L12] (hereinafter, referred to as Compound Class SX₂₄₂₇).

A compound (L-6) wherein T represents a group represented by T35, R¹ represents an iodine atom, and R^{3b} represents any substituents indicated in [Table L4] to [Table L12] (hereinafter, referred to as Compound Class SX₂₄₂₈).

A compound (L-6) wherein T represents a group represented by T36, R¹ represents CF₃, and R³¹ represents any substituents indicated in [Table L4] to [Table L12](hereinafter, referred to as Compound Class SX₂₄₂₉).

A compound (L-6) wherein T represents a group represented by T36, R¹ represents C₂F5, and R³¹ represents any substituents indicated in [Table L4] to [Table L12](hereinafter, referred to as Compound Class SX₂₄₃₀).

A compound (L-6) wherein T represents a group represented by T36, R¹ represents OCF₃, and R^{3b} represents any substituents indicated in [Table L4] to [Table L12](hereinafter, referred to as Compound Class SX₂₄₃₁).

A compound (L-6) wherein T represents a group represented by T36, R¹ represents SCF₃, and R^{3b} represents any substituents indicated in [Table L4] to [Table L12](hereinafter, referred to as Compound Class SX₂₄₃₂).

A compound (L-6) wherein T represents a group represented by T36, R¹ represents S(O)CF₃, and R^{3b} represents any substituents indicated in [Table L4] to [Table L12](hereinafter, referred to as Compound Class SX₂₄₃₃).

A compound (L-6) wherein T represents a group represented by T36, R¹ represents S(O)₂CF₃, and R^{3b} represents any substituents indicated in [Table L4] to [Table L12](hereinafter, referred to as Compound Class SX₂₄₃₄).

A compound (L-6) wherein T represents a group represented by T36, R¹ represents a fluorine atom, and R^{3b} represents any substituents indicated in [Table L4] to [Table L12] (hereinafter, referred to as Compound Class SX₂₄₃₅).

A compound (L-6) wherein T represents a group represented by T36, R¹ represents a chlorine atom, and R^{3b} represents any substituents indicated in [Table L4] to [Table L12] (hereinafter, referred to as Compound Class SX₂₄₃₆).

A compound (L-6) wherein T represents a group represented by T36, R¹ represents a bromine atom, and R^{3b} represents any substituents indicated in [Table L4] to [Table L12] (hereinafter, referred to as Compound Class SX₂₄₃₇).

A compound (L-6) wherein T represents a group represented by T36, R¹ represents an iodine atom, and R^{3b} represents any substituents indicated in [Table L4] to [Table L12] (hereinafter, referred to as Compound Class SX₂₄₃₈).

A compound (L-1) wherein T represents a group represented by T1, R¹ represents OCF₃, and R^{3b} represents any substituents indicated in [Table L4] to [Table L12](hereinafter, referred to as Compound Class SX₂₄₃₉).

A compound (L-1) wherein T represents a group represented by T1, R¹ represents a fluorine atom, and R^{3b} represents any substituents indicated in [Table L4] to [Table L12] (hereinafter, referred to as Compound Class SX₂₄₄₀).

A compound (L-1) wherein T represents a group represented by T1, R¹ represents a chlorine atom, and R^{3b} represents any substituents indicated in [Table L4] to [Table L12] (hereinafter, referred to as Compound Class SX₂₄₄₁).

A compound (L-1) wherein T represents a group represented by T1, R¹ represents a bromine atom, and R^{3b} represents any substituents indicated in [Table L4] to [Table L12] (hereinafter, referred to as Compound Class SX₂₄₄₂).

A compound (L-1) wherein T represents a group represented by T1, R¹ represents an iodine atom, and R^{3b} represents any substituents indicated in [Table L4] to [Table L12] (hereinafter, referred to as Compound Class SX₂₄₄₃).

A compound (L-1) wherein T represents a group represented by T2, R¹ represents OCF₃, and R^{3b} represents any substituents indicated in [Table L4] to [Table L12](hereinafter, referred to as Compound Class SX₂₄₄₄).

A compound (L-1) wherein T represents a group represented by T2, R¹ represents a fluorine atom, and R^{3b} represents any substituents indicated in [Table L4] to [Table L12] (hereinafter, referred to as Compound Class SX₂₄₄₅).

A compound (L-1) wherein T represents a group represented by T2, R¹ represents a chlorine atom, and R^{3b} represents any substituents indicated in [Table L4] to [Table L12] (hereinafter, referred to as Compound Class SX₂₄₄₆).

A compound (L-1) wherein T represents a group represented by T2, R¹ represents a bromine atom, and R^{3b} represents any substituents indicated in [Table L4] to [Table L12] (hereinafter, referred to as Compound Class SX₂₄₄₇).

A compound (L-1) wherein T represents a group represented by T2, R¹ represents an iodine atom, and R^{3b} represents any substituents indicated in [Table L4] to [Table L12] (hereinafter, referred to as Compound Class SX₂₄₄₈).

A compound (L-1) wherein T represents a group represented by T3, R¹ represents OCF₃, and R^{3b} represents any substituents indicated in [Table L4] to [Table L12](hereinafter, referred to as Compound Class SX₂₄₄₉).

A compound (L-1) wherein T represents a group represented by T3, R¹ represents a fluorine atom, and R^{3b} represents any substituents indicated in [Table L4] to [Table L12] (hereinafter, referred to as Compound Class SX₂₄₅₀).

A compound (L-1) wherein T represents a group represented by T3, R¹ represents a chlorine atom, and R^{3b} represents any substituents indicated in [Table L4] to [Table L12] (hereinafter, referred to as Compound Class SX₂₄₅₁).

A compound (L-1) wherein T represents a group represented by T3, R¹ represents a bromine atom, and R^{3b} represents any substituents indicated in [Table L4] to [Table L12] (hereinafter, referred to as Compound Class SX₂₄₅₂).

A compound (L-1) wherein T represents a group represented by T3, R¹ represents an iodine atom, and R^{3b} represents any substituents indicated in [Table L4] to [Table L12] (hereinafter, referred to as Compound Class SX₂₄₅₃).

A compound (L-1) wherein T represents a group represented by T6, R¹ represents OCF₃, and R^{3b} represents any substituents indicated in [Table L4] to [Table L12](hereinafter, referred to as Compound Class SX₂₄₅₄).

A compound (L-1) wherein T represents a group represented by T6, R¹ represents a fluorine atom, and R^{3b} represents any substituents indicated in [Table L4] to [Table L12] (hereinafter, referred to as Compound Class SX₂₄₅₅).

A compound (L-1) wherein T represents a group represented by T6, R¹ represents a chlorine atom, and R^{3b} represents any substituents indicated in [Table L4] to [Table L12] (hereinafter, referred to as Compound Class SX₂₄₅₆).

A compound (L-1) wherein T represents a group represented by T6, R¹ represents a bromine atom, and R^{3b} represents any substituents indicated in [Table L4] to [Table L12] (hereinafter, referred to as Compound Class SX₂₄₅₇).

A compound (L-1) wherein T represents a group represented by T6, R¹ represents an iodine atom, and R^{3b} represents any substituents indicated in [Table L4] to [Table L12] (hereinafter, referred to as Compound Class SX₂₄₅₈).

A compound (L-1) wherein T represents a group represented by T7, R¹ represents OCF₃, and R^{3b} represents any substituents indicated in [Table L4] to [Table L12](hereinafter, referred to as Compound Class SX₂₄₅₉).

A compound (L-1) wherein T represents a group represented by T7, R¹ represents a fluorine atom, and R^{3b} represents any substituents indicated in [Table L4] to [Table L12] (hereinafter, referred to as Compound Class SX₂₄₆₀).

A compound (L-1) wherein T represents a group represented by T7, R¹ represents a chlorine atom, and R^{3b} represents any substituents indicated in [Table L4] to [Table L12] (hereinafter, referred to as Compound Class SX₂₄₆₁).

A compound (L-1) wherein T represents a group represented by T7, R¹ represents a bromine atom, and R^{3b} represents any substituents indicated in [Table L4] to [Table L12] (hereinafter, referred to as Compound Class SX₂₄₆₂).

A compound (L-1) wherein T represents a group represented by T7, R¹ represents an iodine atom, and R^{3b} represents any substituents indicated in [Table L4] to [Table L12] (hereinafter, referred to as Compound Class SX₂₄₆₃).

A compound (L-1) wherein T represents a group represented by T8, R¹ represents OCF₃, and R^{3b} represents any substituents indicated in [Table L4] to [Table L12](hereinafter, referred to as Compound Class SX₂₄₆₄).

A compound (L-1) wherein T represents a group represented by T8, R¹ represents a fluorine atom, and R^{3b} represents any substituents indicated in [Table L4] to [Table L12] (hereinafter, referred to as Compound Class SX₂₄₆₅).

A compound (L-1) wherein T represents a group represented by T8, R¹ represents a chlorine atom, and R^{3b} represents any substituents indicated in [Table L4] to [Table L12] (hereinafter, referred to as Compound Class SX₂₄₆₆).

A compound (L-1) wherein T represents a group represented by T8, R¹ represents a bromine atom, and R^{3b} represents any substituents indicated in [Table L4] to [Table L12] (hereinafter, referred to as Compound Class SX₂₄₆₇).

A compound (L-1) wherein T represents a group represented by T8, R¹ represents an iodine atom, and R^{3b} represents any substituents indicated in [Table L4] to [Table L12] (hereinafter, referred to as Compound Class SX₂₄₆₈).

A compound (L-1) wherein T represents a group represented by T11, R¹ represents OCF₃, and R^{3b} represents any substituents indicated in [Table L4] to [Table L12](hereinafter, referred to as Compound Class SX₂₄₆₉).

A compound (L-1) wherein T represents a group represented by T11, R¹ represents a fluorine atom, and R^{3b} represents any substituents indicated in [Table L4] to [Table L12] (hereinafter, referred to as Compound Class SX₂₄₇₀).

A compound (L-1) wherein T represents a group represented by T11, R¹ represents a chlorine atom, and R^{3b} represents any substituents indicated in [Table L4] to [Table L12] (hereinafter, referred to as Compound Class SX₂₄₇₁).

A compound (L-1) wherein T represents a group represented by T11, R¹ represents a bromine atom, and R^{3b} represents any substituents indicated in [Table L4] to [Table L12] (hereinafter, referred to as Compound Class SX₂₄₇₂).

A compound (L-1) wherein T represents a group represented by T11, R¹ represents an iodine atom, and R^{3b} represents any substituents indicated in [Table L4] to [Table L12] (hereinafter, referred to as Compound Class SX₂₄₇₃).

A compound (L-1) wherein T represents a group represented by T12, R¹ represents OCF₃, and R^{3b} represents any substituents indicated in [Table L4] to [Table L12](hereinafter, referred to as Compound Class SX₂₄₇₄).

A compound (L-1) wherein T represents a group represented by T11, R¹ represents a fluorine atom, and R^{3b} represents any substituents indicated in [Table L4] to [Table L12] (hereinafter, referred to as Compound Class SX₂₄₇₅).

A compound (L-1) wherein T represents a group represented by T11, R¹ represents a chlorine atom, and R^{3b} represents any substituents indicated in [Table L4] to [Table L12] (hereinafter, referred to as Compound Class SX₂₄₇₆).

A compound (L-1) wherein T represents a group represented by T11, R¹ represents a bromine atom, and R^{3b} represents any substituents indicated in [Table L4] to [Table L12] (hereinafter, referred to as Compound Class SX₂₄₇₇).

A compound (L-1) wherein T represents a group represented by T11, R¹ represents an iodine atom, and R^{3b} represents any substituents indicated in [Table L4] to [Table L12] (hereinafter, referred to as Compound Class SX₂₄₇₈).

A compound (L-1) wherein T represents a group represented by T13, R¹ represents OCF₃, and R^{3b} represents any substituents indicated in [Table L4] to [Table L12](hereinafter, referred to as Compound Class SX₂₄₇₉).

A compound (L-1) wherein T represents a group represented by T13, R¹ represents a fluorine atom, and R^{3b} represents any substituents indicated in [Table L4] to [Table L12] (hereinafter, referred to as Compound Class SX₂₄₈₀).

A compound (L-1) wherein T represents a group represented by T13, R¹ represents a chlorine atom, and R^{3b} represents any substituents indicated in [Table L4] to [Table L12] (hereinafter, referred to as Compound Class SX₂₄₈₁).

A compound (L-1) wherein T represents a group represented by T13, R¹ represents a bromine atom, and R^{3b} represents any substituents indicated in [Table L4] to [Table L12] (hereinafter, referred to as Compound Class SX₂₄₈₂).

A compound (L-1) wherein T represents a group represented by T13, R¹ represents an iodine atom, and R^{3b} represents any substituents indicated in [Table L4] to [Table L12] (hereinafter, referred to as Compound Class SX₂₄₈₃).

A compound (L-2) wherein T represents a group represented by T1, R¹ represents OCF₃, and R^{3b} represents any substituents indicated in [Table L4] to [Table L12](hereinafter, referred to as Compound Class SX₂₄₄).

A compound (L-2) wherein T represents a group represented by T1, R¹ represents a fluorine atom, and R^{3b} represents any substituents indicated in [Table L4] to [Table L12] (hereinafter, referred to as Compound Class SX₂₄₈₅).

A compound (L-2) wherein T represents a group represented by T1, R¹ represents a chlorine atom, and R^{3b} represents any substituents indicated in [Table L4] to [Table L12] (hereinafter, referred to as Compound Class SX₂₄₈₆).

A compound (L-2) wherein T represents a group represented by T1, R¹ represents a bromine atom, and R^{3b} represents any substituents indicated in [Table L4] to [Table L12] (hereinafter, referred to as Compound Class SX₂₄₈₇).

A compound (L-2) wherein T represents a group represented by T1, R¹ represents an iodine atom, and R^{3b} represents any substituents indicated in [Table L4] to [Table L12] (hereinafter, referred to as Compound Class SX₂₄₈₈).

A compound (L-2) wherein T represents a group represented by T2, R¹ represents OCF₃, and R^{3b} represents any substituents indicated in [Table L4] to [Table L12](hereinafter, referred to as Compound Class SX₂₄₈₉).

A compound (L-2) wherein T represents a group represented by T2, R¹ represents a fluorine atom, and R^{3b} represents any substituents indicated in [Table L4] to [Table L12] (hereinafter, referred to as Compound Class SX₂₄₉₀).

A compound (L-2) wherein T represents a group represented by T2, R¹ represents a chlorine atom, and R^{3b} represents any substituents indicated in [Table L4] to [Table L12] (hereinafter, referred to as Compound Class SX₂₄₉₁).

A compound (L-2) wherein T represents a group represented by T2, R¹ represents a bromine atom, and R^{3b} represents any substituents indicated in [Table L4] to [Table L12] (hereinafter, referred to as Compound Class SX₂₄₉₂).

A compound (L-2) wherein T represents a group represented by T2, R¹ represents an iodine atom, and R^{3b} represents any substituents indicated in [Table L4] to [Table L12] (hereinafter, referred to as Compound Class SX₂₄₉₃).

A compound (L-2) wherein T represents a group represented by T3, R¹ represents OCF₃, and R^{3b} represents any substituents indicated in [Table L4] to [Table L12](hereinafter, referred to as Compound Class SX₂₄₉₄).

A compound (L-2) wherein T represents a group represented by T3, R¹ represents a fluorine atom, and R^{3b} represents any substituents indicated in [Table L4] to [Table L12] (hereinafter, referred to as Compound Class SX₂₄₉₅).

A compound (L-2) wherein T represents a group represented by T3, R¹ represents a chlorine atom, and R^{3b} represents any substituents indicated in [Table L4] to [Table L12] (hereinafter, referred to as Compound Class SX₂₄₉₆).

A compound (L-2) wherein T represents a group represented by T3, R¹ represents a bromine atom, and R^{3b} represents any substituents indicated in [Table L4] to [Table L12] (hereinafter, referred to as Compound Class SX₂₄₉₇).

A compound (L-2) wherein T represents a group represented by T3, R¹ represents an iodine atom, and R^{3b} represents any substituents indicated in [Table L4] to [Table L12] (hereinafter, referred to as Compound Class SX₂₄₉₈).

A compound (L-2) wherein T represents a group represented by T6, R¹ represents OCF₃, and R^{3b} represents any substituents indicated in [Table L4] to [Table L12](hereinafter, referred to as Compound Class SX₂₄₉₉).

A compound (L-2) wherein T represents a group represented by T6, R¹ represents a fluorine atom, and R^{3b} represents any substituents indicated in [Table L4] to [Table L12] (hereinafter, referred to as Compound Class SX₂₅₀₀).

A compound (L-2) wherein T represents a group represented by T6, R¹ represents a chlorine atom, and R^{3b} represents any substituents indicated in [Table L4] to [Table L12] (hereinafter, referred to as Compound Class SX₂₅₀₁).

A compound (L-2) wherein T represents a group represented by T6, R¹ represents a bromine atom, and R^{3b} represents any substituents indicated in [Table L4] to [Table L12] (hereinafter, referred to as Compound Class SX₂₅₀₂).

A compound (L-2) wherein T represents a group represented by T6, R¹ represents an iodine atom, and R^{3b} represents any substituents indicated in [Table L4] to [Table L12] (hereinafter, referred to as Compound Class SX₂₅₀₃).

A compound (L-2) wherein T represents a group represented by T7, R¹ represents OCF₃, and R^{3b} represents any substituents indicated in [Table L4] to [Table L12](hereinafter, referred to as Compound Class SX₂₅₀₄).

A compound (L-2) wherein T represents a group represented by T7, R¹ represents a fluorine atom, and R^{3b} represents any substituents indicated in [Table L4] to [Table L12] (hereinafter, referred to as Compound Class SX₂₅₀₅).

A compound (L-2) wherein T represents a group represented by T7, R¹ represents a chlorine atom, and R^{3b} represents any substituents indicated in [Table L4] to [Table L12] (hereinafter, referred to as Compound Class SX₂₅₀₆).

A compound (L-2) wherein T represents a group represented by T7, R¹ represents a bromine atom, and R^{3b} represents any substituents indicated in [Table L4] to [Table L12] (hereinafter, referred to as Compound Class SX₂₅₀₇).

A compound (L-2) wherein T represents a group represented by T7, R¹ represents an iodine atom, and R^{3b} represents any substituents indicated in [Table L4] to [Table L12] (hereinafter, referred to as Compound Class SX₂₅₀₈).

A compound (L-2) wherein T represents a group represented by T8, R¹ represents OCF₃, and R^{3b} represents any substituents indicated in [Table L4] to [Table L12](hereinafter, referred to as Compound Class SX₂₅₀₉).

A compound (L-2) wherein T represents a group represented by T8, R¹ represents a fluorine atom, and R^{3b} represents any substituents indicated in [Table L4] to [Table L12] (hereinafter, referred to as Compound Class SX₂₅₁₀).

A compound (L-2) wherein T represents a group represented by T8, R¹ represents a chlorine atom, and R^{3b} represents any substituents indicated in [Table L4] to [Table L12] (hereinafter, referred to as Compound Class SX₂₅₁₁).

A compound (L-2) wherein T represents a group represented by T8, R¹ represents a bromine atom, and R^{3b} represents any substituents indicated in [Table L4] to [Table L12] (hereinafter, referred to as Compound Class SX₂₅₁₂).

A compound (L-2) wherein T represents a group represented by T8, R¹ represents an iodine atom, and R^{3b} represents any substituents indicated in [Table L4] to [Table L12] (hereinafter, referred to as Compound Class SX₂₅₁₃).

A compound (L-2) wherein T represents a group represented by T11, R¹ represents OCF₃, and R^{3b} represents any substituents indicated in [Table L4] to [Table L12](hereinafter, referred to as Compound Class SX₂₅₁₄).

A compound (L-2) wherein T represents a group represented by T11, R¹ represents a fluorine atom, and R^{3b} represents any substituents indicated in [Table L4] to [Table L12] (hereinafter, referred to as Compound Class SX₂₅₁₅).

A compound (L-2) wherein T represents a group represented by T11, R¹ represents a chlorine atom, and R^{3b} represents any substituents indicated in [Table L4] to [Table L12] (hereinafter, referred to as Compound Class SX₂₅₁₆).

A compound (L-2) wherein T represents a group represented by T11, R¹ represents a bromine atom, and R^{3b} represents any substituents indicated in [Table L4] to [Table L12] (hereinafter, referred to as Compound Class SX₂₅₁₇).

A compound (L-2) wherein T represents a group represented by T11, R¹ represents an iodine atom, and R^{3b} represents any substituents indicated in [Table L4] to [Table L12] (hereinafter, referred to as Compound Class SX₂₅₁₈).

A compound (L-2) wherein T represents a group represented by T12, R¹ represents OCF₃, and R^{3b} represents any substituents indicated in [Table L4] to [Table L12](hereinafter, referred to as Compound Class SX₂₅₁₉).

A compound (L-2) wherein T represents a group represented by T12, R¹ represents a fluorine atom, and R^{3b} represents any substituents indicated in [Table L4] to [Table L12] (hereinafter, referred to as Compound Class SX₂₅₂₀).

A compound (L-2) wherein T represents a group represented by T12, R¹ represents a chlorine atom, and R^{3b} represents any substituents indicated in [Table L4] to [Table L12] (hereinafter, referred to as Compound Class SX₂₅₂₁).

A compound (L-2) wherein T represents a group represented by T12, R¹ represents a bromine atom, and R^{3b} represents any substituents indicated in [Table L4] to [Table L12] (hereinafter, referred to as Compound Class SX₂₅₂₂).

A compound (L-2) wherein T represents a group represented by T12, R¹ represents an iodine atom, and R^{3b} represents any substituents indicated in [Table L4] to [Table L12] (hereinafter, referred to as Compound Class SX₂₅₂₃).

A compound (L-2) wherein T represents a group represented by T13, R¹ represents OCF₃, and R^{3b} represents any substituents indicated in [Table L4] to [Table L12](hereinafter, referred to as Compound Class SX₂₅₂₄).

A compound (L-2) wherein T represents a group represented by T13, R¹ represents a fluorine atom, and R^{3b} represents any substituents indicated in [Table L4] to [Table L12] (hereinafter, referred to as Compound Class SX₂₅₂₅).

A compound (L-2) wherein T represents a group represented by T13, R¹ represents a chlorine atom, and R^{3b} represents any substituents indicated in [Table L4] to [Table L12] (hereinafter, referred to as Compound Class SX₂₅₂₆).

A compound (L-2) wherein T represents a group represented by T13, R¹ represents a bromine atom, and R^{3b} represents any substituents indicated in [Table L4] to [Table L12] (hereinafter, referred to as Compound Class SX₂₅₂₇).

A compound (L-2) wherein T represents a group represented by T13, R¹ represents an iodine atom, and R^{3b} represents any substituents indicated in [Table L4] to [Table L12] (hereinafter, referred to as Compound Class SX₂₅₂₈).

A compound (L-3) wherein T represents a group represented by T1, R¹ represents OCF₃, and R^{3b} represents any substituents indicated in [Table L4] to [Table L12](hereinafter, referred to as Compound Class SX₂₅₂₉).

A compound (L-3) wherein T represents a group represented by T1, R¹ represents a fluorine atom, and R^{3b} represents any substituents indicated in [Table L4] to [Table L12] (hereinafter, referred to as Compound Class SX₂₅₃₀).

A compound (L-3) wherein T represents a group represented by T1, R¹ represents a chlorine atom, and R^{3b} represents any substituents indicated in [Table L4] to [Table L12] (hereinafter, referred to as Compound Class SX₂₅₃₁).

A compound (L-3) wherein T represents a group represented by T1, R¹ represents a bromine atom, and R^{3b} represents any substituents indicated in [Table L4] to [Table L12] (hereinafter, referred to as Compound Class SX₂₅₃₂).

A compound (L-3) wherein T represents a group represented by T1, R¹ represents an iodine atom, and R^{3b} represents any substituents indicated in [Table L4] to [Table L12] (hereinafter, referred to as Compound Class SX₂₅₃₃).

A compound (L-3) wherein T represents a group represented by T2, R¹ represents OCF₃, and R^{3b} represents any substituents indicated in [Table L4] to [Table L12](hereinafter, referred to as Compound Class SX₂₅₃₄).

A compound (L-3) wherein T represents a group represented by T2, R¹ represents a fluorine atom, and R^{3b} represents any substituents indicated in [Table L4] to [Table L12] (hereinafter, referred to as Compound Class SX₂₅₃₅).

A compound (L-3) wherein T represents a group represented by T2, R¹ represents a chlorine atom, and R^{3b} represents any substituents indicated in [Table L4] to [Table L12] (hereinafter, referred to as Compound Class SX₂₅₃₆).

A compound (L-3) wherein T represents a group represented by T2, R¹ represents a bromine atom, and R^{3b} represents any substituents indicated in [Table L4] to [Table L12] (hereinafter, referred to as Compound Class SX₂₅₃₇).

A compound (L-3) wherein T represents a group represented by T2, R¹ represents an iodine atom, and R^{3b} represents any substituents indicated in [Table L4] to [Table L12] (hereinafter, referred to as Compound Class SX₂₅₃₈).

A compound (L-3) wherein T represents a group represented by T3, R¹ represents OCF₃, and R^{3b} represents any substituents indicated in [Table L4] to [Table L12](hereinafter, referred to as Compound Class SX₂₅₃₉).

A compound (L-3) wherein T represents a group represented by T3, R¹ represents a fluorine atom, and R^{3b} represents any substituents indicated in [Table L4] to [Table L12] (hereinafter, referred to as Compound Class SX₂₅₄₀).

A compound (L-3) wherein T represents a group represented by T3, R¹ represents a chlorine atom, and R^{3b} represents any substituents indicated in [Table L4] to [Table L12] (hereinafter, referred to as Compound Class SX₂₅₄₁).

A compound (L-3) wherein T represents a group represented by T3, R¹ represents a bromine atom, and R^{3b} represents any substituents indicated in [Table L4] to [Table L12] (hereinafter, referred to as Compound Class SX₂₅₄₂).

A compound (L-3) wherein T represents a group represented by T3, R¹ represents an iodine atom, and R^{3b} represents any substituents indicated in [Table L4] to [Table L12] (hereinafter, referred to as Compound Class SX₂₅₄₃).

A compound (L-3) wherein T represents a group represented by T6, R¹ represents OCF₃, and R^{3b} represents any substituents indicated in [Table L4] to [Table L12](hereinafter, referred to as Compound Class SX₂₅₄₄).

A compound (L-3) wherein T represents a group represented by T6, R¹ represents a fluorine atom, and R^{3b} represents any substituents indicated in [Table L4] to [Table L12] (hereinafter, referred to as Compound Class SX₂₅₄₅).

A compound (L-3) wherein T represents a group represented by T6, R¹ represents a chlorine atom, and R^{3b} represents any substituents indicated in [Table L4] to [Table L12] (hereinafter, referred to as Compound Class SX₂₅₄₆).

A compound (L-3) wherein T represents a group represented by T6, R¹ represents a bromine atom, and R^{3b} represents any substituents indicated in [Table L4] to [Table L12] (hereinafter, referred to as Compound Class SX₂₅₄₇).

A compound (L-3) wherein T represents a group represented by T6, R¹ represents an iodine atom, and R^{3b} represents any substituents indicated in [Table L4] to [Table L12] (hereinafter, referred to as Compound Class SX₂₅₄₈).

A compound (L-3) wherein T represents a group represented by T7, R¹ represents OCF₃, and R^{3b} represents any substituents indicated in [Table L4] to [Table L12](hereinafter, referred to as Compound Class SX₂₅₄₉).

A compound (L-3) wherein T represents a group represented by T7, R¹ represents a fluorine atom, and R^{3b} represents any substituents indicated in [Table L4] to [Table L12] (hereinafter, referred to as Compound Class SX₂₅₅₀).

A compound (L-3) wherein T represents a group represented by T7, R¹ represents a chlorine atom, and R^{3b} represents any substituents indicated in [Table L4] to [Table L12] (hereinafter, referred to as Compound Class SX₂₅₅₁).

A compound (L-3) wherein T represents a group represented by T7, R¹ represents a bromine atom, and R^{3b} represents any substituents indicated in [Table L4] to [Table L12] (hereinafter, referred to as Compound Class SX₂₅₅₂).

A compound (L-3) wherein T represents a group represented by T7, R¹ represents an iodine atom, and R^{3b} represents any substituents indicated in [Table L4] to [Table L12] (hereinafter, referred to as Compound Class SX₂₅₅₃).

A compound (L-3) wherein T represents a group represented by T8, R¹ represents OCF₃, and R^{3b} represents any substituents indicated in [Table L4] to [Table L12](hereinafter, referred to as Compound Class SX₂₅₅₄).

A compound (L-3) wherein T represents a group represented by T8, R¹ represents a fluorine atom, and R^{3b} represents any substituents indicated in [Table L4] to [Table L12] (hereinafter, referred to as Compound Class SX₂₅₅₅).

A compound (L-3) wherein T represents a group represented by T8, R¹ represents a chlorine atom, and R^{3b} represents any substituents indicated in [Table L4] to [Table L12] (hereinafter, referred to as Compound Class SX₂₅₅₆).

A compound (L-3) wherein T represents a group represented by T8, R¹ represents a bromine atom, and R^{3b} represents any substituents indicated in [Table L4] to [Table L12] (hereinafter, referred to as Compound Class SX₂₅₅₇).

A compound (L-3) wherein T represents a group represented by T8, R¹ represents an iodine atom, and R^{3b} represents any substituents indicated in [Table L4] to [Table L12] (hereinafter, referred to as Compound Class SX₂₅₅₈).

A compound (L-3) wherein T represents a group represented by T11, R¹ represents OCF₃, and R^{3b} represents any substituents indicated in [Table L4] to [Table L12](hereinafter, referred to as Compound Class SX₂₅₅₉).

A compound (L-3) wherein T represents a group represented by T11, R¹ represents a fluorine atom, and R^{3b} represents any substituents indicated in [Table L4] to [Table L12] (hereinafter, referred to as Compound Class SX₂₅₆₀).

A compound (L-3) wherein T represents a group represented by T11, R¹ represents a chlorine atom, and R^{3b} represents any substituents indicated in [Table L4] to [Table L12] (hereinafter, referred to as Compound Class SX₂₅₆₁).

A compound (L-3) wherein T represents a group represented by T11, R¹ represents a bromine atom, and R^{3b} represents any substituents indicated in [Table L4] to [Table L12] (hereinafter, referred to as Compound Class SX₂₅₆₂).

A compound (L-3) wherein T represents a group represented by T11, R¹ represents an iodine atom, and R^{3b} represents any substituents indicated in [Table L4] to [Table L12] (hereinafter, referred to as Compound Class SX₂₅₆₃).

A compound (L-3) wherein T represents a group represented by T12, R¹ represents OCF₃, and R^{3b} represents any substituents indicated in [Table L4] to [Table L12](hereinafter, referred to as Compound Class SX₂₅₆₄).

A compound (L-3) wherein T represents a group represented by T12, R¹ represents a fluorine atom, and R^{3b} represents any substituents indicated in [Table L4] to [Table L12] (hereinafter, referred to as Compound Class SX₂₅₆₅).

A compound (L-3) wherein T represents a group represented by T12, R¹ represents a chlorine atom, and R^{3b} represents any substituents indicated in [Table L4] to [Table L12] (hereinafter, referred to as Compound Class SX₂₅₆₆).

A compound (L-3) wherein T represents a group represented by T12, R¹ represents a bromine atom, and R^{3b} represents any substituents indicated in [Table L4] to [Table L12] (hereinafter, referred to as Compound Class SX₂₅₆₇).

A compound (L-3) wherein T represents a group represented by T12, R¹ represents an iodine atom, and R^{3b} represents any substituents indicated in [Table L4] to [Table L12] (hereinafter, referred to as Compound Class SX₂₅₆₈).

A compound (L-3) wherein T represents a group represented by T13, R¹ represents OCF₃, and R^{3b} represents any substituents indicated in [Table L4] to [Table L12](hereinafter, referred to as Compound Class SX₂₅₆₉).

A compound (L-3) wherein T represents a group represented by T13, R¹ represents a fluorine atom, and R^{3b} represents any substituents indicated in [Table L4] to [Table L12] (hereinafter, referred to as Compound Class SX₂₅₇₀).

A compound (L-3) wherein T represents a group represented by T13, R¹ represents a chlorine atom, and R^{3b} represents any substituents indicated in [Table L4] to [Table L12] (hereinafter, referred to as Compound Class SX₂₅₇₁).

A compound (L-3) wherein T represents a group represented by T13, R¹ represents a bromine atom, and R^{3b} represents any substituents indicated in [Table L4] to [Table L12] (hereinafter, referred to as Compound Class SX₂₅₇₂).

A compound (L-3) wherein T represents a group represented by T13, R¹ represents an iodine atom, and R^{3b} represents any substituents indicated in [Table L4] to [Table L12] (hereinafter, referred to as Compound Class SX₂₅₇₃).

A compound (L-4) wherein T represents a group represented by T1, R¹ represents OCF₃, and R^{3b} represents any substituents indicated in [Table L4] to [Table L12](hereinafter, referred to as Compound Class SX₂₅₇₄).

A compound (L-4) wherein T represents a group represented by T1, R¹ represents a fluorine atom, and R^{3b} represents any substituents indicated in [Table L4] to [Table L12] (hereinafter, referred to as Compound Class SX₂₅₇₅).

A compound (L-4) wherein T represents a group represented by T1, R¹ represents a chlorine atom, and R^{3b} represents any substituents indicated in [Table L4] to [Table L12] (hereinafter, referred to as Compound Class SX₂₅₇₆).

A compound (L-4) wherein T represents a group represented by T1, R¹ represents a bromine atom, and R^{3b} represents any substituents indicated in [Table L4] to [Table L12] (hereinafter, referred to as Compound Class SX₂₅₇₇).

A compound (L-4) wherein T represents a group represented by T1, R¹ represents an iodine atom, and R^{3b} represents any substituents indicated in [Table L4] to [Table L12] (hereinafter, referred to as Compound Class SX₂₅₇₈).

A compound (L-4) wherein T represents a group represented by T2, R¹ represents OCF₃, and R^{3b} represents any substituents indicated in [Table L4] to [Table L12](hereinafter, referred to as Compound Class SX₂₅₇₉).

A compound (L-4) wherein T represents a group represented by T2, R¹ represents a fluorine atom, and R^{3b} represents any substituents indicated in [Table L4] to [Table L12] (hereinafter, referred to as Compound Class SX₂₅₈₀).

A compound (L-4) wherein T represents a group represented by T2, R¹ represents a chlorine atom, and R^{3b} represents any substituents indicated in [Table L4] to [Table L12] (hereinafter, referred to as Compound Class SX₂₅₈₁).

A compound (L-4) wherein T represents a group represented by T2, R¹ represents a bromine atom, and R^{3b} represents any substituents indicated in [Table L4] to [Table L12] (hereinafter, referred to as Compound Class SX₂₅₈₂).

A compound (L-4) wherein T represents a group represented by T2, R¹ represents an iodine atom, and R^{3b} represents any substituents indicated in [Table L4] to [Table L12] (hereinafter, referred to as Compound Class SX₂₅₈₃).

A compound (L-4) wherein T represents a group represented by T3, R¹ represents OCF₃, and R^{3b} represents any substituents indicated in [Table L4] to [Table L12](hereinafter, referred to as Compound Class SX₂₅₈₄).

A compound (L-4) wherein T represents a group represented by T3, R¹ represents a fluorine atom, and R^{3b} represents any substituents indicated in [Table L4] to [Table L12] (hereinafter, referred to as Compound Class SX₂₅₈₅).

A compound (L-4) wherein T represents a group represented by T3, R¹ represents a chlorine atom, and R^{3b} represents any substituents indicated in [Table L4] to [Table L12] (hereinafter, referred to as Compound Class SX₂₅₈₆).

A compound (L-4) wherein T represents a group represented by T3, R¹ represents a bromine atom, and R^{3b} represents any substituents indicated in [Table L4] to [Table L12] (hereinafter, referred to as Compound Class SX₂₅₈₇).

A compound (L-4) wherein T represents a group represented by T3, R¹ represents an iodine atom, and R^{3b} represents any substituents indicated in [Table L4] to [Table L12] (hereinafter, referred to as Compound Class SX₂₅₈₈).

A compound (L-4) wherein T represents a group represented by T6, R¹ represents OCF₃, and R^{3b} represents any substituents indicated in [Table L4] to [Table L12](hereinafter, referred to as Compound Class SX₂₅₈₉).

A compound (L-4) wherein T represents a group represented by T6, R¹ represents a fluorine atom, and R^{3b} represents any substituents indicated in [Table L4] to [Table L12] (hereinafter, referred to as Compound Class SX₂₅₉₀).

A compound (L-4) wherein T represents a group represented by T6, R¹ represents a chlorine atom, and R^{3b} represents any substituents indicated in [Table L4] to [Table L12] (hereinafter, referred to as Compound Class SX₂₅₉₁).

A compound (L-4) wherein T represents a group represented by T6, R¹ represents a bromine atom, and R^{3b} represents any substituents indicated in [Table L4] to [Table L12] (hereinafter, referred to as Compound Class SX₂₅₉₂).

A compound (L-4) wherein T represents a group represented by T6, R¹ represents an iodine atom, and R^{3b} represents any substituents indicated in [Table L4] to [Table L12] (hereinafter, referred to as Compound Class SX₂₅₉₃).

A compound (L-4) wherein T represents a group represented by T7, R¹ represents OCF₃, and R^{3b} represents any substituents indicated in [Table L4] to [Table L12](hereinafter, referred to as Compound Class SX₂₅₉₄).

A compound (L-4) wherein T represents a group represented by T7, R¹ represents a fluorine atom, and R^{3b} represents any substituents indicated in [Table L4] to [Table L12] (hereinafter, referred to as Compound Class SX₂₅₉₅).

A compound (L-4) wherein T represents a group represented by T7, R¹ represents a chlorine atom, and R^{3b} represents any substituents indicated in [Table L4] to [Table L12] (hereinafter, referred to as Compound Class SX₂₅₉₆).

A compound (L-4) wherein T represents a group represented by T7, R¹ represents a bromine atom, and R^{3b} represents any substituents indicated in [Table L4] to [Table L12] (hereinafter, referred to as Compound Class SX₂₅₉₇).

A compound (L-4) wherein T represents a group represented by T7, R¹ represents an iodine atom, and R^{3b} represents any substituents indicated in [Table L4] to [Table L12] (hereinafter, referred to as Compound Class SX₂₅₉₈).

A compound (L-4) wherein T represents a group represented by T8, R¹ represents OCF₃, and R^{3b} represents any substituents indicated in [Table L4] to [Table L12](hereinafter, referred to as Compound Class SX₂₅₉₉).

A compound (L-4) wherein T represents a group represented by T8, R¹ represents a fluorine atom, and R^{3b} represents any substituents indicated in [Table L4] to [Table L12] (hereinafter, referred to as Compound Class SX₂₆₀₀).

A compound (L-4) wherein T represents a group represented by T8, R¹ represents a chlorine atom, and R^{3b} represents any substituents indicated in [Table L4] to [Table L12] (hereinafter, referred to as Compound Class SX₂₆₀₁).

A compound (L-4) wherein T represents a group represented by T8, R¹ represents a bromine atom, and R^{3b} represents any substituents indicated in [Table L4] to [Table L12] (hereinafter, referred to as Compound Class SX₂₆₀₂).

A compound (L-4) wherein T represents a group represented by T8, R¹ represents an iodine atom, and R^{3b} represents any substituents indicated in [Table L4] to [Table L12] (hereinafter, referred to as Compound Class SX₂₆₀₃).

A compound (L-4) wherein T represents a group represented by T11, R¹ represents OCF₃, and R^{3b} represents any substituents indicated in [Table L4] to [Table L12](hereinafter, referred to as Compound Class SX₂₆₀₄).

A compound (L-4) wherein T represents a group represented by T11, R¹ represents a fluorine atom, and R^{3b} represents any substituents indicated in [Table L4] to [Table L12] (hereinafter, referred to as Compound Class SX₂₆₀₅).

A compound (L-4) wherein T represents a group represented by T11, R¹ represents a chlorine atom, and R^{3b} represents any substituents indicated in [Table L4] to [Table L12] (hereinafter, referred to as Compound Class SX₂₆₀₆).

A compound (L-4) wherein T represents a group represented by T11, R¹ represents a bromine atom, and R^{3b} represents any substituents indicated in [Table L4] to [Table L12] (hereinafter, referred to as Compound Class SX₂₆₀₇).

A compound (L-4) wherein T represents a group represented by T11, R¹ represents an iodine atom, and R^{3b} represents any substituents indicated in [Table L4] to [Table L12] (hereinafter, referred to as Compound Class SX₂₆₀₈).

A compound (L-4) wherein T represents a group represented by T12, R¹ represents OCF₃, and R^{3b} represents any substituents indicated in [Table L4] to [Table L12](hereinafter, referred to as Compound Class SX₂₆₀₉).

A compound (L-4) wherein T represents a group represented by T12, R¹ represents a fluorine atom, and R^{3b} represents any substituents indicated in [Table L4] to [Table L12] (hereinafter, referred to as Compound Class SX₂₆₁₀).

A compound (L-4) wherein T represents a group represented by T12, R¹ represents a chlorine atom, and R^{3b} represents any substituents indicated in [Table L4] to [Table L12] (hereinafter, referred to as Compound Class SX₂₆₁₁).

A compound (L-4) wherein T represents a group represented by T12, R¹ represents a bromine atom, and R^{3b} represents any substituents indicated in [Table L4] to [Table L12] (hereinafter, referred to as Compound Class SX₂₆₁₂).

A compound (L-4) wherein T represents a group represented by T12, R¹ represents an iodine atom, and R^{3b} represents any substituents indicated in [Table L4] to [Table L12] (hereinafter, referred to as Compound Class SX₂₆₁₃).

A compound (L-4) wherein T represents a group represented by T13, R¹ represents OCF₃, and R^{3b} represents any substituents indicated in [Table L4] to [Table L12](hereinafter, referred to as Compound Class SX₂₆₁₄).

A compound (L-4) wherein T represents a group represented by T13, R¹ represents a fluorine atom, and R^{3b} represents any substituents indicated in [Table L4] to [Table L12] (hereinafter, referred to as Compound Class SX₂₆₁₅).

A compound (L-4) wherein T represents a group represented by T13, R¹ represents a chlorine atom, and R^{3b} represents any substituents indicated in [Table L4] to [Table L12] (hereinafter, referred to as Compound Class SX₂₆₁₆).

A compound (L-4) wherein T represents a group represented by T13, R¹ represents a bromine atom, and R^{3b} represents any substituents indicated in [Table L4] to [Table L12] (hereinafter, referred to as Compound Class SX₂₆₁₇).

A compound (L-4) wherein T represents a group represented by T13, R¹ represents an iodine atom, and R^{3b} represents any substituents indicated in [Table L4] to [Table L12] (hereinafter, referred to as Compound Class SX₂₆₁₈).

A compound (L-5) wherein T represents a group represented by T1, R¹ represents OCF₃, and R^{3b} represents any substituents indicated in [Table L4] to [Table L12](hereinafter, referred to as Compound Class SX₂₆₁₉).

A compound (L-5) wherein T represents a group represented by T1, R¹ represents a fluorine atom, and R^{3b} represents any substituents indicated in [Table L4] to [Table L12] (hereinafter, referred to as Compound Class SX₂₆₂₀).

A compound (L-5) wherein T represents a group represented by T1, R¹ represents a chlorine atom, and R^{3b} represents any substituents indicated in [Table L4] to [Table L12] (hereinafter, referred to as Compound Class SX₂₆₂₁).

A compound (L-5) wherein T represents a group represented by T1, R¹ represents a bromine atom, and R^{3b} represents any substituents indicated in [Table L4] to [Table L12] (hereinafter, referred to as Compound Class SX₂₆₂₂).

A compound (L-5) wherein T represents a group represented by T1, R¹ represents an iodine atom, and R^{3b} represents any substituents indicated in [Table L4] to [Table L12] (hereinafter, referred to as Compound Class SX₂₆₂₃).

A compound (L-5) wherein T represents a group represented by T2, R¹ represents OCF₃, and R^{3b} represents any substituents indicated in [Table L4] to [Table L12](hereinafter, referred to as Compound Class SX₂₆₂₄).

A compound (L-5) wherein T represents a group represented by T2, R¹ represents a fluorine atom, and R^{3b} represents any substituents indicated in [Table L4] to [Table L12] (hereinafter, referred to as Compound Class SX₂₆₂₅).

A compound (L-5) wherein T represents a group represented by T2, R¹ represents a chlorine atom, and R^{3b} represents any substituents indicated in [Table L4] to [Table L12] (hereinafter, referred to as Compound Class SX₂₆₂₆).

A compound (L-5) wherein T represents a group represented by T2, R¹ represents a bromine atom, and R^{3b} represents any substituents indicated in [Table L4] to [Table L12] (hereinafter, referred to as Compound Class SX₂₆₂₇).

A compound (L-5) wherein T represents a group represented by T2, R¹ represents an iodine atom, and R^{3b} represents any substituents indicated in [Table L4] to [Table L12] (hereinafter, referred to as Compound Class SX₂₆₂₈).

A compound (L-5) wherein T represents a group represented by T3, R¹ represents OCF₃, and R^{3b} represents any substituents indicated in [Table L4] to [Table L12](hereinafter, referred to as Compound Class SX₂₆₂₉).

A compound (L-5) wherein T represents a group represented by T3, R¹ represents a fluorine atom, and R^{3b} represents any substituents indicated in [Table L4] to [Table L12] (hereinafter, referred to as Compound Class SX₂₆₃₀).

A compound (L-5) wherein T represents a group represented by T3, R¹ represents a chlorine atom, and R^{3b} represents any substituents indicated in [Table L4] to [Table L12] (hereinafter, referred to as Compound Class SX₂₆₃₁).

A compound (L-5) wherein T represents a group represented by T3, R¹ represents a bromine atom, and R^{3b} represents any substituents indicated in [Table L4] to [Table L12] (hereinafter, referred to as Compound Class SX₂₆₃₂).

A compound (L-5) wherein T represents a group represented by T3, R¹ represents an iodine atom, and R^{3b} represents any substituents indicated in [Table L4] to [Table L12] (hereinafter, referred to as Compound Class SX₂₆₃₃).

A compound (L-5) wherein T represents a group represented by T6, R¹ represents OCF₃, and R^{3b} represents any substituents indicated in [Table L4] to [Table L12](hereinafter, referred to as Compound Class SX₂₆₃₄).

A compound (L-5) wherein T represents a group represented by T6, R¹ represents a fluorine atom, and R^{3b} represents any substituents indicated in [Table L4] to [Table L12] (hereinafter, referred to as Compound Class SX₂₆₃₅).

A compound (L-5) wherein T represents a group represented by T6, R¹ represents a chlorine atom, and R^{3b} represents any substituents indicated in [Table L4] to [Table L12] (hereinafter, referred to as Compound Class SX₂₆₃₆).

A compound (L-5) wherein T represents a group represented by T6, R¹ represents a bromine atom, and R^{3b} represents any substituents indicated in [Table L4] to [Table L12] (hereinafter, referred to as Compound Class SX₂₆₃₇).

A compound (L-5) wherein T represents a group represented by T6, R¹ represents an iodine atom, and R^{3b} represents any substituents indicated in [Table L4] to [Table L12] (hereinafter, referred to as Compound Class SX₂₆₃₈).

A compound (L-5) wherein T represents a group represented by T7, R¹ represents OCF₃, and R^{3b} represents any substituents indicated in [Table L4] to [Table L12](hereinafter, referred to as Compound Class SX₂₆₃₉).

A compound (L-5) wherein T represents a group represented by T7, R¹ represents a fluorine atom, and R^{3b} represents any substituents indicated in [Table L4] to [Table L12] (hereinafter, referred to as Compound Class SX₂₆₄₀).

A compound (L-5) wherein T represents a group represented by T7, R¹ represents a chlorine atom, and R^{3b} represents any substituents indicated in [Table L4] to [Table L12] (hereinafter, referred to as Compound Class SX₂₆₄₁).

A compound (L-5) wherein T represents a group represented by T7, R¹ represents a bromine atom, and R^{3b} represents any substituents indicated in [Table L4] to [Table L12] (hereinafter, referred to as Compound Class SX₂₆₄₂).

A compound (L-5) wherein T represents a group represented by T7, R¹ represents an iodine atom, and R^{3b} represents any substituents indicated in [Table L4] to [Table L12] (hereinafter, referred to as Compound Class SX₂₆₄₃).

A compound (L-5) wherein T represents a group represented by T8, R¹ represents OCF₃, and R^{3b} represents any substituents indicated in [Table L4] to [Table L12](hereinafter, referred to as Compound Class SX₂₆₄₄).

A compound (L-5) wherein T represents a group represented by T8, R¹ represents a fluorine atom, and R^{3b} represents any substituents indicated in [Table L4] to [Table L12] (hereinafter, referred to as Compound Class SX₂₆₄₅).

A compound (L-5) wherein T represents a group represented by T8, R¹ represents a chlorine atom, and R^{3b} represents any substituents indicated in [Table L4] to [Table L12] (hereinafter, referred to as Compound Class SX₂₆₄₆).

A compound (L-5) wherein T represents a group represented by T8, R¹ represents a bromine atom, and R^{3b} represents any substituents indicated in [Table L4] to [Table L12] (hereinafter, referred to as Compound Class SX₂₆₄₇).

A compound (L-5) wherein T represents a group represented by T8, R¹ represents an iodine atom, and R^{3b} represents any substituents indicated in [Table L4] to [Table L12] (hereinafter, referred to as Compound Class SX₂₆₄₈).

A compound (L-5) wherein T represents a group represented by T11, R¹ represents OCF₃, and R^{3b} represents any substituents indicated in [Table L4] to [Table L12](hereinafter, referred to as Compound Class SX₂₆₄₉).

A compound (L-5) wherein T represents a group represented by T11, R¹ represents a fluorine atom, and R^{3b} represents any substituents indicated in [Table L4] to [Table L12] (hereinafter, referred to as Compound Class SX₂₆₅₀).

A compound (L-5) wherein T represents a group represented by T11, R¹ represents a chlorine atom, and R^{3b} represents any substituents indicated in [Table L4] to [Table L12] (hereinafter, referred to as Compound Class SX₂₆₅₁).

A compound (L-5) wherein T represents a group represented by T11, R¹ represents a bromine atom, and R^{3b} represents any substituents indicated in [Table L4] to [Table L12] (hereinafter, referred to as Compound Class SX₂₆₅₂).

A compound (L-5) wherein T represents a group represented by T11, R¹ represents an iodine atom, and R^{3b} represents any substituents indicated in [Table L4] to [Table L12] (hereinafter, referred to as Compound Class SX₂₆₅₃).

A compound (L-5) wherein T represents a group represented by T12, R¹ represents OCF₃, and R^{3b} represents any substituents indicated in [Table L4] to [Table L12](hereinafter, referred to as Compound Class SX₂₆₅₄).

A compound (L-5) wherein T represents a group represented by T12, R¹ represents a fluorine atom, and R^{3b} represents any substituents indicated in [Table L4] to [Table L12] (hereinafter, referred to as Compound Class SX₂₆₅₅).

A compound (L-5) wherein T represents a group represented by T12, R¹ represents a chlorine atom, and R^{3b} represents any substituents indicated in [Table L4] to [Table L12] (hereinafter, referred to as Compound Class SX₂₆₅₆).

A compound (L-5) wherein T represents a group represented by T12, R¹ represents a bromine atom, and R^{3b} represents any substituents indicated in [Table L4] to [Table L12] (hereinafter, referred to as Compound Class SX₂₆₅₇).

A compound (L-5) wherein T represents a group represented by T12, R¹ represents an iodine atom, and R^{3b} represents any substituents indicated in [Table L4] to [Table L12] (hereinafter, referred to as Compound Class SX₂₆₅₈).

A compound (L-5) wherein T represents a group represented by T13, R¹ represents OCF₃, and R^{3b} represents any substituents indicated in [Table L4] to [Table L12](hereinafter, referred to as Compound Class SX₂₆₅₉).

A compound (L-5) wherein T represents a group represented by T13, R¹ represents a fluorine atom, and R^{3b} represents any substituents indicated in [Table L4] to [Table L12] (hereinafter, referred to as Compound Class SX₂₆₆₀).

A compound (L-5) wherein T represents a group represented by T13, R¹ represents a chlorine atom, and R^{3b} represents any substituents indicated in [Table L4] to [Table L12] (hereinafter, referred to as Compound Class SX₂₆₆₁).

A compound (L-5) wherein T represents a group represented by T13, R¹ represents a bromine atom, and R^{3b} represents any substituents indicated in [Table L4] to [Table L12] (hereinafter, referred to as Compound Class SX₂₆₆₂).

A compound (L-5) wherein T represents a group represented by T13, R¹ represents an iodine atom, and R^{3b} represents any substituents indicated in [Table L4] to [Table L12] (hereinafter, referred to as Compound Class SX₂₆₆₃).

A compound (L-6) wherein T represents a group represented by T1, R¹ represents OCF₃, and R^{3b} represents any substituents indicated in [Table L4] to [Table L12](hereinafter, referred to as Compound Class SX₂₆₆₄).

A compound (L-6) wherein T represents a group represented by T1, R¹ represents a fluorine atom, and R^{3b} represents any substituents indicated in [Table L4] to [Table L12] (hereinafter, referred to as Compound Class SX₂₆₆₅).

A compound (L-6) wherein T represents a group represented by T1, R¹ represents a chlorine atom, and R^{3b} represents any substituents indicated in [Table L4] to [Table L12] (hereinafter, referred to as Compound Class SX₂₆₆₆).

A compound (L-6) wherein T represents a group represented by T1, R¹ represents a bromine atom, and R^{3b} represents any substituents indicated in [Table L4] to [Table L12] (hereinafter, referred to as Compound Class SX₂₆₆₇).

A compound (L-6) wherein T represents a group represented by T1, R¹ represents an iodine atom, and R^{3b} represents any substituents indicated in [Table L4] to [Table L12] (hereinafter, referred to as Compound Class SX₂₆₆₈).

A compound (L-6) wherein T represents a group represented by T2, R¹ represents OCF₃, and R^{3b} represents any substituents indicated in [Table L4] to [Table L12](hereinafter, referred to as Compound Class SX₂₆₆₉).

A compound (L-6) wherein T represents a group represented by T2, R¹ represents a fluorine atom, and R^{3b} represents any substituents indicated in [Table L4] to [Table L12] (hereinafter, referred to as Compound Class SX₂₆₇₀).

A compound (L-6) wherein T represents a group represented by T2, R¹ represents a chlorine atom, and R^{3b} represents any substituents indicated in [Table L4] to [Table L12] (hereinafter, referred to as Compound Class SX₂₆₇₁).

A compound (L-6) wherein T represents a group represented by T2, R¹ represents a bromine atom, and R^{3b} represents any substituents indicated in [Table L4] to [Table L12] (hereinafter, referred to as Compound Class SX₂₆₇₂).

A compound (L-6) wherein T represents a group represented by T2, R¹ represents an iodine atom, and R^{3b} represents any substituents indicated in [Table L4] to [Table L12] (hereinafter, referred to as Compound Class SX₂₆₇₃).

A compound (L-6) wherein T represents a group represented by T3, R¹ represents OCF₃, and R^{3b} represents any substituents indicated in [Table L4] to [Table L12](hereinafter, referred to as Compound Class SX₂₆₇₄).

A compound (L-6) wherein T represents a group represented by T3, R¹ represents a fluorine atom, and R^{3b} represents any substituents indicated in [Table L4] to [Table L12] (hereinafter, referred to as Compound Class SX₂₆₇₅).

A compound (L-6) wherein T represents a group represented by T3, R¹ represents a chlorine atom, and R^{3b} represents any substituents indicated in [Table L4] to [Table L12] (hereinafter, referred to as Compound Class SX₂₆₇₆).

A compound (L-6) wherein T represents a group represented by T3, R¹ represents a bromine atom, and R^{3b} represents any substituents indicated in [Table L4] to [Table L12] (hereinafter, referred to as Compound Class SX₂₆₇₇).

A compound (L-6) wherein T represents a group represented by T3, R¹ represents an iodine atom, and R^{3b} represents any substituents indicated in [Table L4] to [Table L12] (hereinafter, referred to as Compound Class SX₂₆₇₈).

A compound (L-6) wherein T represents a group represented by T6, R¹ represents OCF₃, and R^{3b} represents any substituents indicated in [Table L4] to [Table L12](hereinafter, referred to as Compound Class SX₂₆₇₉).

A compound (L-6) wherein T represents a group represented by T6, R¹ represents a fluorine atom, and R^{3b} represents any substituents indicated in [Table L4] to [Table L12] (hereinafter, referred to as Compound Class SX₂₆₈₀).

A compound (L-6) wherein T represents a group represented by T6, R¹ represents a chlorine atom, and R^{3b} represents any substituents indicated in [Table L4] to [Table L12] (hereinafter, referred to as Compound Class SX₂₆₈₁).

A compound (L-6) wherein T represents a group represented by T6, R¹ represents a bromine atom, and R^{3b} represents any substituents indicated in [Table L4] to [Table L12] (hereinafter, referred to as Compound Class SX₂₆₈₂).

A compound (L-6) wherein T represents a group represented by T6, R¹ represents an iodine atom, and R^{3b} represents any substituents indicated in [Table L4] to [Table L12] (hereinafter, referred to as Compound Class SX₂₆₈₃).

A compound (L-6) wherein T represents a group represented by T7, R¹ represents OCF₃, and R^{3b} represents any substituents indicated in [Table L4] to [Table L12](hereinafter, referred to as Compound Class SX₂₆₄).

A compound (L-6) wherein T represents a group represented by T7, R¹ represents a fluorine atom, and R^{3b} represents any substituents indicated in [Table L4] to [Table L12] (hereinafter, referred to as Compound Class SX₂₆₈₅).

A compound (L-6) wherein T represents a group represented by T7, R¹ represents a chlorine atom, and R^{3b} represents any substituents indicated in [Table L4] to [Table L12] (hereinafter, referred to as Compound Class SX₂₆₈₆).

A compound (L-6) wherein T represents a group represented by T7, R¹ represents a bromine atom, and R^{3b} represents any substituents indicated in [Table L4] to [Table L12] (hereinafter, referred to as Compound Class SX₂₆₈₇).

A compound (L-6) wherein T represents a group represented by T7, R¹ represents an iodine atom, and R^{3b} represents any substituents indicated in [Table L4] to [Table L12] (hereinafter, referred to as Compound Class SX₂₆₈₈).

A compound (L-6) wherein T represents a group represented by T8, R¹ represents OCF₃, and R^{3b} represents any substituents indicated in [Table L4] to [Table L12](hereinafter, referred to as Compound Class SX₂₆₈₉).

A compound (L-6) wherein T represents a group represented by T8, R¹ represents a fluorine atom, and R^{3b} represents any substituents indicated in [Table L4] to [Table L12] (hereinafter, referred to as Compound Class SX₂₆₉₀).

A compound (L-6) wherein T represents a group represented by T8, R¹ represents a chlorine atom, and R^{3b} represents any substituents indicated in [Table L4] to [Table L12] (hereinafter, referred to as Compound Class SX₂₆₉₁).

A compound (L-6) wherein T represents a group represented by T8, R¹ represents a bromine atom, and R^{3b} represents any substituents indicated in [Table L4] to [Table L12] (hereinafter, referred to as Compound Class SX₂₆₉₂).

A compound (L-6) wherein T represents a group represented by T8, R¹ represents an iodine atom, and R^{3b} represents any substituents indicated in [Table L4] to [Table L12] (hereinafter, referred to as Compound Class SX₂₆₉₃).

A compound (L-6) wherein T represents a group represented by T11, R¹ represents OCF₃, and R^{3b} represents any substituents indicated in [Table L4] to [Table L12](hereinafter, referred to as Compound Class SX₂₆₉₄).

A compound (L-6) wherein T represents a group represented by T11, R¹ represents a fluorine atom, and R^{3b} represents any substituents indicated in [Table L4] to [Table L12] (hereinafter, referred to as Compound Class SX₂₆₉₅).

A compound (L-6) wherein T represents a group represented by T11, R¹ represents a chlorine atom, and R^{3b} represents any substituents indicated in [Table L4] to [Table L12] (hereinafter, referred to as Compound Class SX₂₆₉₆).

A compound (L-6) wherein T represents a group represented by T11, R¹ represents a bromine atom, and R^{3b} represents any substituents indicated in [Table L4] to [Table L12] (hereinafter, referred to as Compound Class SX₂₆₉₇).

A compound (L-6) wherein T represents a group represented by T11, R¹ represents an iodine atom, and R^{3b} represents any substituents indicated in [Table L4] to [Table L12] (hereinafter, referred to as Compound Class SX₂₆₉₈).

A compound (L-6) wherein T represents a group represented by T12, R¹ represents OCF₃, and R^{3b} represents any substituents indicated in [Table L4] to [Table L12](hereinafter, referred to as Compound Class SX₂₆₉₉).

A compound (L-6) wherein T represents a group represented by T12, R¹ represents a fluorine atom, and R^{3b} represents any substituents indicated in [Table L4] to [Table L12] (hereinafter, referred to as Compound Class SX₂₇₀₀).

A compound (L-6) wherein T represents a group represented by T12, R¹ represents a chlorine atom, and R^{3b} represents any substituents indicated in [Table L4] to [Table L12] (hereinafter, referred to as Compound Class SX₂₇₀₁).

A compound (L-6) wherein T represents a group represented by T12, R¹ represents a bromine atom, and R^{3b} represents any substituents indicated in [Table L4] to [Table L12] (hereinafter, referred to as Compound Class SX₂₇₀₂).

A compound (L-6) wherein T represents a group represented by T12, R¹ represents an iodine atom, and R^{3b} represents any substituents indicated in [Table L4] to [Table L12] (hereinafter, referred to as Compound Class SX₂₇₀₃).

A compound (L-6) wherein T represents a group represented by T13, R¹ represents OCF₃, and R^{3b} represents any substituents indicated in [Table L4] to [Table L12](hereinafter, referred to as Compound Class SX₂₇₀₄).

A compound (L-6) wherein T represents a group represented by T13, R¹ represents a fluorine atom, and R^{3b} represents any substituents indicated in [Table L4] to [Table L12] (hereinafter, referred to as Compound Class SX₂₇₀₅).

A compound (L-6) wherein T represents a group represented by T13, R¹ represents a chlorine atom, and R^{3b} represents any substituents indicated in [Table L4] to [Table L12] (hereinafter, referred to as Compound Class SX₂₇₀₆).

A compound (L-6) wherein T represents a group represented by T13, R¹ represents a bromine atom, and R^{3b} represents any substituents indicated in [Table L4] to [Table L12] (hereinafter, referred to as Compound Class SX₂₇₀₇).

A compound (L-6) wherein T represents a group represented by T13, R¹ represents an iodine atom, and R^{3b} represents any substituents indicated in [Table L4] to [Table L12] (hereinafter, referred to as Compound Class SX₂₇₀₈).

A compound (L-8) wherein T represents a group represented by T1, R¹ represents a fluorine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₀₉-1).

A compound (L-8) wherein T represents a group represented by T1, R¹ represents a fluorine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₀₉-2).

A compound (L-8) wherein T represents a group represented by T1, R¹ represents a fluorine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₁₀).

A compound (L-8) wherein T represents a group represented by T1, R¹ represents a fluorine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₁₁).

A compound (L-8) wherein T represents a group represented by T1, R¹ represents a fluorine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₁₂).

A compound (L-8) wherein T represents a group represented by T2, R¹ represents a fluorine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₁₃).

A compound (L-8) wherein T represents a group represented by T2, R¹ represents a fluorine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₁₄).

A compound (L-8) wherein T represents a group represented by T2, R¹ represents a fluorine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₁₅).

A compound (L-8) wherein T represents a group represented by T2, R¹ represents a fluorine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₁₆).

A compound (L-8) wherein T represents a group represented by T2, R¹ represents a fluorine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₁₇).

A compound (L-8) wherein T represents a group represented by T3, R¹ represents a fluorine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₁₈).

A compound (L-8) wherein T represents a group represented by T3, R¹ represents a fluorine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₁₉).

A compound (L-8) wherein T represents a group represented by T3, R¹ represents a fluorine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₂₀).

A compound (L-8) wherein T represents a group represented by T3, R¹ represents a fluorine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₂₁).

A compound (L-8) wherein T represents a group represented by T3, R¹ represents a fluorine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₂₂).

A compound (L-8) wherein T represents a group represented by T4, R¹ represents a fluorine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₂₃).

A compound (L-8) wherein T represents a group represented by T4, R¹ represents a fluorine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₂₄).

A compound (L-8) wherein T represents a group represented by T4, R¹ represents a fluorine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₂₅).

A compound (L-8) wherein T represents a group represented by T4, R¹ represents a fluorine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₂₆).

A compound (L-8) wherein T represents a group represented by T4, R¹ represents a fluorine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₂₇).

A compound (L-8) wherein T represents a group represented by T5, R¹ represents a fluorine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₂₈).

A compound (L-8) wherein T represents a group represented by T5, R¹ represents a fluorine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₂₉).

A compound (L-8) wherein T represents a group represented by T5, R¹ represents a fluorine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₃₀).

A compound (L-8) wherein T represents a group represented by T5, R¹ represents a fluorine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₃₁).

A compound (L-8) wherein T represents a group represented by T5, R¹ represents a fluorine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₃₂).

A compound (L-8) wherein T represents a group represented by T1, R¹ represents a chlorine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₃₃).

A compound (L-8) wherein T represents a group represented by T1, R¹ represents a chlorine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₃₄).

A compound (L-8) wherein T represents a group represented by T1, R¹ represents a chlorine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₃₅).

A compound (L-8) wherein T represents a group represented by T1, R¹ represents a chlorine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₃₆).

A compound (L-8) wherein T represents a group represented by T1, R¹ represents a chlorine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₃₇).

A compound (L-8) wherein T represents a group represented by T2, R¹ represents a chlorine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₃₈).

A compound (L-8) wherein T represents a group represented by T2, R¹ represents a chlorine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₃₉).

A compound (L-8) wherein T represents a group represented by T2, R¹ represents a chlorine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₄₀).

A compound (L-8) wherein T represents a group represented by T2, R¹ represents a chlorine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₄₁).

A compound (L-8) wherein T represents a group represented by T2, R¹ represents a chlorine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₄₂).

A compound (L-8) wherein T represents a group represented by T3, R¹ represents a chlorine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₄₃).

A compound (L-8) wherein T represents a group represented by T3, R¹ represents a chlorine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₄₄).

A compound (L-8) wherein T represents a group represented by T3, R¹ represents a chlorine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₄₅).

A compound (L-8) wherein T represents a group represented by T3, R¹ represents a chlorine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₄₆).

A compound (L-8) wherein T represents a group represented by T3, R¹ represents a chlorine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₄₇).

A compound (L-8) wherein T represents a group represented by T4, R¹ represents a chlorine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₄₈).

A compound (L-8) wherein T represents a group represented by T4, R¹ represents a chlorine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₄₉).

A compound (L-8) wherein T represents a group represented by T4, R¹ represents a chlorine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₅₀).

A compound (L-8) wherein T represents a group represented by T4, R¹ represents a chlorine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₅₁).

A compound (L-8) wherein T represents a group represented by T4, R¹ represents a chlorine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₅₂).

A compound (L-8) wherein T represents a group represented by T5, R¹ represents a chlorine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₅₃).

A compound (L-8) wherein T represents a group represented by T5, R¹ represents a chlorine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₅₄).

A compound (L-8) wherein T represents a group represented by T5, R¹ represents a chlorine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₅₅).

A compound (L-8) wherein T represents a group represented by T5, R¹ represents a chlorine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₅₆).

A compound (L-8) wherein T represents a group represented by T5, R¹ represents a chlorine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₅₇).

A compound (L-8) wherein T represents a group represented by T1, R¹ represents a bromine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₅₈).

A compound (L-8) wherein T represents a group represented by T1, R¹ represents a bromine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₅₉).

A compound (L-8) wherein T represents a group represented by T1, R¹ represents a bromine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₆₀).

A compound (L-8) wherein T represents a group represented by T1, R¹ represents a bromine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₆₁).

A compound (L-8) wherein T represents a group represented by T1, R¹ represents a bromine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₆₂).

A compound (L-8) wherein T represents a group represented by T2, R¹ represents a bromine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₆₃).

A compound (L-8) wherein T represents a group represented by T2, R¹ represents a bromine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₆₄).

A compound (L-8) wherein T represents a group represented by T2, R¹ represents a bromine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₆₅).

A compound (L-8) wherein T represents a group represented by T2, R¹ represents a bromine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₆₆).

A compound (L-8) wherein T represents a group represented by T2, R¹ represents a bromine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₆₇).

A compound (L-8) wherein T represents a group represented by T3, R¹ represents a bromine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₆₈).

A compound (L-8) wherein T represents a group represented by T3, R¹ represents a bromine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₆₉).

A compound (L-8) wherein T represents a group represented by T3, R¹ represents a bromine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₇₀).

A compound (L-8) wherein T represents a group represented by T3, R¹ represents a bromine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₇₁).

A compound (L-8) wherein T represents a group represented by T3, R¹ represents a bromine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₇₂).

A compound (L-8) wherein T represents a group represented by T4, R¹ represents a bromine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₇₃).

A compound (L-8) wherein T represents a group represented by T4, R¹ represents a bromine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₇₄).

A compound (L-8) wherein T represents a group represented by T4, R¹ represents a bromine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₇₅).

A compound (L-8) wherein T represents a group represented by T4, R¹ represents a bromine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₇₆).

A compound (L-8) wherein T represents a group represented by T4, R¹ represents a bromine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₇₇).

A compound (L-8) wherein T represents a group represented by T5, R¹ represents a bromine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₇₈).

A compound (L-8) wherein T represents a group represented by T5, R¹ represents a bromine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₇₉).

A compound (L-8) wherein T represents a group represented by T5, R¹ represents a bromine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₈₀).

A compound (L-8) wherein T represents a group represented by T5, R¹ represents a bromine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₈₁).

A compound (L-8) wherein T represents a group represented by T5, R¹ represents a bromine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₈₂).

A compound (L-8) wherein T represents a group represented by T1, R¹ represents an iodine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₈₃).

A compound (L-8) wherein T represents a group represented by T1, R¹ represents an iodine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₈₄).

A compound (L-8) wherein T represents a group represented by T1, R¹ represents an iodine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₈₅).

A compound (L-8) wherein T represents a group represented by T1, R¹ represents an iodine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₈₆).

A compound (L-8) wherein T represents a group represented by T1, R¹ represents an iodine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₈₇).

A compound (L-8) wherein T represents a group represented by T2, R¹ represents an iodine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₈₈).

A compound (L-8) wherein T represents a group represented by T2, R¹ represents an iodine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₈₉).

A compound (L-8) wherein T represents a group represented by T2, R¹ represents an iodine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₉₀).

A compound (L-8) wherein T represents a group represented by T2, R¹ represents an iodine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₉₁).

A compound (L-8) wherein T represents a group represented by T2, R¹ represents an iodine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₉₂).

A compound (L-8) wherein T represents a group represented by T3, R¹ represents an iodine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₉₃).

A compound (L-8) wherein T represents a group represented by T3, R¹ represents an iodine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₉₄).

A compound (L-8) wherein T represents a group represented by T3, R¹ represents an iodine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₇₉₅).

A compound (L-8) wherein T represents a group represented by T3, R¹ represents an iodine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₇₉₆).

A compound (L-8) wherein T represents a group represented by T3, R¹ represents an iodine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₇₉₇).

A compound (L-8) wherein T represents a group represented by T4, R¹ represents an iodine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₇₉₈).

A compound (L-8) wherein T represents a group represented by T4, R¹ represents an iodine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₇₉₉).

A compound (L-8) wherein T represents a group represented by T4, R¹ represents an iodine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₈₀₀).

A compound (L-8) wherein T represents a group represented by T4, R¹ represents an iodine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₈₀₁).

A compound (L-8) wherein T represents a group represented by T4, R¹ represents an iodine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₈₀₂).

A compound (L-8) wherein T represents a group represented by T5, R¹ represents an iodine atom, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₈₀₃).

A compound (L-8) wherein T represents a group represented by T5, R¹ represents an iodine atom, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₈₀₄).

A compound (L-8) wherein T represents a group represented by T5, R¹ represents an iodine atom, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₈₀₅).

A compound (L-8) wherein T represents a group represented by T5, R¹ represents an iodine atom, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₈₀₆).

A compound (L-8) wherein T represents a group represented by T5, R¹ represents an iodine atom, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₈₀₇).

A compound (L-8) wherein T represents a group represented by T1, R¹ represents OCF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₈₀₈).

A compound (L-8) wherein T represents a group represented by T1, R¹ represents OCF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₈₀₉).

A compound (L-8) wherein T represents a group represented by T1, R¹ represents OCF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₈₁₀).

A compound (L-8) wherein T represents a group represented by T1, R¹ represents OCF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₈₁₁).

A compound (L-8) wherein T represents a group represented by T1, R¹ represents OCF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₈₁₂).

A compound (L-8) wherein T represents a group represented by T2, R¹ represents OCF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₈₁₃).

A compound (L-8) wherein T represents a group represented by T2, R¹ represents OCF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₈₁₄).

A compound (L-8) wherein T represents a group represented by T2, R¹ represents OCF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₈₁₅).

A compound (L-8) wherein T represents a group represented by T2, R¹ represents OCF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₈₁₆).

A compound (L-8) wherein T represents a group represented by T2, R¹ represents OCF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₈₁₇).

A compound (L-8) wherein T represents a group represented by T3, R¹ represents OCF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₈₁₈).

A compound (L-8) wherein T represents a group represented by T3, R¹ represents OCF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₈₁₉).

A compound (L-8) wherein T represents a group represented by T3, R¹ represents OCF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₈₂₀).

A compound (L-8) wherein T represents a group represented by T3, R¹ represents OCF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₈₂₁).

A compound (L-8) wherein T represents a group represented by T3, R¹ represents OCF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₈₂₂).

A compound (L-8) wherein T represents a group represented by T4, R¹ represents OCF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₈₂₃).

A compound (L-8) wherein T represents a group represented by T4, R¹ represents OCF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₈₂₄).

A compound (L-8) wherein T represents a group represented by T4, R¹ represents OCF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₈₂₅).

A compound (L-8) wherein T represents a group represented by T4, R¹ represents OCF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₈₂₆).

A compound (L-8) wherein T represents a group represented by T4, R¹ represents OCF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₈₂₇).

A compound (L-8) wherein T represents a group represented by T5, R¹ represents OCF₃, n is 0, R² represents a methyl group, and R^{3b} represents a hydrogen atom, a chlorine atom, CF₃, or any substituents indicated in [Table L4] to [Table L12] (hereinafter, referred to as Compound Class SX₂₈₂₈).

A compound (L-8) wherein T represents a group represented by T5, R¹ represents OCF₃, n is 1, R² represents a methyl group, and R^{3b} represents a hydrogen atom, a chlorine atom, CF₃, or any substituents indicated in [Table L4] to [Table L12] (hereinafter, referred to as Compound Class SX₂₈₂₉).

A compound (L-8) wherein T represents a group represented by T5, R¹ represents OCF₃, n is 2, R² represents a methyl group, and R^{3b} represents a hydrogen atom, a chlorine atom, CF₃, or any substituents indicated in [Table L4] to [Table L12] (hereinafter, referred to as Compound Class SX₂₈₃₀).

A compound (L-8) wherein T represents a group represented by T5, R¹ represents OCF₃, n is 0, R² represents an ethyl group, and R^{3b} represents a hydrogen atom, a chlorine atom, CF₃, or any substituents indicated in [Table L4] to [Table L12] (hereinafter, referred to as Compound Class SX₂₈₃₁).

A compound (L-8) wherein T represents a group represented by T3, R¹ represents OCF₃, n is 1, R² represents an ethyl group, and R^{3b} represents a hydrogen atom, a chlorine atom, CF₃, or any substituents indicated in [Table L4] to [Table L12] (hereinafter, referred to as Compound Class SX₂₈₃₂).

Next, examples of the intermediate compound which is prepared according to the Reference Preparation Examples described in the Examples or the Reference Preparation method described herein are described below. Here T37 to T39 are any groups indicated below.

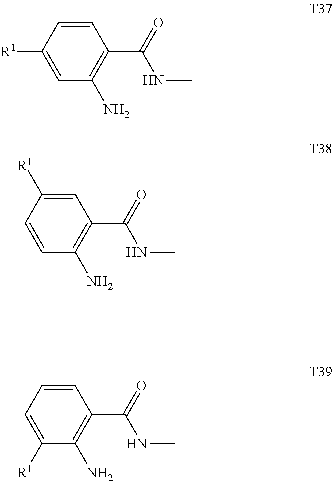

A compound represented by formula (L-9):

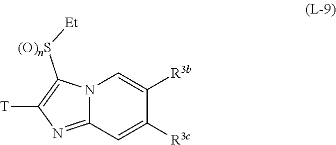

(hereinafter, referred to as Compound (L-9)),
wherein n is 0, T represents a group represented by T37, R^{3b} represents a hydrogen atom, R^{3c} represents a hydrogen atom, and R¹ represents any substituents described in [Table L1] to [Table L3] (hereinafter, referred to as Compound Class SX₂₈₃₃).

A compound (L-9) wherein n is 0, T represents a group represented by T37, R^{3b} represents CF₃, R^{3c} represents a hydrogen atom, and R¹ represents any substituents described in [Table L1] to [Table L3] (hereinafter, referred to as Compound Class SX₂₈₃₄).

A compound (L-9) wherein n is 0, T represents a group represented by T37, R^{3b} represents a chlorine atom, R^{3c} represents a hydrogen atom, and R¹ represents any substituents described in [Table L1] to [Table L3](hereinafter, referred to as Compound Class SX₂₈₃₅).

A compound (L-9) wherein n is 0, T represents a group represented by T37, R^{3b} represents a bromine atom, R^{3c} represents a hydrogen atom, and R¹ represents any substituents described in [Table L1] to [Table L3](hereinafter, referred to as Compound Class SX₂₈₃₆).

A compound (L-9) wherein n is 0, T represents a group represented by T37, R^{3b} represents an iodine atom, R^{3c} represents a hydrogen atom, and R¹ represents any substituents described in [Table L1] to [Table L3](hereinafter, referred to as Compound Class SX₂₈₃₇).

A compound (L-9) wherein n is 0, T represents a group represented by T37, R^{3b} represents a hydrogen atom, R^{3c} represents CF₃, and R¹ represents any substituents described in [Table L1] to [Table L3] (hereinafter, referred to as Compound Class SX₂₈₃₈).

A compound (L-9) wherein n is 0, T represents a group represented by T37, R^{3b} represents a hydrogen atom, R^{3c} represents a chlorine atom, and R¹ represents any substituents described in [Table L1] to [Table L3](hereinafter, referred to as Compound Class SX₂₈₃₉).

A compound (L-9) wherein n is 0, T represents a group represented by T37, R^{3b} represents a hydrogen atom, R^{3c} represents a bromine atom, and R¹ represents any substituents described in [Table L1] to [Table L3] (hereinafter, referred to as Compound Class SX₂₈₄₀).

A compound (L-9) wherein n is 1, T represents a group represented by T37, R^{3b} represents a hydrogen atom, R^{3c} represents a hydrogen atom, and R¹ represents any substituents described in [Table L1] to [Table L3](hereinafter, referred to as Compound Class SX₂₈₄₁).

A compound (L-9) wherein n is 1, T represents a group represented by T37, R^{3b} represents CF₃, R^{3c} represents a hydrogen atom, and R¹ represents any substituents described in [Table L1] to [Table L3] (hereinafter, referred to as Compound Class SX₂₈₄₂).

A compound (L-9) wherein n is 1, T represents a group represented by T37, R^{3b} represents a chlorine atom, R^{3c} represents a hydrogen atom, and R¹ represents any substituents described in [Table L1] to [Table L3](hereinafter, referred to as Compound Class SX₂₈₄₃).

A compound (L-9) wherein n is 1, T represents a group represented by T37, R^{3b} represents a bromine atom, R^{3c} represents a hydrogen atom, and R¹ represents any substituents described in [Table L1] to [Table L3](hereinafter, referred to as Compound Class SX₂₈₄₄).

A compound (L-9) wherein n is 1, T represents a group represented by T37, R^{3b} represents an iodine atom, R^{3c} represents a hydrogen atom, and R¹ represents any substituents described in [Table L1] to [Table L3](hereinafter, referred to as Compound Class SX₂₈₄₅).

A compound (L-9) wherein n is 1, T represents a group represented by T37, R^{3b} represents a hydrogen atom, R^{3c} represents CF₃, and R¹ represents any substituents described in [Table L1] to [Table L3] (hereinafter, referred to as Compound Class SX₂₈₄₆).

A compound (L-9) wherein n is 1, T represents a group represented by T37, R^{3b} represents a hydrogen atom, R^{3c} represents a chlorine atom, and R¹ represents any substituents described in [Table L1] to [Table L3](hereinafter, referred to as Compound Class SX₂₈₄₇).

A compound (L-9) wherein n is 1, T represents a group represented by T37, R^{3b} represents a hydrogen atom, R^{3c} represents a bromine atom, and R¹ represents any substituents described in [Table L1] to [Table L3] (hereinafter, referred to as Compound Class SX₂₈₄₈).

A compound (L-9) wherein n is 2, T represents a group represented by T37, R^{3b} represents a hydrogen atom, R^{3c} represents a hydrogen atom, and R¹ represents any substituents described in [Table L1] to [Table L3](hereinafter, referred to as Compound Class SX₂₈₄₉).

A compound (L-9) wherein n is 2, T represents a group represented by T37, R^{3b} represents CF₃, R^{3c} represents a hydrogen atom, and R¹ represents any substituents described in [Table L1] to [Table L3] (hereinafter, referred to as Compound Class SX₂₈₅₀).

A compound (L-9) wherein n is 2, T represents a group represented by T37, R^{3b} represents a chlorine atom, R^{3c} represents a hydrogen atom, and R¹ represents any substituents described in [Table L1] to [Table L3](hereinafter, referred to as Compound Class SX₂₈₅₁).

A compound (L-9) wherein n is 2, T represents a group represented by T37, R^{3b} represents a bromine atom, R^{3c} represents a hydrogen atom, and R¹ represents any substituents described in [Table L1] to [Table L3](hereinafter, referred to as Compound Class SX₂₈₅₂).

A compound (L-9) wherein n is 2, T represents a group represented by T37, R^{3b} represents an iodine atom, R^{3c} represents a hydrogen atom, and R¹ represents any substituents described in [Table L1] to [Table L3](hereinafter, referred to as Compound Class SX₂₈₅₃).

A compound (L-9) wherein n is 2, T represents a group represented by T37, R^{3b} represents a hydrogen atom, R^{3c} represents CF₃, and R¹ represents any substituents described in [Table L1] to [Table L3] (hereinafter, referred to as Compound Class SX₂₈₅₄).

A compound (L-9) wherein n is 2, T represents a group represented by T37, R^{3b} represents a hydrogen atom, R^{3c} represents a chlorine atom, and R¹ represents any substituents described in [Table L1] to [Table L3](hereinafter, referred to as Compound Class SX₂₈₅₅).

A compound (L-9) wherein n is 2, T represents a group represented by T37, R^{3b} represents a hydrogen atom, R^{3c} represents a bromine atom, and R¹ represents any substituents described in [Table L1] to [Table L3] (hereinafter, referred to as Compound Class SX₂₈₅₆).

A compound (L-9) wherein n is 0, T represents a group represented by T38, R^{3b} represents a hydrogen atom, R^{3c} represents a hydrogen atom, and R¹ represents any substituents described in [Table L1] to [Table L3](hereinafter, referred to as Compound Class SX₂₈₅₇).

A compound (L-9) wherein n is 0, T represents a group represented by T38, R^{3b} represents CF₃, R^{3c} represents a hydrogen atom, and R¹ represents any substituents described in [Table L1] to [Table L3] (hereinafter, referred to as Compound Class SX₂₈₅₈).

A compound (L-9) wherein n is 0, T represents a group represented by T38, R^{3b} represents a chlorine atom, R^{3c} represents a hydrogen atom, and R¹ represents any substituents described in [Table L1] to [Table L3](hereinafter, referred to as Compound Class SX₂₈₅₉).

A compound (L-9) wherein n is 0, T represents a group represented by T38, R^{3b} represents a bromine atom, R^{3c} represents a hydrogen atom, and R¹ represents any substituents described in [Table L1] to [Table L3](hereinafter, referred to as Compound Class SX₂₈₀).

A compound (L-9) wherein n is 0, T represents a group represented by T38, R^{3b} represents an iodine atom, R^{3c} represents a hydrogen atom, and R¹ represents any substituents described in [Table L1] to [Table L3](hereinafter, referred to as Compound Class SX₂₈₆₁).

A compound (L-9) wherein n is 0, T represents a group represented by T38, R^{3b} represents a hydrogen atom, R^{3c} represents CF₃, and R¹ represents any substituents described in [Table L1] to [Table L3] (hereinafter, referred to as Compound Class SX₂₈₆₂).

A compound (L-9) wherein n is 0, T represents a group represented by T38, R^{3b} represents a hydrogen atom, R^{3c} represents a chlorine atom, and R¹ represents any substituents described in [Table L1] to [Table L3](hereinafter, referred to as Compound Class SX₂₈₆₃).

A compound (L-9) wherein n is 0, T represents a group represented by T38, R^{3b} represents a hydrogen atom, R^{3c} represents a bromine atom, and R¹ represents any substituents described in [Table L1] to [Table L3] (hereinafter, referred to as Compound Class SX₂₈₆₄).

A compound (L-9) wherein n is 1, T represents a group represented by T38, R^{3b} represents a hydrogen atom, R^{3c} represents a hydrogen atom, and R¹ represents any substituents described in [Table L1] to [Table L3](hereinafter, referred to as Compound Class SX₂₈₆₅).

A compound (L-9) wherein n is 1, T represents a group represented by T38, R^{3b} represents CF₃, R^{3c} represents a hydrogen atom, and R¹ represents any substituents described in [Table L1] to [Table L3] (hereinafter, referred to as Compound Class SX₂₈₆₆).

A compound (L-9) wherein n is 1, T represents a group represented by T38, R^{3b} represents a chlorine atom, R^{3c} represents a hydrogen atom, and R¹ represents any substituents described in [Table L1] to [Table L3](hereinafter, referred to as Compound Class SX₂₈₆₇).

A compound (L-9) wherein n is 1, T represents a group represented by T38, R^{3b} represents a bromine atom, R^{3c} represents a hydrogen atom, and R¹ represents any substituents described in [Table L1] to [Table L3](hereinafter, referred to as Compound Class SX₂₈₆₈).

A compound (L-9) wherein n is 1, T represents a group represented by T38, R^{3b} represents an iodine atom, R^{3c} represents a hydrogen atom, and R¹ represents any substituents described in [Table L1] to [Table L3](hereinafter, referred to as Compound Class SX₂₈₆₉).

A compound (L-9) wherein n is 1, T represents a group represented by T38, R^{3b} represents a hydrogen atom, R^{3c} represents CF₃, and R¹ represents any substituents described in [Table L1] to [Table L3] (hereinafter, referred to as Compound Class SX₂₈₇₀).

A compound (L-9) wherein n is 1, T represents a group represented by T38, R^{3b} represents a hydrogen atom, R^{3c} represents a chlorine atom, and R¹ represents any substituents described in [Table L1] to [Table L3](hereinafter, referred to as Compound Class SX₂₈₇₁).

A compound (L-9) wherein n is 1, T represents a group represented by T38, R^{3b} represents a hydrogen atom, R^{3c} represents a bromine atom, and R¹ represents any substituents described in [Table L1] to [Table L3] (hereinafter, referred to as Compound Class SX₂₈₇₂).

A compound (L-9) wherein n is 2, T represents a group represented by T38, R^{3b} represents a hydrogen atom, R^{3c} represents a hydrogen atom, and R¹ represents any substituents described in [Table L1] to [Table L3](hereinafter, referred to as Compound Class SX₂₈₇₃).

A compound (L-9) wherein n is 2, T represents a group represented by T38, R^{3b} represents CF₃, R^{3c} represents a hydrogen atom, and R¹ represents any substituents described in [Table L1] to [Table L3] (hereinafter, referred to as Compound Class SX₂₈₇₄).

A compound (L-9) wherein n is 2, T represents a group represented by T38, R^{3b} represents a chlorine atom, R^{3c} represents a hydrogen atom, and R¹ represents any substituents described in [Table L1] to [Table L3](hereinafter, referred to as Compound Class SX₂₈₇₅).

A compound (L-9) wherein n is 2, T represents a group represented by T38, R^{3b} represents a bromine atom, R^{3c} represents a hydrogen atom, and R¹ represents any substituents described in [Table L1] to [Table L3](hereinafter, referred to as Compound Class SX₂₈₇₆).

A compound (L-9) wherein n is 2, T represents a group represented by T38, R^{3b} represents an iodine atom, R^{3c} represents a hydrogen atom, and R¹ represents any substituents described in [Table L1] to [Table L3](hereinafter, referred to as Compound Class SX₂₈₇₇).

A compound (L-9) wherein n is 2, T represents a group represented by T38, R^{3b} represents a hydrogen atom, R^{3c} represents CF₃, and R¹ represents any substituents described in [Table L1] to [Table L3] (hereinafter, referred to as Compound Class SX₂₈₇₈).

A compound (L-9) wherein n is 2, T represents a group represented by T38, R^{3b} represents a hydrogen atom, R^{3c} represents a chlorine atom, and R¹ represents any substituents described in [Table L1] to [Table L3](hereinafter, referred to as Compound Class SX₂₈₇₉).

A compound (L-9) wherein n is 2, T represents a group represented by T38, R^{3b} represents a hydrogen atom, R^{3c} represents a bromine atom, and R¹ represents any substituents described in [Table L1] to [Table L3] (hereinafter, referred to as Compound Class SX₂₈₈₀).

A compound (L-9) wherein n is 0, T represents a group represented by T39, R^{3b} represents a hydrogen atom, R^{3c} represents a hydrogen atom, and R¹ represents any substituents described in [Table L1] to [Table L3](hereinafter, referred to as Compound Class SX₂₈₈₁).

A compound (L-9) wherein n is 0, T represents a group represented by T39, R^{3b} represents CF₃, R^{3c} represents a hydrogen atom, and R¹ represents any substituents described in [Table L1] to [Table L3] (hereinafter, referred to as Compound Class SX₂₈₈₂).

A compound (L-9) wherein n is 0, T represents a group represented by T39, R^{3b} represents a chlorine atom, R^{3c} represents a hydrogen atom, and R¹ represents any substituents described in [Table L1] to [Table L3](hereinafter, referred to as Compound Class SX₂₈₈₃).

A compound (L-9) wherein n is 0, T represents a group represented by T39, R^{3b} represents a bromine atom, R^{3c} represents a hydrogen atom, and R¹ represents any substituents described in [Table L1] to [Table L3](hereinafter, referred to as Compound Class SX₂₈₈₄).

A compound (L-9) wherein n is 0, T represents a group represented by T39, R^{3b} represents an iodine atom, R^{3c} represents a hydrogen atom, and R¹ represents any substituents described in [Table L1] to [Table L3](hereinafter, referred to as Compound Class SX₂₈₈₅).

A compound (L-9) wherein n is 0, T represents a group represented by T39, R^{3b} represents a hydrogen atom, R^{3c} represents CF₃, and R¹ represents any substituents described in [Table L1] to [Table L3] (hereinafter, referred to as Compound Class SX₂₈₈₆).

A compound (L-9) wherein n is 0, T represents a group represented by T39, R^{3b} represents a hydrogen atom, R^{3c} represents a chlorine atom, and R¹ represents any substituents described in [Table L1] to [Table L3](hereinafter, referred to as Compound Class SX₂₈₈₇).

A compound (L-9) wherein n is 0, T represents a group represented by T39, R^{3b} represents a hydrogen atom, R^{3c} represents a bromine atom, and R¹ represents any substituents described in [Table L1] to [Table L3] (hereinafter, referred to as Compound Class SX₂₈₈₈).

A compound (L-9) wherein n is 1, T represents a group represented by T39, R^{3b} represents a hydrogen atom, R^{3c} represents a hydrogen atom, and R¹ represents any substituents described in [Table L1] to [Table L3](hereinafter, referred to as Compound Class SX₂₈₈₉).

A compound (L-9) wherein n is 1, T represents a group represented by T39, R^{3b} represents CF₃, R^{3c} represents a hydrogen atom, and R¹ represents any substituents described in [Table L1] to [Table L3] (hereinafter, referred to as Compound Class SX₂₈₉₀).

A compound (L-9) wherein n is 1, T represents a group represented by T39, R^{3b} represents a chlorine atom, R^{3c} represents a hydrogen atom, and R¹ represents any substituents described in [Table L1] to [Table L3](hereinafter, referred to as Compound Class SX₂₈₉₁).

A compound (L-9) wherein n is 1, T represents a group represented by T39, R^{3b} represents a bromine atom, R^{3c} represents a hydrogen atom, and R¹ represents any substituents described in [Table L1] to [Table L3](hereinafter, referred to as Compound Class SX₂₈₉₂).

A compound (L-9) wherein n is 1, T represents a group represented by T39, R^{3b} represents an iodine atom, R^{3c} represents a hydrogen atom, and R¹ represents any substituents described in [Table L1] to [Table L3](hereinafter, referred to as Compound Class SX₂₈₉₃).

A compound (L-9) wherein n is 1, T represents a group represented by T39, R^{3b} represents a hydrogen atom, R^{3c} represents CF₃, and R¹ represents any substituents described in [Table L1] to [Table L3] (hereinafter, referred to as Compound Class SX₂₈₉₄).

A compound (L-9) wherein n is 1, T represents a group represented by T39, R^{3b} represents a hydrogen atom, R^{3c} represents a chlorine atom, and R¹ represents any substituents described in [Table L1] to [Table L3](hereinafter, referred to as Compound Class SX₂₈₉₅).

A compound (L-9) wherein n is 1, T represents a group represented by T39, R^{3b} represents a hydrogen atom, R^{3c} represents a bromine atom, and R¹ represents any substituents described in [Table L1] to [Table L3] (hereinafter, referred to as Compound Class SX₂₈₉₆).

A compound (L-9) wherein n is 2, T represents a group represented by T39, R^{3b} represents a hydrogen atom, R^{3c} represents a hydrogen atom, and R¹ represents any substituents described in [Table L1] to [Table L3](hereinafter, referred to as Compound Class SX₂₈₉₇).

A compound (L-9) wherein n is 2, T represents a group represented by T39, R^{3b} represents CF₃, R^{3c} represents a hydrogen atom, and R¹ represents any substituents described in [Table L1] to [Table L3] (hereinafter, referred to as Compound Class SX₂₈₉₈).

A compound (L-9) wherein n is 2, T represents a group represented by T39, R^{3b} represents a chlorine atom, R^{3c} represents a hydrogen atom, and R¹ represents any substituents described in [Table L1] to [Table L3](hereinafter, referred to as Compound Class SX₂₈₉₉).

A compound (L-9) wherein n is 2, T represents a group represented by T39, R^{3b} represents a bromine atom, R^{3c} represents a hydrogen atom, and R¹ represents any substituents described in [Table L1] to [Table L3](hereinafter, referred to as Compound Class SX₂₉₀₀).

A compound (L-9) wherein n is 2, T represents a group represented by T39, R^{3b} represents an iodine atom, R^{3c} represents a hydrogen atom, and R¹ represents any substituents described in [Table L1] to [Table L3](hereinafter, referred to as Compound Class SX₂₉₀₁).

A compound (L-9) wherein n is 2, T represents a group represented by T39, R^{3b} represents a hydrogen atom, R^{3c} represents CF₃, and R¹ represents any substituents described in [Table L1] to [Table L3] (hereinafter, referred to as Compound Class SX₂₉₀₂).

A compound (L-9) wherein n is 2, T represents a group represented by T39, R^{3b} represents a hydrogen atom, R^{3c} represents a chlorine atom, and R¹ represents any substituents described in [Table L1] to [Table L3](hereinafter, referred to as Compound Class SX₂₉₀₃).

A compound (L-9) wherein n is 2, T represents a group represented by T39, R^{3b} represents a hydrogen atom, R^{3c} represents a bromine atom, and R¹ represents any substituents described in [Table L1] to [Table L3] (hereinafter, referred to as Compound Class SX₂₉₀₄).

A compound represented by formula (L-10):

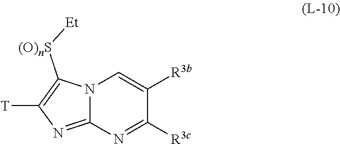

(hereinafter, referred to as Compound (L-10)),
wherein n is 0, T represents T37, R^{3b} represents a hydrogen atom, R^{3c} represents a hydrogen atom, and R¹ represents any substituents described in [Table L1] to [Table L3](hereinafter, referred to as Compound Class SX₂₉₀₅).

A compound (L-10) wherein n is 0, T represents a group represented by T37, R^{3b} represents CF₃, R^{3c} represents a hydrogen atom, and R¹ represents any substituents described in [Table L1] to [Table L3] (hereinafter, referred to as Compound Class SX₂₉₀₆).

A compound (L-10) wherein n is 0, T represents a group represented by T37, R^{3b} represents a chlorine atom, R^{3c} represents a hydrogen atom, and R¹ represents any substituents described in [Table L1] to [Table L3](hereinafter, referred to as Compound Class SX₂₉₀₇).

A compound (L-10) wherein n is 0, T represents a group represented by T37, R^{3b} represents a bromine atom, R^{3c} represents a hydrogen atom, and R¹ represents any substituents described in [Table L1] to [Table L3](hereinafter, referred to as Compound Class SX).

A compound (L-10) wherein n is 0, T represents a group represented by T37, R^{3b} represents an iodine atom, R^{3c} represents a hydrogen atom, and R¹ represents any substituents described in [Table L1] to [Table L3](hereinafter, referred to as Compound Class SX₂₉₀₉).

A compound (L-10) wherein n is 0, T represents a group represented by T37, R^{3b} represents a hydrogen atom, R^{3c} represents CF₃, and R¹ represents any substituents described in [Table L1] to [Table L3] (hereinafter, referred to as Compound Class SX₂₉₁₀).

A compound (L-10) wherein n is 0, T represents a group represented by T37, R^{3b} represents a hydrogen atom, R^{3c} represents a chlorine atom, and R¹ represents any substituents described in [Table L1] to [Table L3](hereinafter, referred to as Compound Class SX₂₉₁₁).

A compound (L-10) wherein n is 0, T represents a group represented by T37, R^{3b} represents a hydrogen atom, R^{3c} represents a bromine atom, and R¹ represents any substituents described in [Table L1] to [Table L3] (hereinafter, referred to as Compound Class SX₂₉₁₂).

A compound (L-10) wherein n is 1, T represents a group represented by T37, R^{3b} represents a hydrogen atom, R^{3c} represents a hydrogen atom, and R¹ represents any substituents described in [Table L1] to [Table L3](hereinafter, referred to as Compound Class SX₂₉₁₃).

A compound (L-10) wherein n is 1, T represents a group represented by T37, R^{3b} represents CF₃, R^{3c} represents a hydrogen atom, and R¹ represents any substituents described in [Table L1] to [Table L3] (hereinafter, referred to as Compound Class SX₂₉₁₄).

A compound (L-10) wherein n is 1, T represents a group represented by T37, R^{3b} represents a chlorine atom, R^{3c} represents a hydrogen atom, and R¹ represents any substituents described in [Table L1] to [Table L3](hereinafter, referred to as Compound Class SX₂₉₁₅).

A compound (L-10) wherein n is 1, T represents a group represented by T37, R^{3b} represents a bromine atom, R^{3c} represents a hydrogen atom, and R¹ represents any substituents described in [Table L1] to [Table L3](hereinafter, referred to as Compound Class SX₂₉₁₆).

A compound (L-10) wherein n is 1, T represents a group represented by T37, R^{3b} represents an iodine atom, R^{3c} represents a hydrogen atom, and R¹ represents any substituents described in [Table L1] to [Table L3](hereinafter, referred to as Compound Class SX₂₉₁₇).

A compound (L-10) wherein n is 1, T represents a group represented by T37, R^{3b} represents a hydrogen atom, R^{3c} represents CF₃, and R¹ represents any substituents described in [Table L1] to [Table L3] (hereinafter, referred to as Compound Class SX₂₉₁₈).

A compound (L-10) wherein n is 1, T represents a group represented by T37, R^{3b} represents a hydrogen atom, R^{3c} represents a chlorine atom, and R¹ represents any substituents described in [Table L1] to [Table L3](hereinafter, referred to as Compound Class SX₂₉₁₉).

A compound (L-10) wherein n is 1, T represents a group represented by T37, R^{3b} represents a hydrogen atom, R^{3c} represents a bromine atom, and R¹ represents any substituents described in [Table L1] to [Table L3] (hereinafter, referred to as Compound Class SX₂₉₂₀).

A compound (L-10) wherein n is 2, T represents a group represented by T37, R^{3b} represents a hydrogen atom, R^{3c} represents a hydrogen atom, and R¹ represents any substituents described in [Table L1] to [Table L3](hereinafter, referred to as Compound Class SX₂₉₂₁).

A compound (L-10) wherein n is 2, T represents a group represented by T37, R^{3b} represents CF₃, R^{3c} represents a hydrogen atom, and R¹ represents any substituents described in [Table L1] to [Table L3] (hereinafter, referred to as Compound Class SX₂₉₂₂).

A compound (L-10) wherein n is 2, T represents a group represented by T37, R^{3b} represents a chlorine atom, R^{3c} represents a hydrogen atom, and R¹ represents any substituents described in [Table L1] to [Table L3](hereinafter, referred to as Compound Class SX₂₉₂₃).

A compound (L-10) wherein n is 2, T represents a group represented by T37, R^{3b} represents a bromine atom, R^{3c} represents a hydrogen atom, and R¹ represents any substituents described in [Table L1] to [Table L3](hereinafter, referred to as Compound Class SX₂₉₂₄).

A compound (L-10) wherein n is 2, T represents a group represented by T37, R^{3b} represents an iodine atom, R^{3c} represents a hydrogen atom, and R¹ represents any substituents described in [Table L1] to [Table L3](hereinafter, referred to as Compound Class SX₂₉₂).

A compound (L-10) wherein n is 2, T represents a group represented by T37, R^{3b} represents a hydrogen atom, R^{3c} represents CF₃, and R¹ represents any substituents described in [Table L1] to [Table L3] (hereinafter, referred to as Compound Class SX₂₉₂₆).

A compound (L-10) wherein n is 2, T represents a group represented by T37, R^{3b} represents a hydrogen atom, R^{3c} represents a chlorine atom, and R¹ represents any substituents described in [Table L1] to [Table L3](hereinafter, referred to as Compound Class SX₂₉₂₇).

A compound (L-10) wherein n is 2, T represents a group represented by T37, R^{3b} represents a hydrogen atom, R^{3c} represents a bromine atom, and R¹ represents any substituents described in [Table L1] to [Table L3] (hereinafter, referred to as Compound Class SX₂₉₂₈).

A compound (L-10) wherein n is 0, T represents a group represented by T38, R^{3b} represents a hydrogen atom, R^{3c} represents a hydrogen atom, and R¹ represents any substituents described in [Table L1] to [Table L3](hereinafter, referred to as Compound Class SX₂₉₂₉).

A compound (L-10) wherein n is 0, T represents a group represented by T38, R^{3b} represents CF₃, R^{3c} represents a hydrogen atom, and R¹ represents any substituents described in [Table L1] to [Table L3] (hereinafter, referred to as Compound Class SX₂₉₃₀).

A compound (L-10) wherein n is 0, T represents a group represented by T38, R^{3b} represents a chlorine atom, R^{3c} represents a hydrogen atom, and R¹ represents any substituents described in [Table L1] to [Table L3](hereinafter, referred to as Compound Class SX₂₉₃₁).

A compound (L-10) wherein n is 0, T represents a group represented by T38, R^{3b} represents a bromine atom, R^{3c} represents a hydrogen atom, and R¹ represents any substituents described in [Table L1] to [Table L3](hereinafter, referred to as Compound Class SX₂₉₃₂).

A compound (L-10) wherein n is 0, T represents a group represented by T38, R^{3b} represents an iodine atom, R^{3c} represents a hydrogen atom, and R¹ represents any substituents described in [Table L1] to [Table L3](hereinafter, referred to as Compound Class SX₂₉₃₃).

A compound (L-10) wherein n is 0, T represents a group represented by T38, R^{3b} represents a hydrogen atom, R^{3c} represents CF₃, and R¹ represents any substituents described in [Table L1] to [Table L3] (hereinafter, referred to as Compound Class SX₂₉₃₄).

A compound (L-10) wherein n is 0, T represents a group represented by T38, R^{3b} represents a hydrogen atom, R^{3c} represents a chlorine atom, and R¹ represents any substituents described in [Table L1] to [Table L3](hereinafter, referred to as Compound Class SX₂₉₃).

A compound (L-10) wherein n is 0, T represents a group represented by T38, R^{3b} represents a hydrogen atom, R^{3c} represents a bromine atom, and R¹ represents any substituents described in [Table L1] to [Table L3] (hereinafter, referred to as Compound Class SX₂₉₃₆).

A compound (L-10) wherein n is 1, T represents a group represented by T38, R^{3b} represents a hydrogen atom, R^{3c} represents a hydrogen atom, and R¹ represents any substituents described in [Table L1] to [Table L3](hereinafter, referred to as Compound Class SX₂₉₃₇).

A compound (L-10) wherein n is 1, T represents a group represented by T38, R^{3b} represents CF₃, R^{3c} represents a hydrogen atom, and R¹ represents any substituents described in [Table L1] to [Table L3] (hereinafter, referred to as Compound Class SX₂₉₃₈).

A compound (L-10) wherein n is 1, T represents a group represented by T38, R^{3b} represents a chlorine atom, R^{3c} represents a hydrogen atom, and R¹ represents any substituents described in [Table L1] to [Table L3](hereinafter, referred to as Compound Class SX₂₉₃₉).

A compound (L-10) wherein n is 1, T represents a group represented by T38, R^{3b} represents a bromine atom, R^{3c} represents a hydrogen atom, and R¹ represents any substituents described in [Table L1] to [Table L3](hereinafter, referred to as Compound Class SX₂₉₄₀).

A compound (L-10) wherein n is 1, T represents a group represented by T38, R^{3b} represents an iodine atom, R^{3c} represents a hydrogen atom, and R¹ represents any substituents described in [Table L1] to [Table L3](hereinafter, referred to as Compound Class SX₂₉₄₁).

A compound (L-10) wherein n is 1, T represents a group represented by T38, R^{3b} represents a hydrogen atom, R^{3c} represents CF₃, and R¹ represents any substituents described in [Table L1] to [Table L3] (hereinafter, referred to as Compound Class SX₂₉₄₂).

A compound (L-10) wherein n is 1, T represents a group represented by T38, R^{3b} represents a hydrogen atom, R^{3c} represents a chlorine atom, and R¹ represents any substituents described in [Table L1] to [Table L3](hereinafter, referred to as Compound Class SX₂₉₄₃).

A compound (L-10) wherein n is 1, T represents a group represented by T38, R^{3b} represents a hydrogen atom, R^{3c} represents a bromine atom, and R¹ represents any substituents described in [Table L1] to [Table L3] (hereinafter, referred to as Compound Class SX₂₉₄₄).

A compound (L-10) wherein n is 2, T represents a group represented by T38, R^{3b} represents a hydrogen atom, R^{3c} represents a hydrogen atom, and R¹ represents any substituents described in [Table L1] to [Table L3](hereinafter, referred to as Compound Class SX₂₉₄₅).

A compound (L-10) wherein n is 2, T represents a group represented by T38, R^{3b} represents CF₃, R^{3c} represents a hydrogen atom, and R¹ represents any substituents described in [Table L1] to [Table L3] (hereinafter, referred to as Compound Class SX₂₉₄₆).

A compound (L-10) wherein n is 2, T represents a group represented by T38, R^{3b} represents a chlorine atom, R^{3c} represents a hydrogen atom, and R¹ represents any substituents described in [Table L1] to [Table L3](hereinafter, referred to as Compound Class SX₂₉₄₇).

A compound (L-10) wherein n is 2, T represents a group represented by T38, R^{3b} represents a bromine atom, R^{3c} represents a hydrogen atom, and R¹ represents any substituents described in [Table L1] to [Table L3](hereinafter, referred to as Compound Class SX₂₉₄₈).

A compound (L-10) wherein n is 2, T represents a group represented by T38, R^{3b} represents an iodine atom, R^{3c} represents a hydrogen atom, and R¹ represents any substituents described in [Table L1] to [Table L3](hereinafter, referred to as Compound Class SX₂₉₄₉).

A compound (L-10) wherein n is 2, T represents a group represented by T38, R^{3b} represents a hydrogen atom, R^{3c} represents CF₃, and R¹ represents any substituents described in [Table L1] to [Table L3] (hereinafter, referred to as Compound Class SX₂₉₅₀).

A compound (L-10) wherein n is 2, T represents a group represented by T38, R^{3b} represents a hydrogen atom, R^{3c} represents a chlorine atom, and R¹ represents any substituents described in [Table L1] to [Table L3](hereinafter, referred to as Compound Class SX₂₉₅₁).

A compound (L-10) wherein n is 2, T represents a group represented by T38, R^{3b} represents a hydrogen atom, R^{3c} represents a bromine atom, and R¹ represents any substituents described in [Table L1] to [Table L3] (hereinafter, referred to as Compound Class SX₂₉₅₂).

A compound (L-10) wherein n is 0, T represents a group represented by T39, R^{3b} represents a hydrogen atom, R^{3c} represents a hydrogen atom, and R¹ represents any substituents described in [Table L1] to [Table L3](hereinafter, referred to as Compound Class SX₂₉₅₃).

A compound (L-10) wherein n is 0, T represents a group represented by T39, R^{3b} represents CF₃, R^{3c} represents a hydrogen atom, and R¹ represents any substituents described in [Table L1] to [Table L3] (hereinafter, referred to as Compound Class SX₂₉₅₄).

A compound (L-10) wherein n is 0, T represents a group represented by T39, R^{3b} represents a chlorine atom, R^{3c} represents a hydrogen atom, and R¹ represents any substituents described in [Table L1] to [Table L3](hereinafter, referred to as Compound Class SX₂₉₅₅).

A compound (L-10) wherein n is 0, T represents a group represented by T39, R^{3b} represents a bromine atom, R^{3c} represents a hydrogen atom, and R¹ represents any substituents described in [Table L1] to [Table L3](hereinafter, referred to as Compound Class SX₂₉₅₆).

A compound (L-10) wherein n is 0, T represents a group represented by T39, R^{3b} represents an iodine atom, R^{3c} represents a hydrogen atom, and R¹ represents any substituents described in [Table L1] to [Table L3](hereinafter, referred to as Compound Class SX₂₉₅₇).

A compound (L-10) wherein n is 0, T represents a group represented by T39, R^{3b} represents a hydrogen atom, R^{3c} represents CF₃, and R¹ represents any substituents described in [Table L1] to [Table L3] (hereinafter, referred to as Compound Class SX₂₉₅₈).

A compound (L-10) wherein n is 0, T represents a group represented by T39, R^{3b} represents a hydrogen atom, R^{3c} represents a chlorine atom, and R¹ represents any substituents described in [Table L1] to [Table L3](hereinafter, referred to as Compound Class SX₂₉₅₉).

A compound (L-10) wherein n is 0, T represents a group represented by T39, R^{3b} represents a hydrogen atom, R^{3c} represents a bromine atom, and R¹ represents any substituents described in [Table L1] to [Table L3] (hereinafter, referred to as Compound Class SX₂₉₆₀).

A compound (L-10) wherein n is 1, T represents a group represented by T39, R^{3b} represents a hydrogen atom, R^{3c} represents a hydrogen atom, and R¹ represents any substituents described in [Table L1] to [Table L3](hereinafter, referred to as Compound Class SX₂₉₆₁).

A compound (L-10) wherein n is 1, T represents a group represented by T39, R^{3b} represents CF₃, R^{3c} represents a hydrogen atom, and R¹ represents any substituents described in [Table L1] to [Table L3] (hereinafter, referred to as Compound Class SX₂₉₆₂).

A compound (L-10) wherein n is 1, T represents a group represented by T39, R^{3b} represents a chlorine atom, R^{3c} represents a hydrogen atom, and R¹ represents any substituents described in [Table L1] to [Table L3](hereinafter, referred to as Compound Class SX₂₉₆₃).

A compound (L-10) wherein n is 1, T represents a group represented by T39, R^{3b} represents a bromine atom, R^{3c} represents a hydrogen atom, and R¹ represents any substituents described in [Table L1] to [Table L3](hereinafter, referred to as Compound Class SX₂₉₆₄).

A compound (L-10) wherein n is 1, T represents a group represented by T39, R^{3b} represents an iodine atom, R^{3c} represents a hydrogen atom, and R¹ represents any substituents described in [Table L1] to [Table L3](hereinafter, referred to as Compound Class SX₂₉₆₅).

A compound (L-10) wherein n is 1, T represents a group represented by T39, R^{3b} represents a hydrogen atom, R^{3c} represents CF₃, and R¹ represents any substituents described in [Table L1] to [Table L3] (hereinafter, referred to as Compound Class SX₂₉₆₆).

A compound (L-10) wherein n is 1, T represents a group represented by T39, R^{3b} represents a hydrogen atom, R^{3c} represents a chlorine atom, and R¹ represents any substituents described in [Table L1] to [Table L3](hereinafter, referred to as Compound Class SX₂₉₆₇).

A compound (L-10) wherein n is 1, T represents a group represented by T39, R^{3b} represents a hydrogen atom, R^{3c} represents a bromine atom, and R¹ represents any substituents described in [Table L1] to [Table L3] (hereinafter, referred to as Compound Class SX₂₉₆₈).

A compound (L-10) wherein n is 2, T represents a group represented by T39, R^{3b} represents a hydrogen atom, R^{3c} represents a hydrogen atom, and R¹ represents any substituents described in [Table L1] to [Table L3](hereinafter, referred to as Compound Class SX₂₉₆₉).

A compound (L-10) wherein n is 2, T represents a group represented by T39, R^{3b} represents CF₃, R^{3c} represents a hydrogen atom, and R¹ represents any substituents described in [Table L1] to [Table L3] (hereinafter, referred to as Compound Class SX₂₉₇₀).

A compound (L-10) wherein n is 2, T represents a group represented by T39, R^{3b} represents a chlorine atom, R^{3c} represents a hydrogen atom, and R¹ represents any substituents described in [Table L1] to [Table L3](hereinafter, referred to as Compound Class SX₂₉₇₁).

A compound (L-10) wherein n is 2, T represents a group represented by T39, R^{3b} represents a bromine atom, R^{3c} represents a hydrogen atom, and R¹ represents any substituents described in [Table L1] to [Table L3](hereinafter, referred to as Compound Class SX₂₉₇₂).

A compound (L-10) wherein n is 2, T represents a group represented by T39, R^{3b} represents an iodine atom, R^{3c} represents a hydrogen atom, and R¹ represents any substituents described in [Table L1] to [Table L3](hereinafter, referred to as Compound Class SX₂₉₇₃).

A compound (L-10) wherein n is 2, T represents a group represented by T39, R^{3b} represents a hydrogen atom, R^{3c} represents CF₃, and R¹ represents any substituents described in [Table L1] to [Table L3] (hereinafter, referred to as Compound Class SX₂₉₇₄).

A compound (L-10) wherein n is 2, T represents a group represented by T39, R^{3b} represents a hydrogen atom, R^{3c} represents a chlorine atom, and R¹ represents any substituents described in [Table L1] to [Table L3](hereinafter, referred to as Compound Class SX₂₉₇₅).

A compound (L-10) wherein n is 2, T represents a group represented by T39, R^{3b} represents a hydrogen atom, R^{3c} represents a bromine atom, and R¹ represents any substituents described in [Table L1] to [Table L3] (hereinafter, referred to as Compound Class SX₂₉₇₆).

A compound (L-9) wherein n is 0, T represents a group represented by T37, R¹ represents CF₃, R^{3c} represents a hydrogen atom, and R^{3b} represents any substituents described in [Table L4] to [Table L12] (hereinafter, referred to as Compound Class SX₂₉₇₇).

A compound (L-9) wherein n is 0, T represents a group represented by T37, R¹ represents C₂F5, R^{3c} represents a hydrogen atom, and R^{3b} represents any substituents described in [Table L4] to [Table L12] (hereinafter, referred to as Compound Class SX₂₉₇₈).

A compound (L-9) wherein n is 0, T represents a group represented by T37, R¹ represents OCF₃, R^{3c} represents a hydrogen atom, and R^{3b} represents any substituents described in [Table L4] to [Table L12] (hereinafter, referred to as Compound Class SX₂₉₇₉).

A compound (L-9) wherein n is 0, T represents a group represented by T37, R¹ represents SCF₃, R^{3c} represents a hydrogen atom, and R^{3b} represents any substituents described in [Table L4] to [Table L12] (hereinafter, referred to as Compound Class SX₂₉₈₀).

A compound (L-9) wherein n is 0, T represents a group represented by T37, R¹ represents S(O)CF₃, R^{3c} represents a hydrogen atom, and R^{3b} represents any substituents described in [Table L4] to [Table L12] (hereinafter, referred to as Compound Class SX₂₉₈₁).

A compound (L-9) wherein n is 0, T represents a group represented by T37, R¹ represents S(O)₂CF₃, R^{3c} represents a hydrogen atom, and R^{3b} represents any substituents described in [Table L4] to [Table L12] (hereinafter, referred to as Compound Class SX₂₉₈₂).

A compound (L-9) wherein n is 1, T represents a group represented by T37, R¹ represents CF₃, R^{3c} represents a hydrogen atom, and R^{3b} represents any substituents described in [Table L4] to [Table L12] (hereinafter, referred to as Compound Class SX₂₉₈₃).

A compound (L-9) wherein n is 1, T represents a group represented by T37, R¹ represents C₂F5, R^{3c} represents a hydrogen atom, and R^{3b} represents any substituents described in [Table L4] to [Table L12] (hereinafter, referred to as Compound Class SX₂₉₈₄).

A compound (L-9) wherein n is 1, T represents a group represented by T37, R¹ represents OCF₃, R^{3c} represents a hydrogen atom, and R^{3b} represents any substituents described in [Table L4] to [Table L12] (hereinafter, referred to as Compound Class SX₂₉₈₅).

A compound (L-9) wherein n is 1, T represents a group represented by T37, R¹ represents SCF₃, R^{3c} represents a hydrogen atom, and R^{3b} represents any substituents described in [Table L4] to [Table L12] (hereinafter, referred to as Compound Class SX₂₉₈₆).

A compound (L-9) wherein n is 1, T represents a group represented by T37, R¹ represents S(O)CF₃, R^{3c} represents a hydrogen atom, and R^{3b} represents any substituents described in [Table L4] to [Table L12] (hereinafter, referred to as Compound Class SX₂₉₈₇).

A compound (L-9) wherein n is 1, T represents a group represented by T37, R¹ represents S(O)₂CF₃, R^{3c} represents a hydrogen atom, and R^{3b} represents any substituents described in [Table L4] to [Table L12] (hereinafter, referred to as Compound Class SX₂₉₈₈).

A compound (L-9) wherein n is 2, T represents a group represented by T37, R¹ represents CF₃, R^{3c} represents a hydrogen atom, and R^{3b} represents any substituents described in [Table L4] to [Table L12] (hereinafter, referred to as Compound Class SX₂₉₈₉).

A compound (L-9) wherein n is 2, T represents a group represented by T37, R¹ represents C₂F5, R^{3c} represents a hydrogen atom, and R^{3b} represents any substituents described in [Table L4] to [Table L12] (hereinafter, referred to as Compound Class SX₂₉₉₀).

A compound (L-9) wherein n is 2, T represents a group represented by T37, R¹ represents OCF₃, R^{3c} represents a hydrogen atom, and R^{3b} represents any substituents described in [Table L4] to [Table L12] (hereinafter, referred to as Compound Class SX₂₉₉₁).

A compound (L-9) wherein n is 2, T represents a group represented by T37, R¹ represents SCF₃, R^{3c} represents a hydrogen atom, and R^{3b} represents any substituents described in [Table L4] to [Table L12] (hereinafter, referred to as Compound Class SX₂₉₉₂).

A compound (L-9) wherein n is 2, T represents a group represented by T37, R¹ represents S(O)CF₃, R^{3c} represents a hydrogen atom, and R^{3b} represents any substituents described in [Table L4] to [Table L12] (hereinafter, referred to as Compound Class SX₂₉₉₃).

A compound (L-9) wherein n is 2, T represents a group represented by T37, R¹ represents S(O)₂CF₃, R^{3c} represents a hydrogen atom, and R^{3b} represents any substituents described in [Table L4] to [Table L12] (hereinafter, referred to as Compound Class SX₂₉₉₄).

A compound (L-9) wherein n is 0, T represents a group represented by T38, R¹ represents CF₃, R^{3c} represents a hydrogen atom, and R^{3b} represents any substituents described in [Table L4] to [Table L12] (hereinafter, referred to as Compound Class SX₂₉₉₅).

A compound (L-9) wherein n is 0, T represents a group represented by T38, R¹ represents C₂F5, R^{3c} represents a hydrogen atom, and R^{3b} represents any substituents described in [Table L4] to [Table L12] (hereinafter, referred to as Compound Class SX₂₉₉₆).

A compound (L-9) wherein n is 0, T represents a group represented by T38, R¹ represents OCF₃, R^{3c} represents a hydrogen atom, and R^{3b} represents any substituents described in [Table L4] to [Table L12] (hereinafter, referred to as Compound Class SX₂₉₉₇).

A compound (L-9) wherein n is 0, T represents a group represented by T38, R¹ represents SCF₃, R^{3c} represents a hydrogen atom, and R^{3b} represents any substituents described in [Table L4] to [Table L12] (hereinafter, referred to as Compound Class SX₂₉₉₈).

A compound (L-9) wherein n is 0, T represents a group represented by T38, R¹ represents S(O)CF₃, R^{3c} represents a hydrogen atom, and R^{3b} represents any substituents described in [Table L4] to [Table L12] (hereinafter, referred to as Compound Class SX₂₉₉₉).

A compound (L-9) wherein n is 0, T represents a group represented by T38, R¹ represents S(O)₂CF₃, R^{3c} represents a hydrogen atom, and R^{3b} represents any substituents described in [Table L4] to [Table L12] (hereinafter, referred to as Compound Class SX₃₀₀₀).

A compound (L-9) wherein n is 1, T represents a group represented by T38, R¹ represents CF₃, R^{3c} represents a hydrogen atom, and R^{3b} represents any substituents described in [Table L4] to [Table L12] (hereinafter, referred to as Compound Class SX₃₀₀₁).

A compound (L-9) wherein n is 1, T represents a group represented by T38, R¹ represents C₂F5, R^{3c} represents a hydrogen atom, and R^{3b} represents any substituents described in [Table L4] to [Table L12] (hereinafter, referred to as Compound Class SX₃₀₀₂).

A compound (L-9) wherein n is 1, T represents a group represented by T38, R¹ represents OCF₃, R^{3c} represents a hydrogen atom, and R^{3b} represents any substituents described in [Table L4] to [Table L12] (hereinafter, referred to as Compound Class SX₃₀₀₃).

A compound (L-9) wherein n is 1, T represents a group represented by T38, R¹ represents SCF₃, R^{3c} represents a hydrogen atom, and R^{3b} represents any substituents described in [Table L4] to [Table L12] (hereinafter, referred to as Compound Class SX₃₀₀₄).

A compound (L-9) wherein n is 1, T represents a group represented by T38, R¹ represents S(O)CF₃, R^{3c} represents a hydrogen atom, and R^{3b} represents any substituents described in [Table L4] to [Table L12] (hereinafter, referred to as Compound Class SX₃₀₀₅).

A compound (L-9) wherein n is 1, T represents a group represented by T38, R¹ represents S(O)₂CF₃, R^{3c} represents a hydrogen atom, and R^{3b} represents any substituents described in [Table L4] to [Table L12] (hereinafter, referred to as Compound Class SX₃₀₀₆).

A compound (L-9) wherein n is 2, T represents a group represented by T38, R¹ represents CF₃, R^{3c} represents a hydrogen atom, and R^{3b} represents any substituents described in [Table L4] to [Table L12] (hereinafter, referred to as Compound Class SX₃₀₀₇).

A compound (L-9) wherein n is 2, T represents a group represented by T38, R¹ represents C₂F5, R^{3c} represents a hydrogen atom, and R^{3b} represents any substituents described in [Table L4] to [Table L12] (hereinafter, referred to as Compound Class SX₃₀₀₈).

A compound (L-9) wherein n is 2, T represents a group represented by T38, R¹ represents OCF₃, R^{3c} represents a hydrogen atom, and R^{3b} represents any substituents described in [Table L4] to [Table L12] (hereinafter, referred to as Compound Class SX₃₀₀₉).

A compound (L-9) wherein n is 2, T represents a group represented by T38, R¹ represents SCF₃, R^{3c} represents a hydrogen atom, and R^{3b} represents any substituents described in [Table L4] to [Table L12] (hereinafter, referred to as Compound Class SX₃₀₁₀).

A compound (L-9) wherein n is 2, T represents a group represented by T38, R¹ represents S(O)CF₃, R^{3c} represents a hydrogen atom, and R^{3b} represents any substituents described in [Table L4] to [Table L12] (hereinafter, referred to as Compound Class SX₃₀₁₁).

A compound (L-9) wherein n is 2, T represents a group represented by T38, R¹ represents S(O)₂CF₃, R^{3c} represents a hydrogen atom, and R^{3b} represents any substituents described in [Table L4] to [Table L12] (hereinafter, referred to as Compound Class SX₃₀₁₂).

A compound (L-9) wherein n is 0, T represents a group represented by T39, R¹ represents CF₃, R^{3c} represents a hydrogen atom, and R^{3b} represents any substituents described in [Table L4] to [Table L12] (hereinafter, referred to as Compound Class SX₃₀₁₃).

A compound (L-9) wherein n is 0, T represents a group represented by T39, R¹ represents C₂F5, R^{3c} represents a hydrogen atom, and R^{3b} represents any substituents described in [Table L4] to [Table L12] (hereinafter, referred to as Compound Class SX₃₀₁₄).

A compound (L-9) wherein n is 0, T represents a group represented by T39, R¹ represents OCF₃, R^{3c} represents a hydrogen atom, and R^{3b} represents any substituents described in [Table L4] to [Table L12] (hereinafter, referred to as Compound Class SX₃₀₁₅).

A compound (L-9) wherein n is 0, T represents a group represented by T39, R¹ represents SCF₃, R^{3c} represents a hydrogen atom, and R^{3b} represents any substituents described in [Table L4] to [Table L12] (hereinafter, referred to as Compound Class SX₃₀₁₆).

A compound (L-9) wherein n is 0, T represents a group represented by T39, R¹ represents S(O)CF₃, R^{3c} represents a hydrogen atom, and R^{3b} represents any substituents described in [Table L4] to [Table L12] (hereinafter, referred to as Compound Class SX₃₀₁₇).

A compound (L-9) wherein n is 0, T represents a group represented by T39, R¹ represents S(O)₂CF₃, R^{3c} represents a hydrogen atom, and R^{3b} represents any substituents described in [Table L4] to [Table L12] (hereinafter, referred to as Compound Class SX₃₀₁₈).

A compound (L-9) wherein n is 1, T represents a group represented by T39, R¹ represents CF₃, R^{3c} represents a hydrogen atom, and R^{3b} represents any substituents described in [Table L4] to [Table L12] (hereinafter, referred to as Compound Class SX₃₀₁₉).

A compound (L-9) wherein n is 1, T represents a group represented by T39, R¹ represents C₂F5, R^{3c} represents a hydrogen atom, and R^{3b} represents any substituents described in [Table L4] to [Table L12] (hereinafter, referred to as Compound Class SX₃₀₂₀).

A compound (L-9) wherein n is 1, T represents a group represented by T39, R¹ represents OCF₃, R^{3c} represents a hydrogen atom, and R^{3b} represents any substituents described in [Table L4] to [Table L12] (hereinafter, referred to as Compound Class SX₃₀₂₁).

A compound (L-9) wherein n is 1, T represents a group represented by T39, R¹ represents SCF₃, R^{3c} represents a hydrogen atom, and R^{3b} represents any substituents described in [Table L4] to [Table L12] (hereinafter, referred to as Compound Class SX₃₀₂₂).

A compound (L-9) wherein n is 1, T represents a group represented by T39, R¹ represents S(O)CF₃, R^{3c} represents a hydrogen atom, and R^{3b} represents any substituents described in [Table L4] to [Table L12] (hereinafter, referred to as Compound Class SX₃₀₂₃).

A compound (L-9) wherein n is 1, T represents a group represented by T39, R¹ represents S(O)₂CF₃, R^{3c} represents a hydrogen atom, and R^{3b} represents any substituents described in [Table L4] to [Table L12] (hereinafter, referred to as Compound Class SX₃₀₂₄).

A compound (L-9) wherein n is 2, T represents a group represented by T39, R¹ represents CF₃, R^{3c} represents a hydrogen atom, and R^{3b} represents any substituents described in [Table L4] to [Table L12] (hereinafter, referred to as Compound Class SX₃₀₂₅).

A compound (L-9) wherein n is 2, T represents a group represented by T39, R¹ represents C₂F5, R^{3c} represents a hydrogen atom, and R^{3b} represents any substituents described in [Table L4] to [Table L12] (hereinafter, referred to as Compound Class SX₃₀₂₆).

A compound (L-9) wherein n is 2, T represents a group represented by T39, R¹ represents OCF₃, R^{3c} represents a hydrogen atom, and R^{3b} represents any substituents described in [Table L4] to [Table L12] (hereinafter, referred to as Compound Class SX₃₀₂₇).

A compound (L-9) wherein n is 2, T represents a group represented by T39, R¹ represents SCF₃, R^{3c} represents a hydrogen atom, and R^{3b} represents any substituents described in [Table L4] to [Table L12] (hereinafter, referred to as Compound Class SX₃₀₂₈).

A compound (L-9) wherein n is 2, T represents a group represented by T39, R¹ represents S(O)CF₃, R^{3c} represents a hydrogen atom, and R^{3b} represents any substituents described in [Table L4] to [Table L12] (hereinafter, referred to as Compound Class SX₃₀₂₉).

A compound (L-9) wherein n is 2, T represents a group represented by T39, R¹ represents S(O)₂CF₃, R^{3c} represents a hydrogen atom, and R^{3b} represents any substituents described in [Table L4] to [Table L12] (hereinafter, referred to as Compound Class SX₃₀₃₀).

A compound (L-10) wherein n is 0, T represents a group represented by T37, R¹ represents CF₃, R^{3c} represents a hydrogen atom, and R^{3b} represents any substituents described in [Table L4] to [Table L12] (hereinafter, referred to as Compound Class SX₃₀₃₁).

A compound (L-10) wherein n is 0, T represents a group represented by T37, R¹ represents C₂F5, R^{3c} represents a hydrogen atom, and R^{3b} represents any substituents described in [Table L4] to [Table L12] (hereinafter, referred to as Compound Class SX₃₀₃₂).

A compound (L-10) wherein n is 0, T represents a group represented by T37, R¹ represents OCF₃, R^{3c} represents a hydrogen atom, and R^{3b} represents any substituents described in [Table L4] to [Table L12] (hereinafter, referred to as Compound Class SX₃₀₃₃).

A compound (L-10) wherein n is 0, T represents a group represented by T37, R¹ represents SCF₃, R^{3c} represents a hydrogen atom, and R^{3b} represents any substituents described in [Table L4] to [Table L12] (hereinafter, referred to as Compound Class SX₃₀₃₄).

A compound (L-10) wherein n is 0, T represents a group represented by T37, R¹ represents S(O)CF₃, R^{3c} represents a hydrogen atom, and R^{3b} represents any substituents described in [Table L4] to [Table L12] (hereinafter, referred to as Compound Class SX₃₀₃₅).

A compound (L-10) wherein n is 0, T represents a group represented by T37, R¹ represents S(O)₂CF₃, R^{3c} represents a hydrogen atom, and R^{3b} represents any substituents described in [Table L4] to [Table L12] (hereinafter, referred to as Compound Class SX₃₀₃₆).

A compound (L-10) wherein n is 1, T represents a group represented by T37, R¹ represents CF₃, R^{3c} represents a hydrogen atom, and R^{3b} represents any substituents described in [Table L4] to [Table L12] (hereinafter, referred to as Compound Class SX₃₀₃₇).

A compound (L-10) wherein n is 1, T represents a group represented by T37, R¹ represents C₂F5, R^{3c} represents a hydrogen atom, and R^{3b} represents any substituents described in [Table L4] to [Table L12] (hereinafter, referred to as Compound Class SX₃₀₃₈).

A compound (L-10) wherein n is 1, T represents a group represented by T37, R¹ represents OCF₃, R^{3c} represents a hydrogen atom, and R^{3b} represents any substituents described in [Table L4] to [Table L12] (hereinafter, referred to as Compound Class SX₃₀₃₉).

A compound (L-10) wherein n is 1, T represents a group represented by T37, R¹ represents SCF₃, R^{3c} represents a hydrogen atom, and R^{3b} represents any substituents described in [Table L4] to [Table L12] (hereinafter, referred to as Compound Class SX₃₀₄₀).

A compound (L-10) wherein n is 1, T represents a group represented by T37, R¹ represents S(O)CF₃, R^{3c} represents a hydrogen atom, and R^{3b} represents any substituents described in [Table L4] to [Table L12] (hereinafter, referred to as Compound Class SX₃₀₄₁).

A compound (L-10) wherein n is 1, T represents a group represented by T37, R¹ represents S(O)₂CF₃, R^{3c} represents a hydrogen atom, and R^{3b} represents any substituents described in [Table L4] to [Table L12] (hereinafter, referred to as Compound Class SX₃₀₄₂).

A compound (L-10) wherein n is 2, T represents a group represented by T37, R¹ represents CF₃, R^{3c} represents a hydrogen atom, and R^{3b} represents any substituents described in [Table L4] to [Table L12] (hereinafter, referred to as Compound Class SX₃₀₄₃).

A compound (L-10) wherein n is 2, T represents a group represented by T37, R¹ represents C₂F5, R^{3c} represents a hydrogen atom, and R^{3b} represents any substituents described in [Table L4] to [Table L12] (hereinafter, referred to as Compound Class SX₃₀₄₄).

A compound (L-10) wherein n is 2, T represents a group represented by T37, R¹ represents OCF₃, R^{3c} represents a hydrogen atom, and R^{3b} represents any substituents described in [Table L4] to [Table L12] (hereinafter, referred to as Compound Class SX₃₀₄₅).

A compound (L-10) wherein n is 2, T represents a group represented by T37, R¹ represents SCF₃, R^{3c} represents a hydrogen atom, and R^{3b} represents any substituents described in [Table L4] to [Table L12] (hereinafter, referred to as Compound Class SX₃₀₄₆).

A compound (L-10) wherein n is 2, T represents a group represented by T37, R¹ represents S(O)CF₃, R^{3c} represents a hydrogen atom, and R^{3b} represents any substituents described in [Table L4] to [Table L12] (hereinafter, referred to as Compound Class SX₃₀₄₇).

A compound (L-10) wherein n is 0, T represents a group represented by T37, R¹ represents S(O)₂CF₃, R^{3c} represents a hydrogen atom, and R^{3b} represents any substituents described in [Table L4] to [Table L12] (hereinafter, referred to as Compound Class SX₃₀₄₈).

A compound (L-10) wherein n is 0, T represents a group represented by T38, R¹ represents CF₃, R^{3c} represents a hydrogen atom, and R^{3b} represents any substituents described in [Table L4] to [Table L12] (hereinafter, referred to as Compound Class SX₃₀₄₉).

A compound (L-10) wherein n is 0, T represents a group represented by T38, R¹ represents C₂F5, R^{3c} represents a hydrogen atom, and R^{3b} represents any substituents described in [Table L4] to [Table L12] (hereinafter, referred to as Compound Class SX₃₀₅₀).

A compound (L-10) wherein n is 0, T represents a group represented by T38, R¹ represents OCF₃, R^{3c} represents a hydrogen atom, and R^{3b} represents any substituents described in [Table L4] to [Table L12] (hereinafter, referred to as Compound Class SX₃₀₅₁).

A compound (L-10) wherein n is 0, T represents a group represented by T38, R¹ represents SCF₃, R^{3c} represents a hydrogen atom, and R^{3b} represents any substituents described in [Table L4] to [Table L12] (hereinafter, referred to as Compound Class SX₃₀₅₂).

A compound (L-10) wherein n is 0, T represents a group represented by T38, R¹ represents S(O)CF₃, R^{3c} represents a hydrogen atom, and R^{3b} represents any substituents described in [Table L4] to [Table L12] (hereinafter, referred to as Compound Class SX₃₀₅₃).

A compound (L-10) wherein n is 0, T represents a group represented by T38, R¹ represents S(O)₂CF₃, R^{3c} represents a hydrogen atom, and R^{3b} represents any substituents described in [Table L4] to [Table L12] (hereinafter, referred to as Compound Class SX₃₀₅₄).

A compound (L-10) wherein n is 1, T represents a group represented by T38, R¹ represents CF₃, R^{3c} represents a hydrogen atom, and R^{3b} represents any substituents described in [Table L4] to [Table L12] (hereinafter, referred to as Compound Class SX₃₀₅₅).

A compound (L-10) wherein n is 1, T represents a group represented by T38, R¹ represents C₂F5, R^{3c} represents a hydrogen atom, and R^{3b} represents any substituents described in [Table L4] to [Table L12] (hereinafter, referred to as Compound Class SX₃₀₅₆).

A compound (L-10) wherein n is 1, T represents a group represented by T38, R¹ represents OCF₃, R^{3c} represents a hydrogen atom, and R^{3b} represents any substituents described in [Table L4] to [Table L12] (hereinafter, referred to as Compound Class SX₃₀₅₇).

A compound (L-10) wherein n is 1, T represents a group represented by T38, R¹ represents SCF₃, R^{3c} represents a hydrogen atom, and R^{3b} represents any substituents described in [Table L4] to [Table L12] (hereinafter, referred to as Compound Class SX₃₀₅₈).

A compound (L-10) wherein n is 1, T represents a group represented by T38, R¹ represents S(O)CF₃, R^{3c} represents a hydrogen atom, and R^{3b} represents any substituents described in [Table L4] to [Table L12] (hereinafter, referred to as Compound Class SX₃₀₅₉).

A compound (L-10) wherein n is 1, T represents a group represented by T38, R¹ represents S(O)₂CF₃, R^{3c} represents a hydrogen atom, and R^{3b} represents any substituents described in [Table L4] to [Table L12] (hereinafter, referred to as Compound Class SX₃₀₆₀).

A compound (L-10) wherein n is 2, T represents a group represented by T38, R¹ represents CF₃, R^{3c} represents a hydrogen atom, and R^{3b} represents any substituents described in [Table L4] to [Table L12] (hereinafter, referred to as Compound Class SX₃₀₆₁).

A compound (L-10) wherein n is 2, T represents a group represented by T38, R¹ represents C₂F5, R^{3c} represents a hydrogen atom, and R^{3b} represents any substituents described in [Table L4] to [Table L12] (hereinafter, referred to as Compound Class SX₃₀₆₂).

A compound (L-10) wherein n is 2, T represents a group represented by T38, R¹ represents OCF₃, R^{3c} represents a hydrogen atom, and R^{3b} represents any substituents described in [Table L4] to [Table L12] (hereinafter, referred to as Compound Class SX₃₀₆₃).

A compound (L-10) wherein n is 2, T represents a group represented by T38, R¹ represents SCF₃, R^{3c} represents a hydrogen atom, and R^{3b} represents any substituents described in [Table L4] to [Table L12] (hereinafter, referred to as Compound Class SX₃₀₆₄).

A compound (L-10) wherein n is 2, T represents a group represented by T38, R¹ represents S(O)CF₃, R^{3c} represents a hydrogen atom, and R^{3b} represents any substituents described in [Table L4] to [Table L12] (hereinafter, referred to as Compound Class SX₃₀₆₅).

A compound (L-10) wherein n is 2, T represents a group represented by T38, R¹ represents S(O)₂CF₃, R^{3c} represents a hydrogen atom, and R^{3b} represents any substituents described in [Table L4] to [Table L12] (hereinafter, referred to as Compound Class SX₃₀₆₆).

A compound (L-10) wherein n is 0, T represents a group represented by T39, R¹ represents CF₃, R^{3c} represents a hydrogen atom, and R^{3b} represents any substituents described in [Table L4] to [Table L12] (hereinafter, referred to as Compound Class SX₃₀₆₇).

A compound (L-10) wherein n is 0, T represents a group represented by T39, R¹ represents C₂F5, R^{3c} represents a hydrogen atom, and R^{3b} represents any substituents described in [Table L4] to [Table L12] (hereinafter, referred to as Compound Class SX₃₀₆₈).

A compound (L-10) wherein n is 0, T represents a group represented by T39, R¹ represents OCF₃, R^{3c} represents a hydrogen atom, and R^{3b} represents any substituents described in [Table L4] to [Table L12] (hereinafter, referred to as Compound Class SX₃₀₆₉).

A compound (L-10) wherein n is 0, T represents a group represented by T39, R¹ represents SCF₃, R^{3c} represents a hydrogen atom, and R^{3b} represents any substituents described in [Table L4] to [Table L12] (hereinafter, referred to as Compound Class SX₃₀₇₀).

A compound (L-10) wherein n is 0, T represents a group represented by T39, R¹ represents S(O)CF₃, R^{3c} represents a hydrogen atom, and R^{3b} represents any substituents described in [Table L4] to [Table L12] (hereinafter, referred to as Compound Class SX₃₀₇₁).

A compound (L-10) wherein n is 0, T represents a group represented by T39, R¹ represents S(O)₂CF₃, R^{3c} represents a hydrogen atom, and R^{3b} represents any substituents described in [Table L4] to [Table L12] (hereinafter, referred to as Compound Class SX₃₀₇₂).

A compound (L-10) wherein n is 1, T represents a group represented by T39, R¹ represents CF₃, R^{3c} represents a hydrogen atom, and R^{3b} represents any substituents described in [Table L4] to [Table L12] (hereinafter, referred to as Compound Class SX₃₀₇₃).

A compound (L-10) wherein n is 1, T represents a group represented by T39, R¹ represents C₂F5, R^{3c} represents a hydrogen atom, and R^{3b} represents any substituents described in [Table L4] to [Table L12] (hereinafter, referred to as Compound Class SX₃₀₇₄).

A compound (L-10) wherein n is 1, T represents a group represented by T39, R¹ represents OCF₃, R^{3c} represents a hydrogen atom, and R^{3b} represents any substituents described in [Table L4] to [Table L12] (hereinafter, referred to as Compound Class SX₃₀₇₅).

A compound (L-10) wherein n is 1 T represents a group represented by T39, R¹ represents SCF₃, R^{3c} represents a hydrogen atom, and R^{3b} represents any substituents described in [Table L4] to [Table L12] (hereinafter, referred to as Compound Class SX₃₀₇₆).

A compound (L-10) wherein n is 1, T represents a group represented by T39, R¹ represents S(O)CF₃, R^{3c} represents a hydrogen atom, and R^{3b} represents any substituents described in [Table L4] to [Table L12] (hereinafter, referred to as Compound Class SX₃₀₇₇).

A compound (L-10) wherein n is 1, T represents a group represented by T39, R¹ represents S(O)₂CF₃, R^{3c} represents a hydrogen atom, and R^{3b} represents any substituents described in [Table L4] to [Table L12] (hereinafter, referred to as Compound Class SX₃₀₇₈).

A compound (L-10) wherein n is 2, T represents a group represented by T39, R¹ represents CF₃, R^{3c} represents a hydrogen atom, and R^{3b} represents any substituents described in [Table L4] to [Table L12] (hereinafter, referred to as Compound Class SX₃₀₇₉).

A compound (L-10) wherein n is 2, T represents a group represented by T39, R¹ represents C₂F5, R^{3c} represents a hydrogen atom, and R^{3b} represents any substituents described in [Table L4] to [Table L12] (hereinafter, referred to as Compound Class SX₃₀₈₀).

A compound (L-10) wherein n is 2, T represents a group represented by T39, R¹ represents OCF₃, R^{3c} represents a hydrogen atom, and R^{3b} represents any substituents described in [Table L4] to [Table L12] (hereinafter, referred to as Compound Class SX₃₀₈₁).

A compound (L-10) wherein n is 2, T represents a group represented by T39, R¹ represents SCF₃, R^{3c} represents a hydrogen atom, and R^{3b} represents any substituents described in [Table L4] to [Table L12] (hereinafter, referred to as Compound Class SX₃₀₈₂).

A compound (L-10) wherein n is 2, T represents a group represented by T39, R¹ represents S(O)CF₃, R^{3c} represents a hydrogen atom, and R^{3b} represents any substituents described in [Table L4] to [Table L12] (hereinafter, referred to as Compound Class SX₃₀₈₃).

A compound (L-10) wherein n is 2, T represents a group represented by T39, R¹ represents S(O)₂CF₃, R^{3c} represents a hydrogen atom, and R^{3b} represents any substituents described in [Table L4] to [Table L12] (hereinafter, referred to as Compound Class SX₃₀₈₄).

A compound represented by formula (L-11):

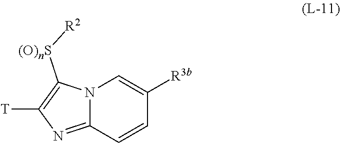

(hereinafter, referred to as Compound (L-11)),
wherein T represents a group represented by T37, R¹ represents CF₃, n is 0, R² represents a methyl group, and R^{3b} represents a hydrogen atom, a chlorine atom, CF₃, or any substituents described in [Table L4] to [Table L12](hereinafter, referred to as Compound Class SX₃₀₈₅).

A compound (L-11) wherein T represents a group represented by T37, R¹ represents CF₃, n is 1, R² represents a methyl group, and R^{3b} represents a hydrogen atom, a chlorine atom, CF₃, or any substituents described in [Table L4] to [Table L12] (hereinafter, referred to as Compound Class SX₃₀₈₆).

A compound (L-11) wherein T represents a group represented by T37, R¹ represents CF₃, n is 2, R² represents a methyl group, and R^{3b} represents a hydrogen atom, a chlorine atom, CF₃, or any substituents described in [Table L4] to [Table L12] (hereinafter, referred to as Compound Class SX₃₀₈₇).

A compound (L-11) wherein T represents a group represented by T38, R¹ represents CF₃, n is 0, R² represents a methyl group, and R^{3b} represents a hydrogen atom, a chlorine atom, CF₃, or any substituents described in [Table L4] to [Table L12] (hereinafter, referred to as Compound Class SX₃₀₈₈).

A compound (L-11) wherein T represents a group represented by T38, R¹ represents CF₃, n is 1, R² represents a methyl group, and R^{3b} represents a hydrogen atom, a chlorine atom, CF₃, or any substituents described in [Table L4] to [Table L12] (hereinafter, referred to as Compound Class SX₃₀₈₉).

A compound (L-11) wherein T represents a group represented by T38, R¹ represents CF₃, n is 2, R² represents a methyl group, and R^{3b} represents a hydrogen atom, a chlorine atom, CF₃, or any substituents described in [Table L4] to [Table L12] (hereinafter, referred to as Compound Class SX₃₀₉₀).

A compound (L-11) wherein T represents a group represented by T39, R¹ represents CF₃, n is 0, R² represents a methyl group, and R^{3b} represents a hydrogen atom, a chlorine atom, CF₃, or any substituents described in [Table L4] to [Table L12] (hereinafter, referred to as Compound Class SX₃₀₉₁).

A compound (L-11) wherein T represents a group represented by T39, R¹ represents CF₃, n is 1, R² represents a methyl group, and R^{3b} represents a hydrogen atom, a chlorine atom, CF₃, or any substituents described in [Table L4] to [Table L12] (hereinafter, referred to as Compound Class SX₃₀₉₂).

A compound (L-11) wherein T represents a group represented by T39, R¹ represents CF₃, n is 2, R² represents a methyl group, and R^{3b} represents a hydrogen atom, a chlorine atom, CF₃, or any substituents described in [Table L4] to [Table L12] (hereinafter, referred to as Compound Class SX₃₀₉₃).

A compound (L-11) wherein T represents a group represented by T37, R¹ represents C₂F5, n is 0, R² represents a methyl group, and R^{3b} represents a hydrogen atom, a chlorine atom, CF₃, or any substituents described in [Table L4] to [Table L12] (hereinafter, referred to as Compound Class SX₃₀₉₄).

A compound (L-11) wherein T represents a group represented by T37, R¹ represents C₂F5, n is 1, R² represents a methyl group, and R^{3b} represents a hydrogen atom, a chlorine atom, CF₃, or any substituents described in [Table L4] to [Table L12] (hereinafter, referred to as Compound Class SX₃₀₉₅).

A compound (L-11) wherein T represents a group represented by T37, R¹ represents C₂F5, n is 2, R² represents a methyl group, and R^{3b} represents a hydrogen atom, a chlorine atom, CF₃, or any substituents described in [Table L4] to [Table L12] (hereinafter, referred to as Compound Class SX₃₀₉₆).

A compound (L-11) wherein T represents a group represented by T38, R¹ represents C₂F5, n is 0, R² represents a methyl group, and R^{3b} represents a hydrogen atom, a chlorine atom, CF₃, or any substituents described in [Table L4] to [Table L12] (hereinafter, referred to as Compound Class SX₃₀₉₇).

A compound (L-11) wherein T represents a group represented by T38, R¹ represents C₂F5, n is 1, R² represents a methyl group, and R^{3b} represents a hydrogen atom, a chlorine atom, CF₃, or any substituents described in [Table L4] to [Table L12] (hereinafter, referred to as Compound Class SX₃₀₉₈).

A compound (L-11) wherein T represents a group represented by T38, R¹ represents C₂F5, n is 2, R² represents a methyl group, and R^{3b} represents a hydrogen atom, a chlorine atom, CF₃, or any substituents described in [Table L4] to [Table L12] (hereinafter, referred to as Compound Class SX₃₀₉₉).

A compound (L-11) wherein T represents a group represented by T39, R¹ represents C₂F5, n is 0, R² represents a methyl group, and R^{3b} represents a hydrogen atom, a chlorine atom, CF₃, or any substituents described in [Table L4] to [Table L12] (hereinafter, referred to as Compound Class SX₃₁₀₀).

A compound (L-11) wherein T represents a group represented by T39, R¹ represents C₂F5, n is 1, R² represents a methyl group, and R^{3b} represents a hydrogen atom, a chlorine atom, CF₃, or any substituents described in [Table L4] to [Table L12] (hereinafter, referred to as Compound Class SX₃₁₀₁).

A compound (L-11) wherein T represents a group represented by T39, R¹ represents C₂F5, n is 2, R² represents a methyl group, and R^{3b} represents a hydrogen atom, a chlorine atom, CF₃, or any substituents described in [Table L4] to [Table L12] (hereinafter, referred to as Compound Class SX₃₁₀₂).

A compound (L-11) wherein T represents a group represented by T37, R¹ represents SCF₃, n is 0, R² represents a methyl group, and R^{3b} represents a hydrogen atom, a chlorine atom, CF₃, or any substituents described in [Table L4] to [Table L12] (hereinafter, referred to as Compound Class SX₃₁₀₃).

A compound (L-11) wherein T represents a group represented by T37, R¹ represents SCF₃, n is 1, R² represents a methyl group, and R^{3b} represents a hydrogen atom, a chlorine atom, CF₃, or any substituents described in [Table L4] to [Table L12] (hereinafter, referred to as Compound Class SX₃₁₀₄).

A compound (L-11) wherein T represents a group represented by T37, R¹ represents SCF₃, n is 2, R² represents a methyl group, and R^{3b} represents a hydrogen atom, a chlorine atom, CF₃, or any substituents described in [Table L4] to [Table L12] (hereinafter, referred to as Compound Class SX₃₁₀₅).

A compound (L-11) wherein T represents a group represented by T38, R¹ represents SCF₃, n is 0, R² represents a methyl group, and R^{3b} represents a hydrogen atom, a chlorine atom, CF₃, or any substituents described in [Table L4] to [Table L12] (hereinafter, referred to as Compound Class SX₃₁₀₆).

A compound (L-11) wherein T represents a group represented by T38, R¹ represents SCF₃, n is 1, R² represents a methyl group, and R^{3b} represents a hydrogen atom, a chlorine atom, CF₃, or any substituents described in [Table L4] to [Table L12] (hereinafter, referred to as Compound Class SX₃₁₀₇).

A compound (L-11) wherein T represents a group represented by T38, R¹ represents SCF₃, n is 2, R² represents a methyl group, and R^{3b} represents a hydrogen atom, a chlorine atom, CF₃, or any substituents described in [Table L4] to [Table L12] (hereinafter, referred to as Compound Class SX₃₁₀₈).

A compound (L-11) wherein T represents a group represented by T39, R¹ represents SCF₃, n is 0, R² represents a methyl group, and R^{3b} represents a hydrogen atom, a chlorine atom, CF₃, or any substituents described in [Table L4] to [Table L12] (hereinafter, referred to as Compound Class SX₃₁₀₉).

A compound (L-11) wherein T represents a group represented by T39, R¹ represents SCF₃, n is 1, R² represents a methyl group, and R^{3b} represents a hydrogen atom, a chlorine atom, CF₃, or any substituents described in [Table L4] to [Table L12] (hereinafter, referred to as Compound Class SX₃₀₁₀).

A compound (L-11) wherein T represents a group represented by T39, R¹ represents SCF₃, n is 2, R² represents a methyl group, and R^{3b} represents a hydrogen atom, a chlorine atom, CF₃, or any substituents described in [Table L4] to [Table L12] (hereinafter, referred to as Compound Class SX₃₀₁₁).

A compound (L-11) wherein T represents a group represented by T37, R¹ represents S(O)CF₃, n is 0, R² represents a methyl group, and R^{3b} represents a hydrogen atom, a chlorine atom, CF₃, or any substituents described in [Table L4] to [Table L12] (hereinafter, referred to as Compound Class SX₃₁₁₂).

A compound (L-11) wherein T represents a group represented by T37, R¹ represents S(O)CF₃, n is 1, R² represents a methyl group, and R^{3b} represents a hydrogen atom, a chlorine atom, CF₃, or any substituents described in [Table L4] to [Table L12] (hereinafter, referred to as Compound Class SX₃₁₁₃).

A compound (L-11) wherein T represents a group represented by T37, R¹ represents S(O)CF₃, n is 2, R² represents a methyl group, and R^{3b} represents a hydrogen atom, a chlorine atom, CF₃, or any substituents described in [Table L4] to [Table L12] (hereinafter, referred to as Compound Class SX₃₁₁₄).

A compound (L-11) wherein T represents a group represented by T38, R¹ represents S(O)CF₃, n is 0, R² represents a methyl group, and R^{3b} represents a hydrogen atom, a chlorine atom, CF₃, or any substituents described in [Table L4] to [Table L12] (hereinafter, referred to as Compound Class SX₃₁₁₅).

A compound (L-11) wherein T represents a group represented by T38, R¹ represents S(O)CF₃, n is 1, R² represents a methyl group, and R^{3b} represents a hydrogen atom, a chlorine atom, CF₃, or any substituents described in [Table L4] to [Table L12] (hereinafter, referred to as Compound Class SX₃₁₁₆).

A compound (L-11) wherein T represents a group represented by T38, R¹ represents S(O)CF₃, n is 2, R² represents a methyl group, and R^{3b} represents a hydrogen atom, a chlorine atom, CF₃, or any substituents described in [Table L4] to [Table L12] (hereinafter, referred to as Compound Class SX₃₁₁₇).

A compound (L-11) wherein T represents a group represented by T39, R¹ represents S(O)CF₃, n is 0, R² represents a methyl group, and R^{3b} represents a hydrogen atom, a chlorine atom, CF₃, or any substituents described in [Table L4] to [Table L12] (hereinafter, referred to as Compound Class SX₃₁₁₈).

A compound (L-11) wherein T represents a group represented by T39, R¹ represents S(O)CF₃, n is 1, R² represents a methyl group, and R^{3b} represents a hydrogen atom, a chlorine atom, CF₃, or any substituents described in [Table L4] to [Table L12] (hereinafter, referred to as Compound Class SX₃₁₁₉).

A compound (L-11) wherein T represents a group represented by T39, R¹ represents S(O)CF₃, n is 2, R² represents a methyl group, and R^{3b} represents a hydrogen atom, a chlorine atom, CF₃, or any substituents described in [Table L4] to [Table L12] (hereinafter, referred to as Compound Class SX₃₁₂₀).

A compound (L-11) wherein T represents a group represented by T37, R¹ represents S(O)₂CF₃, n is 0, R² represents a methyl group, and R^{3b} represents a hydrogen atom, a chlorine atom, CF₃, or any substituents described in [Table L4] to [Table L12] (hereinafter, referred to as Compound Class SX₃₁₂₁).

A compound (L-11) wherein T represents a group represented by T37, R¹ represents S(O)₂CF₃, n is 1, R² represents a methyl group, and R^{3b} represents a hydrogen atom, a chlorine atom, CF₃, or any substituents described in [Table L4] to [Table L12] (hereinafter, referred to as Compound Class SX₃₁₂₂).

A compound (L-11) wherein T represents a group represented by T37, R¹ represents S(O)₂CF₃, n is 2, R² represents a methyl group, and R^{3b} represents a hydrogen atom, a chlorine atom, CF₃, or any substituents described in [Table L4] to [Table L12] (hereinafter, referred to as Compound Class SX₃₁₂₃).

A compound (L-11) wherein T represents a group represented by T38, R¹ represents S(O)₂CF₃, n is 0, R² represents a methyl group, and R^{3b} represents a hydrogen atom, a chlorine atom, CF₃, or any substituents described in [Table L4] to [Table L12] (hereinafter, referred to as Compound Class SX₃₁₂₄).

A compound (L-11) wherein T represents a group represented by T38, R¹ represents S(O)₂CF₃, n is 1, R² represents a methyl group, and R^{3b} represents a hydrogen atom, a chlorine atom, CF₃, or any substituents described in [Table L4] to [Table L12] (hereinafter, referred to as Compound Class SX₃₁₂₅).

A compound (L-11) wherein T represents a group represented by T38, R¹ represents S(O)₂CF₃, n is 2, R² represents a methyl group, and R^{3b} represents a hydrogen atom, a chlorine atom, CF₃, or any substituents described in [Table L4] to [Table L12] (hereinafter, referred to as Compound Class SX₃₁₂₆).

A compound (L-11) wherein T represents a group represented by T39, R¹ represents S(O)₂CF₃, n is 0, R² represents a methyl group, and R^{3b} represents a hydrogen atom, a chlorine atom, CF₃, or any substituents described in [Table L4] to [Table L12] (hereinafter, referred to as Compound Class SX₃₁₂₇).

A compound (L-11) wherein T represents a group represented by T39, R¹ represents S(O)₂CF₃, n is 1, R² represents a methyl group, and R^{3b} represents a hydrogen atom, a chlorine atom, CF₃, or any substituents described in [Table L4] to [Table L12] (hereinafter, referred to as Compound Class SX₃₁₂₈).

A compound (L-11) wherein T represents a group represented by T39, R¹ represents S(O)₂CF₃, n is 2, R² represents a methyl group, and R^{3b} represents a hydrogen atom, a chlorine atom, CF₃, or any substituents described in [Table L4] to [Table L12] (hereinafter, referred to as Compound Class SX₃₁₂₉).

A compound represented by formula (L-12):

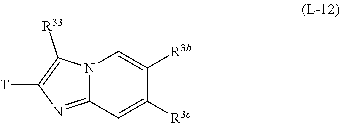

(hereinafter, referred to as Compound (L-12)),
wherein R³³ represents a hydrogen atom, T represents a group represented by T1, R^{3b} represents a hydrogen atom, R^{3c} represents a hydrogen atom, and R¹ represents any substituents described in [Table L1] to [Table L3](hereinafter, referred to as Compound Class SX₃₁₃₀).

A compound (L-12) wherein R³³ represents a hydrogen atom, T represents a group represented by T1, R^{3b} represents CF₃, R^{3c} represents a hydrogen atom, and R¹ represents any substituents described in [Table L1] to [Table L3](hereinafter, referred to as Compound Class SX₃₁₃₁).

A compound (L-12) wherein R³³ represents a hydrogen atom, T represents a group represented by T1, R^{3b} represents CF₃, R^{3c} represents a chlorine atom, and R¹ represents any substituents described in [Table L1] to [Table L3](hereinafter, referred to as Compound Class SX₃₁₃₂).

A compound (L-12) wherein R³³ represents a hydrogen atom, T represents a group represented by T1, R^{3b} represents CF₃, R^{3c} represents a bromine atom, and R¹ represents any substituents described in [Table L1] to [Table L3](hereinafter, referred to as Compound Class SX₃₁₃₃).

A compound (L-12) wherein R³³ represents a hydrogen atom, T represents a group represented by T1, R^{3b} represents CF₃, R^{3c} represents an iodine atom, and R¹ represents any substituents described in [Table L1] to [Table L3](hereinafter, referred to as Compound Class SX₃₁₃₄).

A compound (L-12) wherein R³³ represents a hydrogen atom, T represents a group represented by T1, R^{3b} represents a hydrogen atom, R^{3c} represents CF₃, and R¹ represents any substituents described in [Table L1] to [Table L3](hereinafter, referred to as Compound Class SX₃₁₃₅).

A compound (L-12) wherein R³³ represents a hydrogen atom, T represents a group represented by T1, R^{3b} represents a hydrogen atom, R^{3c} represents a chorine atom, and R¹ represents any substituents described in [Table L1] to [Table L3] (hereinafter, referred to as Compound Class SX₃₁₃₆).

A compound (L-12) wherein R³³ represents a hydrogen atom, T represents a group represented by T1, R^{3b} represents a hydrogen atom, R^{3c} represents a bromine atom, and R¹ represents any substituents described in [Table L1] to [Table L3] (hereinafter, referred to as Compound Class SX₃₁₃₇).

A compound (L-12) wherein R³³ represents a fluorine atom, T represents a group represented by T1, R^{3b} represents a hydrogen atom, R^{3c} represents a hydrogen atom, and R¹ represents any substituents described in [Table L1] to [Table L3] (hereinafter, referred to as Compound Class SX₃₁₃₈).

A compound (L-12) wherein R³³ represents a fluorine atom, T represents a group represented by T1, R^{3b} represents CF₃, R^{3c} represents a hydrogen atom, and R¹ represents any substituents described in [Table L1] to [Table L3](hereinafter, referred to as Compound Class SX₃₁₃₉).

A compound (L-12) wherein R³³ represents a fluorine atom, T represents a group represented by T1, R^{3b} represents CF₃, R^{3c} represents a chlorine atom, and R¹ represents any substituents described in [Table L1] to [Table L3](hereinafter, referred to as Compound Class SX₃₁₄₀).

A compound (L-12) wherein R³³ represents a fluorine atom, T represents a group represented by T1, R^{3b} represents CF₃, R^{3c} represents a bromine atom, and R¹ represents any substituents described in [Table L1] to [Table L3](hereinafter, referred to as Compound Class SX₃₁₄₁).

A compound (L-12) wherein R³³ represents a fluorine atom, T represents a group represented by T1, R^{3b} represents CF₃, R^{3c} represents an iodine atom, and R¹ represents any substituents described in [Table L1] to [Table L3](hereinafter, referred to as Compound Class SX₃₁₄₂).

A compound (L-12) wherein R³³ represents a fluorine atom, T represents a group represented by T1, R^{3b} represents a hydrogen atom, R^{3c} represents CF₃, and R¹ represents any substituents described in [Table L1] to [Table L3](hereinafter, referred to as Compound Class SX₃₁₄₃).

A compound (L-12) wherein R³³ represents a fluorine atom, T represents a group represented by T1, R^{3b} represents a hydrogen atom, R^{3c} represents a chorine atom, and R¹ represents any substituents described in [Table L1] to [Table L3] (hereinafter, referred to as Compound Class SX₃₁₄₄).

A compound (L-12) wherein R³³ represents a fluorine atom, T represents a group represented by T1, R^{3b} represents a hydrogen atom, R^{3c} represents a bromine atom, and R¹ represents any substituents described in [Table L1] to [Table L3] (hereinafter, referred to as Compound Class SX₃₁₄₅).

A compound (L-12) wherein R³³ represents a chlorine atom, T represents a group represented by T1, R^{3b} represents a hydrogen atom, R^{3c} represents a hydrogen atom, and R¹ represents any substituents described in [Table L1] to [Table L3] (hereinafter, referred to as Compound Class SX₃₁₄₆).

A compound (L-12) wherein R³³ represents a chlorine atom, T represents a group represented by T1, R^{3b} represents CF₃, R^{3c} represents a hydrogen atom, and R¹ represents any substituents described in [Table L1] to [Table L3](hereinafter, referred to as Compound Class SX₃₁₄₇).

A compound (L-12) wherein R³³ represents a chlorine atom, T represents a group represented by T1, R^{3b} represents CF₃, R^{3c} represents a chlorine atom, and R¹ represents any substituents described in [Table L1] to [Table L3](hereinafter, referred to as Compound Class SX₃₁₄₈).

A compound (L-12) wherein R³³ represents a chlorine atom, T represents a group represented by T1, R^{3b} represents CF₃, R^{3c} represents a bromine atom, and R¹ represents any substituents described in [Table L1] to [Table L3](hereinafter, referred to as Compound Class SX₃₁₄₉).

A compound (L-12) wherein R³³ represents a chlorine atom, T represents a group represented by T1, R^{3b} represents CF₃, R^{3c} represents an iodine atom, and R¹ represents any substituents described in [Table L1] to [Table L3](hereinafter, referred to as Compound Class SX₃₁₅₀).

A compound (L-12) wherein R³³ represents a chlorine atom, T represents a group represented by T1, R^{3b} represents a hydrogen atom, R^{3c} represents CF₃, and R¹ represents any substituents described in [Table L1] to [Table L3](hereinafter, referred to as Compound Class SX₃₁₅₁).

A compound (L-12) wherein R³³ represents a chlorine atom, T represents a group represented by T1, R^{3b} represents a hydrogen atom, R^{3c} represents a chorine atom, and R¹ represents any substituents described in [Table L1] to [Table L3] (hereinafter, referred to as Compound Class SX₃₁₅₂).

A compound (L-12) wherein R³³ represents a chlorine atom, T represents a group represented by T1, R^{3b} represents a hydrogen atom, R^{3c} represents a bromine atom, and R¹ represents any substituents described in [Table L1] to [Table L3] (hereinafter, referred to as Compound Class SX₃₁₅₃).

A compound (L-12) wherein R³³ represents a bromine atom, T represents a group represented by T1, R^{3b} represents a hydrogen atom, R^{3c} represents a hydrogen atom, and R¹ represents any substituents described in [Table L1] to [Table L3] (hereinafter, referred to as Compound Class SX₃₁₅₄).

A compound (L-12) wherein R³³ represents a bromine atom, T represents a group represented by T1, R^{3b} represents CF₃, R^{3c} represents a hydrogen atom, and R¹ represents any substituents described in [Table L1] to [Table L3](hereinafter, referred to as Compound Class SX₃₁₅₅).

A compound (L-12) wherein R³³ represents a bromine atom, T represents a group represented by T1, R^{3b} represents CF₃, R^{3c} represents a chlorine atom, and R¹ represents any substituents described in [Table L1] to [Table L3](hereinafter, referred to as Compound Class SX₃₁₅₆).

A compound (L-12) wherein R³³ represents a bromine atom, T represents a group represented by T1, R^{3b} represents CF₃, R^{3c} represents a bromine atom, and R¹ represents any substituents described in [Table L1] to [Table L3](hereinafter, referred to as Compound Class SX₃₁₅₇).

A compound (L-12) wherein R³³ represents a bromine atom, T represents a group represented by T1, R^{3b} represents CF₃, R^{3c} represents an iodine atom, and R¹ represents any substituents described in [Table L1] to [Table L3](hereinafter, referred to as Compound Class SX₃₁₅₈).

A compound (L-12) wherein R³³ represents a bromine atom, T represents a group represented by T1, R^{3b} represents a hydrogen atom, R^{3c} represents CF₃, and R¹ represents any substituents described in [Table L1] to [Table L3](hereinafter, referred to as Compound Class SX₃₁₅₉).

A compound (L-12) wherein R³³ represents a bromine atom, T represents a group represented by T1, R^{3b} represents a hydrogen atom, R^{3c} represents a chorine atom, and R¹ represents any substituents described in [Table L1] to [Table L3] (hereinafter, referred to as Compound Class SX₃₁₆₀).

A compound (L-12) wherein R³³ represents a bromine atom, T represents a group represented by T1, R^{3b} represents a hydrogen atom, R^{3c} represents a bromine atom, and R¹ represents any substituents described in [Table L1] to [Table L3] (hereinafter, referred to as Compound Class SX₃₁₆₁).

A compound (L-12) wherein R³³ represents an iodine atom, T represents a group represented by T1, R^{3b} represents a hydrogen atom, R^{3c} represents a hydrogen atom, and R¹ represents any substituents described in [Table L1] to [Table L3] (hereinafter, referred to as Compound Class SX₃₁₆₂).

A compound (L-12) wherein R³³ represents an iodine atom, T represents a group represented by T1, R^{3b} represents CF₃, R^{3c} represents a hydrogen atom, and R¹ represents any substituents described in [Table L1] to [Table L3](hereinafter, referred to as Compound Class SX₃₁₆₃).

A compound (L-12) wherein R³³ represents an iodine atom, T represents a group represented by T1, R^{3b} represents CF₃, R^{3c} represents a chlorine atom, and R¹ represents any substituents described in [Table L1] to [Table L3](hereinafter, referred to as Compound Class SX₃₁₆₄).

A compound (L-12) wherein R³³ represents an iodine atom, T represents a group represented by T1, R^{3b} represents CF₃, R^{3c} represents a bromine atom, and R¹ represents any substituents described in [Table L1] to [Table L3](hereinafter, referred to as Compound Class SX₃₁₆₅).

A compound (L-12) wherein R³³ represents an iodine atom, T represents a group represented by T1, R^{3b} represents CF₃, R^{3c} represents an iodine atom, and R¹ represents any substituents described in [Table L1] to [Table L3](hereinafter, referred to as Compound Class SX₃₁₆₆).

A compound (L-12) wherein R³³ represents an iodine atom, T represents a group represented by T1, R^{3b} represents a hydrogen atom, R^{3c} represents CF₃, and R¹ represents any substituents described in [Table L1] to [Table L3](hereinafter, referred to as Compound Class SX₃₁₆₇).

A compound (L-12) wherein R³³ represents an iodine atom, T represents a group represented by T1, R^{3b} represents a hydrogen atom, R^{3c} represents a chorine atom, and R¹ represents any substituents described in [Table L1] to [Table L3] (hereinafter, referred to as Compound Class SX₃₁₆₈).

A compound (L-12) wherein R³³ represents an iodine atom, T represents a group represented by T1, R^{3b} represents a hydrogen atom, R^{3c} represents a bromine atom, and R¹ represents any substituents described in [Table L1] to [Table L3] (hereinafter, referred to as Compound Class SX₃₁₆₉).

A compound (L-12) wherein R³³ represents a hydrogen atom, T represents a group represented by T2, R^{3b} represents a hydrogen atom, R^{3c} represents a hydrogen atom, and R¹ represents any substituents described in [Table L1] to [Table L3] (hereinafter, referred to as Compound Class SX₃₁₇₀).

A compound (L-12) wherein R³³ represents a hydrogen atom, T represents a group represented by T2, R^{3b} represents CF₃, R^{3c} represents a hydrogen atom, and R¹ represents any substituents described in [Table L1] to [Table L3](hereinafter, referred to as Compound Class SX₃₁₇₁).

A compound (L-12) wherein R³³ represents a hydrogen atom, T represents a group represented by T2, R^{3b} represents CF₃, R^{3c} represents a chlorine atom, and R¹ represents any substituents described in [Table L1] to [Table L3](hereinafter, referred to as Compound Class SX₃₁₇₂).

A compound (L-12) wherein R³³ represents a hydrogen atom, T represents a group represented by T2, R^{3b} represents CF₃, R^{3c} represents a bromine atom, and R¹ represents any substituents described in [Table L1] to [Table L3](hereinafter, referred to as Compound Class SX₃₁₇₃).

A compound (L-12) wherein R³³ represents a hydrogen atom, T represents a group represented by T2, R^{3b} represents CF₃, R^{3c} represents an iodine atom, and R¹ represents any substituents described in [Table L1] to [Table L3](hereinafter, referred to as Compound Class SX₃₁₇₄).

A compound (L-12) wherein R³³ represents a hydrogen atom, T represents a group represented by T2, R^{3b} represents a hydrogen atom, R^{3c} represents CF₃, and R¹ represents any substituents described in [Table L1] to [Table L3](hereinafter, referred to as Compound Class SX₃₁₇₅).

A compound (L-12) wherein R³³ represents a hydrogen atom, T represents a group represented by T2, R^{3b} represents a hydrogen atom, R^{3c} represents a chorine atom, and R¹ represents any substituents described in [Table L1] to [Table L3] (hereinafter, referred to as Compound Class SX₃₁₇₆).

A compound (L-12) wherein R³³ represents a hydrogen atom, T represents a group represented by T2, R^{3b} represents a hydrogen atom, R^{3c} represents a bromine atom, and R¹ represents any substituents described in [Table L1] to [Table L3] (hereinafter, referred to as Compound Class SX₃₁₇₇).

A compound (L-12) wherein R³³ represents a fluorine atom, T represents a group represented by T2, R^{3b} represents a hydrogen atom, R^{3c} represents a hydrogen atom, and R¹ represents any substituents described in [Table L1] to [Table L3] (hereinafter, referred to as Compound Class SX₃₁₇₈).

A compound (L-12) wherein R³³ represents a fluorine atom, T represents a group represented by T2, R^{3b} represents CF₃, R^{3c} represents a hydrogen atom, and R¹ represents any substituents described in [Table L1] to [Table L3](hereinafter, referred to as Compound Class SX₃₁₇₉).

A compound (L-12) wherein R³³ represents a fluorine atom, T represents a group represented by T2, R^{3b} represents CF₃, R^{3c} represents a chlorine atom, and R¹ represents any substituents described in [Table L1] to [Table L3](hereinafter, referred to as Compound Class SX₃₁₈₀).

A compound (L-12) wherein R³³ represents a fluorine atom, T represents a group represented by T2, R^{3b} represents CF₃, R^{3c} represents a bromine atom, and R¹ represents any substituents described in [Table L1] to [Table L3](hereinafter, referred to as Compound Class SX₃₁₈₁).

A compound (L-12) wherein R³³ represents a fluorine atom, T represents a group represented by T2, R^{3b} represents CF₃, R^{3c} represents an iodine atom, and R¹ represents any substituents described in [Table L1] to [Table L3](hereinafter, referred to as Compound Class SX₃₁₈₂).

A compound (L-12) wherein R³³ represents a fluorine atom, T represents a group represented by T2, R^{3b} represents a hydrogen atom, R^{3c} represents CF₃, and R¹ represents any substituents described in [Table L1] to [Table L3](hereinafter, referred to as Compound Class SX₃₁₈₃).

A compound (L-12) wherein R³³ represents a fluorine atom, T represents a group represented by T2, R^{3b} represents a hydrogen atom, R^{3c} represents a chorine atom, and R¹ represents any substituents described in [Table L1] to [Table L3] (hereinafter, referred to as Compound Class SX₃₁₈₄).

A compound (L-12) wherein R³³ represents a fluorine atom, T represents a group represented by T2, R^{3b} represents a hydrogen atom, R^{3c} represents a bromine atom, and R¹ represents any substituents described in [Table L1] to [Table L3] (hereinafter, referred to as Compound Class SX₃₁₈₅).

A compound (L-12) wherein R³³ represents a chlorine atom, T represents a group represented by T2, R^{3b} represents a hydrogen atom, R^{3c} represents a hydrogen atom, and R¹ represents any substituents described in [Table L1] to [Table L3] (hereinafter, referred to as Compound Class SX₃₁₈₆).

A compound (L-12) wherein R³³ represents a chlorine atom, T represents a group represented by T2, R^{3b} represents CF₃, R^{3c} represents a hydrogen atom, and R¹ represents any substituents described in [Table L1] to [Table L3](hereinafter, referred to as Compound Class SX₃₁₈₇).

A compound (L-12) wherein R³³ represents a chlorine atom, T represents a group represented by T2, R^{3b} represents CF₃, R^{3c} represents a chlorine atom, and R¹ represents any substituents described in [Table L1] to [Table L3](hereinafter, referred to as Compound Class SX₃₁₈₈).

A compound (L-12) wherein R³³ represents a chlorine atom, T represents a group represented by T2, R^{3b} represents CF₃, R^{3c} represents a bromine atom, and R¹ represents any substituents described in [Table L1] to [Table L3](hereinafter, referred to as Compound Class SX₃₁₈₉).

A compound (L-12) wherein R³³ represents a chlorine atom, T represents a group represented by T2, R^{3b} represents CF₃, R^{3c} represents an iodine atom, and R¹ represents any substituents described in [Table L1] to [Table L3](hereinafter, referred to as Compound Class SX₃₁₉₀).

A compound (L-12) wherein R³³ represents a chlorine atom, T represents a group represented by T2, R^{3b} represents a hydrogen atom, R^{3c} represents CF₃, and R¹ represents any substituents described in [Table L1] to [Table L3](hereinafter, referred to as Compound Class SX₃₁₉₁).

A compound (L-12) wherein R³³ represents a chlorine atom, T represents a group represented by T2, R^{3b} represents a hydrogen atom, R^{3c} represents a chorine atom, and R¹ represents any substituents described in [Table L1] to [Table L3] (hereinafter, referred to as Compound Class SX₃₁₉₂).

A compound (L-12) wherein R³³ represents a chlorine atom, T represents a group represented by T2, R^{3b} represents a hydrogen atom, R^{3c} represents a bromine atom, and R¹ represents any substituents described in [Table L1] to [Table L3] (hereinafter, referred to as Compound Class SX₃₁₉₃).

A compound (L-12) wherein R³³ represents a bromine atom, T represents a group represented by T2, R^{3b} represents a hydrogen atom, R^{3c} represents a hydrogen atom, and R¹ represents any substituents described in [Table L1] to [Table L3] (hereinafter, referred to as Compound Class SX₃₁₉₄).

A compound (L-12) wherein R³³ represents a bromine atom, T represents a group represented by T2, R^{3b} represents CF₃, R^{3c} represents a hydrogen atom, and R¹ represents any substituents described in [Table L1] to [Table L3](hereinafter, referred to as Compound Class SX₃₁₉₅).

A compound (L-12) wherein R³³ represents a bromine atom, T represents a group represented by T2, R^{3b} represents CF₃, R^{3c} represents a chlorine atom, and R¹ represents any substituents described in [Table L1] to [Table L3](hereinafter, referred to as Compound Class SX₃₁₉₆).

A compound (L-12) wherein R³³ represents a bromine atom, T represents a group represented by T2, R^{3b} represents CF₃, R^{3c} represents a bromine atom, and R¹ represents any substituents described in [Table L1] to [Table L3](hereinafter, referred to as Compound Class SX₃₁₉₇).

A compound (L-12) wherein R³³ represents a bromine atom, T represents a group represented by T2, R^{3b} represents CF₃, R^{3c} represents an iodine atom, and R¹ represents any substituents described in [Table L1] to [Table L3](hereinafter, referred to as Compound Class SX₃₁₉₈).

A compound (L-12) wherein R³³ represents a bromine atom, T represents a group represented by T2, R^{3b} represents a hydrogen atom, R^{3c} represents CF₃, and R¹ represents any substituents described in [Table L1] to [Table L3](hereinafter, referred to as Compound Class SX₃₁₉₉).

A compound (L-12) wherein R³³ represents a bromine atom, T represents a group represented by T2, R^{3b} represents a hydrogen atom, R^{3c} represents a chorine atom, and R¹ represents any substituents described in [Table L1] to [Table L3] (hereinafter, referred to as Compound Class SX₃₂₀₀).

A compound (L-12) wherein R³³ represents a bromine atom, T represents a group represented by T2, R^{3b} represents a hydrogen atom, R^{3c} represents a bromine atom, and R¹ represents any substituents described in [Table L1] to [Table L3] (hereinafter, referred to as Compound Class SX₃₂₀₁).

A compound (L-12) wherein R³³ represents an iodine atom, T represents a group represented by T2, R^{3b} represents a hydrogen atom, R^{3c} represents a hydrogen atom, and R¹ represents any substituents described in [Table L1] to [Table L3] (hereinafter, referred to as Compound Class SX₃₂₀₂).

A compound (L-12) wherein R³³ represents an iodine atom, T represents a group represented by T2, R^{3b} represents CF₃, R^{3c} represents a hydrogen atom, and R¹ represents any substituents described in [Table L1] to [Table L3](hereinafter, referred to as Compound Class SX₃₂₀₃).

A compound (L-12) wherein R³³ represents an iodine atom, T represents a group represented by T2, R^{3b} represents CF₃, R^{3c} represents a chlorine atom, and R¹ represents any substituents described in [Table L1] to [Table L3](hereinafter, referred to as Compound Class SX₃₂₀₄).

A compound (L-12) wherein R³³ represents an iodine atom, T represents a group represented by T2, R^{3b} represents CF₃, R^{3c} represents a bromine atom, and R¹ represents any substituents described in [Table L1] to [Table L3](hereinafter, referred to as Compound Class SX₃₂₀₅).

A compound (L-12) wherein R³³ represents an iodine atom, T represents a group represented by T2, R^{3b} represents CF₃, R^{3c} represents an iodine atom, and R¹ represents any substituents described in [Table L1] to [Table L3](hereinafter, referred to as Compound Class SX₃₂₀₆).

A compound (L-12) wherein R³³ represents an iodine atom, T represents a group represented by T2, R^{3b} represents a hydrogen atom, R^{3c} represents CF₃, and R¹ represents any substituents described in [Table L1] to [Table L3](hereinafter, referred to as Compound Class SX₃₂₀₇).

A compound (L-12) wherein R³³ represents an iodine atom, T represents a group represented by T2, R^{3b} represents a hydrogen atom, R^{3c} represents a chorine atom, and R¹ represents any substituents described in [Table L1] to [Table L3] (hereinafter, referred to as Compound Class SX₃₂₀₈).

A compound (L-12) wherein R³³ represents an iodine atom, T represents a group represented by T2, R^{3b} represents a hydrogen atom, R^{3c} represents a bromine atom, and R¹ represents any substituents described in [Table L1] to [Table L3] (hereinafter, referred to as Compound Class SX₃₂₀₉).

A compound (L-12) wherein R³³ represents a hydrogen atom, T represents a group represented by T3, R^{3b} represents a hydrogen atom, R^{3c} represents a hydrogen atom, and R¹ represents any substituents described in [Table L1] to [Table L3] (hereinafter, referred to as Compound Class SX₃₂₁₀).

A compound (L-12) wherein R³³ represents a hydrogen atom, T represents a group represented by T3, R^{3b} represents CF₃, R^{3c} represents a hydrogen atom, and R¹ represents any substituents described in [Table L1] to [Table L3](hereinafter, referred to as Compound Class SX₃₂₁₁).

A compound (L-12) wherein R³³ represents a hydrogen atom, T represents a group represented by T3, R^{3b} represents CF₃, R^{3c} represents a chlorine atom, and R¹ represents any substituents described in [Table L1] to [Table L3](hereinafter, referred to as Compound Class SX₃₂₁₂).

A compound (L-12) wherein R³³ represents a hydrogen atom, T represents a group represented by T3, R^{3b} represents CF₃, R^{3c} represents a bromine atom, and R¹ represents any substituents described in [Table L1] to [Table L3](hereinafter, referred to as Compound Class SX₃₂₁₃).

A compound (L-12) wherein R³³ represents a hydrogen atom, T represents a group represented by T3, R^{3b} represents CF₃, R^{3c} represents an iodine atom, and R¹ represents any substituents described in [Table L1] to [Table L3](hereinafter, referred to as Compound Class SX₃₂₁₄).

A compound (L-12) wherein R³³ represents a hydrogen atom, T represents a group represented by T3, R^{3b} represents a hydrogen atom, R^{3c} represents CF₃, and R¹ represents any substituents described in [Table L1] to [Table L3](hereinafter, referred to as Compound Class SX₃₂₁₅).

A compound (L-12) wherein R³³ represents a hydrogen atom, T represents a group represented by T3, R^{3b} represents a hydrogen atom, R^{3c} represents a chorine atom, and R¹ represents any substituents described in [Table L1] to [Table L3] (hereinafter, referred to as Compound Class SX₃₂₁₆).

A compound (L-12) wherein R³³ represents a hydrogen atom, T represents a group represented by T3, R^{3b} represents a hydrogen atom, R^{3c} represents a bromine atom, and R¹ represents any substituents described in [Table L1] to [Table L3] (hereinafter, referred to as Compound Class SX₃₂₁₇).

A compound (L-12) wherein R³³ represents a fluorine atom, T represents a group represented by T3, R^{3b} represents a hydrogen atom, R^{3c} represents a hydrogen atom, and R¹ represents any substituents described in [Table L1] to [Table L3] (hereinafter, referred to as Compound Class SX₃₂₁₈).

A compound (L-12) wherein R³³ represents a fluorine atom, T represents a group represented by T3, R^{3b} represents CF₃, R^{3c} represents a hydrogen atom, and R¹ represents any substituents described in [Table L1] to [Table L3](hereinafter, referred to as Compound Class SX₃₂₁₉).

A compound (L-12) wherein R³³ represents a fluorine atom, T represents a group represented by T3, R^{3b} represents CF₃, R^{3c} represents a chlorine atom, and R¹ represents any substituents described in [Table L1] to [Table L3](hereinafter, referred to as Compound Class SX₃₂₂₀).

A compound (L-12) wherein R³³ represents a fluorine atom, T represents a group represented by T3, R^{3b} represents CF₃, R^{3c} represents a bromine atom, and R¹ represents any substituents described in [Table L1] to [Table L3](hereinafter, referred to as Compound Class SX₃₂₂₁).

A compound (L-12) wherein R³³ represents a fluorine atom, T represents a group represented by T3, R^{3b} represents CF₃, R^{3c} represents an iodine atom, and R¹ represents any substituents described in [Table L1] to [Table L3](hereinafter, referred to as Compound Class SX₃₂₂₂).

A compound (L-12) wherein R³³ represents a fluorine atom, T represents a group represented by T3, R^{3b} represents a hydrogen atom, R^{3c} represents CF₃, and R¹ represents any substituents described in [Table L1] to [Table L3](hereinafter, referred to as Compound Class SX₃₂₂₃).

A compound (L-12) wherein R³³ represents a fluorine atom, T represents a group represented by T3, R^{3b} represents a hydrogen atom, R^{3c} represents a chorine atom, and R¹ represents any substituents described in [Table L1] to [Table L3] (hereinafter, referred to as Compound Class SX₃₂₂₄).

A compound (L-12) wherein R³³ represents a fluorine atom, T represents a group represented by T3, R^{3b} represents a hydrogen atom, R^{3c} represents a bromine atom, and R¹ represents any substituents described in [Table L1] to [Table L3] (hereinafter, referred to as Compound Class SX₃₂₂₅).

A compound (L-12) wherein R³³ represents a chlorine atom, T represents a group represented by T3, R^{3b} represents a hydrogen atom, R^{3c} represents a hydrogen atom, and R¹ represents any substituents described in [Table L1] to [Table L3] (hereinafter, referred to as Compound Class SX₃₂₂₆).

A compound (L-12) wherein R³³ represents a chlorine atom, T represents a group represented by T3, R^{3b} represents CF₃, R^{3c} represents a hydrogen atom, and R¹ represents any substituents described in [Table L1] to [Table L3](hereinafter, referred to as Compound Class SX₃₂₂₇).

A compound (L-12) wherein R³³ represents a chlorine atom, T represents a group represented by T3, R^{3b} represents CF₃, R^{3c} represents a chlorine atom, and R¹ represents any substituents described in [Table L1] to [Table L3](hereinafter, referred to as Compound Class SX₃₂₂₈).

A compound (L-12) wherein R³³ represents a chlorine atom, T represents a group represented by T3, R^{3b} represents CF₃, R^{3c} represents a bromine atom, and R¹ represents any substituents described in [Table L1] to [Table L3](hereinafter, referred to as Compound Class SX₃₂₂₉).

A compound (L-12) wherein R³³ represents a chlorine atom, T represents a group represented by T3, R^{3b} represents CF₃, R^{3c} represents an iodine atom, and R¹ represents any substituents described in [Table L1] to [Table L3](hereinafter, referred to as Compound Class SX₃₂₃₀).

A compound (L-12) wherein R³³ represents a chlorine atom, T represents a group represented by T3, R^{3b} represents a hydrogen atom, R^{3c} represents CF₃, and R¹ represents any substituents described in [Table L1] to [Table L3](hereinafter, referred to as Compound Class SX₃₂₃₁).

A compound (L-12) wherein R³³ represents a chlorine atom, T represents a group represented by T3, R^{3b} represents a hydrogen atom, R^{3c} represents a chorine atom, and R¹ represents any substituents described in [Table L1] to [Table L3] (hereinafter, referred to as Compound Class SX₃₂₃₂).

A compound (L-12) wherein R³³ represents a chlorine atom, T represents a group represented by T3, R^{3b} represents a hydrogen atom, R^{3c} represents a bromine atom, and R¹ represents any substituents described in [Table L1] to [Table L3] (hereinafter, referred to as Compound Class SX₃₂₃₃).

A compound (L-12) wherein R³³ represents a bromine atom, T represents a group represented by T3, R^{3b} represents a hydrogen atom, R^{3c} represents a hydrogen atom, and R¹ represents any substituents described in [Table L1] to [Table L3] (hereinafter, referred to as Compound Class SX₃₂₃₄).

A compound (L-12) wherein R³³ represents a bromine atom, T represents a group represented by T3, R^{3b} represents CF₃, R^{3c} represents a hydrogen atom, and R¹ represents any substituents described in [Table L1] to [Table L3](hereinafter, referred to as Compound Class SX₃₂₃₅).

A compound (L-12) wherein R³³ represents a bromine atom, T represents a group represented by T3, R^{3b} represents CF₃, R^{3c} represents a chlorine atom, and R¹ represents any substituents described in [Table L1] to [Table L3](hereinafter, referred to as Compound Class SX₃₂₃₆).

A compound (L-12) wherein R³³ represents a bromine atom, T represents a group represented by T3, R^{3b} represents CF₃, R^{3c} represents a bromine atom, and R¹ represents any substituents described in [Table L1] to [Table L3](hereinafter, referred to as Compound Class SX₃₂₃₇).

A compound (L-12) wherein R³³ represents a bromine atom, T represents a group represented by T3, R^{3b} represents CF₃, R^{3c} represents an iodine atom, and R¹ represents any substituents described in [Table L1] to [Table L3](hereinafter, referred to as Compound Class SX₃₂₃₈).

A compound (L-12) wherein R³³ represents a bromine atom, T represents a group represented by T3, R^{3b} represents a hydrogen atom, R^{3c} represents CF₃, and R¹ represents any substituents described in [Table L1] to [Table L3](hereinafter, referred to as Compound Class SX₃₂₃₉).

A compound (L-12) wherein R³³ represents a bromine atom, T represents a group represented by T3, R^{3b} represents a hydrogen atom, R^{3c} represents a chorine atom, and R¹ represents any substituents described in [Table L1] to [Table L3] (hereinafter, referred to as Compound Class SX₃₂₄₀).

A compound (L-12) wherein R³³ represents a bromine atom, T represents a group represented by T3, R^{3b} represents a hydrogen atom, R^{3c} represents a bromine atom, and R¹ represents any substituents described in [Table L1] to [Table L3] (hereinafter, referred to as Compound Class SX₃₂₄₁).

A compound (L-12) wherein R³³ represents an iodine atom, T represents a group represented by T3, R^{3b} represents a hydrogen atom, R^{3c} represents a hydrogen atom, and R¹ represents any substituents described in [Table L1] to [Table L3] (hereinafter, referred to as Compound Class SX₃₂₄₂).

A compound (L-12) wherein R³³ represents an iodine atom, T represents a group represented by T3, R^{3b} represents CF₃, R^{3c} represents a hydrogen atom, and R¹ represents any substituents described in [Table L1] to [Table L3](hereinafter, referred to as Compound Class SX₃₂₄₃).

A compound (L-12) wherein R³³ represents an iodine atom, T represents a group represented by T3, R^{3b} represents CF₃, R^{3c} represents a chlorine atom, and R¹ represents any substituents described in [Table L1] to [Table L3](hereinafter, referred to as Compound Class SX₃₂₄₄).

A compound (L-12) wherein R³³ represents an iodine atom, T represents a group represented by T3, R^{3b} represents CF₃, R^{3c} represents a bromine atom, and R¹ represents any substituents described in [Table L1] to [Table L3](hereinafter, referred to as Compound Class SX₃₂₄₅).

A compound (L-12) wherein R³³ represents an iodine atom, T represents a group represented by T3, R^{3b} represents CF₃, R^{3c} represents an iodine atom, and R¹ represents any substituents described in [Table L1] to [Table L3](hereinafter, referred to as Compound Class SX₃₂₄₆).

A compound (L-12) wherein R³³ represents an iodine atom, T represents a group represented by T3, R^{3b} represents a hydrogen atom, R^{3c} represents CF₃, and R¹ represents any substituents described in [Table L1] to [Table L3](hereinafter, referred to as Compound Class SX₃₂₄₇).

A compound (L-12) wherein R³³ represents an iodine atom, T represents a group represented by T3, R^{3b} represents a hydrogen atom, R^{3c} represents a chorine atom, and R¹ represents any substituents described in [Table L1] to [Table L3] (hereinafter, referred to as Compound Class SX₃₂₄₈).

A compound (L-12) wherein R³³ represents an iodine atom, T represents a group represented by T3, R^{3b} represents a hydrogen atom, R^{3c} represents a bromine atom, and R¹ represents any substituents described in [Table L1] to [Table L3] (hereinafter, referred to as Compound Class SX₃₂₄₉).

A compound represented by formula (L-13):

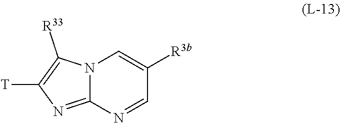

(hereinafter, referred to as Compound (L-13))
wherein R³³ represents a hydrogen atom, T represents a group represented by T1, R^{3b} represents a hydrogen atom, R^{3c} represents a hydrogen atom, and R¹ represents any substituents described in [Table L1] to [Table L3](hereinafter, referred to as Compound Class SX₃₂₅₀).

A compound (L-13) wherein R³³ represents a hydrogen atom, T represents a group represented by T1, R^{3b} represents CF₃, R^{3c} represents a hydrogen atom, and R¹ represents any substituents described in [Table L1] to [Table L3](hereinafter, referred to as Compound Class SX₃₂₅₁).

A compound (L-13) wherein R³³ represents a hydrogen atom, T represents a group represented by T1, R^{3b} represents a chlorine atom, R^{3c} represents a hydrogen atom, and R¹ represents any substituents described in [Table L1] to [Table L3] (hereinafter, referred to as Compound Class SX₃₂₅₂).

A compound (L-13) wherein R³³ represents a hydrogen atom, T represents a group represented by T1, R^{3b} represents a bromine atom, R^{3c} represents a hydrogen atom, and R¹ represents any substituents described in [Table L1] to [Table L3] (hereinafter, referred to as Compound Class SX₃₂₅₃).

A compound (L-13) wherein R³³ represents a hydrogen atom, T represents a group represented by T1, R^{3b} represents an iodine atom, R^{3c} represents a hydrogen atom, and R¹ represents any substituents described in [Table L1] to [Table L3](hereinafter, referred to as Compound Class SX₃₂₅₄).

A compound (L-13) wherein R³³ represents a hydrogen atom, T represents a group represented by T1, R^{3b} represents a hydrogen atom, R^{3c} represents CF₃, and R¹ represents any substituents described in [Table L1] to [Table L3](hereinafter, referred to as Compound Class SX₃₂₅₅).

A compound (L-13) wherein R³³ represents a hydrogen atom, T represents a group represented by T1, R^{3b} represents a hydrogen atom, R^{3c} represents a chlorine atom, and R¹ represents any substituents described in [Table L1] to [Table L3] (hereinafter, referred to as Compound Class SX₃₂₅₆).

A compound (L-13) wherein R³³ represents a hydrogen atom, T represents a group represented by T1, R^{3b} represents a hydrogen atom, R^{3c} represents a bromine atom, and R¹ represents any substituents described in [Table L1] to [Table L3] (hereinafter, referred to as Compound Class SX₃₂₅₇).

A compound (L-13) wherein R³³ represents a fluorine atom, T represents a group represented by T1, R^{3b} represents a hydrogen atom, R^{3c} represents a hydrogen atom, and R¹ represents any substituents described in [Table L1] to [Table L3] (hereinafter, referred to as Compound Class SX₃₂₅₈).

A compound (L-13) wherein R³³ represents a fluorine atom, T represents a group represented by T1, R^{3b} represents CF₃, R^{3c} represents a hydrogen atom, and R¹ represents any substituents described in [Table L1] to [Table L3](hereinafter, referred to as Compound Class SX₃₂₅₉).

A compound (L-13) wherein R³³ represents a fluorine atom, T represents a group represented by T1, R^{3b} represents a chlorine atom, R^{3c} represents a hydrogen atom, and R¹ represents any substituents described in [Table L1] to [Table L3] (hereinafter, referred to as Compound Class SX₃₂₆).

A compound (L-13) wherein R³³ represents a fluorine atom, T represents a group represented by T1, R^{3b} represents a bromine atom, R^{3c} represents a hydrogen atom, and R¹ represents any substituents described in [Table L1] to [Table L3] (hereinafter, referred to as Compound Class SX₃₂₆₁).

A compound (L-13) wherein R³³ represents a fluorine atom, T represents a group represented by T1, R^{3b} represents an iodine atom, R^{3c} represents a hydrogen atom, and R¹ represents any substituents described in [Table L1] to [Table L3](hereinafter, referred to as Compound Class SX₃₂₆₂).

A compound (L-13) wherein R³³ represents a fluorine atom, T represents a group represented by T1, R^{3b} represents a hydrogen atom, R^{3c} represents CF₃, and R¹ represents any substituents described in [Table L1] to [Table L3](hereinafter, referred to as Compound Class SX₃₂₆₃).

A compound (L-13) wherein R³³ represents a fluorine atom, T represents a group represented by T1, R^{3b} represents a hydrogen atom, R^{3c} represents a chlorine atom, and R¹ represents any substituents described in [Table L1] to [Table L3] (hereinafter, referred to as Compound Class SX₃₂₆₄).

A compound (L-13) wherein R³³ represents a fluorine atom, T represents a group represented by T1, R^{3b} represents a hydrogen atom, R^{3c} represents a bromine atom, and R¹ represents any substituents described in [Table L1] to [Table L3] (hereinafter, referred to as Compound Class SX₃₂₆₅).

A compound (L-13) wherein R³³ represents a chlorine atom, T represents a group represented by T1, R^{3b} represents a hydrogen atom, R^{3c} represents a hydrogen atom, and R¹ represents any substituents described in [Table L1] to [Table L3] (hereinafter, referred to as Compound Class SX₃₂₆₆).

A compound (L-13) wherein R³³ represents a chlorine atom, T represents a group represented by T1, R^{3b} represents CF₃, R^{3c} represents a hydrogen atom, and R¹ represents any substituents described in [Table L1] to [Table L3](hereinafter, referred to as Compound Class SX₃₂₆₇).

A compound (L-13) wherein R³³ represents a chlorine atom, T represents a group represented by T1, R^{3b} represents a chlorine atom, R^{3c} represents a hydrogen atom, and R¹ represents any substituents described in [Table L1] to [Table L3] (hereinafter, referred to as Compound Class SX₃₂₆₈).

A compound (L-13) wherein R³³ represents a chlorine atom, T represents a group represented by T1, R^{3b} represents a bromine atom, R^{3c} represents a hydrogen atom, and R¹ represents any substituents described in [Table L1] to [Table L3] (hereinafter, referred to as Compound Class SX₃₂₆₉).

A compound (L-13) wherein R³³ represents a chlorine atom, T represents a group represented by T1, R^{3b} represents an iodine atom, R^{3c} represents a hydrogen atom, and R¹ represents any substituents described in [Table L1] to [Table L3](hereinafter, referred to as Compound Class SX₃₂₇₀).

A compound (L-13) wherein R³³ represents a chlorine atom, T represents a group represented by T1, R^{3b} represents a hydrogen atom, R^{3c} represents CF₃, and R¹ represents any substituents described in [Table L1] to [Table L3](hereinafter, referred to as Compound Class SX₃₂₇₁).

A compound (L-13) wherein R³³ represents a chlorine atom, T represents a group represented by T1, R^{3b} represents a hydrogen atom, R^{3c} represents a chlorine atom, and R¹ represents any substituents described in [Table L1] to [Table L3] (hereinafter, referred to as Compound Class SX₃₂₇₂).

A compound (L-13) wherein R³³ represents a chlorine atom, T represents a group represented by T1, R^{3b} represents a hydrogen atom, R^{3c} represents a bromine atom, and R¹ represents any substituents described in [Table L1] to [Table L3] (hereinafter, referred to as Compound Class SX₃₂₇₃).

A compound (L-13) wherein R³³ represents a bromine atom, T represents a group represented by T1, R^{3b} represents a hydrogen atom, R^{3c} represents a hydrogen atom, and R¹ represents any substituents described in [Table L1] to [Table L3] (hereinafter, referred to as Compound Class SX₃₂₇₄).

A compound (L-13) wherein R³³ represents a bromine atom, T represents a group represented by T1, R^{3b} represents CF₃, R^{3c} represents a hydrogen atom, and R¹ represents any substituents described in [Table L1] to [Table L3](hereinafter, referred to as Compound Class SX₃₂₇₅).

A compound (L-13) wherein R³³ represents a bromine atom, T represents a group represented by T1, R^{3b} represents a chlorine atom, R^{3c} represents a hydrogen atom, and R¹ represents any substituents described in [Table L1] to [Table L3] (hereinafter, referred to as Compound Class SX₃₂₇₆).

A compound (L-13) wherein R³³ represents a bromine atom, T represents a group represented by T1, R^{3b} represents a bromine atom, R^{3c} represents a hydrogen atom, and R¹ represents any substituents described in [Table L1] to [Table L3] (hereinafter, referred to as Compound Class SX₃₂₇₇).

A compound (L-13) wherein R³³ represents a bromine atom, T represents a group represented by T1, R^{3b} represents an iodine atom, R^{3c} represents a hydrogen atom, and R¹ represents any substituents described in [Table L1] to [Table L3](hereinafter, referred to as Compound Class SX₃₂₇₈).

A compound (L-13) wherein R³³ represents a bromine atom, T represents a group represented by T1, R^{3b} represents a hydrogen atom, R^{3c} represents CF₃, and R¹ represents any substituents described in [Table L1] to [Table L3](hereinafter, referred to as Compound Class SX₃₂₇₉).

A compound (L-13) wherein R³³ represents a bromine atom, T represents a group represented by T1, R^{3b} represents a hydrogen atom, R^{3c} represents a chlorine atom, and R¹ represents any substituents described in [Table L1] to [Table L3] (hereinafter, referred to as Compound Class SX₃₂₀).

A compound (L-13) wherein R³³ represents a bromine atom, T represents a group represented by T1, R^{3b} represents a hydrogen atom, R^{3c} represents a bromine atom, and R¹ represents any substituents described in [Table L1] to [Table L3] (hereinafter, referred to as Compound Class SX₃₂₈₁).

A compound (L-13) wherein R³³ represents an iodine atom, T represents a group represented by T1, R^{3b} represents a hydrogen atom, R^{3c} represents a hydrogen atom, and R¹ represents any substituents described in [Table L1] to [Table L3] (hereinafter, referred to as Compound Class SX₃₂₈₂).

A compound (L-13) wherein R³³ represents an iodine atom, T represents a group represented by T1, R^{3b} represents CF₃, R^{3c} represents a hydrogen atom, and R¹ represents any substituents described in [Table L1] to [Table L3](hereinafter, referred to as Compound Class SX₃₂₈₃).

A compound (L-13) wherein R³³ represents an iodine atom, T represents a group represented by T1, R^{3b} represents a chlorine atom, R^{3c} represents a hydrogen atom, and R¹ represents any substituents described in [Table L1] to [Table L3] (hereinafter, referred to as Compound Class SX₃₂₈₄).

A compound (L-13) wherein R³³ represents an iodine atom, T represents a group represented by T1, R^{3b} represents a bromine atom, R^{3c} represents a hydrogen atom, and R¹ represents any substituents described in [Table L1] to [Table L3] (hereinafter, referred to as Compound Class SX₃₂₈₅).

A compound (L-13) wherein R³³ represents an iodine atom, T represents a group represented by T1, R^{3b} represents an iodine atom, R^{3c} represents a hydrogen atom, and R¹ represents any substituents described in [Table L1] to [Table L3](hereinafter, referred to as Compound Class SX₃₂₆).

A compound (L-13) wherein R³³ represents an iodine atom, T represents a group represented by T1, R^{3b} represents a hydrogen atom, R^{3c} represents CF₃, and R¹ represents any substituents described in [Table L1] to [Table L3](hereinafter, referred to as Compound Class SX₃₂₈₇).

A compound (L-13) wherein R³³ represents an iodine atom, T represents a group represented by T1, R^{3b} represents a hydrogen atom, R^{3c} represents a chlorine atom, and R¹ represents any substituents described in [Table L1] to [Table L3] (hereinafter, referred to as Compound Class SX₃₂₈₈).

A compound (L-13) wherein R³³ represents an iodine atom, T represents a group represented by T1, R^{3b} represents a hydrogen atom, R^{3c} represents a bromine atom, and R¹ represents any substituents described in [Table L1] to [Table L3] (hereinafter, referred to as Compound Class SX₃₂₈₉).

A compound (L-13) wherein R³³ represents a hydrogen atom, T represents a group represented by T2, R^{3b} represents a hydrogen atom, R^{3c} represents a hydrogen atom, and R¹ represents any substituents described in [Table L1] to [Table L3] (hereinafter, referred to as Compound Class SX₃₂₉).

A compound (L-13) wherein R³³ represents a hydrogen atom, T represents a group represented by T2, R^{3b} represents CF₃, R^{3c} represents a hydrogen atom, and R¹ represents any substituents described in [Table L1] to [Table L3](hereinafter, referred to as Compound Class SX₃₂₉₁).

A compound (L-13) wherein R³³ represents a hydrogen atom, T represents a group represented by T2, R^{3b} represents a chlorine atom, R^{3c} represents a hydrogen atom, and R¹ represents any substituents described in [Table L1] to [Table L3] (hereinafter, referred to as Compound Class SX₃₂₉₂).

A compound (L-13) wherein R³³ represents a hydrogen atom, T represents a group represented by T2, R^{3b} represents a bromine atom, R^{3c} represents a hydrogen atom, and R¹ represents any substituents described in [Table L1] to [Table L3] (hereinafter, referred to as Compound Class SX₃₂₉₃).

A compound (L-13) wherein R³³ represents a hydrogen atom, T represents a group represented by T2, R^{3b} represents an iodine atom, R^{3c} represents a hydrogen atom, and R¹ represents any substituents described in [Table L1] to [Table L3](hereinafter, referred to as Compound Class SX₃₂₉₄).

A compound (L-13) wherein R³³ represents a hydrogen atom, T represents a group represented by T2, R^{3b} represents a hydrogen atom, R^{3c} represents CF₃, and R¹ represents any substituents described in [Table L1] to [Table L3](hereinafter, referred to as Compound Class SX₃₂₉₅).

A compound (L-13) wherein R³³ represents a hydrogen atom, T represents a group represented by T2, R^{3b} represents a hydrogen atom, R^{3c} represents a chlorine atom, and R¹ represents any substituents described in [Table L1] to [Table L3] (hereinafter, referred to as Compound Class SX₃₂₉₆).

A compound (L-13) wherein R³³ represents a hydrogen atom, T represents a group represented by T2, R^{3b} represents a hydrogen atom, R^{3c} represents a bromine atom, and R¹ represents any substituents described in [Table L1] to [Table L3] (hereinafter, referred to as Compound Class SX₃₂₉₇).

A compound (L-13) wherein R³³ represents a fluorine atom, T represents a group represented by T2, R^{3b} represents a hydrogen atom, R^{3c} represents a hydrogen atom, and R¹ represents any substituents described in [Table L1] to [Table L3] (hereinafter, referred to as Compound Class SX₃₂₉₈).

A compound (L-13) wherein R³³ represents a fluorine atom, T represents a group represented by T2, R^{3b} represents CF₃, R^{3c} represents a hydrogen atom, and R¹ represents any substituents described in [Table L1] to [Table L3](hereinafter, referred to as Compound Class SX₃₂₉₉).

A compound (L-13) wherein R³³ represents a fluorine atom, T represents a group represented by T2, R^{3b} represents a chlorine atom, R^{3c} represents a hydrogen atom, and R¹ represents any substituents described in [Table L1] to [Table L3] (hereinafter, referred to as Compound Class SX₃₃₀₀).

A compound (L-13) wherein R³³ represents a fluorine atom, T represents a group represented by T2, R^{3b} represents a bromine atom, R^{3c} represents a hydrogen atom, and R¹ represents any substituents described in [Table L1] to [Table L3] (hereinafter, referred to as Compound Class SX₃₃₀₁).

A compound (L-13) wherein R³³ represents a fluorine atom, T represents a group represented by T2, R^{3b} represents an iodine atom, R^{3c} represents a hydrogen atom, and R¹ represents any substituents described in [Table L1] to [Table L3](hereinafter, referred to as Compound Class SX₃₃₀₂).

A compound (L-13) wherein R³³ represents a fluorine atom, T represents a group represented by T2, R^{3b} represents a hydrogen atom, R^{3c} represents CF₃, and R¹ represents any substituents described in [Table L1] to [Table L3](hereinafter, referred to as Compound Class SX₃₃₀₃).

A compound (L-13) wherein R³³ represents a fluorine atom, T represents a group represented by T2, R^{3b} represents a hydrogen atom, R^{3c} represents a chlorine atom, and R¹ represents any substituents described in [Table L1] to [Table L3] (hereinafter, referred to as Compound Class SX₃₃₀₄).

A compound (L-13) wherein R³³ represents a fluorine atom, T represents a group represented by T2, R^{3b} represents a hydrogen atom, R^{3c} represents a bromine atom, and R¹ represents any substituents described in [Table L1] to [Table L3] (hereinafter, referred to as Compound Class SX₃₃₀₅).

A compound (L-13) wherein R³³ represents a chlorine atom, T represents a group represented by T2, R^{3b} represents a hydrogen atom, R^{3c} represents a hydrogen atom, and R¹ represents any substituents described in [Table L1] to [Table L3] (hereinafter, referred to as Compound Class SX₃₃₀₆).

A compound (L-13) wherein R³³ represents a chlorine atom, T represents a group represented by T2, R^{3b} represents CF₃, R^{3c} represents a hydrogen atom, and R¹ represents any substituents described in [Table L1] to [Table L3](hereinafter, referred to as Compound Class SX₃₃₀₇).

A compound (L-13) wherein R³³ represents a chlorine atom, T represents a group represented by T2, R^{3b} represents a chlorine atom, R^{3c} represents a hydrogen atom, and R¹ represents any substituents described in [Table L1] to [Table L3] (hereinafter, referred to as Compound Class SX₃₃₀₈).

A compound (L-13) wherein R³³ represents a chlorine atom, T represents a group represented by T2, R^{3b} represents a bromine atom, R^{3c} represents a hydrogen atom, and R¹ represents any substituents described in [Table L1] to [Table L3] (hereinafter, referred to as Compound Class SX₃₃₀₉).

A compound (L-13) wherein R³³ represents a chlorine atom, T represents a group represented by T2, R^{3b} represents an iodine atom, R^{3c} represents a hydrogen atom, and R¹ represents any substituents described in [Table L1] to [Table L3](hereinafter, referred to as Compound Class SX₃₃₁₀).

A compound (L-13) wherein R³³ represents a chlorine atom, T represents a group represented by T2, R^{3b} represents a hydrogen atom, R^{3c} represents CF₃, and R¹ represents any substituents described in [Table L1] to [Table L3](hereinafter, referred to as Compound Class SX₃₃₁₁).

A compound (L-13) wherein R³³ represents a chlorine atom, T represents a group represented by T2, R^{3b} represents a hydrogen atom, R^{3c} represents a chlorine atom, and R¹ represents any substituents described in [Table L1] to [Table L3] (hereinafter, referred to as Compound Class SX₃₃₁₂).

A compound (L-13) wherein R³³ represents a chlorine atom, T represents a group represented by T2, R^{3b} represents a hydrogen atom, R^{3c} represents a bromine atom, and R¹ represents any substituents described in [Table L1] to [Table L3] (hereinafter, referred to as Compound Class SX₃₃₁₃).

A compound (L-13) wherein R³³ represents a bromine atom, T represents a group represented by T2, R^{3b} represents a hydrogen atom, R^{3c} represents a hydrogen atom, and R¹ represents any substituents described in [Table L1] to [Table L3] (hereinafter, referred to as Compound Class SX₃₃₁₄).

A compound (L-13) wherein R³³ represents a bromine atom, T represents a group represented by T2, R^{3b} represents CF₃, R^{3c} represents a hydrogen atom, and R¹ represents any substituents described in [Table L1] to [Table L3](hereinafter, referred to as Compound Class SX₃₃₁₅).

A compound (L-13) wherein R³³ represents a bromine atom, T represents a group represented by T2, R^{3b} represents a chlorine atom, R^{3c} represents a hydrogen atom, and R¹ represents any substituents described in [Table L1] to [Table L3] (hereinafter, referred to as Compound Class SX₃₃₁₆).

A compound (L-13) wherein R³³ represents a bromine atom, T represents a group represented by T2, R^{3b} represents a bromine atom, R^{3c} represents a hydrogen atom, and R¹ represents any substituents described in [Table L1] to [Table L3] (hereinafter, referred to as Compound Class SX₃₃₁₇).

A compound (L-13) wherein R³³ represents a bromine atom, T represents a group represented by T2, R^{3b} represents an iodine atom, R^{3c} represents a hydrogen atom, and R¹ represents any substituents described in [Table L1] to [Table L3](hereinafter, referred to as Compound Class SX₃₃₁₈).

A compound (L-13) wherein R³³ represents a bromine atom, T represents a group represented by T2, R^{3b} represents a hydrogen atom, R^{3c} represents CF₃, and R¹ represents any substituents described in [Table L1] to [Table L3](hereinafter, referred to as Compound Class SX₃₃₁₉).

A compound (L-13) wherein R³³ represents a bromine atom, T represents a group represented by T2, R^{3b} represents a hydrogen atom, R^{3c} represents a chlorine atom, and R¹ represents any substituents described in [Table L1] to [Table L3] (hereinafter, referred to as Compound Class SX₃₃₂₀).

A compound (L-13) wherein R³³ represents a bromine atom, T represents a group represented by T2, R^{3b} represents a hydrogen atom, R^{3c} represents a bromine atom, and R¹ represents any substituents described in [Table L1] to [Table L3] (hereinafter, referred to as Compound Class SX₃₃₂₁).

A compound (L-13) wherein R³³ represents an iodine atom, T represents a group represented by T2, R^{3b} represents a hydrogen atom, R^{3c} represents a hydrogen atom, and R¹ represents any substituents described in [Table L1] to [Table L3] (hereinafter, referred to as Compound Class SX₃₃₂₂).

A compound (L-13) wherein R³³ represents an iodine atom, T represents a group represented by T2, R^{3b} represents CF₃, R^{3c} represents a hydrogen atom, and R¹ represents any substituents described in [Table L1] to [Table L3](hereinafter, referred to as Compound Class SX₃₃₂₃).

A compound (L-13) wherein R³³ represents an iodine atom, T represents a group represented by T2, R^{3b} represents a chlorine atom, R^{3c} represents a hydrogen atom, and R¹ represents any substituents described in [Table L1] to [Table L3] (hereinafter, referred to as Compound Class SX₃₃₂₄).

A compound (L-13) wherein R³³ represents an iodine atom, T represents a group represented by T2, R^{3b} represents a bromine atom, R^{3c} represents a hydrogen atom, and R¹ represents any substituents described in [Table L1] to [Table L3] (hereinafter, referred to as Compound Class SX₃₃₂₅).

A compound (L-13) wherein R³³ represents an iodine atom, T represents a group represented by T2, R^{3b} represents an iodine atom, R^{3c} represents a hydrogen atom, and R¹ represents any substituents described in [Table L1] to [Table L3](hereinafter, referred to as Compound Class SX₃₃₂₆).

A compound (L-13) wherein R³³ represents an iodine atom, T represents a group represented by T2, R^{3b} represents a hydrogen atom, R^{3c} represents CF₃, and R¹ represents any substituents described in [Table L1] to [Table L3](hereinafter, referred to as Compound Class SX₃₃₂₇).

A compound (L-13) wherein R³³ represents an iodine atom, T represents a group represented by T2, R^{3b} represents a hydrogen atom, R^{3c} represents a chlorine atom, and R¹ represents any substituents described in [Table L1] to [Table L3] (hereinafter, referred to as Compound Class SX₃₃₂₈).

A compound (L-13) wherein R³³ represents an iodine atom, T represents a group represented by T2, R^{3b} represents a hydrogen atom, R^{3c} represents a bromine atom, and R¹ represents any substituents described in [Table L1] to [Table L3] (hereinafter, referred to as Compound Class SX₃₃₂₉).

A compound (L-13) wherein R³³ represents a hydrogen atom, T represents a group represented by T3, R^{3b} represents a hydrogen atom, R^{3c} represents a hydrogen atom, and R¹ represents any substituents described in [Table L1] to [Table L3] (hereinafter, referred to as Compound Class SX₃₃₃₀).

A compound (L-13) wherein R³³ represents a hydrogen atom, T represents a group represented by T3, R^{3b} represents CF₃, R^{3c} represents a hydrogen atom, and R¹ represents any substituents described in [Table L1] to [Table L3](hereinafter, referred to as Compound Class SX₃₃₃₁).

A compound (L-13) wherein R³³ represents a hydrogen atom, T represents a group represented by T3, R^{3b} represents a chlorine atom, R^{3c} represents a hydrogen atom, and R¹ represents any substituents described in [Table L1] to [Table L3] (hereinafter, referred to as Compound Class SX₃₃₃₂).

A compound (L-13) wherein R³³ represents a hydrogen atom, T represents a group represented by T3, R^{3b} represents a bromine atom, R^{3c} represents a hydrogen atom, and R¹ represents any substituents described in [Table L1] to [Table L3] (hereinafter, referred to as Compound Class SX₃₃₃₃).

A compound (L-13) wherein R³³ represents a hydrogen atom, T represents a group represented by T3, R^{3b} represents an iodine atom, R^{3c} represents a hydrogen atom, and R¹ represents any substituents described in [Table L1] to [Table L3](hereinafter, referred to as Compound Class SX₃₃₃₄).

A compound (L-13) wherein R³³ represents a hydrogen atom, T represents a group represented by T3, R^{3b} represents a hydrogen atom, R^{3c} represents CF₃, and R¹ represents any substituents described in [Table L1] to [Table L3](hereinafter, referred to as Compound Class SX₃₃₃₅).

A compound (L-13) wherein R³³ represents a hydrogen atom, T represents a group represented by T3, R^{3b} represents a hydrogen atom, R^{3c} represents a chlorine atom, and R¹ represents any substituents described in [Table L1] to [Table L3] (hereinafter, referred to as Compound Class SX₃₃₃₆).

A compound (L-13) wherein R³³ represents a hydrogen atom, T represents a group represented by T3, R^{3b} represents a hydrogen atom, R^{3c} represents a bromine atom, and R¹ represents any substituents described in [Table L1] to [Table L3] (hereinafter, referred to as Compound Class SX₃₃₃₇).

A compound (L-13) wherein R³³ represents a fluorine atom, T represents a group represented by T3, R^{3b} represents a hydrogen atom, R^{3c} represents a hydrogen atom, and R¹ represents any substituents described in [Table L1] to [Table L3] (hereinafter, referred to as Compound Class SX₃₃₃₈).

A compound (L-13) wherein R³³ represents a fluorine atom, T represents a group represented by T3, R^{3b} represents CF₃, R^{3c} represents a hydrogen atom, and R¹ represents any substituents described in [Table L1] to [Table L3](hereinafter, referred to as Compound Class SX₃₃₃₉).

A compound (L-13) wherein R³³ represents a fluorine atom, T represents a group represented by T3, R^{3b} represents a chlorine atom, R^{3c} represents a hydrogen atom, and R¹ represents any substituents described in [Table L1] to [Table L3] (hereinafter, referred to as Compound Class SX₃₃₄₀).

A compound (L-13) wherein R³³ represents a fluorine atom, T represents a group represented by T3, R^{3b} represents a bromine atom, R^{3c} represents a hydrogen atom, and R¹ represents any substituents described in [Table L1] to [Table L3] (hereinafter, referred to as Compound Class SX₃₃₄₁).

A compound (L-13) wherein R³³ represents a fluorine atom, T represents a group represented by T3, R^{3b} represents an iodine atom, R^{3c} represents a hydrogen atom, and R¹ represents any substituents described in [Table L1] to [Table L3](hereinafter, referred to as Compound Class SX₃₃₄₂).

A compound (L-13) wherein R³³ represents a fluorine atom, T represents a group represented by T3, R^{3b} represents a hydrogen atom, R^{3c} represents CF₃, and R¹ represents any substituents described in [Table L1] to [Table L3](hereinafter, referred to as Compound Class SX₃₃₄₃).

A compound (L-13) wherein R³³ represents a fluorine atom, T represents a group represented by T3, R^{3b} represents a hydrogen atom, R^{3c} represents a chlorine atom, and R¹ represents any substituents described in [Table L1] to [Table L3] (hereinafter, referred to as Compound Class SX₃₃₄₄).

A compound (L-13) wherein R³³ represents a fluorine atom, T represents a group represented by T3, R^{3b} represents a hydrogen atom, R^{3c} represents a bromine atom, and R¹ represents any substituents described in [Table L1] to [Table L3] (hereinafter, referred to as Compound Class SX₃₃₄₅).

A compound (L-13) wherein R³³ represents a chlorine atom, T represents a group represented by T3, R^{3b} represents a hydrogen atom, R^{3c} represents a hydrogen atom, and R¹ represents any substituents described in [Table L1] to [Table L3] (hereinafter, referred to as Compound Class SX₃₃₄₆).

A compound (L-13) wherein R³³ represents a chlorine atom, T represents a group represented by T3, R^{3b} represents CF₃, R^{3c} represents a hydrogen atom, and R¹ represents any substituents described in [Table L1] to [Table L3](hereinafter, referred to as Compound Class SX₃₃₄₇).

A compound (L-13) wherein R³³ represents a chlorine atom, T represents a group represented by T3, R^{3b} represents a chlorine atom, R^{3c} represents a hydrogen atom, and R¹ represents any substituents described in [Table L1] to [Table L3] (hereinafter, referred to as Compound Class SX₃₃₄₈).

A compound (L-13) wherein R³³ represents a chlorine atom, T represents a group represented by T3, R^{3b} represents a bromine atom, R^{3c} represents a hydrogen atom, and R¹ represents any substituents described in [Table L1] to [Table L3] (hereinafter, referred to as Compound Class SX₃₃₄₉).

A compound (L-13) wherein R³³ represents a chlorine atom, T represents a group represented by T3, R^{3b} represents an iodine atom, R^{3c} represents a hydrogen atom, and R¹ represents any substituents described in [Table L1] to [Table L3](hereinafter, referred to as Compound Class SX₃₃₅₀).

A compound (L-13) wherein R³³ represents a chlorine atom, T represents a group represented by T3, R^{3b} represents a hydrogen atom, R^{3c} represents CF₃, and R¹ represents any substituents described in [Table L1] to [Table L3](hereinafter, referred to as Compound Class SX₃₃₅₁).

A compound (L-13) wherein R³³ represents a chlorine atom, T represents a group represented by T3, R^{3b} represents a hydrogen atom, R^{3c} represents a chlorine atom, and R¹ represents any substituents described in [Table L1] to [Table L3] (hereinafter, referred to as Compound Class SX₃₃₅₂).

A compound (L-13) wherein R³³ represents a chlorine atom, T represents a group represented by T3, R^{3b} represents a hydrogen atom, R^{3c} represents a bromine atom, and R¹ represents any substituents described in [Table L1] to [Table L3] (hereinafter, referred to as Compound Class SX₃₃₅₃).

A compound (L-13) wherein R³³ represents a bromine atom, T represents a group represented by T3, R^{3b} represents a hydrogen atom, R^{3c} represents a hydrogen atom, and R¹ represents any substituents described in [Table L1] to [Table L3] (hereinafter, referred to as Compound Class SX₃₃₅₄).

A compound (L-13) wherein R³³ represents a bromine atom, T represents a group represented by T3, R^{3b} represents CF₃, R^{3c} represents a hydrogen atom, and R¹ represents any substituents described in [Table L1] to [Table L3](hereinafter, referred to as Compound Class SX₃₃₅₅).

A compound (L-13) wherein R³³ represents a bromine atom, T represents a group represented by T3, R^{3b} represents a chlorine atom, R^{3c} represents a hydrogen atom, and R¹ represents any substituents described in [Table L1] to [Table L3] (hereinafter, referred to as Compound Class SX₃₃).

A compound (L-13) wherein R³³ represents a bromine atom, T represents a group represented by T3, R^{3b} represents a bromine atom, R^{3c} represents a hydrogen atom, and R¹ represents any substituents described in [Table L1] to [Table L3] (hereinafter, referred to as Compound Class SX₃₃₅₇).

A compound (L-13) wherein R³³ represents a bromine atom, T represents a group represented by T3, R^{3b} represents an iodine atom, R^{3c} represents a hydrogen atom, and R¹ represents any substituents described in [Table L1] to [Table L3](hereinafter, referred to as Compound Class SX₃₃₅₈).

A compound (L-13) wherein R³³ represents a bromine atom, T represents a group represented by T3, R^{3b} represents a hydrogen atom, R^{3c} represents CF₃, and R¹ represents any substituents described in [Table L1] to [Table L3](hereinafter, referred to as Compound Class SX₃₃₅₉).

A compound (L-13) wherein R³³ represents a bromine atom, T represents a group represented by T3, R^{3b} represents a hydrogen atom, R^{3c} represents a chlorine atom, and R¹ represents any substituents described in [Table L1] to [Table L3] (hereinafter, referred to as Compound Class SX₃₃₀).

A compound (L-13) wherein R³³ represents a bromine atom, T represents a group represented by T3, R^{3b} represents a hydrogen atom, R^{3c} represents a bromine atom, and R¹ represents any substituents described in [Table L1] to [Table L3] (hereinafter, referred to as Compound Class SX₃₃₆₁).

A compound (L-13) wherein R³³ represents an iodine atom, T represents a group represented by T3, R^{3b} represents a hydrogen atom, R^{3c} represents a hydrogen atom, and R¹ represents any substituents described in [Table L1] to [Table L3] (hereinafter, referred to as Compound Class SX₃₃₆₂).

A compound (L-13) wherein R³³ represents an iodine atom, T represents a group represented by T3, R^{3b} represents CF₃, R^{3c} represents a hydrogen atom, and R¹ represents any substituents described in [Table L1] to [Table L3](hereinafter, referred to as Compound Class SX₃₃₆₃).

A compound (L-13) wherein R³³ represents an iodine atom, T represents a group represented by T3, R^{3b} represents a chlorine atom, R^{3c} represents a hydrogen atom, and R¹ represents any substituents described in [Table L1] to [Table L3] (hereinafter, referred to as Compound Class SX₃₃₆₄).

A compound (L-13) wherein R³³ represents an iodine atom, T represents a group represented by T3, R^{3b} represents a bromine atom, R^{3c} represents a hydrogen atom, and R¹ represents any substituents described in [Table L1] to [Table L3] (hereinafter, referred to as Compound Class SX₃₃₆₅).

A compound (L-13) wherein R³³ represents an iodine atom, T represents a group represented by T3, R^{3b} represents an iodine atom, R^{3c} represents a hydrogen atom, and R¹ represents any substituents described in [Table L1] to [Table L3](hereinafter, referred to as Compound Class SX₃₃₆₆).

A compound (L-13) wherein R³³ represents an iodine atom, T represents a group represented by T3, R^{3b} represents a hydrogen atom, R^{3c} represents CF₃, and R¹ represents any substituents described in [Table L1] to [Table L3](hereinafter, referred to as Compound Class SX₃₃₆₇).

A compound (L-13) wherein R³³ represents an iodine atom, T represents a group represented by T3, R^{3b} represents a hydrogen atom, R^{3c} represents a chlorine atom, and R¹ represents any substituents described in [Table L1] to [Table L3] (hereinafter, referred to as Compound Class SX₃₃₆₈).

A compound (L-13) wherein R³³ represents an iodine atom, T represents a group represented by T3, R^{3b} represents a hydrogen atom, R^{3c} represents a bromine atom, and R¹ represents any substituents described in [Table L1] to [Table L3] (hereinafter, referred to as Compound Class SX₃₃₆₉).

A compound (L-12) wherein R³³ represents a hydrogen atom, T represents a group represented by T1, R¹ represents CF₃, R^{3c} represents a hydrogen atom, and R^{3b} represents any substituents described in [Table L4] to [Table L12](hereinafter, referred to as Compound Class SX₃₃₇₀).

A compound (L-12) wherein R³³ represents a hydrogen atom, T represents a group represented by T1, R¹ represents C₂F5, R^{3c} represents a hydrogen atom, and R^{3b} represents any substituents described in [Table L4] to [Table L12](hereinafter, referred to as Compound Class SX₃₃₇₁).

A compound (L-12) wherein R³³ represents a hydrogen atom, T represents a group represented by T1, R¹ represents OCF₃, R^{3c} represents a hydrogen atom, and R^{3b} represents any substituents described in [Table L4] to [Table L12](hereinafter, referred to as Compound Class SX₃₃₇₂).

A compound (L-12) wherein R³³ represents a hydrogen atom, T represents a group represented by T1, R¹ represents SCF₃, R^{3c} represents a hydrogen atom, and R^{3b} represents any substituents described in [Table L4] to [Table L12](hereinafter, referred to as Compound Class SX₃₃₇₃).

A compound (L-12) wherein R³³ represents a hydrogen atom, T represents a group represented by T1, R¹ represents S(O)CF₃, R^{3c} represents a hydrogen atom, and R^{3b} represents any substituents described in [Table L4] to [Table L12](hereinafter, referred to as Compound Class SX₃₃₇₄).

A compound (L-12) wherein R³³ represents a hydrogen atom, T represents a group represented by T1, R¹ represents S(O)₂CF₃, R^{3c} represents a hydrogen atom, and R^{3b} represents any substituents described in [Table L4] to [Table L12](hereinafter, referred to as Compound Class SX₃₃₇₅).

A compound (L-12) wherein R³³ represents a fluorine atom, T represents a group represented by T1, R¹ represents CF₃, R^{3c} represents a hydrogen atom, and R^{3b} represents any substituents described in [Table L4] to [Table L12](hereinafter, referred to as Compound Class SX₃₃₇₆).

A compound (L-12) wherein R³³ represents a fluorine atom, T represents a group represented by T1, R¹ represents C₂F5, R^{3c} represents a hydrogen atom, and R^{3b} represents any substituents described in [Table L4] to [Table L12](hereinafter, referred to as Compound Class SX₃₃₇₇).

A compound (L-12) wherein R³³ represents a fluorine atom, T represents a group represented by T1, R¹ represents OCF₃, R^{3c} represents a hydrogen atom, and R^{3b} represents any substituents described in [Table L4] to [Table L12](hereinafter, referred to as Compound Class SX₃₃₇₈).

A compound (L-12) wherein R³³ represents a fluorine atom, T represents a group represented by T1, R¹ represents SCF₃, R^{3c} represents a hydrogen atom, and R^{3b} represents any substituents described in [Table L4] to [Table L12](hereinafter, referred to as Compound Class SX₃₃₇₉).

A compound (L-12) wherein R³³ represents a fluorine atom, T represents a group represented by T1, R¹ represents S(O)CF₃, R^{3c} represents a hydrogen atom, and R^{3b} represents any substituents described in [Table L4] to [Table L12](hereinafter, referred to as Compound Class SX₃₃₈₀).

A compound (L-12) wherein R³³ represents a fluorine atom, T represents a group represented by T1, R¹ represents S(O)₂CF₃, R^{3c} represents a hydrogen atom, and R^{3b} represents any substituents described in [Table L4] to [Table L12](hereinafter, referred to as Compound Class SX₃₃₈₁).

A compound (L-12) wherein R³³ represents a chlorine atom, T represents a group represented by T1, R¹ represents CF₃, R^{3c} represents a hydrogen atom, and R^{3b} represents any substituents described in [Table L4] to [Table L12](hereinafter, referred to as Compound Class SX₃₃₈₂).

A compound (L-12) wherein R³³ represents a chlorine atom, T represents a group represented by T1, R¹ represents C₂F5, R^{3c} represents a hydrogen atom, and R^{3b} represents any substituents described in [Table L4] to [Table L12](hereinafter, referred to as Compound Class SX₃₃₈₃).

A compound (L-12) wherein R³³ represents a chlorine atom, T represents a group represented by T1, R¹ represents OCF₃, R^{3c} represents a hydrogen atom, and R^{3b} represents any substituents described in [Table L4] to [Table L12](hereinafter, referred to as Compound Class SX₃₃₈₄).

A compound (L-12) wherein R³³ represents a chlorine atom, T represents a group represented by T1, R¹ represents SCF₃, R^{3c} represents a hydrogen atom, and R^{3b} represents any substituents described in [Table L4] to [Table L12](hereinafter, referred to as Compound Class SX₃₃₈₅).

A compound (L-12) wherein R³³ represents a chlorine atom, T represents a group represented by T1, R¹ represents S(O)CF₃, R^{3c} represents a hydrogen atom, and R^{3b} represents any substituents described in [Table L4] to [Table L12](hereinafter, referred to as Compound Class SX₃₃₈₆).

A compound (L-12) wherein R³³ represents a chlorine atom, T represents a group represented by T1, R¹ represents S(O)₂CF₃, R^{3c} represents a hydrogen atom, and R^{3b} represents any substituents described in [Table L4] to [Table L12](hereinafter, referred to as Compound Class SX₃₃₈₇).

A compound (L-12) wherein R³³ represents a bromine atom, T represents a group represented by T1, R¹ represents CF₃, R^{3c} represents a hydrogen atom, and R^{3b} represents any substituents described in [Table L4] to [Table L12](hereinafter, referred to as Compound Class SX₃₃₈₈).

A compound (L-12) wherein R³³ represents a bromine atom, T represents a group represented by T1, R¹ represents C₂F5, R^{3c} represents a hydrogen atom, and R^{3b} represents any substituents described in [Table L4] to [Table L12](hereinafter, referred to as Compound Class SX₃₃₈₉).

A compound (L-12) wherein R³³ represents a bromine atom, T represents a group represented by T1, R¹ represents OCF₃, R^{3c} represents a hydrogen atom, and R^{3b} represents any substituents described in [Table L4] to [Table L12](hereinafter, referred to as Compound Class SX₃₃₉₀).

A compound (L-12) wherein R³³ represents a bromine atom, T represents a group represented by T1, R¹ represents SCF₃, R^{3c} represents a hydrogen atom, and R^{3b} represents any substituents described in [Table L4] to [Table L12](hereinafter, referred to as Compound Class SX₃₃₉₁).

A compound (L-12) wherein R³³ represents a bromine atom, T represents a group represented by T1, R¹ represents S(O)CF₃, R^{3c} represents a hydrogen atom, and R^{3b} represents any substituents described in [Table L4] to [Table L12](hereinafter, referred to as Compound Class SX₃₃₉₂).

A compound (L-12) wherein R³³ represents a bromine atom, T represents a group represented by T1, R¹ represents S(O)₂CF₃, R^{3c} represents a hydrogen atom, and R^{3b} represents any substituents described in [Table L4] to [Table L12](hereinafter, referred to as Compound Class SX₃₃₉₃).

A compound (L-12) wherein R³³ represents an iodine atom, T represents a group represented by T1, R¹ represents CF₃, R^{3c} represents a hydrogen atom, and R^{3b} represents any substituents described in [Table L4] to [Table L12](hereinafter, referred to as Compound Class SX₃₃₉₄).

A compound (L-12) wherein R³³ represents an iodine atom, T represents a group represented by T1, R¹ represents C₂F5, R^{3c} represents a hydrogen atom, and R^{3b} represents any substituents described in [Table L4] to [Table L12](hereinafter, referred to as Compound Class SX₃₃₉₅).

A compound (L-12) wherein R³³ represents an iodine atom, T represents a group represented by T1, R¹ represents OCF₃, R^{3c} represents a hydrogen atom, and R^{3b} represents any substituents described in [Table L4] to [Table L12](hereinafter, referred to as Compound Class SX₃₃₉₆).

A compound (L-12) wherein R³³ represents an iodine atom, T represents a group represented by T1, R¹ represents SCF₃, R^{3c} represents a hydrogen atom, and R^{3b} represents any substituents described in [Table L4] to [Table L12](hereinafter, referred to as Compound Class SX₃₃₉₇).

A compound (L-12) wherein R³³ represents an iodine atom, T represents a group represented by T1, R¹ represents S(O)CF₃, R^{3c} represents a hydrogen atom, and R^{3b} represents any substituents described in [Table L4] to [Table L12](hereinafter, referred to as Compound Class SX₃₃₉₈).

A compound (L-12) wherein R³³ represents an iodine atom, T represents a group represented by T1, R¹ represents S(O)₂CF₃, R^{3c} represents a hydrogen atom, and R^{3b} represents any substituents described in [Table L4] to [Table L12](hereinafter, referred to as Compound Class SX₃₃₉₉).

A compound (L-12) wherein R³³ represents a hydrogen atom, T represents a group represented by T2, R¹ represents CF₃, R^{3c} represents a hydrogen atom, and R^{3b} represents any substituents described in [Table L4] to [Table L12](hereinafter, referred to as Compound Class SX₃₄₀₀).

A compound (L-12) wherein R³³ represents a hydrogen atom, T represents a group represented by T2, R¹ represents C₂F5, R^{3c} represents a hydrogen atom, and R^{3b} represents any substituents described in [Table L4] to [Table L12](hereinafter, referred to as Compound Class SX₃₄₀₁).

A compound (L-12) wherein R³³ represents a hydrogen atom, T represents a group represented by T2, R¹ represents OCF₃, R^{3c} represents a hydrogen atom, and R^{3b} represents any substituents described in [Table L4] to [Table L12](hereinafter, referred to as Compound Class SX₃₄₀₂).

A compound (L-12) wherein R³³ represents a hydrogen atom, T represents a group represented by T2, R¹ represents SCF₃, R^{3c} represents a hydrogen atom, and R^{3b} represents any substituents described in [Table L4] to [Table L12](hereinafter, referred to as Compound Class SX₃₄₀₃).

A compound (L-12) wherein R³³ represents a hydrogen atom, T represents a group represented by T2, R¹ represents S(O)CF₃, R^{3c} represents a hydrogen atom, and R^{3b} represents any substituents described in [Table L4] to [Table L12](hereinafter, referred to as Compound Class SX₃₄₀₄).

A compound (L-12) wherein R³³ represents a hydrogen atom, T represents a group represented by T2, R¹ represents S(O)₂CF₃, R^{3c} represents a hydrogen atom, and R^{3b} represents any substituents described in [Table L4] to [Table L12](hereinafter, referred to as Compound Class SX₃₄₀₅).

A compound (L-12) wherein R³³ represents a fluorine atom, T represents a group represented by T2, R¹ represents CF₃, R^{3c} represents a hydrogen atom, and R^{3b} represents any substituents described in [Table L4] to [Table L12](hereinafter, referred to as Compound Class SX₃₄₀₆).

A compound (L-12) wherein R³³ represents a fluorine atom, T represents a group represented by T2, R¹ represents C₂F5, R^{3c} represents a hydrogen atom, and R^{3b} represents any substituents described in [Table L4] to [Table L12](hereinafter, referred to as Compound Class SX₃₄₀₇).

A compound (L-12) wherein R³³ represents a fluorine atom, T represents a group represented by T2, R¹ represents OCF₃, R^{3c} represents a hydrogen atom, and R^{3b} represents any substituents described in [Table L4] to [Table L12](hereinafter, referred to as Compound Class SX₃₄₀₈).

A compound (L-12) wherein R³³ represents a fluorine atom, T represents a group represented by T2, R¹ represents SCF₃, R^{3c} represents a hydrogen atom, and R^{3b} represents any substituents described in [Table L4] to [Table L12](hereinafter, referred to as Compound Class SX₃₄₀₉).

A compound (L-12) wherein R³³ represents a fluorine atom, T represents a group represented by T2, R¹ represents S(O)CF₃, R^{3c} represents a hydrogen atom, and R^{3b} represents any substituents described in [Table L4] to [Table L12](hereinafter, referred to as Compound Class SX₃₄₁₀).

A compound (L-12) wherein R³³ represents a fluorine atom, T represents a group represented by T2, R¹ represents S(O)₂CF₃, R^{3c} represents a hydrogen atom, and R^{3b} represents any substituents described in [Table L4] to [Table L12](hereinafter, referred to as Compound Class SX₃₄₁₁).

A compound (L-12) wherein R³³ represents a chlorine atom, T represents a group represented by T2, R¹ represents CF₃, R^{3c} represents a hydrogen atom, and R^{3b} represents any substituents described in [Table L4] to [Table L12](hereinafter, referred to as Compound Class SX₃₄₁₂).

A compound (L-12) wherein R³³ represents a chlorine atom, T represents a group represented by T2, R¹ represents C₂F5, R^{3c} represents a hydrogen atom, and R^{3b} represents any substituents described in [Table L4] to [Table L12](hereinafter, referred to as Compound Class SX₃₄₁₃).

A compound (L-12) wherein R³³ represents a chlorine atom, T represents a group represented by T2, R¹ represents OCF₃, R^{3c} represents a hydrogen atom, and R^{3b} represents any substituents described in [Table L4] to [Table L12](hereinafter, referred to as Compound Class SX₃₄₁₄).

A compound (L-12) wherein R³³ represents a chlorine atom, T represents a group represented by T2, R¹ represents SCF₃, R^{3c} represents a hydrogen atom, and R^{3b} represents any substituents described in [Table L4] to [Table L12](hereinafter, referred to as Compound Class SX₃₄₁₅).

A compound (L-12) wherein R³³ represents a chlorine atom, T represents a group represented by T2, R¹ represents S(O)CF₃, R^{3c} represents a hydrogen atom, and R^{3b} represents any substituents described in [Table L4] to [Table L12](hereinafter, referred to as Compound Class SX₃₄₁₆).

A compound (L-12) wherein R³³ represents a chlorine atom, T represents a group represented by T2, R¹ represents S(O)₂CF₃, R^{3c} represents a hydrogen atom, and R^{3b} represents any substituents described in [Table L4] to [Table L12](hereinafter, referred to as Compound Class SX₃₄₁₇).

A compound (L-12) wherein R³³ represents a bromine atom, T represents a group represented by T2, R¹ represents CF₃, R^{3c} represents a hydrogen atom, and R^{3b} represents any substituents described in [Table L4] to [Table L12](hereinafter, referred to as Compound Class SX₃₄₁₈).

A compound (L-12) wherein R³³ represents a bromine atom, T represents a group represented by T2, R¹ represents C₂F5, R^{3c} represents a hydrogen atom, and R^{3b} represents any substituents described in [Table L4] to [Table L12](hereinafter, referred to as Compound Class SX₃₄₁₉).

A compound (L-12) wherein R³³ represents a bromine atom, T represents a group represented by T2, R¹ represents OCF₃, R^{3c} represents a hydrogen atom, and R^{3b} represents any substituents described in [Table L4] to [Table L12](hereinafter, referred to as Compound Class SX₃₄₂₀).

A compound (L-12) wherein R³³ represents a bromine atom, T represents a group represented by T2, R¹ represents SCF₃, R^{3c} represents a hydrogen atom, and R^{3b} represents any substituents described in [Table L4] to [Table L12](hereinafter, referred to as Compound Class SX₃₄₂₁).

A compound (L-12) wherein R³³ represents a bromine atom, T represents a group represented by T2, R¹ represents S(O)CF₃, R^{3c} represents a hydrogen atom, and R^{3b} represents any substituents described in [Table L4] to [Table L12](hereinafter, referred to as Compound Class SX₃₄₂₂).

A compound (L-12) wherein R³³ represents a bromine atom, T represents a group represented by T2, R¹ represents S(O)₂CF₃, R^{3c} represents a hydrogen atom, and R^{3b} represents any substituents described in [Table L4] to [Table L12](hereinafter, referred to as Compound Class SX₃₄₂₃).

A compound (L-12) wherein R³³ represents an iodine atom, T represents a group represented by T2, R¹ represents CF₃, R^{3c} represents a hydrogen atom, and R^{3b} represents any substituents described in [Table L4] to [Table L12](hereinafter, referred to as Compound Class SX₃₄₂₄).

A compound (L-12) wherein R³³ represents an iodine atom, T represents a group represented by T2, R¹ represents C₂F5, R^{3c} represents a hydrogen atom, and R^{3b} represents any substituents described in [Table L4] to [Table L12](hereinafter, referred to as Compound Class SX₃₄₂₅).

A compound (L-12) wherein R³³ represents an iodine atom, T represents a group represented by T2, R¹ represents OCF₃, R^{3c} represents a hydrogen atom, and R^{3b} represents any substituents described in [Table L4] to [Table L12](hereinafter, referred to as Compound Class SX₃₄₂₆).

A compound (L-12) wherein R³³ represents an iodine atom, T represents a group represented by T2, R¹ represents SCF₃, R^{3c} represents a hydrogen atom, and R^{3b} represents any substituents described in [Table L4] to [Table L12](hereinafter, referred to as Compound Class SX₃₄₂₇).

A compound (L-12) wherein R³³ represents an iodine atom, T represents a group represented by T2, R¹ represents S(O)CF₃, R^{3c} represents a hydrogen atom, and R^{3b} represents any substituents described in [Table L4] to [Table L12](hereinafter, referred to as Compound Class SX₃₄₂₈).

A compound (L-12) wherein R³³ represents an iodine atom, T represents a group represented by T2, R¹ represents S(O)₂CF₃, R^{3c} represents a hydrogen atom, and R^{3b} represents any substituents described in [Table L4] to [Table L12](hereinafter, referred to as Compound Class SX₃₄₂₉).

A compound (L-12) wherein R³³ represents a hydrogen atom, T represents a group represented by T3, R¹ represents CF₃, R^{3c} represents a hydrogen atom, and R^{3b} represents any substituents described in [Table L4] to [Table L12](hereinafter, referred to as Compound Class SX₃₄₃₀).

A compound (L-12) wherein R³³ represents a hydrogen atom, T represents a group represented by T3, R¹ represents C₂F5, R^{3c} represents a hydrogen atom, and R^{3b} represents any substituents described in [Table L4] to [Table L12](hereinafter, referred to as Compound Class SX₃₄₃₁).

A compound (L-12) wherein R³³ represents a hydrogen atom, T represents a group represented by T3, R¹ represents OCF₃, R^{3c} represents a hydrogen atom, and R^{3b} represents any substituents described in [Table L4] to [Table L12](hereinafter, referred to as Compound Class SX₃₄₃₂).

A compound (L-12) wherein R³³ represents a hydrogen atom, T represents a group represented by T3, R¹ represents SCF₃, R^{3c} represents a hydrogen atom, and R^{3b} represents any substituents described in [Table L4] to [Table L12](hereinafter, referred to as Compound Class SX₃₄₃₃).

A compound (L-12) wherein R³³ represents a hydrogen atom, T represents a group represented by T3, R¹ represents S(O)CF₃, R^{3c} represents a hydrogen atom, and R^{3b} represents any substituents described in [Table L4] to [Table L12](hereinafter, referred to as Compound Class SX₃₄₃₄).

A compound (L-12) wherein R³³ represents a hydrogen atom, T represents a group represented by T3, R¹ represents S(O)₂CF₃, R^{3c} represents a hydrogen atom, and R^{3b} represents any substituents described in [Table L4] to [Table L12](hereinafter, referred to as Compound Class SX₃₄₃₅).

A compound (L-12) wherein R³³ represents a fluorine atom, T represents a group represented by T3, R¹ represents CF₃, R^{3c} represents a hydrogen atom, and R^{3b} represents any substituents described in [Table L4] to [Table L12](hereinafter, referred to as Compound Class SX₃₄₃₆).

A compound (L-12) wherein R³³ represents a fluorine atom, T represents a group represented by T3, R¹ represents C₂F5, R^{3c} represents a hydrogen atom, and R^{3b} represents any substituents described in [Table L4] to [Table L12](hereinafter, referred to as Compound Class SX₃₄₃₇).

A compound (L-12) wherein R³³ represents a fluorine atom, T represents a group represented by T3, R¹ represents OCF₃, R^{3c} represents a hydrogen atom, and R^{3b} represents any substituents described in [Table L4] to [Table L12](hereinafter, referred to as Compound Class SX₃₄₃₈).

A compound (L-12) wherein R³³ represents a fluorine atom, T represents a group represented by T3, R¹ represents SCF₃, R^{3c} represents a hydrogen atom, and R^{3b} represents any substituents described in [Table L4] to [Table L12](hereinafter, referred to as Compound Class SX₃₄₃₉).

A compound (L-12) wherein R³³ represents a fluorine atom, T represents a group represented by T3, R¹ represents S(O)CF₃, R^{3c} represents a hydrogen atom, and R^{3b} represents any substituents described in [Table L4] to [Table L12](hereinafter, referred to as Compound Class SX₃₄₄₀).

A compound (L-12) wherein R³³ represents a fluorine atom, T represents a group represented by T3, R¹ represents S(O)₂CF₃, R^{3c} represents a hydrogen atom, and R^{3b} represents any substituents described in [Table L4] to [Table L12](hereinafter, referred to as Compound Class SX₃₄₄₁).

A compound (L-12) wherein R³³ represents a chlorine atom, T represents a group represented by T3, R¹ represents CF₃, R^{3c} represents a hydrogen atom, and R^{3b} represents any substituents described in [Table L4] to [Table L12](hereinafter, referred to as Compound Class SX₃₄₄₂).

A compound (L-12) wherein R³³ represents a chlorine atom, T represents a group represented by T3, R¹ represents C₂F5, R^{3c} represents a hydrogen atom, and R^{3b} represents any substituents described in [Table L4] to [Table L12](hereinafter, referred to as Compound Class SX₃₄₄₃).

A compound (L-12) wherein R³³ represents a chlorine atom, T represents a group represented by T3, R¹ represents OCF₃, R^{3c} represents a hydrogen atom, and R^{3b} represents any substituents described in [Table L4] to [Table L12](hereinafter, referred to as Compound Class SX₃₄₄₄).

A compound (L-12) wherein R³³ represents a chlorine atom, T represents a group represented by T3, R¹ represents SCF₃, R^{3c} represents a hydrogen atom, and R^{3b} represents any substituents described in [Table L4] to [Table L12](hereinafter, referred to as Compound Class SX₃₄₄₅).

A compound (L-12) wherein R³³ represents a chlorine atom, T represents a group represented by T3, R¹ represents S(O)CF₃, R^{3c} represents a hydrogen atom, and R^{3b} represents any substituents described in [Table L4] to [Table L12](hereinafter, referred to as Compound Class SX₃₄₄₆).

A compound (L-12) wherein R³³ represents a chlorine atom, T represents a group represented by T3, R¹ represents S(O)₂CF₃, R^{3c} represents a hydrogen atom, and R^{3b} represents any substituents described in [Table L4] to [Table L12](hereinafter, referred to as Compound Class SX₃₄₄₇).

A compound (L-12) wherein R³³ represents a bromine atom, T represents a group represented by T3, R¹ represents CF₃, R^{3c} represents a hydrogen atom, and R^{3b} represents any substituents described in [Table L4] to [Table L12](hereinafter, referred to as Compound Class SX₃₄₄₈).

A compound (L-12) wherein R³³ represents a bromine atom, T represents a group represented by T3, R¹ represents C₂F5, R^{3c} represents a hydrogen atom, and R^{3b} represents any substituents described in [Table L4] to [Table L12](hereinafter, referred to as Compound Class SX₃₄₄₉).

A compound (L-12) wherein R³³ represents a bromine atom, T represents a group represented by T3, R¹ represents OCF₃, R^{3c} represents a hydrogen atom, and R^{3b} represents any substituents described in [Table L4] to [Table L12](hereinafter, referred to as Compound Class SX₃₄₅₀).

A compound (L-12) wherein R³³ represents a bromine atom, T represents a group represented by T3, R¹ represents SCF₃, R^{3c} represents a hydrogen atom, and R^{3b} represents any substituents described in [Table L4] to [Table L12](hereinafter, referred to as Compound Class SX₃₄₅₁).

A compound (L-12) wherein R³³ represents a bromine atom, T represents a group represented by T3, R¹ represents S(O)CF₃, R^{3c} represents a hydrogen atom, and R^{3b} represents any substituents described in [Table L4] to [Table L12](hereinafter, referred to as Compound Class SX₃₄₅₂).

A compound (L-12) wherein R³³ represents a bromine atom, T represents a group represented by T3, R¹ represents S(O)₂CF₃, R^{3c} represents a hydrogen atom, and R^{3b} represents any substituents described in [Table L4] to [Table L12](hereinafter, referred to as Compound Class SX₃₄₅₃).

A compound (L-12) wherein R³³ represents an iodine atom, T represents a group represented by T3, R¹ represents CF₃, R^{3c} represents a hydrogen atom, and R^{3b} represents any substituents described in [Table L4] to [Table L12](hereinafter, referred to as Compound Class SX₃₄₅₄).

A compound (L-12) wherein R³³ represents an iodine atom, T represents a group represented by T3, R¹ represents C₂F5, R^{3c} represents a hydrogen atom, and R^{3b} represents any substituents described in [Table L4] to [Table L12](hereinafter, referred to as Compound Class SX₃₄₅₅).

A compound (L-12) wherein R³³ represents an iodine atom, T represents a group represented by T3, R¹ represents OCF₃, R^{3c} represents a hydrogen atom, and R^{3b} represents any substituents described in [Table L4] to [Table L12](hereinafter, referred to as Compound Class SX₃₄₅₆).

A compound (L-12) wherein R³³ represents an iodine atom, T represents a group represented by T3, R¹ represents SCF₃, R^{3c} represents a hydrogen atom, and R^{3b} represents any substituents described in [Table L4] to [Table L12](hereinafter, referred to as Compound Class SX₃₄₅₇).

A compound (L-12) wherein R³³ represents an iodine atom, T represents a group represented by T3, R¹ represents S(O)CF₃, R^{3c} represents a hydrogen atom, and R^{3b} represents any substituents described in [Table L4] to [Table L12](hereinafter, referred to as Compound Class SX₃₄₅₈).

A compound (L-12) wherein R³³ represents an iodine atom, T represents a group represented by T3, R¹ represents S(O)₂CF₃, R^{3c} represents a hydrogen atom, and R^{3b} represents any substituents described in [Table L4] to [Table L12](hereinafter, referred to as Compound Class SX₃₄₅₉).

A compound (L-13) wherein R³³ represents a hydrogen atom, T represents a group represented by T1, R¹ represents CF₃, R^{3c} represents a hydrogen atom, and R^{3b} represents any substituents described in [Table L4] to [Table L12](hereinafter, referred to as Compound Class SX₃₄₆₀).

A compound (L-13) wherein R³³ represents a hydrogen atom, T represents a group represented by T1, R¹ represents C₂F5, R^{3c} represents a hydrogen atom, and R^{3b} represents any substituents described in [Table L4] to [Table L12](hereinafter, referred to as Compound Class SX₃₄₆₁).

A compound (L-13) wherein R³³ represents a hydrogen atom, T represents a group represented by T1, R¹ represents OCF₃, R^{3c} represents a hydrogen atom, and R^{3b} represents any substituents described in [Table L4] to [Table L12](hereinafter, referred to as Compound Class SX₃₄₆₂).

A compound (L-13) wherein R³³ represents a hydrogen atom, T represents a group represented by T1, R¹ represents SCF₃, R^{3c} represents a hydrogen atom, and R^{3b} represents any substituents described in [Table L4] to [Table L12](hereinafter, referred to as Compound Class SX₃₄₆₃).

A compound (L-13) wherein R³³ represents a hydrogen atom, T represents a group represented by T1, R¹ represents S(O)CF₃, R^{3c} represents a hydrogen atom, and R^{3b} represents any substituents described in [Table L4] to [Table L12](hereinafter, referred to as Compound Class SX₃₄₆₄).

A compound (L-13) wherein R³³ represents a hydrogen atom, T represents a group represented by T1, R¹ represents S(O)₂CF₃, R^{3c} represents a hydrogen atom, and R^{3b} represents any substituents described in [Table L4] to [Table L12](hereinafter, referred to as Compound Class SX₃₄₆₅).

A compound (L-13) wherein R³³ represents a fluorine atom, T represents a group represented by T1, R¹ represents CF₃, R^{3c} represents a hydrogen atom, and R^{3b} represents any substituents described in [Table L4] to [Table L12](hereinafter, referred to as Compound Class SX₃₄₆₆).

A compound (L-13) wherein R³³ represents a fluorine atom, T represents a group represented by T1, R¹ represents C₂F5, R^{3c} represents a hydrogen atom, and R^{3b} represents any substituents described in [Table L4] to [Table L12](hereinafter, referred to as Compound Class SX₃₄₆₇).

A compound (L-13) wherein R³³ represents a fluorine atom, T represents a group represented by T1, R¹ represents OCF₃, R^{3c} represents a hydrogen atom, and R^{3b} represents any substituents described in [Table L4] to [Table L12](hereinafter, referred to as Compound Class SX₃₄₆₈).

A compound (L-13) wherein R³³ represents a fluorine atom, T represents a group represented by T1, R¹ represents SCF₃, R^{3c} represents a hydrogen atom, and R^{3b} represents any substituents described in [Table L4] to [Table L12](hereinafter, referred to as Compound Class SX₃₄₆₉).

A compound (L-13) wherein R³³ represents a fluorine atom, T represents a group represented by T1, R¹ represents S(O)CF₃, R^{3c} represents a hydrogen atom, and R^{3b} represents any substituents described in [Table L4] to [Table L12](hereinafter, referred to as Compound Class SX₃₄₇₀).

A compound (L-13) wherein R³³ represents a fluorine atom, T represents a group represented by T1, R¹ represents S(O)₂CF₃, R^{3c} represents a hydrogen atom, and R^{3b} represents any substituents described in [Table L4] to [Table L12](hereinafter, referred to as Compound Class SX₃₄₇₁).

A compound (L-13) wherein R³³ represents a chlorine atom, T represents a group represented by T1, R¹ represents CF₃, R^{3c} represents a hydrogen atom, and R^{3b} represents any substituents described in [Table L4] to [Table L12](hereinafter, referred to as Compound Class SX₃₄₇₂).

A compound (L-13) wherein R³³ represents a chlorine atom, T represents a group represented by T1, R¹ represents C₂F5, R^{3c} represents a hydrogen atom, and R^{3b} represents any substituents described in [Table L4] to [Table L12](hereinafter, referred to as Compound Class SX₃₄₇₃).

A compound (L-13) wherein R³³ represents a chlorine atom, T represents a group represented by T1, R¹ represents OCF₃, R^{3c} represents a hydrogen atom, and R^{3b} represents any substituents described in [Table L4] to [Table L12](hereinafter, referred to as Compound Class SX₃₄₇₄).

A compound (L-13) wherein R³³ represents a chlorine atom, T represents a group represented by T1, R¹ represents SCF₃, R^{3c} represents a hydrogen atom, and R^{3b} represents any substituents described in [Table L4] to [Table L12](hereinafter, referred to as Compound Class SX₃₄₇₅).

A compound (L-13) wherein R³³ represents a chlorine atom, T represents a group represented by T1, R¹ represents S(O)CF₃, R^{3c} represents a hydrogen atom, and R^{3b} represents any substituents described in [Table L4] to [Table L12](hereinafter, referred to as Compound Class SX₃₄₇₆).

A compound (L-13) wherein R³³ represents a chlorine atom, T represents a group represented by T1, R¹ represents S(O)₂CF₃, R^{3c} represents a hydrogen atom, and R^{3b} represents any substituents described in [Table L4] to [Table L12](hereinafter, referred to as Compound Class SX₃₄₇₇).

A compound (L-13) wherein R³³ represents a bromine atom, T represents a group represented by T1, R¹ represents CF₃, R^{3c} represents a hydrogen atom, and R^{3b} represents any substituents described in [Table L4] to [Table L12](hereinafter, referred to as Compound Class SX₃₄₇₈).

A compound (L-13) wherein R³³ represents a bromine atom, T represents a group represented by T1, R¹ represents C₂F5, R^{3c} represents a hydrogen atom, and R^{3b} represents any substituents described in [Table L4] to [Table L12](hereinafter, referred to as Compound Class SX₃₄₇₉).

A compound (L-13) wherein R³³ represents a bromine atom, T represents a group represented by T1, R¹ represents OCF₃, R^{3c} represents a hydrogen atom, and R^{3b} represents any substituents described in [Table L4] to [Table L12](hereinafter, referred to as Compound Class SX₃₄₈₀).

A compound (L-13) wherein R³³ represents a bromine atom, T represents a group represented by T1, R¹ represents SCF₃, R^{3c} represents a hydrogen atom, and R^{3b} represents any substituents described in [Table L4] to [Table L12](hereinafter, referred to as Compound Class SX₃₄₈₁).

A compound (L-13) wherein R³³ represents a bromine atom, T represents a group represented by T1, R¹ represents S(O)CF₃, R^{3c} represents a hydrogen atom, and R^{3b} represents any substituents described in [Table L4] to [Table L12](hereinafter, referred to as Compound Class SX₃₄₂).

A compound (L-13) wherein R³³ represents a bromine atom, T represents a group represented by T1, R¹ represents S(O)₂CF₃, R^{3c} represents a hydrogen atom, and R^{3b} represents any substituents described in [Table L4] to [Table L12](hereinafter, referred to as Compound Class SX₃₄₈₃).

A compound (L-13) wherein R³³ represents an iodine atom, T represents a group represented by T1, R¹ represents CF₃, R^{3c} represents a hydrogen atom, and R^{3b} represents any substituents described in [Table L4] to [Table L12](hereinafter, referred to as Compound Class SX₃₄₈₄).

A compound (L-13) wherein R³³ represents an iodine atom, T represents a group represented by T1, R¹ represents C₂F5, R^{3c} represents a hydrogen atom, and R^{3b} represents any substituents described in [Table L4] to [Table L12](hereinafter, referred to as Compound Class SX₃₄₈₅).

A compound (L-13) wherein R³³ represents an iodine atom, T represents a group represented by T1, R¹ represents OCF₃, R^{3c} represents a hydrogen atom, and R^{3b} represents any substituents described in [Table L4] to [Table L12](hereinafter, referred to as Compound Class SX₃₄₈₆).

A compound (L-13) wherein R³³ represents an iodine atom, T represents a group represented by T1, R¹ represents SCF₃, R^{3c} represents a hydrogen atom, and R^{3b} represents any substituents described in [Table L4] to [Table L12](hereinafter, referred to as Compound Class SX₃₄₈₇).

A compound (L-13) wherein R³³ represents an iodine atom, T represents a group represented by T1, R¹ represents S(O)CF₃, R^{3c} represents a hydrogen atom, and R^{3b} represents any substituents described in [Table L4] to [Table L12](hereinafter, referred to as Compound Class SX₃₄₈₈).

A compound (L-13) wherein R³³ represents an iodine atom, T represents a group represented by T1, R¹ represents S(O)₂CF₃, R^{3c} represents a hydrogen atom, and R^{3b} represents any substituents described in [Table L4] to [Table L12](hereinafter, referred to as Compound Class SX₃₄₈₉).

A compound (L-13) wherein R³³ represents a hydrogen atom, T represents a group represented by T2, R¹ represents CF₃, R^{3c} represents a hydrogen atom, and R^{3b} represents any substituents described in [Table L4] to [Table L12](hereinafter, referred to as Compound Class SX₃₄₉₀).

A compound (L-13) wherein R³³ represents a hydrogen atom, T represents a group represented by T2, R¹ represents C₂F5, R^{3c} represents a hydrogen atom, and R^{3b} represents any substituents described in [Table L4] to [Table L12](hereinafter, referred to as Compound Class SX₃₄₉₁).

A compound (L-13) wherein R³³ represents a hydrogen atom, T represents a group represented by T2, R¹ represents OCF₃, R^{3c} represents a hydrogen atom, and R^{3b} represents any substituents described in [Table L4] to [Table L12](hereinafter, referred to as Compound Class SX₃₄₉₂).

A compound (L-13) wherein R³³ represents a hydrogen atom, T represents a group represented by T2, R¹ represents SCF₃, R^{3c} represents a hydrogen atom, and R^{3b} represents any substituents described in [Table L4] to [Table L12](hereinafter, referred to as Compound Class SX₃₄₆₉).

A compound (L-13) wherein R³³ represents a hydrogen atom, T represents a group represented by T2, R¹ represents S(O)CF₃, R^{3c} represents a hydrogen atom, and R^{3b} represents any substituents described in [Table L4] to [Table L12](hereinafter, referred to as Compound Class SX₃₄₉₄).

A compound (L-13) wherein R³³ represents a hydrogen atom, T represents a group represented by T2, R¹ represents S(O)₂CF₃, R^{3c} represents a hydrogen atom, and R^{3b} represents any substituents described in [Table L4] to [Table L12](hereinafter, referred to as Compound Class SX₃₄₉₅).

A compound (L-13) wherein R³³ represents a fluorine atom, T represents a group represented by T2, R¹ represents CF₃, R^{3c} represents a hydrogen atom, and R^{3b} represents any substituents described in [Table L4] to [Table L12](hereinafter, referred to as Compound Class SX₃₄₉₆).

A compound (L-13) wherein R³³ represents a fluorine atom, T represents a group represented by T2, R¹ represents C₂F5, R^{3c} represents a hydrogen atom, and R^{3b} represents any substituents described in [Table L4] to [Table L12](hereinafter, referred to as Compound Class SX₃₄₉₇).

A compound (L-13) wherein R³³ represents a fluorine atom, T represents a group represented by T2, R¹ represents OCF₃, R^{3c} represents a hydrogen atom, and R^{3b} represents any substituents described in [Table L4] to [Table L12](hereinafter, referred to as Compound Class SX₃₄₉₈).

A compound (L-13) wherein R³³ represents a fluorine atom, T represents a group represented by T2, R¹ represents SCF₃, R^{3c} represents a hydrogen atom, and R^{3b} represents any substituents described in [Table L4] to [Table L12](hereinafter, referred to as Compound Class SX₃₄₉₉).

A compound (L-13) wherein R³³ represents a fluorine atom, T represents a group represented by T2, R¹ represents S(O)CF₃, R^{3c} represents a hydrogen atom, and R^{3b} represents any substituents described in [Table L4] to [Table L12](hereinafter, referred to as Compound Class SX₃₅₀₀).

A compound (L-13) wherein R³³ represents a fluorine atom, T represents a group represented by T2, R¹ represents S(O)₂CF₃, R^{3c} represents a hydrogen atom, and R^{3b} represents any substituents described in [Table L4] to [Table L12](hereinafter, referred to as Compound Class SX₃₅₀₁).

A compound (L-13) wherein R³³ represents a chlorine atom, T represents a group represented by T2, R¹ represents CF₃, R^{3c} represents a hydrogen atom, and R^{3b} represents any substituents described in [Table L4] to [Table L12](hereinafter, referred to as Compound Class SX₃₅₀₂).

A compound (L-13) wherein R³³ represents a chlorine atom, T represents a group represented by T2, R¹ represents C₂F5, R^{3c} represents a hydrogen atom, and R^{3b} represents any substituents described in [Table L4] to [Table L12](hereinafter, referred to as Compound Class SX₃₅₀₃).

A compound (L-13) wherein R³³ represents a chlorine atom, T represents a group represented by T2, R¹ represents OCF₃, R^{3c} represents a hydrogen atom, and R^{3b} represents any substituents described in [Table L4] to [Table L12](hereinafter, referred to as Compound Class SX₃₅₀₄).

A compound (L-13) wherein R³³ represents a chlorine atom, T represents a group represented by T2, R¹ represents SCF₃, R^{3c} represents a hydrogen atom, and R^{3b} represents any substituents described in [Table L4] to [Table L12](hereinafter, referred to as Compound Class SX₃₅₀₅).

A compound (L-13) wherein R³³ represents a chlorine atom, T represents a group represented by T2, R¹ represents S(O)CF₃, R^{3c} represents a hydrogen atom, and R^{3b} represents any substituents described in [Table L4] to [Table L12](hereinafter, referred to as Compound Class SX₃₅₀₆).

A compound (L-13) wherein R³³ represents a chlorine atom, T represents a group represented by T2, R¹ represents S(O)₂CF₃, R^{3c} represents a hydrogen atom, and R^{3b} represents any substituents described in [Table L4] to [Table L12](hereinafter, referred to as Compound Class SX₃₅₀₇).

A compound (L-13) wherein R³³ represents a bromine atom, T represents a group represented by T2, R¹ represents CF₃, R^{3c} represents a hydrogen atom, and R^{3b} represents any substituents described in [Table L4] to [Table L12](hereinafter, referred to as Compound Class SX₃₅₀₈).

A compound (L-13) wherein R³³ represents a bromine atom, T represents a group represented by T2, R¹ represents C₂F5, R^{3c} represents a hydrogen atom, and R^{3b} represents any substituents described in [Table L4] to [Table L12](hereinafter, referred to as Compound Class SX₃₅₀₉).

A compound (L-13) wherein R³³ represents a bromine atom, T represents a group represented by T2, R¹ represents OCF₃, R^{3c} represents a hydrogen atom, and R^{3b} represents any substituents described in [Table L4] to [Table L12](hereinafter, referred to as Compound Class SX₃₅₁₀).

A compound (L-13) wherein R³³ represents a bromine atom, T represents a group represented by T2, R¹ represents SCF₃, R^{3c} represents a hydrogen atom, and R^{3b} represents any substituents described in [Table L4] to [Table L12](hereinafter, referred to as Compound Class SX₃₅₁₁).

A compound (L-13) wherein R³³ represents a bromine atom, T represents a group represented by T2, R¹ represents S(O)CF₃, R^{3c} represents a hydrogen atom, and R^{3b} represents any substituents described in [Table L4] to [Table L12](hereinafter, referred to as Compound Class SX₃₅₁₂).

A compound (L-13) wherein R³³ represents a bromine atom, T represents a group represented by T2, R¹ represents S(O)₂CF₃, R^{3c} represents a hydrogen atom, and R^{3b} represents any substituents described in [Table L4] to [Table L12](hereinafter, referred to as Compound Class SX₃₅₁₃).

A compound (L-13) wherein R³³ represents an iodine atom, T represents a group represented by T2, R¹ represents CF₃, R^{3c} represents a hydrogen atom, and R^{3b} represents any substituents described in [Table L4] to [Table L12](hereinafter, referred to as Compound Class SX₃₅₁₄).

A compound (L-13) wherein R³³ represents an iodine atom, T represents a group represented by T2, R¹ represents C₂F5, R^{3c} represents a hydrogen atom, and R^{3b} represents any substituents described in [Table L4] to [Table L12](hereinafter, referred to as Compound Class SX₃₅₁₅).

A compound (L-13) wherein R³³ represents an iodine atom, T represents a group represented by T2, R¹ represents OCF₃, R^{3c} represents a hydrogen atom, and R^{3b} represents any substituents described in [Table L4] to [Table L12](hereinafter, referred to as Compound Class SX₃₅₁₆).

A compound (L-13) wherein R³³ represents an iodine atom, T represents a group represented by T2, R¹ represents SCF₃, R^{3c} represents a hydrogen atom, and R^{3b} represents any substituents described in [Table L4] to [Table L12](hereinafter, referred to as Compound Class SX₃₅₁₇).

A compound (L-13) wherein R³³ represents an iodine atom, T represents a group represented by T2, R¹ represents S(O)CF₃, R^{3c} represents a hydrogen atom, and R^{3b} represents any substituents described in [Table L4] to [Table L12](hereinafter, referred to as Compound Class SX₃₅₁₈).

A compound (L-13) wherein R³³ represents an iodine atom, T represents a group represented by T2, R¹ represents S(O)₂CF₃, R^{3c} represents a hydrogen atom, and R^{3b} represents any substituents described in [Table L4] to [Table L12](hereinafter, referred to as Compound Class SX₃₅₁₉).

A compound (L-13) wherein R³³ represents a hydrogen atom, T represents a group represented by T3, R¹ represents CF₃, R^{3c} represents a hydrogen atom, and R^{3b} represents any substituents described in [Table L4] to [Table L12](hereinafter, referred to as Compound Class SX₃₅₂₀).

A compound (L-13) wherein R³³ represents a hydrogen atom, T represents a group represented by T3, R¹ represents C₂F5, R^{3c} represents a hydrogen atom, and R^{3b} represents any substituents described in [Table L4] to [Table L12](hereinafter, referred to as Compound Class SX₃₅₂₁).

A compound (L-13) wherein R³³ represents a hydrogen atom, T represents a group represented by T3, R¹ represents OCF₃, R^{3c} represents a hydrogen atom, and R^{3b} represents any substituents described in [Table L4] to [Table L12](hereinafter, referred to as Compound Class SX₃₅₂₂).

A compound (L-13) wherein R³³ represents a hydrogen atom, T represents a group represented by T3, R¹ represents SCF₃, R^{3c} represents a hydrogen atom, and R^{3b} represents any substituents described in [Table L4] to [Table L12](hereinafter, referred to as Compound Class SX₃₅₂₃).

A compound (L-13) wherein R³³ represents a hydrogen atom, T represents a group represented by T3, R¹ represents S(O)CF₃, R^{3c} represents a hydrogen atom, and R^{3b} represents any substituents described in [Table L4] to [Table L12](hereinafter, referred to as Compound Class SX₃₅₂₄).

A compound (L-13) wherein R³³ represents a hydrogen atom, T represents a group represented by T3, R¹ represents S(O)₂CF₃, R^{3c} represents a hydrogen atom, and R^{3b} represents any substituents described in [Table L4] to [Table L12](hereinafter, referred to as Compound Class SX₃₅₂₅).

A compound (L-13) wherein R³³ represents a fluorine atom, T represents a group represented by T3, R¹ represents CF₃, R^{3c} represents a hydrogen atom, and R^{3b} represents any substituents described in [Table L4] to [Table L12](hereinafter, referred to as Compound Class SX₃₅₂₆).

A compound (L-13) wherein R³³ represents a fluorine atom, T represents a group represented by T3, R¹ represents C₂F5, R^{3c} represents a hydrogen atom, and R^{3b} represents any substituents described in [Table L4] to [Table L12](hereinafter, referred to as Compound Class SX₃₅₂₇).

A compound (L-13) wherein R³³ represents a fluorine atom, T represents a group represented by T3, R¹ represents OCF₃, R^{3c} represents a hydrogen atom, and R^{3b} represents any substituents described in [Table L4] to [Table L12](hereinafter, referred to as Compound Class SX₃₅₂₈).

A compound (L-13) wherein R³³ represents a fluorine atom, T represents a group represented by T3, R¹ represents SCF₃, R^{3c} represents a hydrogen atom, and R^{3b} represents any substituents described in [Table L4] to [Table L12](hereinafter, referred to as Compound Class SX₃₅₂₉).

A compound (L-13) wherein R³³ represents a fluorine atom, T represents a group represented by T3, R¹ represents S(O)CF₃, R^{3c} represents a hydrogen atom, and R^{3b} represents any substituents described in [Table L4] to [Table L12](hereinafter, referred to as Compound Class SX₃₅₃₀).

A compound (L-13) wherein R³³ represents a fluorine atom, T represents a group represented by T3, R¹ represents S(O)₂CF₃, R^{3c} represents a hydrogen atom, and R^{3b} represents any substituents described in [Table L4] to [Table L12](hereinafter, referred to as Compound Class SX₃₅₃₁).

A compound (L-13) wherein R³³ represents a chlorine atom, T represents a group represented by T3, R¹ represents CF₃, R^{3c} represents a hydrogen atom, and R^{3b} represents any substituents described in [Table L4] to [Table L12](hereinafter, referred to as Compound Class SX₃₅₃₂).

A compound (L-13) wherein R³³ represents a chlorine atom, T represents a group represented by T3, R¹ represents C₂F5, R^{3c} represents a hydrogen atom, and R^{3b} represents any substituents described in [Table L4] to [Table L12](hereinafter, referred to as Compound Class SX₃₅₃₃).

A compound (L-13) wherein R³³ represents a chlorine atom, T represents a group represented by T3, R¹ represents OCF₃, R^{3c} represents a hydrogen atom, and R^{3b} represents any substituents described in [Table L4] to [Table L12](hereinafter, referred to as Compound Class SX₃₅₃₄).

A compound (L-13) wherein R³³ represents a chlorine atom, T represents a group represented by T3, R¹ represents SCF₃, R^{3c} represents a hydrogen atom, and R^{3b} represents any substituents described in [Table L4] to [Table L12](hereinafter, referred to as Compound Class SX₃₅₃₅).

A compound (L-13) wherein R³³ represents a chlorine atom, T represents a group represented by T3, R¹ represents S(O)CF₃, R^{3c} represents a hydrogen atom, and R^{3b} represents any substituents described in [Table L4] to [Table L12](hereinafter, referred to as Compound Class SX₃₅₃₆).

A compound (L-13) wherein R³³ represents a chlorine atom, T represents a group represented by T3, R¹ represents S(O)₂CF₃, R^{3c} represents a hydrogen atom, and R^{3b} represents any substituents described in [Table L4] to [Table L12](hereinafter, referred to as Compound Class SX₃₅₃₇).

A compound (L-13) wherein R³³ represents a bromine atom, T represents a group represented by T3, R¹ represents CF₃, R^{3c} represents a hydrogen atom, and R^{3b} represents any substituents described in [Table L4] to [Table L12](hereinafter, referred to as Compound Class SX₃₅₃₈).

A compound (L-13) wherein R³³ represents a bromine atom, T represents a group represented by T3, R¹ represents C₂F5, R^{3c} represents a hydrogen atom, and R^{3b} represents any substituents described in [Table L4] to [Table L12](hereinafter, referred to as Compound Class SX₃₅₃₉).

A compound (L-13) wherein R³³ represents a bromine atom, T represents a group represented by T3, R¹ represents OCF₃, R^{3c} represents a hydrogen atom, and R^{3b} represents any substituents described in [Table L4] to [Table L12](hereinafter, referred to as Compound Class SX₃₅₄₀).

A compound (L-13) wherein R³³ represents a bromine atom, T represents a group represented by T3, R¹ represents SCF₃, R^{3c} represents a hydrogen atom, and R^{3b} represents any substituents described in [Table L4] to [Table L12](hereinafter, referred to as Compound Class SX₃₅₄₁).

A compound (L-13) wherein R³³ represents a bromine atom, T represents a group represented by T3, R¹ represents S(O)CF₃, R^{3c} represents a hydrogen atom, and R^{3b} represents any substituents described in [Table L4] to [Table L12](hereinafter, referred to as Compound Class SX₃₅₄₂).

A compound (L-13) wherein R³³ represents a bromine atom, T represents a group represented by T3, R¹ represents S(O)₂CF₃, R^{3c} represents a hydrogen atom, and R^{3b} represents any substituents described in [Table L4] to [Table L12](hereinafter, referred to as Compound Class SX₃₅₄₃).

A compound (L-13) wherein R³³ represents an iodine atom, T represents a group represented by T3, R¹ represents CF₃, R^{3c} represents a hydrogen atom, and R^{3b} represents any substituents described in [Table L4] to [Table L12](hereinafter, referred to as Compound Class SX₃₅₄₄).

A compound (L-13) wherein R³³ represents an iodine atom, T represents a group represented by T3, R¹ represents C₂F5, R^{3c} represents a hydrogen atom, and R^{3b} represents any substituents described in [Table L4] to [Table L12](hereinafter, referred to as Compound Class SX₃₅₄₅).

A compound (L-13) wherein R³³ represents an iodine atom, T represents a group represented by T3, R¹ represents OCF₃, R^{3c} represents a hydrogen atom, and R^{3b} represents any substituents described in [Table L4] to [Table L12](hereinafter, referred to as Compound Class SX₃₅₄₆).

A compound (L-13) wherein R³³ represents an iodine atom, T represents a group represented by T3, R¹ represents SCF₃, R^{3c} represents a hydrogen atom, and R^{3b} represents any substituents described in [Table L4] to [Table L12](hereinafter, referred to as Compound Class SX₃₅₄₇).

A compound (L-13) wherein R³³ represents an iodine atom, T represents a group represented by T3, R¹ represents S(O)CF₃, R^{3c} represents a hydrogen atom, and R^{3b} represents any substituents described in [Table L4] to [Table L12](hereinafter, referred to as Compound Class SX₃₅₄₈).

A compound (L-13) wherein R³³ represents an iodine atom, T represents a group represented by T3, R¹ represents S(O)₂CF₃, R^{3c} represents a hydrogen atom, and R^{3b} represents any substituents described in [Table L4] to [Table L12](hereinafter, referred to as Compound Class SX₃₅₄₉).

Next, the Formulation Examples of the present compound X are shown below. The “parts” represents “part by weight”. Further, the present compound S represents the compounds described as the compound groups SX1 to SX2832.

### Formulation Example 1

Into a mixture of 10 parts of any one of the present compounds S, 35 parts of xylene, and 35 parts of DMF, and then 14 parts of polyoxyethylene styryl phenyl ether and 6 parts of calcium dodecylbenzene sulfonate are added, followed by mixing them to obtain each formulation.

### Formulation Example 2

Four (4) parts of sodium lauryl sulfate, 2 parts of calcium lignin sulfonate, 20 parts of wet process silica, and 54 parts of diatomaceous earth are mixed, and further 20 parts of any one of the present compounds S is added thereto, followed by mixing them to obtain each formulation.

### Formulation Example 3

To 2 parts of any one of the present compounds S, 1 part of wet process silica, 2 parts of calcium lignin sulfonate, 30 parts of bentonite, and 65 parts of kaolin clay are added, followed by mixing them. To the mixtures is then added an appropriate amount of water, and the mixtures are further stirred, granulated with a granulator, and forced-air dried to obtain each formulation.

### Formulation Example 4

Into an appropriate amount of acetone, 1 part of any one of the present compounds S is mixed, and then 5 parts of wet process silica, 0.3 parts of isopropyl acid phosphate, and 93.7 parts of kaolin clay are added, following by mixing them with stirring thoroughly and removal of acetone from the mixture by evaporation to obtain each formulation.

### Formulation Example 5

A mixture of 35 parts of polyoxyethylene alkyl ether sulfate ammonium salt and wet process silica (weight ratio of 1:1), 20 parts of any one of the present compounds S, and 45 parts of water are enough mixed to obtain each formulation.

### Formulation Example 6

Ten (10) parts of any one of the present compound S, 18 parts of benzyl alcohol 18 parts and 9 parts of DMSO are mixed, and 6.3 parts of GERONOL (registered trademark) TE250, 2.7 parts of Ethylan (registered trademark) NS-500LQ, and 54 parts of solventnaphtha are added thereto, and then mixed to obtain each formulation.

### Formulation Example 7

Into a mixture of 5 parts of xylene and 5 parts of trichloroethane, 0.1 parts of any one of the present compounds S are mixed, and the resulting mixture is then mixed with 89.9 parts of kerosene to obtain each solution.

### Formulation Example 8

Into 0.5 ml of acetone, 10 mg of any one of the present compounds S is mixed, and the solution is added dropwise to 5 g of a solid feed powder for an animal (solid feed powder for rearing and breeding CE-2, manufactured by CLEA Japan, Inc.), followed by mixing the resulting mixtures uniformly, and then by drying them by evaporation of acetone to obtain each poison bait.

### Formulation Example 9

Into an aerosol can, 0.1 parts of any one of the present compounds S and 49.9 parts of Neothiozole (manufactured by Chuo Kasei Co., Ltd.) are placed. After mounting an aerosol valve, 25 parts of dimethyl ether and 25 parts of LPG are filled, followed by shaking and further mounting an actuator to obtain each oily aerosol.

### Formulation Example 10

A mixture of 0.6 parts of any one of the present compounds S, 0.01 parts of 2,6-di-tert-butyl-4-methylphenol, 5 parts of xylene, 3.39 parts of kerosene, and 1 part of Rheodol (registered trademark) MO-60 (manufactured by Kao Corporation), and 50 parts of distilled water are filled into an aerosol container, and a valve part of the container is attached. Then, 40 parts of LPG is filled therein through the valve under pressure to obtain each aqueous aerosol.

### Formulation Example 11

Zero point one (0.1) g of any one of the present compounds S is mixed into 2 ml of propylene glycol, and the resulting solution is impregnated into a ceramic plate having a size of 4.0 cm×4.0 cm and a thickness of 1.2 cm to obtain each thermal fumigant.

### Formulation Example 12

Five (5) parts of any one of the present compounds S, and 95 parts of ethylene-methyl methacrylate copolymer (the ratio of the methyl methacrylate in the copolymer:10 weight %) are melted and kneaded, and the resulting kneaded product is extruded from an extrusion molding machine to obtain each rod-shaped molded product having a length of 15 cm and a diameter of 3 mm.

### Formulation Example 13

Five (5) parts of any one of the present compounds S, and 95 parts of plasticized polyvinyl chloride resin are melted and kneaded, and the resulting kneaded product is extruded from an extrusion molding machine to obtain each rod-shaped molded product having a length of 15 cm and a diameter of 3 mm.

### Formulation Example 14

One-hundred (100) mg of any one of the present compounds S, 68.75 mg of lactose, 237.5 mg of corn starch, 43.75 mg of microcrystalline cellulose, 18.75 mg of polyvinylpyrrolidone, 28.75 mg of sodium carboxymethyl starch, and 2.5 mg of magnesium stearate are mixed, and the resulting mixtures are compressed to an appropriate size to obtain each tablet.

### Formulation Example 15

Twenty-five (25) mg of any one of the present compounds S, 60 mg of lactose, 25 mg of corn starch, 6 mg of carmellose calcium, and an appropriate amount of 5% aqueous hydroxypropyl methylcellulose solution are mixed, and the resulting mixtures are filled into a hard shell gelatin capsule or a hydroxypropyl methylcellulose capsule to obtain each capsule.

### Formulation Example 16

To 100 mg of any one of the present compounds S, 500 mg of fumaric acid, 2000 mg of sodium chloride, 150 mg of methyl paraben, 50 mg of propyl paraben, 25,000 mg of granulated sugar, 13,000 mg of sorbitol (70% solution), 100 mg of Veegum (registered trademark) K (manufactured by Vanderbilt Co.), 35 mg of a perfume, and 500 mg of a coloring agent, distilled water is added so that a final volume is set to be 100 mL, followed by mixing the mixtures to obtain each suspension for oral administration.

### Formulation Example 17

Into a mixture of 5 parts of an emulsifier, 3 parts of benzyl alcohol and 30 parts of propylene glycol, 5 parts of any one of the present compounds S is mixed, and phosphate buffer is added thereto so that a pH of the solution is set to be 6.0 to 6.5, and water is added as the rest parts to obtain each solution for oral administration.

### Formulation Example 18

To a mixture of 57 parts of fractional distillated palm oil and 3 parts of polysorbate 85, 5 parts of aluminium distearate is added, and heated to disperse it. The resulting mixture is cooled to room temperature, and 25 parts of saccharin is dispersed in an oil vehicle. Ten (10) parts of any one of the present compounds S is divided thereto to obtain each paste for oral administration.

### Formulation Example 19

Five (5) parts of any one of the present compounds S is mixed with 95 parts of limestone filler, followed by a wet-granulation of the resulting mixture to obtain each granule for oral administration.

### Formulation Example 20

Into 80 parts of diethylene glycol monoethyl ether, 5 parts of any one of the present compounds S is mixed, and 15 parts of propylene carbonate is added thereto, and the resulting mixture is mixed to obtain each spot-on solution.

### Formulation Example 21

Into 70 parts of diethylene glycol monoethyl ether, 10 parts of any one of the present compounds S is mixed, and 20 parts of 2-octyldodecanol is added thereto, and the resulting mixture is mixed to obtain each pour-on solution.

### Formulation Example 22

To 0.1 parts of any one of the present compounds S, 40 parts of sodium polyoxyethylene laurylether sulfate (25% aqueous solution), 5 parts of lauramidopropyl betaine, 5 parts of coconut fatty acid monoethanolamide, 0.5 parts of carboxy vinyl polymer, and 49.4 parts of purified water are added, and the resulting mixture is enough mixed to obtain each shampoo formulation.

### Formulation Example 23

Zero point fifteen (0.15) parts of any one of the present compounds S, 95 parts of animal feed, as well as 4.85 parts of a mixture of dibasic calcium phosphate, diatomaceous earth, Aerosil (registered trademark), and carbonate (or chalk) are enough mixed to obtain each premix for animal feed.

### Formulation Example 24

Seven point two (7.2) g of any one of the present compounds S, and 92.8 g of Hosco (registered trademark) S-55 are mixed at 100° C., and the resulting mixture is poured into a suppository mold, followed by performing a cooling solidification to obtain each suppository.

Next, Test Examples are used to show an efficacy of the Present compound X on controlling harmful arthropods.

The following test examples were carried out at 25° C.

### Test Method 1

The test compounds is made to a formulation according to a similar method to that described in the Formulation Example 6, and thereto is added water containing 0.03 v/v % of a spreader to prepare a diluted solution containing a prescribed concentration of the test compound.

Cucumber (Cucumis sativus) seedling (on the developmental stage of the second true leaf) is planted in a container and approximately 30 cotton aphids (Aphis gossypii) (all stages of life) are released onto the leaves of the cucumber. After 1 day, the diluted solutions are sprayed into the seedling in a ratio of 10 mL/seedling. Further, after 5 days, the number of the surviving insects is examined and the controlling value is calculated by the following equation.

$$Controlling value ( \% ) = \{ 1 - ( Cb \times Tai ) / ( Cai \times Tb ) \} \times 100$$

wherein the symbols in the formula represent the following descriptions.
Cb: Number of the test insects in untreated group;
Cai: Number of the surviving insects at the time of the investigation in untreated group;
Tb: Number of the test insects in treated group;
Tai: Number of the surviving insects at the time of the investigation in treated group;
Here the “untreated group” represents a group where the similar treatment procedure to that of the treated group except not using the test compound is done.

### Test Example 1-1

The test was conducted by making the prescribed concentration 500 ppm and using the below-mentioned Present compounds X as a test compound according to the test method 1. As a result of the test, the below-mentioned Present compounds X showed 90% or greater as the controlling value.

Present compound: 1-1, 1-2, 2-1, 2-5, 2-7, 2-11, 2-12, 2-15, 2-18, 2-19, 2-21, 2-25, 2-26, 2-36, 3-1, 3-2, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-38, 4-2, 5-1, 5-2, 6-1, 6-2, 6-3, 6-4, 6-5, 6-6, 7-1, 7-2, 7-3, 7-4, 7-6, 8-1, 8-2, 9, 11, 12-1, 12-2 and 13

### Test Example 1-2

The test was conducted by making the prescribed concentration 200 ppm and using the below-mentioned Present compounds X as a test compound according to the test method 1. As a result of the test, the below-mentioned Present compounds X showed 90% or greater as the controlling value.

Present compound: 1-1, 1-2, 2-1, 2-5, 2-7, 2-11, 2-12, 2-15, 2-18, 2-19, 2-21, 2-25, 2-26, 2-36, 3-1, 3-2, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-38, 4-2, 5-1, 5-2, 6-1, 6-2, 6-3, 6-4, 6-5, 6-6, 7-1, 7-2, 7-3, 7-4, 7-6, 8-1, 8-2, 9, 11, 12-1, 12-2, and 13

### Test Method 2

The test compounds are made to a formulation according to a similar method to that described in the Formulation Example 6, and thereto is added water containing 0.03 v/v % of a spreader to prepare a diluted solution containing a prescribed concentration of the test compound.

Rice (Oryza sativa) seedling (on the developmental stage of the second true leaf) is planted in a container, and the diluted solutions are sprayed into the seedling in a ratio of 10 mL/seedling. Thereafter, 20 3rd instar larvae of brown planthoppers (Nilaparvata lugens) are released onto the rice leaves. After 6 days, the morality is calculated by the following equation.

$$Morality ( \% ) = \{ 1 - the number of the surviving insects / 20 \} \times 100$$

### Test Example 2-1

The test was conducted by making the prescribed concentration 500 ppm and using the below-mentioned Present compounds X as a test compound according to the test method 2. As a result of the test, the below-mentioned Present compound X showed 90% or greater as the controlling value.

Present compound: 7-3

### Test Example 2-2

The test was conducted by making the prescribed concentration 200 ppm and using the below-mentioned Present compounds X as a test compound according to the test method 2. As a result of the test, the below-mentioned Present compound X showed 90% or greater as the controlling value.

Present compound: 7-3

### Test Method 3

The test compounds are made to a formulation according to a similar method to that described in the Formulation Example 6, and thereto is added water containing 0.03 v/v % of a spreader to prepare a diluted solution containing a prescribed concentration of the test compound.

Silverleaf whiteflies (Bemisia tabaci) are released on tomato (Lycopersicon esculentum) seedling that is planted in the container, and then spawn for about 24 hours. The seedling are stored for 8 days, and the larvae of silverleaf whiteflies are hatched from the laid eggs. The diluted solutions are sprayed into the seedling in a ratio of 10 mL/seedling. After 7 days, the number of the surviving insects is examined, and the controlling value is calculated by the following equation.

$$Controlling value ( \% ) = \{ 1 - ( Cb \times Tai ) / ( Cai \times Tb ) \} \times 100$$

wherein the symbols in the formula represent the following descriptions.
Cb: Number of the insects shortly before the treatment in untreated group;
Cai: Number of the surviving insects at the time of the investigation in untreated group;
Tb: Number of the insects shortly before the treatment in treated group;
Tai: Number of the surviving insects at the time of the investigation in treated group;
Here the “untreated group” represents a group where the similar treatment procedure to that of the treated group except not using the test compound is done.

### Test Example 3-1

The test was conducted by making the prescribed concentration 500 ppm and using the below-mentioned Present compounds X as a test compound according to the test method 3. As a result of the test, the below-mentioned Present compound X showed 90% or greater as the controlling value.

Present compound: 3-28 and 3-30

### Test Example 3-2

The test was conducted by making the prescribed concentration 200 ppm and using the below-mentioned Present compounds X as a test compound according to the test method 3. As a result of the test, the below-mentioned Present compound X showed 90% or greater as the controlling value.

Present compound: 3-28 and 3-30

### Test Method 4

Test compounds are made to a formulation according to a similar method to that described in the Formulation example 6, and thereto is added water containing 0.03 v/v % of Shindain (registered trademark) to prepare a diluted solution containing a prescribed concentration of the test compound.

Cabbage (Brassicae oleracea) seedling (on the developmental stage of the second to third true leaf) is planted in a cup, and the diluted solutions are sprayed into the seedling at a ratio of 20 mL/seedling. Thereafter, the stem and leaf thereof is cut out and then is installed into a cup that is covered with filter paper on the bed of the cup. Five (5) diamondback moth (Plutella xylostella) at the second instar larval stage are released into the cup. After 5 days, the number of the surviving insects is counted, and the mortality of insects is calculated by the following equation.

$$Mortality ( \% ) = ( 1 - Number of surviving insects / 5 ) \times 100$$

### Test Example 4-1

The test was conducted by making the prescribed concentration 500 ppm and using the below-mentioned Present compounds X as a test compound according to the test method 4. As a result of the test, the below-mentioned Present compound X showed 80% or greater as the mortality of insects.

Present compound: 1-1, 1-2, 2-1, 2-5, 2-6, 2-11, 2-12, 2-15, 2-18, 2-19, 2-21, 2-25, 2-26, 2-36, 3-1, 3-2, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-38, 4-2, 5-1, 5-2, 6-1, 6-2, 6-3, 6-4, 6-5, 6-6, 7-1, 7-2, 7-3, 7-4, 7-5, 7-6, 8-1, 8-2, 9, 11, 12-1, 12-2, and 13

### Test Example 4-2

The test was conducted by making the prescribed concentration 200 ppm and using the below-mentioned Present compounds X as a test compound according to the test method 4. As a result of the test, the below-mentioned Present compound X showed 80% or greater as the mortality of insects.

Present compound: 1-1, 1-2, 2-1, 2-5, 2-6, 2-11, 2-12, 2-15, 2-18, 2-19, 2-21, 2-25, 2-26, 2-36, 3-1, 3-2, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-38, 4-2, 5-1, 5-2, 6-1, 6-2, 6-3, 6-4, 6-5, 6-6, 7-1, 7-2, 7-3, 7-4, 7-5, 7-6, 8-1, 8-2, 9, 11, 12-1, 12-2, and 13

### Test Method 5

Test compounds are made to a formulation according to a similar method to that described in the Formulation example 6, and thereto is added water containing 0.03 v/v % of Shindain (registered trademark) to prepare a diluted solution containing a prescribed concentration of the test compound.

Cabbage (Brassicae oleracea) seedling (on the developmental stage of the third to fourth true leaf) is planted in a cup, and the diluted solutions are sprayed into the seedling at a ratio of 20 mL/seedling. Thereafter, 10 cotton worm (Spodoptera litura) at the third instar larval stage are released. After 6 days, the number of the surviving insects is counted, and the mortality of insects is calculated by the following equation.

$$Mortality ( \% ) = ( 1 - Number of surviving insects / 10 ) \times 100$$

### Test Example 5-1

The test was conducted by making the prescribed concentration 500 ppm and using the below-mentioned Present compounds X as a test compound according to the test method 5. As a result of the test, the below-mentioned Present compound X showed 80% or greater as the mortality of insects.

Present compound: 1-1, 1-2, 2-1, 2-5, 2-11, 2-12, 2-15, 2-18, 2-19, 2-21, 2-25, 2-26, 2-36, 3-1, 3-2, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 4-2, 5-1, 5-2, 6-1, 6-2, 6-3, 6-4, 6-5, 6-6, 7-1, 7-2, 7-3, 7-4, 7-6, 8-1, 8-2, 9, 11, 12-1, 12-2, and 13.

### Test Example 5-2

The test was conducted by making the prescribed concentration 200 ppm and using the below-mentioned Present compounds X as a test compound according to the test method 5. As a result of the test, the below-mentioned Present compound X showed 80% or greater as the mortality of insects.

Present compound: 1-1, 1-2, 2-1, 2-5, 2-11, 2-12, 2-15, 2-18, 2-19, 2-21, 2-25, 2-26, 2-36, 3-1, 3-2, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 4-2, 5-1, 5-2, 6-1, 6-2, 6-3, 6-4, 6-5, 6-6, 7-1, 7-2, 7-3, 7-4, 7-6, 8-1, 8-2, 9, 11, 12-1, 12-2 and 13.

### Test Method 6

Test compounds are made to a formulation according to a similar method to that described in the Formulation example 5, and thereto is added water to prepare a diluted solution containing a prescribed concentration of the test compound.

A filter paper having a diameter of 5.5 cm in diameter is spread on the bottom of the cup, and then 0.7 ml of the diluted solutions are added dropwise to the filter paper and 30 mg of sucrose is uniformly placed on the filter paper as a bait. Ten (10) housefly (Musca domestica) female adults are released into the cup, and the cup is then covered with the lid. After 24 hours, the number of the dead insects is examined, and the mortality of insects is calculated by the following equation.

$$Mortality ( \% ) = ( Number of dead insects / Number of tested insects ) \times 100$$

### Test Example 6

The test was conducted by making the prescribed concentration 500 ppm and using the below-mentioned Present compounds X as a test compound according to the test method 6. As a result of the test, the below-mentioned Present compound X showed 70% or greater as the mortality of insects.

Present compound: 2-2, 2-6, 2-11, 2-12, 2-15, 3-1, 3-2, 3-6, 3-12, 3-13, 3-20, 3-24, 6-1

### Test Method 7

An acetone solution which is adjusted to 800 ppm of the test compound is poured into a container having 20 mL contents, and the test compound is coated uniformly on inner face of the container so as to make 40 mg/m² thereof, and the container is then allowed to dry.

Into the container, five (5) housefly (Musca domestica) female adults are released into the container, and the container is then covered with the lid. After the prescribed time is elapsed, the state of the housefly is examined, and the mortality of insects is calculated by the following equation.

$$Mortality ( \% ) = ( Number of dead insects / Number of tested insects ) \times 100$$

### Test Example 7-1

The test was conducted by making the prescribed elapsed time 1 hour and using the below-mentioned Present compounds X as a test compound according to the test method 7. As a result of the test, the below-mentioned Present compound X showed 80% or greater as the mortality of insects.

Present compound: 2-5, 3-5, 4-2, 6-2, 6-3, 8-1, 8-2

### Test Example 7-2

The test is conducted by making the prescribed elapsed time 1 day and using the below-mentioned Present compounds X as a test compound according to the test method 7, and the effect of the test is confirmed.

### Test Method 8

Test compounds are made to a formulation according to a similar method to that described in the Formulation example 5, and thereto is added water to prepare a diluted solution containing a prescribed concentration of the test compound.

Into the diluted solution, 30 common house mosquito (Culex pipiens pallens) at the last instar larval stage are released, and after 1 day, the state of the house mosquito larvae is examined, and the mortality of insects is calculated by the following equation.

$$Mortality ( \% ) = ( Number of dead insects / Number of tested insects ) \times 100$$

### Test Example 8

The test was conducted by making the prescribed concentration 3.5 ppm and using the below-mentioned Present compounds X as a test compound according to the test method 8. As a result of the test, the below-mentioned Present compound X showed 91% or greater as the mortality of insects.

Present compound: 1-2, 2-1, 2-2, 2-3, 2-5, 2-6, 2-11, 2-12, 2-15, 3-1, 3-2, 3-3, 3-5, 3-6, 3-8, 3-12, 3-13, 3-16, 3-17, 3-20, 3-24, 3-26, 4-2, 5-1, 5-2, 6-1, 6-2, 6-3, 6-5, 6-6, 8-1, 8-2, and 11

### Test Method 9

Each 1 mg of the test compounds is dissolved into 50 μL of a mixed solution of polyoxyethylene sorbitan mono-cocoate and acetone (polyoxyethylene sorbitan mono-cocoate:acetone=5:95 (v/v ratio)). Thereto is added water containing 0.03% by volume of Shindain (registered trademark) to prepare a diluted solution containing a prescribed concentration of the test compound.

A young entire seedling of Corns (Zea mays) is immersed into the diluted solution for 30 seconds. Thereafter, each two grains of the seedlings are installed in a plastic petri dish (90 mm radius), and 10 Western corn rootworms (Diabrotica virgifera virgifera) at the second instar larval stage are released into the dish. After 5 days, the number of the dead insects is counted, and the mortality of insects is calculated by the following equation.

$$Mortality ( \% ) = ( Number of dead insects / 10 \} \} \times 100$$

### Test Example 9-1

The test was conducted by making the prescribed concentration 500 ppm and using the below-mentioned Present compounds X as a test compound according to the test method 9. As a result of the test, the below-mentioned Present compound X showed 80% or greater as the mortality of insects.

Present compound X: 1-2, 2-1, 2-11, 2-18, 2-19, 2-21, 2-25, 2-26, 3-1, 3-9, 3-10, 3-11, 3-12, 3-13, 3-15, 3-16, 3-19, 3-20, 3-21, 3-22, 3-23, 3-24, 3-25, 3-27, 3-28, 3-29, 3-30, 4-2, 5-2, 6-1, 6-2, 6-3, 6-4, 6-5, 6-6, 7-2, 7-6, 8-1, 11, 12-1, and 13

### Test Example 9-2

The test is conducted by making the prescribed concentration 200 ppm and using the below-mentioned Present compounds X as a test compound according to the test method 9, and the effect of the test is confirmed.

### Test Method 10

An acetone solution which is adjusted to 800 ppm of the test compound is poured into a container having 50 mL contents, and the test compound is coated uniformly on inner face of the container so as to make 40 mg/m² thereof, and the container is then allowed to dry.

Into the container, 5 German cockroach (Blattella germanica) male adults are released into the container, and the container is then covered with the lid. After the prescribed time is elapsed, the state of the German cockroach is examined, and the mortality of insects is calculated by the following equation.

$$Mortality ( \% ) = ( Number of dead insects / Number of tested insects ) \times 100$$

### Test Example 10-1

The test is conducted by making the prescribed elapsed time 1 hour and using the below-mentioned Present compounds X as a test compound according to the test method 10, and the effect of the test is confirmed.

### Test Example 10-2

The test is conducted by making the prescribed elapsed time 1 day and using the below-mentioned Present compounds X as a test compound according to the test method 10, and the effect of the test is confirmed.

### Test Example 10-3

The test is conducted by making the prescribed elapsed time 3 days and using the below-mentioned Present compounds X as a test compound according to the test method 10, and the effect of the test is confirmed.

### Test Method 11

An acetone solution which is adjusted to 50 ppm of the test compound is poured into a container having 20 mL contents, and the test compound is coated uniformly on inner face of the container so as to make 2.5 mg/m² thereof, and the container is then allowed to dry.

Into the container, 5 nymph Haemaphysalis longicornis are released into the container, and the container is then covered with the lid. After the prescribed time is elapsed, the state of the Haemaphysalis longicornis is examined, and the mortality of insects is calculated by the following equation.

$$Mortality ( \% ) = ( Number of dead insects / Number of tested insects ) \times 100$$

### Test Example 11

The test is conducted by making the prescribed elapsed time 2 days and using the below-mentioned Present compounds X as a test compound according to the test method 11, and the effect of the test is confirmed.

### Test Method 12

An acetone solution which is adjusted to 50 ppm of the test compound is poured into a container having 20 mL contents, and the test compound is coated uniformly on inner face of the container so as to make 2.5 mg/m² thereof, and the container is then allowed to dry.

Into the container, 10 worked ants of Monomorium pharaonis are released into the container, and the container is then covered with the lid. After the prescribed time is elapsed, the state of the Monomorium pharaonis is examined, and the mortality of insects is calculated by the following equation.

$$Mortality ( \% ) = ( Number of dead insects / Number of tested insects ) \times 100$$

### Test Example 12

The test is conducted by making the prescribed elapsed time 1 hour and using the below-mentioned Present compounds X as a test compound according to the test method 12, and the effect of the test is confirmed.

### Test Method 13

Each 1 mg of the present compound X is dissolved into 10 μL of a mixed solution of xylene, DMF, and a surfactant (xylene:DMF:surfactant=4:4:1 (v/v ratio)). Thereto is added water containing 0.02% by volume of a spreader to prepare diluted solution A containing a prescribed concentration of the present compound.

Each 1 mg of the present ingredients is dissolved into 10 μL of a mixed solution of xylene, DMF, and a surfactant (xylene:DMF:surfactant=4:4:1 (v/v ratio)). Thereto is added water containing 0.02% by volume of a spreader to prepare diluted solution B containing a prescribed concentration of the present ingredient.

The diluted solution A is mixed with the diluted solution B to prepare diluted solution C.

Leaf discs of Cucumber (Cucumber sativus) cotyledon (length 1.5 cm) are placed in each well of 24-well microplate. Two (2) apterous adults and 8 larvae of cotton aphids (Aphis gossypii) per one well are released and the diluted solution C is sprayed at 20 μL per one well. The group is defined as “treated group”. A well that is sprayed with 20 μL of water containing 0.02% by volume of a spreader instead of the diluted solution C is defined as “untreated group”.

After drying the diluted solution C, the upper microplate is covered with a film sheet. After 5 days, the number of the surviving insects in each well is examined.

The controlling value is calculated by the following equation.

$$Controlling value ( \% ) = \{ 1 - ( Tai ) / ( Cai ) \} \times 100$$

wherein the symbols in the equation represent the following descriptions.
Cai: Number of the surviving insects at the time of the examination in untreated group;
Tai: Number of the surviving insects at the time of the examination in treated group.

Specific diluted solutions C, which can confirm their effect according to the Test method 13, are described in the following 1) to 5).

1) The diluted solution C comprises the combination recited in List A wherein a concentration of the present compound X is 200 ppm and a concentration of the present ingredient is 2000 ppm. In List A, Comp λ represents any one compound selected from the present compound 1-1, 1-2, 2-1, 2-2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 3-1, 3-2, 3-3, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 4-1, 4-2, 5-1, 5-2, 6-1, 6-2, 6-3, 6-4, 6-5, 6-6, 7-1, 7-2, 7-3, 7-4, 7-5, 7-6, 8-1, 8-2, 9, 10-1, 11, 12-1, 12-2, and 13.

### List A:

Comp X+Clothianidin; Comp X+thiamethoxam; Comp X+imidacloprid; Comp X+thiacloprid; Comp X+flupyradifurone; Comp X+sulfoxaflor; Comp X+triflumezopyrim; Comp X+dicloromezotiaz; Comp X+beta-cyfluthrin; Comp X+tefluthrin; Comp X+fipronil; Comp X+chlorantraniliprole; Comp X+cyantraniliprole; Comp X+tetraniliprole; Comp X+thiodicarb; Comp X+carbofuran; Comp X+fluxametamide; Comp X+afoxolaner; Comp X+fluralaner; Comp X+broflanilide; Comp X+abamectin; Comp X+fluopyram; Comp X+fluensulfone; Comp X+fluazaindolizine; Comp X+tioxazafen; Comp X+flupyrimin; Comp X+Mycorrhizal Fungi; Comp X+Bradyrhizobium japonicum TA-11; Comp X+Bacillus firmus; Comp X+Bacillus firmus I-1582; Comp X+Bacillus amyloliquefaciens; Comp X+Bacillus amyloliquefaciens FZB42; Comp X+Pasteuria nishizawae; Comp X+Pasteuria nishizawae Pnl; Comp X+Pasteuria penetrans; Comp X+tebuconazole; Comp X+prothioconazole; Comp X+metconazole; Comp X+ipconazole; Comp X+triticonazole; Comp X+difenoconazole; Comp X+imazalil; Comp X+triadimenol; Comp X+tetraconazole; Comp X+flutriafol; Comp X+mandestrobin; Comp X+azoxystrobin; Comp X+pyraclostrobin; Comp X+trifloxystrobin; Comp X+fluoxastrobin; Comp X+picoxystrobin; Comp X+fenamidone; Comp X+metalaxyl; Comp X+metalaxyl-M; Comp X+fludioxonil; Comp X+sedaxane; Comp X+penflufen; Comp X+fluxapyroxad; Comp X+benzovindiflupyr; Comp X+boscalid; Comp X+carboxin; Comp X+penthiopyrad; Comp X+flutolanil; Comp X+captan; Comp X+thiram; Comp X+tolclofos-methyl; Comp X+thiabendazole; Comp X+ethaboxam; Comp X+mancozeb; Comp X+picarbutrazox; Comp X+oxathiapiprolin; Comp X+silthiofam; Comp X+inpyrfluxam.

2) The diluted solution C comprises the combination recited in List A wherein a concentration of the present compound is 200 ppm, and a concentration of the present ingredient is 200 ppm.
3) The diluted solution C comprises the combination recited in List A wherein a concentration of the present compound X is 500 ppm, and a concentration of the present ingredient is 50 ppm.
4) The diluted solution C comprises the combination recited in List A wherein a concentration of the present compound X is 500 ppm, and a concentration of the present ingredient is 5 ppm.
5) The diluted solution C comprises the combination recited in List A wherein a concentration of the present compound X is 500 ppm, and a concentration of the present ingredient is 0.5 ppm.

### INDUSTRIAL APPLICABILITY

The present compound X (including the present compound) shows an excellent control effect against a harmful arthropod.

The invention claimed is:

1. A compound represented by formula (I): 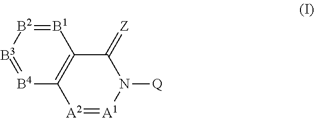
wherein
Q represents a group represented by formula Q1, or a group represented by formula Q2, 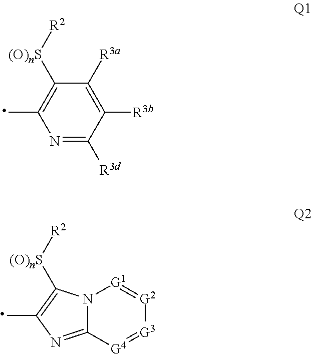
Z represents an oxygen atom or a sulfur atom,
A¹ represents CR^{4a}, and A² represents a nitrogen atom or CR^{4b},
a combination of B¹, B², B³ and B⁴ represents,
a combination in which B¹ represents a nitrogen atom or CR^{6a}, B² represents CR¹, B³ represents a nitrogen atom or CR^{6c}, and B⁴ represents a nitrogen atom or CR^{6d};
a combination in which B¹ represents a nitrogen atom or CR^{6a}, B² represents a nitrogen atom or CR^{6b}, B³ represents CR¹, and B⁴ represents a nitrogen atom or CR^{6d};
a combination in which B¹ represents a nitrogen atom or CR^{6a}, B² represents a nitrogen atom or CR^{6b}, B³ represents CR^{6c}, and B⁴ represents CR¹;
a combination in which B¹ represents a nitrogen atom or CR^{6a}, B² represents CR^{6b}, B³ represents a nitrogen atom, and B⁴ represents CR¹; or
a combination in which B¹ represents CR^{6a}, B² represents a nitrogen atom, B³ represents a nitrogen atom, and B⁴ represents CR¹,
R¹ represents a C1-C6 chain hydrocarbon group having one or more substituents selected from the group consisting of cyano group and halogen atom, a C3-C4 cycloalkyl group optionally having one or more substituents selected from the group consisting of cyano group and halogen atom, S(O)ₘR⁸, OR⁸, halogen atom or OS(O)₂R⁸,
m represents 0, 1 or 2,
R⁸ represents a C1-C6 chain hydrocarbon group having one or more substituents selected from the group consisting of cyano group and halogen atom; or a C3-C4 cycloalkyl group optionally having one or more substituents selected from the group consisting of cyano group and halogen atom,
R^{4a}, R^{4b}, R^{6a}, R^{6b}, R^{6c} and R^{6d} are identical to or different from each other and each represents a C1-C6 chain hydrocarbon group optionally having one or more halogen atoms, a C3-C7 cycloalkyl group optionally having one or more halogen atoms, a C1-C6 alkoxy group optionally having one or more halogen atoms, NR⁹R¹⁰, C(O)R⁷, C(O)NR¹⁹R²⁰, NR⁹C(O)R¹⁸, NR⁹C(O)OR¹⁸, NR⁹C(O)NR¹⁹R²⁰, cyano group, halogen atom or a hydrogen atom,
R⁹ and R¹⁹ are identical to or different from each other and each represents a C1-C6 chain hydrocarbon group optionally having one or more halogen atoms, or a hydrogen atom,
R¹⁰ represents a C1-C6 chain hydrocarbon group optionally having one or more substituents selected from Group F, a C3-C7 cycloalkyl group optionally having one or more substituents selected from Group J, a C3-C7 cycloalkenyl group optionally having one or more substituents selected from Group J, a phenyl group optionally having one or more substituents selected from Group D, a six (6) membered aromatic heterocyclic group optionally having one or more substituents selected from Group D, a hydrogen atom, or S(O)₂R²¹,
R²¹ represents a C1-C6 chain hydrocarbon group optionally having one or more halogen atoms, a C3-C7 cycloalkyl group optionally having one or more halogen atoms, or a phenyl group optionally having one or more substituents selected from Group D,
R⁷, R¹⁸, and R²⁰ are identical to or different from each other and each represents a C1-C6 chain hydrocarbon group optionally having one or more substituents selected from Group F, a C3-C7 cycloalkyl group optionally having one or more substituents selected from Group J, or a hydrogen atom,
n represents 0, 1 or 2,
R² represents a C1-C6 chain hydrocarbon group optionally having one or more halogen atoms, a cyclopropyl group, or a cyclopropylmethyl group,
G¹ represents a nitrogen atom or CR^{3a},
G² represents CR^{3b},
G³ represents a nitrogen atom or CR^{3c},
G⁴ represents a nitrogen atom or CR^{3d},
R^{3a}, R^{3c} and R^{3d} are identical to or different from each other and each represents a C1-C6 chain hydrocarbon group optionally having one or more substituents selected from Group B, a C3-C7 cycloalkyl group optionally having one or more substituents selected from Group E, a phenyl group optionally having one or more substituents selected from Group H, 5 or 6 membered aromatic heterocyclic group optionally having one or more substituents selected from Group H, OR¹², NR¹¹R¹², NR^{11a}R^{12a}, NR²⁴NR¹¹R¹², N²⁴OR¹¹, NR¹¹C(O)R¹³, NR²⁴NR¹¹C(O)R¹³, NR¹¹C(O)OR¹⁴, NR²⁴NR¹¹C(O)OR¹⁴, NR¹¹C(O)NR³¹R³², NR²⁴NR¹¹C(O)NR³¹R³², N═CHNR³¹R³², N═S(O)ₚR¹⁵R¹⁶, C(O)R¹³, C(O)OR¹⁷, C(O)NR³¹R³², C(O)NR¹¹S(O)₂R²³, CR^{3c}═NOR¹⁷, NR¹¹CR²⁴═NOR¹⁷, S(O)_{q}R²³, a cyano group, a nitro group, a hydrogen atom, or a halogen atom,
a combination of A² and R^{3b} represents,
a combination in which A² represents a nitrogen atom, R^{3b} represents a C1-C6 chain hydrocarbon group optionally having one or more substituents selected from Group B, a C3-C7 cycloalkyl group optionally having one or more substituents selected from Group E, a phenyl group optionally having one or more substituents selected from Group H, a 5 or 6 membered aromatic heterocyclic group optionally having one or more substituents selected from Group H, OR¹², NR¹¹R¹², NR^{11a}R^{12a}, NR²⁴NR¹¹R¹², NR²⁴OR¹¹, NR¹¹C(O)R¹³, NR²⁴NR¹¹C(O)R¹³, NR¹¹C(O)OR¹⁴, NR²⁴NR¹¹C(O)OR¹⁴, NR¹¹C(O)NR³¹R³², NR²⁴NR¹¹C(O)NR³¹R³², N═CHNR³¹R³², N═S(O)ₚR¹⁵R¹⁶, C(O)R¹³, C(O)OR¹⁷, C(O)NR³¹R³², C(O)NR¹¹S(O)₂R²³, CR^{3c}═NOR¹⁷, NR¹¹CR²⁴═NOR¹⁷, S(O)_{q}R²³, a cyano group, a nitro group, or a hydrogen atom; or
a combination in which A² represents CR^{4b}, R^{3b} represents a C1-C6 chain hydrocarbon group optionally having one or more substituents selected from Group B, a C3-C7 cycloalkyl group optionally having one or more substituents selected from Group E, a phenyl group optionally having one or more substituents selected from Group H, a 5 or 6 membered aromatic heterocyclic group optionally having one or more substituents selected from Group H, OR¹², NR¹¹R¹², NR^{11a}R^{12a}, NR²⁴NR¹¹R¹², NR²⁴OR¹¹, NR¹¹C(O)R¹³, NR²⁴NR¹¹C(O)R¹³, NR¹¹C(O)OR¹⁴, NR²⁴NR¹¹C(O)OR¹⁴, NR¹¹C(O)NR³¹R³², NR²⁴NR¹¹C(O)NR³¹R³², N═CHNR³¹R³², N═S(O)ₚR¹⁵R¹⁶, C(O)R¹³, C(O)OR¹⁷, C(O)NR³¹R³², C(O)NR¹¹S(O)₂R²³, CR^{3c}═NOR¹⁷, NR¹¹CR²⁴═NOR¹⁷, S(O)_{q}R²³, a cyano group, a nitro group, a hydrogen atom, or a halogen atom,
p represents 0 or 1,
q represents 0 or 1,
R^{3c} represents a C1-C6 chain hydrocarbon group optionally having one or more halogen atoms, a halogen atom OR³⁵, NR³⁶R³⁷, or a hydrogen atom,
R³⁵ represents a C1-C6 chain hydrocarbon group optionally having one or more halogen atoms,
R¹⁷ represents a C1-C6 chain hydrocarbon group optionally having one or more halogen atoms, a phenyl group optionally having one or more substituents selected from Group D, or a hydrogen atom,
R¹¹, R²⁴, R³⁶ and R³⁷ are identical to or different from each other and each represents a C1-C6 chain hydrocarbon group optionally having one or more halogen atoms, or a hydrogen atom,
R¹² represents a C1-C6 chain hydrocarbon group optionally having one or more substituents selected from Group F, a C3-C7 cycloalkyl group optionally having one or more substituents selected from Group J, a C3-C7 cycloalkenyl group optionally having one or more substituents selected from Group J, a phenyl group optionally having one or more substituents selected from Group D, a 6 membered aromatic heterocyclic group optionally having one or more substituents selected from Group D, a hydrogen atom, or S(O)₂R²³,
R²³ represents a C1-C6 chain hydrocarbon group optionally having one or more halogen atoms, or a phenyl group optionally having one or more substituents selected from Group D,
R^{11a} and R^{12a} combined together with a nitrogen atom to which they are attached represent 3 to 7 membered nonaromatic heterocyclic group optionally having one or more substituents selected from Group E,
R¹³ represents a C1-C6 chain hydrocarbon group optionally having one or more halogen atoms, a C3-C7 cycloalkyl group optionally having one or more halogen atoms, a (C3-C6 cycloalkyl)C1-C3 alkyl group optionally having one or more halogen atoms, a phenyl group optionally having one or more substituents selected from Group D, a 5 or 6 membered aromatic heterocyclic group optionally having one or more substituents selected from Group D, or a hydrogen atom,
R¹⁴ represents a C1-C6 chain hydrocarbon group optionally having one or more halogen atoms, a C3-C7 cycloalkyl group optionally having one or more halogen atoms, a (C3-C6 cycloalkyl)C1-C3 alkyl group optionally having one or more halogen atoms, or a phenyl C1-C3 alkyl group {the phenyl moiety in the phenyl C1-C3 alkyl group may have optionally one or more substituents selected from Group D},
R¹⁵ and R¹⁶ are identical to or different from each other and each represents a C1-C6 alkyl group optionally having one or more halogen atoms,
R³¹ represents a C1-C6 alkyl group optionally having one or more halogen atoms, or a hydrogen atom,
R³² represents a C1-C6 chain hydrocarbon group optionally having one or more substituents selected from Group F, a C3-C7 cycloalkyl group optionally having one or more substituents selected from Group J, or a hydrogen atom,
when Q represents a group represented by formula Q1, R^{3b} and R^{3d} combined together with two carbon atoms to which they are attached may form a benzene ring, a pyrrole ring, a furan ring, a thiophene ring, a pyrazole ring, an imidazole ring, an oxazole ring, an isoxazole ring, a thiazole ring, an isothiazole ring, an oxadiazole ring, a thiadiazole ring, a pyridine ring, a pyridazine ring, a pyrimidine ring, a pyrazine ring {the benzene ring, the pyrrole ring, the furan ring, the thiophene ring, the pyrazole ring, the imidazole ring, the oxazole ring, the isoxazole ring, the thiazole ring, the isothiazole ring, the pyridine ring, the pyridazine ring, the pyrimidine ring, and the pyrazine ring each may have optionally one or more substituents selected from Group H}, or a triazole ring optionally having one or more substituents selected from Group I,
Group B: a group consisting of a C1-C6 alkoxy group optionally having one or more halogen atoms, a C3-C6 alkenyloxy group optionally having one or more halogen atoms, a C3-C6 alkynyloxy group optionally having one or more halogen atoms, a C1-C6 alkylsulfanyl group optionally having one or more halogen atoms, a C1-C6 alkylsulfinyl group optionally having one or more halogen atoms, a C1-C6 alkylsulfonyl group optionally having one or more halogen atoms, a C3-C6 cycloalkyl group optionally having one or more halogen atoms, a C1-C6 alkylamino group optionally having one or more halogen atoms, a di(C1-C4 alkyl)amino group optionally having one or more halogen atoms, a C2-C6 alkylcarbonyl group optionally having one or more halogen atoms, a C2-C6 alkoxycarbonyl group optionally having one or more halogen atoms, a C2-C6 alkoxycarbonyloxy group optionally having one or more halogen atoms, an aminocarbonyl group, a C1-C6 alkylaminocarbonyl group optionally having one or more halogen atoms, a di(C1-C4 alkyl)aminocarbonyl group optionally having one or more halogen atoms, a C2-C6 alkoxycarbonylamino group optionally having one or more halogen atoms, a (C2-C6 alkoxycarbonyl)(C1-C6 alkyl) amino group optionally having one or more halogen atoms, a cyano group, an amino group, a nitro group, a hydroxy group, and a halogen atom,
Group D: a group consisting of a C1-C6 chain hydrocarbon group optionally having one or more halogen atoms, a C1-C6 alkoxy group optionally having one or more halogen atoms, a C3-C6 alkenyloxy group optionally having one or more halogen atoms, a C3-C6 alkynyloxy group optionally having one or more halogen atoms, a C1-C6 alkylsulfanyl group optionally having one or more halogen atoms, a C1-C6 alkylsulfinyl group optionally having one or more halogen atoms, a C1-C6 alkylsulfonyl group optionally having one or more halogen atoms, a C3-C6 cycloalkyl group optionally having one or more halogen atoms, a C1-C6 alkylamino group optionally having one or more halogen atoms, a di(C1-C4 alkyl)amino group optionally having one or more halogen atoms, a C2-C6 alkylcarbonyl group optionally having one or more halogen atoms, a C2-C6 alkoxycarbonyl group optionally having one or more halogen atoms, a C2-C6 alkoxycarbonyloxy group optionally having one or more halogen atoms, an aminocarbonyl group, a C1-C6 alkylaminocarbonyl group optionally having one or more halogen atoms, a di(C1-C4 alkyl)aminocarbonyl group optionally having one or more halogen atoms, a C2-C6 alkoxycarbonylamino group optionally having one or more halogen atoms, a (C2-C6 alkoxycarbonyl)(C1-C6 alkyl) amino group optionally having one or more halogen atoms, a cyano group, an amino group, a nitro group, a hydroxy group, and a halogen atom,
Group E: a group consisting of a C1-C6 chain hydrocarbon group optionally having one or more halogen atoms, a C1-C6 alkoxy group optionally having one or more halogen atoms, a C3-C6 alkenyloxy group optionally having one or more halogen atoms, a C3-C6 alkynyloxy group optionally having one or more halogen atoms, a C1-C6 alkylamino group optionally having one or more halogen atoms, a di(C1-C4 alkyl)amino group optionally having one or more halogen atoms, a C2-C6 alkylcarbonyl group optionally having one or more halogen atoms, a C2-C6 alkoxycarbonyl group optionally having one or more halogen atoms, a C2-C6 alkoxycarbonyloxy group optionally having one or more halogen atoms, an aminocarbonyl group, a C1-C6 alkylaminocarbonyl group optionally having one or more halogen atoms, a di(C1-C4 alkyl)aminocarbonyl group optionally having one or more halogen atoms, a C2-C6 alkoxycarbonylamino group optionally having one or more halogen atoms, a (C2-C6 alkoxycarbonyl)(C1-C6 alkyl) amino group optionally having one or more halogen atoms, a cyano group, an amino group, a nitro group, a hydroxy group, an oxo group, and a halogen atom,
Group F: a group consisting of a C3-C7 cycloalkyl group optionally having one or more halogen atoms, a phenyl group optionally having one or more substituents selected from Group D, a 5 or 6 membered aromatic heterocyclic group optionally having one or more substituents selected from Group D, a C1-C6 alkoxy group optionally having one or more halogen atoms, a C1-C6 alkylamino group optionally having one or more halogen atoms, a di(C1-C4 alkyl)amino group optionally having one or more halogen atoms, a cyano group, an amino group, a nitro group, a hydroxy group, and a halogen atom,
Group H: a group consisting of a C1-C6 chain hydrocarbon group optionally having one or more halogen atoms, a C3-C6 cycloalkyl group optionally having one or more halogen atoms, a phenyl group optionally having one or more substituents selected from Group D, a 5 or 6 membered aromatic heterocyclic group optionally having one or more substituents selected from Group D, a C1-C6 alkoxy group optionally having one or more halogen atoms, a C1-C6 alkylamino group optionally having one or more halogen atoms, a di(C1-C4 alkyl)amino group optionally having one or more halogen atoms, a C2-C6 alkylcarbonyl group optionally having one or more halogen atoms, a C2-C6 alkoxycarbonyl group optionally having one or more halogen atoms, a C2-C6 alkoxycarbonyloxy group optionally having one or more halogen atoms, an aminocarbonyl group, a (C1-C6 alkyl)aminocarbonyl group optionally having one or more halogen atoms, a di(C1-C4 alkyl)aminocarbonyl group optionally having one or more halogen atoms, a C2-C6 alkoxycarbonylamino group optionally having one or more halogen atoms, a (C2-C6 alkoxycarbonyl)(C1-C6 alkyl) amino group optionally having one or more halogen atoms, a cyano group, an amino group, a nitro group, a hydroxy group, and a halogen atom,
Group I: a group consisting of a C2-C6 chain hydrocarbon group optionally having one or more halogen atoms, a C3-C6 cycloalkyl group optionally having one or more halogen atoms, a phenyl group optionally having one or more substituents selected from Group D, a 5 or 6 membered aromatic heterocyclic group optionally having one or more substituents selected from Group D, a C2-C6 alkylcarbonyl group optionally having one or more halogen atoms, a C2-C6 alkoxycarbonyl group optionally having one or more halogen atoms, an aminocarbonyl group, a (C1-C6 alkyl)aminocarbonyl group optionally having one or more halogen atoms, and a di(C1-C4 alkyl)aminocarbonyl group optionally having one or more halogen atoms,
Group J: a group consisting of a C1-C6 alkyl group optionally having one or more halogen atoms, a C1-C6 alkoxy group optionally having one or more halogen atoms, a C2-C6 alkoxycarbonyl group optionally having one or more halogen atoms, an amino group, a cyano group, and a halogen atom,
or N-oxide thereof.

2. The compound according to claim 1 or N-oxide thereof, wherein
R¹ represents a C1-C6 chain hydrocarbon group having one or more substituents selected from the group consisting of cyano group and halogen atom, a C3-C4 cycloalkyl group optionally having one or more substituents selected from the group consisting of cyano group and halogen atom, S(O)ₘR⁸, OR⁸, or OS(O)₂R⁸.

3. The compound according to claim 1 or N-oxide thereof, wherein
R^{3a} represents a hydrogen atom, R^{3c} and R^{3d} are identical to or different from each other and each represents a C1-C6 alkyl group, a C2-C6 alkenyl group, a C3-C7 cycloalkyl group {the C1-C6 alkyl group, the C2-C6 alkenyl group, and the C3-C7 cycloalkyl group each may have optionally one or more substituents selected from the group consisting of halogen atom and cyano group}, a phenyl group, a triazolyl group, a pyridyl group, a pyrimidinyl group {the phenyl group, the triazolyl group, the pyridyl group, and the pyrimidinyl group each may have optionally one or more substituents selected from Group J}, OR¹², CR^{3c}═NOR¹⁷, a hydrogen atom, or a halogen atom, and when Q represents a group represented by formula Q1, and R^{3b} and R^{3d} combined together with two carbon atoms to which they are attached may form a benzene ring {the benzene ring may have optionally one or more substituents selected from a group consisting of C1-C6 alkyl group optionally having one or more halogen atoms, and a halogen atom}.

4. The compound according to claim 1 or N-oxide thereof, wherein
R^{3a} and R^{3d} represent a hydrogen atom, and R^{3c} represents a C1-C6 alkyl group optionally having one or more halogen atoms, a cyclopropyl group, a hydrogen atom, or a halogen atom, and when Q represents a group represented by formula Q1, R^{3b} and R^{3d} combined together with two carbon atoms to which they are attached may form a benzene ring {the benzene ring may have optionally one or more substituents selected from a group consisting of C1-C6 alkyl group optionally having one or more halogen atoms, and halogen atom}.

5. The compound according to claim 1 or N-oxide thereof, wherein Q represents a group represented by formula Q1.

6. The compound according to claim 1 or N-oxide thereof, wherein Q represents a group represented by formula Q1, and R^{3a} and R^{3d} represent a hydrogen atom.

7. The compound according to claim 1 or N-oxide thereof, wherein Q represents a group represented by formula Q2.

8. The compound according to claim 1 or N-oxide thereof, wherein B¹ represents CH, a combination of B², B³ and B⁴ represents a combination in which B² represents CR¹, B³ represents CR^{6c}, and B⁴ represents CR^{6d}; a combination in which B² represents CR^{6b}, B³ represents CR¹, and B⁴ represents CR^{6d};
or a combination in which B² represents CR^{6b}, B³ represents CR^{6c}, and B⁴ represents CR¹.

9. The compound according to claim 1 or N-oxide thereof, wherein G¹ represents a nitrogen atom or CH, G³ represents CR^{3c}, and G⁴ represents a nitrogen atom or CH.

10. The compound according to claim 1 or N-oxide thereof, wherein G¹ represents CH, G³ represents CR^{3c}, and G⁴ represents CH.

11. The compound according to claim 1 or N-oxide thereof, wherein R¹ represents a C1-C6 alkyl group having one or more substituents selected from a group consisting of halogen atom and cyano group; a cyclopropyl group optionally having one or more substituents selected from the group consisting of cyano group and halogen atom; S(O)ₘR⁸; a halogen atom; or OR⁸.

12. The compound according to claim 1 or N-oxide thereof, wherein R¹ represents a C1-C6 alkyl group having one or more substituents selected from a group consisting of halogen atom and cyano group; a cyclopropyl group optionally having one or more substituents selected from the group consisting of cyano group and halogen atom; S(O)ₘR⁸; or OR⁸.

13. The compound according to claim 1 or N-oxide thereof, wherein R² represents an ethyl group.

14. The compound according to claim 1 or N-oxide thereof, wherein Z represents an oxygen atom.

15. A composition for controlling harmful arthropod which comprises the compound of claim 1 or N-oxide thereof.

16. A composition, comprising:
one or more ingredients selected from the group consisting of the following Group (a), Group (b), Group (c), and Group (d); and
the compound of claim 1 or N-oxide thereof:
Group (a): a group consisting of insecticidal ingredients, miticidal ingredients, and nematicidal ingredients;
Group (b): fungicidal ingredients,
Group (c): plant growth modulating ingredients; and
Group (d): repellent ingredients.

17. (canceled)

18. A seed or vegetative reproductive organ carrying an effective amount of the compound according to claim 1 or N-oxide thereof.

19-20. (canceled)

21. A compound represented by formula (III): 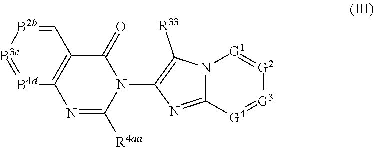
wherein
R³³ represents a hydrogen atom or halogen atom,
a combination of B^{2b}, B^{3c} and B^{4d} represents
a combination in which B^{2b} represents CR¹, B^{3c} represents a nitrogen atom or CR^{6cc}, and B^{4d} represents a nitrogen atom or CR^{6dd};
a combination in which B^{2b} represents a nitrogen atom or CR^{6bb}, B^{3c} represents CR¹, and B^{4d} represents a nitrogen atom or CR^{6dd}; or
a combination in which B^{2b} represents a nitrogen atom or CR^{6bb}, B^{3c} represents a nitrogen atom or CR^{6cc}, and B^{4d} represents CR¹,
R¹ represents a C1-C6 chain hydrocarbon group having one or more substituents selected from the group consisting of cyano group and halogen atom, a C3-C4 cycloalkyl group optionally having one or more substituents selected from the group consisting of cyano group and halogen atom, S(O)ₘR⁸, OR⁸, halogen atom or OS(O)₂R⁸,
m represents 0, 1 or 2,
R⁸ represents a C1-C6 chain hydrocarbon group having one or more substituents selected from the group consisting of cyano group and halogen atom; or a C3-C4 cycloalkyl group optionally having one or more substituents selected from the group consisting of cyano group and halogen atom,
R^{4aa}, R^{6bb}, R^{6cc} and R^{6dd} are identical to or different from each other and each represents a C1-C6 chain hydrocarbon group optionally having one or more halogen atoms, halogen atom or a hydrogen atom,
G¹ represents a nitrogen atom or CR^{3a},
G² represents CR^{3b},
G³ represents a nitrogen atom or CR^{3c},
G⁴ represents a nitrogen atom or CR^{3d},
R^{3a}, R^{3c} and R^{3d} are identical to or different from each other and each represents a C1-C6 chain hydrocarbon group optionally having one or more substituents selected from Group B, a C3-C7 cycloalkyl group optionally having one or more substituents selected from Group E, a phenyl group optionally having one or more substituents selected from Group H, 5 or 6 membered aromatic heterocyclic group optionally having one or more substituents selected from Group H, OR¹², NR¹¹R¹², NR^{11a}R^{12a}, NR²⁴NR¹¹R¹², NR²⁴OR¹¹, NR¹¹C(O)R¹³, NR²⁴NR¹¹C(O)R¹³, NR¹¹C(O)OR¹⁴, NR²⁴NR¹¹C(O)OR¹⁴, NR¹¹C(O)NR³¹R³², NR²⁴NR¹¹C(O)NR³¹R³², N═CHNR³¹R³², N═S(O)ₚR¹⁵R¹⁶, C(O)R¹³, C(O)R¹⁷, C(O)NR³¹R³², C(O)NR¹¹S(O)₂R²³, CR^{3c}═NOR¹⁷, NR¹¹CR²⁴═NOR¹⁷, S(O)_{q}R²³, a cyano group, a nitro group, a hydrogen atom, or a halogen atom,
R^{3b} represents a C1-C6 chain hydrocarbon group optionally having one or more substituents selected from Group B, a C3-C7 cycloalkyl group optionally having one or more substituents selected from Group E, a phenyl group optionally having one or more substituents selected from Group H, a 5 or 6 membered aromatic heterocyclic group optionally having one or more substituents selected from Group H, OR¹², NR¹¹R¹², NR^{11a}R^{12a}, NR²⁴NR¹¹R¹², NR²⁴OR¹¹, NR¹¹C(O)R¹³, NR²⁴NR¹¹C(O)R¹³, NR¹¹C(O)OR¹⁴, NR²⁴NR¹¹C(O)OR¹⁴, NR¹¹C(O)NR³¹R³², NR²⁴NR¹¹C(O)NR³¹R³², N═CHNR³¹R³², N═S(O)ₚR¹⁵R¹⁶, C(O)R¹³, C(O)OR¹⁷, C(O)NR³¹R³², C(O)NR¹¹S(O)₂R²³, CR^{3c}═NOR¹⁷, NR¹¹CR²⁴═NOR¹⁷, S(O)_{q}R²³, a cyano group, a nitro group, or a hydrogen atom,
p represents 0 or 1,
q represents 0 or 1,
R^{3c} represents a C1-C6 chain hydrocarbon group optionally having one or more halogen atoms, a halogen atom OR³⁵, NR³⁶R³⁷, or a hydrogen atom,
R³⁵ represents a C1-C6 chain hydrocarbon group optionally having one or more halogen atoms,
R¹⁷ represents a C1-C6 chain hydrocarbon group optionally having one or more halogen atoms, a phenyl group optionally having one or more substituents selected from Group D, or a hydrogen atom,
R¹¹, R²⁴, R³⁶ and R³⁷ are identical to or different from each other and each represents a C1-C6 chain hydrocarbon group optionally having one or more halogen atoms, or a hydrogen atom,
R¹² represents a C1-C6 chain hydrocarbon group optionally having one or more substituents selected from Group F, a C3-C7 cycloalkyl group optionally having one or more substituents selected from Group J, a C3-C7 cycloalkenyl group optionally having one or more substituents selected from Group J, a phenyl group optionally having one or more substituents selected from Group D, a 6 membered aromatic heterocyclic group optionally having one or more substituents selected from Group D, a hydrogen atom, or S(O)₂R²³,
R²³ represents a C1-C6 chain hydrocarbon group optionally having one or more halogen atoms, or a phenyl group optionally having one or more substituents selected from Group D,
R^{11a} and R^{12a} combined together with a nitrogen atom to which they are attached represent a 3 to 7 membered nonaromatic heterocyclic group optionally having one or more substituents selected from Group E,
R¹³ represents a C1-C6 chain hydrocarbon group optionally having one or more halogen atoms, a C3-C7 cycloalkyl group optionally having one or more halogen atoms, a (C3-C6 cycloalkyl)C1-C3 alkyl group optionally having one or more halogen atoms, a phenyl group optionally having one or more substituents selected from Group D, a 5 or 6 membered aromatic heterocyclic group optionally having one or more substituents selected from Group D, or a hydrogen atom,
R¹⁴ represents a C1-C6 chain hydrocarbon group optionally having one or more halogen atoms, a C3-C7 cycloalkyl group optionally having one or more halogen atoms, a (C3-C6 cycloalkyl)C1-C3 alkyl group optionally having one or more halogen atoms, or a phenyl C1-C3 alkyl group {the phenyl moiety in the phenyl C1-C3 alkyl group may have optionally one or more substituents selected from Group D},
R¹⁵ and R¹⁶ are identical to or different from each other and each represents a C1-C6 alkyl group optionally having one or more halogen atoms,
R³¹ represents a C1-C6 alkyl group optionally having one or more halogen atoms, or a hydrogen atom,
R³² represents a C1-C6 chain hydrocarbon group optionally having one or more substituents selected from Group F, a C3-C7 cycloalkyl group optionally having one or more substituents selected from Group J, or a hydrogen atom,
Group B: a group consisting of a C1-C6 alkoxy group optionally having one or more halogen atoms, a C3-C6 alkenyloxy group optionally having one or more halogen atoms, a C3-C6 alkynyloxy group optionally having one or more halogen atoms, a C1-C6 alkylsulfanyl group optionally having one or more halogen atoms, a C1-C6 alkylsulfinyl group optionally having one or more halogen atoms, a C1-C6 alkylsulfonyl group optionally having one or more halogen atoms, a C3-C6 cycloalkyl group optionally having one or more halogen atoms, a C1-C6 alkylamino group optionally having one or more halogen atoms, a di(C1-C4 alkyl)amino group optionally having one or more halogen atoms, a C2-C6 alkylcarbonyl group optionally having one or more halogen atoms, a C2-C6 alkoxycarbonyl group optionally having one or more halogen atoms, a C2-C6 alkoxycarbonyloxy group optionally having one or more halogen atoms, an aminocarbonyl group, a C1-C6 alkylaminocarbonyl group optionally having one or more halogen atoms, a di(C1-C4 alkyl)aminocarbonyl group optionally having one or more halogen atoms, a C2-C6 alkoxycarbonylamino group optionally having one or more halogen atoms, a (C2-C6 alkoxycarbonyl)(C1-C6 alkyl) amino group optionally having one or more halogen atoms, a cyano group, an amino group, a nitro group, a hydroxy group, and a halogen atom,
Group D: a group consisting of a C1-C6 chain hydrocarbon group optionally having one or more halogen atoms, a C1-C6 alkoxy group optionally having one or more halogen atoms, a C3-C6 alkenyloxy group optionally having one or more halogen atoms, a C3-C6 alkynyloxy group optionally having one or more halogen atoms, a C1-C6 alkylsulfanyl group optionally having one or more halogen atoms, a C1-C6 alkylsulfinyl group optionally having one or more halogen atoms, a C1-C6 alkylsulfonyl group optionally having one or more halogen atoms, a C3-C6 cycloalkyl group optionally having one or more halogen atoms, a C1-C6 alkylamino group optionally having one or more halogen atoms, a di(C1-C4 alkyl)amino group optionally having one or more halogen atoms, a C2-C6 alkylcarbonyl group optionally having one or more halogen atoms, a C2-C6 alkoxycarbonyl group optionally having one or more halogen atoms, a C2-C6 alkoxycarbonyloxy group optionally having one or more halogen atoms, an aminocarbonyl group, a C1-C6 alkylaminocarbonyl group optionally having one or more halogen atoms, a di(C1-C4 alkyl)aminocarbonyl group optionally having one or more halogen atoms, a C2-C6 alkoxycarbonylamino group optionally having one or more halogen atoms, a (C2-C6 alkoxycarbonyl)(C1-C6 alkyl) amino group optionally having one or more halogen atoms, a cyano group, an amino group, a nitro group, a hydroxy group, and a halogen atom,
Group E: a group consisting of a C1-C6 chain hydrocarbon group optionally having one or more halogen atoms, a C1-C6 alkoxy group optionally having one or more halogen atoms, a C3-C6 alkenyloxy group optionally having one or more halogen atoms, a C3-C6 alkynyloxy group optionally having one or more halogen atoms, a C1-C6 alkylamino group optionally having one or more halogen atoms, a di(C1-C4 alkyl)amino group optionally having one or more halogen atoms, a C2-C6 alkylcarbonyl group optionally having one or more halogen atoms, a C2-C6 alkoxycarbonyl group optionally having one or more halogen atoms, a C2-C6 alkoxycarbonyloxy group optionally having one or more halogen atoms, an aminocarbonyl group, a C1-C6 alkylaminocarbonyl group optionally having one or more halogen atoms, a di(C1-C4 alkyl)aminocarbonyl group optionally having one or more halogen atoms, a C2-C6 alkoxycarbonylamino group optionally having one or more halogen atoms, a (C2-C6 alkoxycarbonyl)(C1-C6 alkyl) amino group optionally having one or more halogen atoms, a cyano group, an amino group, a nitro group, a hydroxy group, an oxo group, and a halogen atom,
Group F: a group consisting of a C3-C7 cycloalkyl group optionally having one or more halogen atoms, a phenyl group optionally having one or more substituents selected from Group D, a 5 or 6 membered aromatic heterocyclic group optionally having one or more substituents selected from Group D, a C1-C6 alkoxy group optionally having one or more halogen atoms, a C1-C6 alkylamino group optionally having one or more halogen atoms, a di(C1-C4 alkyl)amino group optionally having one or more halogen atoms, a cyano group, an amino group, a nitro group, a hydroxy group, and a halogen atom,
Group H: a group consisting of a C1-C6 chain hydrocarbon group optionally having one or more halogen atoms, a C3-C6 cycloalkyl group optionally having one or more halogen atoms, a phenyl group optionally having one or more substituents selected from Group D, a 5 or 6 membered aromatic heterocyclic group optionally having one or more substituents selected from Group D, a C1-C6 alkoxy group optionally having one or more halogen atoms, a C1-C6 alkylamino group optionally having one or more halogen atoms, a di(C1-C4 alkyl)amino group optionally having one or more halogen atoms, a C2-C6 alkylcarbonyl group optionally having one or more halogen atoms, a C2-C6 alkoxycarbonyl group optionally having one or more halogen atoms, a C2-C6 alkoxycarbonyloxy group optionally having one or more halogen atoms, an aminocarbonyl group, a (C1-C6 alkyl)aminocarbonyl group optionally having one or more halogen atoms, a di(C1-C4 alkyl)aminocarbonyl group optionally having one or more halogen atoms, a C2-C6 alkoxycarbonylamino group optionally having one or more halogen atoms, a (C2-C6 alkoxycarbonyl)(C1-C6 alkyl) amino group optionally having one or more halogen atoms, a cyano group, an amino group, a nitro group, a hydroxy group, and a halogen atom.

22. (canceled)
